SEQUENCE LISTING (1 sequence)

SEQ ID NO: 1 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

(19) United States
(12) Patent Application Publication
(10) Pub. No.: US 2025/0270191 A1
(43) Pub. Date: Aug. 28, 2025

(54) QUINOLONE BCL6 BIFUNCTIONAL DEGRADERS

(71) Applicant: Treeline Biosciences, Inc., STAMFORD, CT (US)

(72) Inventors: Kevin M. Oberg, San Diego, CA (US); Joshua D. Hansen, San Diego, CA (US); Jeffrey A. Engelman, Brookline, MA (US); Joel D. Leverson, San Diego, CA (US); Tami J. Marrone, Carlsbad, CA (US); Matthew H. McNeill, San Clemente, CA (US); Mark A. Nagy, Encinitas, CA (US); Kristin D. Schleicher, San Diego, CA (US); Ming Yan, San Diego, CA (US)

(21) Appl. No.: 18/874,511

(22) PCT Filed: Jun. 5, 2023
(86) PCT No.: PCT/US2023/067919

§ 371 Date: Dec. 12, 2024

Related U.S. Application Data
(60) Provisional application No. 63/501,082, filed on May 9, 2023.
(60) Provisional application No. 63/497,063, filed on Apr. 19, 2023.
(60) Provisional application No. 63/444,801, filed on Feb. 10, 2023.
(60) Provisional application No. 63/444,778, filed on Feb. 10, 2023.
(60) Provisional application No. 63/420,421, filed on Oct. 28, 2022.
(60) Provisional application No. 63/420,411, filed on Oct. 28, 2022.
(60) Provisional application No. 63/395,630, filed on Aug. 5, 2022.
(60) Provisional application No. 63/395,638, filed on Aug. 5, 2022.
(60) Provisional application No. 63/351,697, filed on Jun. 13, 2022.
(60) Provisional application No. 63/351,715, filed on Jun. 13, 2022.

(51) Int. Cl. C07D 401/14; A61K 31/496; A61K 31/506; A61K 31/5377; A61K 45/06; A61K 47/55; C07D 413/14; C07D 471/04; C07D 471/10; C07D 487/08

(52) U.S. Cl. CPC C07D 401/14; A61K 31/496; A61K 31/506; A61K 31/5377; A61K 45/06; A61K 47/55; C07D 413/14; C07D 471/04; C07D 471/10; C07D 487/08

(57) ABSTRACT

This disclosure provides compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, that induce degradation of a BCL6 protein. These compounds are useful, for example, for treating a cancer in a subject (e.g., a human). This disclosure also provides compositions containing the same as well as methods of using and making the same.

### CROSS-REFERENCE TO RELATED APPLICATIONS

This application claims priority to U.S. Provisional Application Ser. No. 63/501,082, filed May 9, 2023; 63/497,063, filed Apr. 19, 2023; 63/444,778, filed Feb. 10, 2023; 63/444,801, filed Feb. 10, 2023; 63/420,421, filed Oct. 28, 2022; 63/420,411, filed Oct. 28, 2022; 63/395,638, filed Aug. 5, 2022; 63/395,630, filed Aug. 5, 2022; 63/351,715, filed Jun. 13, 2022; 63/351,697, filed Jun. 13, 2022; each of which is incorporated by reference in its entirety herein.

### DESCRIPTION OF THE TEXT FILE SUBMITTED ELECTRONICALLY

This application contains a Sequence Listing which has been submitted electronically in XML format. The Sequence Listing XML is incorporated herein by reference. Said XML file, created on May 26, 2023, is named TLS-043WO_SL.xml and is 2,663 bytes in size.

### TECHNICAL FIELD

This disclosure provides compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, that induce degradation of a BCL6 protein. These compounds are useful, for example, for treating cancer in a subject (e.g., a human). This disclosure also provides compositions containing the same as well as methods of using and making the same.

### BACKGROUND

B-cell lymphoma 6 (BCL6) protein is a transcriptional repressor involved in the formation and maintenance of germinal centers (GCs) within lymphoid follicles. It controls the functions of the GC and coordinates the activities of signaling mediators in the maturation of GC B cells. There are over 1000 known or putative BCL6 target genes, including MYC, BCL2, genes related to DNA damage response (e.g., ATR, TP53), and cell cycle checkpoint control (e.g., CDKN1A, CDKN1B). BCL6 is expressed in the dark zone cells of GCs, where somatic hypermutation is allowed to occur to generate high-affinity B-cell receptors. Overexpression or loss of control of BCL6, for example by translocation, can permit maintenance of the pro-hypermutation functions and abrogation of the antitumor functions of BCL6.

### SUMMARY

Provided herein are compounds of Formula (I):

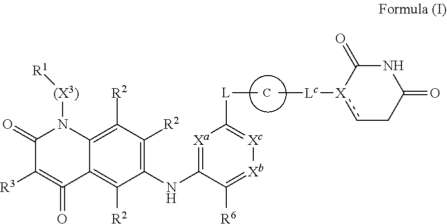

or pharmaceutically acceptable salts thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
R³ is selected from the group consisting of:
-A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂(C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1}, L^{A3}, and L^{A4}, provided that 1-3 occurrences of L^{A} is L^{A4};

### (BB)

n1 is an integer from 0 to 20; and
each L^{A} is independently selected from the group consisting of: L^{A1} and L^{A3},
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{c};
Ring C is selected from the group consisting of:

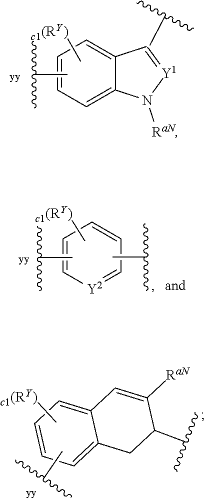

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂(C₁₋₆ alkyl);
(m) S(O)₀₋₂(C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R⁸;
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h},
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

Also provided herein are pharmaceutical compositions comprising a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))), or a pharmaceutically acceptable salt thereof, and a pharmaceutically acceptable carrier.

Provided herein are methods for treating cancer in a subject in need thereof, the methods comprising administering to the subject a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))), or a pharmaceutically acceptable salt thereof, or a pharmaceutical composition as provided herein.

Also provided herein are BCL6 proteins non-covalently bound with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))), or a pharmaceutically acceptable salt thereof.

Also provided herein are ternary complexes comprising a BCL6 protein, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))), or a pharmaceutically acceptable salt thereof, and a CRBN protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In the case of conflict between the present disclosure and any content incorporated by reference, the present disclosure controls.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, that induce degradation of a BCL6 protein. These compounds are useful, e.g., for treating a cancer. This disclosure also provides compositions containing the compounds provided herein as well as methods of using and making the same.

Upon antigen challenge, germinal centers (GCs) are formed in lymphoid follicles, and B-cells in the dark zone of GCs undergo rapid proliferation and somatic hypermutation, both of their immunoglobin variable genes to generate high-affinity B-cell receptors, as well as of other genes including BCL6. BCL6 is often considered to be a ‘master regulator’ of the GC reaction. In some cancers, BCL6 can be mutated, translocated, and/or BCL6 expression can be upregulated. See, e.g., Leeman-Neill and Bhagat, Expert Opinion on Therapeutic Targets 22.2 (2018): 143-152; Mlynarczyk and Melnick. Immunological Reviews 288.1 (2019): 214-239.

The BCL6 protein has multiple domains, including a BTB domain, an RD2 domain, and a DNA binding domain. The N-terminal BTB domain is the site of homodimerization of BCL6, and the interface of the monomers forms the “lateral groove”, which is a binding site for endogenous co-repressors of BCL6, such as SMRT, NCOR, and BCOR. See, e.g., Cardenas, Mariano G., et al. Clinical Cancer Research 23.4 (2017): 885-893.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the cereblon (CRBN) E3 ligase (also referred to herein as a CRBN protein) is used.

A degradation approach for a target protein can have potential advantages compared to, e.g., small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2021/077010 and WO 2022/221673; McCoull, William, et al., ACS Chemical Biology 13.11 (2018): 3131-3141; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12.

### Compound Embodiments

Provided herein are compounds of Formula (I):

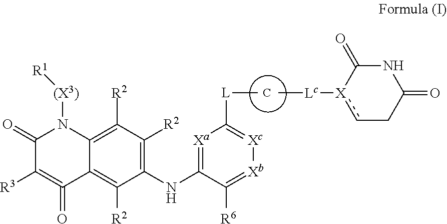

or pharmaceutically acceptable salts thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
R³ is selected from the group consisting of:
-A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂(C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1}, L^{A3}, and L^{A4}, provided that 1-3 occurrences of L^{A} is L^{A4}.

### (BB)

n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{c};
Ring C is selected from the group consisting of:

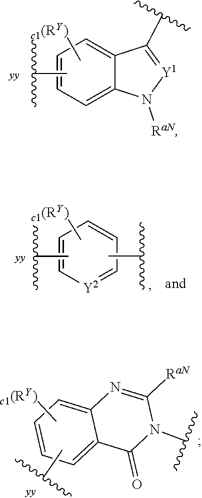

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R¹)₂;
(l) S(O)₀₋₂(C₁₋₆ alkyl);
(m) S(O)₀₋₂(C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h},
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

In some embodiments, it is provided that when X is N, then Ring C is

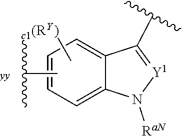

In some embodiments, R³ is -A¹-C(R⁴R⁴)-A².

In some embodiments, A¹ is —O—.

In some embodiments, each R⁴ is H.

In some embodiments, A² is —C(O)NH₂ or —C(O)NR^{3A}R^{f}. In some embodiments, R^{3A} is C₁₋₃ alkyl optionally substituted with 1-6 R^{c}. For example, A² can be —C(O)NH₂, —C(O)NHMe, or —C(O)NMe₂. For example, A² can be —C(O)NHMe.

In some embodiments, R³ is

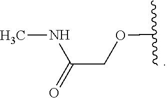 [SMILES: *OCC(=O)NC]

In some embodiments, X^{a} is N; X^{c} is N; and X^{b} is CR^{x1} (e.g., CH).

In some embodiments, X^{a} is CH; X_{c} is N; and X^{b} is CR^{x1} (e.g., CH).

In some embodiments, R⁶ is —Cl or —F.

In some embodiments, each R² is H.

In some embodiments, one R² is selected from the group consisting of: halo, cyano, C₁₋₃ alkyl, C₁₋₃ haloalkyl, C₁₋₃ alkoxy, and C₁₋₃ haloalkoxy; and each remaining R² is H.

In some embodiments, m3 is 0.

In some embodiments, m3 is 1; and X³ is C₁₋₃ alkylene.

In some embodiments, R¹ is H.

In some embodiments, m3 is 1; X³ is methylene, ethylene, or isopropylene; and R¹ is H.

In some embodiments, R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{e}; each R² is H; X^{a} is N or CH; X^{c} is N; X^{b} is CH; and R⁶ is —F or —Cl. In some embodiments, m3 is 1; X³ is methylene, ethylene, or isopropylene; and R¹ is H. In some embodiments, X^{a} is N. In some embodiments, A² is C(O)NHMe.

In some embodiments, Ring C is

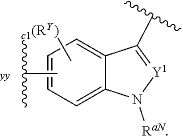

In some embodiments, Ring C is

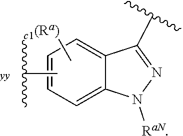

In some embodiments, Ring C is

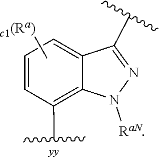

In some embodiments, c1 is 0. In some embodiments, c1 is 1; and R^{Y} is halo (e.g., —F). In some embodiments, R^{aN} is C₁₋₃ alkyl (e.g., methyl).

In some embodiments, Ring C is

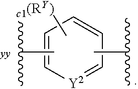

In some embodiments, c1 is 0. In some embodiments, c1 is 1; and R^{Y} is halo (e.g., —F). In some embodiments, R^{aN} is C₁₋₃ alkyl (e.g., methyl).

In some embodiments, c1 is 0. In some embodiments, c1 is 1; and R^{Y} is halo (e.g., —F).

In some embodiments, X is CH.

In some embodiments, L^{C} is a bond.

In some embodiments, the

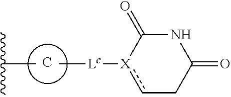

moiety is selected from the group consisting of:

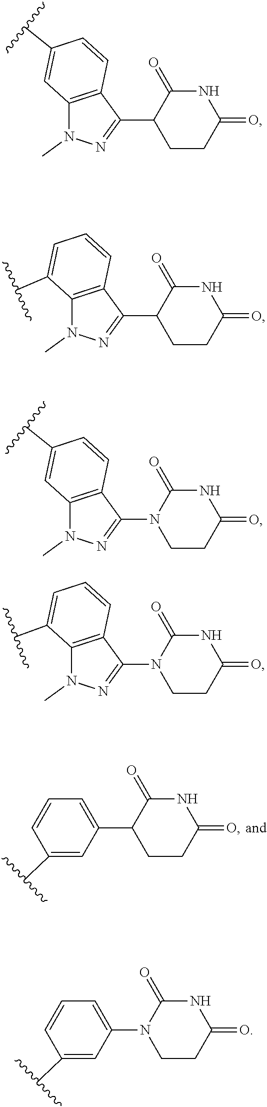

In some embodiments, the

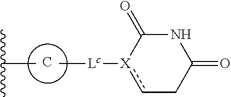

moiety is selected from the group consisting of:

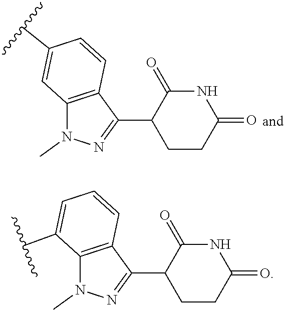

In some embodiments, the

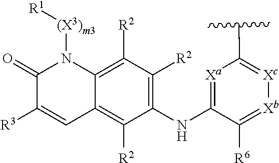

moiety is

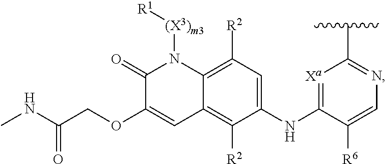

wherein: m3 is 1; X³ is C₁₋₃ alkylene; R¹ is H; X^{a} is CH or N; X^{c} is N; R⁶ is —F or —Cl; and the

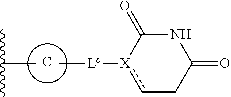

moiety is selected from the group consisting of:

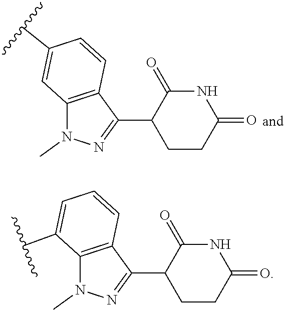

In some embodiments, —(X³)ₘ₃—R¹ is methyl, ethyl, or isopropyl. In some embodiments, X^{a} is CH. In some embodiments, X^{a} is N.

In some embodiments, L is -(L^{A})ₙ₁-, wherein L^{A} and n1 are defined according to (AA).

In some embodiments, n1 is an integer from 1 to 5.

In some embodiments, n1 is an integer from 2 to 4 (e.g., 2 or 3).

In some embodiments, L is selected from the group consisting of:
-L^{A4}-L^{A1}-L^{A4}-bb;
-L^{A4}-L^{A4}-bb;
L^{A4}-L^{A3}-L^{A4}-bb; and
-L^{A4}-L^{A1}-L^{A4}-L^{A3}-bb,
wherein bb represents the point of attachment to Ring C.

In some embodiments, each L^{A4} is independently a C₃₋₁₀ cycloalkylene or 4-12 membered heterocyclylene, each of which is optionally substituted with 1-6 R^{a}. In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, each L^{A4} is independently a 4-12 (e.g., 4-10) membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, each L^{A4} is independently a monocyclic 4-6 membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, one L^{A4} is a monocyclic 4-6 membered nitrogen-containing heterocyclylene optionally substituted with 1-3 R^{a}; and the other L^{A4} is a bicyclic 6-12 (e.g., 6-10) membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, one L^{A4} is a monocyclic 4-6 membered nitrogen-containing heterocyclylene optionally substituted with 1-3 R^{a}; and the other L^{A4} is a bicyclic spirocyclic 6-12 (e.g., 6-10) membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, one L^{A4} is a monocyclic 4-6 membered nitrogen-containing heterocyclylene optionally substituted with 1-3 R^{a}; and the other L^{A4} is a bicyclic bridged 6-12 (e.g., 6-10) membered nitrogen-containing heterocyclylene optionally substituted with 1-3 R^{a}. In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, each L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, L is -L^{A4}-L^{A1}-L^{A4}-_{bb}. In some embodiments, L^{A1} is —CH₂—, —CHMe-, or —CMe₂-.

In some embodiments, L is -L^{A4}-L^{A3}-L^{A4}-_{bb}. In some embodiments, L^{A3} is —C(═O) or —O—.

In some embodiments, L is -L^{A4}-L^{A1}-L^{A4}-L^{A3}-_{bb}, and L^{A3} is C(═O).

In some embodiments, L is selected from the group consisting of:
-L^{A4}-L^{A3}-_{bb};
-L^{A4}-L^{A1}-_{bb}; and
-L^{A4}-L^{A1}-L^{A3}-_{bb},
wherein bb represents the point of attachment to Ring C.

In some embodiments, L is -L^{A4}-L^{A3}-_{bb}. In some embodiments, L^{A3} is —NH— or —N(C₁₋₃ alkyl)- (e.g., —NH—). In some embodiments, L^{A4} is a 4-12 membered heterocyclylene optionally substituted with 1-6 R^{a}. In some embodiments, L^{A4} is a 4-12 (e.g., 6-10) membered nitrogen-containing heterocyclylene optionally substituted with 1-3 R^{a}. In some embodiments, L^{A4} is a bicyclic spirocyclic 6-12 (e.g., 6-10) membered nitrogen-containing heterocyclylene optionally substituted with 1-3 R^{a}. In some embodiments, L^{A4} contains 1-2 ring nitrogen atoms and no additional ring heteroatoms. In some embodiments, each R^{a} present on L^{A4} is independently selected from the group consisting of: —F, CN, C₁₋₃ alkoxy, OH, and C₁₋₃ alkyl optionally substituted with 1-3 F.

In some embodiments, L is selected from the group consisting of the moieties delineated in Table L:

TABLE L
Examples when L is —L^{A4}-L^{A1}-L^{A4}-_{bb}

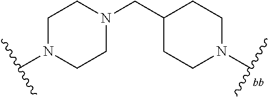 [SMILES: *N1CCC(CN2CCN(*)CC2)CC1]

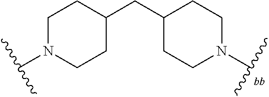 [SMILES: *N1CCC(CC2CCN(*)CC2)CC1]

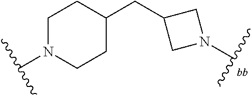 [SMILES: *N1CCC(CC2CN(*)C2)CC1]

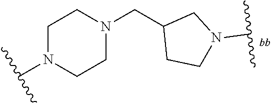 [SMILES: *N1CCN(CC2CCN(*)C2)CC1]

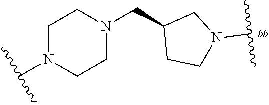 [SMILES: *N1CCN(C[C@@H]2CCN(*)C2)CC1]

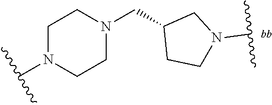 [SMILES: *N1CCN(C[C@H]2CCN(*)C2)CC1]

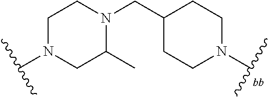 [SMILES: *N1CCC(CN2CCN(*)CC2C)CC1]

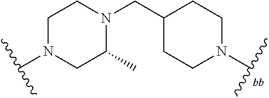 [SMILES: *N1CCC(CN2CCN(*)C[C@H]2C)CC1]

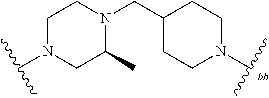 [SMILES: *N1CCC(CN2CCN(*)C[C@@H]2C)CC1]

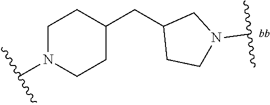 [SMILES: *N1CCC(CC2CCN(*)C2)CC1]

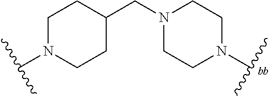 [SMILES: *N1CCC(CN2CCN(*)CC2)CC1]

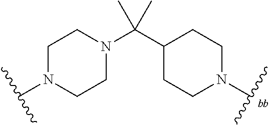 [SMILES: *N1CCC(C(C)(C)N2CCN(*)CC2)CC1]

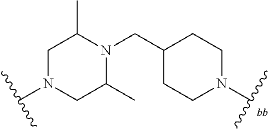 [SMILES: *N1CCC(CN2C(C)CN(*)CC2C)CC1]

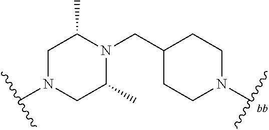 [SMILES: *N1CCC(CN2[C@H](C)CN(*)C[C@@H]2C)CC1]

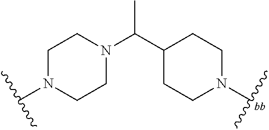 [SMILES: *N1CCC(C(C)N2CCN(*)CC2)CC1]

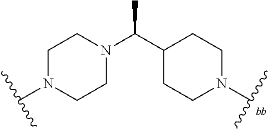 [SMILES: *N1CCC([C@H](C)N2CCN(*)CC2)CC1]

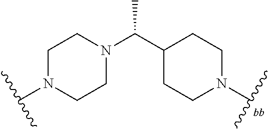 [SMILES: *N1CCC([C@@H](C)N2CCN(*)CC2)CC1]

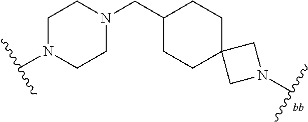 [SMILES: *N1CCN(CC2CCC3(CC2)CN(*)C3)CC1]

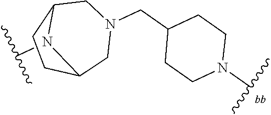 [SMILES: *N1CCC(CN2CC3CCC(C2)N3*)CC1]

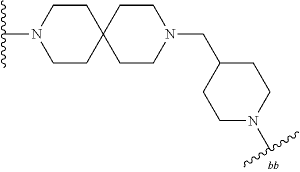 [SMILES: *N1CCC(CN2CCC3(CCN(*)CC3)CC2)CC1]

Examples when —L is L^{A4}-L^{A4}-_{bb}

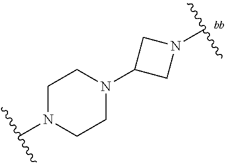

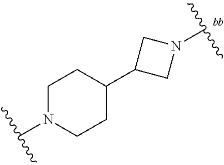

Examples when L is —L^{A4}-L^{A3}-L^{A4}-_{bb}

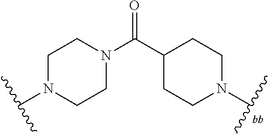 [SMILES: *N1CCC(C(=O)N2CCN(*)CC2)CC1]

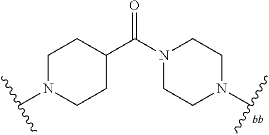 [SMILES: *N1CCC(C(=O)N2CCN(*)CC2)CC1]

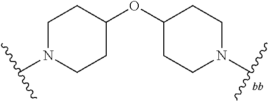 [SMILES: *N1CCC(OC2CCN(*)CC2)CC1]

Examples when L is —L^{A4}-L^{A1}-L^{A4}-L^{A3}-_{bb}

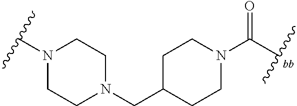 [SMILES: *C(=O)N1CCC(CN2CCN(*)CC2)CC1]

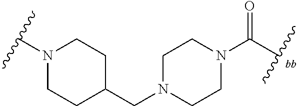 [SMILES: *C(=O)N1CCN(CC2CCN(*)CC2)CC1]

Examples when L is —L^{A4}-L^{A3}-_{bb}

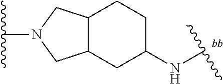 [SMILES: *NC1CCC2CN(*)CC2C1]

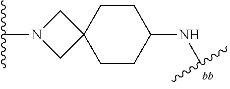 [SMILES: *NC1CCC2(CC1)CN(*)C2]

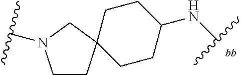 [SMILES: *NC1CCC2(CC1)CCN(*)C2]

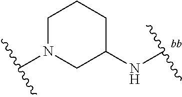 [SMILES: *NC1CCCN(*)C1]

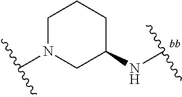 [SMILES: *N[C@@H]1CCCN(*)C1]

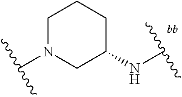 [SMILES: *N[C@H]1CCCN(*)C1]

Examples when L is —L^{A4}-L^{A1}-L^{A3}-_{bb}

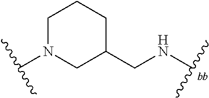 [SMILES: *NCC1CCCN(*)C1]

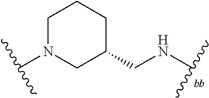 [SMILES: *NC[C@H]1CCCN(*)C1]

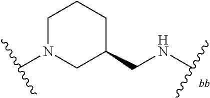 [SMILES: *NC[C@@H]1CCCN(*)C1]

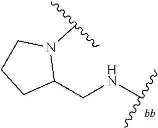 [SMILES: *NCC1CCCN1*]

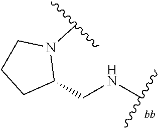 [SMILES: *NC[C@@H]1CCCN1*]

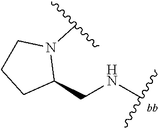 [SMILES: *NC[C@H]1CCCN1*]

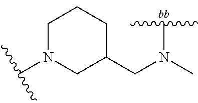 [SMILES: *N(C)CC1CCCN(*)C1]

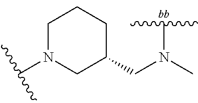 [SMILES: *N(C)C[C@H]1CCCN(*)C1]

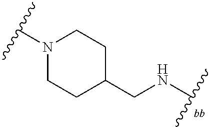 [SMILES: *NCC1CCN(*)CC1]

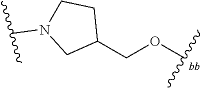 [SMILES: *OCC1CCN(*)C1]

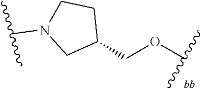 [SMILES: *OC[C@H]1CCN(*)C1]

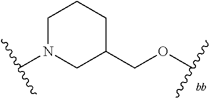 [SMILES: *OCC1CCCN(*)C1]

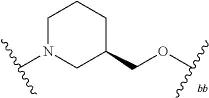 [SMILES: *OC[C@@H]1CCCN(*)C1]

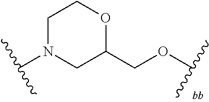 [SMILES: *OCC1CN(*)CCO1]

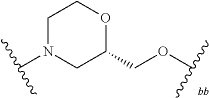 [SMILES: *OC[C@@H]1CN(*)CCO1]

Examples when L is —L^{A4}-(L^{A1})₂₋₄-L^{A3}-_{bb}

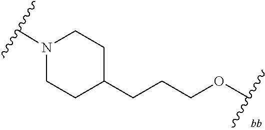 [SMILES: *OCCCC1CCN(*)CC1]

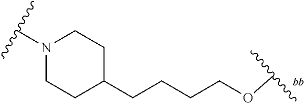 [SMILES: *OCCCCC1CCN(*)CC1]

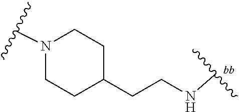 [SMILES: *NCCC1CCN(*)CC1]

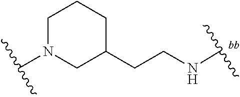 [SMILES: *NCCC1CCCN(*)C1]

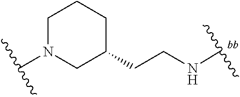 [SMILES: *NCC[C@H]1CCCN(*)C1]

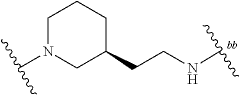 [SMILES: *NCC[C@@H]1CCCN(*)C1]

Examples when L is —L^{A4}-_{bb}

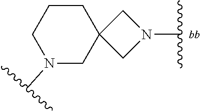 [SMILES: *N1CCCC2(C1)CN(*)C2]

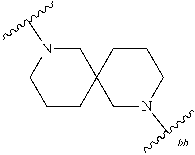 [SMILES: *N1CCCC2(CCCN(*)C2)C1]

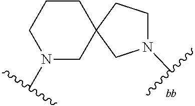 [SMILES: *N1CCCC2(CCN(*)C2)C1]

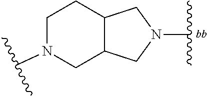 [SMILES: *N1CCC2CN(*)CC2C1]

Examples when L is —L^{A3}-L^{A1}-L^{A4}-_{bb}

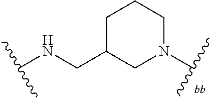 [SMILES: *NCC1CCCN(*)C1]

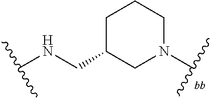 [SMILES: *NC[C@@H]1CCCN(*)C1]

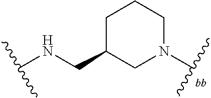 [SMILES: *NC[C@H]1CCCN(*)C1]

Examples when L is —L^{A3}-L^{A1}-L^{A4}-
(L^{A1})₀₋₁-L^{A3}-_{bb}

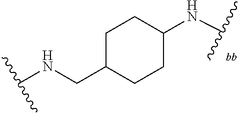 [SMILES: *NCC1CCC(N*)CC1]

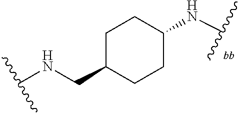 [SMILES: *NC[C@H]1CC[C@H](N*)CC1]

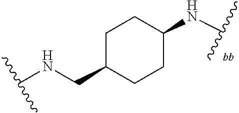 [SMILES: *NC[C@H]1CC[C@@H](N*)CC1]

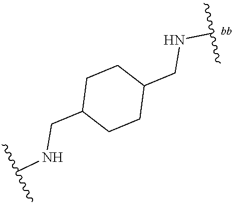 [SMILES: *NCC1CCC(CN*)CC1]

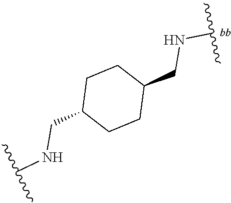 [SMILES: *NC[C@H]1CC[C@H](CN*)CC1]

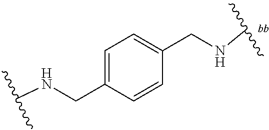 [SMILES: *NCc1ccc(CN*)cc1]

Example when L is —L^{A4}-L^{A1}-L^{A3}-L^{A3}-_{bb}

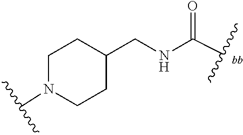

wherein bb represents the point of attachment to Ring C.

In some embodiments, L is selected from the group consisting of the moieties delineated in Table L1-a:

TABLE L1-a

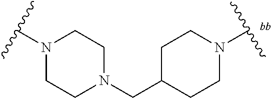

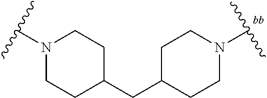

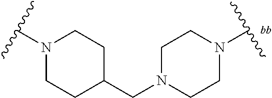

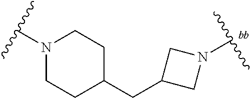

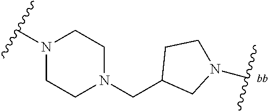

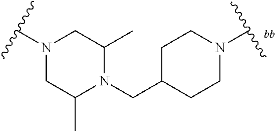

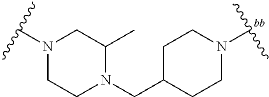

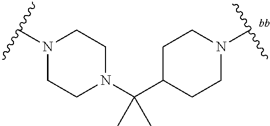

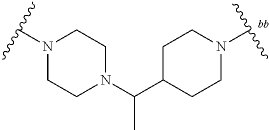

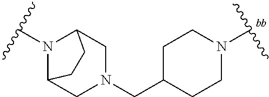

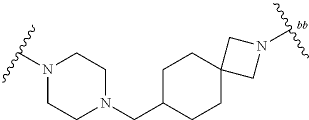

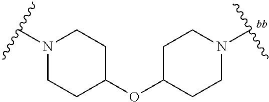

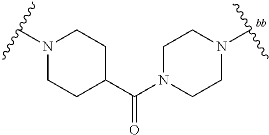

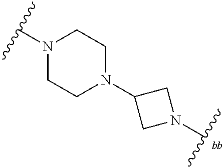

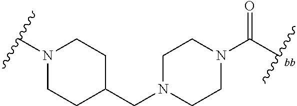

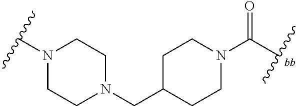

wherein bb represents the point of attachment to Ring C.

In some embodiments, L is selected from the group consisting of the moieties delineated in Table L2-a:

TABLE L2-a

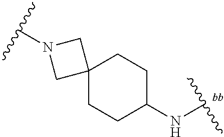

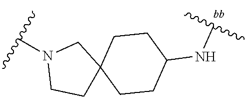

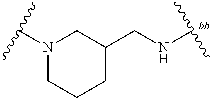

wherein bb represents the point of attachment to Ring C.

In some embodiments, the compounds of Formula (I) are compounds of Formula (I-aa):

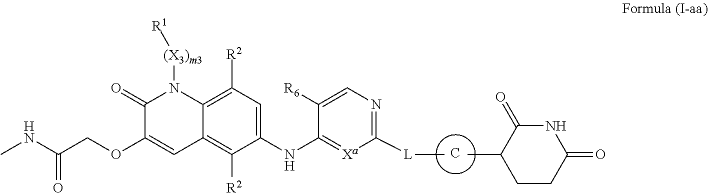

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

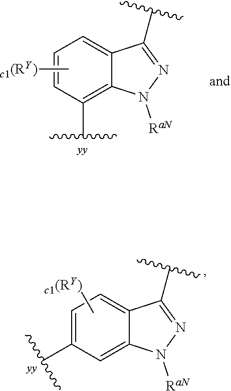

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:
-L^{A4}-L^{A1}-L^{A4}-_{bb};
-L^{A4}-O-L^{A4}-_{bb};
-L^{A4}-C(═O)-L^{A4}-_{bb}; and
-L^{A4}-L^{A1}-L^{A4}-C(═O)-_{bb},
wherein bb represents the point of attachment to Ring C;
L^{A1} is CH₂, CHMe, or CMe₂; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa), each L^{A4} is independently a monocyclic 4-6 membered nitrogen-containing heterocyclylene optionally substituted with 1-3 R^{a},
wherein each L^{A4} contains 1-2 ring nitrogen atoms and no additional ring heteroatoms.

In some embodiments of Formula (I-aa), one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

In some embodiments of Formula (I-aa), one L^{A4} is a monocyclic 4-6 membered nitrogen-containing heterocyclylene optionally substituted with 1-3 R^{a}; and
the other L^{A4} is a bicyclic bridged 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

In some embodiments of Formula (I-aa), L is -L^{A4}-L^{A1}-L^{A4}-_{bb}; and L^{A1} is CH₂ or CHMe.

In some embodiments of Formula (I-aa), L is selected from the group consisting of the moieties delineated in Table L1-a.

In some embodiments of Formula (I-aa), L is selected from the group consisting of the moieties delineated in Table L-I-a, wherein bb represents the point of attachment to Ring C.

TABLE L-I-a

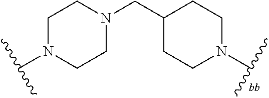 [SMILES: *N1CCC(CN2CCN(*)CC2)CC1]

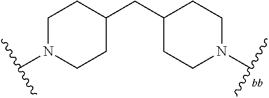 [SMILES: *N1CCC(CC2CCN(*)CC2)CC1]

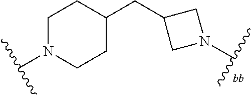 [SMILES: *N1CCC(CC2CN(*)C2)CC1]

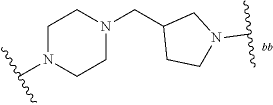 [SMILES: *N1CCN(CC2CCN(*)C2)CC1]

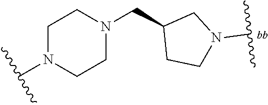 [SMILES: *N1CCN(C[C@@H]2CCN(*)C2)CC1]

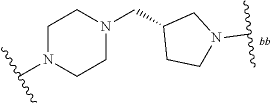 [SMILES: *N1CCN(C[C@H]2CCN(*)C2)CC1]

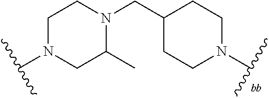 [SMILES: *N1CCC(CN2CCN(*)CC2C)CC1]

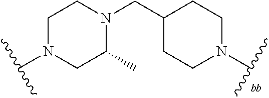 [SMILES: *N1CCC(CN2CCN(*)C[C@H]2C)CC1]

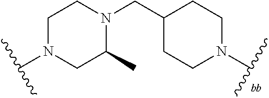 [SMILES: *N1CCC(CN2CCN(*)C[C@@H]2C)CC1]

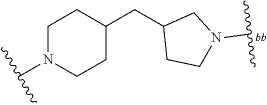 [SMILES: *N1CCC(CC2CCN(*)C2)CC1]

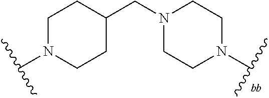 [SMILES: *N1CCC(CN2CCN(*)CC2)CC1]

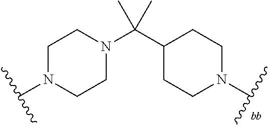 [SMILES: *N1CCC(C(C)(C)N2CCN(*)CC2)CC1]

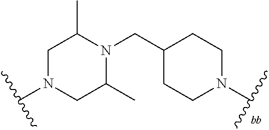 [SMILES: *N1CCC(CN2C(C)CN(*)CC2C)CC1]

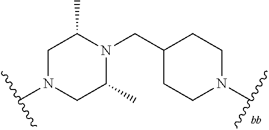 [SMILES: *N1CCC(CN2[C@H](C)CN(*)C[C@@H]2C)CC1]

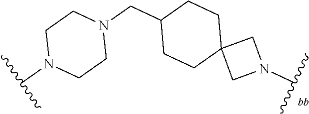 [SMILES: *N1CCN(CC2CCC3(CC2)CN(*)C3)CC1]

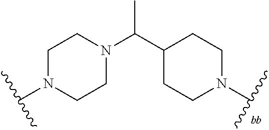 [SMILES: *N1CCC(C(C)N2CCN(*)CC2)CC1]

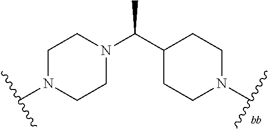 [SMILES: *N1CCC([C@H](C)N2CCN(*)CC2)CC1]

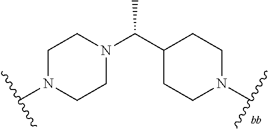 [SMILES: *N1CCC([C@@H](C)N2CCN(*)CC2)CC1]

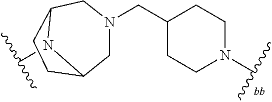 [SMILES: *N1CCC(CN2CC3CCC(C2)N3*)CC1]

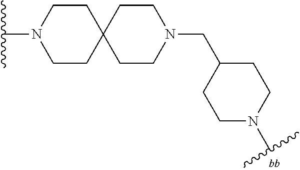 [SMILES: *N1CCC(CN2CCC3(CCN(*)CC3)CC2)CC1]

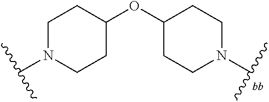 [SMILES: *N1CCC(OC2CCN(*)CC2)CC1]

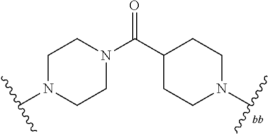 [SMILES: *N1CCC(C(=O)N2CCN(*)CC2)CC1]

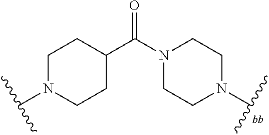 [SMILES: *N1CCC(C(=O)N2CCN(*)CC2)CC1]

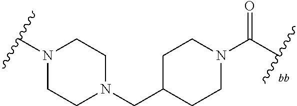 [SMILES: *C(=O)N1CCC(CN2CCN(*)CC2)CC1]

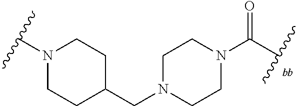 [SMILES: *C(=O)N1CCN(CC2CCN(*)CC2)CC1]

In some embodiments, the compounds of Formula (I-aa) are compounds of Formula (I-aa-1):

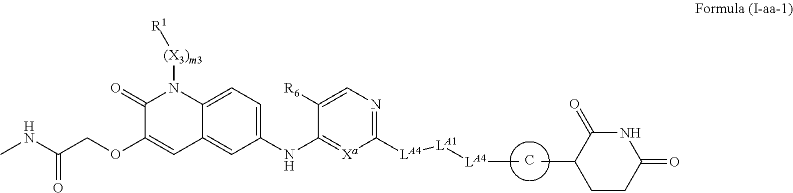

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

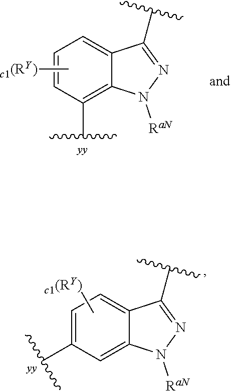

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-1), the -L^{A4}-L^{A1}-L^{A4}- moiety is selected from the group consisting of the moieties delineated in Table L-I-a-1, wherein bb represents the point of attachment to Ring C.

TABLE L-I-a-1

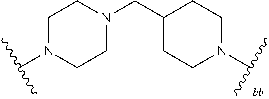 [SMILES: *N1CCC(CN2CCN(*)CC2)CC1]

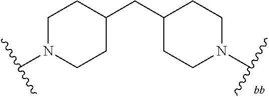 [SMILES: *N1CCC(CC2CCN(*)CC2)CC1]

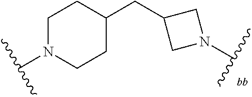 [SMILES: *N1CCC(CC2CN(*)C2)CC1]

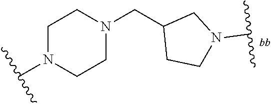 [SMILES: *N1CCN(CC2CCN(*)C2)CC1]

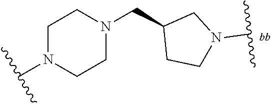 [SMILES: *N1CCN(C[C@@H]2CCN(*)C2)CC1]

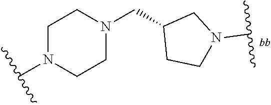 [SMILES: *N1CCN(C[C@H]2CCN(*)C2)CC1]

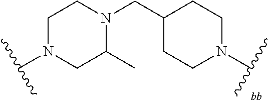 [SMILES: *N1CCC(CN2CCN(*)CC2C)CC1]

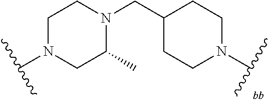 [SMILES: *N1CCC(CN2CCN(*)C[C@H]2C)CC1]

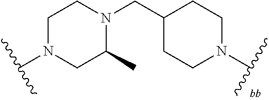 [SMILES: *N1CCC(CN2CCN(*)C[C@@H]2C)CC1]

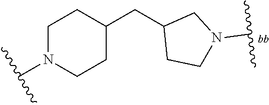 [SMILES: *N1CCC(CC2CCN(*)C2)CC1]

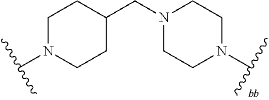 [SMILES: *N1CCC(CN2CCN(*)CC2)CC1]

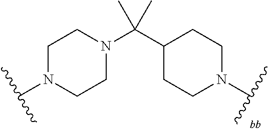 [SMILES: *N1CCC(C(C)(C)N2CCN(*)CC2)CC1]

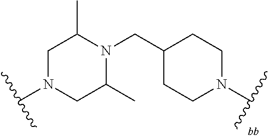 [SMILES: *N1CCC(CN2C(C)CN(*)CC2C)CC1]

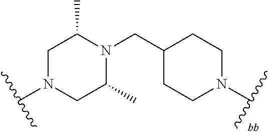 [SMILES: *N1CCC(CN2[C@H](C)CN(*)C[C@@H]2C)CC1]

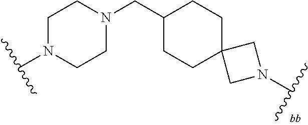 [SMILES: *N1CCN(CC2CCC3(CC2)CN(*)C3)CC1]

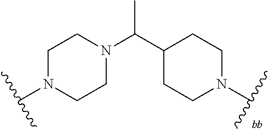 [SMILES: *N1CCC(C(C)N2CCN(*)CC2)CC1]

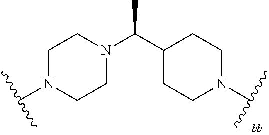 [SMILES: *N1CCC([C@H](C)N2CCN(*)CC2)CC1]

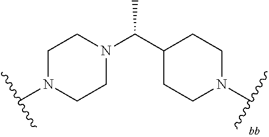 [SMILES: *N1CCC([C@@H](C)N2CCN(*)CC2)CC1]

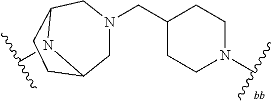 [SMILES: *N1CCC(CN2CC3CCC(C2)N3*)CC1]

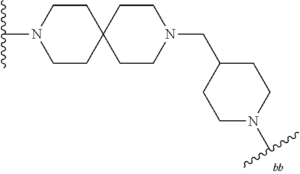 [SMILES: *N1CCC(CN2CCC3(CCN(*)CC3)CC2)CC1]

In some embodiments, the compounds of Formula (I-aa) are compounds of Formula (I-aa-2):

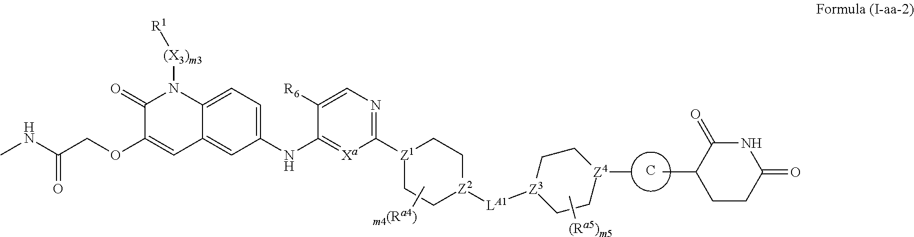

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

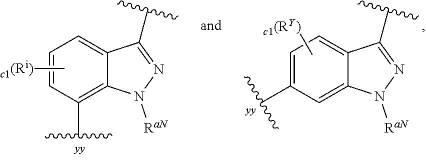

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5},
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

In some embodiments of Formula (I-aa-2), Z¹ is N. In some embodiments of Formula (I-aa-2), Z¹ is N; and Z² is CH or CR^{a4}. In some embodiments of Formula (I-aa-2), Z¹ is N; Z² is CH or CR^{a4}; and Z³ is N.

In some embodiments of Formula (I-aa-2), Z¹ is N; Z² is CH or CR^{a4}; Z³ is N; and Z⁴ is CH or CR^{a5}.

In some embodiments of Formula (I-aa-2), Z¹ is N; Z² is CH; Z³ is N; Z⁴ is CH; and m4 and m5 are both 0. In some embodiments of Formula (I-aa-2), Z¹ is N; Z² is CH; Z³ is N; Z⁴ is CH; one of m4 and m5 is 1; and the other of m4 and m5 is 0. In some embodiments of Formula (I-aa-2), Z¹ is N; Z² is CH; Z³ is N; Z⁴ is CH; one of m4 and m5 is 1; the other of m4 and m5 is 0; and the R^{a4} or R^{a5} when present is methyl. In some embodiments of Formula (I-aa-2), Z¹ is N; Z² is CH; Z³ is N; Z⁴ is CH; m4 is 1; and m5 is 0. In some embodiments of Formula (I-aa-2), Z¹ is N; Z² is CH; Z³ is N; Z⁴ is CH; m4 is 1; m5 is 0; and R^{a4} is methyl.

In some embodiments of Formula (I-aa-2), Z¹ is N; Z² is CH; Z³ is N; and Z⁴ is CR^{a5} (e.g., CF). In some embodiments of Formula (I-aa-2), Z¹ is N; Z² is CH; Z³ is N; Z⁴ is CF; and m4 and m5 are both 0.

In some embodiments of Formula (I-aa-2), Z¹ is N; Z² is CH or CR^{a4}; Z³ is N; and Z⁴ is N. In some embodiments of Formula (I-aa-2), Z¹ is N; Z² is CH; Z³ is N; Z⁴ is N; and m4 and m5 are both 0. In some embodiments of Formula (I-aa-2), Z¹ is N; Z² is CH; Z³ is N; Z⁴ is N; one of m4 and m5 is 1; and the other of m4 and m5 is 0. In some embodiments of Formula (I-aa-2), Z¹ is N; Z² is CH; Z³ is N; Z⁴ is N; one of m4 and m5 is 1; the other of m4 and m5 is 0; and the R^{a4} or R^{a5} when present is methyl. In some embodiments of Formula (I-aa-2), Z¹ is N; Z² is CH; Z³ is N; Z⁴ is N; m4 is 1; m5 is 0. In some embodiments, R^{a4} is methyl.

In some embodiments of Formula (I-aa-2), the

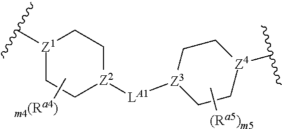

moiety is selected from the group consisting of the moieties delineated in Table L-I-a-2, wherein bb represents the point of attachment to Ring C.

TABLE L-I-a-2

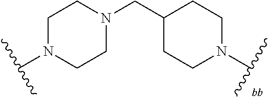 [SMILES: *N1CCC(CN2CCN(*)CC2)CC1]

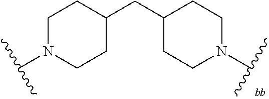 [SMILES: *N1CCC(CC2CCN(*)CC2)CC1]

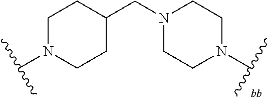 [SMILES: *N1CCC(CN2CCN(*)CC2)CC1]

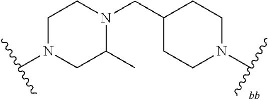 [SMILES: *N1CCC(CN2CCN(*)CC2C)CC1]

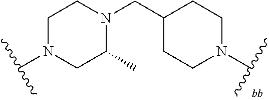 [SMILES: *N1CCC(CN2CCN(*)C[C@H]2C)CC1]

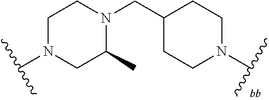 [SMILES: *N1CCC(CN2CCN(*)C[C@@H]2C)CC1]

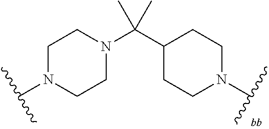 [SMILES: *N1CCC(C(C)(C)N2CCN(*)CC2)CC1]

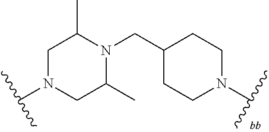 [SMILES: *N1CCC(CN2C(C)CN(*)CC2C)CC1]

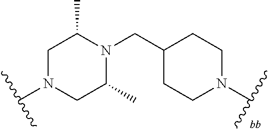 [SMILES: *N1CCC(CN2[C@H](C)CN(*)C[C@@H]2C)CC1]

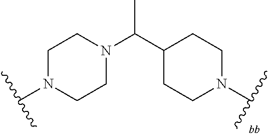 [SMILES: *N1CCC(C(C)N2CCN(*)CC2)CC1]

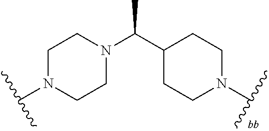 [SMILES: *N1CCC([C@H](C)N2CCN(*)CC2)CC1]

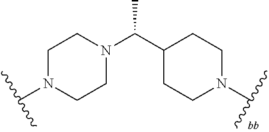 [SMILES: *N1CCC([C@@H](C)N2CCN(*)CC2)CC1]

In some embodiments, the compounds of Formula (I-aa) are compounds of Formula (I-aa-3):

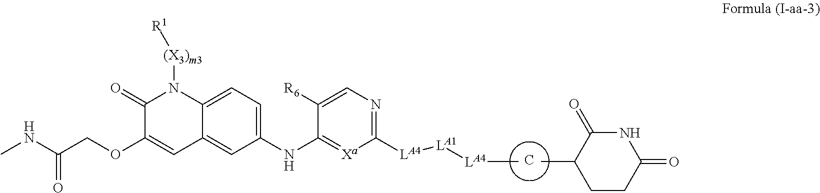

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

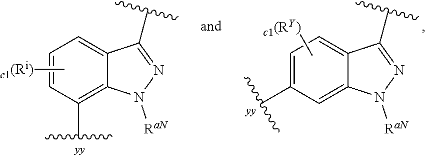

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A1} is CH₂, CHMe, or CMe₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-3), each L^{A4} contains 1-2 ring nitrogen atoms and no additional ring heteroatom.

In some embodiments of Formula (I-aa-3), one L^{A4} is a monocyclic 4-6 membered nitrogen-containing heterocyclylene optionally substituted with 1-3 R^{a}; and the other L^{A4} is a bicyclic spirocyclic 6-12 (e.g., 7, 9, or 11) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-aa-3), one L^{A4} is a monocyclic 4-6 membered nitrogen-containing heterocyclylene optionally substituted with 1-3 R^{a}; and the other L^{A4} is a bicyclic bridged 6-12 (e.g., 7, 8, or 9) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-aa-3), one L^{A4} is a monocyclic 4-6 membered nitrogen-containing heterocyclylene optionally substituted with 1-3 R^{a}; and the other L^{A4} is a bicyclic fused 6-12 (e.g., 6) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-aa-3), the -L^{A4}-L^{A1}-L^{A4}- moiety is selected from the group consisting of the moieties delineated in Table L-I-a-3, wherein bb represents the point of attachment to Ring C.

TABLE L-I-a-3

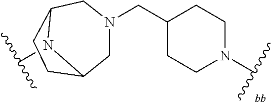 [SMILES: *N1CCC(CN2CC3CCC(C2)N3*)CC1]

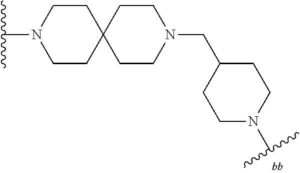 [SMILES: *N1CCC(CN2CCC3(CCN(*)CC3)CC2)CC1]

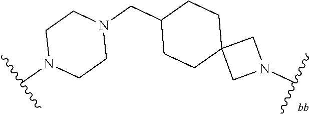 [SMILES: *N1CCN(CC2CCC3(CC2)CN(*)C3)CC1]

In some embodiments, the compounds of Formula (I-aa) are compounds of Formula (I-aa-4):

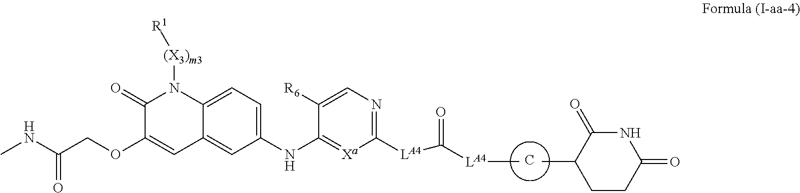

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

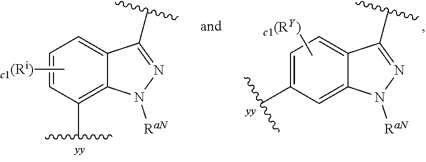

wherein: c1 is 0 or 1, R^{Y} is selected from the group consisting of halo (e.g., —F) and C₁₋₃ alkyl optionally substituted with 1-3 F, and R^{aN} is C₁₋₃ alkyl; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-aa-4), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene and piperidinylene.

In some embodiments of Formula (I-aa-4), the -L^{A4}-C(═O)-L^{A4}- moiety is selected from the group consisting of the moieties delineated in Table L-I-a-4, wherein bb represents the point of attachment to Ring C.

TABLE L-I-a-4

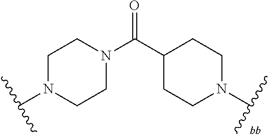 [SMILES: *N1CCC(C(=O)N2CCN(*)CC2)CC1]

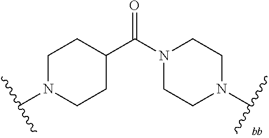 [SMILES: *N1CCC(C(=O)N2CCN(*)CC2)CC1]

In some embodiments, the compounds are compounds of Formula (I-a):

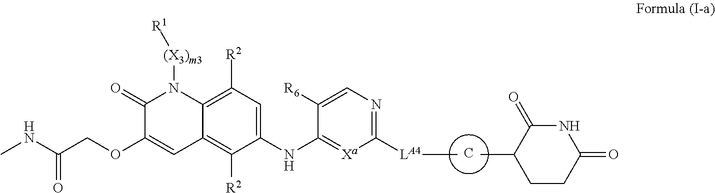

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

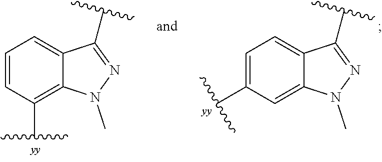 [SMILES: *c1nn(C)c2c(*)cccc12 ; *c1ccc2c(*)nn(C)c2c1]

L is selected from the group consisting of:
-L^{A4}-L^{A1}-L^{A4}-_{bb};
-L^{A4}-O-L^{A4}-_{bb};
-L^{A4}-C(═O)-L^{A4}-_{bb}; and
-L^{A4}-L^{A1}-L^{A4}-C(═O)-_{bb},
wherein bb represents the point of attachment to Ring C;
L^{A1} is CH₂, CHMe, or CMe₂; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a), each L^{A4} is independently a monocyclic 4-6 membered nitrogen-containing heterocyclylene optionally substituted with 1-3 R^{a},
wherein each L^{A4} contains 1-2 ring nitrogen atoms and no additional ring heteroatoms.

In some embodiments of Formula (I-a), one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

In some embodiments of Formula (I-a), one L^{A4} is a monocyclic 4-6 membered nitrogen-containing heterocyclylene optionally substituted with 1-3 R^{a}; and
the other L^{A4} is a bicyclic bridged 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.

In some embodiments of Formula (I-a), L is -L^{A4}-L^{A1}-L^{A4}-_{bb}; and L^{A1} is CH₂ or CHMe.

In some embodiments of Formula (I-a), L is selected from the group consisting of the moieties delineated in Table L1-a.

In some embodiments of Formula (I-a), L is selected from the group consisting of the moieties delineated in Table L-I-a (supra), wherein bb represents the point of attachment to Ring C.

In some embodiments, the compounds of Formula (I-a) are compounds of Formula (I-a-1):

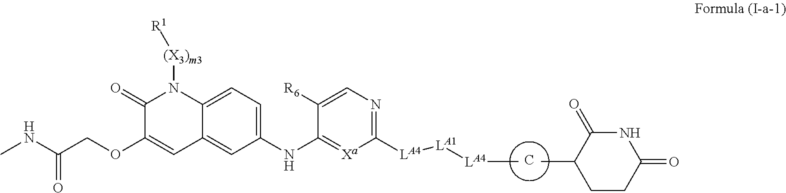

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

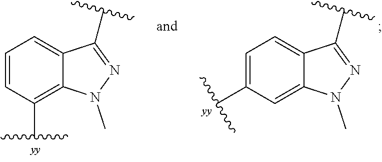 [SMILES: *c1nn(C)c2c(*)cccc12 ; *c1ccc2c(*)nn(C)c2c1]

L^{A1} is CH₂, CHMe, or CMe₂; and
each L^{A4} is independently a monocyclic 4-6 membered nitrogen-containing heterocyclylene optionally substituted with 1-3 R^{a}, wherein:
each L^{A4} contains 1-2 ring nitrogen atoms and no additional ring heteroatoms, and
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-1), the -L^{A4}-L^{A1}-L^{A4}- moiety is selected from the group consisting of the moieties delineated in Table L-I-a-1 (supra), wherein bb represents the point of attachment to Ring C.

In some embodiments, the compounds of Formula (I-a) are compounds of Formula (I-a-2):

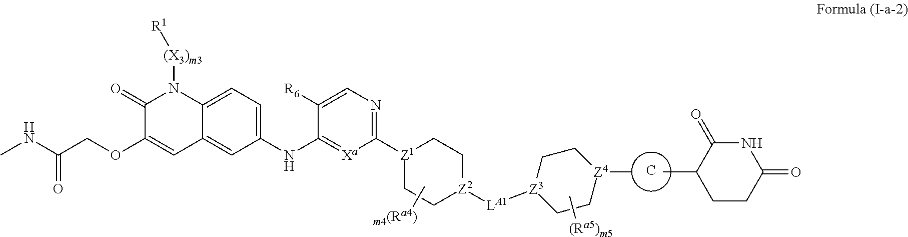

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

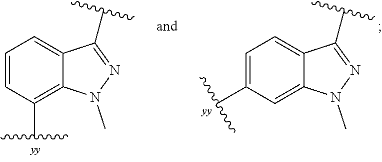 [SMILES: *c1nn(C)c2c(*)cccc12 ; *c1ccc2c(*)nn(C)c2c1]

L^{A1} is CH₂, CHMe, or CMe₂;
Z¹ and Z² are independently selected from the group consisting of: CH, CR^{a4}, and N;
Z³ and Z⁴ are independently selected from the group consisting of: CH, CR^{a5}, and N,
provided that at least one of Z¹ and Z² is N; at least one of Z³ and Z⁴ is N; and when Z² is N, then Z³ is CH or CR^{a5},
m4 and m5 are independently selected from the group consisting of: 0, 1, and 2; and
each R^{a4} and R^{a5} is independently selected from the group consisting of: —F, CN, C₁₋₃ alkoxy, OH, and C₁₋₃ alkyl optionally substituted with 1-3 F.

In some embodiments of Formula (I-a-2), Z¹ is N. In some embodiments of Formula (I-a-2), Z¹ is N; and Z² is CH or CR^{a4}. In some embodiments of Formula (I-a-2), Z¹ is N; Z² is CH or CR^{a4}, and Z³ is N.

In some embodiments of Formula (I-a-2), Z¹ is N; Z² is CH or CR^{a4}; Z³ is N; and Z⁴ is CH or CR^{a5}.

In some embodiments of Formula (I-a-2), Z¹ is N; Z² is CH; Z³ is N; Z⁴ is CH; and m4 and m5 are both 0. In some embodiments of Formula (I-a-2), Z¹ is N; Z² is CH; Z³ is N; Z⁴ is CH; one of m4 and m5 is 1; and the other of m4 and m5 is 0. In some embodiments of Formula (I-a-2), Z¹ is N; Z² is CH; Z³ is N; Z⁴ is CH; one of m4 and m5 is 1; the other of m4 and m5 is 0; and the R^{a4} or R^{a5} when present is methyl. In some embodiments of Formula (I-a-2), Z¹ is N; Z² is CH; Z³ is N; Z⁴ is CH; m4 is 1; and m5 is 0. In some embodiments of Formula (I-a-2), Z¹ is N; Z² is CH; Z³ is N; Z⁴ is CH; m4 is 1; m5 is 0; and R^{a4} is methyl.

In some embodiments of Formula (I-a-2), Z¹ is N; Z² is CH; Z³ is N; and Z⁴ is CR^{a5} (e.g., CF). In some embodiments of Formula (I-a-2), Z¹ is N; Z² is CH; Z³ is N; Z⁴ is CF; and m4 and m5 are both 0.

In some embodiments of Formula (I-a-2), Z¹ is N; Z² is CH or CR^{a4}; Z³ is N; and Z⁴ is N. In some embodiments of Formula (I-a-2), Z¹ is N; Z² is CH; Z³ is N; Z⁴ is N; and m4 and m5 are both 0. In some embodiments of Formula (I-a-2), Z¹ is N; Z² is CH; Z³ is N; ZA is N; one of m4 and m5 is 1; and the other of m4 and m5 is 0. In some embodiments of Formula (I-a-2), Z¹ is N; Z² is CH; Z³ is N; Z⁴ is N; one of m4 and m5 is 1; the other of m4 and m5 is 0; and the R^{a4} or R^{a5} when present is methyl. In some embodiments of Formula (I-a-2), Z¹ is N; Z² is CH; Z³ is N; Z⁴ is N; m4 is 1; m5 is 0. In some embodiments, R^{a4} is methyl.

In some embodiments of Formula (I-a-2), the

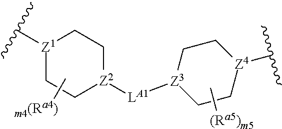

moiety is selected from the group consisting of the moieties delineated in Table L-I-a-2 (supra), wherein bb represents the point of attachment to Ring C.

In some embodiments, the compounds of Formula (I-a) are compounds of Formula (I-a-3):

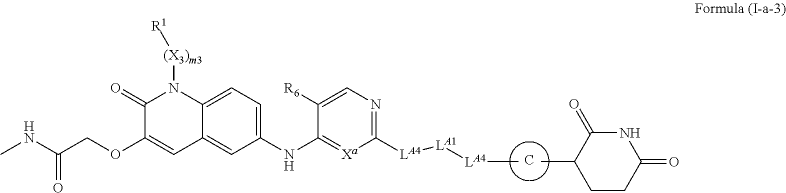

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

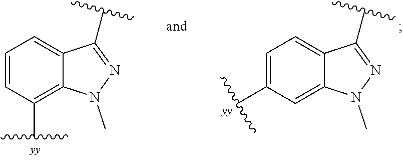 [SMILES: *c1nn(C)c2c(*)cccc12 ; *c1ccc2c(*)nn(C)c2c1]

L^{A1} is CH₂, CHMe, or CMe₂; and
one L^{A4} is a monocyclic 4-6 membered nitrogen-containing heterocyclylene optionally substituted with 1-3 R^{a}; and
the other L^{A4} is a bicyclic 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-3), each L^{A4} contains 1-2 ring nitrogen atoms and no additional ring heteroatom.

In some embodiments of Formula (I-a-3), one L^{A4} is a monocyclic 4-6 membered nitrogen-containing heterocyclylene optionally substituted with 1-3 R^{a}; and the other L^{A4} is a bicyclic spirocyclic 6-12 (e.g., 7, 9, or 11) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-a-3), one L^{A4} is a monocyclic 4-6 membered nitrogen-containing heterocyclylene optionally substituted with 1-3 R^{a}; and the other L^{A4} is a bicyclic bridged 6-12 (e.g., 7, 8, or 9) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-a-3), one L^{A4} is a monocyclic 4-6 membered nitrogen-containing heterocyclylene optionally substituted with 1-3 R^{a}; and the other L^{A4} is a bicyclic fused 6-12 (e.g., 6) membered nitrogen-containing heterocyclylene optionally substituted with 1-3 R^{a}. In some embodiments, each L^{A4} contains 1-2 ring nitrogen atoms and no additional ring heteroatom.

In some embodiments of Formula (I-a-3), the -L^{A4}-L^{A1}-L^{A4}- moiety is selected from the group consisting of the moieties delineated in Table L-I-a-3 (supra), wherein bb represents the point of attachment to Ring C.

In some embodiments, the compounds of Formula (I-a) are compounds of Formula (I-a-4):

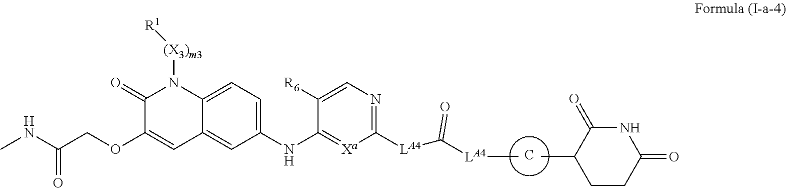

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

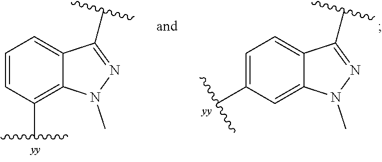 [SMILES: *c1nn(C)c2c(*)cccc12 ; *c1ccc2c(*)nn(C)c2c1]

and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-a-4), each L^{A4} is independently a monocyclic 4-6 membered nitrogen-containing heterocyclylene optionally substituted with 1-3 R^{a}, wherein each L^{A4} contains 1-2 ring nitrogen atoms and no additional ring heteroatoms. For example, each L^{A4} can be independently selected from the group consisting of: piperazinylene and piperidinylene.

In some embodiments of Formula (I-a-4), the -L^{A4}-C(—O)-L^{A4}- moiety is selected from the group consisting of the moieties delineated in Table L-I-a-4 (supra), wherein bb represents the point of attachment to Ring C.

In some embodiments, the compounds of Formula (I) are compounds of Formula (I-bb):

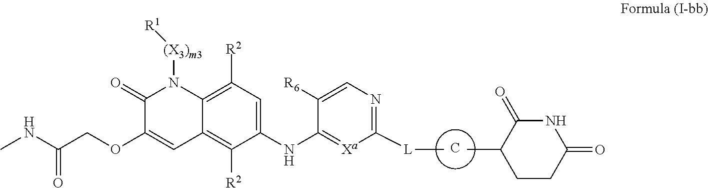

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

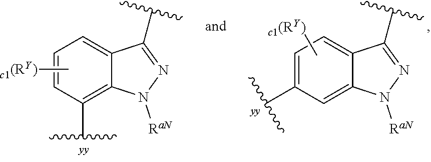

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is -L^{A4}-L^{A3}-_{bb} or -L^{A4}-L^{A1}-L^{A3}-_{bb}, wherein bb represents the point of attachment to Ring C; and
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-bb), L is -L^{A4}-L^{A3}-_{bb}, and L^{A3} is —NH—.

In some embodiments of Formula (I-bb), L^{A4} is a monocyclic 4-6 membered nitrogen-containing heterocyclylene optionally substituted with 1-3 R^{a},
wherein L^{A4} contains 1-2 ring nitrogen atoms and no additional ring heteroatoms.

In some embodiments of Formula (I-bb), L^{A4} is a bicyclic spirocyclic 6-12 (e.g., 6-10) membered nitrogen-containing heterocyclylene optionally substituted with 1-3 R^{a},
wherein L^{A4} contains 1-2 ring nitrogen atoms and no additional ring heteroatoms.

In some embodiments of Formula (I-bb), L is selected from the group consisting of the moieties delineated in Table L2-a.

In some embodiments of Formula (I-bb), L is selected from the group consisting of the moieties delineated in Table L-I-b, wherein bb represents the point of attachment to Ring C.

TABLE L-I-b

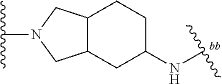 [SMILES: *NC1CCC2CN(*)CC2C1]

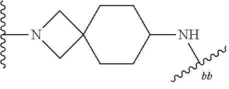 [SMILES: *NC1CCC2(CC1)CN(*)C2]

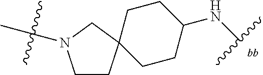 [SMILES: *NC1CCC2(CC1)CCN(*)C2]

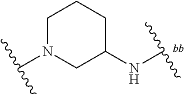 [SMILES: *NC1CCCN(*)C1]

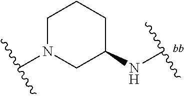 [SMILES: *N[C@@H]1CCCN(*)C1]

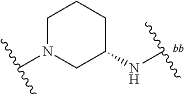 [SMILES: *N[C@H]1CCCN(*)C1]

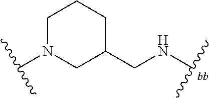 [SMILES: *NCC1CCCN(*)C1]

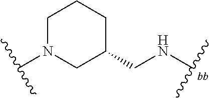 [SMILES: *NC[C@H]1CCCN(*)C1]

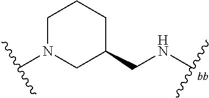 [SMILES: *NC[C@@H]1CCCN(*)C1]

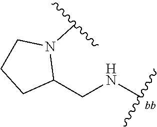 [SMILES: *NCC1CCCN1*]

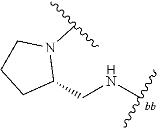 [SMILES: *NC[C@@H]1CCCN1*]

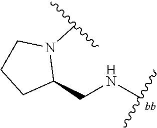 [SMILES: *NC[C@H]1CCCN1*]

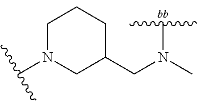 [SMILES: *N(C)CC1CCCN(*)C1]

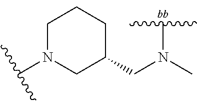 [SMILES: *N(C)C[C@H]1CCCN(*)C1]

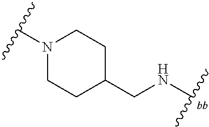 [SMILES: *NCC1CCN(*)CC1]

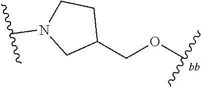 [SMILES: *OCC1CCN(*)C1]

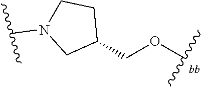 [SMILES: *OC[C@H]1CCN(*)C1]

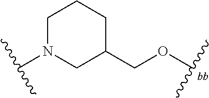 [SMILES: *OCC1CCCN(*)C1]

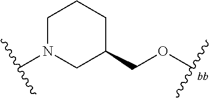 [SMILES: *OC[C@@H]1CCCN(*)C1]

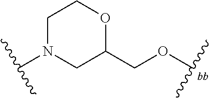 [SMILES: *OCC1CN(*)CCO1]

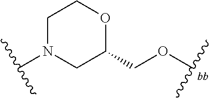 [SMILES: *OC[C@@H]1CN(*)CCO1]

In some embodiments, the compounds of Formula (I-bb) are compounds of Formula (I-bb-1):

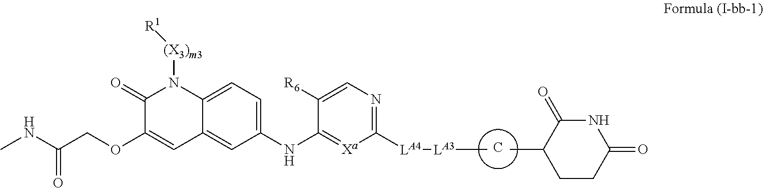

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

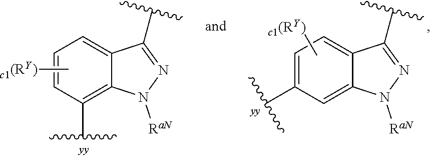

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A4} is 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—, —N(C₁₋₃ alkyl)-, or —O—.

In some embodiments of Formula (I-bb-1), L^{A4} is a bicyclic spirocyclic 6-12 (e.g., 8-10 (e.g., 9)) membered nitrogen-containing heterocyclylene optionally substituted with 1-3 R^{a}, wherein L^{A4} contains 1-2 (e.g., 1) ring nitrogen atoms and no additional ring heteroatoms.

In some embodiments of Formula (I-bb-1), the -L^{A4}-L^{A3}- is selected from the group consisting of the moieties delineated in Table L-I-b-1, wherein bb represents the point of attachment to Ring C.

TABLE L-I-b-1

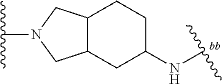 [SMILES: *NC1CCC2CN(*)CC2C1]

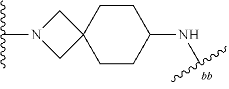 [SMILES: *NC1CCC2(CC1)CN(*)C2]

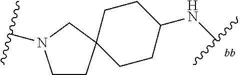 [SMILES: *NC1CCC2(CC1)CCN(*)C2]

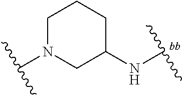 [SMILES: *NC1CCCN(*)C1]

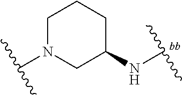 [SMILES: *N[C@@H]1CCCN(*)C1]

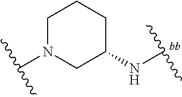 [SMILES: *N[C@H]1CCCN(*)C1]

In some embodiments, the compounds of Formula (I-bb) are compounds of Formula (I-bb-2):

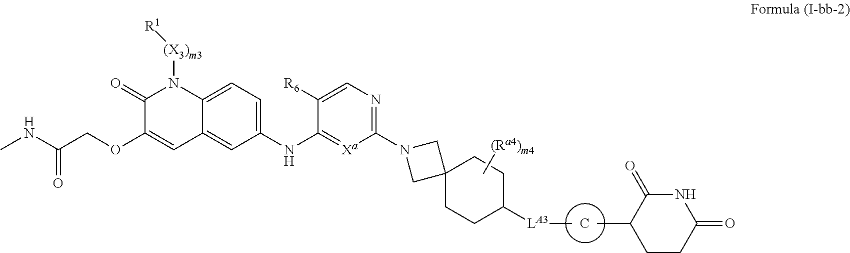

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

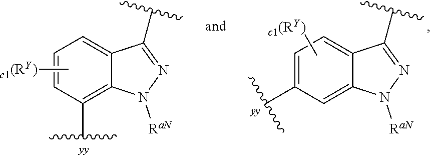

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L^{A3} is —NH—, —N(C₁₋₃ alkyl)-, or —O—;
m4 is selected from the group consisting of: 0, 1, and 2; and
each R^{a4} is independently selected from the group consisting of: —F, CN, C₁₋₃ alkoxy, OH, and C₁₋₃ alkyl optionally substituted with 1-3 F.

In some embodiments of Formula (I-bb-2), m4 is 0 or 1; and R^{a4} when present is methyl.

In some embodiments, the compound is a compound of Formula (I-b):

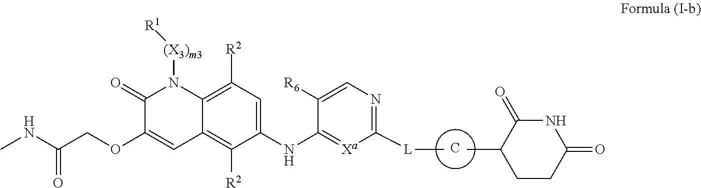

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

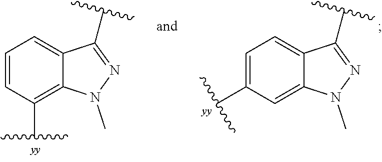 [SMILES: *c1nn(C)c2c(*)cccc12 ; *c1ccc2c(*)nn(C)c2c1]

L is -L^{A4}-L^{A3}-_{bb} or -L^{A4}-L^{A1}-L^{A3}-_{bb}, wherein bb represents the point of attachment to Ring C; and
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In some embodiments of Formula (I-b), L is -L^{A4}-L^{A3}-_{bb}, and L^{A3} is —NH—.

In some embodiments of Formula (I-b), L^{A4} is a monocyclic 4-6 membered nitrogen-containing heterocyclylene optionally substituted with 1-3 R^{a},
wherein L^{A4} contains 1-2 ring nitrogen atoms and no additional ring heteroatoms.

In some embodiments of Formula (I-b), L^{A4} is a bicyclic spirocyclic 6-12 (e.g., 6-10) membered nitrogen-containing heterocyclylene optionally substituted with 1-3 R^{a},
wherein L^{A4} contains 1-2 ring nitrogen atoms and no additional ring heteroatoms.

In some embodiments of Formula (I-b), L is selected from the group consisting of the moieties delineated in Table L2-a.

In some embodiments of Formula (I-b), L is selected from the group consisting of the moieties delineated in Table L-I-b (supra), wherein bb represents the point of attachment to Ring C.

In some embodiments, the compounds of Formula (I-b) are compounds of Formula (I-b-1):

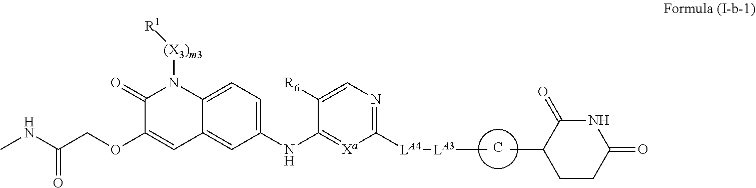

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

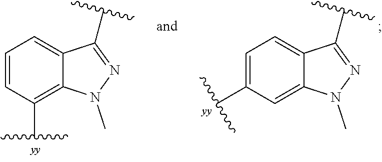 [SMILES: *c1nn(C)c2c(*)cccc12 ; *c1ccc2c(*)nn(C)c2c1]

L^{A4} is 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—, —N(C₁₋₃ alkyl)-, or —O—.

In some embodiments of Formula (I-b-1), L^{A4} is a bicyclic spirocyclic 6-12 (e.g., 8-10 (e.g., 9)) membered nitrogen-containing heterocyclylene optionally substituted with 1-3 R^{a}, wherein L^{A4} contains 1-2 (e.g., 1) ring nitrogen atoms and no additional ring heteroatoms.

In some embodiments of Formula (I-b-1), the -L^{A4}-L^{A3}- is selected from the group consisting of the moieties delineated in Table L-I-b-1 (supra), wherein bb represents the point of attachment to Ring C.

In some embodiments, the compounds of Formula (I-b) are compounds of Formula (I-b-2):

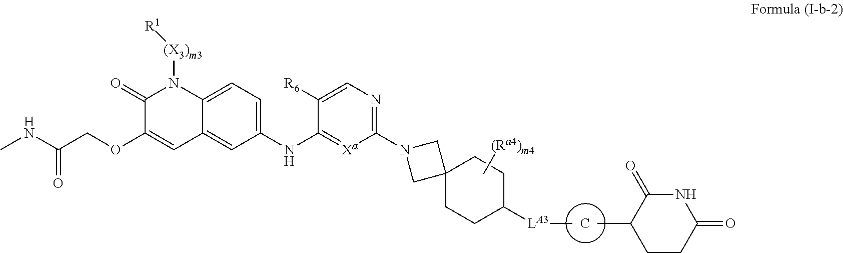

or pharmaceutically acceptable salts thereof, wherein:
X^{a} is N or CH;
R⁶ is —F or —Cl;
m3 is 1, X³ is C₁₋₃ alkylene, and R¹ is H;
Ring C is selected from the group consisting of:

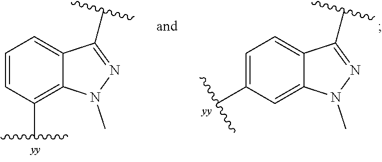 [SMILES: *c1nn(C)c2c(*)cccc12 ; *c1ccc2c(*)nn(C)c2c1]

L^{A3} is —NH—, —N(C₁₋₃ alkyl)-, or —O—;
m4 is selected from the group consisting of: 0, 1, and 2; and
each R^{a4} is independently selected from the group consisting of: —F, CN, C₁₋₃ alkoxy, OH, and C₁₋₃ alkyl optionally substituted with 1-3 F.

In some embodiments of Formula (I-b-2), m4 is 0 or 1; and R^{a4} when present is methyl.

In some embodiments of Formula (I-aa) (e.g., (I-aa-1), (I-aa-2), (I-aa-3), or (I-aa-4)) or (I-bb) (e.g., Formula (I-bb-1)), X^{a} is N.

In some embodiments of Formula (I-aa) (e.g., (I-aa-1), (I-aa-2), (I-aa-3), or (I-aa-4)) or (I-bb) (e.g., Formula (I-bb-1)), X^{a} is CH.

In some embodiments of Formula (I-a) (e.g., (I-a-1), (I-a-2), (I-a-3), or (I-a-4)) or (I-b) (e.g., Formula (I-b-1)), X^{a} is N.

In some embodiments of Formula (I-a) (e.g., (I-a-1), (I-a-2), (I-a-3), or (I-a-4)) or (I-b) (e.g., Formula (I-b-1)), X^{a} is CH.

In some embodiments, the compounds are selected from the group consisting of the compounds in Table C1, or pharmaceutically acceptable salts thereof.

TABLE C1

| No. | Compound Structure |
|---|---|
| 101 | 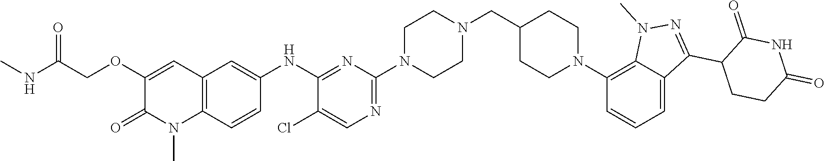 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)ccc2n(C)c1=O] |
| 102 | 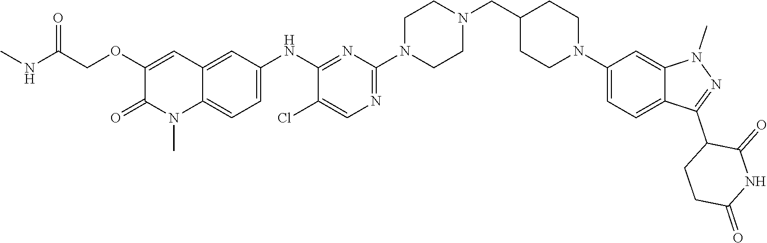 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)ccc2n(C)c1=O] |
| 103 | 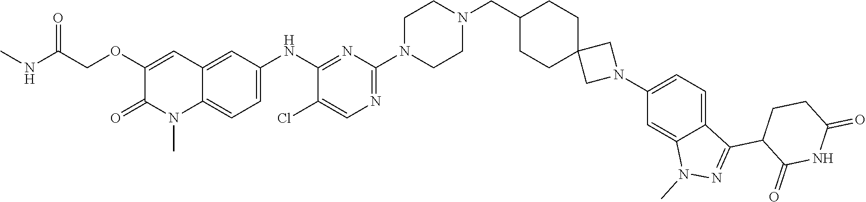 [SMILES: CNC(=O)COc1cc2cc(Nc3nc(N4CCN(CC5CCC6(CC5)CN(c5ccc7c(C8CCC(=O)NC8=O)nn(C)c7c5)C6)CC4)ncc3Cl)ccc2n(C)c1=O] |
| 104 | 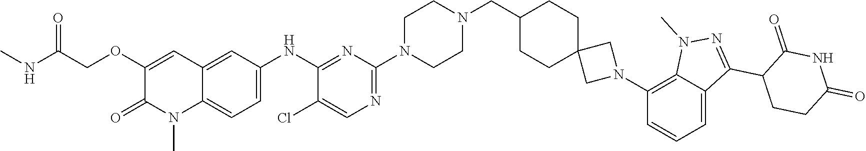 [SMILES: CNC(=O)COc1cc2cc(Nc3nc(N4CCN(CC5CCC6(CC5)CN(c5cccc7c(C8CCC(=O)NC8=O)nn(C)c57)C6)CC4)ncc3Cl)ccc2n(C)c1=O] |
| 105 | 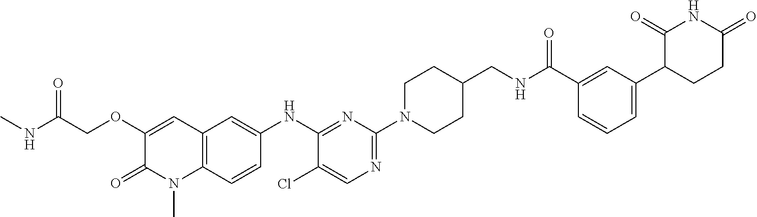 [SMILES: CNC(=O)COc1cc2cc(Nc3nc(N4CCC(CNC(=O)c5cccc(C6CCC(=O)NC6=O)c5)CC4)ncc3Cl)ccc2n(C)c1=O] |
| 106 | 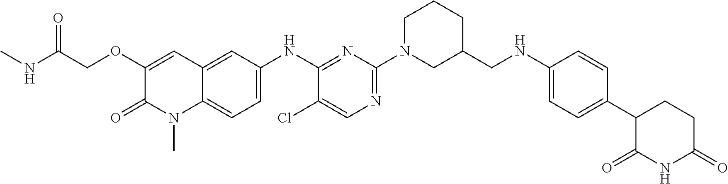 [SMILES: CNC(=O)COc1cc2cc(Nc3nc(N4CCCC(CNc5ccc(C6CCC(=O)NC6=O)cc5)C4)ncc3Cl)ccc2n(C)c1=O] |
| 106a | 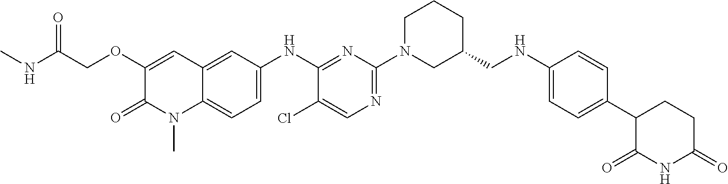 [SMILES: CNC(=O)COc1cc2cc(Nc3nc(N4CCC[C@H](CNc5ccc(C6CCC(=O)NC6=O)cc5)C4)ncc3Cl)ccc2n(C)c1=O] |
| 106b | 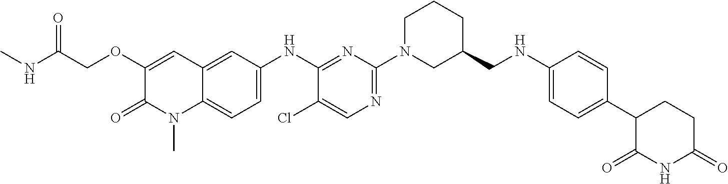 [SMILES: CNC(=O)COc1cc2cc(Nc3nc(N4CCC[C@@H](CNc5ccc(C6CCC(=O)NC6=O)cc5)C4)ncc3Cl)ccc2n(C)c1=O] |
| 107 | 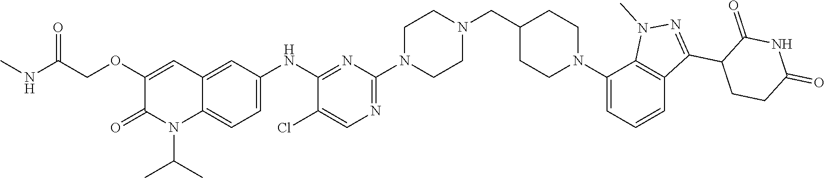 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)ccc2n(C(C)C)c1=O] |
| 108 | 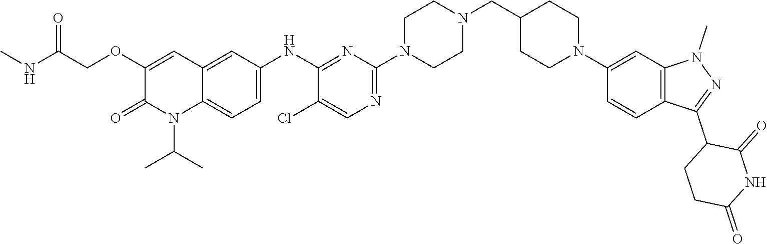 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)ccc2n(C(C)C)c1=O] |
| 260 | 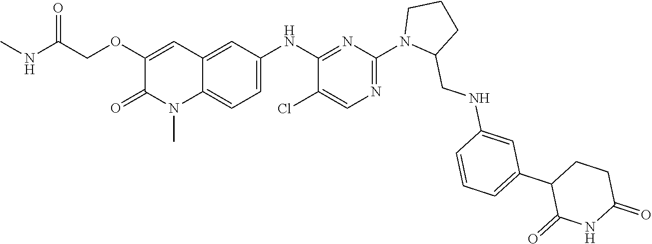 [SMILES: CNC(=O)COc1cc2cc(Nc3nc(N4CCCC4CNc4cccc(C5CCC(=O)NC5=O)c4)ncc3Cl)ccc2n(C)c1=O] |
| 260a | 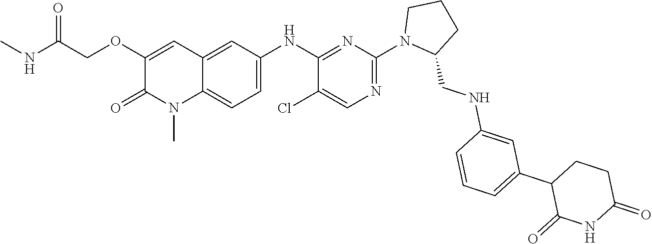 [SMILES: CNC(=O)COc1cc2cc(Nc3nc(N4CCC[C@@H]4CNc4cccc(C5CCC(=O)NC5=O)c4)ncc3Cl)ccc2n(C)c1=O] |
| 260b | 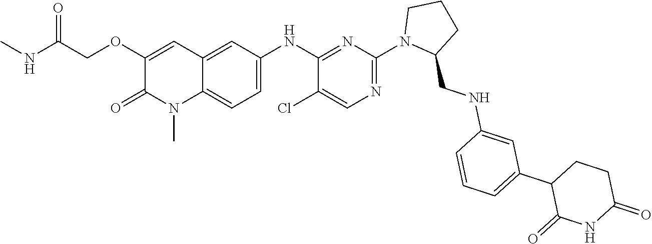 [SMILES: CNC(=O)COc1cc2cc(Nc3nc(N4CCC[C@H]4CNc4cccc(C5CCC(=O)NC5=O)c4)ncc3Cl)ccc2n(C)c1=O] |
| 261 | 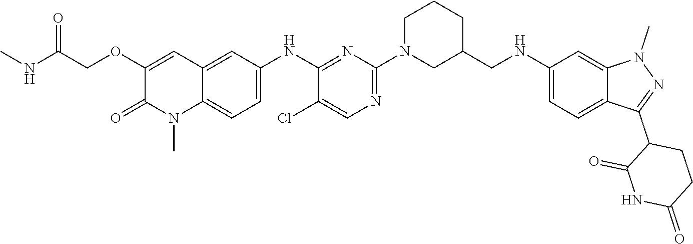 [SMILES: CNC(=O)COc1cc2cc(Nc3nc(N4CCCC(CNc5ccc6c(C7CCC(=O)NC7=O)nn(C)c6c5)C4)ncc3Cl)ccc2n(C)c1=O] |
| 261a | 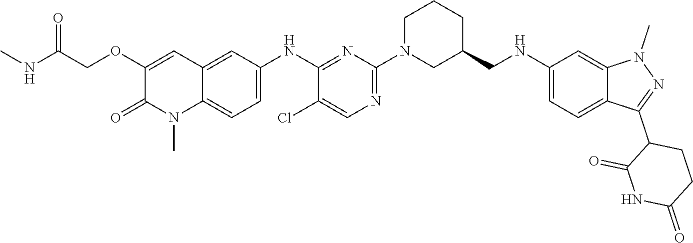 [SMILES: CNC(=O)COc1cc2cc(Nc3nc(N4CCC[C@@H](CNc5ccc6c(C7CCC(=O)NC7=O)nn(C)c6c5)C4)ncc3Cl)ccc2n(C)c1=O] |
| 261b | 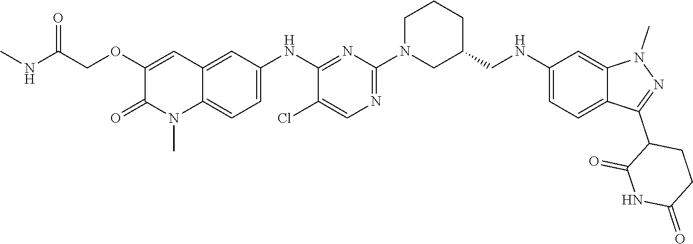 [SMILES: CNC(=O)COc1cc2cc(Nc3nc(N4CCC[C@H](CNc5ccc6c(C7CCC(=O)NC7=O)nn(C)c6c5)C4)ncc3Cl)ccc2n(C)c1=O] |
| 262 | 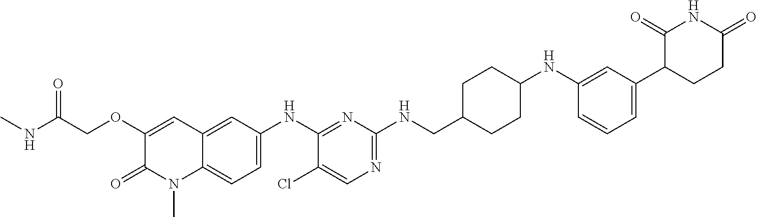 [SMILES: CNC(=O)COc1cc2cc(Nc3nc(NCC4CCC(Nc5cccc(C6CCC(=O)NC6=O)c5)CC4)ncc3Cl)ccc2n(C)c1=O] |
| 262a | 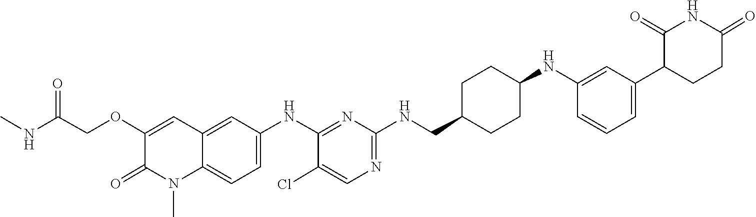 [SMILES: CNC(=O)COc1cc2cc(Nc3nc(NC[C@H]4CC[C@@H](Nc5cccc(C6CCC(=O)NC6=O)c5)CC4)ncc3Cl)ccc2n(C)c1=O] |
| 262b | 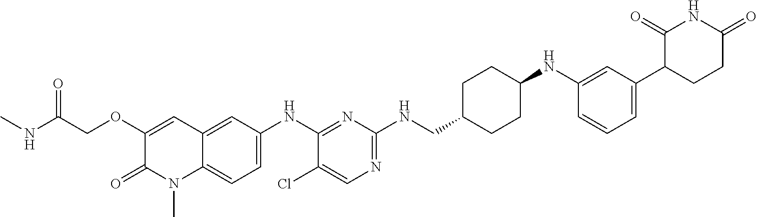 [SMILES: CNC(=O)COc1cc2cc(Nc3nc(NC[C@H]4CC[C@H](Nc5cccc(C6CCC(=O)NC6=O)c5)CC4)ncc3Cl)ccc2n(C)c1=O] |
| 263 | 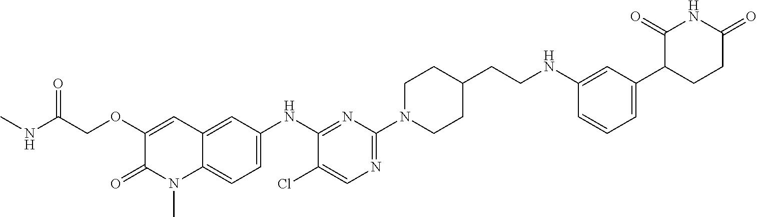 [SMILES: CNC(=O)COc1cc2cc(Nc3nc(N4CCC(CCNc5cccc(C6CCC(=O)NC6=O)c5)CC4)ncc3Cl)ccc2n(C)c1=O] |
| 265 | 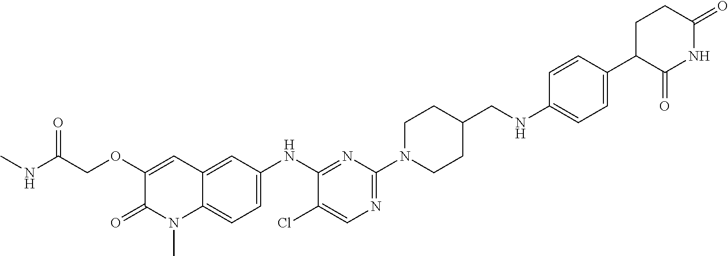 [SMILES: CNC(=O)COc1cc2cc(Nc3nc(N4CCC(CNc5ccc(C6CCC(=O)NC6=O)cc5)CC4)ncc3Cl)ccc2n(C)c1=O] |
| 266 | 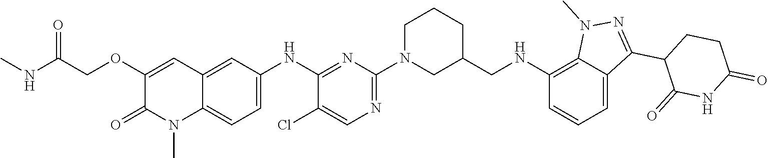 [SMILES: CNC(=O)COc1cc2cc(Nc3nc(N4CCCC(CNc5cccc6c(C7CCC(=O)NC7=O)nn(C)c56)C4)ncc3Cl)ccc2n(C)c1=O] |
| 266a | 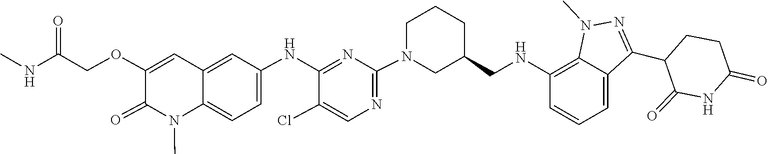 [SMILES: CNC(=O)COc1cc2cc(Nc3nc(N4CCC[C@@H](CNc5cccc6c(C7CCC(=O)NC7=O)nn(C)c56)C4)ncc3Cl)ccc2n(C)c1=O] |
| 266b | 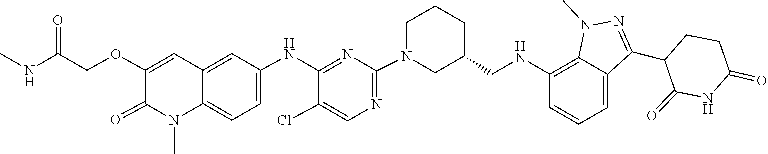 [SMILES: CNC(=O)COc1cc2cc(Nc3nc(N4CCC[C@H](CNc5cccc6c(C7CCC(=O)NC7=O)nn(C)c56)C4)ncc3Cl)ccc2n(C)c1=O] |
| 275 | 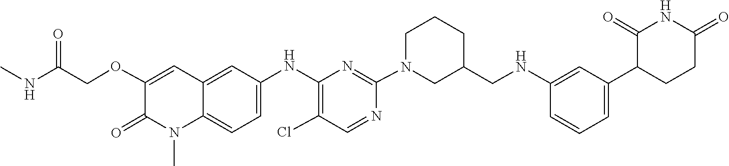 [SMILES: CNC(=O)COc1cc2cc(Nc3nc(N4CCCC(CNc5cccc(C6CCC(=O)NC6=O)c5)C4)ncc3Cl)ccc2n(C)c1=O] |
| 275a | 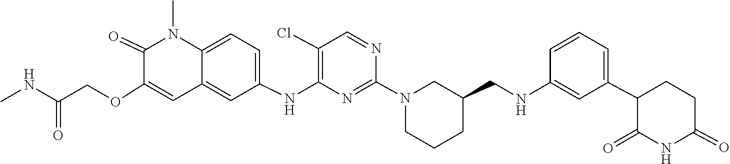 [SMILES: CNC(=O)COc1cc2cc(Nc3nc(N4CCC[C@H](CNc5cccc(C6CCC(=O)NC6=O)c5)C4)ncc3Cl)ccc2n(C)c1=O] |
| 275b | 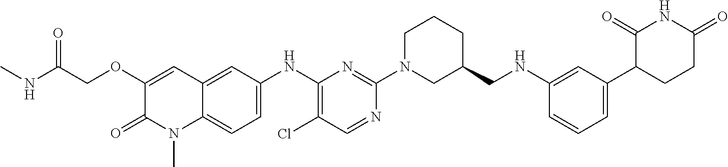 [SMILES: CNC(=O)COc1cc2cc(Nc3nc(N4CCC[C@@H](CNc5cccc(C6CCC(=O)NC6=O)c5)C4)ncc3Cl)ccc2n(C)c1=O] |
| 277 | 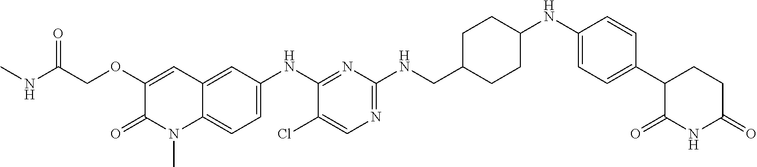 [SMILES: CNC(=O)COc1cc2cc(Nc3nc(NCC4CCC(Nc5ccc(C6CCC(=O)NC6=O)cc5)CC4)ncc3Cl)ccc2n(C)c1=O] |
| 277a | 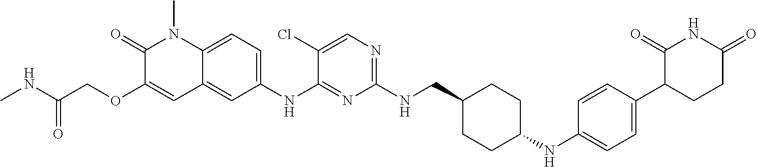 [SMILES: CNC(=O)COc1cc2cc(Nc3nc(NC[C@H]4CC[C@H](Nc5ccc(C6CCC(=O)NC6=O)cc5)CC4)ncc3Cl)ccc2n(C)c1=O] |
| 277b | 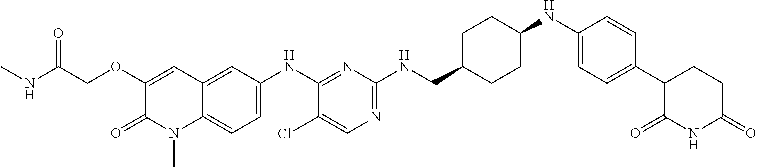 [SMILES: CNC(=O)COc1cc2cc(Nc3nc(NC[C@H]4CC[C@@H](Nc5ccc(C6CCC(=O)NC6=O)cc5)CC4)ncc3Cl)ccc2n(C)c1=O] |
| 279 | 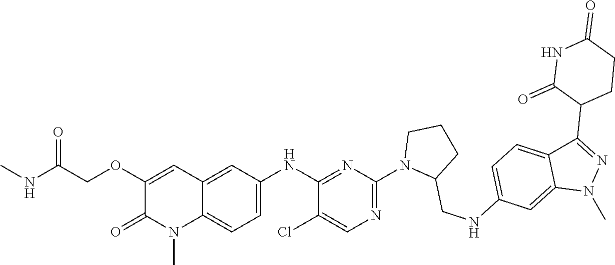 [SMILES: CNC(=O)COc1cc2cc(Nc3nc(N4CCCC4CNc4ccc5c(C6CCC(=O)NC6=O)nn(C)c5c4)ncc3Cl)ccc2n(C)c1=O] |
| 279a | 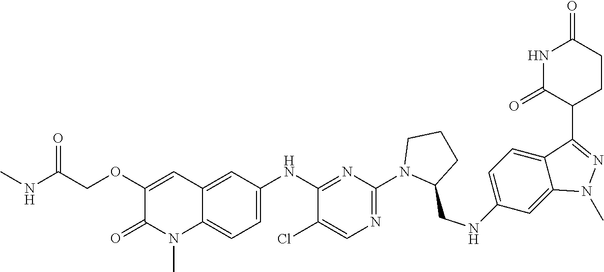 |
| 279b | 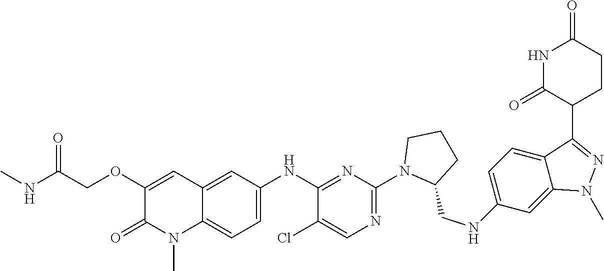 |
| 280 | 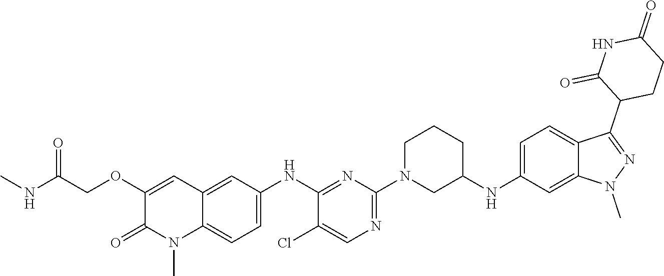 [SMILES: CNC(=O)COc1cc2cc(Nc3nc(N4CCCC(Nc5ccc6c(C7CCC(=O)NC7=O)nn(C)c6c5)C4)ncc3Cl)ccc2n(C)c1=O] |
| 280a | 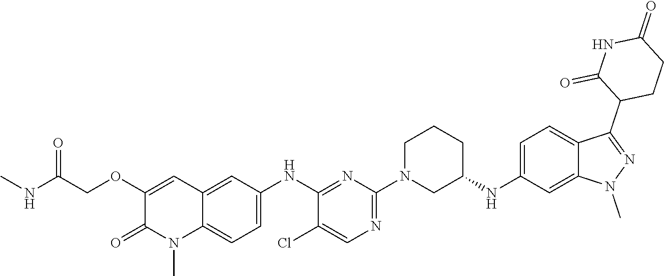 [SMILES: CNC(=O)COc1cc2cc(Nc3nc(N4CCC[C@H](Nc5ccc6c(C7CCC(=O)NC7=O)nn(C)c6c5)C4)ncc3Cl)ccc2n(C)c1=O] |
| 280b | 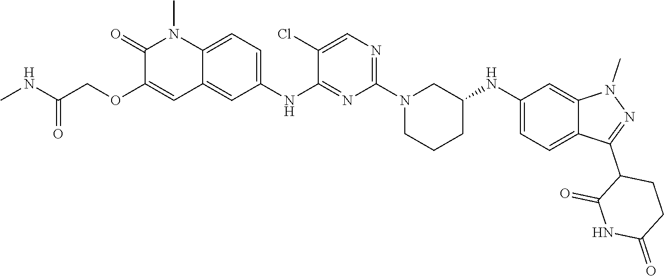 [SMILES: CNC(=O)COc1cc2cc(Nc3nc(N4CCC[C@@H](Nc5ccc6c(C7CCC(=O)NC7=O)nn(C)c6c5)C4)ncc3Cl)ccc2n(C)c1=O] |
| 281 | 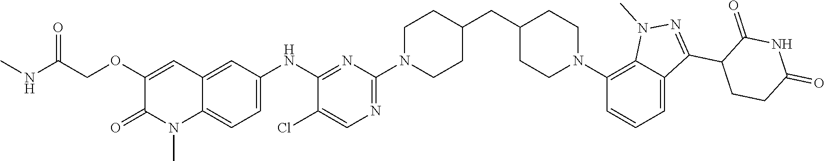 [SMILES: CNC(=O)COc1cc2cc(Nc3nc(N4CCC(CC5CCN(c6cccc7c(C8CCC(=O)NC8=O)nn(C)c67)CC5)CC4)ncc3Cl)ccc2n(C)c1=O] |
| 282 | 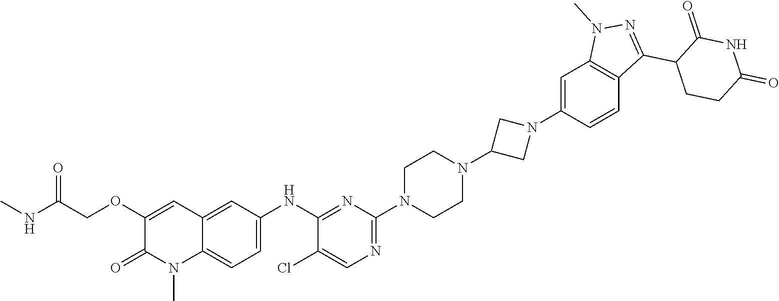 [SMILES: CNC(=O)COc1cc2cc(Nc3nc(N4CCN(C5CN(c6ccc7c(C8CCC(=O)NC8=O)nn(C)c7c6)C5)CC4)ncc3Cl)ccc2n(C)c1=O] |
| 283 | 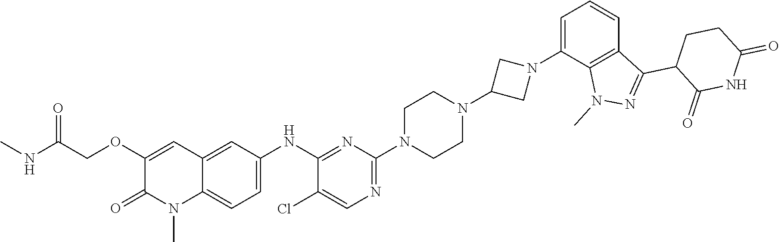 [SMILES: CNC(=O)COc1cc2cc(Nc3nc(N4CCN(C5CN(c6cccc7c(C8CCC(=O)NC8=O)nn(C)c67)C5)CC4)ncc3Cl)ccc2n(C)c1=O] |
| 284 | 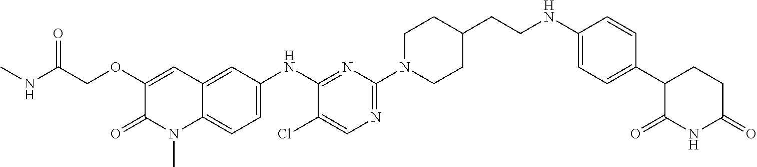 [SMILES: CNC(=O)COc1cc2cc(Nc3nc(N4CCC(CCNc5ccc(C6CCC(=O)NC6=O)cc5)CC4)ncc3Cl)ccc2n(C)c1=O] |
| 286 | 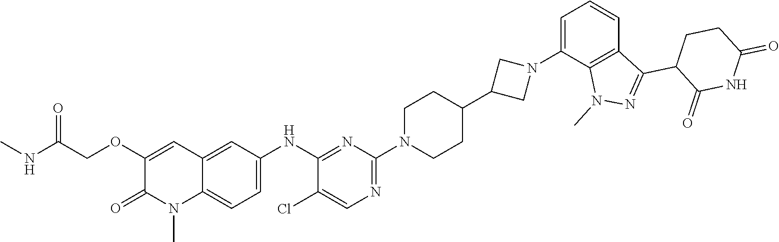 [SMILES: CNC(=O)COc1cc2cc(Nc3nc(N4CCC(C5CN(c6cccc7c(C8CCC(=O)NC8=O)nn(C)c67)C5)CC4)ncc3Cl)ccc2n(C)c1=O] |
| 287 | 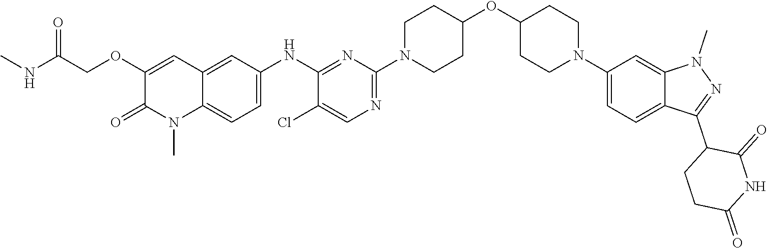 [SMILES: CNC(=O)COc1cc2cc(Nc3nc(N4CCC(OC5CCN(c6ccc7c(C8CCC(=O)NC8=O)nn(C)c7c6)CC5)CC4)ncc3Cl)ccc2n(C)c1=O] |
| 288 | 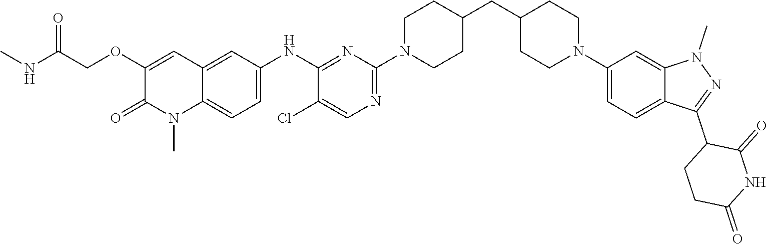 [SMILES: CNC(=O)COc1cc2cc(Nc3nc(N4CCC(CC5CCN(c6ccc7c(C8CCC(=O)NC8=O)nn(C)c7c6)CC5)CC4)ncc3Cl)ccc2n(C)c1=O] |
| 289 | 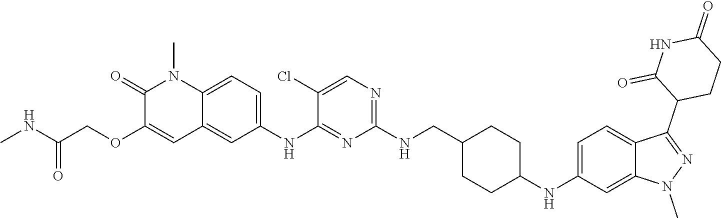 [SMILES: CNC(=O)COc1cc2cc(Nc3nc(NCC4CCC(Nc5ccc6c(C7CCC(=O)NC7=O)nn(C)c6c5)CC4)ncc3Cl)ccc2n(C)c1=O] |
| 289a | 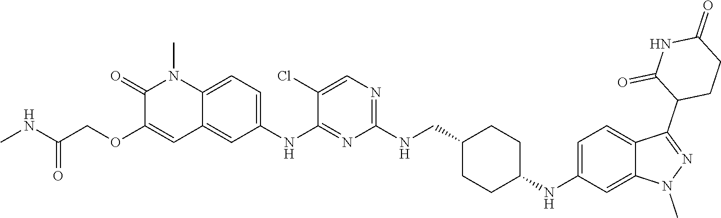 [SMILES: CNC(=O)COc1cc2cc(Nc3nc(NC[C@H]4CC[C@@H](Nc5ccc6c(C7CCC(=O)NC7=O)nn(C)c6c5)CC4)ncc3Cl)ccc2n(C)c1=O] |
| 289b | 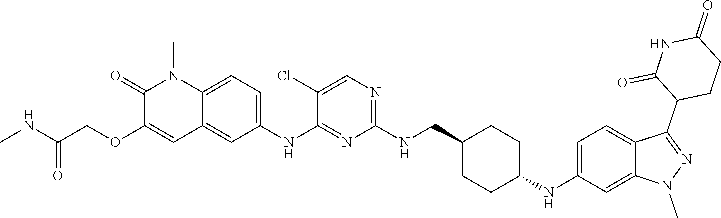 [SMILES: CNC(=O)COc1cc2cc(Nc3nc(NC[C@H]4CC[C@H](Nc5ccc6c(C7CCC(=O)NC7=O)nn(C)c6c5)CC4)ncc3Cl)ccc2n(C)c1=O] |
| 290 | 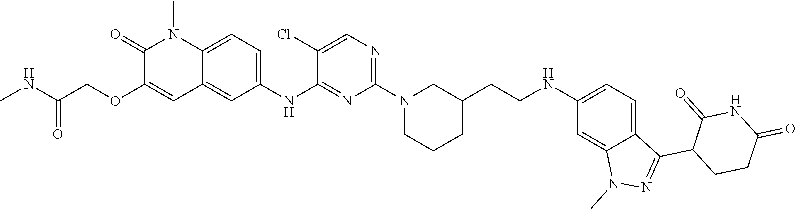 [SMILES: CNC(=O)COc1cc2cc(Nc3nc(N4CCCC(CCNc5ccc6c(C7CCC(=O)NC7=O)nn(C)c6c5)C4)ncc3Cl)ccc2n(C)c1=O] |
| 290a | 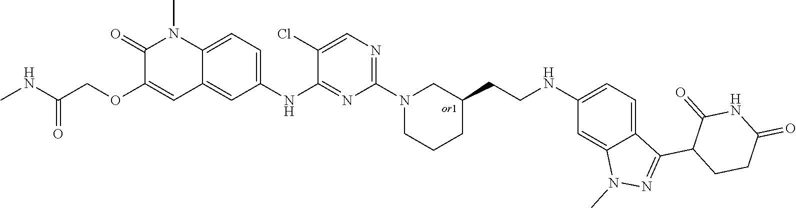 [SMILES: CNC(=O)COc1cc2cc(Nc3nc(N4CCC[C@H](CCNc5ccc6c(C7CCC(=O)NC7=O)nn(C)c6c5)C4)ncc3Cl)ccc2n(C)c1=O] |
| 290b | 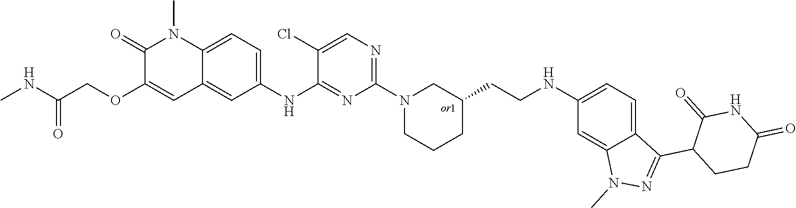 [SMILES: CNC(=O)COc1cc2cc(Nc3nc(N4CCC[C@@H](CCNc5ccc6c(C7CCC(=O)NC7=O)nn(C)c6c5)C4)ncc3Cl)ccc2n(C)c1=O] |
| 292 | 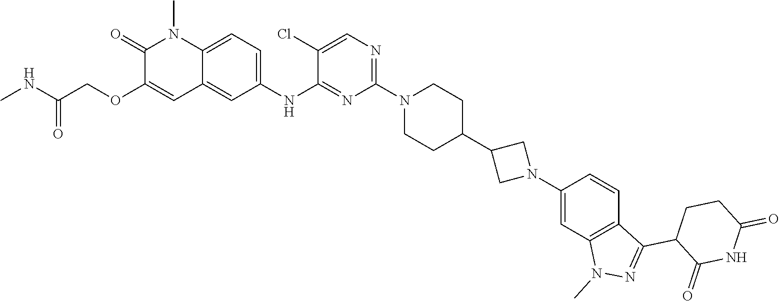 [SMILES: CNC(=O)COc1cc2cc(Nc3nc(N4CCC(C5CN(c6ccc7c(C8CCC(=O)NC8=O)nn(C)c7c6)C5)CC4)ncc3Cl)ccc2n(C)c1=O] |
| 293 | 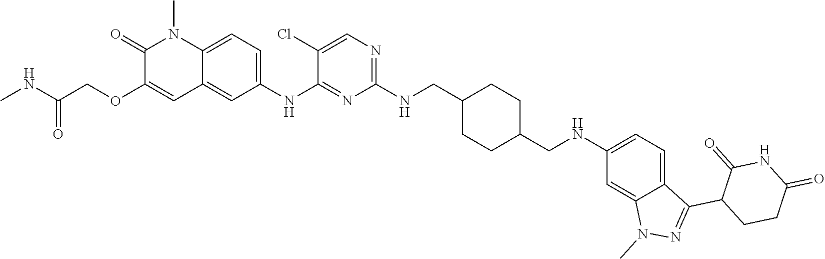 [SMILES: CNC(=O)COc1cc2cc(Nc3nc(NCC4CCC(CNc5ccc6c(C7CCC(=O)NC7=O)nn(C)c6c5)CC4)ncc3Cl)ccc2n(C)c1=O] |
| 293a | 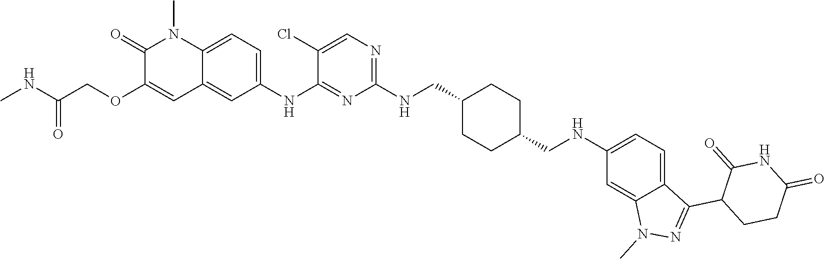 [SMILES: CNC(=O)COc1cc2cc(Nc3nc(NC[C@H]4CC[C@@H](CNc5ccc6c(C7CCC(=O)NC7=O)nn(C)c6c5)CC4)ncc3Cl)ccc2n(C)c1=O] |
| 293b | 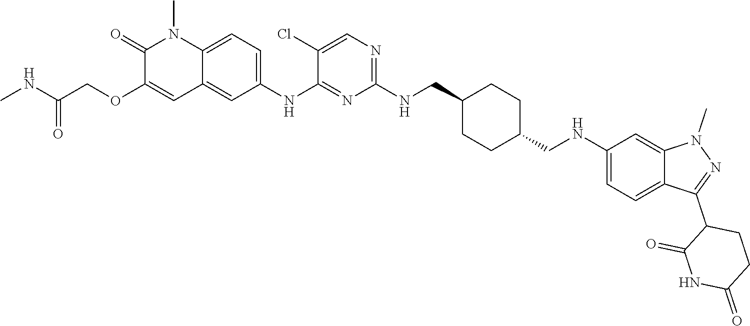 [SMILES: CNC(=O)COc1cc2cc(Nc3nc(NC[C@H]4CC[C@H](CNc5ccc6c(C7CCC(=O)NC7=O)nn(C)c6c5)CC4)ncc3Cl)ccc2n(C)c1=O] |
| 294 | 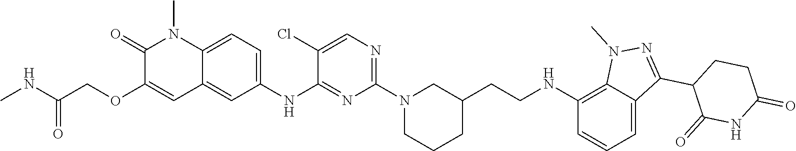 [SMILES: CNC(=O)COc1cc2cc(Nc3nc(N4CCCC(CCNc5cccc6c(C7CCC(=O)NC7=O)nn(C)c56)C4)ncc3Cl)ccc2n(C)c1=O] |
| 294a | 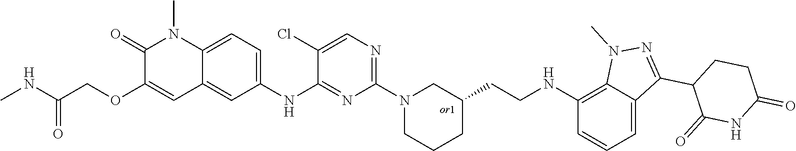 [SMILES: CNC(=O)COc1cc2cc(Nc3nc(N4CCC[C@@H](CCNc5cccc6c(C7CCC(=O)NC7=O)nn(C)c56)C4)ncc3Cl)ccc2n(C)c1=O] |
| 294b | 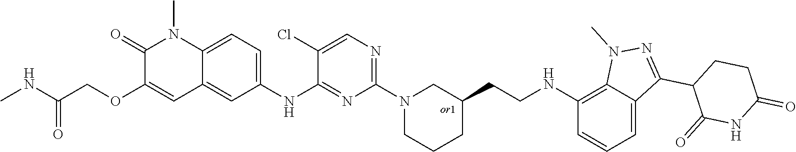 [SMILES: CNC(=O)COc1cc2cc(Nc3nc(N4CCC[C@H](CCNc5cccc6c(C7CCC(=O)NC7=O)nn(C)c56)C4)ncc3Cl)ccc2n(C)c1=O] |
| 295 | 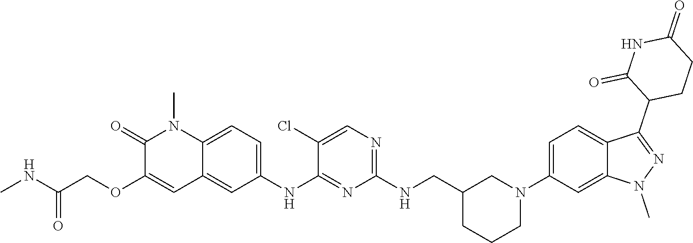 [SMILES: CNC(=O)COc1cc2cc(Nc3nc(NCC4CCCN(c5ccc6c(C7CCC(=O)NC7=O)nn(C)c6c5)C4)ncc3Cl)ccc2n(C)c1=O] |
| 295a | 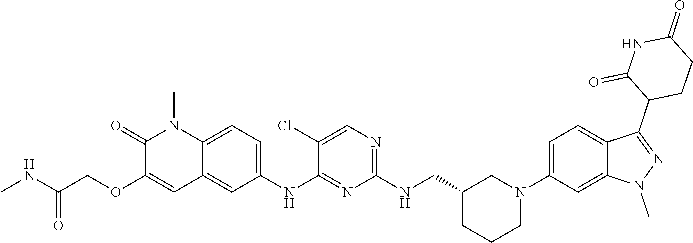 [SMILES: CNC(=O)COc1cc2cc(Nc3nc(NC[C@H]4CCCN(c5ccc6c(C7CCC(=O)NC7=O)nn(C)c6c5)C4)ncc3Cl)ccc2n(C)c1=O] |
| 295b | 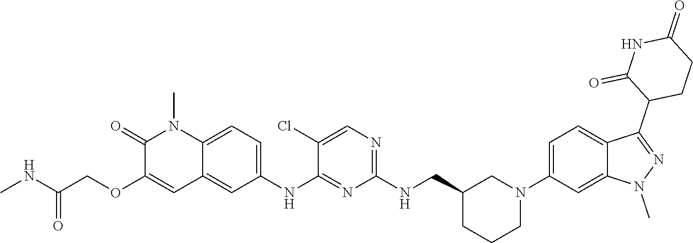 [SMILES: CNC(=O)COc1cc2cc(Nc3nc(NC[C@@H]4CCCN(c5ccc6c(C7CCC(=O)NC7=O)nn(C)c6c5)C4)ncc3Cl)ccc2n(C)c1=O] |
| 296 | 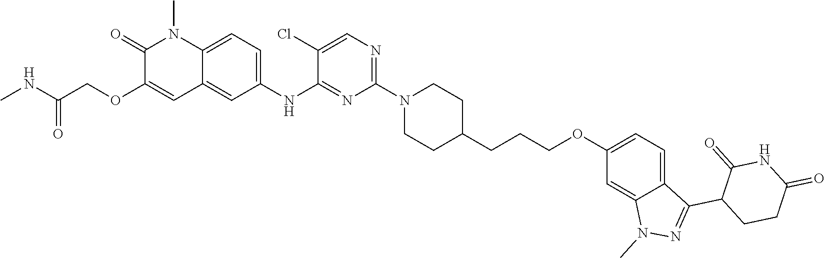 [SMILES: CNC(=O)COc1cc2cc(Nc3nc(N4CCC(CCCOc5ccc6c(C7CCC(=O)NC7=O)nn(C)c6c5)CC4)ncc3Cl)ccc2n(C)c1=O] |
| 297 | 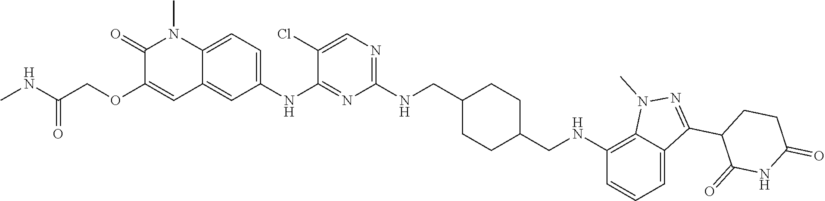 [SMILES: CNC(=O)COc1cc2cc(Nc3nc(NCC4CCC(CNc5cccc6c(C7CCC(=O)NC7=O)nn(C)c56)CC4)ncc3Cl)ccc2n(C)c1=O] |
| 297a | 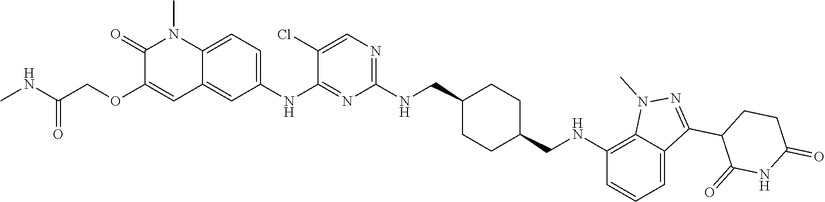 [SMILES: CNC(=O)COc1cc2cc(Nc3nc(NC[C@H]4CC[C@@H](CNc5cccc6c(C7CCC(=O)NC7=O)nn(C)c56)CC4)ncc3Cl)ccc2n(C)c1=O] |
| 297b | 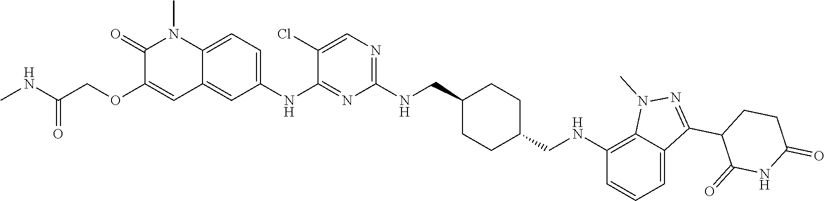 [SMILES: CNC(=O)COc1cc2cc(Nc3nc(NC[C@H]4CC[C@H](CNc5cccc6c(C7CCC(=O)NC7=O)nn(C)c56)CC4)ncc3Cl)ccc2n(C)c1=O] |
| 298 | 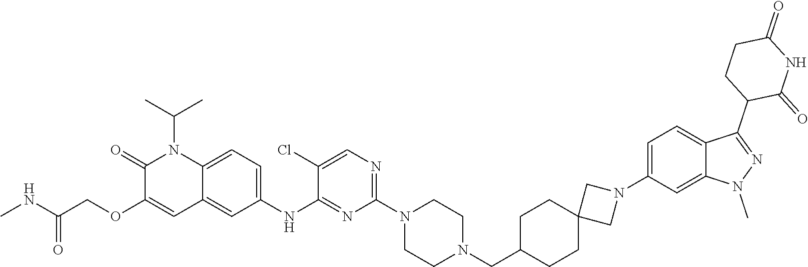 [SMILES: CNC(=O)COc1cc2cc(Nc3nc(N4CCN(CC5CCC6(CC5)CN(c5ccc7c(C8CCC(=O)NC8=O)nn(C)c7c5)C6)CC4)ncc3Cl)ccc2n(C(C)C)c1=O] |
| 299 | 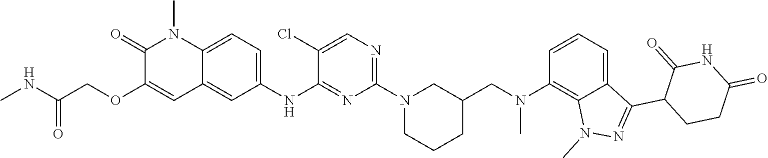 [SMILES: CNC(=O)COc1cc2cc(Nc3nc(N4CCCC(CN(C)c5cccc6c(C7CCC(=O)NC7=O)nn(C)c56)C4)ncc3Cl)ccc2n(C)c1=O] |
| 299a | 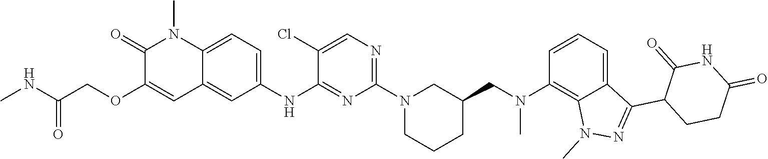 [SMILES: CNC(=O)COc1cc2cc(Nc3nc(N4CCC[C@H](CN(C)c5cccc6c(C7CCC(=O)NC7=O)nn(C)c56)C4)ncc3Cl)ccc2n(C)c1=O] |
| 300 | 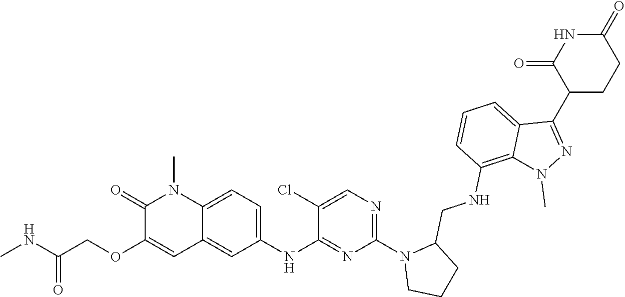 [SMILES: CNC(=O)COc1cc2cc(Nc3nc(N4CCCC4CNc4cccc5c(C6CCC(=O)NC6=O)nn(C)c45)ncc3Cl)ccc2n(C)c1=O] |
| 300a | 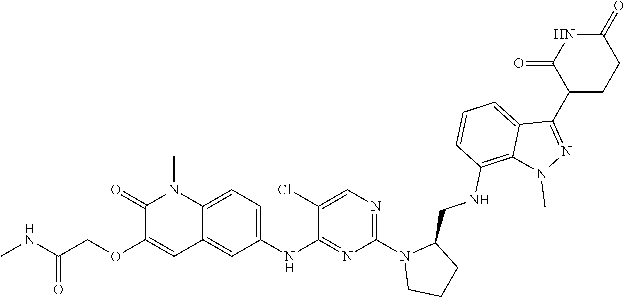 [SMILES: CNC(=O)COc1cc2cc(Nc3nc(N4CCC[C@@H]4CNc4cccc5c(C6CCC(=O)NC6=O)nn(C)c45)ncc3Cl)ccc2n(C)c1=O] |
| 300b | 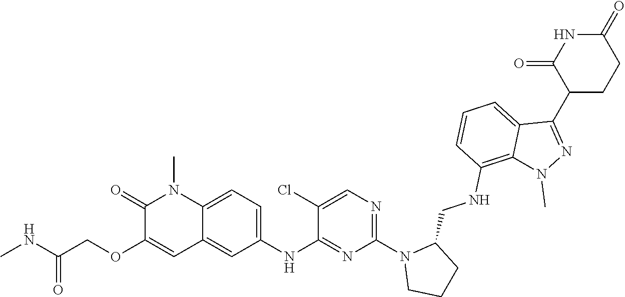 [SMILES: CNC(=O)COc1cc2cc(Nc3nc(N4CCC[C@H]4CNc4cccc5c(C6CCC(=O)NC6=O)nn(C)c45)ncc3Cl)ccc2n(C)c1=O] |
| 301 | 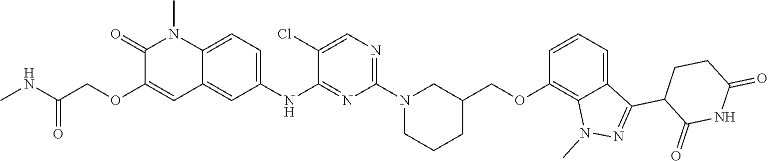 [SMILES: CNC(=O)COc1cc2cc(Nc3nc(N4CCCC(COc5cccc6c(C7CCC(=O)NC7=O)nn(C)c56)C4)ncc3Cl)ccc2n(C)c1=O] |
| 301a | 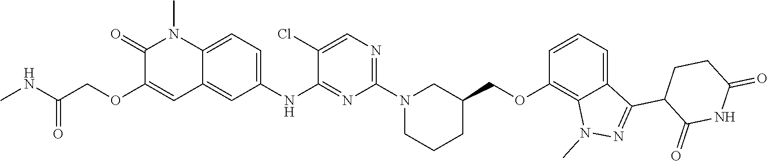 [SMILES: CNC(=O)COc1cc2cc(Nc3nc(N4CCC[C@H](COc5cccc6c(C7CCC(=O)NC7=O)nn(C)c56)C4)ncc3Cl)ccc2n(C)c1=O] |
| 301b | 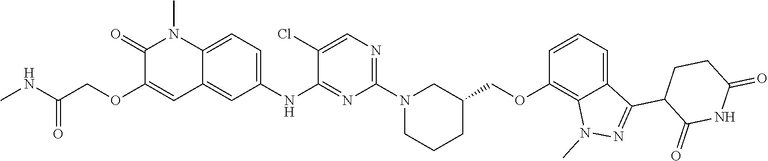 [SMILES: CNC(=O)COc1cc2cc(Nc3nc(N4CCC[C@@H](COc5cccc6c(C7CCC(=O)NC7=O)nn(C)c56)C4)ncc3Cl)ccc2n(C)c1=O] |
| 302 | 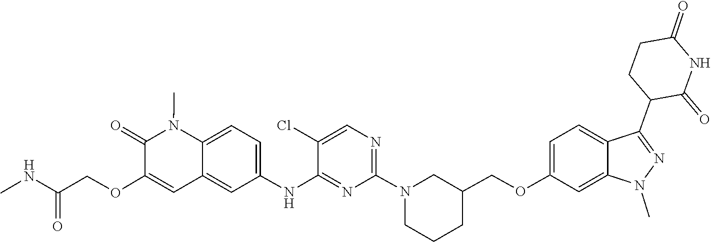 [SMILES: CNC(=O)COc1cc2cc(Nc3nc(N4CCCC(COc5ccc6c(C7CCC(=O)NC7=O)nn(C)c6c5)C4)ncc3Cl)ccc2n(C)c1=O] |
| 302a | 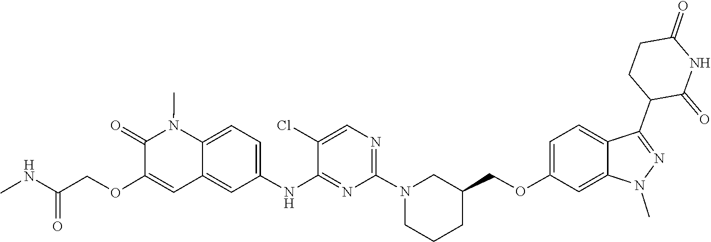 [SMILES: CNC(=O)COc1cc2cc(Nc3nc(N4CCC[C@H](COc5ccc6c(C7CCC(=O)NC7=O)nn(C)c6c5)C4)ncc3Cl)ccc2n(C)c1=O] |
| 302b | 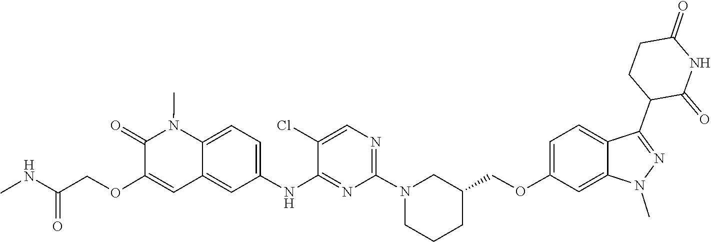 [SMILES: CNC(=O)COc1cc2cc(Nc3nc(N4CCC[C@@H](COc5ccc6c(C7CCC(=O)NC7=O)nn(C)c6c5)C4)ncc3Cl)ccc2n(C)c1=O] |
| 303 | 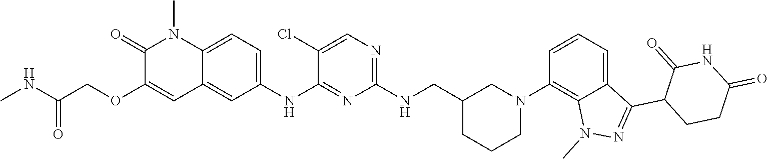 [SMILES: CNC(=O)COc1cc2cc(Nc3nc(NCC4CCCN(c5cccc6c(C7CCC(=O)NC7=O)nn(C)c56)C4)ncc3Cl)ccc2n(C)c1=O] |
| 303a | 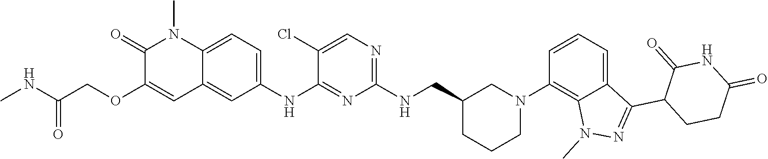 [SMILES: CNC(=O)COc1cc2cc(Nc3nc(NC[C@@H]4CCCN(c5cccc6c(C7CCC(=O)NC7=O)nn(C)c56)C4)ncc3Cl)ccc2n(C)c1=O] |
| 303b | 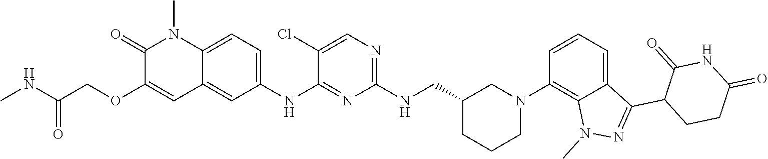 [SMILES: CNC(=O)COc1cc2cc(Nc3nc(NC[C@H]4CCCN(c5cccc6c(C7CCC(=O)NC7=O)nn(C)c56)C4)ncc3Cl)ccc2n(C)c1=O] |
| 304 | 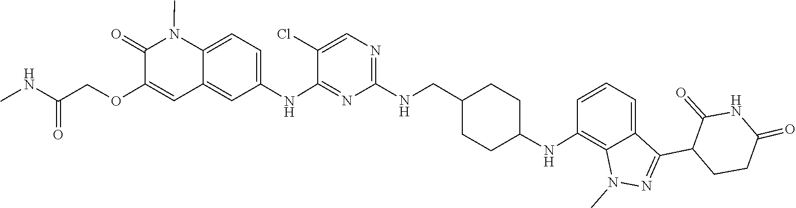 [SMILES: CNC(=O)COc1cc2cc(Nc3nc(NCC4CCC(Nc5cccc6c(C7CCC(=O)NC7=O)nn(C)c56)CC4)ncc3Cl)ccc2n(C)c1=O] |
| 304a | 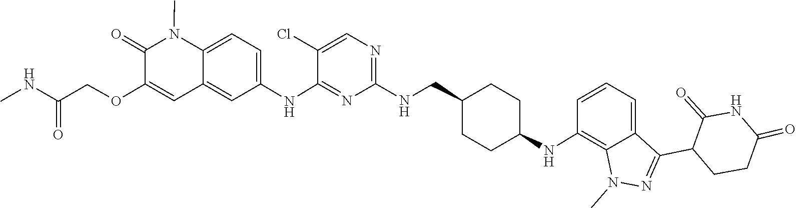 [SMILES: CNC(=O)COc1cc2cc(Nc3nc(NC[C@H]4CC[C@@H](Nc5cccc6c(C7CCC(=O)NC7=O)nn(C)c56)CC4)ncc3Cl)ccc2n(C)c1=O] |
| 304b | 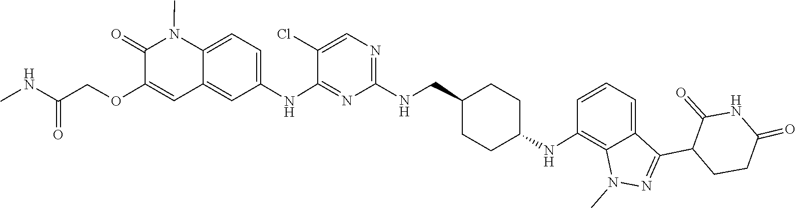 [SMILES: CNC(=O)COc1cc2cc(Nc3nc(NC[C@H]4CC[C@H](Nc5cccc6c(C7CCC(=O)NC7=O)nn(C)c56)CC4)ncc3Cl)ccc2n(C)c1=O] |
| 305 | 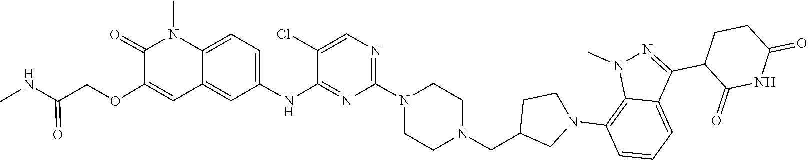 [SMILES: CNC(=O)COc1cc2cc(Nc3nc(N4CCN(CC5CCN(c6cccc7c(C8CCC(=O)NC8=O)nn(C)c67)C5)CC4)ncc3Cl)ccc2n(C)c1=O] |
| 305a | 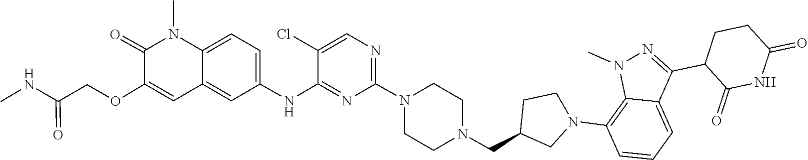 [SMILES: CNC(=O)COc1cc2cc(Nc3nc(N4CCN(C[C@H]5CCN(c6cccc7c(C8CCC(=O)NC8=O)nn(C)c67)C5)CC4)ncc3Cl)ccc2n(C)c1=O] |
| 305b | 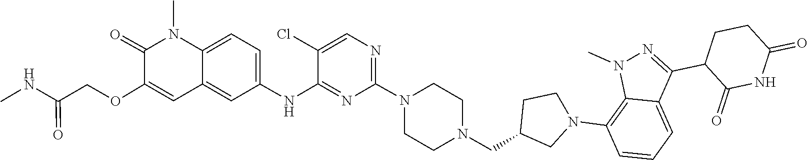 [SMILES: CNC(=O)COc1cc2cc(Nc3nc(N4CCN(C[C@@H]5CCN(c6cccc7c(C8CCC(=O)NC8=O)nn(C)c67)C5)CC4)ncc3Cl)ccc2n(C)c1=O] |
| 306 | 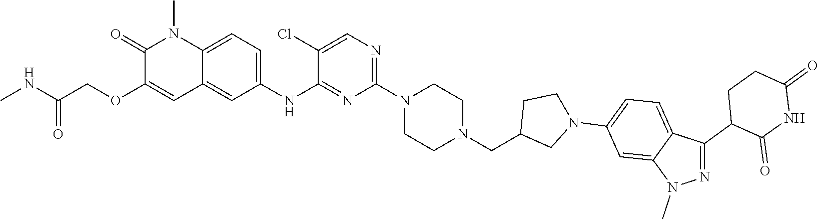 [SMILES: CNC(=O)COc1cc2cc(Nc3nc(N4CCN(CC5CCN(c6ccc7c(C8CCC(=O)NC8=O)nn(C)c7c6)C5)CC4)ncc3Cl)ccc2n(C)c1=O] |
| 306a | 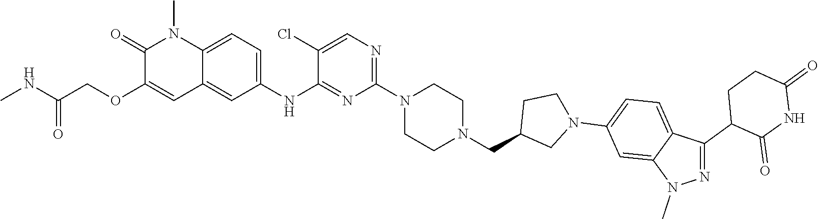 [SMILES: CNC(=O)COc1cc2cc(Nc3nc(N4CCN(C[C@H]5CCN(c6ccc7c(C8CCC(=O)NC8=O)nn(C)c7c6)C5)CC4)ncc3Cl)ccc2n(C)c1=O] |
| 306b | 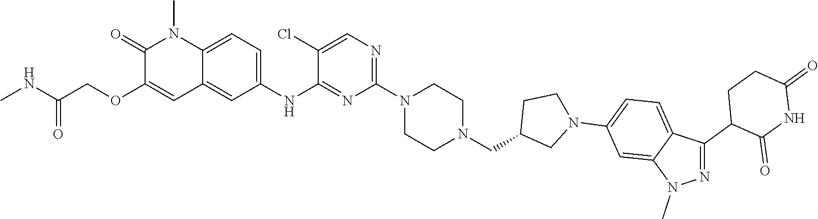 [SMILES: CNC(=O)COc1cc2cc(Nc3nc(N4CCN(C[C@@H]5CCN(c6ccc7c(C8CCC(=O)NC8=O)nn(C)c7c6)C5)CC4)ncc3Cl)ccc2n(C)c1=O] |
| 307 | 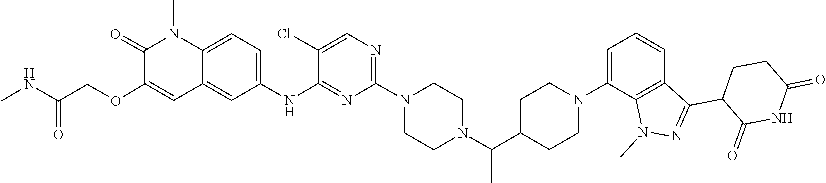 [SMILES: CNC(=O)COc1cc2cc(Nc3nc(N4CCN(C(C)C5CCN(c6cccc7c(C8CCC(=O)NC8=O)nn(C)c67)CC5)CC4)ncc3Cl)ccc2n(C)c1=O] |
| 307a | 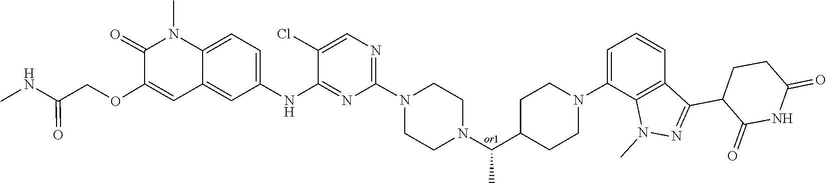 [SMILES: CNC(=O)COc1cc2cc(Nc3nc(N4CCN([C@@H](C)C5CCN(c6cccc7c(C8CCC(=O)NC8=O)nn(C)c67)CC5)CC4)ncc3Cl)ccc2n(C)c1=O] |
| 307b | 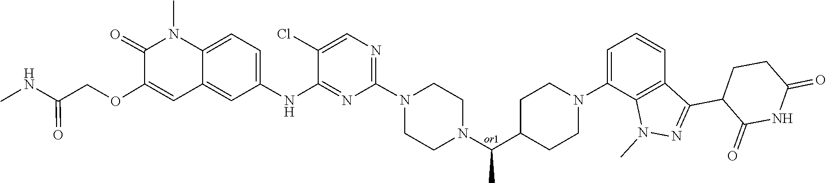 [SMILES: CNC(=O)COc1cc2cc(Nc3nc(N4CCN([C@H](C)C5CCN(c6cccc7c(C8CCC(=O)NC8=O)nn(C)c67)CC5)CC4)ncc3Cl)ccc2n(C)c1=O] |
| 308 | 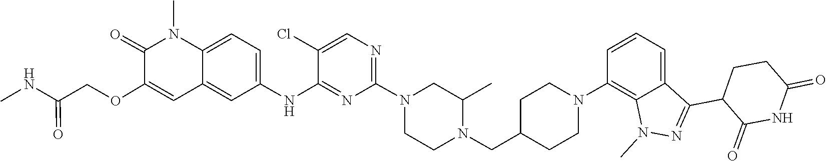 [SMILES: CNC(=O)COc1cc2cc(Nc3nc(N4CCN(CC5CCN(c6cccc7c(C8CCC(=O)NC8=O)nn(C)c67)CC5)C(C)C4)ncc3Cl)ccc2n(C)c1=O] |
| 308a | 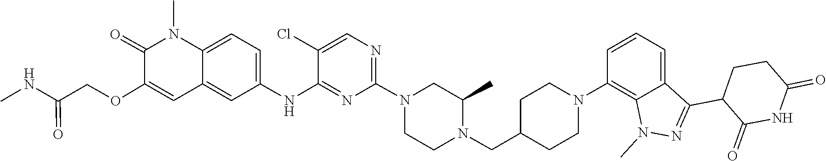 [SMILES: CNC(=O)COc1cc2cc(Nc3nc(N4CCN(CC5CCN(c6cccc7c(C8CCC(=O)NC8=O)nn(C)c67)CC5)[C@H](C)C4)ncc3Cl)ccc2n(C)c1=O] |
| 308b | 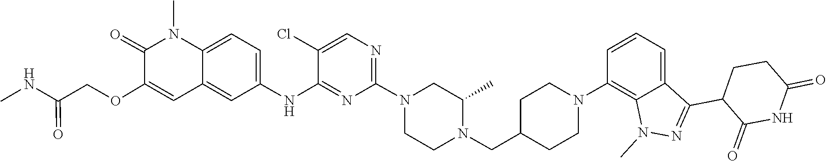 [SMILES: CNC(=O)COc1cc2cc(Nc3nc(N4CCN(CC5CCN(c6cccc7c(C8CCC(=O)NC8=O)nn(C)c67)CC5)[C@@H](C)C4)ncc3Cl)ccc2n(C)c1=O] |
| 309 | 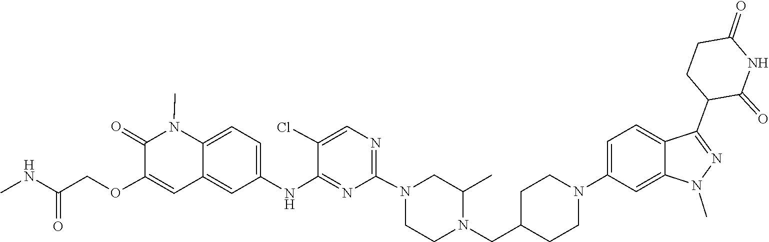 [SMILES: CNC(=O)COc1cc2cc(Nc3nc(N4CCN(CC5CCN(c6ccc7c(C8CCC(=O)NC8=O)nn(C)c7c6)CC5)C(C)C4)ncc3Cl)ccc2n(C)c1=O] |
| 309a | 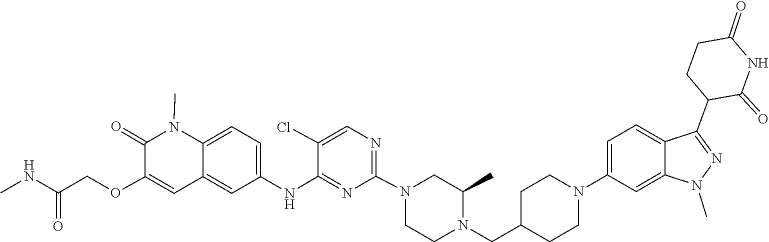 [SMILES: CNC(=O)COc1cc2cc(Nc3nc(N4CCN(CC5CCN(c6ccc7c(C8CCC(=O)NC8=O)nn(C)c7c6)CC5)[C@H](C)C4)ncc3Cl)ccc2n(C)c1=O] |
| 309b | 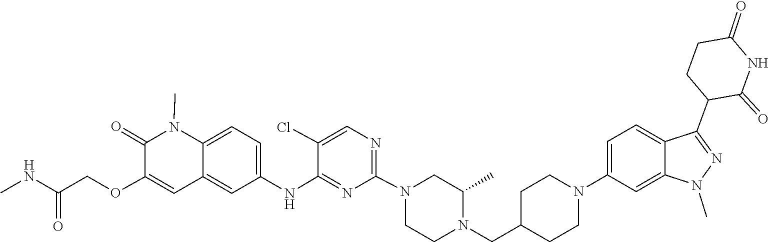 [SMILES: CNC(=O)COc1cc2cc(Nc3nc(N4CCN(CC5CCN(c6ccc7c(C8CCC(=O)NC8=O)nn(C)c7c6)CC5)[C@@H](C)C4)ncc3Cl)ccc2n(C)c1=O] |
| 310 | 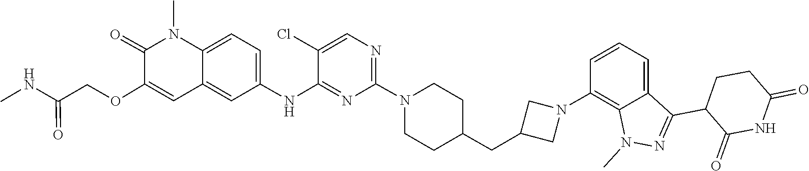 [SMILES: CNC(=O)COc1cc2cc(Nc3nc(N4CCC(CC5CN(c6cccc7c(C8CCC(=O)NC8=O)nn(C)c67)C5)CC4)ncc3Cl)ccc2n(C)c1=O] |
| 311 | 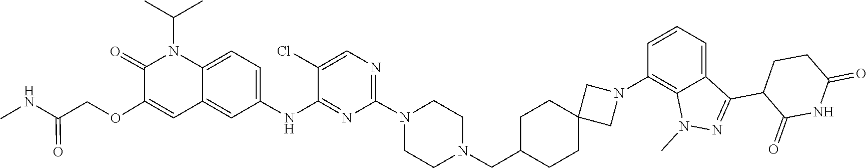 [SMILES: CNC(=O)COc1cc2cc(Nc3nc(N4CCN(CC5CCC6(CC5)CN(c5cccc7c(C8CCC(=O)NC8=O)nn(C)c57)C6)CC4)ncc3Cl)ccc2n(C(C)C)c1=O] |
| 312 | 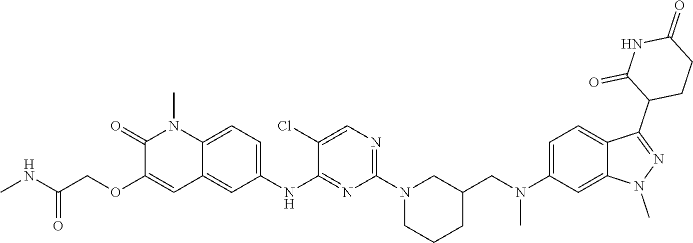 [SMILES: CNC(=O)COc1cc2cc(Nc3nc(N4CCCC(CN(C)c5ccc6c(C7CCC(=O)NC7=O)nn(C)c6c5)C4)ncc3Cl)ccc2n(C)c1=O] |
| 312a | 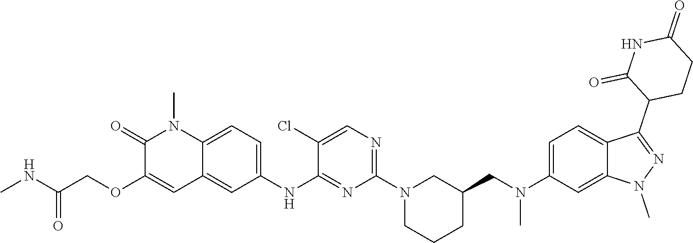 [SMILES: CNC(=O)COc1cc2cc(Nc3nc(N4CCC[C@H](CN(C)c5ccc6c(C7CCC(=O)NC7=O)nn(C)c6c5)C4)ncc3Cl)ccc2n(C)c1=O] |
| 313 | 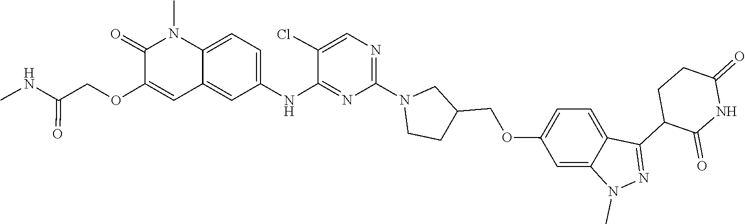 [SMILES: CNC(=O)COc1cc2cc(Nc3nc(N4CCC(COc5ccc6c(C7CCC(=O)NC7=O)nn(C)c6c5)C4)ncc3Cl)ccc2n(C)c1=O] |
| 313a | 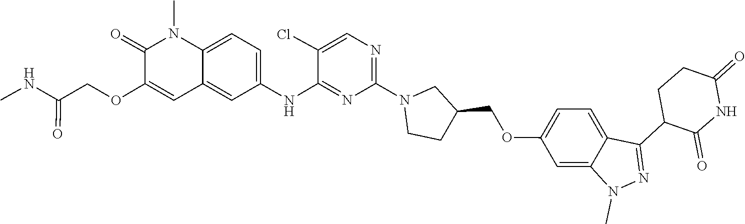 [SMILES: CNC(=O)COc1cc2cc(Nc3nc(N4CC[C@H](COc5ccc6c(C7CCC(=O)NC7=O)nn(C)c6c5)C4)ncc3Cl)ccc2n(C)c1=O] |
| 314 | 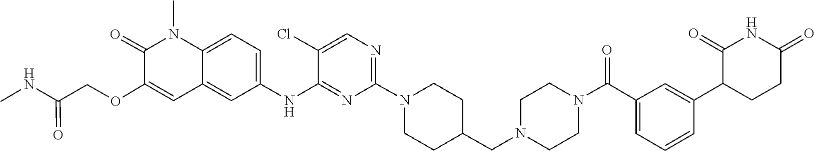 [SMILES: CNC(=O)COc1cc2cc(Nc3nc(N4CCC(CN5CCN(C(=O)c6cccc(C7CCC(=O)NC7=O)c6)CC5)CC4)ncc3Cl)ccc2n(C)c1=O] |
| 315 | 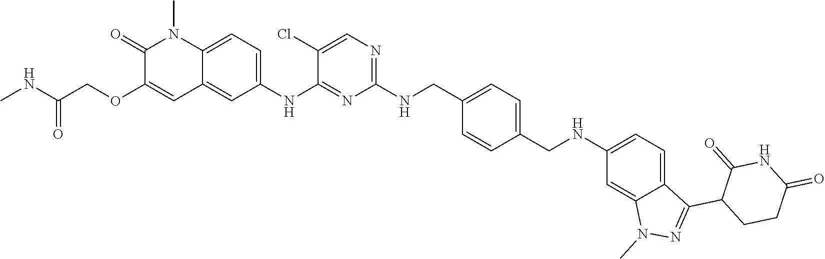 [SMILES: CNC(=O)COc1cc2cc(Nc3nc(NCc4ccc(CNc5ccc6c(C7CCC(=O)NC7=O)nn(C)c6c5)cc4)ncc3Cl)ccc2n(C)c1=O] |
| 316 | 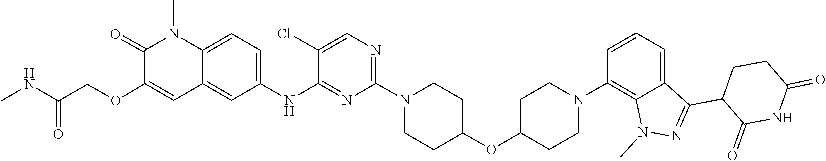 [SMILES: CNC(=O)COc1cc2cc(Nc3nc(N4CCC(OC5CCN(c6cccc7c(C8CCC(=O)NC8=O)nn(C)c67)CC5)CC4)ncc3Cl)ccc2n(C)c1=O] |
| 317 | 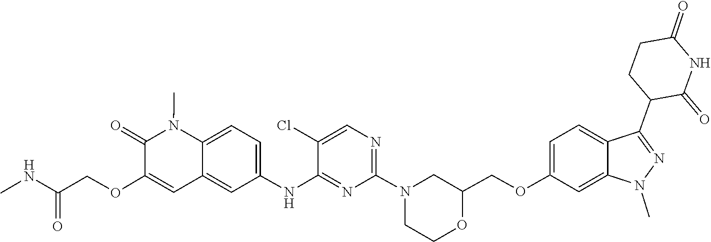 [SMILES: CNC(=O)COc1cc2cc(Nc3nc(N4CCOC(COc5ccc6c(C7CCC(=O)NC7=O)nn(C)c6c5)C4)ncc3Cl)ccc2n(C)c1=O] |
| 317a | 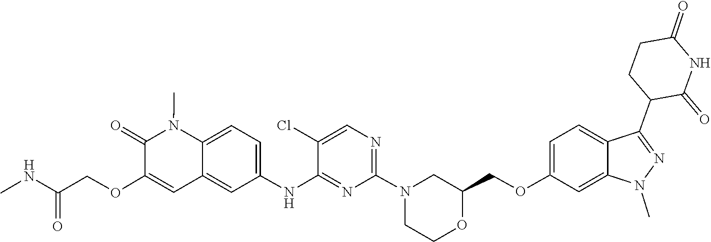 [SMILES: CNC(=O)COc1cc2cc(Nc3nc(N4CCO[C@H](COc5ccc6c(C7CCC(=O)NC7=O)nn(C)c6c5)C4)ncc3Cl)ccc2n(C)c1=O] |
| 318 | 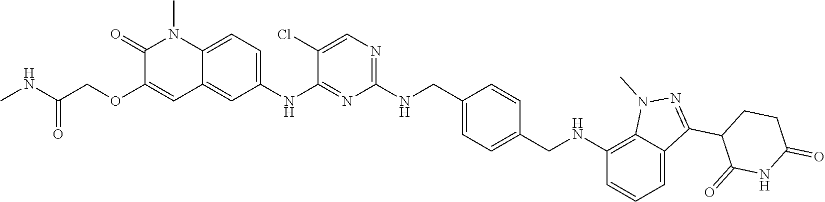 [SMILES: CNC(=O)COc1cc2cc(Nc3nc(NCc4ccc(CNc5cccc6c(C7CCC(=O)NC7=O)nn(C)c56)cc4)ncc3Cl)ccc2n(C)c1=O] |
| 319 | 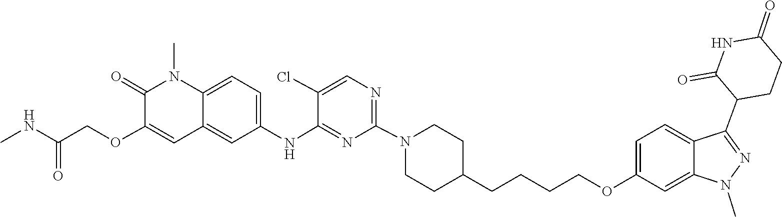 [SMILES: CNC(=O)COc1cc2cc(Nc3nc(N4CCC(CCCCOc5ccc6c(C7CCC(=O)NC7=O)nn(C)c6c5)CC4)ncc3Cl)ccc2n(C)c1=O] |
| 320 | 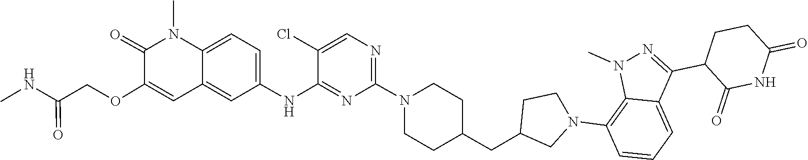 [SMILES: CNC(=O)COc1cc2cc(Nc3nc(N4CCC(CC5CCN(c6cccc7c(C8CCC(=O)NC8=O)nn(C)c67)C5)CC4)ncc3Cl)ccc2n(C)c1=O] |
| 321 | 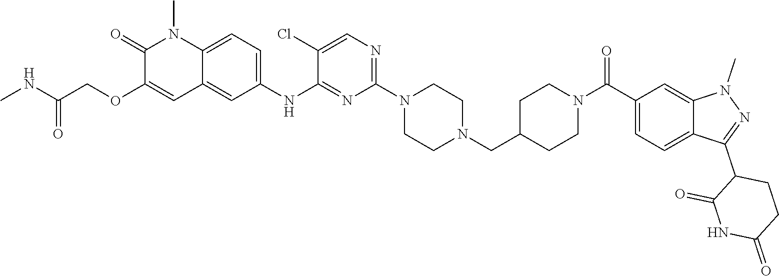 [SMILES: CNC(=O)COc1cc2cc(Nc3nc(N4CCN(CC5CCN(C(=O)c6ccc7c(C8CCC(=O)NC8=O)nn(C)c7c6)CC5)CC4)ncc3Cl)ccc2n(C)c1=O] |
| 322 | 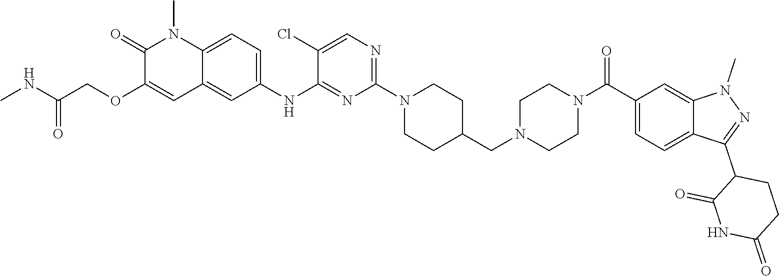 [SMILES: CNC(=O)COc1cc2cc(Nc3nc(N4CCC(CN5CCN(C(=O)c6ccc7c(C8CCC(=O)NC8=O)nn(C)c7c6)CC5)CC4)ncc3Cl)ccc2n(C)c1=O] |
| 323 | 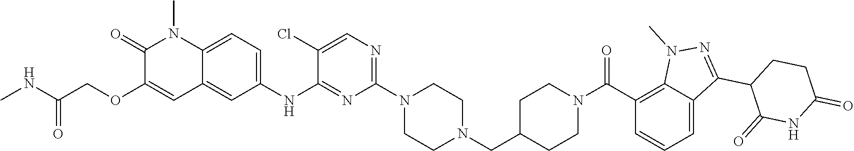 [SMILES: CNC(=O)COc1cc2cc(Nc3nc(N4CCN(CC5CCN(C(=O)c6cccc7c(C8CCC(=O)NC8=O)nn(C)c67)CC5)CC4)ncc3Cl)ccc2n(C)c1=O] |
| 324 | 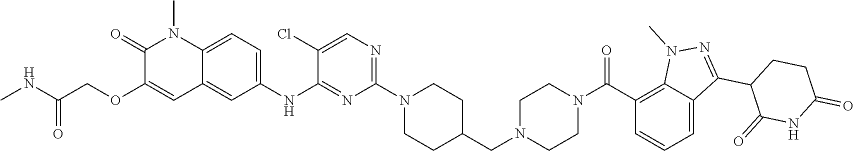 [SMILES: CNC(=O)COc1cc2cc(Nc3nc(N4CCC(CN5CCN(C(=O)c6cccc7c(C8CCC(=O)NC8=O)nn(C)c67)CC5)CC4)ncc3Cl)ccc2n(C)c1=O] |
| 325 | 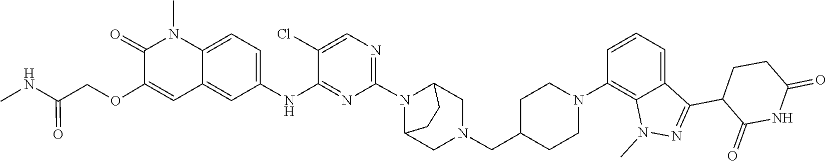 [SMILES: CNC(=O)COc1cc2cc(Nc3nc(N4C5CCC4CN(CC4CCN(c6cccc7c(C8CCC(=O)NC8=O)nn(C)c67)CC4)C5)ncc3Cl)ccc2n(C)c1=O] |
| 326 | 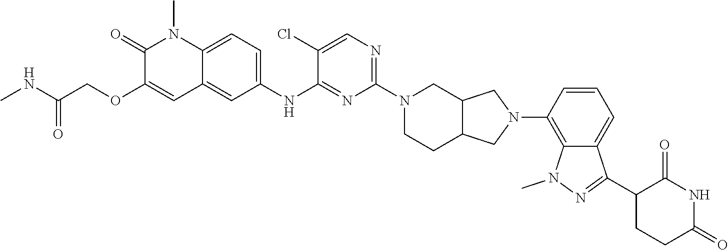 [SMILES: CNC(=O)COc1cc2cc(Nc3nc(N4CCC5CN(c6cccc7c(C8CCC(=O)NC8=O)nn(C)c67)CC5C4)ncc3Cl)ccc2n(C)c1=O] |
| 327 | 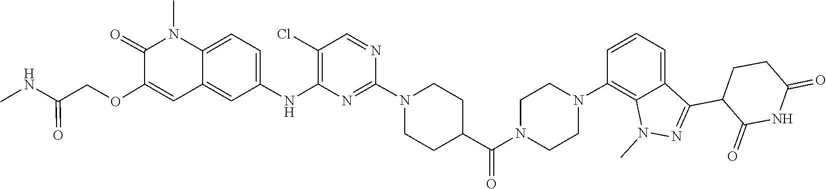 [SMILES: CNC(=O)COc1cc2cc(Nc3nc(N4CCC(C(=O)N5CCN(c6cccc7c(C8CCC(=O)NC8=O)nn(C)c67)CC5)CC4)ncc3Cl)ccc2n(C)c1=O] |
| 328 | 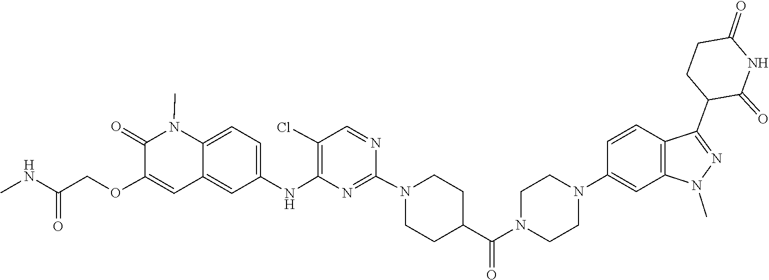 [SMILES: CNC(=O)COc1cc2cc(Nc3nc(N4CCC(C(=O)N5CCN(c6ccc7c(C8CCC(=O)NC8=O)nn(C)c7c6)CC5)CC4)ncc3Cl)ccc2n(C)c1=O] |
| 329 | 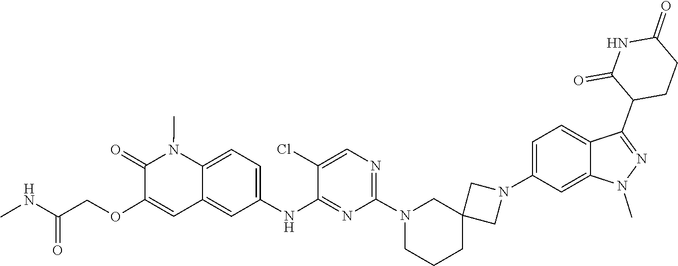 [SMILES: CNC(=O)COc1cc2cc(Nc3nc(N4CCCC5(CN(c6ccc7c(C8CCC(=O)NC8=O)nn(C)c7c6)C5)C4)ncc3Cl)ccc2n(C)c1=O] |
| 330 | 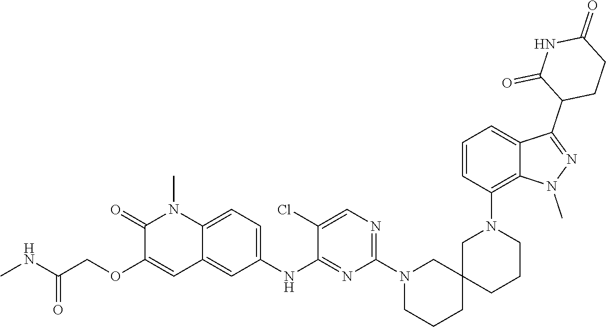 [SMILES: CNC(=O)COc1cc2cc(Nc3nc(N4CCCC5(CCCN(c6cccc7c(C8CCC(=O)NC8=O)nn(C)c67)C5)C4)ncc3Cl)ccc2n(C)c1=O] |
| 331 | 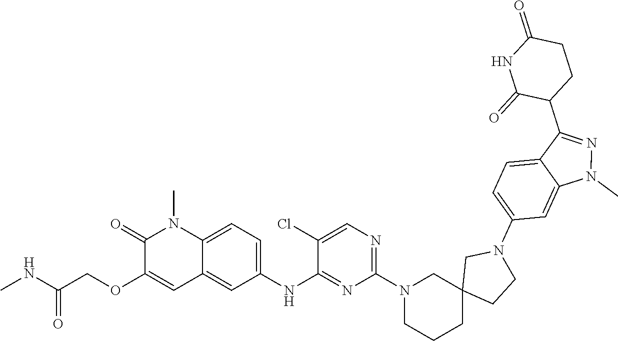 |
| 332 | 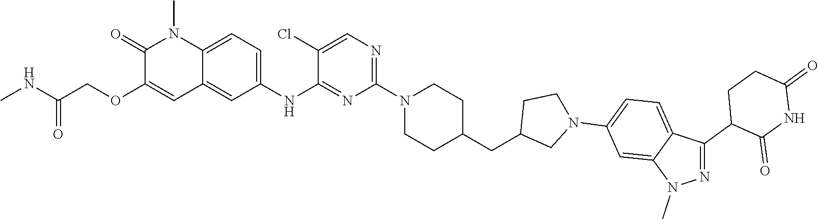 [SMILES: CNC(=O)COc1cc2cc(Nc3nc(N4CCC(CC5CCN(c6ccc7c(C8CCC(=O)NC8=O)nn(C)c7c6)C5)CC4)ncc3Cl)ccc2n(C)c1=O] |
| 333 | 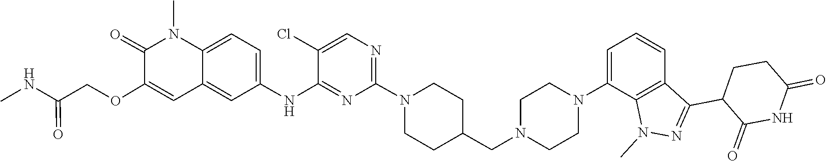 [SMILES: CNC(=O)COc1cc2cc(Nc3nc(N4CCC(CN5CCN(c6cccc7c(C8CCC(=O)NC8=O)nn(C)c67)CC5)CC4)ncc3Cl)ccc2n(C)c1=O] |
| 334 | 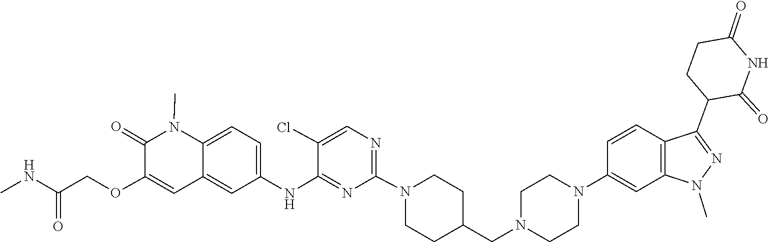 [SMILES: CNC(=O)COc1cc2cc(Nc3nc(N4CCC(CN5CCN(c6ccc7c(C8CCC(=O)NC8=O)nn(C)c7c6)CC5)CC4)ncc3Cl)ccc2n(C)c1=O] |
| 335 | 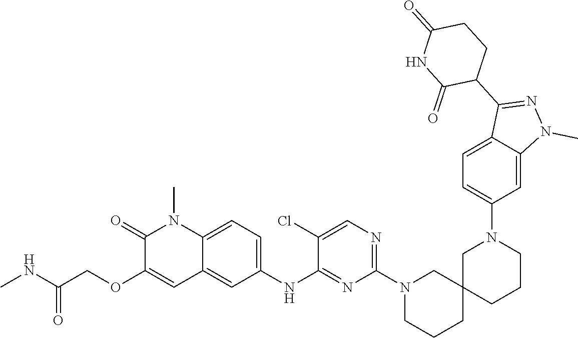 |
| 336 | 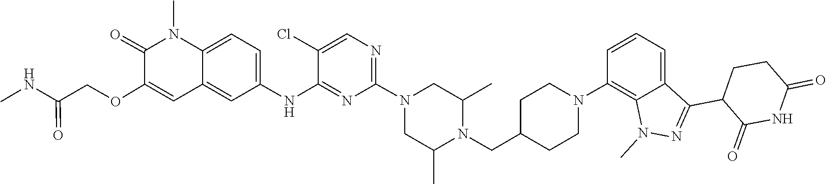 [SMILES: CNC(=O)COc1cc2cc(Nc3nc(N4CC(C)N(CC5CCN(c6cccc7c(C8CCC(=O)NC8=O)nn(C)c67)CC5)C(C)C4)ncc3Cl)ccc2n(C)c1=O] |
| 336a | 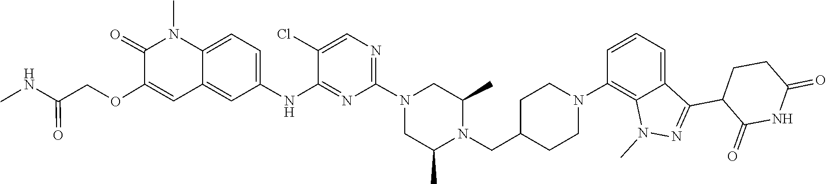 [SMILES: CNC(=O)COc1cc2cc(Nc3nc(N4C[C@@H](C)N(CC5CCN(c6cccc7c(C8CCC(=O)NC8=O)nn(C)c67)CC5)[C@@H](C)C4)ncc3Cl)ccc2n(C)c1=O] |
| 337 | 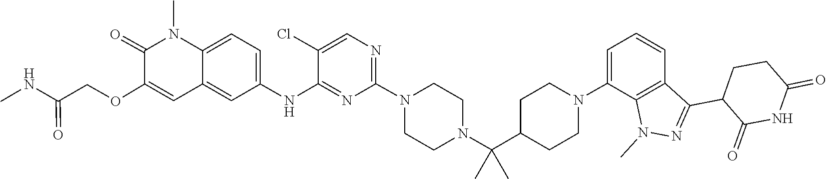 [SMILES: CNC(=O)COc1cc2cc(Nc3nc(N4CCN(C(C)(C)C5CCN(c6cccc7c(C8CCC(=O)NC8=O)nn(C)c67)CC5)CC4)ncc3Cl)ccc2n(C)c1=O] |
| 338 | 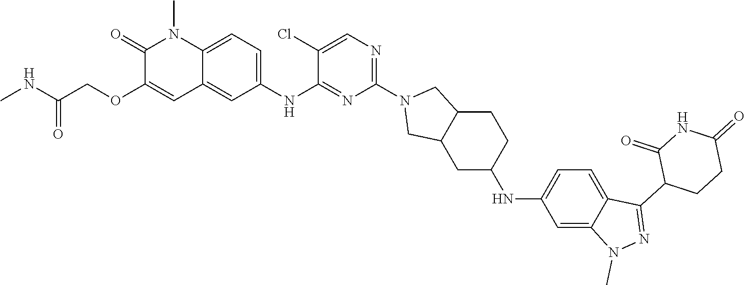 [SMILES: CNC(=O)COc1cc2cc(Nc3nc(N4CC5CCC(Nc6ccc7c(C8CCC(=O)NC8=O)nn(C)c7c6)CC5C4)ncc3Cl)ccc2n(C)c1=O] |
| 339 | 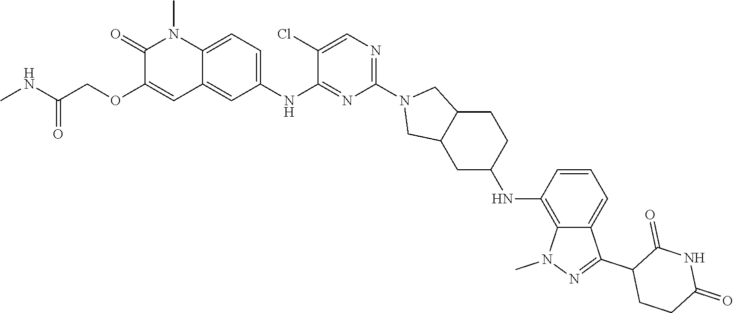 [SMILES: CNC(=O)COc1cc2cc(Nc3nc(N4CC5CCC(Nc6cccc7c(C8CCC(=O)NC8=O)nn(C)c67)CC5C4)ncc3Cl)ccc2n(C)c1=O] |
| 340 | 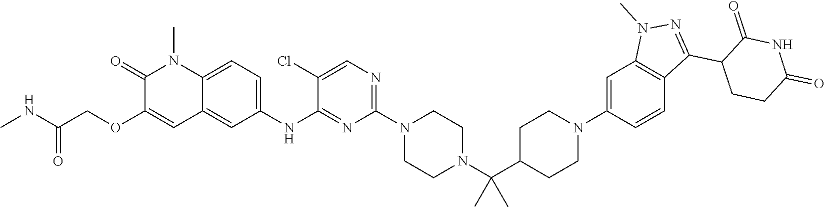 [SMILES: CNC(=O)COc1cc2cc(Nc3nc(N4CCN(C(C)(C)C5CCN(c6ccc7c(C8CCC(=O)NC8=O)nn(C)c7c6)CC5)CC4)ncc3Cl)ccc2n(C)c1=O] |
| 341 | 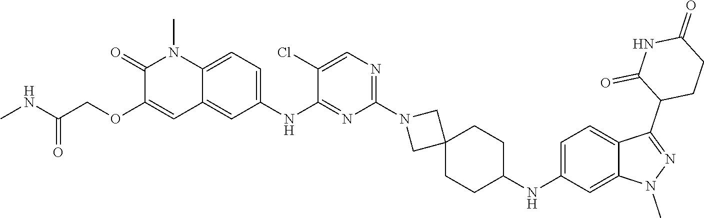 [SMILES: CNC(=O)COc1cc2cc(Nc3nc(N4CC5(CCC(Nc6ccc7c(C8CCC(=O)NC8=O)nn(C)c7c6)CC5)C4)ncc3Cl)ccc2n(C)c1=O] |
| 342 | 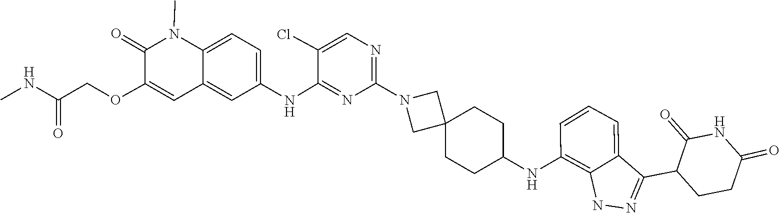 [SMILES: CNC(=O)COc1cc2cc(Nc3nc(N4CC5(CCC(Nc6cccc7c(C8CCC(=O)NC8=O)n[nH]c67)CC5)C4)ncc3Cl)ccc2n(C)c1=O] |
| 343 | 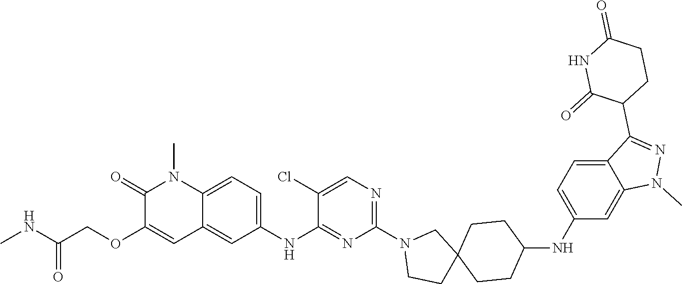 |
| 344 | 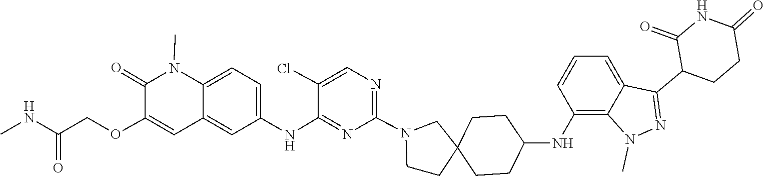 [SMILES: CNC(=O)COc1cc2cc(Nc3nc(N4CCC5(CCC(Nc6cccc7c(C8CCC(=O)NC8=O)nn(C)c67)CC5)C4)ncc3Cl)ccc2n(C)c1=O] |
| 345 | 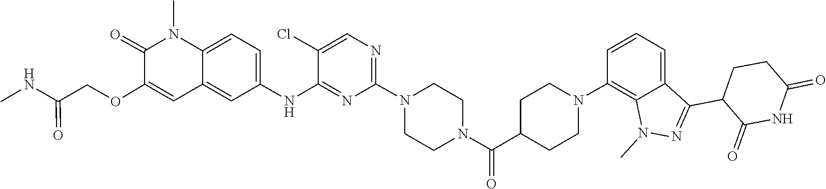 [SMILES: CNC(=O)COc1cc2cc(Nc3nc(N4CCN(C(=O)C5CCN(c6cccc7c(C8CCC(=O)NC8=O)nn(C)c67)CC5)CC4)ncc3Cl)ccc2n(C)c1=O] |
| 346 | 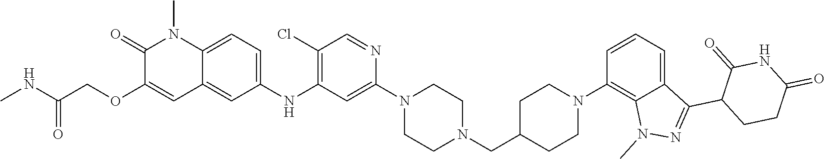 [SMILES: CNC(=O)COc1cc2cc(Nc3cc(N4CCN(CC5CCN(c6cccc7c(C8CCC(=O)NC8=O)nn(C)c67)CC5)CC4)ncc3Cl)ccc2n(C)c1=O] |
| 347 | 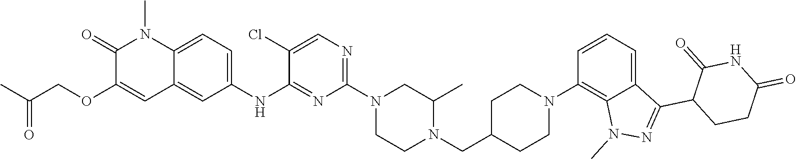 [SMILES: CC(=O)COc1cc2cc(Nc3nc(N4CCN(CC5CCN(c6cccc7c(C8CCC(=O)NC8=O)nn(C)c67)CC5)C(C)C4)ncc3Cl)ccc2n(C)c1=O] |
| 347a | 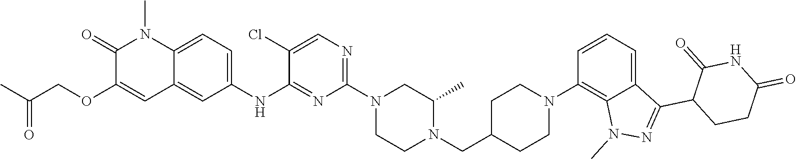 [SMILES: CC(=O)COc1cc2cc(Nc3nc(N4CCN(CC5CCN(c6cccc7c(C8CCC(=O)NC8=O)nn(C)c67)CC5)[C@@H](C)C4)ncc3Cl)ccc2n(C)c1=O] |
| 348 | 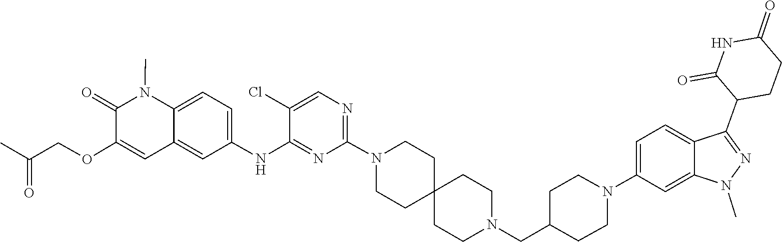 [SMILES: CC(=O)COc1cc2cc(Nc3nc(N4CCC5(CCN(CC6CCN(c7ccc8c(C9CCC(=O)NC9=O)nn(C)c8c7)CC6)CC5)CC4)ncc3Cl)ccc2n(C)c1=O] |

In certain compounds of Table C1, one or more stereogenic centers are denoted with the “V3000 enhanced stereochemical notation” (see: support.collaborativedrug.com/hc/en-us/articles/360020872171-Advanced-Stereochemistry-Registration-Atropisomers-Mixtures-Unknowns-and-Non-Tetrahedral-Chirality, accessed on Dec. 23, 2022 and Accelrys Chemical Representation Guide, Accelrys Software Inc., 2014, each of which is incorporated by reference herein in its entirety). Using this stereochemical notation, certain stereogenic centers are denoted with “abs”, “&x”, or “orx”, wherein x is an integer (e.g., 1 or 2). For avoidance of doubt, the stereochemical notations in Table C1 have the following meaning:
(1) When a stereogenic center (e.g., a stereogenic carbon) is depicted with “flat” bonds (i.e., none of the chemical bonds at the stereogenic center is depicted with wedges or dashes) in a structural formula, each of said stereogenic centers can independently adopt the (R)- or (S)-configurations. For example, the structure

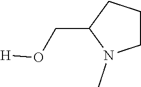

represents(S)-(1-methylpyrrolidin-2-yl)methanol, (R)-(1-methylpyrrolidin-2-yl)methanol, or a mixture thereof. As another non-limiting example, the structure

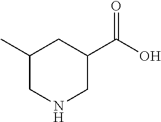

represents: (3S,5S)-5-methylpiperidine-3-carboxylic acid; (3R,5S)-5-methylpiperidine-3-carboxylic acid; (3S,5R)-5-methylpiperidine-3-carboxylic acid; (3R,5R)-5-methylpiperidine-3-carboxylic acid; or a mixture thereof.

When a stereogenic center or a plurality of stereogenic centers is depicted with wedges and dashes, the following notations are used:
(2) When a stereogenic center is denoted with “abs” or when a stereogenic center is not denoted with an enhanced stereochemical notation (e.g., “abs”, “&x”, or “orx”), the stereogenic center has the absolute configuration as depicted by the structural formula. For example, both of the structures

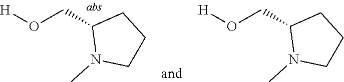

refer to(S)-(1-methylpyrrolidin-2-yl)methanol.
(3) When a stereogenic center is denoted with “orx” in a structural formula, the stereogenic center has been resolved but the configuration at the stereogenic center has not been determined. For example, the structure

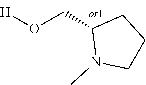

refers to one stereoisomer selected from the group consisting of(S)-(1-methylpyrrolidin-2-yl)methanol and (R)-(1-methylpyrrolidin-2-yl)methanol.
(4) When two or more stereogenic centers are denoted with “orx” in a structural formula, each of these stereogenic centers has been resolved but the configurations at said stereogenic centers have not been determined. Specifically:
   a. For any pair of stereogenic centers denoted with “orx” in a structural formula, when the numerical parts in the notation are different (e.g., two stereogenic centers denoted with “or1” and “or2” respectively), each stereogenic center is independently defined according to (3)(vide supra). For example, the structure

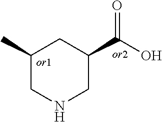

refers to one stereoisomer selected from the group consisting of:

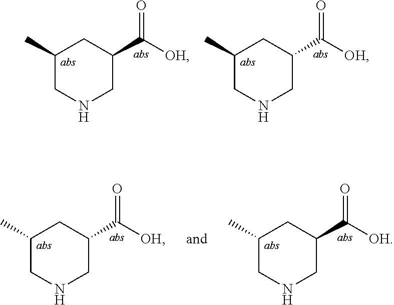

b. For any pair of stereogenic centers denoted with “orx” in a structural formula, when the numerical part in the notation is identical (e.g., two stereogenic centers each denoted with “or1”), the structural formula refers to one stereoisomer having the relative stereochemistry at these stereogenic centers as depicted in the structural formula, but the absolute configurations of these stereogenic centers have not been determined. For example, the structure

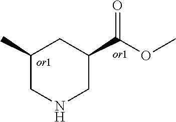

refers to one of the two “syn” stereoisomers:

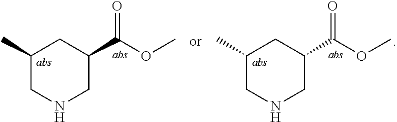

As another example, the structure

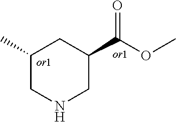

refers to one of the “anti” stereoisomers:

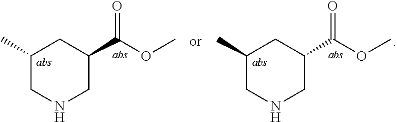

(5) When two or more stereogenic centers are denoted with “&x” in a structural formula, the structural formula refers to a mixture of stereoisomers that differ in the configuration at said stereogenic centers. Specifically:
   a. For any pair of stereogenic centers denoted with “&x” in a structural formula, when the numerical parts in the notation are different (e.g., two stereogenic centers denoted with “&1” and “&2” respectively), the structural formula refers to a mixture of stereoisomers at these two stereogenic centers, wherein the configuration at each stereogenic center can vary independently of one another. For example, the structure

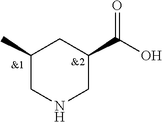

refers to a mixture of four stereoisomers:

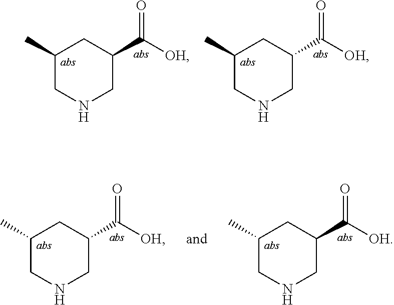

b. For any pair of stereogenic centers denoted with “&x” in a structural formula, when the numerical part in the notation is identical (e.g., two stereogenic centers each denoted with “&1”), the structural formula refers to a mixture of stereoisomers at these two or more stereogenic centers, wherein the relative configurations are as depicted in the structural formula. For example, the structure

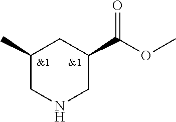

refers to a mixture of “syn” stereoisomers:

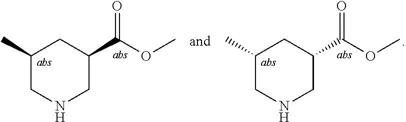

As another example, the structure

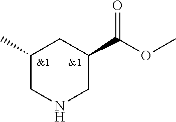

refers to a mixture of “anti” stereoisomers:

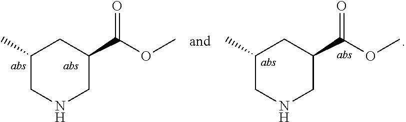

Exemplary compounds of Formula (I-aa) (e.g., Formula (I-a)) include compounds: 101, 102, 103, 104, 107, 108, 281, 287, 288, 298, 305, 305a, 305b, 306, 306a, 306b, 307, 307a, 307b, 308, 308a, 308b, 309, 309a, 309b, 310, 311, 316, 320, 321, 322, 323, 324, 325, 327, 328, 332, 333, 334, 336, 336a, 337, 340, 345, and 346, as depicted in Table C1, or pharmaceutically acceptable salts thereof.

Exemplary compounds of Formula (I-aa-1) (e.g., Formula (I-a-1)) include compounds: 101, 102, 107, 108, 281, 288, 305, 305a, 305b, 306, 306a, 306b, 307, 307a, 307b, 308, 308a, 308b, 309, 309a, 309b, 310, 320, 332, 333, 334, 336, 336a, 337, 340, and 346, as depicted in Table C1, or pharmaceutically acceptable salts thereof.

Exemplary compounds of Formula (I-aa-2) (e.g., Formula (I-a-2)) include compounds: 101, 102, 107, 108, 281, 288, 307, 307a, 307b, 308, 308a, 308b, 309, 309a, 309b, 333, 334, 336, 336a, 337, 340, and 346, as depicted in Table C1, or pharmaceutically acceptable salts thereof.

Exemplary compounds of Formula (I-aa-3) (e.g., Formula (I-a-3)) include compounds: 103, 104, 298, 311, and 325, as depicted in Table C1, or pharmaceutically acceptable salts thereof.

Exemplary compounds of Formula (I-aa-4) (e.g., Formula (I-a-4)) include compounds: 327, 328, and 345, as depicted in Table C1, or pharmaceutically acceptable salts thereof.

Exemplary compounds of Formula (I-bb) (e.g., Formula (I-b)) include compounds: 261, 261a, 261b, 266, 266a, 266b, 279, 279a, 279b, 280, 280a, 280b, 299, 299a, 300, 300a, 300b, 301, 301a, 301b, 302, 302a, 302b, 312, 312a, 313, 313a, 317, 317a, 338, 339, 341, 342, 343, and 344.

Exemplary compounds of Formula (I-bb-1) (e.g., Formula (I-b-1)) include compounds: 280, 280a, 280b, 338, 339, 341, 342, 343, and 344, as depicted in Table C1, or pharmaceutically acceptable salts thereof.

Exemplary compounds of Formula (I-bb-2) (e.g., Formula (I-b-2)) include compounds: 341 and 342, as depicted in Table C1, or pharmaceutically acceptable salts thereof.

In some embodiments, the compounds are selected from the group consisting of the compounds in Table C2, or pharmaceutically acceptable salts thereof.

TABLE C2

| Compound | |
|---|---|
| No. | Compound Name |
| 101 | 2-((6-((5-chloro-2-(4-((1-(3-(2,6-dioxopiperidin-3-yl)-1-methyl-1H- |
| | indazol-7-yl)piperidin-4-yl)methyl)piperazin-1-yl)pyrimidin-4- |
| | yl)amino)-1-methyl-2-oxo-1,2-dihydroquinolin-3-yl)oxy)-N- |
| | methylacetamide |
| 102 | 2-((6-((5-chloro-2-(4-((1-(3-(2,6-dioxopiperidin-3-yl)-1-methyl-1H- |
| | indazol-6-yl)piperidin-4-yl)methyl)piperazin-1-yl)pyrimidin-4- |
| | yl)amino)-1-methyl-2-oxo-1,2-dihydroquinolin-3-yl)oxy)-N- |
| | methylacetamide |
| 103 | 2-((6-((5-chloro-2-(4-((2-(3-(2,6-dioxopiperidin-3-yl)-1-methyl-1H- |
| | indazol-6-yl)-2-azaspiro[3.5]nonan-7-yl)methyl)piperazin-1- |
| | yl)pyrimidin-4-yl)amino)-1-methyl-2-oxo-1,2-dihydroquinolin-3- |
| | yl)oxy)-N-methylacetamide |
| 104 | 2-((6-((5-chloro-2-(4-((2-(3-(2,6-dioxopiperidin-3-yl)-1-methyl-1H- |
| | indazol-7-yl)-2-azaspiro[3.5]nonan-7-yl)methyl)piperazin-1- |
| | yl)pyrimidin-4-yl)amino)-1-methyl-2-oxo-1,2-dihydroquinolin-3- |
| | yl)oxy)-N-methylacetamide |
| 105 | N-((1-(5-chloro-4-((1-methyl-3-(2-(methylamino)-2-oxoethoxy)-2-oxo- |
| | 1,2-dihydroquinolin-6-yl)amino)pyrimidin-2-yl)piperidin-4-yl)methyl)- |
| | 3-(2,6-dioxopiperidin-3-yl)benzamide |
| 106 | 2-[[6-[[5-chloro-2-[3-[[4-(2,6-dioxo-3-piperidyl)anilino]methyl]-1- |
| | piperidyl]pyrimidin-4-yl]amino]-1-methyl-2-oxo-3-quinolyl]oxy]-N- |
| | methyl-acetamide |
| 106a | 2-((6-((5-chloro-2-((3R)-3-(((4-(2,6-dioxopiperidin-3- |
| | yl)phenyl)amino)methyl)piperidin-1-yl)pyrimidin-4-yl)amino)-1- |
| | methyl-2-oxo-1,2-dihydroquinolin-3-yl)oxy)-N-methylacetamide |
| 106b | 2-((6-((5-chloro-2-((3S)-3-(((4-(2,6-dioxopiperidin-3- |
| | yl)phenyl)amino)methyl)piperidin-1-yl)pyrimidin-4-yl)amino)-1- |
| | methyl-2-oxo-1,2-dihydroquinolin-3-yl)oxy)-N-methylacetamide |
| 107 | 2-((6-((5-chloro-2-(4-((1-(3-(2,6-dioxopiperidin-3-yl)-1-methyl-1H- |
| | indazol-7-yl)piperidin-4-yl)methyl)piperazin-1-yl)pyrimidin-4- |
| | yl)amino)-1-isopropyl-2-oxo-1,2-dihydroquinolin-3-yl)oxy)-N- |
| | methylacetamide |
| 108 | 2-((6-((5-chloro-2-(4-((1-(3-(2,6-dioxopiperidin-3-yl)-1-methyl-1H- |
| | indazol-6-yl)piperidin-4-yl)methyl)piperazin-1-yl)pyrimidin-4- |
| | yl)amino)-1-isopropyl-2-oxo-1,2-dihydroquinolin-3-yl)oxy)-N- |
| | methylacetamide |
| 260 | 2-[[6-[[5-chloro-2-[2-[[3-(2,6-dioxo-3- |
| | piperidyl)anilino]methyl]pyrrolidin-1-yl]pyrimidin-4-yl]amino]-1- |
| | methyl-2-oxo-3-quinolyl]oxy]-N-methyl-acetamide |
| 260a | 2-((6-((5-chloro-2-((2R)-2-(((3-(2,6-dioxopiperidin-3- |
| | yl)phenyl)amino)methyl)pyrrolidin-1-yl)pyrimidin-4-yl)amino)-1- |
| | methyl-2-oxo-1,2-dihydroquinolin-3-yl)oxy)-N-methylacetamide |
| 260b | 2-((6-((5-chloro-2-((2S)-2-(((3-(2,6-dioxopiperidin-3- |
| | yl)phenyl)amino)methyl)pyrrolidin-1-yl)pyrimidin-4-yl)amino)-1- |
| | methyl-2-oxo-1,2-dihydroquinolin-3-yl)oxy)-N-methylacetamide |
| 261 | 2-[[6-[[5-chloro-2-[3-[[[3-(2,6-dioxo-3-piperidyl)-1-methyl-indazol-6- |
| | yl]amino]methyl]-1-piperidyl]pyrimidin-4-yl]amino]-1-methyl-2-oxo-3- |
| | quinolyl]oxy]-N-methyl-acetamide |
| 261a | 2-((6-((5-chloro-2-((3S)-3-(((3-(2,6-dioxopiperidin-3-yl)-1-methyl-1H- |
| | indazol-6-yl)amino)methyl)piperidin-1-yl)pyrimidin-4-yl)amino)-1- |
| | methyl-2-oxo-1,2-dihydroquinolin-3-yl)oxy)-N-methylacetamide |
| 261b | 2-((6-((5-chloro-2-((3R)-3-(((3-(2,6-dioxopiperidin-3-yl)-1-methyl-1H- |
| | indazol-6-yl)amino)methyl)piperidin-1-yl)pyrimidin-4-yl)amino)-1- |
| | methyl-2-oxo-1,2-dihydroquinolin-3-yl)oxy)-N-methylacetamide |
| 262 | 2-[[6-[[5-chloro-2-[[4-[3-(2,6-dioxo-3- |
| | piperidyl)anilino]cyclohexyl]methylamino]pyrimidin-4-yl]amino]-1- |
| | methyl-2-oxo-3-quinolyl]oxy]-N-methyl-acetamide |
| 262a | 2-((6-((5-chloro-2-((((1s,4s)-4-((3-(2,6-dioxopiperidin-3- |
| | yl)phenyl)amino)cyclohexyl)methyl)amino)pyrimidin-4-yl)amino)-1- |
| | methyl-2-oxo-1,2-dihydroquinolin-3-yl)oxy)-N-methylacetamide |
| 262b | 2-((6-((5-chloro-2-((((1r,4r)-4-((3-(2,6-dioxopiperidin-3- |
| | yl)phenyl)amino)cyclohexyl)methyl)amino)pyrimidin-4-yl)amino)-1- |
| | methyl-2-oxo-1,2-dihydroquinolin-3-yl)oxy)-N-methylacetamide |
| 263 | 2-((6-((5-chloro-2-(4-(2-((3-(2,6-dioxopiperidin-3- |
| | yl)phenyl)amino)ethyl)piperidin-1-yl)pyrimidin-4-yl)amino)-1-methyl- |
| | 2-oxo-1,2-dihydroquinolin-3-yl)oxy)-N-methylacetamide |
| 265 | 2-((6-((5-chloro-2-(4-(((4-(2,6-dioxopiperidin-3- |
| | yl)phenyl)amino)methyl)piperidin-1-yl)pyrimidin-4-yl)amino)-1- |
| | methyl-2-oxo-1,2-dihydroquinolin-3-yl)oxy)-N-methylacetamide |
| 266 | 2-[[6-[[5-chloro-2-[3-[[[3-(2,6-dioxo-3-piperidyl)-1-methyl-indazol-7- |
| | yl]amino]methyl]-1-piperidyl]pyrimidin-4-yl]amino]-1-methyl-2-oxo-3- |
| | quinolyl]oxy]-N-methyl-acetamide |
| 266a | 2-((6-((5-chloro-2-((3S)-3-(((3-(2,6-dioxopiperidin-3-yl)-1-methyl-1H- |
| | indazol-7-yl)amino)methyl)piperidin-1-yl)pyrimidin-4-yl)amino)-1- |
| | methyl-2-oxo-1,2-dihydroquinolin-3-yl)oxy)-N-methylacetamide |
| 266b | 2-((6-((5-chloro-2-((3R)-3-(((3-(2,6-dioxopiperidin-3-yl)-1-methyl-1H- |
| | indazol-7-yl)amino)methyl)piperidin-1-yl)pyrimidin-4-yl)amino)-1- |
| | methyl-2-oxo-1,2-dihydroquinolin-3-yl)oxy)-N-methylacetamide |
| 275 | 2-[[6-[[5-chloro-2-[3-[[3-(2,6-dioxo-3-piperidyl)anilino]methyl]-1- |
| | piperidyl]pyrimidin-4-yl]amino]-1-methyl-2-oxo-3-quinolyl]oxy]-N- |
| | methyl-acetamide |
| 275a | 2-((6-((5-chloro-2-((3R)-3-(((3-(2,6-dioxopiperidin-3- |
| | yl)phenyl)amino)methyl)piperidin-1-yl)pyrimidin-4-yl)amino)-1- |
| | methyl-2-oxo-1,2-dihydroquinolin-3-yl)oxy)-N-methylacetamide |
| 275b | 2-((6-((5-chloro-2-((3S)-3-(((3-(2,6-dioxopiperidin-3- |
| | yl)phenyl)amino)methyl)piperidin-1-yl)pyrimidin-4-yl)amino)-1- |
| | methyl-2-oxo-1,2-dihydroquinolin-3-yl)oxy)-N-methylacetamide |
| 277 | 2-[[6-[[5-chloro-2-[[4-[4-(2,6-dioxo-3- |
| | piperidyl)anilino]cyclohexyl]methylamino]pyrimidin-4-yl]amino]-1- |
| | methyl-2-oxo-3-quinolyl]oxy]-N-methyl-acetamide |
| 277a | 2-((6-((5-chloro-2-((((1r,4r)-4-((4-(2,6-dioxopiperidin-3- |
| | yl)phenyl)amino)cyclohexyl)methyl)amino)pyrimidin-4-yl)amino)-1- |
| | methyl-2-oxo-1,2-dihydroquinolin-3-yl)oxy)-N-methylacetamide |
| 277b | 2-((6-((5-chloro-2-((((1s,4s)-4-((4-(2,6-dioxopiperidin-3- |
| | yl)phenyl)amino) cyclohexyl)methyl)amino)pyrimidin-4-yl)amino)-1- |
| | methyl-2-oxo-1,2-dihydroquinolin-3-yl)oxy)-N-methylacetamide |
| 279 | 2-[[6-[[5-chloro-2-[2-[[[3-(2,6-dioxo-3-piperidyl)-1-methyl-indazol-6- |
| | yl]amino]methyl]pyrrolidin-1-yl]pyrimidin-4-yl]amino]-1-methyl-2- |
| | oxo-3-quinolyl]oxy]-N-methyl-acetamide |
| 279a | 2-((6-((5-chloro-2-((2S)-2-(((3-(2,6-dioxopiperidin-3-yl)-1-methyl-1H- |
| | indazol-6-yl)amino)methyl)pyrrolidin-1-yl)pyrimidin-4-yl)amino)-1- |
| | methyl-2-oxo-1,2-dihydroquinolin-3-yl)oxy)-N-methylacetamide |
| 279b | 2-((6-((5-chloro-2-((2R)-2-(((3-(2,6-dioxopiperidin-3-yl)-1-methyl-1H- |
| | indazol-6-yl)amino)methyl)pyrrolidin-1-yl)pyrimidin-4-yl)amino)-1- |
| | methyl-2-oxo-1,2-dihydroquinolin-3-yl)oxy)-N-methylacetamide |
| 280 | 2-[[6-[[5-chloro-2-[3-[[3-(2,6-dioxo-3-piperidyl)-1-methyl-indazol-6- |
| | yl]amino]-1-piperidyl]pyrimidin-4-yl]amino]-1-methyl-2-oxo-3- |
| | quinolyl]oxy]-N-methyl-acetamide |
| 280a | 2-((6-((5-chloro-2-((3S)-3-((3-(2,6-dioxopiperidin-3-yl)-1-methyl-1H- |
| | indazol-6-yl)amino)piperidin-1-yl)pyrimidin-4-yl)amino)-1-methyl-2- |
| | oxo-1,2-dihydroquinolin-3-yl)oxy)-N-methylacetamide |
| 280b | 2-((6-((5-chloro-2-((3R)-3-((3-(2,6-dioxopiperidin-3-yl)-1-methyl-1H- |
| | indazol-6-yl)amino)piperidin-1-yl)pyrimidin-4-yl)amino)-1-methyl-2- |
| | oxo-1,2-dihydroquinolin-3-yl)oxy)-N-methylacetamide |
| 281 | 2-((6-((5-chloro-2-(4-((1-(3-(2,6-dioxopiperidin-3-yl)-1-methyl-1H- |
| | indazol-7-yl)piperidin-4-yl)methyl)piperidin-1-yl)pyrimidin-4- |
| | yl)amino)-1-methyl-2-oxo-1,2-dihydroquinolin-3-yl)oxy)-N- |
| | methylacetamide |
| 282 | 2-((6-((5-chloro-2-(4-(1-(3-(2,6-dioxopiperidin-3-yl)-1-methyl-1H- |
| | indazol-6-yl)azetidin-3-yl)piperazin-1-yl)pyrimidin-4-yl)amino)-1- |
| | methyl-2-oxo-1,2-dihydroquinolin-3-yl)oxy)-N-methylacetamide |
| 283 | 2-((6-((5-chloro-2-(4-(1-(3-(2,6-dioxopiperidin-3-yl)-1-methyl-1H- |
| | indazol-7-yl)azetidin-3-yl)piperazin-1-yl)pyrimidin-4-yl)amino)-1- |
| | methyl-2-oxo-1,2-dihydroquinolin-3-yl)oxy)-N-methylacetamide |
| 284 | 2-((6-((5-chloro-2-(4-(2-((4-(2,6-dioxopiperidin-3- |
| | yl)phenyl)amino)ethyl)piperidin-1-yl)pyrimidin-4-yl)amino)-1-methyl- |
| | 2-oxo-1,2-dihydroquinolin-3-yl)oxy)-N-methylacetamide |
| 286 | 2-((6-((5-chloro-2-(4-(1-(3-(2,6-dioxopiperidin-3-yl)-1-methyl-1H- |
| | indazol-7-yl)azetidin-3-yl)piperidin-1-yl)pyrimidin-4-yl)amino)-1- |
| | methyl-2-oxo-1,2-dihydroquinolin-3-yl)oxy)-N-methylacetamide |
| 287 | 2-((6-((5-chloro-2-(4-((1-(3-(2,6-dioxopiperidin-3-yl)-1-methyl-1H- |
| | indazol-6-yl)piperidin-4-yl)oxy)piperidin-1-yl)pyrimidin-4-yl)amino)-1- |
| | methyl-2-oxo-1,2-dihydroquinolin-3-yl)oxy)-N-methylacetamide |
| 288 | 2-((6-((5-chloro-2-(4-((1-(3-(2,6-dioxopiperidin-3-yl)-1-methyl-1H- |
| | indazol-6-yl)piperidin-4-yl)methyl)piperidin-1-yl)pyrimidin-4- |
| | yl)amino)-1-methyl-2-oxo-1,2-dihydroquinolin-3-yl)oxy)-N- |
| | methylacetamide |
| 289 | 2-[[6-[[5-chloro-2-[[4-[[3-(2,6-dioxo-3-piperidyl)-1-methyl-indazol-6- |
| | yl]amino]cyclohexyl]methylamino]pyrimidin-4-yl]amino]-1-methyl-2- |
| | oxo-3-quinolyl]oxy]-N-methyl-acetamide |
| 289a | 2-((6-((5-chloro-2-((((1s,4s)-4-((3-(2,6-dioxopiperidin-3-yl)-1-methyl- |
| | 1H-indazol-6-yl)amino)cyclohexyl)methyl)amino)pyrimidin-4- |
| | yl)amino)-1-methyl-2-oxo-1,2-dihydroquinolin-3-yl)oxy)-N- |
| | methylacetamide |
| 289b | 2-((6-((5-chloro-2-((((1r,4r)-4-((3-(2,6-dioxopiperidin-3-yl)-1-methyl- |
| | 1H-indazol-6-yl)amino)cyclohexyl)methyl)amino)pyrimidin-4- |
| | yl)amino)-1-methyl-2-oxo-1,2-dihydroquinolin-3-yl)oxy)-N- |
| | methylacetamide |
| 290 | 2-[[6-[[5-chloro-2-[3-[2-[[3-(2,6-dioxo-3-piperidyl)-1-methyl-indazol- |
| | 6-yl]amino]ethyl]-1-piperidyl]pyrimidin-4-yl]amino]-1-methyl-2-oxo-3- |
| | quinolyl]oxy]-N-methyl-acetamide |
| 290a | rel-2-((6-((5-chloro-2-((3R)-3-(2-((3-(2,6-dioxopiperidin-3-yl)-1- |
| | methyl-1H-indazol-6-yl)amino)ethyl)piperidin-1-yl)pyrimidin-4- |
| | yl)amino)-1-methyl-2-oxo-1,2-dihydroquinolin-3-yl)oxy)-N- |
| | methylacetamide |
| 290b | rel-2-((6-((5-chloro-2-((3S)-3-(2-((3-(2,6-dioxopiperidin-3-yl)-1- |
| | methyl-1H-indazol-6-yl)amino)ethyl)piperidin-1-yl)pyrimidin-4- |
| | yl)amino)-1-methyl-2-oxo-1,2-dihydroquinolin-3-yl)oxy)-N- |
| | methylacetamide |
| 292 | 2-((6-((5-chloro-2-(4-(1-(3-(2,6-dioxopiperidin-3-yl)-1-methyl-1H- |
| | indazol-6-yl)azetidin-3-yl)piperidin-1-yl)pyrimidin-4-yl)amino)-1- |
| | methyl-2-oxo-1,2-dihydroquinolin-3-yl)oxy)-N-methylacetamide |
| 293 | 2-[[6-[[5-chloro-2-[[4-[[[3-(2,6-dioxo-3-piperidyl)-1-methyl-indazol-6- |
| | yl]amino]methyl]cyclohexyl]methylamino]pyrimidin-4-yl]amino]-1- |
| | methyl-2-oxo-3-quinolyl]oxy]-N-methyl-acetamide |
| 293a | 2-((6-((5-chloro-2-((((1s,4s)-4-(((3-(2,6-dioxopiperidin-3-yl)-1-methyl- |
| | 1H-indazol-6-yl)amino)methyl)cyclohexyl)methyl)amino)pyrimidin-4- |
| | yl)amino)-1-methyl-2-oxo-1,2-dihydroquinolin-3-yl)oxy)-N- |
| | methylacetamide |
| 293b | 2-((6-((5-chloro-2-((((1r,4r)-4-(((3-(2,6-dioxopiperidin-3-yl)-1-methyl- |
| | 1H-indazol-6-yl)amino)methyl)cyclohexyl)methyl)amino)pyrimidin-4- |
| | yl)amino)-1-methyl-2-oxo-1,2-dihydroquinolin-3-yl)oxy)-N- |
| | methylacetamide |
| 294 | 2-[[6-[[5-chloro-2-[3-[2-[[3-(2,6-dioxo-3-piperidyl)-1-methyl-indazol- |
| | 7-yl]amino]ethyl]-1-piperidyl]pyrimidin-4-yl]amino]-1-methyl-2-oxo-3- |
| | quinolyl]oxy]-N-methyl-acetamide |
| 294a | rel-2-((6-((5-chloro-2-((3S)-3-(2-((3-(2,6-dioxopiperidin-3-yl)-1- |
| | methyl-1H-indazol-7-yl)amino)ethyl)piperidin-1-yl)pyrimidin-4- |
| | yl)amino)-1-methyl-2-oxo-1,2-dihydroquinolin-3-yl)oxy)-N- |
| | methylacetamide |
| 294b | rel-2-((6-((5-chloro-2-((3R)-3-(2-((3-(2,6-dioxopiperidin-3-yl)-1- |
| | methyl-1H-indazol-7-yl)amino)ethyl)piperidin-1-yl)pyrimidin-4- |
| | yl)amino)-1-methyl-2-oxo-1,2-dihydroquinolin-3-yl)oxy)-N- |
| | methylacetamide |
| 295 | 2-[[6-[[5-chloro-2-[[1-[3-(2,6-dioxo-3-piperidyl)-1-methyl-indazol-6- |
| | yl]-3-piperidyl]methylamino]pyrimidin-4-yl]amino]-1-methyl-2-oxo-3- |
| | quinolyl]oxy]-N-methyl-acetamide |
| 295a | 2-((6-((5-chloro-2-((((3R)-1-(3-(2,6-dioxopiperidin-3-yl)-1-methyl-1H- |
| | indazol-6-yl)piperidin-3-yl)methyl)amino)pyrimidin-4-yl)amino)-1- |
| | methyl-2-oxo-1,2-dihydroquinolin-3-yl)oxy)-N-methylacetamide |
| 295b | 2-((6-((5-chloro-2-((((3S)-1-(3-(2,6-dioxopiperidin-3-yl)-1-methyl-1H- |
| | indazol-6-yl)piperidin-3-yl)methyl)amino)pyrimidin-4-yl)amino)-1- |
| | methyl-2-oxo-1,2-dihydroquinolin-3-yl)oxy)-N-methylacetamide |
| 296 | 2-((6-((5-chloro-2-(4-(3-((3-(2,6-dioxopiperidin-3-yl)-1-methyl-1H- |
| | indazol-6-yl)oxy)propyl)piperidin-1-yl)pyrimidin-4-yl)amino)-1- |
| | methyl-2-oxo-1,2-dihydroquinolin-3-yl)oxy)-N-methylacetamide |
| 297 | 2-[[6-[[5-chloro-2-[[4-[[[3-(2,6-dioxo-3-piperidyl)-1-methyl-indazol-7- |
| | yl]amino]methyl]cyclohexyl]methylamino]pyrimidin-4-yl]amino]-1- |
| | methyl-2-oxo-3-quinolyl]oxy]-N-methyl-acetamide |
| 297a | 2-((6-((5-chloro-2-((((1s,4s)-4-(((3-(2,6-dioxopiperidin-3-yl)-1-methyl- |
| | 1H-indazol-7-yl)amino)methyl)cyclohexyl)methyl)amino)pyrimidin-4- |
| | yl)amino)-1-methyl-2-oxo-1,2-dihydroquinolin-3-yl)oxy)-N- |
| | methylacetamide |
| 297b | 2-((6-((5-chloro-2-((((1r,4r)-4-(((3-(2,6-dioxopiperidin-3-yl)-1-methyl- |
| | 1H-indazol-7-yl)amino)methyl)cyclohexyl)methyl)amino)pyrimidin-4- |
| | yl)amino)-1-methyl-2-oxo-1,2-dihydroquinolin-3-yl)oxy)-N- |
| | methylacetamide |
| 298 | 2-((6-((5-chloro-2-(4-((2-(3-(2,6-dioxopiperidin-3-yl)-1-methyl-1H- |
| | indazol-6-yl)-2-azaspiro[3.5]nonan-7-yl)methyl)piperazin-1- |
| | yl)pyrimidin-4-yl)amino)-1-isopropyl-2-oxo-1,2-dihydroquinolin-3- |
| | yl)oxy)-N-methylacetamide |
| 299 | 2-[[6-[[5-chloro-2-[3-[[[3-(2,6-dioxo-3-piperidyl)-1-methyl-indazol-7- |
| | yl]-methyl-amino]methyl]-1-piperidyl]pyrimidin-4-yl]amino]-1-methyl- |
| | 2-oxo-3-quinolyl]oxy]-N-methyl-acetamide |
| 299a | 2-((6-((5-chloro-2-((3R)-3-(((3-(2,6-dioxopiperidin-3-yl)-1-methyl-1H- |
| | indazol-7-yl)(methyl)amino)methyl)piperidin-1-yl)pyrimidin-4- |
| | yl)amino)-1-methyl-2-oxo-1,2-dihydroquinolin-3-yl)oxy)-N- |
| | methylacetamide |
| 300 | 2-[[6-[[5-chloro-2-[2-[[[3-(2,6-dioxo-3-piperidyl)-1-methyl-indazol-7- |
| | yl]amino]methyl]pyrrolidin-1-yl]pyrimidin-4-yl]amino]-1-methyl-2- |
| | oxo-3-quinolyl]oxy]-N-methyl-acetamide |
| 300a | 2-((6-((5-chloro-2-((2R)-2-(((3-(2,6-dioxopiperidin-3-yl)-1-methyl-1H- |
| | indazol-7-yl)amino)methyl)pyrrolidin-1-yl)pyrimidin-4-yl)amino)-1- |
| | methyl-2-oxo-1,2-dihydroquinolin-3-yl)oxy)-N-methylacetamide |
| 300b | 2-((6-((5-chloro-2-((2S)-2-(((3-(2,6-dioxopiperidin-3-yl)-1-methyl-1H- |
| | indazol-7-yl)amino)methyl)pyrrolidin-1-yl)pyrimidin-4-yl)amino)-1- |
| | methyl-2-oxo-1,2-dihydroquinolin-3-yl)oxy)-N-methylacetamide |
| 301 | 2-[[6-[[5-chloro-2-[3-[[3-(2,6-dioxo-3-piperidyl)-1-methyl-indazol-7- |
| | yl]oxymethyl]-1-piperidyl]pyrimidin-4-yl]amino]-1-methyl-2-oxo-3- |
| | quinolyl]oxy]-N-methyl-acetamide |
| 301a | 2-((6-((5-chloro-2-((3S)-3-(((3-(2,6-dioxopiperidin-3-yl)-1-methyl-1H- |
| | indazol-7-yl)oxy)methyl)piperidin-1-yl)pyrimidin-4-yl)amino)-1- |
| | methyl-2-oxo-1,2-dihydroquinolin-3-yl)oxy)-N-methylacetamide |
| 301b | 2-((6-((5-chloro-2-((3R)-3-(((3-(2,6-dioxopiperidin-3-yl)-1-methyl-1H- |
| | indazol-7-yl)oxy)methyl)piperidin-1-yl)pyrimidin-4-yl)amino)-1- |
| | methyl-2-oxo-1,2-dihydroquinolin-3-yl)oxy)-N-methylacetamide |
| 302 | 2-[[6-[[5-chloro-2-[3-[[3-(2,6-dioxo-3-piperidyl)-1-methyl-indazol-6- |
| | yl]oxymethyl]-1-piperidyl]pyrimidin-4-yl]amino]-1-methyl-2-oxo-3- |
| | quinolyl]oxy]-N-methyl-acetamide |
| 302a | 2-((6-((5-chloro-2-((3S)-3-(((3-(2,6-dioxopiperidin-3-yl)-1-methyl-1H- |
| | indazol-6-yl)oxy)methyl)piperidin-1-yl)pyrimidin-4-yl)amino)-1- |
| | methyl-2-oxo-1,2-dihydroquinolin-3-yl)oxy)-N-methylacetamide |
| 302b | 2-((6-((5-chloro-2-((3R)-3-(((3-(2,6-dioxopiperidin-3-yl)-1-methyl-1H- |
| | indazol-6-yl)oxy)methyl)piperidin-1-yl)pyrimidin-4-yl)amino)-1- |
| | methyl-2-oxo-1,2-dihydroquinolin-3-yl)oxy)-N-methylacetamide |
| 303 | 2-[[6-[[5-chloro-2-[[1-[3-(2,6-dioxo-3-piperidyl)-1-methyl-indazol-7- |
| | yl]-3-piperidyl]methylamino]pyrimidin-4-yl]amino]-1-methyl-2-oxo-3- |
| | quinolyl]oxy]-N-methyl-acetamide |
| 303a | 2-((6-((5-chloro-2-((((3S)-1-(3-(2,6-dioxopiperidin-3-yl)-1-methyl-1H- |
| | indazol-7-yl)piperidin-3-yl)methyl)amino)pyrimidin-4-yl)amino)-1- |
| | methyl-2-oxo-1,2-dihydroquinolin-3-yl)oxy)-N-methylacetamide |
| 303b | 2-((6-((5-chloro-2-((((3R)-1-(3-(2,6-dioxopiperidin-3-yl)-1-methyl-1H- |
| | indazol-7-yl)piperidin-3-yl)methyl)amino)pyrimidin-4-yl)amino)-1- |
| | methyl-2-oxo-1,2-dihydroquinolin-3-yl)oxy)-N-methylacetamide |
| 304 | 2-[[6-[[5-chloro-2-[[4-[[3-(2,6-dioxo-3-piperidyl)-1-methyl-indazol-7- |
| | yl]amino]cyclohexyl]methylamino]pyrimidin-4-yl]amino]-1-methyl-2- |
| | oxo-3-quinolyl]oxy]-N-methyl-acetamide |
| 304a | 2-((6-((5-chloro-2-((((1s,4s)-4-((3-(2,6-dioxopiperidin-3-yl)-1-methyl- |
| | 1H-indazol-7-yl)amino)cyclohexyl)methyl)amino)pyrimidin-4- |
| | yl)amino)-1-methyl-2-oxo-1,2-dihydroquinolin-3-yl)oxy)-N- |
| | methylacetamide |
| 304b | 2-((6-((5-chloro-2-((((1r,4r)-4-((3-(2,6-dioxopiperidin-3-yl)-1-methyl- |
| | 1H-indazol-7-yl)amino)cyclohexyl)methyl)amino)pyrimidin-4- |
| | yl)amino)-1-methyl-2-oxo-1,2-dihydroquinolin-3-yl)oxy)-N- |
| | methylacetamide |
| 305 | 2-[[6-[[5-chloro-2-[4-[[1-[3-(2,6-dioxo-3-piperidyl)-1-methyl-indazol- |
| | 7-yl]pyrrolidin-3-yl]methyl]piperazin-1-yl]pyrimidin-4-yl]amino]-1- |
| | methyl-2-oxo-3-quinolyl]oxy]-N-methyl-acetamide |
| 305a | 2-((6-((5-chloro-2-(4-(((3R)-1-(3-(2,6-dioxopiperidin-3-yl)-1-methyl- |
| | 1H-indazol-7-yl)pyrrolidin-3-yl)methyl)piperazin-1-yl)pyrimidin-4- |
| | yl)amino)-1-methyl-2-oxo-1,2-dihydroquinolin-3-yl)oxy)-N- |
| | methylacetamide |
| 305b | 2-((6-((5-chloro-2-(4-(((3S)-1-(3-(2,6-dioxopiperidin-3-yl)-1-methyl- |
| | 1H-indazol-7-yl)pyrrolidin-3-yl)methyl)piperazin-1-yl)pyrimidin-4- |
| | yl)amino)-1-methyl-2-oxo-1,2-dihydroquinolin-3-yl)oxy)-N- |
| | methylacetamide |
| 306 | 2-[[6-[[5-chloro-2-[4-[[1-[3-(2,6-dioxo-3-piperidyl)-1-methyl-indazol- |
| | 6-yl]pyrrolidin-3-yl]methyl]piperazin-1-yl]pyrimidin-4-yl]amino]-1- |
| | methyl-2-oxo-3-quinolyl]oxy]-N-methyl-acetamide |
| 306a | 2-((6-((5-chloro-2-(4-(((3R)-1-(3-(2,6-dioxopiperidin-3-yl)-1-methyl- |
| | 1H-indazol-6-yl)pyrrolidin-3-yl)methyl)piperazin-1-yl)pyrimidin-4- |
| | yl)amino)-1-methyl-2-oxo-1,2-dihydroquinolin-3-yl)oxy)-N- |
| | methylacetamide |
| 306b | 2-((6-((5-chloro-2-(4-(((3S)-1-(3-(2,6-dioxopiperidin-3-yl)-1-methyl- |
| | 1H-indazol-6-yl)pyrrolidin-3-yl)methyl)piperazin-1-yl)pyrimidin-4- |
| | yl)amino)-1-methyl-2-oxo-1,2-dihydroquinolin-3-yl)oxy)-N- |
| | methylacetamide |
| 307 | 2-[[6-[[5-chloro-2-[4-[1-[1-[3-(2,6-dioxo-3-piperidyl)-1-methyl- |
| | indazol-7-yl]-4-piperidyl]ethyl]piperazin-1-yl]pyrimidin-4-yl]amino]-1- |
| | methyl-2-oxo-3-quinolyl]oxy]-N-methyl-acetamide |
| 307a | rel-2-((6-((5-chloro-2-(4-((1S)-1-(1-(3-(2,6-dioxopiperidin-3-yl)-1- |
| | methyl-1H-indazol-7-yl)piperidin-4-yl)ethyl)piperazin-1-yl)pyrimidin- |
| | 4-yl)amino)-1-methyl-2-oxo-1,2-dihydroquinolin-3-yl)oxy)-N- |
| | methylacetamide |
| 307b | rel-2-((6-((5-chloro-2-(4-((1R)-1-(1-(3-(2,6-dioxopiperidin-3-yl)-1- |
| | methyl-1H-indazol-7-yl)piperidin-4-yl)ethyl)piperazin-1-yl)pyrimidin- |
| | 4-yl)amino)-1-methyl-2-oxo-1,2-dihydroquinolin-3-yl)oxy)-N- |
| | methylacetamide |
| 308 | 2-[[6-[[5-chloro-2-[4-[[1-[3-(2,6-dioxo-3-piperidyl)-1-methyl-indazol- |
| | 7-yl]-4-piperidyl]methyl]-3-methyl-piperazin-1-yl]pyrimidin-4- |
| | yl]amino]-1-methyl-2-oxo-3-quinolyl]oxy]-N-methyl-acetamide |
| 308a | 2-((6-((5-chloro-2-((3R)-4-((1-(3-(2,6-dioxopiperidin-3-yl)-1-methyl- |
| | 1H-indazol-7-yl)piperidin-4-yl)methyl)-3-methylpiperazin-1- |
| | yl)pyrimidin-4-yl)amino)-1-methyl-2-oxo-1,2-dihydroquinolin-3- |
| | yl)oxy)-N-methylacetamide |
| 308b | 2-((6-((5-chloro-2-((3S)-4-((1-(3-(2,6-dioxopiperidin-3-yl)-1-methyl- |
| | 1H-indazol-7-yl)piperidin-4-yl)methyl)-3-methylpiperazin-1- |
| | yl)pyrimidin-4-yl)amino)-1-methyl-2-oxo-1,2-dihydroquinolin-3- |
| | yl)oxy)-N-methylacetamide |
| 309 | 2-[[6-[[5-chloro-2-[4-[[1-[3-(2,6-dioxo-3-piperidyl)-1-methyl-indazol- |
| | 6-yl]-4-piperidyl]methyl]-3-methyl-piperazin-1-yl]pyrimidin-4- |
| | yl]amino]-1-methyl-2-oxo-3-quinolyl]oxy]-N-methyl-acetamide |
| 309a | 2-((6-((5-chloro-2-((3R)-4-((1-(3-(2,6-dioxopiperidin-3-yl)-1-methyl- |
| | 1H-indazol-6-yl)piperidin-4-yl)methyl)-3-methylpiperazin-1- |
| | yl)pyrimidin-4-yl)amino)-1-methyl-2-oxo-1,2-dihydroquinolin-3- |
| | yl)oxy)-N-methylacetamide |
| 309b | 2-((6-((5-chloro-2-((3S)-4-((1-(3-(2,6-dioxopiperidin-3-yl)-1-methyl- |
| | 1H-indazol-6-yl)piperidin-4-yl)methyl)-3-methylpiperazin-1- |
| | yl)pyrimidin-4-yl)amino)-1-methyl-2-oxo-1,2-dihydroquinolin-3- |
| | yl)oxy)-N-methylacetamide |
| 310 | 2-((6-((5-chloro-2-(4-((1-(3-(2,6-dioxopiperidin-3-yl)-1-methyl-1H- |
| | indazol-7-yl)azetidin-3-yl)methyl)piperidin-1-yl)pyrimidin-4-yl)amino)- |
| | 1-methyl-2-oxo-1,2-dihydroquinolin-3-yl)oxy)-N-methylacetamide |
| 311 | 2-((6-((5-chloro-2-(4-((2-(3-(2,6-dioxopiperidin-3-yl)-1-methyl-1H- |
| | indazol-7-yl)-2-azaspiro[3.5]nonan-7-yl)methyl)piperazin-1- |
| | yl)pyrimidin-4-yl)amino)-1-isopropyl-2-oxo-1,2-dihydroquinolin-3- |
| | yl)oxy)-N-methylacetamide |
| 312 | 2-[[6-[[5-chloro-2-[3-[[[3-(2,6-dioxo-3-piperidyl)-1-methyl-indazol-6- |
| | yl]-methyl-amino]methyl]-1-piperidyl]pyrimidin-4-yl]amino]-1-methyl- |
| | 2-oxo-3-quinolyl]oxy]-N-methyl-acetamide |
| 312a | 2-((6-((5-chloro-2-((3R)-3-(((3-(2,6-dioxopiperidin-3-yl)-1-methyl-1H- |
| | indazol-6-yl)(methyl)amino)methyl)piperidin-1-yl)pyrimidin-4- |
| | yl)amino)-1-methyl-2-oxo-1,2-dihydroquinolin-3-yl)oxy)-N- |
| | methylacetamide |
| 313 | 2-[[6-[[5-chloro-2-[3-[[3-(2,6-dioxo-3-piperidyl)-1-methyl-indazol-6- |
| | yl]oxymethyl]pyrrolidin-1-yl]pyrimidin-4-yl]amino]-1-methyl-2-oxo-3- |
| | quinolyl]oxy]-N-methyl-acetamide |
| 313a | 2-((6-((5-chloro-2-((3S)-3-(((3-(2,6-dioxopiperidin-3-yl)-1-methyl-1H- |
| | indazol-6-yl)oxy)methyl)pyrrolidin-1-yl)pyrimidin-4-yl)amino)-1- |
| | methyl-2-oxo-1,2-dihydroquinolin-3-yl)oxy)-N-methylacetamide |
| 314 | 2-((6-((5-chloro-2-(4-((4-(3-(2,6-dioxopiperidin-3-yl)benzoyl)piperazin- |
| | 1-yl)methyl)piperidin-1-yl)pyrimidin-4-yl)amino)-1-methyl-2-oxo-1,2- |
| | dihydroquinolin-3-yl)oxy)-N-methylacetamide |
| 315 | 2-((6-((5-chloro-2-((4-(((3-(2,6-dioxopiperidin-3-yl)-1-methyl-1H- |
| | indazol-6-yl)amino)methyl)benzyl)amino)pyrimidin-4-yl)amino)-1- |
| | methyl-2-oxo-1,2-dihydroquinolin-3-yl)oxy)-N-methylacetamide |
| 316 | 2-((6-((5-chloro-2-(4-((1-(3-(2,6-dioxopiperidin-3-yl)-1-methyl-1H- |
| | indazol-7-yl)piperidin-4-yl)oxy)piperidin-1-yl)pyrimidin-4-yl)amino)-1- |
| | methyl-2-oxo-1,2-dihydroquinolin-3-yl)oxy)-N-methylacetamide |
| 317 | 2-[[6-[[5-chloro-2-[2-[[3-(2,6-dioxo-3-piperidyl)-1-methyl-indazol-6- |
| | yl]oxymethyl]morpholin-4-yl]pyrimidin-4-yl]amino]-1-methyl-2-oxo-3- |
| | quinolyl]oxy]-N-methyl-acetamide |
| 317a | 2-((6-((5-chloro-2-((2S)-2-(((3-(2,6-dioxopiperidin-3-yl)-1-methyl-1H- |
| | indazol-6-yl)oxy)methyl)morpholino)pyrimidin-4-yl)amino)-1-methyl- |
| | 2-oxo-1,2-dihydroquinolin-3-yl)oxy)-N-methylacetamide |
| 318 | 2-((6-((5-chloro-2-((4-(((3-(2,6-dioxopiperidin-3-yl)-1-methyl-1H- |
| | indazol-7-yl)amino)methyl)benzyl)amino)pyrimidin-4-yl)amino)-1- |
| | methyl-2-oxo-1,2-dihydroquinolin-3-yl)oxy)-N-methylacetamide |
| 319 | 2-((6-((5-chloro-2-(4-(4-((3-(2,6-dioxopiperidin-3-yl)-1-methyl-1H- |
| | indazol-6-yl)oxy)butyl)piperidin-1-yl)pyrimidin-4-yl)amino)-1-methyl- |
| | 2-oxo-1,2-dihydroquinolin-3-yl)oxy)-N-methylacetamide |
| 320 | 2-((6-((5-chloro-2-(4-((1-(3-(2,6-dioxopiperidin-3-yl)-1-methyl-1H- |
| | indazol-7-yl)pyrrolidin-3-yl)methyl)piperidin-1-yl)pyrimidin-4- |
| | yl)amino)-1-methyl-2-oxo-1,2-dihydroquinolin-3-yl)oxy)-N- |
| | methylacetamide |
| 321 | 2-((6-((5-chloro-2-(4-((1-(3-(2,6-dioxopiperidin-3-yl)-1-methyl-1H- |
| | indazole-6-carbonyl)piperidin-4-yl)methyl)piperazin-1-yl)pyrimidin-4- |
| | yl)amino)-1-methyl-2-oxo-1,2-dihydroquinolin-3-yl)oxy)-N- |
| | methylacetamide |
| 322 | 2-((6-((5-chloro-2-(4-((4-(3-(2,6-dioxopiperidin-3-yl)-1-methyl-1H- |
| | indazole-6-carbonyl)piperazin-1-yl)methyl)piperidin-1-yl)pyrimidin-4- |
| | yl)amino)-1-methyl-2-oxo-1,2-dihydroquinolin-3-yl)oxy)-N- |
| | methylacetamide |
| 323 | 2-((6-((5-chloro-2-(4-((1-(3-(2,6-dioxopiperidin-3-yl)-1-methyl-1H- |
| | indazole-7-carbonyl)piperidin-4-yl)methyl)piperazin-1-yl)pyrimidin-4- |
| | yl)amino)-1-methyl-2-oxo-1,2-dihydroquinolin-3-yl)oxy)-N- |
| | methylacetamide |
| 324 | 2-((6-((5-chloro-2-(4-((4-(3-(2,6-dioxopiperidin-3-yl)-1-methyl-1H- |
| | indazole-7-carbonyl)piperazin-1-yl)methyl)piperidin-1-yl)pyrimidin-4- |
| | yl)amino)-1-methyl-2-oxo-1,2-dihydroquinolin-3-yl)oxy)-N- |
| | methylacetamide |
| 325 | 2-((6-((5-chloro-2-(3-((1-(3-(2,6-dioxopiperidin-3-yl)-1-methyl-1H- |
| | indazol-7-yl)piperidin-4-yl)methyl)-3,8-diazabicyclo[3.2.1]octan-8- |
| | yl)pyrimidin-4-yl)amino)-1-methyl-2-oxo-1,2-dihydroquinolin-3- |
| | yl)oxy)-N-methylacetamide |
| 326 | 2-((6-((5-chloro-2-(2-(3-(2,6-dioxopiperidin-3-yl)-1-methyl-1H- |
| | indazol-7-yl)octahydro-5H-pyrrolo[3,4-c]pyridin-5-yl)pyrimidin-4- |
| | yl)amino)-1-methyl-2-oxo-1,2-dihydroquinolin-3-yl)oxy)-N- |
| | methylacetamide |
| 327 | 2-((6-((5-chloro-2-(4-(4-(3-(2,6-dioxopiperidin-3-yl)-1-methyl-1H- |
| | indazol-7-yl)piperazine-1-carbonyl)piperidin-1-yl)pyrimidin-4- |
| | yl)amino)-1-methyl-2-oxo-1,2-dihydroquinolin-3-yl)oxy)-N- |
| | methylacetamide |
| 328 | 2-((6-((5-chloro-2-(4-(4-(3-(2,6-dioxopiperidin-3-yl)-1-methyl-1H- |
| | indazol-6-yl)piperazine-1-carbonyl)piperidin-1-yl)pyrimidin-4- |
| | yl)amino)-1-methyl-2-oxo-1,2-dihydroquinolin-3-yl)oxy)-N- |
| | methylacetamide |
| 329 | 2-((6-((5-chloro-2-(2-(3-(2,6-dioxopiperidin-3-yl)-1-methyl-1H- |
| | indazol-6-yl)-2,6-diazaspiro[3.5]nonan-6-yl)pyrimidin-4-yl)amino)-1- |
| | methyl-2-oxo-1,2-dihydroquinolin-3-yl)oxy)-N-methylacetamide |
| 330 | 2-((6-((5-chloro-2-(8-(3-(2,6-dioxopiperidin-3-yl)-1-methyl-1H- |
| | indazol-7-yl)-2,8-diazaspiro[5.5]undecan-2-yl)pyrimidin-4-yl)amino)-1- |
| | methyl-2-oxo-1,2-dihydroquinolin-3-yl)oxy)-N-methylacetamide |
| 331 | 2-((6-((5-chloro-2-(2-(3-(2,6-dioxopiperidin-3-yl)-1-methyl-1H- |
| | indazol-6-yl)-2,7-diazaspiro[4.5]decan-7-yl)pyrimidin-4-yl)amino)-1- |
| | methyl-2-oxo-1,2-dihydroquinolin-3-yl)oxy)-N-methylacetamide |
| 332 | 2-((6-((5-chloro-2-(4-((1-(3-(2,6-dioxopiperidin-3-yl)-1-methyl-1H- |
| | indazol-6-yl)pyrrolidin-3-yl)methyl)piperidin-1-yl)pyrimidin-4- |
| | yl)amino)-1-methyl-2-oxo-1,2-dihydroquinolin-3-yl)oxy)-N- |
| | methylacetamide |
| 333 | 2-((6-((5-chloro-2-(4-((4-(3-(2,6-dioxopiperidin-3-yl)-1-methyl-1H- |
| | indazol-7-yl)piperazin-1-yl)methyl)piperidin-1-yl)pyrimidin-4- |
| | yl)amino)-1-methyl-2-oxo-1,2-dihydroquinolin-3-yl)oxy)-N- |
| | methylacetamide |
| 334 | 2-((6-((5-chloro-2-(4-((4-(3-(2,6-dioxopiperidin-3-yl)-1-methyl-1H- |
| | indazol-6-yl)piperazin-1-yl)methyl)piperidin-1-yl)pyrimidin-4- |
| | yl)amino)-1-methyl-2-oxo-1,2-dihydroquinolin-3-yl)oxy)-N- |
| | methylacetamide |
| 335 | 2-((6-((5-chloro-2-(8-(3-(2,6-dioxopiperidin-3-yl)-1-methyl-1H- |
| | indazol-6-yl)-2,8-diazaspiro[5.5]undecan-2-yl)pyrimidin-4-yl)amino)-1- |
| | methyl-2-oxo-1,2-dihydroquinolin-3-yl)oxy)-N-methylacetamide |
| 336 | 2-[[6-[[5-chloro-2-[4-[[1-[3-(2,6-dioxo-3-piperidyl)-1-methyl-indazol- |
| | 7-yl]-4-piperidyl]methyl]-3,5-dimethyl-piperazin-1-yl]pyrimidin-4- |
| | yl]amino]-1-methyl-2-oxo-3-quinolyl]oxy]-N-methyl-acetamide |
| 336a | 2-((6-((5-chloro-2-((3S,5R)-4-((1-(3-(2,6-dioxopiperidin-3-yl)-1- |
| | methyl-1H-indazol-7-yl)piperidin-4-yl)methyl)-3,5-dimethylpiperazin- |
| | 1-yl)pyrimidin-4-yl)amino)-1-methyl-2-oxo-1,2-dihydroquinolin-3- |
| | yl)oxy)-N-methylacetamide |
| 337 | 2-((6-((5-chloro-2-(4-(2-(1-(3-(2,6-dioxopiperidin-3-yl)-1-methyl-1H- |
| | indazol-7-yl)piperidin-4-yl)propan-2-yl)piperazin-1-yl)pyrimidin-4- |
| | yl)amino)-1-methyl-2-oxo-1,2-dihydroquinolin-3-yl)oxy)-N- |
| | methylacetamide |
| 338 | 2-((6-((5-chloro-2-(5-((3-(2,6-dioxopiperidin-3-yl)-1-methyl-1H- |
| | indazol-6-yl)amino)octahydro-2H-isoindol-2-yl)pyrimidin-4-yl)amino)- |
| | 1-methyl-2-oxo-1,2-dihydroquinolin-3-yl)oxy)-N-methylacetamide |
| 339 | 2-((6-((5-chloro-2-(5-((3-(2,6-dioxopiperidin-3-yl)-1-methyl-1H- |
| | indazol-7-yl)amino)octahydro-2H-isoindol-2-yl)pyrimidin-4-yl)amino)- |
| | 1-methyl-2-oxo-1,2-dihydroquinolin-3-yl)oxy)-N-methylacetamide |
| 340 | 2-((6-((5-chloro-2-(4-(2-(1-(3-(2,6-dioxopiperidin-3-yl)-1-methyl-1H- |
| | indazol-6-yl)piperidin-4-yl)propan-2-yl)piperazin-1-yl)pyrimidin-4- |
| | yl)amino)-1-methyl-2-oxo-1,2-dihydroquinolin-3-yl)oxy)-N- |
| | methylacetamide |
| 341 | 2-((6-((5-chloro-2-(7-((3-(2,6-dioxopiperidin-3-yl)-1-methyl-1H- |
| | indazol-6-yl)amino)-2-azaspiro[3.5]nonan-2-yl)pyrimidin-4-yl)amino)- |
| | 1-methyl-2-oxo-1,2-dihydroquinolin-3-yl)oxy)-N-methylacetamide |
| 342 | 2-((6-((5-chloro-2-(7-((3-(2,6-dioxopiperidin-3-yl)-1-methyl-1H- |
| | indazol-7-yl)amino)-2-azaspiro[3.5]nonan-2-yl)pyrimidin-4-yl)amino)- |
| | 1-methyl-2-oxo-1,2-dihydroquinolin-3-yl)oxy)-N-methylacetamide |
| 343 | 2-((6-((5-chloro-2-(8-((3-(2,6-dioxopiperidin-3-yl)-1-methyl-1H- |
| | indazol-6-yl)amino)-2-azaspiro[4.5]decan-2-yl)pyrimidin-4-yl)amino)- |
| | 1-methyl-2-oxo-1,2-dihydroquinolin-3-yl)oxy)-N-methylacetamide |
| 344 | 2-((6-((5-chloro-2-(8-((3-(2,6-dioxopiperidin-3-yl)-1-methyl-1H- |
| | indazol-7-yl)amino)-2-azaspiro[4.5]decan-2-yl)pyrimidin-4-yl)amino)- |
| | 1-methyl-2-oxo-1,2-dihydroquinolin-3-yl)oxy)-N-methylacetamide |
| 345 | 2-((6-((5-chloro-2-(4-(1-(3-(2,6-dioxopiperidin-3-yl)-1-methyl-1H- |
| | indazol-7-yl)piperidine-4-carbonyl)piperazin-1-yl)pyrimidin-4- |
| | yl)amino)-1-methyl-2-oxo-1,2-dihydroquinolin-3-yl)oxy)-N- |
| | methylacetamide |
| 346 | 2-((6-((5-chloro-2-(4-((1-(3-(2,6-dioxopiperidin-3-yl)-1-methyl-1H- |
| | indazol-7-yl)piperidin-4-yl)methyl)piperazin-1-yl)pyridin-4-yl)amino)- |
| | 1-methyl-2-oxo-1,2-dihydroquinolin-3-yl)oxy)-N-methylacetamide |
| 347 | 3-[7-[4-[[4-[4-[(3-acetonyloxy-1-methyl-2-oxo-6-quinolyl)amino]-5- |
| | chloro-pyrimidin-2-yl]-2-methyl-piperazin-1-yl]methyl]-1-piperidyl]-1- |
| | methyl-indazol-3-yl]piperidine-2,6-dione |
| 347a | 3-(7-(4-(((S)-4-(5-chloro-4-((1-methyl-2-oxo-3-(2-oxopropoxy)-1,2- |
| | dihydroquinolin-6-yl)amino)pyrimidin-2-yl)-2-methylpiperazin-1- |
| | yl)methyl)piperidin-1-yl)-1-methyl-1H-indazol-3-yl)piperidine-2,6- |
| | dione |
| 348 | 3-(6-(4-((9-(5-chloro-4-((1-methyl-2-oxo-3-(2-oxopropoxy)-1,2- |
| | dihydroquinolin-6-yl)amino)pyrimidin-2-yl)-3,9- |
| | diazaspiro[5.5]undecan-3-yl)methyl)piperidin-1-yl)-1-methyl-1H- |
| | indazol-3-yl)piperidine-2,6-dione |

Certain examples of Formula (I) compounds were synthesized using methods involving resolution of stereoisomeric mixture(s) (e.g., SFC separation of stereoisomers). In Table C1, the resolved stereogenic centers in these compounds are labelled with the “or1” and/or “or2” enhanced stereochemical notations. In some instances, the stereoisomeric resolutions were performed during the last step of the synthesis, thereby providing the individual stereoisomers of the Formula (I) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (I) compounds as separate stereoisomers. Methods of resolution and correlation between resolved intermediates and Formula (I) compounds are disclosed in the examples herein and in Examples 28, 39, or 83. A person of ordinary skill in the art would understand that, under either approach for stereoisomeric resolution, stereoisomers having both (R)- and (S)-configurations at a resolved stereogenic center are provided. See Table C3, wherein Table C1 compounds whose stereoisomers contain the or1 and/or or2 stereochemical notations are provided in non-stereogenic form, followed by the respective stereoisomers having the (R)- and (S)-configurations.

TABLE C3

| No. | Compound Structure |
|---|---|
| 290 | 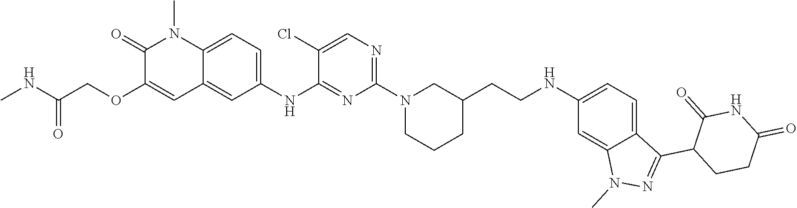 [SMILES: CNC(=O)COc1cc2cc(Nc3nc(N4CCCC(CCNc5ccc6c(C7CCC(=O)NC7=O)nn(C)c6c5)C4)ncc3Cl)ccc2n(C)c1=O] |
| | 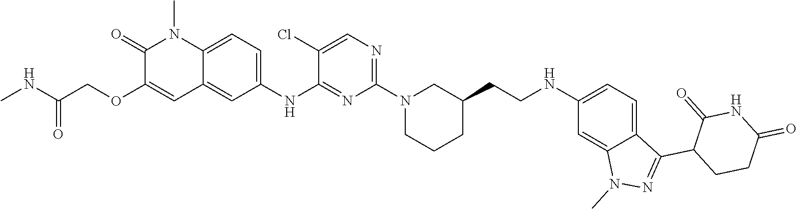 [SMILES: CNC(=O)COc1cc2cc(Nc3nc(N4CCC[C@H](CCNc5ccc6c(C7CCC(=O)NC7=O)nn(C)c6c5)C4)ncc3Cl)ccc2n(C)c1=O] |
| | 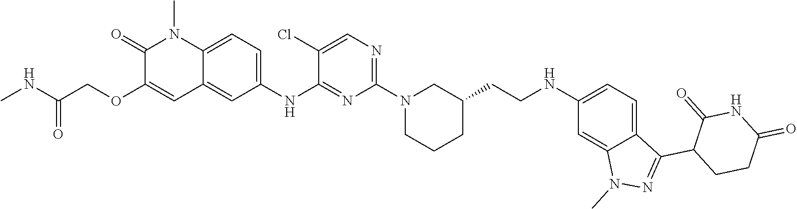 [SMILES: CNC(=O)COc1cc2cc(Nc3nc(N4CCC[C@@H](CCNc5ccc6c(C7CCC(=O)NC7=O)nn(C)c6c5)C4)ncc3Cl)ccc2n(C)c1=O] |
| 294 | 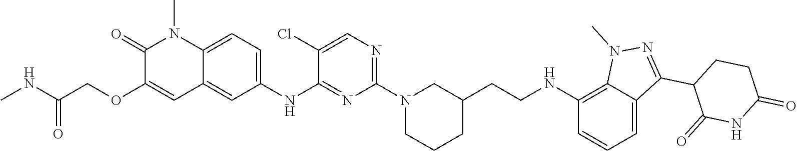 [SMILES: CNC(=O)COc1cc2cc(Nc3nc(N4CCCC(CCNc5cccc6c(C7CCC(=O)NC7=O)nn(C)c56)C4)ncc3Cl)ccc2n(C)c1=O] |
| | 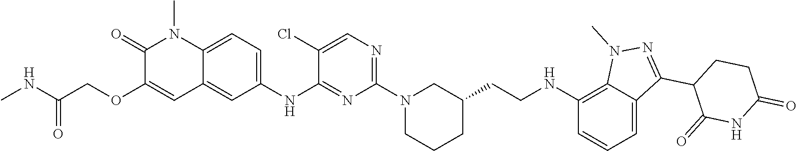 [SMILES: CNC(=O)COc1cc2cc(Nc3nc(N4CCC[C@@H](CCNc5cccc6c(C7CCC(=O)NC7=O)nn(C)c56)C4)ncc3Cl)ccc2n(C)c1=O] |
| | 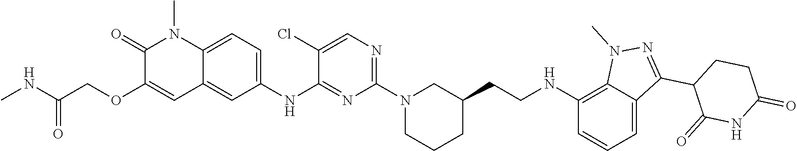 [SMILES: CNC(=O)COc1cc2cc(Nc3nc(N4CCC[C@H](CCNc5cccc6c(C7CCC(=O)NC7=O)nn(C)c56)C4)ncc3Cl)ccc2n(C)c1=O] |
| 307 | 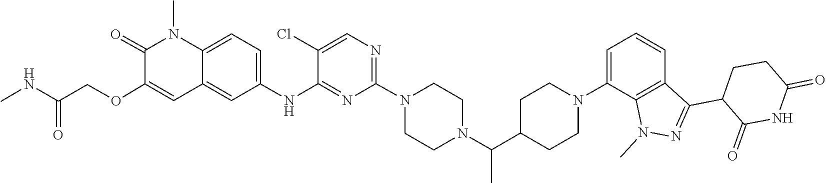 [SMILES: CNC(=O)COc1cc2cc(Nc3nc(N4CCN(C(C)C5CCN(c6cccc7c(C8CCC(=O)NC8=O)nn(C)c67)CC5)CC4)ncc3Cl)ccc2n(C)c1=O] |
| | 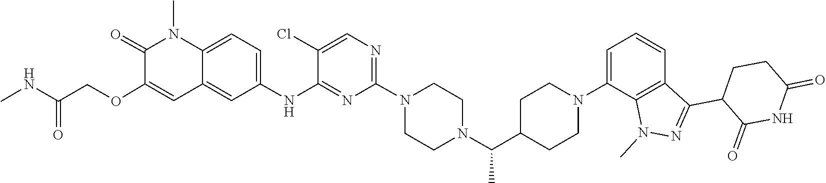 [SMILES: CNC(=O)COc1cc2cc(Nc3nc(N4CCN([C@@H](C)C5CCN(c6cccc7c(C8CCC(=O)NC8=O)nn(C)c67)CC5)CC4)ncc3Cl)ccc2n(C)c1=O] |
| | 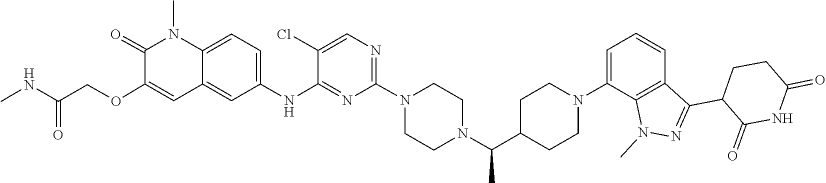 [SMILES: CNC(=O)COc1cc2cc(Nc3nc(N4CCN([C@H](C)C5CCN(c6cccc7c(C8CCC(=O)NC8=O)nn(C)c67)CC5)CC4)ncc3Cl)ccc2n(C)c1=O] |

Exemplary compounds of Formula (I) also include those depicted in Table C1 of U.S. Provisional Application Ser. No. 63/420,411, filed Oct. 28, 2022; Table C1 of U.S. Provisional Application Ser. No. 63/444,778, filed Feb. 10, 2023; Table C1 of U.S. Provisional Application Ser. No. 63/497,063, filed Apr. 19, 2023; and Table C1 of U.S. Provisional Application Ser. No. 63/501,082, filed May 9, 2023; or pharmaceutically acceptable salts thereof, wherein each Table C1 is incorporated herein by reference in its entirety.

Also provided herein are compounds of Formula (II):

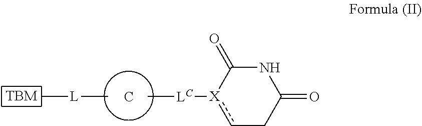

or a pharmaceutically acceptable salt thereof, wherein:
TBM is selected from the group consisting of (T1), (T2), and (T3):

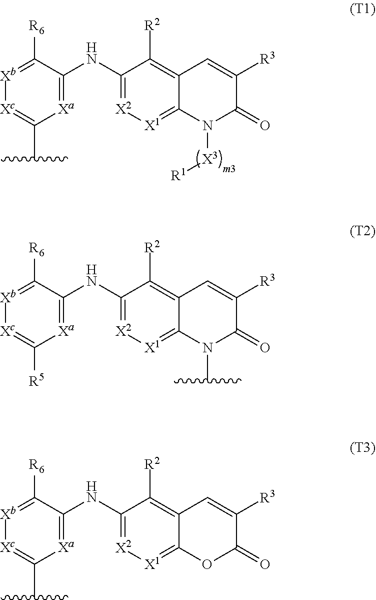

X¹ and X² are independently selected from the group consisting of: N and CR²;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
m3 is 0, 1, 2, or 3;
each X³ is independently selected from the group consisting of: —O—, —NR^{f}—, —C(═O)—, and C₁₋₃ alkylene optionally substituted with 1-3 R^{c};
R¹ is selected from the group consisting of: H, halo, cyano, and R^{b1};
provided that the N—(X³)ₘ₃—R¹ moiety does not contain any O—O, N—O, or N-halo bonds;
R³ is selected from the group consisting of:
-A¹-C(R⁴R⁴)-A²; and
—CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃-6 cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂(C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1};
X^{a} and X^{c} are independently selected from the group consisting of: N, CH, and CF, provided that one or both of X^{a} and X^{c} is N;
X^{b} is selected from the group consisting of N and CR^{x1},
R⁶ and R^{x1} are each independently selected from the group consisting of: H, halo, C₁₋₂ alkyl, C₁₋₂ haloalkyl, C₁₋₂ alkoxy, CN, and —C≡CH;
R⁵ is selected from the group consisting of:
a) H;
b) halo;
c) cyano;
d) NO₂;
e) C₁₋₆ alkyl, C₂₋₆ alkenyl, or C₂₋₆ alkynyl, each optionally substituted with 1-6 R^{c};
f) C₁₋₆ alkoxy optionally substituted with 1-6 R^{c}; and
g) -L⁵¹-R⁵¹;
L51 is selected from the group consisting of: a bond, O, S(O)₀₋₂, C(O), C(O)O*, OC(O)*, C(O)N(R^{f})*, N(R^{f}) C(O)*, N(R^{f}) C(O)N(R^{f}), N(R^{f}) C(O)O*, OC(O)N(R^{f})*, S(O)₂N(R^{f})*, N(R^{f})SO₂*, and —N(R⁴)—(C₀₋₂ alkylene)-*, wherein * represents the point of attachment to R⁵¹, and the C₀₋₂ alkylene portion of —N(R^{d})—(C₀₋₂ alkylene)-* is optionally substituted with 1-3 substituents independently selected from the group consisting of: C₁₋₃ alkyl and halo;
R⁵¹ is selected from the group consisting of:
a) H;
b) C₁₋₆ alkyl, C₂₋₆ alkenyl, or C₂₋₆ alkynyl, each optionally substituted with 1-6 R^{c} and further optionally substituted with R^{b1}, and
c) C₃₋₁₀ cycloalkyl, 4-12 membered heterocyclyl, C₆₋₁₀ aryl, or 5-15 membered heteroaryl, each of which is optionally substituted with 1-6 substituents independently selected from the group consisting of: R^{a} and R^{b}, and further optionally substituted with —C(O)N(R^{f})(CH₂CH₂O)₁₋₄—R^{a};
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1}, L^{A3}, and L^{A4}, provided that 1-3 occurrences of L^{A} is L^{A4}.

### (BB)

n1 is an integer from 0 to 20; and
each L^{A} is independently selected from the group consisting of: L^{A1} and L^{A3},
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, N—N, N—S(O)₀, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{e};
Ring C is selected from the group consisting of:

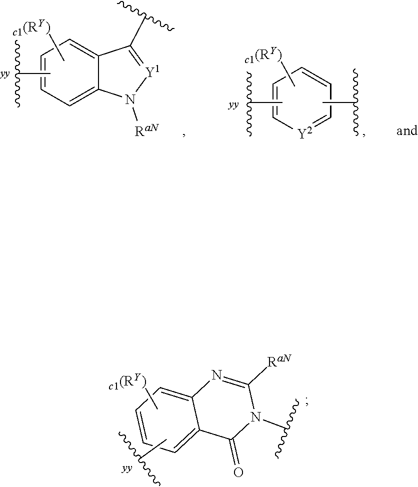

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{c}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂(C₁₋₆ alkyl);
(m) S(O)₀₋₂(C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1} wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(═O)C₁₋₆ haloalkyl, C(—O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂,
provided that when TBM is (T1), wherein X¹ and X² are independently selected CR²; and X is N, then Ring C is

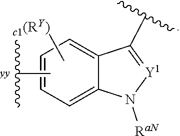

In Formula (II), L, Ring C, L^{C}, and X can be as defined herein for Formula (I).

Exemplary compounds of Formula (II) include those depicted in Table C1 of U.S. Provisional Application Ser. No. 63/351,715, filed Jun. 13, 2022; Table C1 of U.S. Provisional Application Ser. No. 63/351,697, filed Jun. 13, 2022; Table C1 of U.S. Provisional Application Ser. No. 63/395,630, filed Aug. 5, 2022; Table C1 of U.S. Provisional Application Ser. No. 63/395,638, filed Aug. 5, 2022; Table C1 of U.S. Provisional Application Ser. No. 63/420,421, filed Oct. 28, 2022; and Table C1 of U.S. Provisional Application Ser. No. 63/444,801, filed Feb. 10, 2023; or pharmaceutically acceptable salts thereof, wherein each Table C1 is incorporated herein by reference in its entirety.

In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, reduce cell viability in a cell line expressing a BCL6 protein with an EC₅₀ of less than 1 μM (e.g., less than 750 nM, less than 500 nM, or less than 200 nM). In some embodiments, the compounds reduce cell viability in a cell line expressing the BCL6 protein with an EC₅₀ of less than 200 nM (e.g., less than 150 nM, less than 200 nM, less than 100 nM, less than 10 nM, less than 1 nM). For example, the compounds can reduce cell viability in a cell line expressing the BCL6 protein with an EC₅₀ of about 0.1 nM to about 100 nM, about 0.1 nM to about 50 nM, about 1 nM to about 50 nM, about 1 nM to about 20 nM, or about 0.1 nM to about 1 nM.

In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a DC₅₀ of less than 1 μM (e.g., less than 750 nM, less than 500 nM, or less than 200 nM). In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a DC₅₀ of less than 200 nM (e.g., less than 150 nM, less than 200 nM, less than 100 nM, less than 10 nM, less than 1 nM). For example, the compounds can induce degradation of a BCL6 protein in a cell line expressing the BCL6 protein with a DC₅₀ of about 0.1 nM to about 100 nM, about 0.1 nM to about 50 nM, about 1 nM to about 50 nM, about 1 nM to about 20 nM, or about 0.1 nM to about 1 nM.

In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a Yₘᵢₙ of less than 70% (e.g., less than 50%, less than 30%, less than 20%, or less than 10%). In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a Yₘᵢₙ of less than 50% (e.g., less than 40%, less than 30%, less than 20%, less than 10%, or less than 5%). In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, induce degradation of a BCL6 protein in a cell line expressing the BCL6 protein with a Yₘᵢₙ of less than 30% (e.g., less than 25%, less than 20%, less than 15%, less than 10%, or less than 5%). For example, the compounds can induce degradation of a BCL6 protein in a cell line expressing the BCL6 protein with a Yₘᵢₙ of about 1% to about 70% (e.g., about 5% to about 50% or about 10% to about 30%).

Also provided herein is a BCL6 protein non-covalently bound with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Also provided herein is a ternary complex comprising a BCL6 protein, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and a CRBN protein or a portion thereof.

### Chemical Definitions

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

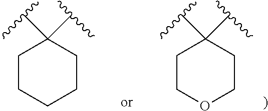 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms)) (e.g.,

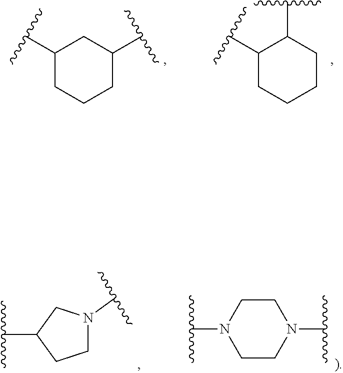 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Cycloalkyl may include multiple fused and/or bridged rings. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Cycloalkyl also includes spirocyclic rings (e.g., spirocyclic bicycle wherein two rings are connected through just one atom). Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

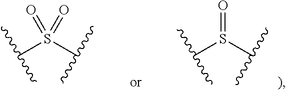

and P (inclusive of oxidized forms such as:

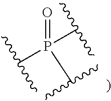 [SMILES: *P(*)(*)=O]

(e.g., N, O, and S (inclusive of oxidized forms such as:

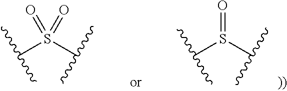

and at least one ring in the system is aromatic (but does not have to be a ring which contains a heteroatom, e.g. tetrahydroisoquinolinyl, e.g., tetrahydroquinolinyl). In some embodiments, heteroaryl groups contain 1-4 (e.g., 1, 2, or 3) ring heteroatoms each independently selected from the group consisting of N, O, and S (inclusive of oxidized forms such as:

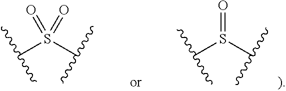

Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone (e.g.,

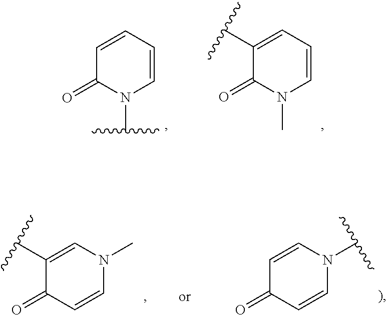

pyrimidone (e.g.,

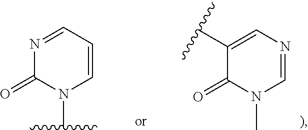

pyridazinone (e.g.,

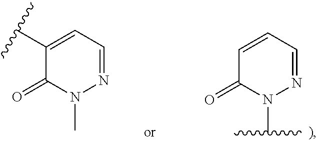

pyrazinone (e.g.,

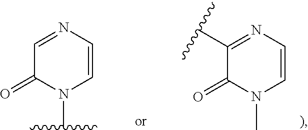

and imidazolone (e.g.,

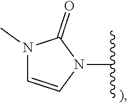 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, said heteroatoms selected from O, N, S (inclusive of oxidized forms such as:

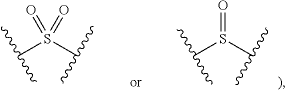

and P (inclusive of oxidized forms such as

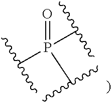 [SMILES: *P(*)(*)=O]

(e.g., N, O, and S (inclusive of oxidized forms such as:

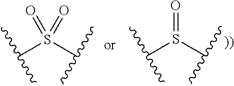

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl.

Heterocyclyl may include multiple fused and bridged rings. Non-limiting examples of fused/bridged heteorocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrolyl, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

A nitrogen-containing heterocyclyl as used herein refers to a heterocyclyl having 1-2 ring nitrogen atoms and 0-2 additional ring heteroatoms selected from the group consisting of O and S (inclusive of oxidized such as:

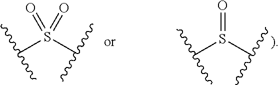

The nitrogen-containing heterocyclyl can be monocyclic, bicyclic, or polycyclic as defined elsewhere herein. Examples of monocyclic nitrogen-containing heterocyclyl include azetidinyl, pyrrolidinyl, piperidinyl, piperazinyl, morpholinyl, and the like. Examples of bicyclic nitrogen-containing heterocyclyl include 7-azaspiro[3.5]nonyl, 1,7-diazaspiro[4.5]decyl, 3-oxa-7,9-diazabicyclo[3.3.1]nonanyl, 2,6-diazaspiro[3.3]heptanyl, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge (e.g.,

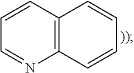

(ii) a single ring atom (spiro-fused ring systems) (e.g.,

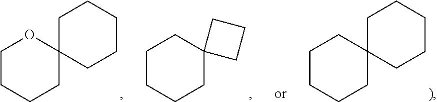

or (iii) a contiguous array of ring atoms (bridged ring systems having all bridge lengths >0) (e.g.,

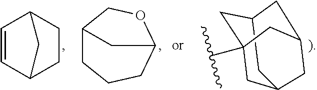

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

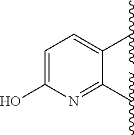 [SMILES: *c1ccc(O)nc1*]

encompasses the tautomeric form containing the moiety:

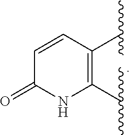 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

### Methods of Treatment

### Indications

Provided herein are methods for inducing degradation of a BCL6 protein. For example, provided herein are compounds capable of inducing degradation of a BCL6 protein useful for treating or preventing cancers. Exemplary compounds that bind to BCL6 are described in, e.g., Cerchietti, Leandro C., et al. Cancer Cell 17.4 (2010): 400-411; Cardenas, Mariano G., et al. The Journal of Clinical Investigation 126 (9) (2016): 3351-3362; Kerres, Nina, et al., Cell Reports 20.12 (2017): 2860-2875; Yasui, Takeshi, et al., Bioorganic & Medicinal Chemistry 25.17 (2017): 4876-4886; Kamada, Yusuke, et al., Journal of Medicinal Chemistry 60.10 (2017): 4358-4368; McCoull, William, et al., ACS Chemical Biology 13.11 (2018): 3131-3141; Guo, Weikai, et al. Journal of Medicinal Chemistry 63.2 (2020): 676-695; Teng, Mingxing, et al., ACS Medicinal Chemistry Letters 11.6 (2020): 1269-1273; Pearce, Andrew C., et al., Journal of Biological Chemistry 297.2 (2021); Ding, Shu, Yu Rao, and Qianjin Lu, Cellular & Molecular Immunology (2022): 1-3; Xing, Y. et al., Cancer Letters (2022), doi: 10.1016/j.canlet.2021.12.035; Huckvale, R. et al., Journal of Medicinal Chemistry (2022), doi: 10.1021/acs.jmedchem.1c02175; Davis, O. et al., Journal of Medicinal Chemistry (2022), doi: 10.1021/acs.jmedchem.1c02174; International Publication Nos. WO 2008/066887; WO 2010/008436; WO 2014/204859; WO 2018/215798; WO 2018/215801; WO 2018/219281; WO 2019/119138; WO 2019/119144; WO 2019/119145; WO 2019/153080; WO 2019/197842; WO 2020/104820; WO 2021/074620; WO 2021/077010, and WO 2022/221673.

Potency of degradation by a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein can be determined by DC₅₀ value. As used herein, DC₅₀ refers to the concentration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II) that results in a 50% decrease in the concentration of a protein (e.g., BCL6 protein) in a cell compared to the concentration of the protein before the cell is contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or compared to the concentration of the protein in a cell not contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II). A compound with a lower DC₅₀ value, as determined under substantially similar conditions, is a more potent inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

Potency of degradation by a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein can be determined by EC₅₀ value. As used herein, EC₅₀ refers to the concentration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II) that results in a 50% decrease in the concentration of a protein (e.g., BCL6 protein) relative to the trough concentration of the protein in a cell, when compared to the concentration of the protein before the cell is contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or compared to the concentration of the protein in a cell not contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II). A compound with a lower EC₅₀ value, as determined under substantially similar conditions, is a more potent inducer of degradation relative to a compound with a higher EC₅₀ value. In some embodiments, a EC₅₀ value can be determined (e.g., using HiBiT detection) in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

Potency of degradation by a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein can be determined by a Yₘᵢₙ value. As used herein, Yₘᵢₙ refers to the ratio of trough concentration of a protein (e.g., BCL6 protein) in a cell compared to the concentration of the protein before the cell is contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or compared to the concentration of the protein in a cell not contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), expressed as a percentage. As used herein, Dₘₐₓ is 1−Yₘᵢₙ. Yₘᵢₙ can be measured by a HiBiT assay as described in Example B1. A compound with a lower Yₘᵢₙ value, as determined under substantially similar conditions, is a more potent inducer of degradation relative to a compound with a higher Yₘᵢₙ value. In some embodiments, a Yₘᵢₙ value can determined (e.g., using HiBiT detection), in vitro or in vivo (e.g., in tumor cells (e.g., cell lines such as A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2; see also those disclosed in, e.g., Cardenas, Mariano G., et al. Clinical Cancer Research 23.4 (2017): 885-893 and International Publication Nos. WO 2021/080950, WO 2021/077010, and WO 2022/221673) expressing a BCL6 protein)). In some embodiments, a cell line that is not dependent on BCL6 and/or that does not have significant expression of BCL6 can be used as a control (e.g., Toledo, H929, MM.1S, or OPM2).

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Y min of less than 70% (e.g., less than 50% or less than 30%) in a HiBiT based degradation assay (e.g., an assay as described in Example B1). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 50% (e.g., less than 30%) in a HiBiT based degradation assay. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 30% in a HiBiT based degradation assay.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Y min of less than 70% (e.g., less than 50% or less than 30%) in the assay described in Example B1. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 50% (e.g., less than 30%) in the assay described in Example B1. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of less than 30% in the assay described in Example B1.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 70% (e.g., about 0% to about 50%, about 30% to about 50%, or about 0% to about 30%) in a HiBiT based degradation assay (e.g., an assay as described in Example B1). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 50% (e.g., about 30% to about 50% or about 0% to about 30%) in a HiBiT based degradation assay. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 30% in a HiBiT based degradation assay.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 70% (e.g., about 0% to about 50%, about 30% to about 50%, or about 0% to about 30%) in the assay described in Example B1. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 50% (e.g., about 30% to about 50% or about 0% to about 30%) in the assay described in Example B1. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, exhibits a Yₘᵢₙ of about 0% to about 30% in the assay described in Example B1.

It will be understood that the effect of protein degradation typically increases over time, though the appearance of degradation (e.g., as expressed by the percentage degradation compared to a control, or the parameters Yₘᵢₙ, DC₅₀, and/or Dₘₐₓ) is affected by the resynthesis rate of the protein. It is common in the art to examine degradation after a specified period of time, such as 6 hours, 12 hours, 18 hours, 1 day, 2 days, 3 days, 5 days, 10 days, or more. For example, degradation can be expressed as the percent degradation after 24 hours.

Exemplary assays for validating the degradation-inducing mechanism of a compound as provided herein are known in the art and are described, for example, in Wu, et al. Nature Structural & Molecular Biology 27.7 (2020): 605-614.

Degradation assays can be used to quantify both on- and off-target degradation-inducing effects of compounds, such as those provided herein. Exemplary assays include, quantitative immunoblotting, other immunoassays (e.g., MesoScale Discovery (MSD) immunoassays), homogenous time resolved florescence (HTRF), and HiBiT. In some embodiments, cells can be contacted with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL6 protein, and the amount of fluorescence observed when the complementary LgBiT peptide is added can be compared between cells treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and a control (e.g., a DMSO-treated control). See, for instance, Example B1. In some embodiments, off-target degradation inducing effects can be assessed for the proteins Eukaryotic peptide chain release factor GTP-binding subunit ERF3A (GSPT1), Ikaros (IKZF1), Helios (IKZF2), Aiolos (IKZF3), and/or casein kinase I isoform alpha (CK1α). See also, e.g., International Publication Nos. WO 2018/215798; WO 2018/215801; WO 2020/104820; McCoull, William, et al., ACS Chemical Biology 13.11 (2018): 3131-3141. Bellenie, Benjamin R., et al., Journal of Medicinal Chemistry 63.8 (2020): 4047-4068; Lloyd, Matthew G., et al., Journal of Medicinal Chemistry 64.23 (2021): 17079-17097.

Binding affinity of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein to BCL6 can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a K_{D} value can be determined by surface plasmon resonance (SPR) or biolayer interferometry; see, e.g., Guo, Weikai, et al., Journal of Medicinal Chemistry 63.2 (2020): 676-695; Lloyd, Matthew G., et al., Journal of Medicinal Chemistry 64.23 (2021): 17079-17097 and International Publication Nos. WO 2019/153080; WO 2019/119144; and WO 2019/119145.

The ability of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as provided herein to inhibit BCL6 can be determined using an IC₅₀ value. A compound with a lower IC₅₀ value, as determined under substantially similar conditions, is a more potent inhibitor relative to a compound with a higher IC₅₀ value. For example, an IC₅₀ value can be calculated using a FRET (e.g., Homogeneous Time Resolved Fluorescence (HTRF)) assay, where a tagged (e.g., His-tagged) BCL6 protein and tagged (e.g., fluorophore-tagged (e.g., Alexa-Fluor633)) corepressor peptide (e.g., BCOR) are incubated in the presence of compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), and subsequently, the FRET ratio (relative to appropriate controls) can be measured using an appropriate FRET pair (e.g., with an antibody that recognizes the tagged BCL6 protein (e.g., anti-His-Terbium cryptate)). See, e.g., International Publication Nos. WO 2018/108704; WO 2018/215798; WO 2019/197842; WO 2020/104820; WO 2021/074620. As another example, an IC₅₀ value can be calculated using an enzyme-linked immunosorbent assay (ELISA) using a tagged (e.g., biotinylated) corepressor peptide (e.g., BCOR) immobilized on a substrate and a tagged (e.g., FLAG-tagged) BCL6 (e.g., a domain, such as the BTB domain, thereof), where compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II) can be used to prevent the interaction between the corepressor peptide and BCL6, and the interaction between the corepressor peptide and BCL6 can be measured using an antibody to the BCL6 construct (e.g., anti-FLAG antibody). See, e.g., Kamada, Yusuke, et al., Journal of Medicinal Chemistry 60.10 (2017): 4358-4368. As yet another example, an IC₅₀ value can be calculated using a florescence polarization assay with a fluorescently-tagged corepressor peptide (e.g., SMRT) where compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II) can be used to prevent the interaction between the corepressor peptide and BCL6. See, e.g., International Publication No. WO 2019/119144. As another example, a cellular IC₅₀ value can be calculated using a BRET (Bioluminescence Resonance Energy Transfer) assay, where vectors encoding BCL6 and a corepressor peptide (e.g., SMRT), complementarily fused with NanoLuc or HaloTag, can be inserted into cells. The cells can be treated with compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, to determine the effect of the compounds on inhibiting the BCL6-corepressor interaction. See, e.g., International Publication Nos. WO 2018/215798 and WO 2019/197842. In another example, an IC₅₀ value for the inhibition of BCL6 repressor function can be calculated using a luciferase assay, where cells are engineered to express luciferase under the control of one or more BCL6 repressor sites, and the cells can be incubated with compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, to determine the effect of the compounds on the function of the BCL6 repressor. See, e.g., International Publication Nos. WO 2019/119144; WO 2019/119145; WO 2019/153080.

Another exemplary way of evaluating the effect of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is to measure the induction (e.g., fold induction) of genes that are typically repressed by BCL6 (e.g., p53, ATR, CXCR3, CD69, and CDKN1A) using a method such as RT-PCR. See, e.g., Guo, Weikai, et al., Journal of Medicinal Chemistry 63.2 (2020): 676-695.

An exemplary assay for determining the potency of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, includes measuring the effect of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, on cell proliferation and/or viability. Cell proliferation assays can be performed in a number of formats, including 2D and 3D. Similarly, a cell proliferation assay can be performed with any appropriate cell line, including, for example, A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2. In some embodiments, a cell line that is not dependent on BCL6 and/or that does not have significant expression of BCL6 can be used as a control (e.g., Toledo, H929, MM.1S, or OPM2). As an illustrative example, a 3D cell proliferation assay can include growing cells in a 3D medium, contacting the cells with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, measuring the cellular proliferation using an appropriate reagent (e.g., CELLTITERGLO® 3D), and then comparing the signal from an experiment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to the signal from a control experiment (e.g., lacking the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof). As another illustrative example, a 2D cell proliferation assay can include plating cells onto a growth surface, optionally letting the cells grow for a period of time, contacting the cells with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, measuring the cellular proliferation using an appropriate reagent (e.g., CELLTITERGLO®), and then comparing the signal from an experiment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to the signal from a control experiment (e.g., lacking a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts, ATPlite assays, and other methods are known in the art. See, for instance, Example B2. See also, e.g., Guo, Weikai, et al., Journal of Medicinal Chemistry 63.2 (2020): 676-695; McCoull, William, et al. ACS Chemical Biology 13.11 (2018): 3131-3141; Lloyd, Matthew G., et al. Journal of Medicinal Chemistry 64.23 (2021): 17079-17097; Bellenie, Benjamin R., et al. Journal of Medicinal Chemistry 63.8 (2020): 4047-4068; and International Publication Nos. WO 2018/215798; WO 2018/215801; WO 2018/219281; WO 2019/119145; WO 2019/153080; and WO 2020/104820.

A cell viability assay can be used to measure the effect of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, on cell death. For example, cells expressing BCL6 protein (e.g., A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2 cells) can be incubated with various concentrations of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, then exposed to a detection reagent (e.g., using a CELLTITER-GLO® Cell Viability Assay kit) to determine cell viability. In some embodiments, the effect on cell viability can be compared to a cell line that is not dependent on BCL6 and/or that does not have significant expression of BCL6 can be used as a control (e.g., Toledo, H929, MM.1S, or OPM2).

A cell viability assay can be used to measure the effect of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, on cell death in combination with an additional therapeutic agent. For example, cells expressing BCL6 protein (e.g., A3/KAW, A4/FUK, DB, DOHH2, Farage, HT, Karpas 422, KML1, MHHPREB1, NUDHL1, OCI-Ly1, OCI-Ly3, OCI-Ly7, OCI-Ly18, OCI-Ly19, Pfeiffer, RI1, RL, SU-DHL-4, SU-DHL-5, SU-DHL-6, SU-DHL-8, SU-DHL-10, VAL, or WSU-DLCL2 cells) can be incubated (e.g., for 72 hours or for 120 hours) in a 7×7 dose matrix at various concentrations of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and an additional therapeutic agent (e.g., any of the additional therapeutic agents described herein) (e.g., half-log diluted from 316 to 1 nM), then exposed to a detection reagent (e.g., using a CELLTITER-GLO® Cell Viability Assay kit) to determine cell viability. The combination activity can be assessed by the Bliss independence model: negative values as indication of antagonism, positive as synergy, and a value of zero as additive activity. Bliss scores in the dose matrix can be added up to give a “Bliss sum” value to reflect the overall synergy activity of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent in each cell line. In some embodiments, a cell line that is not dependent on BCL6 and/or that does not have significant expression of BCL6 can be used as a control (e.g., Toledo, H929, MM.1S, or OPM2).

As another example, the potency and/or efficacy of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be evaluated in an animal model, for example, a cell line-derived (CDX) xenograft model (e.g., using an established cancer cell line such as DB, DoHH2, OCI-Ly1, OCI-Ly7, RL, Pfeiffer, SU-DHL-5, SU-DHL-6, WSU-DLCL2, REH, BALL-1, RS4;11, SEMK2, KOPN8, NALM-6, KASUMI-2, RCH-ACV, SUP-B15, BV-173, TOM-1, NALM-20, NALM-21, MUTZ-5, or MHH-CALL-4 (e.g., OCI-Ly1, OCI-Ly7, SU-DHL-5, SU-DHL-6, WSU-DLCL2, DB, RL, Pfeiffer, or DoHH2)), a genetically engineered mouse model (GEMM) or a patient-derived xenograft (PDX) model. For example, a PDX model can be run in immunodeficient mice (e.g., athymic nude, outbred homozygous (e.g., Crl:NU(NCr)-Foxn1^{nu}) or Fox Chase SCID (CB17/Icr-Prkdc^{scid}/IcrIcoCrl), mice). The mice can be female, 6-12 weeks old at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, (e.g., via IP or PO administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study the Best Response can be calculated for each treatment arm. Best Response is defined as the minimum value of ΔVolumeₜ for t≥10 days. Best Responses between the control arm(s) and the treatment arm(s) can be compared to determine if the treatment(s) work better than the control(s). In some embodiments, tumor samples can also be collected at the end of each study and relevant proteins (e.g., BCL6) can be measured to determine if the treatment has a better protein modulation profile compared to a control. In some embodiments, tumor samples and/or blood samples can also be collected at the end of each study and analyzed for altered gene expression activity (e.g., altered ARID3A, ARID3B, ATR, B2M, BANK1, BATF, BCL11A, BCL2, BCL2A1, BLIMP1, BM11, CASP8, CCND1, CCND2, CCR6, CCR7, CD38, CD44, CD69, CDKN1A, CDKN1B, CFLAR/FLIP, CHEK1, CXCR4, CXCR5, DR5, EBI2, ETV6, FCMR, FGD4, ID2, IFITM1, IFITM2, IFNAR2, IFNGR1, IL10, IL10RB, IL7R, CXCL10, IRF1, IRF4, IRF7, IRF9, JAK3, JARID2, JUN, KLF2, LITAF, MCL1, MIP-1a, MYC, MYD88, NFKBIE, NOTCH2, PDL1, PIM1, PPP3R1, PRDM1, PTEN, SIPR1, SHP1, STATI, STAT3, STAT5A, TLR1, TLR4, TLR7, TLR9, TNF-R2, TOX, TP53, ZEB2, and/or ZNF608 expression levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 1, 3 and/or 5, with collections at 6, 12 and/or 24 hours post-dosing, and relevant proteins can be measured in the tumor samples and pharmacokinetic studies can be performed on the blood samples or a portion thereof (e.g., plasma). In some embodiments, the PDX is a model of B-ALL (e.g., Philadelphia chromosome positive B-ALL, Philadelphia chromosome negative B-ALL, or B-ALL with an MLL-Af4 fusion, an MLL-Af6 fusion, an MLL-Af9 fusion, an MLL-ENL fusion, or an MLL-PTD fusion), DLBCL, FL, MCL, Burkitt lymphoma, or peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)). See, e.g., Guo, Weikai, et al., Journal of Medicinal Chemistry 63.2 (2020): 676-695.

In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, exhibit activity in a model of an autoimmune disease. Exemplary assays can be found in, for example, International Publication Nos. WO 2020/014599 and WO 2021/074620.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof, can be assessed for its ability to modulate (e.g., decrease) IgG antibody production and/or modulate (e.g., decrease) germinal center formation in an animal (e.g., mouse) following challenge with a T cell-dependent antigen (e.g., keyhole limpet haemocyanin (KLH)). For example, KLH can be administered to mice (e.g., C57BL/6 mice), followed by administration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time (e.g., 14 days). Following sacrifice, serum samples from the mice can be analyzed for IgG specific for KLH, for example, by ELISA. Similarly, germinal centers can be detected using immunohistological staining using, e.g., peanut agglutinin (PNA). See, e.g., Example 1 of WO 2020/014599.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be assessed for its ability to modulate (e.g., decrease) the number of germinal center B cells in an animal (e.g., mouse) following immunization with an antigen. For example, animals (e.g., mice) can be immunized with Complete Freund's Adjuvant (CFA), and after a period of time (e.g., 8 days), the animals can be sacrificed and the spleens harvested. The spleens can be processed into suspension and cultured in the presence of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof. The cells can then be analyzed, for example, for the number of germinal center B cells (e.g., by flow cytometry using the lineage markers GL7 and CD95). See, e.g., WO 2021/074620.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof, can be assessed for its ability to improve one or more symptoms, biomarkers, or other signs of an autoimmune disease in an animal model of the autoimmune disease.

Experimental autoimmune encephalitis (EAE) can be used as an animal model of inflammatory diseases of the CNS, including multiple sclerosis (MS) and neuromyelitis optica. In some cases, EAE can be induced in animals (e.g., mice), for example, via immunization with recombinant human myelin oligodendrocyte glycoprotein (MOG) or a fragment thereof (e.g., MOG₁₋₁₂₅), myelin basic protein, and/or proteolipid protein. Following induction of EAE, the animals can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated by, for example, EAE severity, B cell depletion, or both. See, e.g., Constantinescu, Cris S., et al. British Journal of Pharmacology 164.4 (2011): 1079-1106; Monson, Nancy L., et al. PloS One 6.2 (2011): e17103.

In some embodiments, the autoimmune disease is anti-synthetase syndrome. For example, a model of anti-synthetase syndrome, can be induced in susceptible mice (e.g., C57BL/6, B6.G7, and/or NOD.Idd3/5) by immunization with histidyl-tRNA synthetase (e.g., murine histidyl-tRNA synthetase or human histidyl-tRNA synthetase), or a fragment thereof. Treatment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, may begin at immunization and continue for a period of time (e.g., short term (e.g., 10-14 days) or long term (e.g., 8-16 weeks)), then the mice can be sacrificed. The mice can be evaluated, for example, for generation of histidyl-tRNA synthetase-specific antibody (e.g., via immunoprecipitation, ELISA, and/or flow cytometry), tissue (e.g., lung and/or muscle) inflammation (e.g., by pathologist review), or a combination thereof. See e.g., Katsumata, Yasuhiro, et al. Journal of Autoimmunity 29.2-3 (2007): 174-186; Katsumata, Yasuhiro, et al. Journal of Autoimmunity 29.2-3 (2007): 174-186; Ascherman, Dana P. Current Rheumatology Reports 17 (2015): 1-7; and Konishi, Risa, Yuki Ichimura, and Naoko Okiyama. Immunological Medicine 46.1 (2023): 9-14.

In some embodiments, the autoimmune disease is arthritis (e.g., rheumatoid arthritis or inflammatory arthritis). For example, a mouse model of rheumatoid arthritis, collagen induced arthritis (CIA), can be induced in susceptible mice (e.g., DBA/1 or HLA-DR) by immunization with type II collagen (CII). Treatment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, may begin at immunization and continue for a period of time (e.g., 6 weeks). The mice can be evaluated, for example, for clinical scores (e.g., inflammation of an arthritic limb, foot thickness, paw volume (e.g., using a plethysmometer), or a combination thereof), generation of CII-specific antibody, B cell depletion, or a combination thereof. See e.g., Example 2 of WO 2020/014599; Brand, David D., et al. Nature Protocols 2.5 (2007): 1269-1275. Additional animal models of arthritis (e.g., inflammatory arthritis) are known in the art, such as K/B×N mice. In some embodiments, such mice can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and evaluated, for example, in similar ways to CII-immunized mice, or additionally, titering for autoantibodies, such as those against glucose-6-phosphate isomerase. See, e.g., Huang, Haochu, Christophe Benoist, and Diane Mathis. Proceedings of the National Academy of Sciences 107.10 (2010): 4658-4663; and Pigott, Elizabeth, and Laura Mandik-Nayak. Arthritis & Rheumatism 64.7 (2012): 2169-2178.

In some embodiments, the autoimmune disease is graft-versus-host disease (e.g., chronic graft-versus-host disease). In some cases, an animal model of graft-versus-host disease can be in animals (e.g., mice) conditioned with high-dose cyclophosphamide and lethal total-body irradiation (TBI) rescued with bone marrow optionally including allogeneic splenocytes or purified T cells. Administration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time can follow. The animals can be evaluated by, for example, pulmonary function tests, by immunohistochemistry to evaluate the presence of autoantibodies, or by examining sections of the spleen for germinal centers, including for example, examining for B cell depletion (e.g., germinal center B cell depletion). See, e.g., Srinivasan, Mathangi, et al. Blood, The Journal of the American Society of Hematology 119.6 (2012): 1570-1580; and Paz, Katelyn, et al. Blood, The Journal of the American Society of Hematology 133.1 (2019): 94-99. See also, e.g., Dubovsky, Jason A., et al. The Journal of Clinical Investigation 124.11 (2014): 4867-4876.

In some embodiments, the autoimmune disease is IgG4-related disease (IgG4-RD). In some cases, an animal model of IgG4-RD can be generated by injecting mice with IgGs (e.g., IgG1 and/or IgG4) derived from human IgG4-RD patients. Such mice can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated by measuring, for example, pancreatic and/or salivary tissue damage, B cell depletion, or both. See, e.g., Shiokawa, Masahiro, et al. Gut 65.8 (2016): 1322-1332.

In some embodiments, the autoimmune disease is lupus (e.g., lupus erythematosus). In some cases, an animal model of lupus is MRL/Ipr mice, which can display high expression of Tfh-associated molecules such as ICOS, PD-1, BCL-6, and IL-21 and produce autoantibodies to nuclear components, develop nephritis, arthritis, and skin lesions. Such mice can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated by, for example, measuring B cell depletion (e.g., germinal center B cell depletion), identifying the presence and/or severity of glomerulonephritis, autoantibody titers (e.g., anti-RNA antibody titers, anti-nuclear antibody titers, and/or anti-dsDNA antibody titers), IL-21 expression, and/or amount of activated CD4+ T cells. See, e.g., Ahuja, Anupama, et al. The Journal of Immunology 179.5 (2007): 3351-3361; Shen, Chunxiu, et al. Journal of Cellular and Molecular Medicine 25.17 (2021): 8329-8337; and Marinov, Anthony D., et al. Arthritis & Rheumatology 73.5 (2021): 826-836. Additional animal models of lupus are known in the art, such as NZB/W mice, which can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and evaluated in similar ways to the MRL/Ipr mice. See, e.g., Wang, Wensheng, et al. The Journal of Immunology 192.7 (2014): 3011-3020; and Kansal, Rita, et al. Science Translational Medicine 11.482 (2019): eaav1648.

In some embodiments, the autoimmune disease is myasthenia gravis (e.g., muscle-specific tyrosine kinase (MuSK) positive myasthenia gravis). For example, an animal model (e.g., a rat model, a mouse model, or a rabbit model) of myasthenia gravis can be generated by immunizing the animal with acetylcholine receptor from Torpedo (e.g., Torpedo californica) or Electrophorus (e.g., Electrophorus electricus) electric organs, or recombinant acetylcholine receptor protein or fragments thereof. Treatment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can begin, for example, approximately 4 weeks after immunization, but can sometimes begin earlier or later. The rodents can be evaluated, for example, for clinical scores (e.g., grasping and/or lifting of a weight, while looking for tremor, hunched posture, muscle strength, and signs of fatigue), body weight, compound muscle action potential (e.g., using electromyography), anti-acetylcholine receptor antibodies, B cell depletion, or a combination thereof. See, e.g., Mori, Shuuichi, et al. The American Journal of Pathology 180.2 (2012): 798-810; Xin, Ning, et al. Molecular and Cellular Neuroscience 58 (2014): 85-94; and Losen, Mario, et al. Experimental Neurology 270 (2015): 18-28.

In some embodiments, the autoimmune disease is multiple sclerosis (MS). In some embodiments, the MS is clinically isolated syndrome (CIS), relapsing-remitting MS (RRMS), primary progressive MS (PPMS), or secondary progressive MS (SPMS). In some embodiments, animal models of MS can be based on EAE, for example, relapsing-remitting EAE in SJL/J mice (e.g., via immunization with proteolipid protein (PLP) 139-151), chronic EAE in C57BL/6J mice (e.g., via immunization with MOG₃₅₋₅₅), or EAE in transgenic mice (e.g., via a T cell clone expressing Vα and Vβ chains reacting specifically to MOG₃₅₋₅₅, or a B cell heavy chain knock-in mouse). In some embodiments, the animal (e.g., mouse) model is generated via infection with a picornavirus, such as Theiler's murine encephalitis virus. In some embodiments, the animal (e.g., mouse) model is generated by feeding C57BL/6 mice with cuprizone (e.g., 0.2% for 6 weeks). In some embodiments, the animal (e.g., mouse) model is generated via lysolecithin injection (e.g., in SJL/J mice). Treatment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be followed, for example, by analysis of the animals for levels of myelin-specific T cells, B cell depletion, reduction of inflammatory lesions, axonal degeneration, protection or reversal of cuprizone or lysolecithin-induced demyelination, or a combination thereof. See, e.g., Procaccini, Claudio, et al. European Journal of Pharmacology 759 (2015): 182-191.

In some embodiments, the autoimmune disease is neuromyelitis optica (NMO). In some cases, animal models of NMO include administration of antibodies against astrocyte water channel aquaporin-4 (AQP4), called AQP4-IgG or NMO-IgG, to various CNS tissues, such as the brain. In some embodiments, the administration of the antibodies is to an animal with EAE. The AQP4-IgG may be recombinant or derived from human NMO patients. Treatment with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salt thereof, can be followed, for example, by analysis of the sacrificed animal for slowing or reversal of astrocyte demyelination, number of macrophages, number of activated microglia, number of activated neutrophils, B cell depletion, or a combination thereof. See, e.g., Bennett, Jeffrey L., et al. Annals of Neurology: Official Journal of the American Neurological Association and the Child Neurology Society 66.5 (2009): 617-629; Saini, Harleen, et al. BMC Neurology 13.1 (2013): 1-9; Oji, Satoru, et al. PloS One 11.3 (2016): e0151244; Peschl, Patrick, et al. Journal of Neuroinflammation 14.1 (2017): 1-14; and Duan, Tianjiao, and Alan S. Verkman. Brain Pathology 30.1 (2020): 13-25.

In some embodiments, the autoimmune disease is pemphigus (e.g., pemphigus vulgaris). In some cases, an animal (e.g., mouse) model of pemphigus can be generated using adoptive transfer of peripheral lymphocytes from Dsg3 knockout animals to immune-deficient but desmoglein 3-expressing recipient mice to create an artificial immune state in the recipient animals. In some cases, an animal (e.g., mouse) model of pemphigus can be generated using an animal with a MHC class II-null background that expresses the pemphigus-associated HLA-DRB1*0402 allele, which after immunization with recombinant human DSG3, produce anti-human DSG3 antibodies. Treatment of these models with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be followed, for example, by analysis of the B cell repertoires of the animals for anti-desmoglein B-cell clones, B cell depletion, or both. See, e.g., Kasperkiewicz, Michael, et al. Nature Reviews Disease Primers 3.1 (2017): 1-18.

In some embodiments, the autoimmune disease is Sjogren's syndrome. In some cases, an animal model of Sjogren's syndrome is a NOD mouse, which sometimes has further genetic manipulation or crosses (e.g., NOD.B10.H2^{b} or C57BL/6.NOTDc3.NODclt) to recapitulate the symptoms of the syndrome. Such mice can be treated with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. The animals can be evaluated, for example, for salivary and lacrimal gland secretory flow rates, salivary protein content, autoantibodies to exocrine gland proteins (e.g., anti-Sjogren's syndrome A (SSA) antibodies, anti-Sjogren's syndrome B (SSB) antibodies, and/or anti-muscarinic acetylcholine 3 receptor (M3R) antibodies), B cell depletion, or a combination thereof. Robinson, Christopher P., et al. Arthritis & Rheumatism 41.1 (1998): 150-156; Cha, Seunghee, et al. “Arthritis & Rheumatism 46.5 (2002): 1390-1398; and Ohno, Seiji, et al. Autoimmunity 45.7 (2012): 540-546. Additional animal models of Sjogren's syndrome are known in the art and include, for example, NFS/sld mice, IQI/Jic mice, Aly/aly mice, and mice immunized with M3R peptides, which can be evaluated, in some cases, for the same parameters as NOD mice. See, e.g., Iizuka, Mana, et al. Journal of Autoimmunity 35.4 (2010): 383-389; and Park, Young-Seok, Adrienne E Gauna, and Seunghee Cha. Current Pharmaceutical Design 21.18 (2015): 2350-2364.

The pharmacokinetic parameters of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be evaluated in an animal model, for instance, a mouse model, a rat model, a dog model, or a nonhuman primate (e.g., cynomolgus monkey) model. Pharmacokinetics (PK) studies can be conducted in an animal model (e.g., male CD-1 mice) by two exemplary delivery routes: intravenous (IV) injection and oral (PO), for example, oral gavage. Animals in the IV group (e.g., n=3) are allowed free access to food and water; animals in the PO group are allowed free access to food or are fasted for 6-8 hours prior to dosing. A compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be formulated in solution for the IV route and solution or suspension for the PO route. On the day of the experiment, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be administered via vein injection (e.g., at 1 mg/kg) for IV route or via oral gavage (e.g., at 3 to 90 mg/kg, or 3 to 10 mg/kg, such as 10 mg/kg) for PO route. In some cases, the animals can be orally pre-dosed with a cytochrome P450 inhibitor (e.g., 1-aminobenzotriazole) prior to (e.g., 16 hours prior to) dosing the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. Blood samples can be collected via serial bleeding (e.g., at 8 timepoints from 0.83 to 24 hours post dose). At each timepoint, blood (e.g., about 30 μL to about 125 μL, or about 75 μL to about 125 μL) can be collected (e.g., via the saphenous vein) in a tube containing an anti-coagulant (e.g., K₂EDTA). Blood samples can be put on wet ice and centrifuged (e.g., at 2000×g for 4-10 minutes) to obtain plasma samples. Plasma samples can be diluted (e.g., with an equal volume of pH 3.0 phosphate buffer or with an equal volume of pH 5.0 sodium citrate) and submitted to LC-MS/MS for sample analysis. Pharmacokinetics parameters, including clearance (CL), volume of distribution (V_{d}), maximum plasma concentration (Cₘₐₓ), time of maximum plasma concentration (tₘₐₓ), half-life (t_{1/2}), area under the curve (AUC), and oral bioavailability (% F) can be calculated using a non-compartmental model.

In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 4%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 10%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 20%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 30%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 40%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 60%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is at least 80%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 4% to about 90% (e.g., about 4% to about 80%, about 4% to about 60%, about 4% to about 40%, about 4% to about 20%, about 4% to about 10%, about 20% to about 40%, about 40% to about 60%, about 60% to about 80%, or about 70% to about 90%). In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 4% to about 20%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 20% to about 40%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 40% to about 60%. In some embodiments, the % F for a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is about 50% to about 80%.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not a substrate of a human cytochrome P450 enzyme. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not a substrate of a human cytochrome P450 enzyme where ≥25% of clearance is attributed to that enzyme. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not an inhibitor and/or an inducer of one or more human cytochrome P450 enzymes. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not an inhibitor and/or an inducer of one or more human cytochrome P450 enzymes, where the IC₅₀ and/or EC₅₀ for the one or more human cytochrome P450 enzymes, respectively, is at a concentration to be significantly greater than the estimated free fraction concentration of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, at a clinically relevant dose.

Exemplary human cytochrome P450 enzymes include those in the CYP1, CYP2, and CYP3 families. For example, any one of CYP1A1, CYP1A2, CYP1B1, CYP2A6, CYP2B6, CYP2C8, CYP2C9, CYP2C18, CYP2C19, CYP2D6, CYP2J2, CYP2S1, CYP2E1, CYP3A4, and CYP3A5. In some embodiments, no single cytochrome P450 enzyme is responsible for greater than or equal to 25% of the elimination of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. Cytochrome P450 inhibition and/or inducing activity can be determined using any appropriate assay, such as those described in the guidance document “In Vitro Drug Interaction Studies Cytochrome P450 Enzyme- and Transporter-Mediated Drug Interactions” provided by the U.S. F.D.A. in January 2020. For example, evaluation of cytochrome P450 inhibition can be performed in in vitro studies, in both a reversible and time-dependent manner. In an in vitro inhibition study, the ratio of intrinsic clearance values of a probe substrate for an enzymatic pathway in the absence and in the presence of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be calculated; this ratio is referred to as R₁ for reversible inhibition, where R₁=1+(I_{max,u}/K_{i,u}), and I_{max,u} is the maximal unbound plasma concentration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and K_{i,u} is the unbound inhibition constant determined in vivo. Specifically, for CYP3A, R_{1,gut} can be calculated where R_{1,gut}=1+(I_{gut}+K_{i,u}), and where I_{gut} is the intestinal luminal concentration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, calculated as the dose/250 mL. The time-dependent inhibition ratio R₂ can similarly be calculated, where R₂=(k_{obs}+k_{deg})/k_{deg}, and k_{obs} is the observed (apparent first order) inactivation rate of the affected cytochrome P450 calculated by k_{obs}=(k_{inact}*50*I_{max,u})/(K_{I,u}+50*I_{max,u}), k_{deg} is the apparent first-order degradation rate constant of the affected cytochrome P450, K_{I,u} is the unbound concentration of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, causing half maximal inactivation, and k_{inact} is the maximal inactivation rate constant. If R₁≥1.02, R₂≥1.25, and/or R_{1,gut}≥11, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, may be an inhibitor of a cytochrome P450, and the drug-drug interaction (DDI) potential can be further investigated using mechanistic models and/or conducting a clinical DDI study with a sensitive index substrate. For example, evaluation of cytochrome P450 induction can be performed via the fold-change method, wherein the fold-change in cytochrome P450 enzyme mRNA levels when incubated with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, by using a cutoff determined from known positive and negative controls to calibrate the system. For example, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is interpreted as an inducer if: (1) it increased mRNA expression of a cytochrome P450 enzyme in a concentration-dependent manner; and (2) the fold change of cytochrome P450 mRNA expression relative to the vehicle control is ≥2-fold at the expected hepatic concentrations of the drug. As another example, evaluation of cytochrome P450 induction can be performed by a correlation method, wherein correlation methods are used to predict the magnitude of a clinical induction effect (e.g., AUC ratio of an index substrate in the presence and absence of inducers) of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, according to a calibration curve of relative induction scores (RIS) or I_{max,u}/EC₅₀ for a set of known inducers of the same cytochrome P450. If the predicted magnitude is more than a predefined cut-off (e.g., AUC ratio≤0.8), a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is considered to have induction potential in vivo.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is not a hERG inhibitor. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 60 nM (e.g., greater than 100 nM, 300 nM, 500 nM, 1 μM, 3 μM, 5 μM, 10 μM, 20 μM, or 30 μM). For example, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 500 nM (e.g., 1 μM, 3 μM, 5 μM, 10 μM, 20 μM, or 30 μM). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 1 μM (e.g., greater than 3 μM, 5 μM, 10 μM, 20 μM, or 30 μM). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 10 μM (e.g., greater than 20 μM or 30 μM). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, inhibits hERG with an IC₅₀ of greater than 30 μM.

Heterobifunctional degraders can, in some cases, induce the degradation of off-target proteins. For heterobifunctional degraders that utilize CRBN, common off-target proteins that can be degraded include GSPT1, IKZF1, IKZF2, IKZF3, and/or CK1α. This degradation is generally believed to be due to the E3 binding moiety of the heterobifunctional degrader facilitating ternary complex formation between the off-target protein and CRBN. GSPT1 is a translation termination factor, and CK1α is a kinase that is involved in many key cellular processes including cell cycle progression and chromosome segregation; these are both commonly essential genes, so undesired degradation of either or both may lead to nonspecific cytotoxicity. The IKZF proteins are zinc finger transcription factors that are involved with cell fate during hematopoiesis, and degradation of these proteins has been associated with hematotoxicity. See, e.g., Moreau, Kevin, et al. British Journal of Pharmacology 177.8 (2020): 1709-1718.

In some embodiments, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, can exhibit potent and selective induction of degradation of a BCL6 protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutically acceptable salt thereof, can selectively target a BCL6 protein for degradation over a second protein (e.g., GSPT1, IKZF1, IKZF2, IKZF3, CK1α, C6orf132, CAMP (cathelicidin antimicrobial peptide), CCNA2 (cyclin-A2), FSP1 (ferroptosis suppressor protein 1, also known as AIFM2), JCHAIN (immunoglobin J chain), NLRP7 (NACHT, LRR, PYD domains-containing protein 7), PTTG1 (securin), and/or TPX2 (targeting protein for Xklp2)). CAMP is an antimicrobial protein that is an integral part of the innate immune system, and it binds to bacterial lipopolysaccharides. CCNA2 controls both the G1/S and the G2/M transition phases of the cell cycle. FSP1 is an oxidoreductase that is an inhibitor of ferroptosis. JCHAIN links two monomer units of either IgM or IgA; the J chain-joined dimer is a nucleating unit of the IgM pentamer, and the J chain-joined dimer of IgA induces dimers or larger polymers. NLRP7 Inhibits CASP1/caspase-1-dependent IL1B secretion. PTTG1 is key for chromosomal stability and negatively regulates TP53. TPX2 is required for the normal assembly of mitotic spindles.

As used herein, “selective” or “selectively”, when referring to a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay.

In some embodiments, the compounds provided herein can exhibit potency (e.g., nanomolar potency) against a BCL6 protein with minimal activity (e.g., single digit micromolar potency, for example, potency greater than 1 μM (e.g., greater than 3 μM, 5 μM, 10 μM, 20 μM, or 30 μM)) against a second protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading (e.g., as measured by Yₘᵢₙ, DC₅₀, and/or Dₘₐₓ values) a second protein (e.g., GSPT1, IKZF1, IKZF2, IKZF3, CK1α, C6orf132, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit greater induction of degradation of a BCL6 protein relative to induction of degradation (e.g., as measured by Yₘᵢₙ, DC₅₀, and/or Dₘₐₓ values) of a second protein (e.g., GSPT1, IKZF1, IKZF2, IKZF3, CK1α, C6orf132, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit up to 1000-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 2-fold to about 10-fold greater induction of degradation of a BCL6 protein relative to induction of degradation of a second protein (e.g., GSPT1, IKZF1, IKZF2, IKZF3, CK1α, C6orf132, CAMP, CCNA2, FSP1, JCHAIN, NLRP7, PTTG1, and/or TPX2) (e.g., as measured by Y min, DC₅₀, and/or Dₘₐₓ values). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 10-fold to about 100-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 100-fold to about 1000-fold greater induction of degradation of a BCL6 protein relative to induction of degradation of a second protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 1000-fold to about 10000-fold greater induction of degradation of a BCL6 protein relative to induction of degradation of a second protein. In some embodiments, the second protein is selected from the group consisting of GSPT1, IKZF1, IKZF2, IKZF3, CK1α, C6orf132, CAMP, CCNA2, FSP1, JCHAIN, NLRP7, PTTG1, and TPX2. In some embodiments, the second protein is selected from the group consisting of GSPT1, IKZF1, IKZF2, IKZF3, and CKla. In some embodiments, the second protein is selected from the group consisting of C6orf132, CAMP, CCNA2, FSP1, JCHAIN, NLRP7, PTTG1, and TPX2. In some embodiments, the second protein is C6orf132. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with similar activity against a second protein (i.e., less than 2-fold greater activity against a BCL6 protein than against a second protein and no more than 2-fold greater activity against the second protein than against the BCL6 protein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit similar induction of degradation of a BCL6 protein relative to induction of degradation (e.g., as measured by Yₘᵢₙ, DC₅₀, and/or Dₘₐₓ values) of a second protein (i.e., less than 2-fold difference greater induction of degradation of a BCL6 protein than induction of degradation of a second protein and no more than 2-fold greater activity against the second protein than against a BCL6 protein) (e.g., GSPT1, IKZF1, IKZF2, IKZF3, CK1α, C6orf132, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit less than 2-fold greater induction of degradation of a BCL6 protein relative to induction of degradation of a second protein. In some embodiments, the second protein is selected from the group consisting of GSPT1, IKZF1, IKZF2, IKZF3, CK1α, C6orf132, CAMP, CCNA2, FSP1, JCHAIN, NLRP7, PTTG1, and TPX2. In some embodiments, the second protein is selected from the group consisting of GSPT1, IKZF1, IKZF2, IKZF3, and CK1α. In some embodiments, the second protein is selected from the group consisting of C6orf132, CAMP, CCNA2, FSP1, JCHAIN, NLRP7, PTTG1, and TPX2. In some embodiments, the second protein is C6orf132. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with minimal activity against a second protein (e.g., as measured by a proteomics assay, for example, a <20% reduction in protein abundance as measured in the proteomics assay described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading (e.g., as measured by abundance in a proteomic assay) a second protein (e.g., C6orf132, CAMP, CCNA2, FSP1, JCHAIN, NLRP7, PTTG, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit greater induction of degradation of a BCL6 protein relative to induction of degradation (e.g., as measured by abundance in a proteomic assay, for example, the proteomics assay as described herein) of a second protein (e.g., C6orf132, CAMP, CCNA2, FSP1, JCHAIN, NLRP7, PTTG1, and/or TPX2). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit up to 1000-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 2-fold to about 10-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 10-fold to about 100-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 100-fold to about 1000-fold greater induction of degradation of a BCL6 protein relative to induction of degradation of a second protein (e.g., C6orf132, CAMP, CCNA2, FSP1, JCHAIN, NLRP7, PTTG1, and/or TPX2) (e.g., as measured by abundance in a proteomic assay, for example, the proteomics assay as described herein). In some embodiments, the second protein is selected from the group consisting of GSPT1, IKZF1, IKZF2, IKZF3, CK1α, C6orf132, CAMP, CCNA2, FSP1, JCHAIN, NLRP7, PTTG1, and TPX2. In some embodiments, the second protein is selected from the group consisting of GSPT1, IKZF1, IKZF2, IKZF3, and CK1α. In some embodiments, the second protein is selected from the group consisting of C6orf132, CAMP, CCNA2, FSP1, JCHAIN, NLRP7, PTTG1, and TPX2. In some embodiments, the second protein is C6orf132. In some embodiments, the second protein is CAMP. In some embodiments, the second protein is CCNA2. In some embodiments, the second protein is FSP1. In some embodiments, the second protein is JCHAIN. In some embodiments, the second protein is NLRP7. In some embodiments, the second protein is PTTG1. In some embodiments, the second protein is TPX2.

In some embodiments, the compounds provided herein can exhibit potency against a BCL6 protein with minimal activity against any other detectable protein (e.g., as measured by a proteomics assay, for example, a <20% reduction in protein abundance as measured in the proteomics assay described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading (e.g., as measured by abundance in a proteomic assay, for example, the proteomics assay as described herein) any other detectable protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit greater induction of degradation of a BCL6 protein relative to induction of degradation (e.g., as measured by abundance in a proteomic assay, for example, the proteomics assay as described herein) of any other detectable protein. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit at least 2-fold, 3-fold, 5-fold, 10-fold, 25-fold, 50-fold, or 100-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit up to 1000-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 2-fold to about 10-fold greater induction of degradation of a BCL6 protein relative to induction of degradation of any other detectable protein (e.g., as measured by abundance in a proteomic assay, for example, the proteomics assay as described herein). In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit from about 10-fold to about 100-fold greater induction of degradation of a BCL6 protein relative to induction of degradation of any other detectable protein (e.g., as measured by abundance in a proteomic assay, for example, the proteomics assay as described herein).

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can exhibit potent degradation of a BCL6 protein and have minimal potency in degrading one or more additional proteins as measured by abundance in a proteomic assay. An exemplary proteomic experiment follows. OCI-Ly1 (DSMZ: ACC 722) cells are incubated with 100 nM of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or dimethyl sulfoxide (DMSO), for six hours. The cells are then washed two times with phosphate buffered saline and collected. Cells are lysed to extract total proteins, and total proteins are prepared for mass spectrometry analysis according to the protocol for the EASYPEP™ MS Sample Prep Kit (Fisher Scientific). In brief, proteins are reduced with dithiothreitol, alkylated with iodoacetamide, and digested with Trypsin and LysC enzyme. The resulting peptides are labeled with TMTPRO™ 18plex reagents (Fisher Scientific) according to the manufacturer protocol. Labeled peptides from each sample are mixed together in equal volumes, and the peptide mixture is separated by basic reverse-phase chromatography. A total of 85 fractions are combined into 18 pooled fractions. The pooled fractions are dried with a centrivap and resuspended in 5% acetonitrile, 0.1% formic acid for mass spectrometry analysis. Peptide abundance is quantified by tandem mass spectrometry using a Vanquish Neo chromatography system (Fisher Scientific) and Orbitrap FUSION™ LUMOS™ mass spectrometer (Fisher Scientific). Briefly, two micrograms of total peptides are loaded on a two-centimeter C₈ trap column followed by a 50-centimeter C₁₈ column. Data-dependent acquisition is performed to obtain peptide sequence and abundance information. Peptide and protein abundances are determined using the PROTEOME DISCOVERER™ software and the Homo sapiens proteome database (TaxID 9606), and the results are filtered to FDR<0.01. Significance thresholds are set to p-value <0.001 and abundance fold-change <50%.

Provided herein is a method of treating a cancer in a subject in need thereof, the method comprising administering to the subject a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof as a monotherapy. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for the treatment of cancer, for example, any of the cancers provided herein.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a medicament for the treatment of cancer, for example, any of the cancers provided herein.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in the manufacture of a medicament for the treatment of cancer, for example, any of the cancers provided herein.

Provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament. Also provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament for the treatment of cancer, for example, any of the cancers provided herein.

Provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use in treating a cancer, for example, any of the cancers provided herein.

As used herein, treatment of a cancer can include treatment of a primary tumor (i.e., non-metastatic cancer) (e.g., as first, second, third, or later line of therapy, including, but not limited to, the relapsed/refractory setting), treatment of a metastatic (or secondary) tumor, neoadjuvant therapy (e.g., before treatment with an additional therapy or therapeutic agent, such as surgery, radiation, chemotherapy, or a line of therapy), adjuvant therapy (e.g., following treatment with an additional therapy or therapeutic agent, such as surgery, radiation, chemotherapy, or a line of therapy), or maintenance therapy (e.g., treatment following response to an additional therapy or therapeutic agent, such as surgery, radiation, chemotherapy, or a line of therapy).

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a primary tumor. In some embodiments, the subject is treatment naïve with respect to the cancer. In some embodiments, the subject has received one or more lines of therapy for the cancer. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a metastatic tumor. In some embodiments, the subject is treatment naïve with respect to the metastatic tumor. In some embodiments, the subject has received one or more lines of therapy for the secondary tumor. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used as neoadjuvant therapy. In some embodiments, the neoadjuvant therapy precedes surgery (e.g., surgical resection, such as partial surgical resection or complete, total, or full surgical resection). In some embodiments, the neoadjuvant therapy precedes radiation therapy. In some embodiments, the neoadjuvant therapy precedes chemotherapy.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is an adjuvant therapy. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), or both. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer. In some embodiments, the adjuvant therapy follows surgery (e.g., surgical resection, such as partial surgical resection or complete, total, or full surgical resection). In some embodiments, the adjuvant therapy follows radiation therapy. In some embodiments, the adjuvant therapy follows chemotherapy.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is a maintenance therapy. In some embodiments, the patient has received chemotherapy, cell-based therapy (e.g., adoptive cell therapy (e.g., CAR T therapy, cytokine-induced killer cells (CIKs), natural killer cells (e.g., CAR-modified NK cells)) or antibody-armed cell therapy), a stem cell transplant, or a combination thereof. In some embodiments, the patient has received R-CHOP, G-CHOP, R-EPOCH, CVP, CVAD, R², R-CODOX-M, R-IVAC, DA-EPOCH-R, cell-based therapy, or two or more thereof. In some embodiments, the patient has received a rituximab-containing regimen. In some embodiments, the patient has received an obinutuzumab-containing regimen. In some embodiments, the patient has received a mosunetuzumab-containing regimen. In some embodiments, the patient has received an epcoritamab-containing regimen. In some embodiments, the patient has received a stem cell transplant. In some embodiments, the patient has received a cell-based therapy (e.g., CAR T therapy). In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received one or more lines of systemic therapy for the cancer. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient who has received two or more lines of systemic therapy for the cancer.

As used herein, “monotherapy”, when referring to a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, means that the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor, liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic lymphocytic leukemia (CLL), acute lymphocytic leukemia (ALL) (e.g., Philadelphia chromosome positive ALL, Philadelphia chromosome negative ALL), acute myeloid leukemia (AML) (e.g., acute promyelocytic leukemia (APL)), chronic myeloid leukemia (CML)), lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL))), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, mesothelioma, non-small cell lung cancer (NSCLC)), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma), thymus cancer (e.g., thymoma), or a combination thereof.

In some embodiments, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, pheochromocytoma, paraganglioma), thyroid cancer (e.g., papillary thyroid cancer)), eye cancer (e.g., uveal cancer (e.g., uveal melanoma)), gastrointestinal cancer (e.g., bile duct cancer (e.g., cholangiocarcinoma), colorectal cancer (e.g., colon adenocarcinoma, rectal adenocarcinoma, mucinous adenocarcinoma, mucinous carcinoma), esophageal cancer (e.g., esophageal adenocarcinoma), liver cancer (e.g., hepatocellular carcinoma), pancreatic cancer (e.g., pancreatic adenocarcinoma), or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), or testicular cancer (e.g., testicular germ cell tumors)), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), or uterine cancer (e.g., uterine carcinosarcoma, uterine endometrioid carcinoma, uterine serous carcinoma, uterine papillary serous carcinoma, uterine corpus endometrial carcinoma)), head and neck cancer (e.g., head and neck squamous cell carcinoma), hematological cancer (e.g., leukemia (e.g., chronic lymphocytic leukemia (CLL), acute lymphocytic leukemia (ALL) (e.g., B-cell lineage ALL (B-ALL), Philadelphia chromosome positive ALL (e.g., Philadelphia chromosome positive B-ALL), Philadelphia chromosome negative ALL (e.g., Philadelphia chromosome negative B-ALL)), acute myeloid leukemia (AML), chronic myeloid leukemia (CML)) or lymphoma (e.g., Hodgkin lymphoma (e.g., nodular lymphocyte predominant Hodgkin lymphoma (NLPHL)), non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL)))), respiratory cancer (e.g., lung cancer (e.g., lung squamous cell carcinoma, lung adenocarcinoma, mesothelioma, non-small cell lung cancer (NSCLC))), sarcoma (e.g., leiomyosarcoma, myxofibrosarcoma), skin cancer (e.g., melanoma), thymus cancer (e.g., thymoma), or a combination thereof.

In some embodiments, the cancer is a hematological cancer (e.g., a lymphoma (e.g., diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), peripheral T-cell lymphoma (PTCL) (e.g., PTCL with a T follicular helper phenotype (PTPCL-TFH), angioimmunoblastic T-cell lymphoma (AITL), or PTCL not otherwise specified (PTCL-NOS)), small lymphocytic lymphoma (SLL), Burkitt lymphoma (BL), mantle cell lymphoma (MCL)) or a leukemia (e.g., chronic lymphocytic leukemia (CLL), acute lymphocytic leukemia (ALL) (e.g., B-cell lineage ALL (B-ALL), Philadelphia chromosome positive ALL (e.g., Philadelphia chromosome positive B-ALL), Philadelphia chromosome negative ALL (e.g., Philadelphia chromosome negative B-ALL)), chronic myeloid leukemia (CML))), breast cancer, gastrointestinal cancer, brain cancer (e.g., glioblastoma) or lung cancer (e.g., NSCLC). In some embodiments, the cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML). In some embodiments, the cancer is hematological cancer is DLBCL, FL, MCL, BL, PTCL, or ALL (e.g., B-ALL). In some embodiments, the cancer is FL or DLBCL. In some embodiments, the cancer is DLBCL, FL, MCL, or ALL (e.g., B-ALL). See, e.g., Leeman-Neill and Bhagat, Expert Opinion on Therapeutic Targets 22.2 (2018): 143-152; Mlynarczyk and Melnick. Immunological Reviews 288.1 (2019): 214-239; Hurtz, Christian, et al., Journal of Experimental Medicine 208.11 (2011): 2163-2174; Deb, Dhruba, et al. Cancer Research 77.11 (2017): 3070-3081; Cardenas, Mariano G., et al., Clinical Cancer Research 23.4 (2017): 885-893; Walker, Sarah R., et al., Oncogene 34.9 (2015): 1073-1082; International Publication Nos. WO 2021/080950, WO 2021/077010, and WO 2022/221673.

In some embodiments, the cancer is non-Hodgkin lymphoma (e.g., Burkitt lymphoma (BL), diffuse large B-cell lymphoma (DLBCL), diffuse histiocytic lymphoma (DHL), follicular lymphoma (FL), intravascular large B-cell lymphoma (IVLBCL), mantle cell lymphoma (MCL), peripheral T-cell lymphoma (PTCL) (e.g., PTCL with a T follicular helper phenotype (PTPCL-TFH)), or small lymphocytic lymphoma (SLL))). In some embodiments, the non-Hodgkin lymphoma is B-cell non-Hodgkin lymphoma. In some embodiments, the non-Hodgkin lymphoma is CD20-positive. In some embodiments, the non-Hodgkin lymphoma is CD20-positive B-cell non-Hodgkin lymphoma. In some embodiments, the patient has not been previously treated for the non-Hodgkin lymphoma. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with R-CHOP (RITUXAN® (rituximab), cyclophosphamide, hydroxydaunorubicin, vincristine, and prednisone), or G-CHOP (GAZYVAR (obinutuzumab)), cyclophosphamide, hydroxydaunorubicin, vincristine, and prednisone). In some cases, the patient has previously been treated with etoposide and R-CHOP (called R-EPOCH). In some cases, the patient has been previously treated with R-CHOP combined with lenalidomide, venetoclax, ibrutinib, acalabrutinib, obinutuzumab, polatuzumab, pembrolizumab, durvalumab, or mosunetuzumab. In some embodiments, the patient has been previously treated with cyclophosphamide, vincristine, and prednisone (CVP), with or without rituximab or obinutuzumab. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the non-Hodgkin lymphoma is non-progressing (including stable disease) non-Hodgkin lymphoma. In some embodiments, the non-Hodgkin lymphoma is relapsed or refractory non-Hodgkin lymphoma. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, cancer is a non-Hodgkin lymphoma, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy.

In some embodiments, the cancer is DLBCL. In some embodiments, the DLBCL is characterized by a BCL2 translocation, a BCL6 translocation, a CD79B mutation (e.g., H225Y, A205D, Y196del, Y196F, Y196D, Y207X, Y196N, A205fs, Y196S, Y196H, A205fs, T206fs, H194_E197delinsQ, E197G, K219T, E192fs, or Y196C), an EZH2 mutation (e.g., a Y646F, Y646N, A682G, or A692V mutation), a MYC translocation, a MYD88 mutation (e.g., a L265P mutation), a NOTCH1 mutation (e.g., Q2394X, Q2501X, Q2459X, Y2490X, G2427fs, Q2444X, P2514fs, or P2517S), a NOTCH2 mutation (e.g., Q2285K, S2136fs, Q2361X, P2288fs, L2415fs, G2410fs, Q2409X, S2388X, 12304fs, Q2364X, Q2360fs, S2395X, E2261fs, M2267fs, Q2285fs, R2400X, P2303fs, Q2285fs, A2273fs, K2133fs, Q2389X, E2399X, E2290X, Q2325X, Y2340X, Y2392X, or E2411fs), a TP53 mutation (e.g., R181C, E336A, R248W, P98fs, P152L, R280I, S149fs, P151H, G245D, Y236D, S127F, A161T, D148fs, M246I, Y126C, H179R, A159P, C238G, L93fs, Y220C, R283fs, G244D, G245S, E171X, R209X, T155_R156dup, E271K, R306X, G105D, L93fs, G262V, W53X, G244V, H214Y, R282W, R337C, Q331fs, R273G, R273C, C176Y, S215R, R213Q, I195T, G245R, I232T, R175H, Y126D, R273H, R196X, Y205C, C141Y, C229X, Y126N, P278S, P151S, Y236H, R282G, Y103X, V216M, G244S, G266E, V173A, V173fs, I254S, T125M, R3A2X, P152fs, Y205D, V274L, L257P, C135Y, C176R, Y234N, R248Q, G244R, Y234H, R248G, M237I, R213X, E258D, V173M, L252_1254del, L2521, Y234C, or C176F), 17p deletion, 18q gain, or a combination thereof. In some embodiments, the DLBCL has a BCL6 rearrangement, a NOTCH2 mutation (e.g., Q2285K, S2136fs, Q2361X, P2288fs, L2415fs, G2410fs, Q2409X, S2388X, 12304fs, Q2364X, Q2360fs, S2395X, E2261fs, M2267fs, Q2285fs, R2400X, P2303fs, Q2285fs, A2273fs, K2133fs, Q2389X, E2399X, E2290X, Q2325X, Y2340X, Y2392X, E2411fs), or a combination thereof. In some embodiments, the DLBCL is DLBCL having a germinal center B cell (GCB) cell of origin. In some embodiments, the DLBCL is a BN2-type DLBCL (e.g., having a BCL6 rearrangement and/or a NOTCH2 mutation). In some embodiments, the DLBCL is an EZB-type DLBCL (e.g., having an EZH2 mutation and/or a BCL2 translocation). In some embodiments, the DLBCL is a C1 genetic cluster DLBCL (e.g., having a BCL6 rearrangement and/or a NOTCH2 mutation). See, e.g., Schmitz, Roland, et al. New England Journal of Medicine 378.15 (2018): 1396-1407; Chapuy, Bjoern, et al. Nature Medicine 24.5 (2018): 679-690 for additional description of these classifications.

In some embodiments, the cancer is a FL. In some embodiments, the FL has a BCL2 translocation (e.g., a t (14;18) translocation). In some embodiments, the FL has an EZH2 mutation (e.g., a Y646F, Y646N, A682G, or A692V mutation). See, e.g., Kridel, Robert, Laurie H. Sehn, and Randy D. Gascoyne. The Journal of Clinical Investigation 122.10 (2012): 3424-3431. In some embodiments, the cancer is a FL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy.

In some embodiments, the cancer is a B-ALL. In some embodiments, the B-ALL has an MLL rearrangement (e.g., an MLL-Af4 fusion, an MLL-Af6 fusion, an MLL-Af9 fusion, an MLL-ENL fusion, or an MLL-PTD fusion), is pre-B cell receptor positive (Pre-BCR+), has the Philadelphia chromosome, is Philadelphia chromosome-like, is dependent on Ras signaling, has a BCL2 amplification, has a JAK2 mutation (with or without high cytokine receptor-like factor 2(CRLF2) expression), or a combination thereof. See, e.g., Knight, Thomas, and Julie Anne Elizabeth Irving. Frontiers in Oncology 4 (2014): 160; Geng, Huimin, et al. Cancer Cell 27.3 (2015): 409-425; Jain, Nitin, et al. Blood, 129.5 (2017): 572-581; and Hurtz, Christian, et al. Genes & Development 33.17-18 (2019): 1265-1279. In some embodiments, the cancer is a B-ALL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used in the treatment of patient having a B-ALL. In some embodiments, the B-ALL is a relapsed or refractory B-ALL after two or more lines of systemic therapy.

In some embodiments, the patient has previously been treated with another anticancer agent, a chemotherapeutic agent, surgery, radiation, a multi-kinase inhibitor, or a combination thereof.

In some embodiments, the cancer is a DLBCL. In some embodiments, the patient has not been previously treated for the DLBCL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with lenalidomide in combination with rituximab or obinutuzumab. In some embodiments, the patient has previously been treated with cyclophosphamide, vincristine and prednisone (CVP), optionally in combination with rituximab or obinutuzumab. In some embodiments, the patient has previously been treated with R-CHOP (RITUXAN® (rituximab), cyclophosphamide, hydroxydaunorubicin, vincristine, and prednisone), or G-CHOP (GAZYVAR (obinutuzumab)), cyclophosphamide, hydroxydaunorubicin, vincristine, and prednisone). In some cases, the patient has previously been treated with etoposide and R-CHOP (called R-EPOCH). In some cases, the patient has been previously treated with R-CHOP combined with lenalidomide, venetoclax, ibrutinib, acalabrutinib, obinutuzumab, polatuzumab, pembrolizumab, durvalumab, or mosunetuzumab. In some embodiments, the patient has been previously treated with a rituximab-containing regimen. In some embodiments, the patient has been previously treated with an obinutuzumab-containing regimen. In some embodiments, the patient has been previously treated with a mosunetuzumab-containing regimen. In some embodiments, the patient has been previously treated with an epcoritamab-containing regimen. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the DLBCL is non-progressing (including stable disease) DLBCL. In some embodiments, the DLBCL is relapsed or refractory DLBCL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a DLBCL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a DLBCL. In some embodiments, the DLBCL is a relapsed or refractory DLBCL after two or more lines of systemic therapy.

In some embodiments, the cancer is a FL. In some embodiments, the patient has not been previously treated for the FL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with lenalidomide in combination with rituximab or obinutuzumab (the combination with rituximab is sometimes called “R²”). In some embodiments, the patient has previously been treated with cyclophosphamide, vincristine and prednisone (CVP), optionally in combination with rituximab or obinutuzumab. In some embodiments, the patient has previously been treated with R-CHOP or G-CHOP. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the non-Hodgkin lymphoma is non-progressing (including stable disease) FL. In some embodiments, the FL is relapsed or refractory FL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a FL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a FL. In some embodiments, the FL is a relapsed or refractory FL after two or more lines of systemic therapy.

In some embodiments, the cancer is a BL. In some embodiments, the patient has not been previously treated for the BL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with R-CHOP or G-CHOP. In some embodiments, the patient has previously been treated with rituximab, cyclophosphamide, vincristine, doxorubicin, and methotrexate (R-CODOX-M). In some embodiments, the patient has previously been treated with rituximab, ifosfamide, etoposide, and cytarabine (R-IVAC). In some embodiments, the patient has previously been treated with rituximab with dose-adjusted etoposide, prednisolone, vincristine, cyclophosphamide, and doxorubicin (DA-EPOCH-R). In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the BL is non-progressing (including stable disease) BL. In some embodiments, the BL is relapsed or refractory BL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a BL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a BL. In some embodiments, the BL is a relapsed or refractory BL after two or more lines of systemic therapy.

In some embodiments, the cancer is a PTCL (e.g., PTCL with a T follicular helper phenotype (PTPCL-TFH), angioimmunoblastic T-cell lymphoma (AITL), or PTCL not otherwise specified (PTCL-NOS)). In some embodiments, the patient has not been previously treated for the PTCL. In some embodiments, the patient has previously received chemotherapy. In some embodiments, the patient has been previously treated with rituximab or obinutuzumab as a monotherapy or in combination with an additional therapy or therapeutic agent. In some embodiments, the patient has been previously treated with rituximab as a monotherapy or in combination with an additional therapy or therapeutic agent. In some embodiments, the patient has been previously treated with obinutuzumab as a monotherapy or in combination with an additional therapy or therapeutic agent. In some embodiments, the patient has previously been treated with rituximab or obinutuzumab monotherapy. In some embodiments, the patient has previously been treated with bendamustine in combination with rituximab or obinutuzumab. In some embodiments, the patient has previously been treated with lenalidomide in combination with rituximab or obinutuzumab (the combination with rituximab is sometimes called “R²”). In some embodiments, the patient has previously been treated with cyclophosphamide, vincristine and prednisone (CVP), optionally in combination with rituximab or obinutuzumab (R-CVP or G-CVP, respectively). In some embodiments, the patient has previously been treated with R-CHOP or G-CHOP. In some embodiments, the patient has received one or more lines of systemic therapy. In some embodiments, the patient has received two or more lines of systemic therapy. In some embodiments, the patient has previously been treated with a cell-based therapy (e.g., adoptive cell therapy (e.g., CAR T therapy, cytokine-induced killer cells (CIKs), natural killer cells (e.g., CAR-modified NK cells)) or antibody-armed cell therapy). In some embodiments, the PTCL is non-progressing (including stable disease) PTCL. In some embodiments, the PTCL is relapsed or refractory PTCL. In some embodiments, the patient is a patient who relapsed after, or is refractory to, a rituximab-containing regimen. In some embodiments, the patient is a patient who relapsed after, or is refractory to, an obinutuzumab-containing regimen. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a PTCL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of a patient having a PTCL. In some embodiments, the PTCL is a relapsed or refractory PTCL after two or more lines of systemic therapy.

In some embodiments, the cancer is B-ALL. In some embodiments, the B-ALL is Philadelphia chromosome positive B-ALL. In some embodiments, the B-ALL is Philadelphia chromosome negative B-ALL. In some embodiments, the patient has previously received chemotherapy. In some embodiments, the patient has previously been treated with at least one cycle of induction, consolidation, intensification, and optional maintenance. In some cases, induction therapy can include an anthracycline, vincristine, a corticosteroid, and cyclophosphamide. In some embodiments, the anthracycline is doxorubicin. In some embodiments, the corticosteroid is dexamethasone. In some cases, the combination of doxorubicin, vincristine, dexamethasone, and cyclophosphamide is known as CVAD. In some embodiments, induction therapy can further include a tyrosine kinase inhibitor (e.g., a BCR-ABL inhibitor for patients with this fusion). In some embodiments, induction therapy can further include asparaginase (e.g., for pediatric patients). In some cases, consolidation therapy can include methotrexate, cytarabine, vincristine, 6-mercaptopurine, 6-thioguanine, cyclophosphamide, and etoposide. In some embodiments, consolidation therapy can further include a tyrosine kinase inhibitor (e.g., a BCR-ABL inhibitor for patients with this fusion). In some embodiments, consolidation therapy can further include asparaginase (e.g., for pediatric patients). In some cases, intensification therapy can include an anthracycline, vincristine, a corticosteroid, and cyclophosphamide. In some embodiments, intensification therapy can further include a tyrosine kinase inhibitor (e.g., a BCR-ABL inhibitor for patients with this fusion). In some embodiments, intensification therapy can further include asparaginase (e.g., for pediatric patients). Typically, pediatric and young adult regimens include higher cumulative doses of asparaginase and vincristine but may have lower cumulative doses of anthracycline and cyclophosphamide compared to adult regimens. In any of these cycle phases, an anti-CD20 immunotherapy (e.g., rituximab) can be added for patients expressing the CD20 protein on the cells. See, e.g., Muffly, Lori, and Emily Curran. Hematology 2014, the American Society of Hematology Education Program Book 2019.1 (2019): 17-23. In some embodiments, the patient has received one or more lines of systemic therapy. In some embodiments, the patient has received two or more lines of systemic therapy. In some embodiments, the B-ALL is relapsed or refractory B-ALL. In some such embodiments, treatment effect can be measured by progression-free survival (PFS), event-free survival (EFS), overall survival (OS), time to treatment failure, response rate (e.g., overall response rate, complete response, partial response, or a combination thereof), duration of response, or a combination thereof. In some embodiments, the cancer is a B-ALL, and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in the treatment of patient having a B-ALL. In some embodiments, the B-ALL is a relapsed or refractory B-ALL after two or more lines of systemic therapy.

Also provided herein is a method of treating a subject having a cancer, wherein the method comprises:
administering a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as a monotherapy or in conjunction with a second anticancer agent to the subject.

In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.d. (once daily) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.d. (twice daily) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.d. (three times daily) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.i.d. (four times daily) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.o.d. (every other day) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.week (once weekly) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.w. (twice weekly) to the subject. In some embodiments of any of the methods of treating cancers provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.w. (three times weekly) to the subject.

BCL6 activity has also been implicated in autoimmunity. See, for example, Li, Qing, et al. European Journal of Immunology 50.4 (2020): 525-536; Pearce, Andrew C., et al. Journal of Biological Chemistry 297.2 (2021); Venkatadri, Rajkumar, et al. European Journal of Immunology 52.5 (2022): 825-834; Patel, Preeyam S., et al. Science Advances 8.25 (2022): eabo1782; Ding, Shu, Yu Rao, and Qianjin Lu. Cellular & Molecular Immunology 19.7 (2022): 863-865. Accordingly, also provided herein is a method of treating an autoimmune condition in a subject in need thereof, the method comprising administering to the subject a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for the treatment of an autoimmune condition, for example, any of the autoimmune conditions provided herein.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a medicament for the treatment of an autoimmune condition, for example, any of the autoimmune conditions provided herein.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in the manufacture of a medicament for the treatment of an autoimmune condition, for example, any of the autoimmune conditions provided herein.

Provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament for the treatment of an autoimmune condition, for example, any of the autoimmune conditions provided herein.

Provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use in treating an autoimmune condition, for example, any of the autoimmune conditions provided herein.

In some embodiments, the autoimmune condition is acquired hemophilia, Addison's disease, ankylosing spondylitis, anti-neutrophil cytoplasmic antibody associated vasculitis (ANCA vasculitis), anti-synthetase syndrome, atherosclerosis, autoimmune hemolytic anemia, autoimmune hepatitis, autoimmune sclerosing cholangitis, autoimmune thyroiditis, autoimmune uveitis, Crohn's disease, dermatomyositis, diffuse scleroderma, Goodpasture's syndrome, graft-versus-host disease (GVHD) (e.g., chronic graft-versus-host disease (cGVHD)), Graves' disease, Guillain-Barre syndrome, Hashimoto's thyroiditis, Hughes' syndrome, IgG4-related disease, immune thrombocytopeniaurpura (ITP), inflammatory bowel disease, limited scleroderma, multiple sclerosis, myasthenia gravis (MG), neuromyelitis optica spectrum disorders (NMOSD) (e.g., neuromyelitis optica (NMO)), pemphigoid, pemphigus, pernicious anemia, polymyositis, primary biliary cirrhosis, psoriasis, psoriatic arthritis, rheumatoid arthritis, seronegative spondyloarthropathies, Sjogren's syndrome, systemic lupus erythematosus, thrombocytopenia purpura, Type 1 diabetes, ulcerative colitis, vitiligo, or a combination thereof. In some embodiments, the autoimmune condition is rheumatoid arthritis, systemic lupus erythematosus, or a combination thereof. In some embodiments, the autoimmune condition is ANCA vasculitis, GVHD (e.g., cGVHD), myasthenia gravis, NMO, or a combination thereof. In some embodiments, the autoimmune condition is ANCA vasculitis, anti-synthetase syndrome, arthritis (e.g., rheumatoid arthritis or inflammatory arthritis), GVHD (e.g., cGVHD), IgG4-RD, lupus (e.g., lupus erythematosus), ITP, MG (e.g., muscle-specific tyrosine kinase (MuSK) positive MG), MS, NMOSD (e.g., NMO), pemphigus (e.g., pemphigus vulgaris), Sjogren's syndrome, or a combination thereof. In some embodiments, the autoimmune condition is ANCA vasculitis, anti-synthetase syndrome, GVHD (e.g., cGVHD), TIP, MG (e.g., muscle-specific tyrosine kinase (MuSK) positive MG), NMOSD (e.g., NMO), pemphigus, or a combination thereof. In some embodiments, the autoimmune condition is arthritis (e.g., rheumatoid arthritis or inflammatory arthritis), GVHD (e.g., cGVHD), IgG4-RD, lupus (e.g., lupus erythematosus), MG (e.g., muscle-specific tyrosine kinase (MuSK) positive MG), MS, NMOSD (e.g., NMO), pemphigus (e.g., pemphigus vulgaris), Sjogren's syndrome, or a combination thereof. See, e.g., Pearce, Andrew C., et al. Journal of Biological Chemistry 297.2 (2021); Ding, Shu, Yu Rao, and Qianjin Lu, Cellular & Molecular Immunology (2022): 1-3; Lee, Dennis S W, Olga L. Rojas, and Jennifer L. Gommerman, Nature Reviews Drug Discovery 20.3 (2021): 179-199; and International Publication Nos. WO 2020/014599; WO 2021/074620.

In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.d. (once daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.d. (twice daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.d. (three times daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.i.d. (four times daily) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.o.d. (every other day) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed q.week (once weekly) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed b.i.w. (twice weekly) to the subject. In some embodiments of any of the methods of treating autoimmune conditions provided herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is dosed t.i.w. (three times weekly) to the subject.

Also provided herein is a method of treating a lymphoproliferative disorder in a subject in need thereof, the method comprising administering to the subject a therapeutically effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition comprising a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for the treatment of a lymphoproliferative disorder, for example, any of the lymphoproliferative disorders provided herein.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, as a medicament for the treatment of a lymphoproliferative disorder, for example, any of the lymphoproliferative disorders provided herein.

Provided herein is use of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in the manufacture of a medicament for the treatment of a lymphoproliferative disorder, for example, any of the lymphoproliferative disorders provided herein.

Provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use as a medicament for the treatment of a lymphoproliferative disorder, for example, any of the lymphoproliferative disorders provided herein.

Provided herein is a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof, for use in treating a lymphoproliferative disorder, for example, any of the lymphoproliferative disorders provided herein.

In some embodiments, the lymphoproliferative disorder is Epstein-Barr Virus-associated lymphoproliferative disorder.

Also provided is a method for modulating (e.g., decreasing) BCL6 protein activity in a cell, comprising contacting the cell with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein. As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL6 protein in a cell, comprising contacting the cell with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the level of BCL6 protein is decreased by at least 30% (e.g., at least 40%, at least 50%, at least 60%, at least 70%, at least 80%, at least 90%, at least 95%, at least 97%, or at least 99%) compared to a cell not contacted with the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL6 protein in a cell, comprising contacting the cell with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL6 protein, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and a CRBN protein or fragment thereof in a cell, comprising contacting the cell with a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, to a subject having a cell having a BCL6 protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method for inducing degradation of a BCL6 protein in a mammalian cell, the method comprising contacting the mammalian cell with an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof.

Also provided herein is a method of inhibiting cell proliferation, in vitro or in vivo, the method comprising contacting a cell with an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof as defined herein.

Further provided herein is a method of increasing cell death, in vitro or in vivo, the method comprising contacting a cell with an effective amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or a pharmaceutical composition thereof as defined herein. Also provided herein is a method of increasing tumor cell death in a subject. The method comprises administering to the subject a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, in an amount effective to increase tumor cell death.

When employed as pharmaceuticals, the compounds of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or pharmaceutically acceptable salts thereof, can be administered in the form of pharmaceutical compositions as described herein.

### Combinations

In any of the indications described herein, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be used as a monotherapy. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, can be used prior to administration of an additional therapeutic agent or additional therapy. For example, a subject in need thereof can be administered one or more doses of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time and then undergo at least partial resection of the tumor. In some embodiments, the treatment with one or more doses of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, for a period of time and undergo one or more rounds of radiation therapy. In some embodiments, the treatment with one or more doses of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, reduces the size of the tumor (e.g., the tumor burden) prior to the one or more rounds of radiation therapy.

In some embodiments of any the methods described herein, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered in combination with a therapeutically effective amount of at least one additional therapeutic (e.g., chemotherapeutic) agent.

Non-limiting examples of additional therapeutic agents include: RAS pathway targeted therapeutic agents (e.g., Ras/RAF/MEK/PI3K pathway inhibitors, (e.g., Ras inhibitors, KRas-targeted therapeutic agents, SOS1 inhibitors, SOS1/Ras protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, AKT inhibitors, BTK inhibitors, mTOR inhibitors, CDK4/5 inhibitors, CDK4/6 inhibitors, MET inhibitors, JAK inhibitors (e.g., JAK2 inhibitors), FAK inhibitors, ErbB family inhibitors (e.g., EGFR inhibitors, Her2 inhibitors), Src inhibitors), menin inhibitors, mTORC1 inhibitors, YAP inhibitors, proteasome inhibitors, farnesyl transferase inhibitors, HSP90 inhibitors, PTEN inhibitors, inhibitors of the polycomb repressive complex 2 (PRC2) (e.g., EZH1/2 or EZH2 inhibitors), signal transduction pathway inhibitors, checkpoint inhibitors, modulators of the apoptosis pathway (e.g., BCL-2 inhibitors, BCL-XL inhibitors), XPO1 inhibitors, steroids, chemotherapeutics, angiogenesis-targeted therapies, immune-targeted agents including immunomodulatory imide drugs (sometimes called “IMiDs” or “CELMoDs”), immunotherapy (e.g., anti-PD1, anti-PD-L1, anti-CD19, anti-CD20, anti-CD22, anti-CD3, anti-CD30, anti-CD79B, or anti-CD47 therapies, including antibodies (e.g., single-targeted antibodies targeting one or more of PD1, PD-L1, CD19, CD20, CD22, CD3, CD30, CD79B, or CD47, bispecific antibodies (including bispecific T cell engagers (BiTEs)) targeting one or more of PD1, PD-L1, CD19, CD20, CD22, CD3, CD30, CD79B, or CD47, and antibody-drug conjugates (ADCs) incorporating one or more of PD1, PD-L1, CD19, CD20, CD22, CD3, CD30, CD79B, or CD47 antibodies) or antigen-binding fragments thereof, a PD-1 inhibitor, or a PD-L1 inhibitor), cell-based therapeutics (e.g., adoptive cell therapy (e.g., CAR T therapy, cytokine-induced killer cells (CIKs), natural killer cells (e.g., CAR-modified NK cells)) or antibody-armed cell therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapeutic agent is a PI3K inhibitor, an Abl inhibitor (e.g., a BCR-Abl inhibitor), a BTK inhibitor, a JAK inhibitor (e.g., a JAK2 inhibitor), a BRaf inhibitor, a MEK inhibitor, a menin inhibitor, a BCL-2 inhibitor, a BCL-XL inhibitor, an MCL-1 inhibitor, an XPO1 inhibitor, an inhibitor of the polycomb repressive complex 2 (e.g., an EZH1/2 or EZH2 inhibitor), an immunomodulatory imide drug, a steroid, anti-CD19 therapy, anti-CD20 therapy, anti-CD3 therapy, chemotherapy, or a combination thereof.

Without being bound by any particular theory, it is believed that targeting BCL6 can result in the induction of genes that it typically represses, such as BCL2. In some embodiments, the additional therapeutic agent is a BCL-2 inhibitor (e.g., venetoclax, navitoclax, lisaftoclax, obatoclax, pelcitoclax, AZD-0466, BGB-11417, UBX-1325, UBX-1967, ZN-d5, oblimersen (e.g., oblimersen sodium), or beclanorsen).

In some embodiments, the PI3K inhibitor is alpelisib (BYL719), amdizalisib, apitolisib (GDC-0980), bimiralisib, buparlisib (BKM120), copanlisib (ALIQOPA™, BAY80-6946) (e.g., copanlisib dihydrochloride or a hydrate of copanlisib dihydrochloride), dactolisib (NVP-BEZ235, BEZ-235), dezapelisib, dordaviprone, duvelisib (e.g., a hydrate of duvelisib), eganelisib, fimepinostat, gedatolisib (PF-05212384, PKI-587), idelalisib, inavolisib, leniolisib (e.g., leniolisib phosphate), linperlisib, omipalisib (GSK2126458, GSK458), parsaclisib, pictilisib (GDC-0941), pilaralisib (XL147, SAR245408), paxalisib, rigosertib, risovalisib, seletalisib, serabelisib (TAK-117, MLN1117, INK 1117), sonolisib (PX-866), taselisib (GDC-0032, RG7604), umbralisib (e.g., umbralisib tosylate), voxtalisib (XL756, SAR245409), wortmannin, zandelisib, AMG 511, AMG319, ASN003, AZD8835, BGT-226 (NVP-BGT226), CH5132799, CUDC-907, GDC-0077, GDC-0084 (RG7666), GS-9820, GSK1059615, GSK2636771, KIN-193 (AZD-6428), LY2023414, LY294002, PF-04691502, PI-103, PKI-402, PQR309, SAR260301, SF1126, SHC-014748-M, TQ-B-3525, VS-5584 (SB2343), WX-037, XL-765, ZSTK474, or a combination thereof. In some embodiments, the PI3K inhibitor is alpelisib, amdizalisib, apitolisib, bimiralisib, buparlisib, copanlisib (e.g., copanlisib dihydrochloride or a hydrate of copanlisib dihydrochloride), dactolisib, dezapelisib, dordaviprone, duvelisib (e.g., a hydrate of duvelisib), eganelisib, fimepinostat, gedatolisib, idelalisib, inavolisib, leniolisib (e.g., leniolisib phosphate), linperlisib, parsaclisib, paxalisib, risovalisib, seletalisib, serabelisib, sonolisib, tenalisib, umbralisib (e.g., umbralisib tosylate), zandelisib, PF-04691502, SHC-014748-M, TQ-B-3525, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IKT-148009, NPB-001-056, or a combination thereof.

In some embodiments, the cancer is a B-ALL, and the additional therapy or therapeutic agent is an Abl inhibitor. In some embodiments, the cancer is a Philadelphia chromosome positive B-ALL and the additional therapy or therapeutic agent is an Abl inhibitor. In some embodiments, the cancer is a Philadelphia chromosome-like B-ALL, and the additional therapy or therapeutic agent is an Abl inhibitor. In some embodiments, the Abl inhibitor is selected from the group consisting of imatinib, dasatinib, ponatinib, or a combination thereof.

In some embodiments, the BTK inhibitor is abivertinib, acalabrutinib, atuzabrutinib, branebrutinib, dasatinib (e.g., dasatinib monohydrate), edralbrutinib (SHR-1459), elsubrutinib, evobrutinib, fenebrutinib, ibrutinib, luxeptinib, nemtabrutinib, olafertinib, nemtabrutinib, orelabrutinib, pirtobrutinib, remibrutinib, rilzabrutinib, spebrutinib, sunvozertinib, tirabrutinib (e.g., tirabrutinib hydrochloride), tolebrutinib, vecabrutinib, zanubrutinib, AC-0058 (AC-0058TA), BMS-986142, CT-1530, DTRMWXHS-12, LY-3337641 (HM-71224), M-7583, TAS-5315, or a combination thereof. In some embodiments, the BTK inhibitor is abivertinib, acalabrutinib, atuzabrutinib, branebrutinib, dasatinib (e.g., dasatinib monohydrate), edralbrutinib, elsubrutinib, evobrutinib, fenebrutinib, ibrutinib, nemtabrutinib, orelabrutinib, pirtobrutinib, remibrutinib, rilzabrutinib, sunvozertinib, tirabrutinib (e.g., tirabrutinib hydrochloride), tolebrutinib, zanubrutinib, AC-0058, BMS-986142, DTRMWXHS-12, LY-3337641, TAS-5315, or a combination thereof.

In some embodiments, the cancer is a B-ALL, and the additional therapy or therapeutic agent is a BTK inhibitor. In some embodiments, the cancer is a pre-BCR+B-ALL, and the additional therapy or therapeutic agent is a BTK inhibitor. In some embodiments, the cancer is a B-ALL dependent on Ras signaling, and the additional therapy or therapeutic agent is a BTK inhibitor. In some embodiments, the BTK inhibitor is ibrutinib or acalabrutinib.

In some embodiments, the JAK inhibitor is abrocitinib, baricitinib, brepocitinib, decernotinib, delgocitinib, deuruxolitinib, elsubrutinib, fedratinib (e.g., fedratinib dihydrochloride monohydrate), filgotinib (e.g., filgotinib maleate), gandotinib, gusacitinib, ilginatinib, itacitinib, ivarmacitinib, izencitinib, jaktinib, momelotinib, nezulcitinib, pacritinib (e.g., pacritinib citrate), peficitinib (e.g., peficitinib hydrobromide), povorcitinib (INCB-54707), ropsacitinib, ruxolitinib (e.g., ruxolitinib phosphate), solcitinib, tasocitinib (e.g., tofacitinib citrate), tinengotinib, upadacitinib (e.g., upadacitinib hydrate), zasocitinib, AGA-201, ATI-1777, ATI-501, ESK-001, GLPG-3667, INCB-52793, LNK-01001, LNK-01003, R-348, TD-8236, TLL-018, TQ-05105, VTX-958, or a combination thereof. In some embodiments, the JAK inhibitor is abrocitinib, baricitinib, brepocitinib, decernotinib, delgocitinib, deuruxolitinib, fedratinib (e.g., fedratinib dihydrochloride monohydrate), filgotinib (e.g., filgotinib maleate), gandotinib, gusacitinib, ilginatinib, itacitinib, ivarmacitinib, izencitinib, jaktinib, momelotinib, nezulcitinib, pacritinib (e.g., pacritinib citrate), peficitinib (e.g., peficitinib hydrobromide), povorcitinib (INCB-54707), ropsacitinib, ruxolitinib (e.g., ruxolitinib phosphate), solcitinib, tasocitinib (e.g., tofacitinib citrate), tinengotinib, upadacitinib (e.g., upadacitinib hydrate), zasocitinib, AGA-201, ATI-1777, ATI-501, ESK-001, GLPG-3667, LNK-01001, LNK-01003, R-348, TD-8236, TLL-018, TQ-05105, VTX-958, or a combination thereof.

In some embodiments, the cancer is a B-ALL, and the additional therapy or therapeutic agent is a JAK inhibitor. In some embodiments, the cancer is a JAK2 (e.g., JAK2R683G or JAK21682F) mutant B-ALL, and the additional therapy or therapeutic agent is a JAK inhibitor. In some embodiments, the cancer is a JAK2 (e.g., JAK2R683G or JAK21682F) mutant B-ALL with high CRLF2 expression, and the additional therapy or therapeutic agent is a JAK inhibitor and a BCL-2 inhibitor (e.g., venetoclax).

In some embodiments, the BRaf inhibitor is avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, PLX-4720, or a combination thereof.

In some embodiments, the MEK inhibitor is avutometinib (RO5126766), binimetinib (e.g., cobimetinib fumarate, COTELLIC®), (MEKTOVI®, MEK162), cobimetinib mirdametinib (PD0325901), pimasertib (MSC1936369B), refametinib, selumetinib (e.g., selumetinib sulfate, AZD6244), trametinib (e.g., trametinib dimethyl sulfoxide, GSK-1120212), zapnometinib, CI1040 (PD184352), CS3006, FCN-159, GSK-1120212, NFX-179, PD98059, SHR7390, TAK-733, WX-554, or a combination thereof. In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide, GSK-1120212), zapnometinib, FCN-159, NFX-179, TAK-733, or a combination thereof.

In some embodiments, the menin inhibitor is revumenib (e.g., revumenib fumarate), ziftomenib, BMF-219, DS-1594, JNJ-6617, or a combination thereof.

In some embodiments, the cancer is a B-ALL, and the additional therapy or therapeutic agent is a menin inhibitor. In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is a menin inhibitor.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325, UBX-1967, ZN-d5, or a combination thereof. In some embodiments, the BCL-2 inhibitor is lisaftoclax, navitoclax, obatoclax, venetoclax, oblimersen (e.g., oblimersen sodium), beclanorsen, or a combination thereof.

In some embodiments, the cancer is a B-ALL, and the additional therapy or therapeutic agent is a BCL-2 inhibitor. In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is a BCL-2 inhibitor. In some embodiments, the cancer is a BCL2 amplified B-ALL, and the additional therapy or therapeutic agent is a BCL-2 inhibitor. In some embodiments, the BCL-2 inhibitor is venetoclax.

In some embodiments, the cancer is a DLBCL, and the additional therapy or therapeutic agent is a BCL-2 inhibitor. In some embodiments, the BCL-2 inhibitor is venetoclax.

In some embodiments, the cancer is a FL, and the additional therapy or therapeutic agent is a BCL-2 inhibitor. In some embodiments, the BCL-2 inhibitor is venetoclax.

In some embodiments, the cancer is an MCL, and the additional therapy or therapeutic agent is a BCL-2 inhibitor. In some embodiments, the BCL-2 inhibitor is venetoclax.

In some embodiments, the BCL-XL inhibitor is lisaftoclax, navitoclax, obatoclax, pelcitoclax, mirzotamab clezutoclax, ABBV-155, APG-1252-12A, AZD-0466, DT-2216, PA-15227, UBX-1325, UBX-1967, XZ-739, 753-B, or a combination thereof. In some embodiments, the BCL-XL inhibitor is lisaftoclax, navitoclax, obatoclax, or a combination thereof.

In some embodiments, the MCL-1 inhibitor is omacetaxine (e.g., omacetaxine mepesuccinate).

In some embodiments, the XPO1 inhibitor is eltanexor, felezonexor, selinexor, verdinexor, BIIB-100, JS-110, or a combination thereof. In some embodiments, the XPO1 inhibitor is selinexor.

In some embodiments, the inhibitor of the PRC2 is lirametostat, tazemetostat (e.g., tazemetostat hydrobromide), tulmimetostat (CPI-0209), valemetostat (e.g., valemetostat tosylate), EBI-2511, HH-2853, HM-97662, PF-6821497, SHR-2554, XNW-5004, or a combination thereof. In some embodiments, the inhibitor of the PRC2 is an EZH1/2 inhibitor, an EZH2 inhibitor, or a combination thereof. Non-limiting examples of EZH2 and/or EZH1/2 inhibitors are described in International Publication Nos. WO 2011/140325, WO 2012/005805, WO 2012/050532, WO 2012/118812, WO 2012/142513, WO 2012/142504, WO 2013/049770, WO 2013/039988, WO 2013/067300, WO 2015/141616, WO 2017/084494, WO 2018/210296, WO 2018/210302, WO 2019/091450, WO 2019/204490, WO 2019/226491, WO 2020/063863, WO 2020/171606, WO 2020/228591, WO 2021/016414, WO 2021/063332, WO 2021/063340, WO 2021/180235, and WO 2022/035303.

In some embodiments, the cancer is a DLBCL, and the additional therapy or therapeutic agent is an inhibitor of the PRC2 (e.g., an EZH1/2 or EZH2 inhibitor). In some embodiments, the cancer is a B-ALL (e.g., Philadelphia chromosome positive B-ALL, Philadelphia chromosome negative B-ALL, or B-ALL with an MLL rearrangement (e.g., an MLL-Af4 fusion, an MLL-Af6 fusion, an MLL-Af9 fusion, an MLL-ENL fusion, or an MLL-PTD fusion)), and the additional therapy or therapeutic agent is an inhibitor of the PRC2. In some embodiments, the cancer is a FL, and the additional therapy or therapeutic agent is an inhibitor of the PRC2 (e.g., an EZH1/2 or EZH2 inhibitor). In some embodiments, the cancer is an MCL, and the additional therapy or therapeutic agent is an inhibitor of the PRC2 (e.g., an EZH1/2 or EZH2 inhibitor).

An exemplary wild-type human EZH2 sequence is shown below. This is one of several isoforms of EZH2, and it will be understood that residue numbering may change based on the reference isoform.

SEQ ID NO: 1 (UniParc ID UPI000006D77C):
MGQTGKKSEKGPVCWRKRVKSEYMRLRQLKRFRRADEVKSMFSSNRQKIL
ERTEILNQEWKQRRIQPVHILTSVSSLRGTRECSVTSDLDFPTQVIPLKT
LNAVASVPIMYSWSPLQQNFMVEDETVLHNIPYMGDEVLDQDGTFIEELI
KNYDGKVHGDRECGFINDEIFVELVNALGQYNDDDDDDDGDDPEEREEKQ
KDLEDHRDDKESRPPRKFPSDKIFEAISSMFPDKGTAEELKEKYKELTEQ
QLPGALPPECTPNIDGPNAKSVQREQSLHSFHTLFCRRCFKYDCFLHRKC
NYSFHATPNTYKRKNTETALDNKPCGPQCYQHLEGAKEFAAALTAERIKT
PPKRPGGRRRGRLPNNSSRPSTPTINVLESKDTDSDREAGTETGGENNDK
EEEEKKDETSSSSEANSRCQTPIKMKPNIEPPENVEWSGAEASMFRVLIG
TYYDNFCAIARLIGTKTCRQVYEFRVKESSIIAPAPAEDVDTPPRKKKRK
HRLWAAHCRKIQLKKDGSSNHVYNYQPCDHPRQPCDSSCPCVIAQNFCEK
FCQCSSECQNRFPGCRCKAQCNTKQCPCYLAVRECDPDLCLTCGAADHWD
SKNVSCKNCSIQRGSKKHLLLAPSDVAGWGIFIKDPVQKNEFISEYCGEI
ISQDEADRRGKVYDKYMCSFLFNLNNDFVVDATRKGNKIRFANHSVNPNC
YAKVMMVNGDHRIGIFAKRAIQTGEELFFDYRYSQADALKYVGIEREMEI
P

In some embodiments, the steroid is dexamethasone, prednisone, or a combination thereof.

In some embodiments, the immunomodulatory imide drug is avadomide, lenalidomide, iberdomide, pomalidomide, thalidomide, CC-99282, or a combination thereof.

In some embodiments, the additional therapy or therapeutic agent is lenalidomide and rituximab or obinutuzumab.

In some embodiments, the anti-CD19 therapy is blinatumomab (e.g., BLINCYTO® (blinatumomab) or a biosimilar thereof), coltuximab ravtansine, inebilizumab (e.g., inebilizumab-cdon, or a biosimilar thereof), loncastuximab tesirine (e.g., loncastuximab tesirine-lpyl, or a biosimilar thereof), obexelimab, tafasitamab (e.g., tafasitamab-cxix, or a biosimilar thereof), dDT-2219, biosimilars thereof, or a combination thereof. In some embodiments, the anti-CD19 therapy is a bispecific antibody or antigen-binding fragment thereof (e.g., BLINCYTO® (blinatumomab) or a biosimilar thereof). In some embodiments, the anti-CD19 therapy is an anti-CD19 and anti-CD3 bispecific antibody or antigen-binding fragment thereof (e.g., BLINCYTO® (blinatumomab) or a biosimilar thereof). In some embodiments, the anti-CD19 therapy is an antibody-drug conjugate (e.g., coltuximab ravtansine, loncastuximab tesirine (e.g., loncastuximab tesirine-lpyl, or a biosimilar thereof)).

In some embodiments, the anti-CD20 therapy is divozilimab, epcoritamab (e.g., epcoritamab-bysp, or a biosimilar thereof), glofitamab (e.g., COLUMVI® (glofitamab), or a biosimilar thereof), ibritumomab tiuxetan (e.g., ZEVALIN® (ibritumomab tiuxetan), or a biosimilar thereof), mosunetuzumab (e.g., mosunetuzumab-axgb, or a biosimilar thereof), obinutuzumab (e.g., GAZYVAR (obinutuzumab), or a biosimilar thereof), ocrelizumab (e.g., OCREVUS® (ocrelizumab), or a biosimilar thereof), odronextamab, ofatumumab (e.g., ARZERRAR (ofatumumab), or a biosimilar thereof), plamotamab, rituximab (e.g., RITUXAN® (rituximab), or a biosimilar thereof (e.g., rituximab-abbs, rituximab-arrx, rituximab-pvvr, ACELLBIAR (rituximab), HALPRYZA® (rituximab), HANLIKON® (rituximab), RIXATHON® (rituximab), REDITUX™ (rituximab), Retuxira (rituximab), BI-695500, GB-241, Mabion-CD20, RTXM-83, SAIT-101), ublituximab (e.g., ublituximab-xiiy, or a biosimilar thereof), veltuzumab, zuberitamab, MIL-62, SCT-400, TQB-2303, biosimilars thereof, or a combination thereof. In some embodiments, the anti-CD20 therapy is a bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof. In some embodiments, the anti-CD20 therapy is an anti-CD20 and anti-CD3 bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof. In some embodiments, the anti-CD20 therapy is an antibody-drug conjugate (e.g., ibritumomab tiuxetan (e.g., ZEVALIN® (ibritumomab tiuxetan), or a biosimilar thereof).

In some embodiments, the additional therapy or therapeutic agent is rituximab. In some such embodiments, the combination of rituximab and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy. In some such embodiments, the cancer is a FL.

In some embodiments, the additional therapy or therapeutic agent is obinutuzumab. In some such embodiments, the combination of obinutuzumab and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy. In some such embodiments, the cancer is a FL.

In some embodiments, the cancer is a DLBCL, and the additional therapy or therapeutic agent is anti-CD20 therapy. In some embodiments, the anti-CD20 therapy is rituximab, obinutuzumab, or a combination thereof.

In some embodiments, the cancer is a FL, and the additional therapy or therapeutic agent is anti-CD20 therapy. In some embodiments, the anti-CD20 therapy is rituximab, obinutuzumab, or a combination thereof.

In some embodiments, the anti-CD22 therapy is an antibody-drug conjugate (e.g., inotuzumab ozogamicin.

In some embodiments, the cancer is a B-ALL, and the additional therapy or therapeutic agent is anti-CD22 therapy. In some embodiments, the anti-CD22 therapy is an anti-CD22 antibody-drug conjugate (e.g., inotuzumab ozogamicin, or a biosimilar thereof).

In some embodiments, the anti-CD3 therapy is blinatumomab (e.g., BLINCYTO® (blinatumomab), or a biosimilar thereof), catumaxomab, elranatamab, epcoritamab (e.g., epcoritamab-bysp, or a biosimilar thereof), ertumaxomab, glofitamab (e.g., COLUMVI® (glofitamab), or a biosimilar thereof), linvoseltamab, mosunetuzumab (e.g., mosunetuzumab-axgb, or a biosimilar thereof), odronextamab, otelixizumab, plamotamab, talquetamab, tarlatamab, tebentafusp (e.g., tebentafusp-tebn, or a biosimilar thereof), teclistamab (e.g., teclistamab-cqyv, or a biosimilar thereof), teplizumab (e.g., teplizumab-mzwv, or a biosimilar thereof), visilizumab, biosimilars thereof, or a combination thereof. In some embodiments, the anti-CD3 therapy is bispecific antibody or antigen-binding fragment thereof (e.g., blinatumomab (e.g., BLINCYTO® (blinatumomab), or a biosimilar thereof), catumaxomab, elranatamab, epcoritamab (e.g., epcoritamab-bysp, or a biosimilar thereof), ertumaxomab, glofitamab (e.g., COLUMVI® (glofitamab), or a biosimilar thereof), linvoseltamab, mosunetuzumab (e.g., mosunetuzumab-axgb, or a biosimilar thereof), plamotamab, odronextamab, talquetamab, tarlatamab, tebentafusp (e.g., tebentafusp-tebn, or a biosimilar thereof), teclistamab (e.g., teclistamab-cqyv, or a biosimilar thereof), biosimilars thereof, or a combination thereof. In some embodiments, the anti-CD3 therapy is an anti-CD3 and anti-CD19 bispecific antibody or antigen-binding fragment thereof (e.g., BLINCYTO® (blinatumomab) or a biosimilar thereof). In some embodiments, the anti-CD3 therapy is an anti-CD20 and anti-CD3 bispecific antibody or antigen-binding fragment thereof (e.g., epcoritamab (e.g., epcoritamab-bysp, or a biosimilar thereof), glofitamab (e.g., COLUMVI® (glofitamab), or a biosimilar thereof), mosunetuzumab (e.g., mosunetuzumab-axgb, or a biosimilar thereof), plamotamab, odronextamab, biosimilars thereof, or a combination thereof.

In some embodiments, the anti-CD30 therapy is brentuximab, brentuximab vedotin, iratumumab, AFM-13, biosimilars thereof, or a combination thereof. In some embodiments, the anti-CD30 therapy is a bispecific antibody or antigen-binding fragment thereof (e.g., AFM-13). In some embodiments, the anti-CD30 therapy is an antibody-drug conjugate (e.g., brentuximab vedotin, or a biosimilar thereof).

In some embodiments, the anti-CD79B therapy is polatuzumab (e.g., polatuzumab vedotin (e.g., polatuzumab vedotin-piiq, or a biosimilar thereof)), MGD-010, RG-7986, biosimilars thereof, or a combination thereof. In some embodiments, the anti-CD79B therapy is polatuzumab (e.g., polatuzumab vedotin (e.g., polatuzumab vedotin-piiq, or a biosimilar thereof)), MGD-010, biosimilars thereof, or a combination thereof. In some embodiments, the anti-CD79B therapy is a bispecific antibody or antigen-binding fragment thereof (e.g., MGD-010). In some embodiments, the anti-CD79B therapy is an antibody-drug conjugate (e.g., polatuzumab vedotin (e.g., polatuzumab vedotin-piiq, or a biosimilar thereof)).

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, AZD7709, biosimilars thereof, or a combination thereof. In some embodiments, the anti-PD1 therapy is a bispecific antibody or antigen-binding fragment thereof (e.g., cadonilimab, ivonescimab, rilvegostomig, tebotelimab, volrustomig, vudalimab, AZD7709, HX-009, RG-6139, biosimilars thereof, or a combination thereof). In some embodiments, the anti-PD1 therapy is an anti-PD1 and anti-CD47 bispecific antibody or antigen-binding fragment thereof (e.g., HX-009, or a biosimilar thereof).

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., IMFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof. In some embodiments, the anti-PD-L1 therapy is a bispecific antibody or antigen-binding fragment thereof (e.g., erfonrilimab, BNT-311, biosimilars thereof, or a combination thereof).

In some embodiments, the PD-L1 inhibitor is INCB-086550.

In some embodiments, the anti-CD47 therapy is lemzoparlimab, letaplimab, magrolimab, 6MW-3211, AO-176, CPO-107, HX-009, TTI-621, TTI-622, biosimilars thereof, or a combination thereof. In some embodiments, the anti-CD47 therapy is lemzoparlimab, magrolimab, HX-009, TTI-621, TTI-622, biosimilars thereof, or a combination thereof. In some embodiments, the anti-anti-CD47 therapy is a bispecific antibody or antigen-binding fragment thereof (e.g., HX-009). In some embodiments, the anti-CD47 therapy is an anti-CD47 and anti-PD1 bispecific antibody or antigen-binding fragment thereof (e.g., HX-009, or a biosimilar thereof).

In some embodiments, the cell-based therapy is adoptive cell therapy (e.g., CAR T therapy, cytokine-induced killer cells (CIKs), natural killer cells (e.g., CAR-modified NK cells)) or antibody-armed cell therapy. In some embodiments, the cell-based therapy is CAR T therapy. In some embodiments, the cell-based therapy is axicabtagene ciloleucel (e.g., YESCARTA® (axicabtagene ciloleucel), or a biosimilar thereof), brexucabtagene autoleucel (e.g., TECARTUS® (brexucabtagene autoleucel), or a biosimilar thereof), inaticabtagene autoleucel, lisocabtagene maraleucel (e.g., BREYANZI® (lisocabtagene maraleucel), or a biosimilar thereof), rapcabtagene autoleucel, relmacabtagene autoleucel (e.g., CARTEYVAR (relmacabtagene autoleucel), or a biosimilar thereof), tisagenlecleucel (e.g., KYMRIAH® (tisagenlecleucel), or a biosimilar thereof), varnimcabtagene autoleucel (e.g., IMN-003A, or a biosimilar thereof), zamtocabtagene autoleucel, BZ-019, CD19CAR CTL, CTL-119, TAK-007, XLCART-001, biosimilars thereof, or a combination thereof.

In some embodiments, the additional therapy or therapeutic agent is chemotherapy. In some embodiments, the chemotherapy is CVAD, hyperCVAD (cyclophosphamide, vincristine, doxorubicin, and dexamethasone), CHOP, R-CHOP, G-CHOP, EPOCH, R-EPOCH, Pola-R-CHP (polatuzumab vedotin, rituximab, cyclophosphamide, doxorubicin, and prednisone), R-CODOX-M, R-IVAC, DA-EPOCH-R, CVP, R-CVP, G-CVP, CVD (cyclophosphamide, vincristine, dacarbazine, including mini-CVD), bendamustine with rituximab or obinutuzumab, methotrexate-cytarabine, vincristine (with or without steroids (e.g., dexamethasone)), nelarabine, a hypomethylating agent (e.g., azacitidine and/or decitabine), CALGB8811, or pediatric-inspired multi-agent chemotherapy (e.g., GRAALL-2003, COG AALL-0434, CCG-1961, CALGB 10403, or the DFCI regimen).

In some embodiments, the cancer is a B-ALL, and the additional therapy or therapeutic agent is chemotherapy (e.g., CVAD). In some embodiments, the cancer is an MLL-rearranged (e.g., an MLL-Af4 fusion, an MLL-Af6 fusion, an MLL-Af9 fusion, an MLL-ENL fusion, or an MLL-PTD fusion) B-ALL, and the additional therapy or therapeutic agent is chemotherapy (e.g., CVAD). In some embodiments, the cancer is a Philadelphia chromosome positive B-ALL, and the additional therapy or therapeutic agent is chemotherapy (e.g., CVAD). In some embodiments, the cancer is a Philadelphia chromosome-like B-ALL, and the additional therapy or therapeutic agent is chemotherapy (e.g., CVAD). In some embodiments, the cancer is a pre-BCR+B-ALL, and the additional therapy or therapeutic agent is chemotherapy (e.g., CVAD). In some embodiments, the cancer is a B-ALL dependent on Ras signaling, and the additional therapy or therapeutic agent is a chemotherapy (e.g., CVAD).

In some embodiments, the cancer is a FL, and the additional therapy or therapeutic agent is R-CHOP. In some such embodiments, the combination of R-CHOP and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of R-CHOP and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, the cancer is a DLBCL, and the additional therapy or therapeutic agent is R-CHOP. In some such embodiments, the combination of R-CHOP and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of R-CHOP and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, the cancer is a DLBCL, and the additional therapy or therapeutic agent is R-EPOCH. In some such embodiments, the combination of R-EPOCH and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of R-EPOCH and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, the cancer is a DLBCL, and the additional therapy or therapeutic agent is Pola-R-CHP. In some such embodiments, the combination of Pola-R-CHP and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as treatment for a primary tumor. In some such embodiments, the combination of Pola-R-CHP and a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is used as maintenance therapy.

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof is used in combination with one or more steps of induction, consolidation, intensification, or maintenance in a chemotherapeutic regimen.

Also provided herein is a method of treating cancer, comprising administering to a subject in need thereof (a) a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and (b) an additional therapeutic agent, for simultaneous, separate or sequential use for the treatment of cancer, wherein the amounts of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are together effective in treating the cancer. In some embodiments, the method comprises administering (c) at least one pharmaceutically acceptable carrier.

In some such embodiments, the additional therapeutic agent is an inhibitor of the PRC2 (e.g., an EZH1/2 inhibitor or an EZH2 inhibitor (e.g., any of the EZH1/2 inhibitors or EZH2 inhibitors described herein)), and the cancer is a cancer having an EZH2 alteration. In an aspect of this embodiment, the EZH2 alteration is a mutation is at residue 27, residue 34, residue 59, residue 141, residue 162, residue 172, residue 197, residue 238, residue 239, residue 246, residue 395, residue 401, residue 452, residue 510, residue 516, residue 556, residue 583, residue 618, residue 644, residue 646, residue 682, residue 690, residue 692, residue 716, residue 732, residue 744, residue 745, or a combination thereof, relative to SEQ ID NO. 1. In another aspect of this embodiment, the EZH2 alteration is a translocation. In another aspect of this embodiment, the EZH2 mutation is R²⁷*, R^{3A}*, E59*, Q141*, E162*, V172Cfs*11, E197Rfs*12, E238*, E239*, E246*, G395Efs*29, E401Kfs*22, E401*, Y452*, K510Yfs*3, X516_splice, S556*, R583*, X618_splice, S644*, Y646F, Y646N, Y646S, R690G, R690H, A692V, F716Lfs*24, X732_splice, I744Mfs*25, E745Afs*24, EZH2-AUTS2, EZH2-TMEM176B, GALNT11-EZH2, or a combination thereof. In another aspect of this embodiment, the EZH2 mutation is at residue 646, residue 682, or residue 692 relative to SEQ ID NO. 1. In another aspect of this embodiment, the EZH2 mutation is Y646F, Y646N, A682G, or A692V relative to SEQ ID NO: 1. In another aspect of this embodiment, the cancer is a lymphoma (e.g., FL or DLBCL) and the EZH2 mutation is Y646F, Y646N, A682G, or A692V relative to SEQ ID NO: 1.

These additional therapeutic agents may be administered with one or more doses of the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, or pharmaceutical composition comprising a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as part of the same or separate dosage forms, via the same or different routes of administration, and/or on the same or different administration schedules according to standard pharmaceutical practice known to one skilled in the art. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are administered simultaneously as separate dosages. In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are administered as separate dosages simultaneously, separately, or sequentially in any order, in jointly therapeutically effective amounts, e.g., in daily or intermittent dosages. In some embodiments, the compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, and the additional therapeutic agent are administered simultaneously as a combined dosage. When administered simultaneously, the two agents can be administered as a single dosage form (e.g., a fixed dosage form) or as separate dosages (e.g., non-fixed dosage forms).

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, and refer to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

### Pharmaceutical Compositions and Administration

### General

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (I) (e.g., Formula (I-aa) (e.g., Formula (I-aa-1), (I-aa-2), (I-aa-3), or (I-aa-4)), Formula (I-a) (e.g., Formula (I-a-1), (I-a-2), (I-a-3), or (I-a-4)), Formula (I-bb) (e.g., Formula (I-bb-1) or (I-bb-2)), or Formula (I-b) (e.g., Formula (I-b-1) or (I-b-2))) or Formula (II), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Also provided herein is a method of preparing a compound of Formula (I-aa) as depicted in Scheme 1,
the method comprising reacting a compound of Formula (SII) with a compound of Formula (SI), to provide a compound of Formula (I-aa):

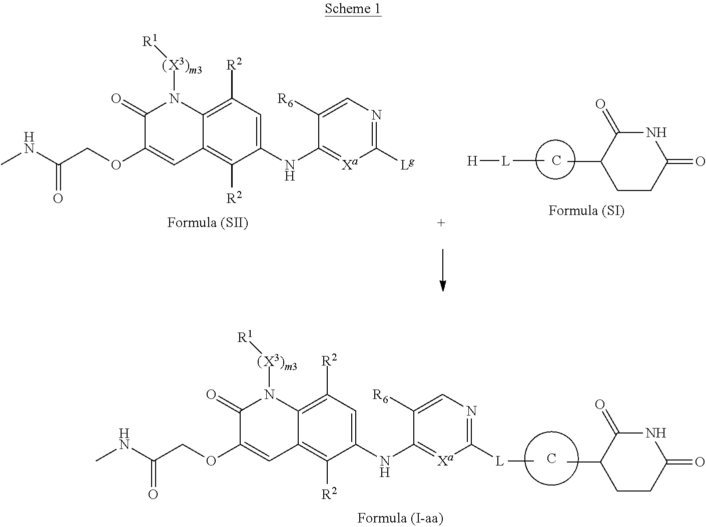

wherein:
L^{g} is halo;
X^{a} is N or CH;
R⁶ is —F or —Cl;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
m3 is 1, X³ is C₁₋₃ alkylene, and R¹ is H;
Ring C is selected from the group consisting of:

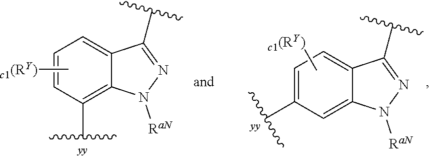

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:
-L^{A4}-L^{A1}-L^{A4}-_{bb};
-L^{A4}-O-L^{A4}-_{bb};
-L^{A4}-C(═O)-L^{A4}-_{bb}; and
-L^{A4}-L^{A1}-L^{A4}-C(═O)-_{bb},
wherein bb represents the point of attachment to Ring C;
L^{A1} is CH₂, CHMe, or CMe₂; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

In the methods of preparing compounds of Formula (I-aa) as depicted in Scheme 1, X³, m3, R¹, R², X^{a}, R⁶, L, and Ring C can be as defined herein for Formula (I-aa). In some embodiments of the methods, the compounds of Formula (I-aa) are compounds of Formula (I-aa-1). In some embodiments of the methods, the compounds of Formula (I-aa) are compounds of Formula (I-aa-2). In some embodiments of the methods, the compounds of Formula (I-aa) are compounds of Formula (I-aa-3). In some embodiments of the methods, the compounds of Formula (I-aa) are compounds of Formula (I-aa-4).

In some embodiments of the methods, the compounds of Formula (I-aa) are compounds of Formula (I-a). In some embodiments of the methods, the compounds of Formula (I-aa) are compounds of Formula (I-a-1). In some embodiments of the methods, the compounds of Formula (I-aa) are compounds of Formula (I-a-2). In some embodiments of the methods, the compounds of Formula (I-a) are compounds of Formula (I-a-3). In some embodiments of the methods, the compounds of Formula (I-aa) are compounds of Formula (I-a-4).

In some embodiments, the methods comprise reacting the compounds of Formula (SII) with the compounds of Formula (SI) under conditions suitable for S_{N}Ar reactions. In some embodiments, the methods comprise reacting the compounds of Formula (SII) with the compounds of Formula (SI) in the presence of a base (e.g., triethylamine or diisopropylethylamine) in an appropriate solvent. In some embodiments, the solvent is a polar aprotic solvent (e.g., DMSO or MeCN). In some embodiments, the methods comprise heating the compounds of Formula (SII) with the compounds of Formula (SI) at a temperature of at least 60° C. (e.g., heating the reaction at 60-135° C., 70-120° C., 70-110° C., or 80-110° C.).

Also provided herein are compounds of Formula (SI):

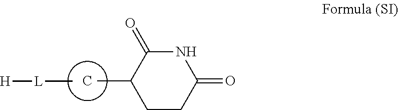

or salts thereof, wherein:
Ring C is selected from the group consisting of:

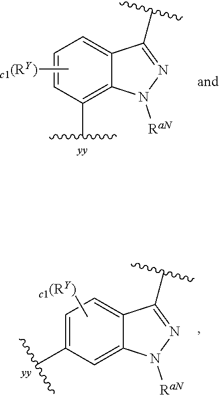

wherein: c1 is 0 or 1, R^{Y} is selected from the group consisting of halo (e.g., —F) and C₁₋₃ alkyl optionally substituted with 1-3 F, and R^{aN} is C₁₋₃ alkyl;
L is selected from the group consisting of:
-L^{A4}-L^{A1}-L^{A4}-_{bb};
-L^{A4}-O-L^{A4}-_{bb};
-L^{A4}-C(═O)-L^{A4}-_{bb}; and
-L^{A4}-L^{A1}-L^{A4}-C(═O)-_{bb},
wherein bb represents the point of attachment to Ring C;
L^{A1} is CH₂, CHMe, or CMe₂; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.

Also provided herein are compounds of Formula (SII):

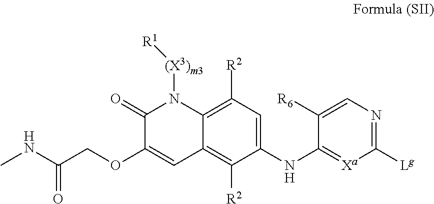

or salts thereof, wherein:
L^{g} is halo;
X^{a} is N or CH;
R⁶ is —F or —Cl;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
m3 is 1,
X³ is C₁₋₃ alkylene, and
R¹ is H.

### EXAMPLES

In some of the examples disclosed herein, the final product of a described chemical reaction sequence is structurally depicted with enhanced stereochemical notation(s) at one or more stereogenic center(s). Examples of such notations include or1, or2, and the like. In some such examples, in the chemical name of the same compound, each of such stereogenic center(s) is assigned a tentative configuration (e.g., (R)- or (S)-) based on the wedge/dash representation of the structural formula. However, the stereogenic center(s) should be understood to have configurations consistent with the enhanced stereochemical notation(s), as described herein.

Accordingly, unless otherwise specified, starting materials and intermediates leading to these compounds incorporate the enhanced stereochemical notations at the corresponding stereogenic centers, notwithstanding the tentative assignments provided in their chemical names.

For example, based on the or1 found on the stereogenic center, Compound 307b in Example 84 is a single stereoisomer selected from:
2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide; and
2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide.

The intermediate product provided in the first step of the same example incorporates the or1 notation. It is therefore a single stereoisomer selected from:
tert-butyl (R)-4-(1-(piperidin-4-yl)ethyl)piperazine-1-carboxylate; and
tert-butyl(S)-4-(1-(piperidin-4-yl)ethyl)piperazine-1-carboxylate.

For further clarification, a chemical name that takes into account the enhanced stereochemical notation(s) is provided for the final product having enhanced stereochemical notation(s) at one or more stereogenic centers. This chemical name is enclosed in brackets (i.e., “[ ]”). In these chemical names, the prefix “rel” means that the stereochemical configuration shown in a chemical name is relative. To illustrate, when a compound contains one stereogenic center, and its chemical name starts with the prefix “rel,” then this stereogenic center is resolved, but its absolute configuration is either (R)- or (S)-. As such, it should be labelled with an or1 enhanced stereochemical notation in its corresponding structure. For example, the chemical name that takes into account the enhanced stereochemical notation for Compound 307b is:
[rel-2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide], as described in Example 84.

### Example 1. Synthesis of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-methyl-2-oxo-1,2-dihydroquinolin-3-yl)oxy)-N-methylacetamide (Compound 101)

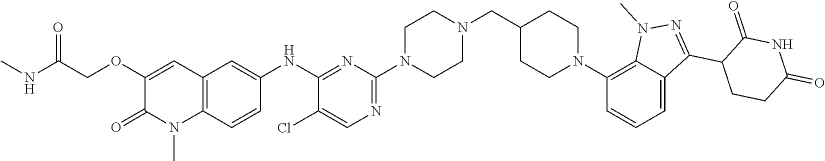 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)ccc2n(C)c1=O]

### Preparation of tert-butyl 4-((1-((benzyloxy)carbonyl)piperidin-4-yl)methyl)piperazine-1-carboxylate

To a stirred solution of tert-butyl piperazine-1-carboxylate (25.0 g, 0.13 mmol), benzyl4-formylpiperidine-1-carboxylate (39.60 g, 0.16 mmol), and acetic acid (7.80 g, 0.13 mmol) in DCM (500 mL) was added NaBH(OAc)₃ (42.5 g, 0.20 mmol) at 25° C., and the resulting mixture was stirred at 25° C. for 3 hours. The reaction mixture was quenched with water and extracted with DCM. The organic layer was separated, dried over anhydrous sodium sulfate, filtered and concentrated. Crude was purified by column chromatography (SiO₂, 100-200 mesh, 15% EtOAc in petroleum ether) to afford tert-butyl 4-((1-((benzyloxy)carbonyl)piperidin-4-yl)methyl)piperazine-1-carboxylate (25 g) as a pale-yellow solid.

LC-MS (ESI): m/z=418.12 [M+H]⁻.

### Preparation of tert-butyl 4-(piperidin-4-ylmethyl)piperazine-1-carboxylate

To a stirred solution of tert-butyl4-((1-((benzyloxy)carbonyl)piperidin-4-yl)methyl)piperazine-1-carboxylate (25 g, 0.06 mmol) in MeOH (500 mL) was added Pd/C (7.5 g), and the reaction mixture was stirred under hydrogen atmosphere. The resulting mixture was stirred at room temperature for 16 hours. The reaction mixture was diluted with 30% methanol in DCM, filtered through a pad of celite-bed, and the filtrate was collected and concentrated to afford crude tert-butyl 4-(piperidin-4-ylmethyl)piperazine-1-carboxylate. The crude was purified by trituration using hexane and diethyl ether to afford tert-butyl 4-(piperidin-4-ylmethyl)piperazine-1-carboxylate (7.0 g) as a yellow solid.

¹H NMR (400 MHZ, DMSO-d6): δ 3.29 (s, 4H), 3.11-3.08 (m, 2H), 2.66 (t, J=11.60 Hz, 2H), 2.27-2.25 (m, 4H), 2.11 (d, J=6.80 Hz, 2H), 1.74-1.71 (m, 3H), 1.39 (s, 9H), 1.17-1.14 (m, 2H).

### Preparation of 7-bromo-3-iodo-1-methyl-indazole

To a solution of 7-bromo-3-iodo-1H-indazole (20 g, 61.93 mmol, 1.0 eq.) in THF (50 mL) was added t-BuOK (13.90 g, 123.87 mmol, 2.0 eq.) at 0° C. for 1 hour. CH₃I (26.37 g, 185.80 mmol, 11.57 mL, 3.0 eq.) was added to the mixture at 0° C. The mixture was stirred at 20° C. for 2 hours under N₂ atmosphere. The reaction mixture was filtered and concentrated under reduced pressure to obtain a residue. The crude product was purified by reverse-phase HPLC (20-100% 50 min; 100% 20 minutes, 0.1% formic acid condition) to afford 7-bromo-3-iodo-1-methyl-indazole (15 g) as a yellow solid.

LC-MS (ESI): m/z=336.6 [M+H]⁺.

### Preparation of 7-bromo-3-(2,6-dibenzyloxy-3-pyridyl)-1-methyl-indazole

A mixture of 2,6-dibenzyloxy-3-(4,4,5,5-tetramethyl-1,3,2-dioxaborolan-2-yl)pyridine (17.34 g, 41.55 mmol, 1.0 eq.), 7-bromo-3-iodo-1-methyl-indazole (14 g, 41.55 mmol, 1.0 eq.), cyclopentyl(diphenyl)phosphane; dichloromethane; dichloropalladium; iron (6.79 g, 8.31 mmol, 0.2 eq.), and Cs₂CO₃ (40.61 g, 124.65 mmol, 3.0 eq.) in THF (200 mL) and H₂O (40 mL) was degassed and purged with N₂ 3 times. The mixture was stirred at 85° C. for 1 hour under N₂ atmosphere. The reaction mixture was concentrated under reduced pressure to remove solvent. The resulting residue was purified by column chromatography (SiO₂, petroleum ether/ethyl acetate=1/0 to 10/1) to afford 7-bromo-3-(2,6-dibenzyloxy-3-pyridyl)-1-methyl-indazole (15.1 g) as a white solid.

LC-MS (ESI): m/z=502.3 [M+H]⁺.

### Preparation of tert-butyl 4-((1-(3-(2,6-bis(benzyloxy)pyridin-3-yl)-1-methyl-1H-indazol-7-yl)piperidin-4-yl)methyl)piperazine-1-carboxylate

To a stirred solution of tert-butyl 4-(piperidin-4-ylmethyl)piperazine-1-carboxylate (0.34 g, 1.20 mmol) in 1,4-dioxane (10 mL) was added 7-bromo-3-(2,6-dibenzyloxy-3-pyridyl)-1-methyl-indazole (0.50 g, 1.00 mmol), and the mixture was purged with argon for 15 minutes; then cesium carbonate (0.98 g, 3.00 mmol) and Pd-PEPPSI-iHeptCl (0.05 g, 0.05 mmol) was added, and the reaction mixture heated to 120° C. for 3 hours. The reaction mixture quenched with water and extracted into ethyl acetate. The combined organic layers were washed with brine solution and dried over anhydrous Na₂SO₄, filtered, and concentrated under vacuum to get crude. The crude was purified by flash column chromatography (SiO₂, 100-200 mesh, 27% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl)piperazine-1-carboxylate (0.48 g) as a pale yellow solid.

LC-MS (ESI): m/z=703.5 [M+H]⁺.

### Preparation of 3-(2,6-bis(benzyloxy)pyridin-3-yl)-1-methyl-7-(4-(piperazin-1-ylmethyl)piperidin-1-yl)-1H-indazole hydrochloride

To a stirred solution of tert-butyl 4-((1-(3-(2,6-bis(benzyloxy)pyridin-3-yl)-1-methyl-1H-indazol-7-yl)piperidin-4-yl)methyl)piperazine-1-carboxylate (0.48 g, 0.68 mmol) in DCM (4.8 mL) was added 4 M HCl in dioxane (2.8 mL) at 0° C. and stirred at room temperature for 16 hours. The reaction mixture was concentrated under vacuum to get 3-(2,6-bis(benzyloxy)pyridin-3-yl)-1-methyl-7-(4-(piperazin-1-ylmethyl)piperidin-1-yl)-1H-indazole hydrochloride (0.45 g) as an off-white semi solid.

¹H NMR (400 MHZ, DMSO-d₆): δ 7.84 (d, J=8.00 Hz, 1H), 7.48 (d, J=1.60 Hz, 2H), 7.41-7.42 (m, 9H), 7.30-7.31 (m, 1H), 6.57-6.59 (m, 1H), 5.43 (d, J=5.60 Hz, 4H), 4.32 (s, 3H), 3.27-3.25 (m, 2H), 3.08 (s, 4H), 2.54-2.57 (m, 1H), 2.51-2.51 (m, 3H), 2.28-2.30 (m, 1H).

### Preparation of 3-(1-methyl-7-(4-(piperazin-1-ylmethyl)piperidin-1-yl)-1H-indazol-3-yl)piperidine-2,6-dione

To a stirred solution of 3-(2,6-bis(benzyloxy)pyridin-3-yl)-1-methyl-7-(4-(piperazin-1-ylmethyl)piperidin-1-yl)-1H-indazole hydrochloride (0.45 g, 0.75 mmol) in DMF (4.50 mL) was added 10% palladium on carbon (dry)(0.45 g), and the mixture was stirred under hydrogen purging with bladder pressure at room temperature for 16 hours. The reaction mixture was diluted with DCM and filtered through a pad of celite and washed with excess of 30% THF in DCM. The filtrate was collected and concentrated under vacuum to afford crude 3-(1-methyl-7-(4-(piperazin-1-ylmethyl)piperidin-1-yl)-1H-indazol-3-yl)piperidine-2,6-dione (0.45 g, crude) as a pale yellow solid. The crude product was triturated with methanol and diethyl ether to afford 3-(1-methyl-7-(4-(piperazin-1-ylmethyl)piperidin-1-yl)-1H-indazol-3-yl)piperidine-2,6-dione (0.45 g) as a pale yellow solid.

LC-MS (ESI): m/z=425 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-methyl-2-oxo-1,2-dihydroquinolin-3-yl)oxy)-N-methylacetamide

To a stirred suspension of 2-((6-((2,5-dichloropyrimidin-4-yl)amino)-1-methyl-2-oxo-1,2-dihydroquinolin-3-yl)oxy)-N-methylacetamide (0.14 g, 0.46 mmol)(see, e.g., International Publication No. WO 2018/108704) and N,N-diisopropylethylamine (0.8 mL, 4.64 mmol) in N,N-dimethylformamide (5 mL), 3-(1-methyl-7-(4-(piperazin-1-ylmethyl)piperidin-1-yl)-1H-indazol-3-yl)piperidine-2,6-dione (0.25 g, 0.58 mmol) was added at room temperature. The reaction mixture was heated at 100° C. for 5 hours. The reaction mixture was added to ice-cold water and a solid was precipitated out. The solid was filtered and dried under vacuum to obtain crude product. The crude product was purified by preparative HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-methyl-2-oxo-1,2-dihydroquinolin-3-yl)oxy)-N-methylacetamide as an off-white solid (0.029 g).

LC-MS (ESI): m/z=794.53 [M+H]⁺.

¹H NMR (400 MHZ, DMSO-d₆): δ 10.94 (s, 1H), 8.87 (s, 1H), 8.06 (s, 1H), 7.97-7.93 (m, 2H), 7.74 (dd, J=2.40, 9.00 Hz, 1H), 7.48 (d, J=9.20 Hz, 1H), 7.37-7.35 (m, 1H), 7.13 (s, 1H), 7.01-7.00 (m, 2H), 4.59 (s, 2H), 4.34-4.31 (m, 1H), 4.24 (s, 3H), 3.67-3.64 (m, 7H), 3.16 (br s, 2H), 2.67-2.60 (m, 7H), 2.41 (br s, 4H), 2.32-2.15 (m, 4H), 1.90-1.87 (m, 2H), 1.75 (br s, 1H), 1.39-1.35 (m, 2H).

### Example 2. Synthesis of 2-((6-((5-chloro-2-(4-((1-(3-(2,6-dioxopiperidin-3-yl)-1-methyl-1H-indazol-6-yl)piperidin-4-yl)methyl)piperazin-1-yl)pyrimidin-4-yl)amino)-1-methyl-2-oxo-1,2-dihydroquinolin-3-yl)oxy)-N-methylacetamide (Compound 102)

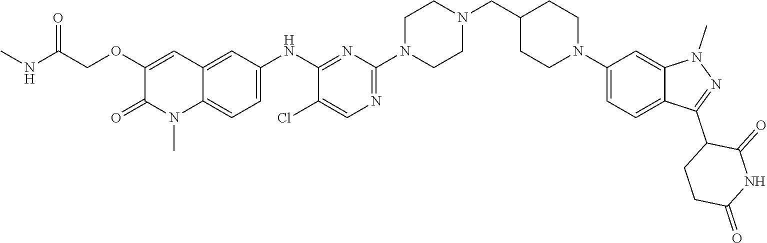 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)ccc2n(C)c1=O]

### Preparation of 6-bromo-3-iodo-1-methyl-indazole

To a solution of 6-bromo-3-iodo-1H-indazole (30 g, 92.90 mmol, 1 eq.) in DMF (200 mL) was added NaH (7.43 g, 185.80 mmol, 60% purity, 2 eq.) at 0° C. The mixture was stirred for 1 hour. After stirring, Mel (26.37 g, 185.80 mmol, 11.57 mL, 2 eq.) was added into the solution. The mixture was stirred at 20° C. for 1 hour. After completion, the solution was cooled to 0° C., and saturated NH₄Cl solution (300 mL) was added dropwise. The solution was extracted with ethyl acetate (500 mL×3). The combined organic layers were washed with brine (500 mL), dried over anhydrous sodium sulfate, filtered, and concentrated under reduced pressure to give a residue. The residue was purified by reverse-phase HPLC (0.1% formic acid condition) to afford 6-bromo-3-iodo-1-methyl-indazole (15.3 g) as a brown solid.

LC-MS (ESI): m/z=336.8 [M+H]⁺.

### Preparation of 6-bromo-3-(2,6-dibenzyloxy-3-pyridyl)-1-methyl-indazole

A mixture of 6-bromo-3-iodo-1-methyl-indazole (14.8 g, 43.92 mmol, 1 eq.), 2,6-dibenzyloxy-3-(4,4,5,5-tetramethyl-1,3,2-dioxaborolan-2-yl)pyridine (18.33 g, 43.92 mmol, 1 eq.), Pd(dppf)Cl₂ (3.21 g, 4.39 mmol, 0.1 eq.), Cs₂CO₃ (42.93 g, 131.77 mmol, 3 eq.), and H₂O (10 mL) in THF (100 mL) was degassed and purged with N₂ 3 times. The mixture was stirred at 60° C. for 16 hours under N₂ atmosphere. After completion, the reaction mixture was poured into water (200 mL) and extracted with ethyl acetate (150 mL×3). The combined organic layers were washed with brine (200 mL), dried over anhydrous sodium sulfate, filtered, and concentrated under reduced pressure to give a residue. The residue was purified by column chromatography (SiO₂, petroleum ether/ethyl acetate=100/1 to 20/1) to afford 6-bromo-3-(2,6-dibenzyloxy-3-pyridyl)-1-methyl-indazole (14 g) as a yellow solid.

¹H NMR (400 MHZ, DMSO-d₆) ¿=7.94 (d, J=1.2 Hz, 1H), 7.91 (d, J=8.0 Hz, 1H), 7.61 (d, J=8.4 Hz, 1H), 7.51-7.44 (m, 2H), 7.42-7.23 (m, 8H), 7.10 (dd, J=1.6, 8.8 Hz, 1H), 6.59 (d, J=8.0 Hz, 1H), 5.44 (d, J=8.4 Hz, 4H), 4.04 (s, 3H).

### Preparation of tert-butyl 4-((1-(3-(2,6-bis(benzyloxy)pyridin-3-yl)-1-methyl-1H-indazol-6-yl)piperidin-4-yl)methyl)piperazine-1-carboxylate

To a stirred suspension of 6-bromo-3-(2,6-dibenzyloxy-3-pyridyl)-1-methyl-indazole (1.0 g, 2.0 mmol) in 1,4-dioxane (15 mL) and DMF (15 mL) was added tert-butyl 4-(piperidin-4-ylmethyl)piperazine-1-carboxylate (0.68 g, 2.4 mmol) and cesium carbonate (1.95 g, 6.0 mmol). The reaction was purged with argon for 15 minutes; then RuPhos (0.18 g, 0.4 mmol) and RuPhos-Pd-G₃ (0.08 g, 0.1 mmol) was added, and the reaction was heated to 100° C. for 3 hours. The reaction mixture was diluted with water and extracted with ethyl acetate. The combined organic layers were washed with brine solution, dried over anhydrous Na₂SO₄, filtered, and concentrated to afford crude product. The crude was purified by flash column chromatography (Amine silica, 35-60 mesh, 50% EtOAc in petroleum ether) to afford tert-butyl 4-((1-(3-(2,6-bis(benzyloxy)pyridin-3-yl)-1-methyl-1H-indazol-6-yl)piperidin-4-yl)methyl)piperazine-1-carboxylate (0.7 g) as a brown semi-solid.

LC-MS (ESI): m/z=703.72 [M+H]⁺.

### Preparation of tert-butyl 4-((1-(3-(2,6-dioxopiperidin-3-yl)-1-methyl-1H-indazol-6-yl)piperidin-4-yl)methyl)piperazine-1-carboxylate

To a stirred solution of tert-butyl4-((1-(3-(2,6-bis(benzyloxy)pyridin-3-yl)-1-methyl-1H-indazol-6-yl)piperidin-4-yl)methyl)piperazine-1-carboxylate (0.70 g, 0.99 mmol) in mixture of isopropyl alcohol (10 mL), ethyl acetate (10 mL), and DMF (7 mL) was added 10% Pd/C (0.69 g, 6.50 mmol). The reaction was put under hydrogen atmosphere (balloon) for 12 hours at room temperature. The reaction mixture was filtered through a pad of celite using 50% THF-DCM. The filtrate was concentrated under reduced pressure to afford tert-butyl 4-((1-(3-(2,6-dioxopiperidin-3-yl)-1-methyl-1H-indazol-6-yl)piperidin-4-yl)methyl)piperazine-1-carboxylate (0.40 g) as a brown solid.

LC-MS (ESI): m/z=525.55 [M+H]⁺.

### Preparation of 3-(1-methyl-6-(4-(piperazin-1-ylmethyl)piperidin-1-yl)-1H-indazol-3-yl)piperidine-2,6-dione (HCl salt

To a stirred solution of tert-butyl 4-((1-(3-(2,6-dioxopiperidin-3-yl)-1-methyl-1H-indazol-6-yl)piperidin-4-yl)methyl)piperazine-1-carboxylate (0.40 g, 0.76 mmol) in DCM (4 mL) was added 4 M HCl in dioxane (2 mL). The reaction was stirred at room temperature for 3 hours. The reaction mixture was concentrated under reduced pressure to afford 3-(1-methyl-6-(4-(piperazin-1-ylmethyl)piperidin-1-yl)-1H-indazol-3-yl)piperidine-2,6-dione (HCl salt)(0.40 g) as a brown solid.

LC-MS (ESI): m/z=425.40 [M+H]⁺.

### Preparation of 2-((6-((5-chloro-2-(4-((1-(3-(2,6-dioxopiperidin-3-yl)-1-methyl-1H-indazol-6-yl)piperidin-4-yl)methyl)piperazin-1-yl)pyrimidin-4-yl)amino)-1-methyl-2-oxo-1,2-dihydroquinolin-3-yl)oxy)-N-methylacetamide

To a stirred solution 3-(1-methyl-6-(4-(piperazin-1-ylmethyl)piperidin-1-yl)-1H-indazol-3-yl)piperidine-2,6-dione.HCl (0.20 g, 0.47 mmol) and 2-((6-((2,5-dichloropyrimidin-4-yl)amino)-1-methyl-2-oxo-1,2-dihydroquinolin-3-yl)oxy)-N-methylacetamide (0.13 g, 0.32 mmol) in DMSO (2 mL), DIPEA (0.65 mL, 3.76 mmol) was added. The reaction was heated at 100° C. for 3 hours. The reaction mixture was quenched with ice-cold water. The resulting precipitated solid was filtered and dried to obtain crude product. The crude product was purified by preparative HPLC to afford 2-((6-((5-chloro-2-(4-((1-(3-(2,6-dioxopiperidin-3-yl)-1-methyl-1H-indazol-6-yl)piperidin-4-yl)methyl)piperazin-1-yl)pyrimidin-4-yl)amino)-1-methyl-2-oxo-1,2-dihydroquinolin-3-yl)oxy)-N-methylacetamide (0.042 g).

LC-MS (ESI): m/z=794.28 [M−H]⁺.

¹H NMR (400 MHZ, DMSO-d₆): δ 10.84 (s, 1H), 8.87 (s, 1H), 8.05 (s, 1H), 7.96-7.93 (m, 2H), 7.74 (dd, J=2.40, 9.20 Hz, 1H), 7.47 (d, J=9.20 Hz, 2H), 7.12 (s, 1H), 6.91-6.88 (m, 1H), 6.82 (s, 1H), 4.58 (s, 2H), 4.26-4.22 (m, 1H), 3.88 (s, 3H), 3.79-3.76 (m, 2H), 3.67-3.64 (m, 7H), 2.73-2.60 (m, 7H), 2.32 (br s, 4H), 2.30-2.20 (m, 4H), 1.84-1.81 (m, 3H), 1.27-1.24 (m, 2H).

### Example 3. Synthesis of 2-((6-((5-chloro-2-(4-((2-(3-(2,6-dioxopiperidin-3-yl)-1-methyl-1H-indazol-6-yl)-2-azaspiro[3.5]nonan-7-yl)methyl)piperazin-1-yl)pyrimidin-4-yl)amino)-1-methyl-2-oxo-1,2-dihydroquinolin-3-yl)oxy)-N-methylacetamide (Compound 103)

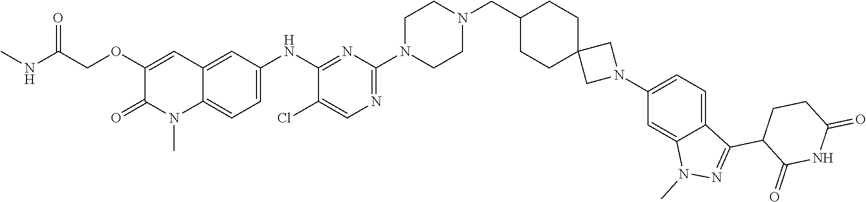 [SMILES: CNC(=O)COc1cc2cc(Nc3nc(N4CCN(CC5CCC6(CC5)CN(c5ccc7c(C8CCC(=O)NC8=O)nn(C)c7c5)C6)CC4)ncc3Cl)ccc2n(C)c1=O]

### Preparation of benzyl 4-((2-(3-(2,6-bis(benzyloxy)pyridin-3-yl)-1-methyl-1H-indazol-6-yl)-2-azaspiro[3.5]nonan-7-yl)methyl)piperazine-1-carboxylate

To a stirred solution of benzyl 4-((2-azaspiro[3.5]nonan-7-yl)methyl)piperazine-1-carboxylate (0.50 g, 1.39 mmol) in 1,4-dioxane:DMF (1:1) was added 3-(2,6-bis(benzyloxy)pyridin-3-yl)-6-bromo-1-methyl-1H-indazole (0.70 g, 1.39 mmol) and Cs₂CO₃ (1.36 g, 4.19 mmol). The reaction was degassed with argon for 5 minutes, then RuPhos (0.13 g, 0.28 mmol) and RuPhos PdG3 (0.058 g, 0.07 mmol) were added to the reaction, and the reaction was heated to 110° C. for 3 hours. The reaction mixture was poured into ice-cold water (10 mL) and extracted with ethyl acetate (2× 50 mL). The organic layers were dried over anhydrous Na₂SO₄, filtered, and dried under vacuum to obtain crude product. The crude product was purified by flash column chromatography (SiO₂, 100-200, 46% ethyl acetate in petroleum ether) to afford benzyl 4-((2-(3-(2,6-bis(benzyloxy)pyridin-3-yl)-1-methyl-1H-indazol-6-yl)-2-azaspiro[3.5]nonan-7-yl)methyl)piperazine-1-carboxylate as a pale brown semi-solid (469 mg).

LC-MS (ESI): m/z=777.42 [M+H]⁺.

### Preparation of 3-(1-methyl-6-(7-(piperazin-1-ylmethyl)-2-azaspiro[3.5]nonan-2-yl)-1H-indazol-3-yl)piperidine-2,6-dione

To a stirred solution of benzyl 4-((2-(3-(2,6-bis(benzyloxy)pyridin-3-yl)-1-methyl-1H-indazol-6-yl)-2-azaspiro[3.5]nonan-7-yl)methyl)piperazine-1-carboxylate (0.32 g, 0.41 mmol) in THE, MeOH, acetic acid, DMF, and 20% Pd(OH)₂ on carbon (0.58 g, 4.18 mmol) was added under nitrogen atmosphere. The resultant reaction mixture was stirred at room temperature under hydrogen atmosphere with balloon pressure for 20 hours. The reaction mixture was diluted with DCM (20 mL) and filtered through a pad of celite and washed with excess of 30% THF in DCM (600 mL). The filtrate was collected and concentrated to afford 3-(1-methyl-6-(7-(piperazin-1-ylmethyl)-2-azaspiro[3.5]nonan-2-yl)-1H-indazol-3-yl)piperidine-2,6-dione as a pale brown semi-solid (166 mg).

LC-MS (ESI): m/z=465.57 [M+H]⁺.

### Preparation of 2-((6-((5-chloro-2-(4-((2-(3-(2,6-dioxopiperidin-3-yl)-1-methyl-1H-indazol-6-yl)-2-azaspiro[3.5]nonan-7-yl)methyl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-6-(7-(piperazin-1-ylmethyl)-2-azaspiro[3.5]nonan-2-yl)-1H-indazol-3-yl)piperidine-2,6-dione (0.16 g, 0.35 mmol) in DMSO (3 mL), 2-((6-((2,5-dichloropyrimidin-4-yl)amino)-1-methyl-2-oxo-1,2-dihydroquinolin-3-yl)oxy)-N-methylacetamide (0.10 g, 0.25 mmol) and DIPEA (0.5 mL, 2.85 mmol) was added. The reaction was heated to 100° C. for 4 hours. The reaction mixture was added to ice-cold water (10 mL) and solid was precipitated out. The solid was filtered and dried under vacuum to obtain crude product. The crude product was purified by preparative HPLC to afford 2-((6-((5-chloro-2-(4-((2-(3-(2,6-dioxopiperidin-3-yl)-1-methyl-1H-indazol-6-yl)-2-azaspiro[3.5]nonan-7-yl)methyl)piperazin-1-yl)pyrimidin-4-yl)amino)-1-methyl-2-oxo-1,2-dihydroquinolin-3-yl)oxy)-N-methylacetamide as an off-white solid (31 mg).

LC-MS (ESI): m/z=836.66 [M+H]⁺.

¹H NMR (400 MHZ, DMSO-d₆): δ 10.95 (s, 1H), 8.86 (s, 1H), 8.05 (s, 1H), 7.96 (s, 1H), 7.94 (d, J=2.40 Hz, 1H), 7.75-7.72 (m, 1H), 7.49-7.45 (m, 2H), 7.12 (s, 1H), 6.36-6.34 (m, 1H), 6.30 (s, 1H), 4.61 (s, 2H), 4.25-4.21 (m, 1H), 3.83 (s, 3H), 3.68 (s, 3H), 3.63-3.55 (m, 6H), 2.67 (d, J=4.40 Hz, 3H), 2.63-2.60 (m, 2H), 2.59-2.54 (m, 1H), 2.36-2.33 (m, 5H), 2.17-2.11 (m, 3H), 1.89 (d, J=12.00 Hz, 2H), 1.73 (d, J=11.20 Hz, 2H), 1.53-1.47 (m, 3H), 1.24 (s, 1H), 0.95 (d, J=12.00 Hz, 2H).

### Example 4. Synthesis of 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-methyl-2-oxo-1,2-dihydroquinolin-3-yl)oxy)-N-methylacetamide (Compound 104)

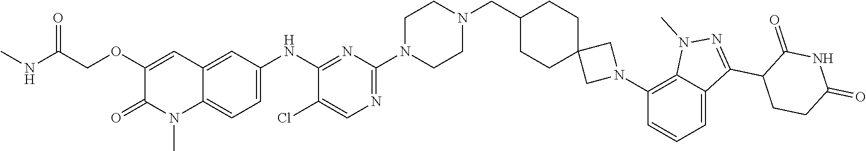 [SMILES: CNC(=O)COc1cc2cc(Nc3nc(N4CCN(CC5CCC6(CC5)CN(c5cccc7c(C8CCC(=O)NC8=O)nn(C)c57)C6)CC4)ncc3Cl)ccc2n(C)c1=O]

### Preparation of benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate

To a stirred solution of benzyl 4-((2-azaspiro[3.5]nonan-7-yl)methyl)piperazine-1-carboxylate (0.31 g, 0.84 mmol) in 1,4-dioxane (3 mL), 3-(2,6-bis(benzyloxy)pyridin-3-yl)-7-bromo-1-methyl-1H-indazole (0.42 g, 0.84 mmol) was added. After degassing with argon for 10 minutes, Cs₂CO₃ (0.82 g, 2.52 mmol) and Pd-PEPPSI-iHept Cl (0.041 g, 0.04 mmol) were added. The reaction was heated at 100° C. for 5 hours. The reaction mixture was quenched with ice-cold water and extracted into ethyl acetate. The combined organic layers were washed with brine solution, dried over anhydrous Na₂SO₄, filtered, and concentrated under vacuum to afford crude product. The crude product was purified by column chromatography (SiO₂; 46% EtOAc in petroleum ether) to afford benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (0.25 g) as a pale brown semi-solid.

LC-MS (ESI): m/z=777.78 [M+H]⁺.

### Preparation of 3-(1-methyl-7-(7-(piperazin-1-ylmethyl)-2-azaspiro[3.5]nonan-2-yl)-1H-indazol-3-yl)piperidine-2,6-dione

To a stirred solution of benzyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (0.32 g, 0.41 mmol) in THF (9 mL), acetic acid (0.3 mL), DMF (0.3 mL), and 20% Pd(OH)₂/C 50% wet basis (0.86 g, 6.15 mmol) were added. The reaction was put under hydrogen atmosphere with balloon pressure at room temperature for 20 hours. The reaction mixture was diluted with DCM and filtered through a pad of celite and washed with excess of 40% THF in DCM. The filtrate was concentrated under reduced pressure to afford 3-(1-methyl-7-(7-(piperazin-1-ylmethyl)-2-azaspiro[3.5]nonan-2-yl)-1H-indazol-3-yl)piperidine-2,6-dione (0.18 g) as a pale brown semi-solid.

LC-MS (ESI): m/z=465.57 [M+H]⁺.

### Preparation of 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-7-(7-(piperazin-1-ylmethyl)-2-azaspiro[3.5]nonan-2-yl)-1H-indazol-3-yl)piperidine-2,6-dione (0.18 g, 0.40 mmol) in DMSO (1.9 mL), 2-((6-((2,5-dichloropyrimidin-4-yl)amino)-1-methyl-2-oxo-1,2-dihydroquinolin-3-yl)oxy)-N-methylacetamide (0.11 g, 0.28 mmol) and DIPEA (0.56 mL, 3.25 mmol) were added. The reaction was heated to 100° C. for 5 hours. The reaction mixture was quenched with ice-cold water (3 mL). The solid precipitated was filtered and dried under vacuum to obtain crude product. The crude product was purified by preparative HPLC to afford 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-methyl-2-oxo-1,2-dihydroquinolin-3-yl)oxy)-N-methylacetamide (0.057 g) as an off-white solid.

LC-MS (ESI): m/z=836.55 [M+H]⁺.

¹H NMR (400 MHZ, DMSO-d₆): δ 10.91 (s, 1H), 8.86 (s, 1H), 8.05 (s, 1H), 7.96-7.93 (m, 2H), 7.74 (dd, J=11.60 Hz, 1H), 7.47 (d, J=9.20 Hz, 1H), 7.20 (d, J=8.00 Hz, 1H), 7.12 (s, 1H), 7.00-6.96 (m, 1H), 6.72 (d, J=7.60 Hz, 1H), 4.62 (s, 2H), 4.32-4.29 (m, 1H), 4.17 (s, 3H), 3.68 (s, 3H), 3.62-3.54 (m, 10H), 2.66 (d, J=4.80 Hz, 5H), 2.50-2.32 (m, 5H), 2.18-2.11 (m, 3H), 1.96 (d, J=12.40 Hz, 2H), 1.73 (d, J=11.60 Hz, 2H), 1.54-1.47 (m, 3H), 0.99-0.96 (m, 2H).

### Example 5. Synthesis of N-((1-(5-chloro-4-((1-methyl-3-(2-(methylamino)-2-oxoethoxy)-2-oxo-1,2-dihydroquinolin-6-yl)amino)pyrimidin-2-yl)piperidin-4-yl)methyl)-3-(2,6-dioxopiperidin-3-yl)benzamide (Compound 105)

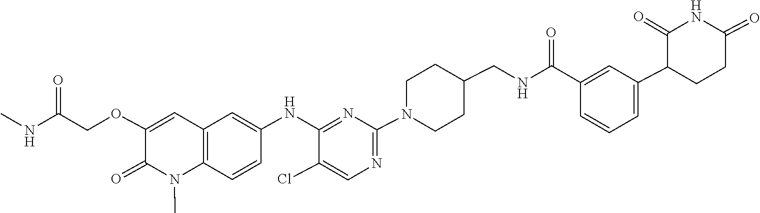 [SMILES: CNC(=O)COc1cc2cc(Nc3nc(N4CCC(CNC(=O)c5cccc(C6CCC(=O)NC6=O)c5)CC4)ncc3Cl)ccc2n(C)c1=O]

### Preparation of tert-butyl 4-((3-(2,6-dioxopiperidin-3-yl)benzamido)methyl)piperidine-1-carboxylate

To a stirred solution of 3-(2,6-dioxopiperidin-3-yl)benzoic acid (0.45 g, 1.92 mmol) in THF (9 mL), tert-butyl 4-(amino methyl)piperidine-1-carboxylate (0.45 g, 2.122 mmol), propanephosphonic acid anhydride (2.45 g, 7.71 mmol), and triethyl amine (0.80 mL, 5.78 mmol) were added. The reaction was stirred at room temperature for 2 hours. The reaction mixture was quenched with water and extracted into ethyl acetate. The separated organic layers were washed with brine solution, dried over anhydrous Na₂SO₄, filtered, and dried under vacuum to isolate crude product. The crude product was purified by flash column chromatography (SiO₂, 100-200, 64% ethyl acetate in petroleum ether) to afford tert-butyl 4-((3-(2,6-dioxopiperidin-3-yl)benzamido)methyl)piperidine-1-carboxylate (0.8 g) as an off white solid.

LC-MS (ESI): m/z=428.43 [M+H]⁺.

### Preparation of 3-(2,6-dioxopiperidin-3-yl)-N-(piperidin-4-ylmethyl)benzamide

To a stirred solution of tert-butyl 4-((3-(2,6-dioxopiperidin-3-yl)benzamido)methyl)piperidine-1-carboxylate (0.8 g, 1.86 mmol) in DCM (4 mL) was added 4 M HCl in dioxane (4 mL) at 0° C. The reaction was allowed to warm to room temperature over 4 hours. The reaction mixture was concentrated under vacuum to afford 3-(2,6-dioxopiperidin-3-yl)-N-(piperidin-4-ylmethyl)benzamide (0.65 g) as an off-white semi-solid.

LC-MS (ESI): m/z=330.37 [M+H]⁺.

### Preparation of N-((1-(5-chloro-4-((1-methyl-3-(2-(methylamino)-2-oxoethoxy)-2-oxo-1,2-dihydroquinolin-6-yl)amino)pyrimidin-2-yl)piperidin-4-yl)methyl)-3-(2,6-dioxopiperidin-3-yl)benzamide

To a stirred solution of 3-(2,6-dioxopiperidin-3-yl)-N-(piperidin-4-ylmethyl)benzamide (0.45 g, 1.36 mmol) in DMSO (4.5 mL), 2-((6-((2,5-dichloropyrimidin-4-yl)amino)-1-methyl-2-oxo-1,2-dihydroquinolin-3-yl)oxy)-N-methylacetamide (0.33 g, 0.82 mmol) and DIPEA (1.9 mL, 10.92 mmol) were added. The reaction was heated at 80° C. for 4 hours. The reaction mixture was quenched with ice-cold water. The precipitated solid was filtered and dried under vacuum to afford crude product. The crude product was purified by preparative HPLC to afford N-((1-(5-chloro-4-((1-methyl-3-(2-(methylamino)-2-oxoethoxy)-2-oxo-1,2-dihydroquinolin-6-yl)amino)pyrimidin-2-yl)piperidin-4-yl)methyl)-3-(2,6-dioxopiperidin-3-yl)benzamide (0.15 g).

LC-MS (ESI): m/z=701.42 [M+H]⁺.

¹H NMR (400 MHZ, DMSO-d₆): δ 10.88 (s, 1H), 8.82 (s, 1H), 8.51-8.48 (m, 1H), 8.04 (s, 1H), 7.95 (d, J=4.40 Hz, 1H), 7.91 (d, J=2.40 Hz, 1H), 7.78-7.74 (m, 2H), 7.70 (s, 1H), 7.47 (d, J=9.20 Hz, 1H), 7.43-7.36 (m, 2H), 7.10 (s, 1H), 4.50 (s, 2H), 4.47-4.47 (m, 2H), 3.94-3.90 (m, 1H), 3.32 (s, 3H), 3.18-3.15 (m, 2H), 2.74-2.63 (m, 6H), 2.22-2.19 (m, 1H), 2.07-2.02 (m, 2H), 1.84-1.71 (m, 3H), 1.15-1.06 (m, 2H).

### Example 6. Synthesis of 2-((6-((5-chloro-2-((3R)-3-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (Compound 106a)

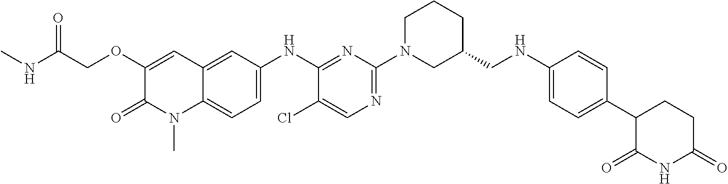 [SMILES: CNC(=O)COc1cc2cc(Nc3nc(N4CCC[C@H](CNc5ccc(C6CCC(=O)NC6=O)cc5)C4)ncc3Cl)ccc2n(C)c1=O]

### Preparation of tert-butyl (R)-3-(((4-(2,6-bis(benzyloxy)pyridin-3-yl)phenyl)amino)methyl)piperidine-1-carboxylate

To a stirred solution of 2,6-bis(benzyloxy)-3-(4-bromophenyl)pyridine (0.70 g, 1.56 mmol) in 1,4-dioxane (14 mL) was added tert-butyl (R)-3-(aminomethyl)piperidine-1-carboxylate (0.37 g, 1.72 mmol). The reaction was purged with argon for 15 minutes, then potassium tert-butoxide (0.52 g, 4.70 mmol) and Brettphos Pd G3 (0.08 g, 0.08 mmol) were added. The reaction was heated to 80° C. for 3 hours. The reaction mixture was quenched with water and extracted in ethyl acetate. The combined organic layers were washed with brine, dried over anhydrous Na₂SO₄, filtered, and concentrated under vacuum to get crude product. The crude product was purified by flash column chromatography (SiO₂, 230-400, 15% ethyl acetate in petroleum ether) to afford tert-butyl (R)-3-(((4-(2,6-bis(benzyloxy)pyridin-3-yl)phenyl)amino)methyl)piperidine-1-carboxylate (0.65 g) as a pale brown semi-solid.

LC-MS (ESI): m/z=580.68 [M+H]⁺.

### Preparation of tert-butyl (3R)-3-(((4-(2,6-dioxopiperidin-3-yl)phenyl)amino)methyl)piperidine-1-carboxylate

To a stirred solution of tert-butyl (R)-3-(((4-(2,6-bis(benzyloxy)pyridin-3-yl)phenyl)amino)methyl)piperidine-1-carboxylate (0.67 g, 1.16 mmol) in THF (20 mL), acetic acid (0.7 mL) and DMF (0.7 mL), and 20% Pd(OH)₂ on carbon 50% wet (0.98 g, 6.98 mmol) was added. The reaction was put under hydrogen atmosphere (balloon pressure) and stirred at room temperature for 16 hours. The reaction mixture was diluted with DCM and filtered through a celite bed. The celite was washed with 30% THF in DCM. The filtrate was concentrated under vacuum to afford tert-butyl (3R)-3-(((4-(2,6-dioxopiperidin-3-yl)phenyl)amino)methyl)piperidine-1-carboxylate (0.353 g) as an off-white solid.

LC-MS (ESI): m/z=402.46 [M+H]⁺.

### Preparation of 3-(4-((((S)-piperidin-3-yl)methyl)amino)phenyl)piperidine-2,6-dione

To a stirred solution of tert-butyl (3R)-3-(((4-(2,6-dioxopiperidin-3-yl)phenyl)amino)methyl)piperidine-1-carboxylate (0.35 g, 0.87 mmol) in DCM (3.5 mL) was added 4 M HCl in dioxane (3.5 mL). The reaction was stirred at room temperature for 2 hours. The reaction mixture was concentrated under vacuum to afford crude 3-(4-((((S)-piperidin-3-yl)methyl)amino)phenyl)piperidine-2,6-dione (0.259 g) as an off-white solid.

LC-MS (ESI): m/z=302.34 [M+H]⁺.

### Preparation of 2-((6-((5-chloro-2-((3R)-3-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide

To a stirred solution 3-(4-((((S)-piperidin-3-yl)methyl)amino)phenyl)piperidine-2,6-dione (0.20 g, 0.66 mmol) in DMSO (2 mL), 2-((6-((2,5-dichloropyrimidin-4-yl)amino)-1-methyl-2-oxo-1,2-dihydroquinolin-3-yl)oxy)-N-methylacetamide (0.135 g, 0.33 mmol) and DIPEA (0.92 mL, 5.31 mmol) were added. The resultant reaction mixture was stirred at 100° C. for 6 hours. The reaction mixture was added to ice-cold water. The resulting solid precipitate was filtered and dried under vacuum to afford crude product. The crude product was purified by preparative HPLC to afford 2-((6-((5-chloro-2-((3R)-3-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (0.034 g) as a light brown solid.

LC-MS (ESI): m/z=673.44 [M+H]⁺.

¹H NMR (400 MHZ, DMSO-d₆): δ 10.75 (s, 1H), 8.82 (s, 1H), 8.03 (s, 1H), 7.93-7.81 (m, 3H), 7.43 (d, J=9.20 Hz, 1H), 7.13 (s, 1H), 6.83 (d, J=6.80 Hz, 2H), 6.45 (s, 2H), 5.57 (s, 1H), 4.52-4.47 (m, 3H), 4.33-4.30 (m, 1H), 3.62-3.59 (m, 4H), 2.97-2.74 (m, 3H), 2.74 (t, J=12.40 Hz, 1H), 2.64 (d, J=5.20 Hz, 3H), 2.59-2.51 (m, 2H), 2.07-1.87 (m, 3H), 1.75-1.66 (m, 2H), 1.40-1.37 (m, 1H), 1.26-1.23 (m, 1H).

### Example 7. Synthesis of 2-((6-((5-chloro-2-((3S)-3-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (Compound 106b)

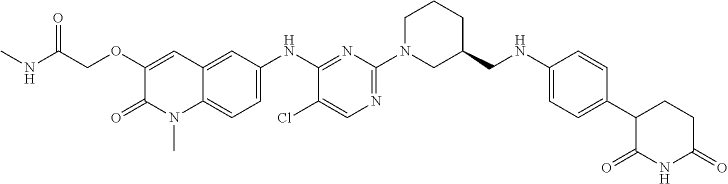 [SMILES: CNC(=O)COc1cc2cc(Nc3nc(N4CCC[C@@H](CNc5ccc(C6CCC(=O)NC6=O)cc5)C4)ncc3Cl)ccc2n(C)c1=O]

### Preparation of tert-butyl(S)-3-(((4-(2,6-bis(benzyloxy)pyridin-3-yl)phenyl)amino)methyl)piperidine-1-carboxylate

To a stirred solution of 2,6-bis(benzyloxy)-3-(4-bromophenyl)pyridine (0.71 g, 1.59 mmol, tert-butyl(S)-3-(aminomethyl)piperidine-1-carboxylate (0.41 g, 1.91 mmol) in 1,4-dioxane (14.0 mL) was added potassium tert-butoxide (0.53 g, 4.77 mmol). The reaction was degassed with argon for 10 minutes, then BrettPhos Pd G3 (0.072 g, 0.08 mmol) was added, and the reaction was again degassed for 5 minutes. The reaction mixture was stirred at 80° C. for 4 hours. The reaction mixture was cooled to room temperature, filtered through a celite bed, and the filtrate was concentrated. The crude reaction mixture was purified by flash column chromatography (SiO₂, 230-400, 10% ethyl acetate in petroleum ether) to afford tert-butyl(S)-3-(((4-(2,6-bis(benzyloxy)pyridin-3-yl)phenyl)amino)methyl)piperidine-1-carboxylate (0.64 g) as a pale yellow solid.

LC-MS (ESI): m/z=580.65 [M+H]⁺.

### Preparation of tert-butyl (3S)-3-(((4-(2,6-dioxopiperidin-3-yl)phenyl)amino)methyl)piperidine-1-carboxylate

To a stirred solution of tert-butyl(S)-3-(((4-(2,6-bis(benzyloxy)pyridin-3-yl)phenyl)amino)methyl)piperidine-1-carboxylate (0.6 g, 1.03 mmol) in THF (18 mL) was added acetic acid (0.6 mL). The reaction was degassed with nitrogen for 5 minutes. Palladium hydroxide on carbon (0.9 g) was added. The reaction mixture was put under hydrogen atmosphere (15 psi) and stirred at room temperature for 7 hours. The reaction mixture was filtered through a celite bed and washed with THF:DCM (1:1). The filtrate was concentrated and dried to isolate tert-butyl (3S)-3-(((4-(2,6-dioxopiperidin-3-yl)phenyl)amino)methyl)piperidine-1-carboxylate as a black solid (0.43 g).

LC-MS (ESI): m/z=402.46 [M+H]⁺.

### Preparation of 3-(4-((((S)-piperidin-3-yl)methyl)amino)phenyl)piperidine-2,6-dione

A stirred solution of tert-butyl (3S)-3-(((4-(2,6-dioxopiperidin-3-yl)phenyl)amino)methyl)piperidine-1-carboxylate (0.4 g, 0.99 mmol) in DCM (4 mL) was cooled to 0° C. TFA (4 mL) was added and the resulting reaction mixture was stirred at room temperature for 3 hours. The reaction mixture was concentrated. The crude product was co-evaporated with toluene and triturated with diethyl ether to afford 3-(4-((((S)-piperidin-3-yl)methyl)amino)phenyl)piperidine-2,6-dione (300 mg, TFA salt) a brown solid.

LC-MS (ESI): m/z=302.37 [M+H]⁺.

### Preparation of 2-((6-((5-chloro-2-((3S)-3-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(4-((((S)-piperidin-3-yl)methyl)amino)phenyl)piperidine-2,6-dione (0.3 g, 0.99 mmol) and 2-((6-((2,5-dichloropyrimidin-4-yl)amino)-1-methyl-2-oxo-1,2-dihydroquinolin-3-yl)oxy)-N-methylacetamide (0.12 g, 0.39 mmol) in DMSO was added N,N-diisopropylethylamine (1.39 mL, 7.96 mmol). The reaction mixture was stirred at 100° C. for 6 hours. The reaction mixture was cooled to room temperature and poured into ice water. The resulting precipitate was filtered and dried to isolate crude product. The crude product was purified by prep-HPLC to afford 2-((6-((5-chloro-2-((3S)-3-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (70 mg) as a pale brown solid.

LC-MS (ESI): m/z=673.51 [M+H]⁺.

¹H NMR (400 MHZ, DMSO-d₆): δ 10.75 (s, 1H), 8.81 (s, 1H), 8.03 (s, 1H), 7.93-7.81 (m, 2H), 7.43 (d, J=9.20 Hz, 1H), 7.13 (s, 1H), 6.83 (d, J=6.80 Hz, 2H), 6.45 (s, 2H), 5.57 (s, 1H), 4.54-4.49 (s, 3H), 4.31 (d, J=12.80 Hz, 1H), 3.62-3.60 (m, 4H), 2.90-2.89 (m, 3H), 2.74 (t, J=12.40 Hz, 1H), 2.71-2.63 (m, 4H), 2.42-2.41 (m, 1H), 2.07-1.66 (m, 5H), 1.40-1.23 (m, 2H).

### Example 8. Synthesis of 2-((6-((5-chloro-2-((2R)-2-(((3-(2,6-dioxopiperidin-3-yl)phenyl)amino)methyl)pyrrolidin-1-yl)pyrimidin-4-yl)amino)-1-methyl-2-oxo-1,2-dihydroquinolin-3-yl)oxy)-N-methylacetamide (Compound 260a)

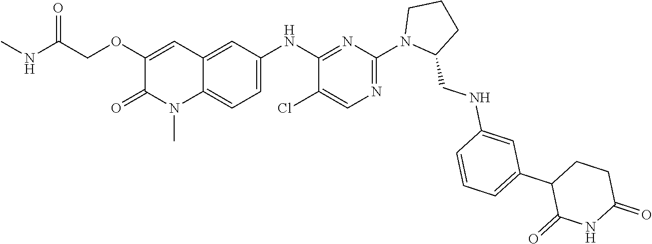 [SMILES: CNC(=O)COc1cc2cc(Nc3nc(N4CCC[C@@H]4CNc4cccc(C5CCC(=O)NC5=O)c4)ncc3Cl)ccc2n(C)c1=O]

### Preparation of tert-butyl (R)-2-(((3-(2,6-bis(benzyloxy)pyridin-3-yl)phenyl)amino)methyl)pyrrolidine-1-carboxylate

To a stirred solution of 2,6-bis(benzyloxy)-3-(3-bromophenyl)pyridine (0.75 g, 1.68 mmol) in 1,4-dioxane (1.4 mL) was added tert-butyl (R)-2-(aminomethyl)pyrrolidine-1-carboxylate (0.369 g, 1.848 mmol). The reaction was purged with argon for 15 minutes, then potassium tert-butoxide (0.564 g, 5.041 mmol) and BrettPhos Pd G3 (0.076 g, 0.084 mmol) were added, and the reaction was heated to 80° C. for 3 hours. The reaction mixture was quenched with water and extracted in ethyl acetate. The combined organic layers were washed with brine solution, dried over anhydrous Na₂SO₄, filtered, and concentrated under vacuum. The crude reaction mixture was purified by flash column chromatography (SiO₂, 230-400, 15% ethyl acetate in petroleum ether) to afford tert-butyl (R)-2-(((3-(2,6-bis(benzyloxy)pyridin-3-yl)phenyl)amino)methyl)pyrrolidine-1-carboxylate (0.600 g) as a yellow semi-solid.

LC-MS (ESI): m/z=566.67 [M+H]⁺.

### Preparation of tert-butyl (2R)-2-(((3-(2,6-dioxopiperidin-3-yl)phenyl)amino)methyl)pyrrolidine-1-carboxylate

To a stirred solution of tert-butyl (R)-2-(((3-(2,6-bis(benzyloxy)pyridin-3-yl)phenyl)amino)methyl)pyrrolidine-1-carboxylate (0.60 g, 1.061 mmol) in THF (10 mL) was added acetic acid (0.5 mL), DMF (0.5 mL), and 20% Pd(OH)₂ on carbon (50% wet, 0.945 g, 6.72 mmol). The reaction mixture was stirred under hydrogen atmosphere (balloon pressure) at room temperature for 16 hours. The reaction filtered through celite bed and washed with THF. The filtrate was concentrated under vacuum to afford tert-butyl (2R)-2-(((3-(2,6-dioxopiperidin-3-yl)phenyl)amino)methyl)pyrrolidine-1-carboxylate (0.40 g).

LC-MS (ESI): m/z=388.41 [M+H]⁺.

### Preparation of 3-(3-((((R)-pyrrolidin-2-yl)methyl)amino)phenyl)piperidine-2,6-dione hydrochloride

To a stirred solution of tert-butyl (2R)-2-(((3-(2,6-dioxopiperidin-3-yl)phenyl)amino)methyl)pyrrolidine-1-carboxylate (0.4 g, 1.032 mmol) in DCM (4.0 mL) was added 4 M HCl in 1,4-dioxane (4.0 mL) and the reaction mixture was stirred at room temperature for 2 hours. The reaction mixture was concentrated under vacuum. The crude reaction was purified by trituration with n-pentane to afford 3-(3-((((R)-pyrrolidin-2-yl)methyl)amino)phenyl)piperidine-2,6-dione hydrochloride (0.28 g) as a brown solid.

LC-MS (ESI): m/z=288.32 [M+H]⁺.

### Preparation of 2-((6-((5-chloro-2-((2R)-2-(((3-(2,6-dioxopiperidin-3-yl)phenyl)amino)methyl)pyrrolidin-1-yl)pyrimidin-4-yl)amino)-1-methyl-2-oxo-1,2-dihydroquinolin-3-yl)oxy)-N-methylacetamide

To a stirred solution of 3-(3-((((R)-pyrrolidin-2-yl)methyl)amino)phenyl)piperidine-2,6-dione hydrochloride (0.25 g, 0.73 mmol) in DMSO (4.2 mL), 2-((6-((2,5-dichloropyrimidin-4-yl)amino)-1-methyl-2-oxo-1,2-dihydroquinolin-3-yl)oxy)-N-methylacetamide (0.11 g, 0.29 mmol) and DIPEA (1.53 mL, 8.77 mmol) were added. The resulting reaction mixture was heated to 100° C. for 6 hours. The reaction was quenched with ice-cold water. The resultant precipitate was filtered and dried under vacuum to afford crude product. The crude product was purified by preparative HPLC to afford 2-((6-((5-chloro-2-((2R)-2-(((3-(2,6-dioxopiperidin-3-yl)phenyl)amino)methyl) 205pyrrolidine-1-yl)pyrimidin-4-yl)amino)-1-methyl-2-oxo-1,2-dihydroquinolin-3-yl)oxy)-N-methylacetamide (0.053 g) as an off-white solid.

LC-MS (ESI): m/z=659.41 [M+H]⁺.

¹H NMR (400 MHZ, DMSO-d₆): δ 10.78 (s, 1H), 8.79 (s, 1H), 8.43 (s, 1H), 7.45-8.11 (m, 4H), 7.14 (s, 2H), 6.81-6.31 (m, 3H), 6.30-5.47 (m, 1H), 4.52 (s, 2H), 4.23-4.23 (m, 1H), 3.67-3.31 (m, 7H), 2.96 (s, 1H), 1.45 (d, 3H), 1.27 (d, 1H), 0.00 (t, J=500.2 Hz, 1H), 2.47 (m, 6H).

### Example 9. Synthesis of 2-((6-((5-chloro-2-((2S)-2-(((3-(2,6-dioxopiperidin-3-yl)phenyl)amino)methyl)pyrrolidin-1-yl)pyrimidin-4-yl)amino)-1-methyl-2-oxo-1,2-dihydroquinolin-3-yl)oxy)-N-methylacetamide (Compound 260b)

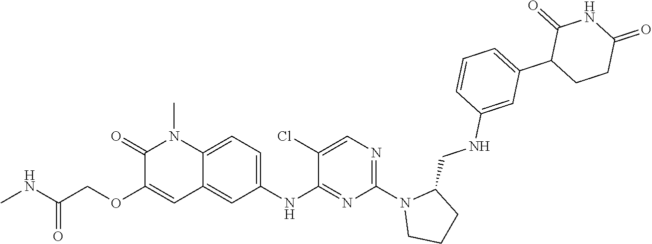 [SMILES: CNC(=O)COc1cc2cc(Nc3nc(N4CCC[C@H]4CNc4cccc(C5CCC(=O)NC5=O)c4)ncc3Cl)ccc2n(C)c1=O]

### Preparation of tert-butyl(S)-2-(((3-(2,6-bis(benzyloxy)pyridin-3-yl)phenyl)amino)methyl)pyrrolidine-1-carboxylate

A solution of 2,6-bis(benzyloxy)-3-(3-bromophenyl)pyridine (0.45 g, 1.01 mmol), tert-butyl (S)-2-(aminomethyl)pyrrolidine-1-carboxylate (0.40 g, 2.02 mmol), and sodium tertiary butoxide (0.29 g, 3.03 mmol) in 1,4-dioxane (9.0 mL) was degassed with argon for 5 minutes. Brettphos Pd G3 (0.04 g, 0.05 mmol) was added, and the reaction was degassed for another 5 minutes. The reaction mixture was heated at 100° C. for 3 hours. The reaction was quenched with ice-cold water and extracted into ethyl acetate. The separated organic layers were washed with brine solution, dried over anhydrous Na₂SO₄, filtered, and dried under vacuum to isolate crude product. The crude product was purified by column chromatography (SiO₂; 24% EtOAc in petroleum ether) to afford tert-butyl(S)-2-(((3-(2,6-bis(benzyloxy)pyridin-3-yl)phenyl)amino)methyl)pyrrolidine-1-carboxylate (500 mg) as an off-white solid.

LC-MS (ESI): m/z=566 [M+H]⁺.

### Preparation of tert-butyl (2S)-2-(((3-(2,6-dioxopiperidin-3-yl)phenyl)amino)methyl)pyrrolidine-1-carboxylate

To a stirred solution of tert-butyl(S)-2-(((3-(2,6-bis(benzyloxy)pyridin-3-yl)phenyl)amino)methyl)pyrrolidine-1-carboxylate (0.50 g, 0.88 mmol) in THF (15.0 mL), DMF (3.0 mL), 1,4-dioxane (5.0 mL), and acetic acid (0.50 mL) was added. The reaction was degassed with nitrogen for 5 minutes. 20% Palladium hydroxide on carbon, powder (0.5 g) was added. The reaction mixture was stirred under hydrogen atmosphere with balloon pressure at room temperature for 16 hours. The reaction mixture was filtered through a celite bed and washed with THF:DCM (1:1). The filtrate was concentrated to afford crude product. The crude product was triturated with n-pentane and dried to afford tert-butyl (2S)-2-(((3-(2,6-dioxopiperidin-3-yl)phenyl)amino)methyl)pyrrolidine-1-carboxylate (0.35 g) as a brown semi-solid.

LC-MS (ESI): m/z=386.3 [M+H]⁺.

### Preparation of 3-(3-((((S)-pyrrolidin-2-yl)methyl)amino)phenyl)piperidine-2,6-dione

A stirred solution of tert-butyl (2S)-2-(((3-(2,6-dioxopiperidin-3-yl)phenyl)amino)methyl)pyrrolidine-1-carboxylate in DCM was cooled to 0° C. and 4 M HCl in dioxane (3.5 mL) was added. The reaction mixture was stirred at room temperature for 3 hours. The solvent was evaporated to afford crude product. The crude product was triturated with n-pentane to isolate 3-(3-((((S)-pyrrolidin-2-yl)methyl)amino)phenyl)piperidine-2,6-dione (0.25 g) as a yellow, brown solid.

LC-MS (ESI): m/z=288.33 [M+H]⁺.

### Preparation of 2-((6-((5-chloro-2-((2S)-2-(((3-(2,6-dioxopiperidin-3-yl)phenyl)amino)methyl)pyrrolidin-1-yl)pyrimidin-4-yl)amino)-1-methyl-2-oxo-1,2-dihydroquinolin-3-yl)oxy)-N-methylacetamide

To a stirred solution of 3-(3-((((S)-pyrrolidin-2-yl)methyl)amino)phenyl)piperidine-2,6-dione (0.25 g, 0.73 mmol) and 2-((6-((2,5-dichloropyrimidin-4-yl)amino)-1-methyl-2-oxo-1,2-dihydroquinolin-3-yl)oxy)-N-methylacetamide (0.14 g, 0.36 mmol) in DMSO was added N,N-diisopropylethylamine (1.04 mL, 5.84 mmol). The reaction mixture was stirred at 100° C. for 5 hours. The reaction mixture was poured into ice-cold water and stirred for 15 minutes resulting in a precipitate. The precipitate was filtered and dried to afford crude product. The crude product was purified by prep-HPLC to afford 2-((6-((5-chloro-2-((2S)-2-(((3-(2,6-dioxopiperidin-3-yl)phenyl)amino)methyl)pyrrolidin-1-yl)pyrimidin-4-yl)amino)-1-methyl-2-oxo-1,2-dihydroquinolin-3-yl)oxy)-N-methylacetamide (0.079 g) as an off-white solid.

LC-MS (ESI): m/z=659.41 [M+H]⁺.

¹H NMR (400 MHZ, DMSO-d₆): δ 10.77 (s, 1H), 8.79 (s, 1H), 7.79-7.87 (m, 4H), 6.62-6.80 (m, 4H), 6.31-6.33 (m, 2H), 5.93 (s, 1H), 5.48 (s, 1H), 4.55 (d, J=20.40 Hz, 2H), 4.23 (s, 1H), 3.49-3.51 (m, 7H), 2.89 (s, 1H), 2.66 (d, J=4.40 Hz, 4H), 1.93-1.98 (m, 6H), 1.24 (s, 1H).

### Example 10. Synthesis of 2-((6-((5-chloro-2-((3S)-3-(((3-(2,6-dioxopiperidin-3-yl)-1-methyl-1H-indazol-6-yl)amino)methyl)piperidin-1-yl)pyrimidin-4-yl)amino)-1-methyl-2-oxo-1,2-dihydroquinolin-3-yl)oxy)-N-methylacetamide (Compound 261a)

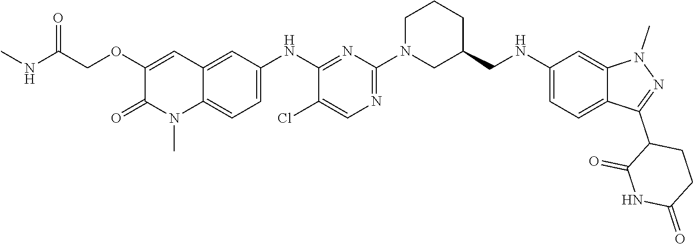 [SMILES: CNC(=O)COc1cc2cc(Nc3nc(N4CCC[C@@H](CNc5ccc6c(C7CCC(=O)NC7=O)nn(C)c6c5)C4)ncc3Cl)ccc2n(C)c1=O]

### Preparation of tert-butyl(S)-3-(((3-(2,6-bis(benzyloxy)pyridin-3-yl)-1-methyl-1H-indazol-6-yl)amino)methyl)piperidine-1-carboxylate

To a stirred solution of 3-(2,6-bis(benzyloxy)pyridin-3-yl)-6-bromo-1-methyl-1H-indazole (0.71 g, 1.42 mmol) and tert-butyl(S)-3-(aminomethyl)piperidine-1-carboxylate (0.36 g, 1.70 mmol) in 1,4-dioxane was added sodium tert-butoxide (0.41 g, 4.26 mmol). The reaction was degassed with argon for 10 minutes, then BrettPhos Pd G3 (0.06 g, 0.07 mmol) was added, and the reaction was degassed for an additional 5 minutes. The reaction mixture was stirred at 80° C. for 4 hours. The reaction mixture was cooled to room temperature, filtered through a celite bed, and the filtrate was concentrated to isolate crude product. The crude product was purified by flash column chromatography (SiO₂, 230-400, 15% Ethyl acetate in Pet ether) to afford tert-butyl(S)-3-(((3-(2,6-bis(benzyloxy)pyridin-3-yl)-1-methyl-1H-indazol-6-yl)amino)methyl)piperidine-1-carboxylate (0.65 g) as a pale yellow solid.

LC-MS (ESI): m/z=634.70 [M+H]⁺.

### Preparation of tert-butyl (3S)-3-(((3-(2,6-dioxopiperidin-3-yl)-1-methyl-1H-indazol-6-yl)amino)methyl)piperidine-1-carboxylate

To a stirred solution of tert-butyl(S)-3-(((3-(2,6-bis(benzyloxy)pyridin-3-yl)-1-methyl-1H-indazol-6-yl)amino)methyl)piperidine-1-carboxylate (0.60 g, 0.95 mmol) in THF was added acetic acid (0.6 mL). The reaction was degassed with nitrogen for 5 minutes, then palladium hydroxide on carbon (1.80 g) was added, and the reaction was put under hydrogen atmosphere (15 psi) at room temperature for 3 hours. The reaction mixture was filtered through a celite bed and washed with THF:DCM (1:1). The filtrate was concentrated and dried to afford tert-butyl (3S)-3-(((3-(2,6-dioxopiperidin-3-yl)-1-methyl-1H-indazol-6-yl)amino)methyl)piperidine-1-carboxylate as a black solid (0.40 g).

LC-MS (ESI): m/z=456.54 [M+H]⁺.

### Preparation of 3-(1-methyl-6-((((R)-piperidin-3-yl)methyl)amino)-1H-indazol-3-yl)piperidine-2,6-dione

A stirred solution of tert-butyl (3S)-3-(((3-(2,6-dioxopiperidin-3-yl)-1-methyl-1H-indazol-6-yl)amino)methyl)piperidine-1-carboxylate (0.4 g, 0.88 mmol) in DCM was cooled to 0° C., and TFA (2 mL) was added dropwise. The resulting reaction mixture was stirred at room temperature for 3 hours. The reaction mixture was concentrated and co-evaporated with toluene to get the crude product. The resultant solid was triturated with diethyl ether and dried to afford 3-(1-methyl-6-((((R)-piperidin-3-yl)methyl)amino)-1H-indazol-3-yl)piperidine-2,6-dione (0.31 g) as a brown solid.

LC-MS (ESI): m/z=356.42 [M+H]⁺.

### Preparation of 2-((6-((5-chloro-2-((3S)-3-(((3-(2,6-dioxopiperidin-3-yl)-1-methyl-1H-indazol-6-yl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-((((R)-piperidin-3-yl)methyl)amino)-1H-indazol-3-yl)piperidine-2,6-dione (0.31 g, 0.87 mmol) and 2-((6-((2,5-dichloropyrimidin-4-yl)amino)-1-methyl-2-oxo-1,2-dihydroquinolin-3-yl)oxy)-N-methylacetamide (0.14 g, 0.35 mmol) in DMSO was added N,N-diisopropylethylamine (1.21 mL, 6.98 mmol). The reaction mixture was stirred at 100° C. for 6 hours. The reaction mixture cooled to room temperature and poured into ice water. The resulting precipitate was filtered and dried to get crude product. The crude product was purified by prep-HPLC to afford 2-((6-((5-chloro-2-((3S)-3-(((3-(2,6-dioxopiperidin-3-yl)-1-methyl-1H-indazol-6-yl)amino)methyl)piperidin-1-yl)pyrimidin-4-yl)amino)-1-methyl-2-oxo-1,2-dihydroquinolin-3-yl)oxy)-N-methylacetamide (40 mg) a pale brown solid.

LC-MS (ESI): m/z=727.5 [M+H]⁺.

¹H NMR (400 MHZ, DMSO-d₆): δ 10.82 (s, 1H), 8.81 (s, 1H), 8.04 (s, 1H), 7.91-7.81 (m, 3H), 7.42 (d, J=9.20 Hz, 1H), 7.27 (d, J=8.80 Hz, 1H), 7.11 (s, 1H), 6.48 (d, J=7.20 Hz, 1H), 6.30 (d, J=Hz, 1H), 5.93 (s, 1H), 4.52 (s, 3H), 4.32 (d, J=12.40 Hz, 1H), 4.17 (q, J=5.20 Hz, 1H), 3.77 (s, 3H), 3.60 (s, 3H), 3.03-2.94 (m, 3H), 2.80 (t, J=10.00 Hz, 1H), 2.65-2.58 (m, 5H), 2.25-2.15 (m, 2H), 1.94-1.69 (m, 3H), 1.31-1.31 (m, 2H).

### Example 11. Synthesis of 2-((6-((5-chloro-2-((3R)-3-(((3-(2,6-dioxopiperidin-3-yl)-1-methyl-1H-indazol-6-yl)amino)methyl)piperidin-1-yl)pyrimidin-4-yl)amino)-1-methyl-2-oxo-1,2-dihydroquinolin-3-yl)oxy)-N-methylacetamide (Compound 261b)

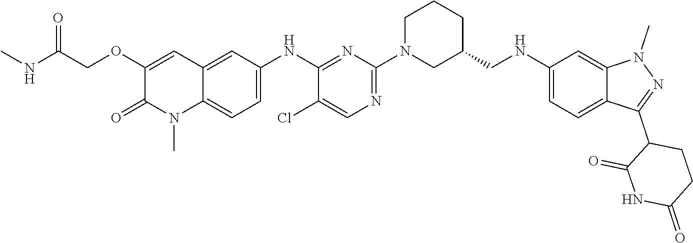 [SMILES: CNC(=O)COc1cc2cc(Nc3nc(N4CCC[C@H](CNc5ccc6c(C7CCC(=O)NC7=O)nn(C)c6c5)C4)ncc3Cl)ccc2n(C)c1=O]

### Preparation of tert-butyl (R)-3-(((3-(2,6-bis(benzyloxy)pyridin-3-yl)-1-methyl-1H-indazol-6-yl)amino)methyl)piperidine-1-carboxylate

To a stirred solution of 3-(2,6-bis(benzyloxy)pyridin-3-yl)-6-bromo-1-methyl-1H-indazole (0.60 g, 1.19 mmol) and tert-butyl (R)-3-(aminomethyl)piperidine-1-carboxylate (0.31 g, 1.43 mmol) in 1,4-dioxane was added sodium tert-butoxide (0.34 g, 3.59 mmol). The reaction was degassed with argon for 10 minutes, then BrettPhos Pd G3 (0.05 g, 0.06 mmol) was added, and the reaction was again degassed for 5 minutes. The reaction mixture was stirred at 80° C. for 4 hours. The reaction mixture was cooled to room temperature and filtered through a celite bed. The filtrate was concentrated to isolate the crude product. The crude product was purified by flash column chromatography (SiO₂, 230-400, 16% ethyl acetate in petroleum ether) to afford tert-butyl (R)-3-(((3-(2,6-bis(benzyloxy)pyridine-3-yl)-1-methyl-1H-indazol-6-yl)amino)methyl)piperidine-1-carboxylate (0.45 g) as a pale yellow solid.

LC-MS (ESI): m/z=634.75 [M+H]⁺.

### Preparation of tert-butyl (3R)-3-(((3-(2,6-dioxopiperidin-3-yl)-1-methyl-1H-indazol-6-yl)amino)methyl)piperidine-1-carboxylate

To a stirred solution of tert-butyl (R)-3-(((3-(2,6-bis(benzyloxy)pyridin-3-yl)-1-methyl-1H-indazol-6-yl)amino)methyl)piperidine-1-carboxylate (0.45 g, 0.71 mmol) in THF was added acetic acid (0.45 mL), and the reaction was degassed with nitrogen for 5 minutes. Palladium hydroxide on carbon (1.35 g) was added, and the reaction mixture was put under hydrogen atmosphere (15 psi) at room temperature for 3 hours. The reaction mixture was filtered through a celite bed and washed with THF:DCM (1:1). The filtrate was concentrated and dried to afford tert-butyl (3R)-3-(((3-(2,6-dioxopiperidin-3-yl)-1-methyl-1H-indazol-6-yl)amino)methyl)piperidine-1-carboxylate as a black solid (0.41 g).

LC-MS (ESI): m/z=456.54 [M+H]⁺.

### Preparation of 3-(1-methyl-6-((((S)-piperidin-3-yl)methyl)amino)-1H-indazol-3-yl)piperidine-2,6-dione

A stirred solution of tert-butyl (3R)-3-(((3-(2,6-dioxopiperidin-3-yl)-1-methyl-1H-indazol-6-yl)amino)methyl)piperidine-1-carboxylate (0.4 g, 0.88 mmol) in DCM was cooled to 0° C., and TFA (2 mL) was added to the reaction dropwise. The resulting reaction mixture was stirred at room temperature for 3 hours. The reaction mixture was concentrated and then co-evaporated with toluene to get crude product. The crude product was triturated with diethyl ether to afford 3-(1-methyl-6-((((S)-piperidin-3-yl)methyl)amino)-1H-indazol-3-yl)piperidine-2,6-dione (0.30 g) as a brown solid.

LC-MS (ESI): m/z=356.42 [M+H]⁺.

### Preparation of 2-((6-((5-chloro-2-((3R)-3-(((3-(2,6-dioxopiperidin-3-yl)-1-methyl-1H-indazol-6-yl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-((((S)-piperidin-3-yl)methyl)amino)-1H-indazol-3-yl)piperidine-2,6-dione (0.30 g, 0.84 mmol) and 2-((6-((2,5-dichloropyrimidin-4-yl)amino)-1-methyl-2-oxo-1,2-dihydroquinolin-3-yl)oxy)-N-methylacetamide (0.13 g, 0.33 mmol) in DMSO was added N,N-diisopropylethylamine (1.8 mL, 6.75 mmol). The reaction mixture was stirred at 100° C. for 6 hours. The reaction mixture was cooled to room temperature, poured into ice water, and stirred for 15 minutes. The resulting precipitate was filtered and dried to isolate crude product. The crude product was purified by prep-HPLC to afford 2-((6-((5-chloro-2-((3R)-3-(((3-(2,6-dioxopiperidin-3-yl)-1-methyl-1H-indazol-6-yl)amino)methyl)piperidin-1-yl)pyrimidin-4-yl)amino)-1-methyl-2-oxo-1,2-dihydroquinolin-3-yl)oxy)-N-methylacetamide (70 mg) as a pale brown solid.

LC-MS (ESI): m/z=727.57 [M+H]⁺.

¹H NMR (400 MHZ, DMSO-d₆): δ 10.82 (s, 1H), 8.81 (s, 1H), 8.04 (s, 1H), 7.90-7.81 (m, 3H), 7.42 (d, J=9.20 Hz, 1H), 7.27 (d, J=8.80 Hz, 1H), 7.11 (s, 1H), 6.48 (d, J=7.20 Hz, 1H), 6.30 (s, 1H), 5.93 (br s, 1H), 4.52 (s, 3H), 4.32 (d, J=12.40 Hz, 1H), 4.17 (q, J=5.20 Hz, 1H), 3.77 (s, 3H), 3.60 (s, 3H), 3.03-2.94 (m, 3H), 2.80 (t, J=10.00 Hz, 1H), 2.67-2.58 (m, 5H), 2.25-2.13 (m, 2H), 1.94-1.69 (m, 3H), 1.44-1.28 (m, 2H).

### Example 12. Synthesis of 2-((6-((5-chloro-2-((((1s,4s)-4-((3-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (Compound 262a)

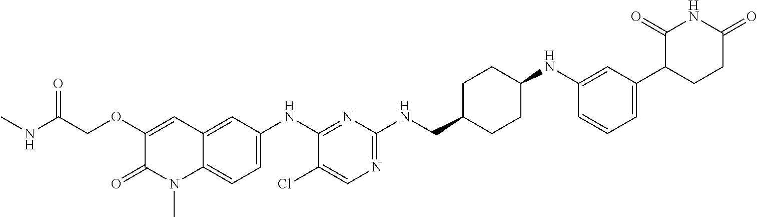 [SMILES: CNC(=O)COc1cc2cc(Nc3nc(NC[C@H]4CC[C@@H](Nc5cccc(C6CCC(=O)NC6=O)c5)CC4)ncc3Cl)ccc2n(C)c1=O]

### Preparation of tert-butyl (((1s,4s)-4-((3-(2,6-bis(benzyloxy)pyridin-3-yl)phenyl)amino)cyclohexyl)methyl) carbamate

A solution of 2,6-bis(benzyloxy)-3-(3-bromophenyl)pyridine (0.75 g, 1.68 mmol), tert-butyl (((1s,4s)-4-aminocyclohexyl)methyl) carbamate (0.77 g, 3.37 mmol), and sodium tertiary butoxide (0.48 g, 5.04 mmol) in 1,4-dioxane (15.0 mL) was degassed with argon for 5 minutes, then Brettphos Pd G3 (0.07 g, 0.08 mmol) was added, and the reaction was degassed for another 5 minutes. The reaction mixture was heated at 100° C. for 3 hours. The reaction was quenched with ice-cold water and extracted into ethyl acetate. The organic layer was washed with brine, dried over anhydrous Na₂SO₄, filtered, and dried under vacuum to get crude product. The crude product was purified by column chromatography (SiO₂; 24% EtOAc in petroleum ether) to afford tert-butyl (((1s,4s)-4-((3-(2,6-bis(benzyloxy)pyridin-3-yl)phenyl)amino)cyclohexyl)methyl) carbamate (500 mg) as off-white solid.

LC-MS (ESI): m/z=593.75 [M+H]⁺.

### Preparation of tert-butyl (((1s,4s)-4-((3-(2,6-dioxopiperidin-3-yl)phenyl)amino)cyclohexyl)methyl) carbamate

To a stirred solution of tert-butyl (((1s,4s)-4-((3-(2,6-bis(benzyloxy)pyridin-3-yl)phenyl)amino)cyclohexyl)methyl) carbamate (0.50 g, 0.84 mmol) in THF (15.00 mL), DMF (3.0 mL), and 1,4-dioxane (5.0 mL) was added acetic acid (0.50 mL). The reaction was degassed with nitrogen for 5 minutes. Palladium hydroxide on carbon, powder (2.00 g) was added, and the reaction mixture was stirred under hydrogen atmosphere with balloon pressure at room temperature for 16 hours. The reaction was filtered through a celite bed and washed with THF:DCM (1:1). The filtrate was concentrated and dried to afford crude product. The crude was triturated with n-pentane to afford tert-butyl (((1s,4s)-4-((3-(2,6-dioxopiperidin-3-yl)phenyl)amino)cyclohexyl)methyl) carbamate (0.40 g) as a brown semi-solid.

LC-MS (ESI): m/z=414 [M−H]⁻.

### Preparation of 3-(3-(((1s,4s)-4-(aminomethyl)cyclohexyl)amino)phenyl)piperidine-2,6-dione

A stirred solution of tert-butyl (((1s,4s)-4-((3-(2,6-dioxopiperidin-3-yl)phenyl)amino)cyclohexyl)methyl) carbamate in DCM was cooled to 0° C., and 4 M HCl in dioxane (4.0 mL) was added to the reaction. The resultant reaction mixture was stirred and allowed to warm to room temperature over 3 hours. The solvent of the reaction mixture was evaporated to isolate crude product. The crude product was triturated with n-pentane to afford 3-(3-(((1s,4s)-4-(aminomethyl)cyclohexyl)amino)phenyl)piperidine-2,6-dione (0.30 g) as a yellow, brown solid.

LC-MS (ESI): m/z=316.39 [M+H]⁺.

### Preparation of 2-((6-((5-chloro-2-((((1s,4s)-4-((3-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide

To a stirred solution of 3-(3-(((1s,4s)-4-(aminomethyl)cyclohexyl)amino)phenyl)piperidine-2,6-dione (0.30 g, 0.95 mmol) and 2-((6-((2,5-dichloropyrimidin-4-yl)amino)-1-methyl-2-oxo-1,2-dihydroquinolin-3-yl)oxy)-N-methylacetamide (0.19 g, 0.47 mmol) in DMSO was added N,N-diisopropylethylamine (1.35 mL, 7.60 mmol). The reaction mixture was stirred at 100° C. for 5 hours in microwave. The reaction was quenched in ice-cold water and stirred for 15 minutes. The resulting precipitate was filtered and dried to isolate crude product. The crude product was purified by prep-HPLC to afford 2-((6-((5-chloro-2-((((1s,4s)-4-((3-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (0.058 g) as an off-white solid.

LC-MS (ESI): m/z=687.51 [M+H]⁺.

¹H NMR (400 MHZ, DMSO-d₆): δ 10.76 (s, 1H), 8.69-8.66 (s, 1H), 8.03-7.82 (m, 4H), 7.45-7.42 (d, J=12.0 Hz, 1H), 7.17-7.10 (m, 2H), 6.99-6.95 (t, J=15.6 Hz, j=7.4 Hz, 1H), 6.41-6.39 (d, J=7.2 Hz, 1H), 6.36 (s, 1H), 6.31-6.29 (d, J=7.6 Hz, 1H), 5.35 (s, 1H), 4.56 (s, 2H), 3.67 (s, 4H), 3.06-3.05 (m, 3H), 2.66-2.65 (d, J=4.4 Hz, 4H), 2.42-2.40 (m, 1H), 2.05-1.94 (m, 4H), 1.74 (m, 2H), 1.52 (m, 1H), 1.01 (m, 4H).

### Example 13. Synthesis of 2-((6-((5-chloro-2-((((1r,4r)-4-((3-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (Compound 262b)

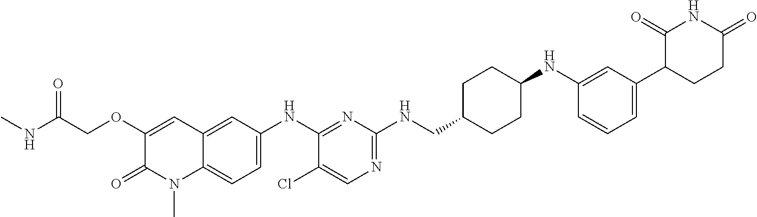 [SMILES: CNC(=O)COc1cc2cc(Nc3nc(NC[C@H]4CC[C@H](Nc5cccc(C6CCC(=O)NC6=O)c5)CC4)ncc3Cl)ccc2n(C)c1=O]

### Preparation of tert-butyl (((1r,4r)-4-((3-(2,6-bis(benzyloxy)pyridin-3-yl)phenyl)amino)cyclohexyl)methyl) carbamate

To a stirred solution of 2,6-bis(benzyloxy)-3-(3-bromophenyl)pyridine (0.75 g, 1.68 mmol) and tert-butyl (((1r,4r)-4-aminocyclohexyl)methyl) carbamate (0.42 g, 1.84 mmol) in 1,4-dioxane (15 mL) was added potassium tert-butoxide (0.56 g, 5.04 mmol). The resulting mixture was degassed with N₂ for 5 minutes, then BrettPhos-Pd (G3) (0.08 g, 0.08 mmol) was added, and the reaction mixture was heated to 80° C. for 3 hours. The reaction was quenched with water and extracted into EtOAc. The combined organic layers were dried over anhydrous sodium sulfate, filtered, and concentrated to get crude product. The crude product was purified by flash column chromatography (SiO₂, 100-200, 20% ethyl acetate in petroleum ether) to afford tert-butyl (((1r,4r)-4-((3-(2,6-bis(benzyloxy)pyridin-3-yl)phenyl)amino)cyclohexyl)methyl) carbamate (0.52 g) as a yellow, sticky liquid.

LC-MS (ESI): m/z=594.82 [M+H]⁺.

### Preparation of tert-butyl (((1r,4r)-4-((3-(2,6-dioxopiperidin-3-yl)phenyl)amino)cyclohexyl)methyl) carbamate

A stirred solution of tert-butyl (((1r,4r)-4-((3-(2,6-bis(benzyloxy)pyridin-3-yl)phenyl)amino)cyclohexyl)methyl) carbamate (0.52 g, 0.88 mmol) in N,N-dimethylformamide (0.5 mL), acetic acid (1.0 mL) and tetrahydrofuran (10 mL) was put under N₂ atmosphere. Palladium hydroxide on carbon, 20% Pd/C (0.52 g, w/w) was added, and the reaction mixture was stirred under hydrogen atmosphere with balloon pressure at room temperature for 16 hours. The reaction mixture was filtered through a celite bed and washed with DCM. The combined organic layers were concentrated to isolate crude product. The crude product was purified by flash column chromatography (SiO₂, 100-200, 50% ethyl acetate in petroleum ether) to afford tert-butyl (((1r,4r)-4-((3-(2,6-dioxopiperidin-3-yl)phenyl)amino)cyclohexyl)methyl) carbamate as a colorless liquid (0.25 g).

LC-MS (ESI): m/z=416.49 [M+H]⁺.

### Preparation of 3-(3-(((1r,4r)-4-(aminomethyl)cyclohexyl)amino)phenyl)piperidine-2,6-dione hydrochloride

To a stirred solution of tert-butyl (((1r,4r)-4-((3-(2,6-dioxopiperidin-3-yl)phenyl)amino)cyclohexyl)methyl) carbamate (0.25 g, 0.60 mmol) in DCM (2.5 mL) was added 4 M HCl in 1,4-dioxane (0.75 mL) at 0° C. under N₂ atmosphere. The resultant reaction mixture was allowed to warm to room temperature over 5 hours with stirring. The reaction mixture was concentrated under reduced pressure to obtain crude product. The crude product was triturated with diethyl ether and n-pentane to afford 3-(3-(((1r,4r)-4-(aminomethyl)cyclohexyl)amino)phenyl)piperidine-2,6-dione hydrochloride (0.19 g) as a white solid.

LC-MS (MSI): m/z=316.41 [M+H]⁺.

### Preparation of 2-((6-((5-chloro-2-((((1r,4r)-4-((3-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide

To a stirred solution of 3-(3-(((1r,4r)-4-(aminomethyl)cyclohexyl)amino)phenyl)piperidine-2,6-dione hydrochloride (0.18 g, 0.51 mmol) in DMSO (5.4 mL), DIPEA (0.45 mL, 2.55 mmol) was added. After 10 minutes, 2-((6-((2,5-dichloropyrimidin-4-yl)amino)-1-methyl-2-oxo-1,2-dihydroquinolin-3-yl)oxy)-N-methylacetamide (0.08 g, 0.20 mmol) was added. The resultant reaction mixture was heated at 100° C. for 16 hours. The reaction was quenched with cold water. The resulting precipitate was filtered, washed with cold water, and dried under vacuo to isolate crude product. The crude product was purified by prep-HPLC to afford 2-((6-((5-chloro-2-((((1r,4r)-4-((3-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide as an off-white solid (0.032 g).

LC-MS (ESI): m/z=687.43 [M+H]⁺.

¹H NMR (400 MHz, DMSO-d₆): δ 10.76 (s, 1H), 8.69-8.66 (s, 1H), 8.03-7.82 (m, 4H), 7.45-7.42 (d, J=12.0 Hz, 1H), 7.17-7.10 (m, 2H), 6.99-6.95 (t, J=15.6 Hz, j=7.4 Hz, 1H), 6.41-6.39 (d, J=7.2 Hz, 1H), 6.36 (s, 1H), 6.31-6.29 (d, J=7.6 Hz, 1H), 5.35 (s, 1H), 4.56 (s, 2H), 3.67 (s, 4H), 3.06-3.05 (m, 3H), 2.66-2.65 (d, J=4.4 Hz, 4H), 2.42-2.40 (m, 1H), 2.05-1.94 (m, 4H), 1.74 (m, 2H), 1.52 (m, 1H), 1.01 (m, 4H).

### Example 14. Synthesis of 2-((6-((5-chloro-2-((3R)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide (Compound 266b)

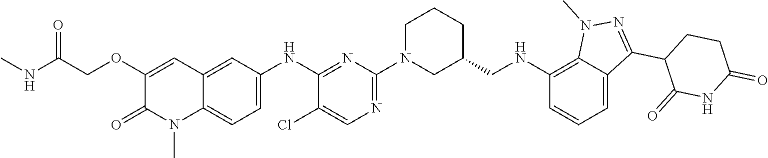 [SMILES: CNC(=O)COc1cc2cc(Nc3nc(N4CCC[C@H](CNc5cccc6c(C7CCC(=O)NC7=O)nn(C)c56)C4)ncc3Cl)ccc2n(C)c1=O]

### Preparation of tert-butyl (R)-3-(((3-(2,6-bis(benzyloxy)pyridin-3-yl)-1-methyl-1H-indazol-7-yl)amino)methyl)piperidine-1-carboxylate

A stirred solution of 3-(2,6-bis(benzyloxy)pyridin-3-yl)-7-bromo-1-methyl-1H-indazole (0.47 g, 0.94 mmol), tert-butyl (R)-3-(aminomethyl)piperidine-1-carboxylate (0.40 g, 1.88 mmol) and potassium tertiary butoxide (0.31 g, 2.40 mmol) in 1,4-dioxane (8.0 mL) was degassed with argon for 10 minutes. Bis(tri-tert-butylphosphine)palladium (0) (0.094 g, 0.48 mmol) was added. After degassing for 5 minutes, the reaction was heated at 80° C. for 2 hours. The reaction mixture was filtered through a celite bed and concentrated under reduced pressure to isolate crude product. The crude product was purified by column chromatography (SiO₂, 100-200 mesh; 30-40% EtOAc in petroleum ether) to afford tert-butyl (R)-3-(((3-(2,6-bis(benzyloxy)pyridin-3-yl)-1-methyl-1H-indazol-7-yl)amino)methyl)piperidine-1-carboxylate (0.40 g) as an off-white solid.

LC-MS (ESI): m/z=634.3 [M+H]⁺.

### Preparation of tert-butyl (3R)-3-(((3-(2,6-dioxopiperidin-3-yl)-1-methyl-1H-indazol-7-yl)amino)methyl)piperidine-1-carboxylate

A solution of tert-butyl (R)-3-(((3-(2,6-bis(benzyloxy)pyridin-3-yl)-1-methyl-1H-indazol-7-yl)amino)methyl)piperidine-1-carboxylate (0.35 g, 0.55 mmol) in THF (19.0 mL), DMF (2.0 mL), 1,4-dioxane (3.5 mL) and acetic acid (0.35 mL) was degassed with nitrogen. Palladium hydroxide on carbon (1.4 g) was added, and the reaction was put under hydrogen atmosphere (15 psi) at room temperature for 4 hours with stirring. The reaction mixture was filtered through a celite bed and concentrated to obtain crude product. The crude product was washed with n-pentane and dried to afford tert-butyl (3R)-3-(((3-(2,6-dioxopiperidin-3-yl)-1-methyl-1H-indazol-7-yl)amino)methyl)piperidine-1-carboxylate (0.31 g) as a brown, gummy solid.

LC-MS (ESI): m/z=456.54 [M+H]⁺.

### Preparation of 3-(1-methyl-7-((((R)-piperidin-3-yl)methyl)amino)-1H-indazol-3-yl)piperidine-2,6-dione

A solution of tert-butyl (3R)-3-(((3-(2,6-dioxopiperidin-3-yl)-1-methyl-1H-indazol-7-yl)amino)methyl)piperidine-1-carboxylate (0.28 g, 0.61 mmol) in DCM (3 mL) was cooled to 0° C. and TFA was added. After allowing to warm to room temperature over 3 hours, the reaction was concentrated under vacuum. Residual solvent was co-distilled with toluene to obtain crude product. The crude product was washed with n-pentane to afford 3-(1-methyl-7-((((R)-piperidin-3-yl)methyl)amino)-1H-indazol-3-yl)piperidine-2,6-dione (0.23 g) as a brown, gummy solid.

LC-MS (ESI): m/z=356.41 [M+H]⁺.

### Preparation of 2-((6-((5-chloro-2-((3R)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-7-((((R)-piperidin-3-yl)methyl)amino)-1H-indazol-3-yl)piperidine-2,6-dione (0.20 g, 0.56 mmol) and 2-((6-((2,5-dichloropyrimidin-4-yl)amino)-1-methyl-2-oxo-1,2-dihydroquinolin-3-yl)oxy)-N-methylacetamide (0.14 g, 0.47 mmol) in DMSO, N,N-diisopropylethylamine (0.58 g, 4.50 mmol) was added. The reaction mixture was stirred at 80° C. for 6 hours. The reaction mixture was poured into ice-cold water. The resulting precipitate was filtered and dried to obtain crude product. The crude product was purified by prep-HPLC to afford 2-((6-((5-chloro-2-((3R)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide (0.062 g) as an off-white solid.

LC-MS (ESI): m/z=727.50 [M+H]⁺.

¹H NMR (400 MHz, DMSO-d₆): δ 10.84 (s, 1H), 8.84 (s, 1H), 8.04 (s, 1H), 7.94-7.92 (m, 2H), 7.78 (d, J=9.20 Hz, 1H), 7.40 (d, J=8.80 Hz, 1H), 7.11 (d, J=4.00 Hz, 1H), 6.94 (dd, J=2.40, 8.20 Hz, 1H), 6.39-6.37 (m, 1H), 6.38 (d, J=6.40 Hz, 1H), 5.32 (br s, 1H), 4.60-4.44 (m, 3H), 4.26-4.12 (m, 5H), 1.99-1.89 (m, 2H), 3.05-2.50 (m, 4H), 2.33-2.50 (m, 5H), 2.18-2.16 (m, 2H), 1.98-1.90 (m, 2H), 1.73-1.69 (m, 2H), 1.37-1.39 (m, 2H).

### Example 15. Synthesis of 2-((6-((5-chloro-2-((3S)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide (Compound 266a)

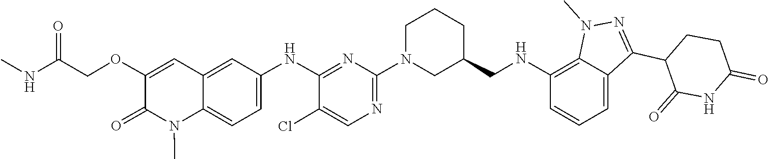 [SMILES: CNC(=O)COc1cc2cc(Nc3nc(N4CCC[C@@H](CNc5cccc6c(C7CCC(=O)NC7=O)nn(C)c56)C4)ncc3Cl)ccc2n(C)c1=O]

### Preparation of tert-butyl(S)-3-(((3-(2,6-bis(benzyloxy)pyridin-3-yl)-1-methyl-1H-indazol-7-yl)amino)methyl)piperidine-1-carboxylate

A solution of 3-(2,6-bis(benzyloxy)pyridin-3-yl)-7-bromo-1-methyl-1H-indazole (0.47 g, 0.94 mmol), tert-butyl(S)-3-(aminomethyl)piperidine-1-carboxylate (0.40 g, 1.88 mmol), and potassium tertiary butoxide (0.31 g, 2.39 mmol) in 1,4-dioxane (8.0 mL) was degassed with argon for 5 minutes. Bis(tri-tert-butylphosphine)palladium (0) (0.094 g, 0.48 mmol) was added, and the reaction was degassed for 5 minutes. The reaction mixture was heated at 80° C. for 2 hours. The reaction mixture was filtered through a celite bed and concentrated to isolate crude product. The crude product was purified by column chromatography (neutral alumina, 1 to 2% methanol in DCM) to afford tert-butyl(S)-3-(((3-(2,6-bis(benzyloxy)pyridin-3-yl)-1-methyl-1H-indazol-7-yl)amino)methyl)piperidine-1-carboxylate (450 mg) as an off-white solid.

LC-MS (ESI): m/z=634.8 [M+H]⁺.

### Preparation of tert-butyl (3S)-3-(((3-(2,6-dioxopiperidin-3-yl)-1-methyl-1H-indazol-7-yl)amino)methyl)piperidine-1-carboxylate

A solution of tert-butyl(S)-3-(((3-(2,6-bis(benzyloxy)pyridin-3-yl)-1-methyl-1H-indazol-7-yl)amino)methyl)piperidine-1-carboxylate (0.40 g, 0.63 mmol) in THF (24 mL), DMF (2.4 mL), 1,4-dioxane (4.0 mL), and acetic acid (0.4 mL) was degassed with nitrogen. Palladium hydroxide on carbon (1.6 g) was added to the reaction. The reaction was put under hydrogen atmosphere (15 psi) and stirred at room temperature for 4 hours. The reaction mixture was filter through a celite bed, and the celite was washed with THF. The filtrate was concentrated to obtain crude product. The crude product was washed with n-pentane and dried to afford tert-butyl (3S)-3-(((3-(2,6-dioxopiperidin-3-yl)-1-methyl-1H-indazol-7-yl)amino)methyl)piperidine-1-carboxylate (0.35 g) as brown, gummy solid.

LC-MS (ESI): m/z=456.54 [M+H]⁺.

### Preparation of 3-(1-methyl-7-((((R)-piperidin-3-yl)methyl)amino)-1H-indazol-3-yl)piperidine-2,6-dione

A stirred solution of tert-butyl (3S)-3-(((3-(2,6-dioxopiperidin-3-yl)-1-methyl-1H-indazol-7-yl)amino)methyl)piperidine-1-carboxylate (0.32 g, 0.70 mmol) in DCM was cooled to 0° C. TFA (3.2 mL) was added to the reaction, and the reaction was allowed to warm to room temperature over 3 hours with stirring. The reaction mixture was concentrated to obtain crude product. The crude product was washed with n-pentane to afford 3-(1-methyl-7-((((R)-piperidin-3-yl)methyl)amino)-1H-indazol-3-yl)piperidine-2,6-dione (0.30 g) as brown, gummy solid.

LC-MS (ESI): m/z=356.18 [M+H]⁺.

### Preparation of 2-((6-((5-chloro-2-((3S)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-7-((((R)-piperidin-3-yl)methyl)amino)-1H-indazol-3-yl)piperidine-2,6-dione (0.27 g, 0.76 mmol) and 2-((6-((2,5-dichloropyrimidin-4-yl)amino)-1-methyl-2-oxo-1,2-dihydroquinolin-3-yl)oxy)-N-methylacetamide (0.12 g, 0.30 mmol) in DMSO was added N,N-diisopropylethylamine (0.78 g, 6.08 mmol). The reaction mixture was stirred at 80° C. for 6 hours. The reaction mixture was poured into ice-cold water and resulted in a precipitate. The precipitate was filtered and dried to obtain crude product. The crude product was purified by prep-HPLC to afford 2-((6-((5-chloro-2-((3S)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide (58 mg) as a brown solid.

LC-MS (ESI): m/z=727.50 [M+H]⁺.

¹H NMR (400 MHZ, DMSO-d₆): δ 10.83 (s, 1H), 8.83 (s, 1H), 8.04 (s, 1H), 7.93-7.89 (m, 2H), 7.79 (d, J=9.20 Hz, 1H), 7.40 (d, J=8.80 Hz, 1H), 7.11 (d, J=4.00 Hz, 1H), 6.94 (d, J=8.20 Hz, 1H), 6.83-6.81 (m, 1H), 6.38 (d, J=6.40 Hz, 1H), 5.38-5.34 (br s, 1H), 4.49 (br s, 3H), 4.27-4.12 (m, 5H), 3.77-3.52 (m, 3H), 3.04-2.91 (m, 4H), 2.65-2.54 (m, 5H), 2.33-2.18 (m, 2H), 1.98-1.90 (m, 2H), 1.73-1.69 (m, 2H), 1.39-1.37 (m, 1H).

### Example 16. Synthesis of 2-((6-((5-chloro-2-(4-(2-((3-(2,6-dioxopiperidin-3-yl)phenyl)amino)ethyl)piperidin-1-yl)pyrimidin-4-yl)amino)-1-methyl-2-oxo-1,2-dihydroquinolin-3-yl)oxy)-N-methylacetamide (Compound 263)

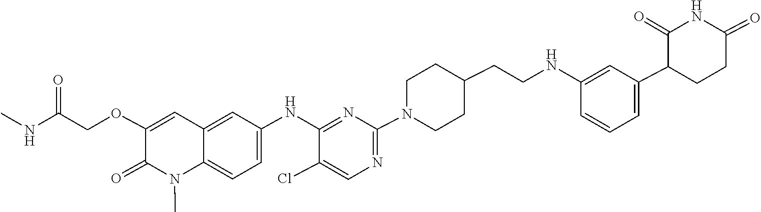 [SMILES: CNC(=O)COc1cc2cc(Nc3nc(N4CCC(CCNc5cccc(C6CCC(=O)NC6=O)c5)CC4)ncc3Cl)ccc2n(C)c1=O]

### Preparation of tert-butyl 4-(2-((3-(2,6-bis(benzyloxy)pyridin-3-yl)phenyl)amino)ethyl)piperidine-1-carboxylate

A stirred solution of tert-butyl 4-(2-aminoethyl)piperidine-1-carboxylate (0.38 g, 1.66 mmol), 2,6-bis(benzyloxy)-3-(3-bromophenyl)pyridine (0.74 g, 1.65 mmol), and potassium tert-butoxide (0.56 g, 5.041 mmol) in 1,4-dioxane (14 mL) was degassed with nitrogen for 15 minutes. BrettPhos Pd G3 (0.966 g, 0.084 mmol) was added, and the resulting mixture was degassed again for 10 minutes. The reaction was heated at 80° C. for 3 hours. The reaction mixture was filtered through a celite bed and concentrated under reduced pressure to isolate crude product. The crude product was purified by flash column chromatography (SiO₂, 100-200, 40% ethyl acetate in petroleum ether) to afford tert-butyl 4-(2-((3-(2,6-bis(benzyloxy)pyridin-3-yl)phenyl)amino)ethyl)piperidine-1-carboxylate (0.550 g) as a brown semi-solid.

LC-MS (ESI): m/z=594.79 [M+H]⁺.

### Preparation of tert-butyl 4-(2-((3-(2,6-dioxopiperidin-3-yl)phenyl)amino)ethyl)piperidine-1-carboxylate

To a stirred solution of tert-butyl 4-(2-((3-(2,6-bis(benzyloxy)pyridin-3-yl)phenyl)amino)ethyl)piperidine-1-carboxylate (0.55 g, 0.927 mmol) in THF (5.5 mL) and DMF (0.55 mL) was added acetic acid (0.27 mL) and palladium hydroxide on carbon, powder, unreduced, 20% Pd, moisture ca 60% (0.51 g, w/w). The reaction was put under hydrogen atmosphere (balloon pressure) and stirred at room temperature for 12 hours. The reaction mixture was filtered through a celite bed, and the filtrate was concentrated under reduced pressure to isolate crude product. The crude product was purified by flash column chromatography (SiO₂, 100-200, 30% ethyl acetate in petroleum ether) to afford tert-butyl 4-(2-((3-(2,6-dioxopiperidin-3-yl)phenyl)amino)ethyl)piperidine-1-carboxylate (0.240 g) as an off-white solid.

LC-MS (ESI): m/z=438 [M+Na]⁺.

### Preparation of 3-(3-((2-(piperidin-4-yl)ethyl)amino)phenyl)piperidine-2,6-dione

To a stirred solution of tert-butyl 4-(2-((3-(2,6-dioxopiperidin-3-yl)phenyl)amino)ethyl)piperidine-1-carboxylate (0.24 g, 0.578 mmol) in DCM (2.4 mL), 4 M HCl in dioxane (1.2 mL) was added dropwise. The reaction was stirred for 16 hours at room temperature. The reaction mixture was concentrated to isolate crude product. The solid was triturated with diethyl ether to afford 3-(3-((2-(piperidin-4-yl)ethyl)amino)phenyl)piperidine-2,6-dione hydrochloride (0.180 g) as a white solid.

LC-MS (ESI): m/z=316.39 [M+H]⁺.

### Preparation of 2-((6-((5-chloro-2-(4-(2-((3-(2,6-dioxopiperidin-3-yl)phenyl)amino)ethyl)piperidin-1-yl)pyrimidin-4-yl)amino)-1-methyl-2-oxo-1,2-dihydroquinolin-3-yl)oxy)-N-methylacetamide

To a stirred solution of 3-(3-((2-(piperidin-4-yl)ethyl)amino)phenyl)piperidine-2,6-dione (0.16 g, 0.508 mmol) in DMSO (4.8 mL), DIPEA (0.88 mL, 5.076 mmol) and p-toluene sulfonic acid (0.004 g, 0.025 mmol) were added. The reaction was degassed with nitrogen for 1 minute and 2-((6-((2,5-dichloropyrimidin-4-yl)amino)-1-methyl-2-oxo-1,2-dihydroquinolin-3-yl)oxy)-N-methylacetamide (0.14 g, 0.35 mmol) was added. The reaction was heated to 165° C. for 3 hours. The reaction mixture was quenched with water (20 mL). The resulting precipitate was filtered and dried under vacuum to isolate crude product. The crude product was purified by prep-HPLC to afford 2-((6-((5-chloro-2-(4-(2-((3-(2,6-dioxopiperidin-3-yl)phenyl)amino)ethyl)piperidin-1-yl)pyrimidin-4-yl)amino)-1-methyl-2-oxo-1,2-dihydroquinolin-3-yl)oxy)-N-methylacetamide (0.032 g) as a white solid.

LC-MS (ESI): m/z=687.43 [M+H]⁺.

¹H NMR (400 MHZ, DMSO-d₆): δ 10.77 (s, 1H), 8.81 (s, 1H), 8.03 (s, 1H), 7.94 (m, J=4.8 Hz, 2H), 7.76 (q, J=3.8 Hz, 1H), 7.47 (d, J=9.1 Hz, 1H), 7.10 (s, 1H), 7.00 (t, J=7.8 Hz, 1H), 6.45 (d, 1H), 6.40 (m, 1H), 6.33 (d, 1H), 5.52 (t, J=5.1 Hz, 1H), 4.57 (s, 2H), 4.51 (bd, 2H) 3.67 (b, 2H), 3.03 (q, J=6.3 Hz, 3H), 2.82 (t, 2H), 2.65 (d, 3H), 2.42 (q, 1H), 2.06 (m, 3H), 1.71 (t, J=16.0 Hz, 3H), 1.50 (t, 2H), 1.09 (q, 2H).

### Example 17. Synthesis of 2-((6-((5-chloro-2-(4-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (Compound 265)

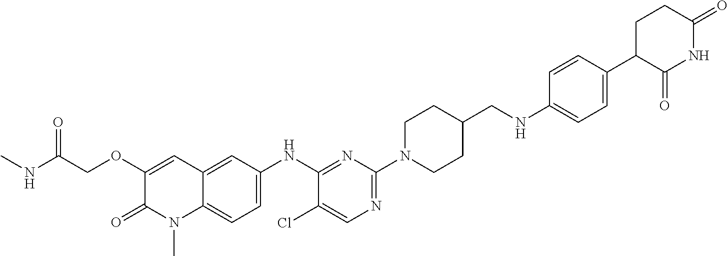 [SMILES: CNC(=O)COc1cc2cc(Nc3nc(N4CCC(CNc5ccc(C6CCC(=O)NC6=O)cc5)CC4)ncc3Cl)ccc2n(C)c1=O]

### Preparation of tert-butyl 4-(((4-(2,6-bis(benzyloxy)pyridin-3-yl)phenyl)amino)methyl)piperidine-1-carboxylate

To a stirred solution of 2,6-bis(benzyloxy)-3-(4-bromophenyl)pyridine (0.75 g, 1.68 mmol) in 1,4-dioxane (15 mL) was added tert-butyl 4-(aminomethyl)piperidine-1-carboxylate (0.43 g, 2.01 mmol) and potassium tert-butoxide (0.56 g, 5.04 mmol). The resulting solution was degassed with nitrogen for 15 minutes, and BrettPhos Pd G3 (0.07 g, 0.08 mmol) was added. The resulting mixture was degassed again for 5 minutes and heated at 80° C. for 3 hours. The reaction mixture was filtered through a celite bed, and the filtrate was concentrated under reduced pressure to isolate crude product. The crude product was purified by flash column chromatography (SiO₂, 100-200, 70% ethyl acetate in petroleum ether) to afford tert-butyl 4-(((4-(2,6-bis(benzyloxy)pyridin-3-yl)phenyl)amino)methyl)piperidine-1-carboxylate (0.70 g) as an off-white solid.

LC-MS (ESI): m/z=580.60 [M+H]⁺.

### Preparation of tert-butyl 4-(((4-(2,6-dioxopiperidin-3-yl)phenyl)amino)methyl)piperidine-1-carboxylate

To a stirred solution of tert-butyl 4-(((4-(2,6-bis(benzyloxy)pyridin-3-yl)phenyl)amino)methyl)piperidine-1-carboxylate (0.7 g, 1.207 mmol) in THF (42 mL), DMF (4.2 mL), 1,4-dioxane (7 mL), acetic acid (0.7 mL), and palladium hydroxide (3.05 g) were added. The reaction mixture was put under hydrogen atmosphere (balloon pressure) and stirred at room temperature for 3 hours. The reaction mixture was filtered through a celite bed, and the filtrate was concentrated under reduced pressure to isolate crude product. The crude product was purified by flash column chromatography (SiO₂, 100-200, 60% ethyl acetate in petroleum ether) to afford tert-butyl 4-(((4-(2,6-dioxopiperidin-3-yl)phenyl)amino)methyl)piperidine-1-carboxylate (0.43 g) as an off-white solid.

LC-MS (ESI): m/z=424.42 [M+H]⁺.

### Preparation of 3-(4-((piperidin-4-ylmethyl)amino)phenyl)piperidine-2,6-dione

To a stirred suspension of tert-butyl 4-(((4-(2,6-dioxopiperidin-3-yl)phenyl)amino)methyl)piperidine-1-carboxylate (0.43 g, 1.07 mmol) in DCM (8.6 mL), 4 M HCl in 1,4-dioxane (4.3 mL) was added at 0° C. The reaction mixture was allowed to warm to room temperature over 3 hours with stirring. The reaction mixture solvent was evaporated under reduced pressure to isolate crude product. The crude product was purified by trituration with n-hexanes to afford 3-(4-((piperidin-4-ylmethyl)amino)phenyl)piperidine-2,6-dione (0.27 g) as an off-white solid.

LC-MS (ESI): m/z=302.13 [M+H]⁺.

### Preparation of 2-((6-((5-chloro-2-(4-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(4-((piperidin-4-ylmethyl)amino)phenyl)piperidine-2,6-dione (0.25 g, 0.82 mmol) and 2-((6-((2,5-dichloropyrimidin-4-yl)amino)-1-methyl-2-oxo-1,2-dihydroquinolin-3-yl)oxy)-N-methylacetamide (0.23 g, 0.58 mmol) in DMSO (5 mL), DIPEA (0.53 mL, 4.14 mmol) was added. The resulting reaction mixture was stirred at 100° C. for 3 hours. Water was added to the reaction, and the resulting precipitate was filtered off to isolate crude product. The crude product was purified by prep HPLC to afford 2-((6-((5-chloro-2-(4-(((4-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (70 mg) as an off-white solid.

LC-MS (ESI): m/z=673.44 [M+H]⁺.

¹H NMR (400 MHZ, DMSO-d₆): δ 10.85 (s, 1H), 8.82 (s, 1H), 8.03 (s, 1H), 7.94 (m, 2H), 7.74 (m, 1H), 7.46 (d, J=12 Hz, 1H), 7.08 (s, 1H), 6.88 (d, J=8 Hz, 2H), 6.52 (d, J=8 Hz, 2H), 5.62 (d, J=4 Hz, 1H), 4.53 (t, 4H), 3.60 (m, 4H), 2.60 (m, 4H), 2.54 (m, 4H), 2.50 (m, 2H), 2.43 (m, 1H), 2.06 (m, 2H), 1.78 (m, J=12 Hz, 3H), 1.11 (m, J=8 Hz, 2H).

### Example 18. Synthesis of 2-((6-((5-chloro-2-((3S)-3-(((3-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (Compound 275b)

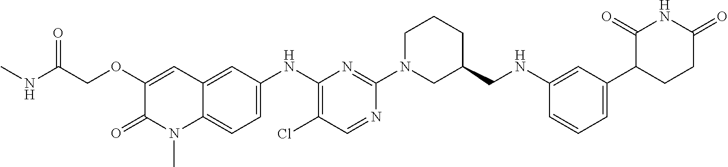 [SMILES: CNC(=O)COc1cc2cc(Nc3nc(N4CCC[C@@H](CNc5cccc(C6CCC(=O)NC6=O)c5)C4)ncc3Cl)ccc2n(C)c1=O]

### Preparation of tert-butyl(S)-3-(((3-(2,6-bis(benzyloxy)pyridin-3-yl)phenyl)amino)methyl)piperidine-1-carboxylate

To a stirred solution of 2,6-bis(benzyloxy)-3-(3-bromophenyl)pyridine (1.50 g, 3.36 mmol) and tert-butyl(S)-3-(aminomethyl)piperidine-1-carboxylate (1.08 g, 5.04 mmol) in 1,4-dioxane (30.0 mL), potassium tert-butoxide (1.11 g, 10.08 mmol) was added. The reaction was degassed with argon for 10 minutes, then bis(tri-tert-butylphosphine)palladium (0) (0.05 g, 0.10 mmol) was added, and the reaction was degassed for an additional 5 minutes. The reaction mixture was stirred at 80° C. for 4 hours. The reaction mixture was cooled to room temperature and filtered through a celite bed. The filtrate was concentrated to isolate crude product. The crude product was purified by flash column chromatography (SiO₂, 230-400, 8% ethyl acetate in petroleum ether) to afford tert-butyl(S)-3-(((3-(2,6-bis(benzyloxy)pyridin-3-yl)phenyl)amino)methyl)piperidine-1-carboxylate (1.15 g, 59%) as a pale yellow, sticky compound.

LC-MS (ESI): m/z=580.72 [M+H]⁺.

### Preparation of tert-butyl (3S)-3-(((3-(2,6-dioxopiperidin-3-yl)phenyl)amino)methyl)piperidine-1-carboxylate

To a stirred solution of tert-butyl(S)-3-(((3-(2,6-bis(benzyloxy)pyridin-3-yl)phenyl)amino)methyl)piperidine-1-carboxylate (1.20 g, 1.89 mmol) in THF (33 mL) was added acetic acid (1.2 mL). The reaction was degassed with nitrogen for 5 minutes, and palladium hydroxide on carbon (2.20 g) was added to the reaction. The reaction mixture was put under hydrogen atmosphere (15 psi) and stirred at room temperature for 4 hours. The reaction mixture was filtered through a celite bed and washed with THF:DCM (1:1). The filtrate was concentrated and dried to isolate crude product. The crude product was washed with n-pentane and dried to afford tert-butyl (3S)-3-(((3-(2,6-dioxopiperidin-3-yl)phenyl)amino)methyl)piperidine-1-carboxylate (0.85 g) as a brown, sticky compound.

LC-MS (ESI): m/z=402.42 [M+H]⁺.

### Preparation of 3-(3-((((R)-piperidin-3-yl)methyl)amino)phenyl)piperidine-2,6-dione

To a stirred solution of tert-butyl (3S)-3-(((3-(2,6-dioxopiperidin-3-yl)phenyl)amino)methyl)piperidine-1-carboxylate (0.85 g, 2.12 mmol) in DCM (4.0 mL) cooled to 0° C., TFA (5.1 mL) was added. The reaction mixture was allowed to warm to room temperature over 3 hours with stirring. The reaction mixture was concentrated to isolate crude product. The crude product was triturated with petroleum ether to afford 3-(3-((((R)-piperidin-3-yl)methyl)amino)phenyl)piperidine-2,6-dione (0.80 g) as a brown liquid.

LC-MS (ESI): m/z=302.19 [M+H]⁺.

### Preparation of 2-((6-((5-chloro-2-((3S)-3-(((3-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(3-((((R)-piperidin-3-yl)methyl)amino)phenyl)piperidine-2,6-dione (0.35 g, 1.16 mmol) and 2-((6-((2,5-dichloropyrimidin-4-yl)amino)-1-methyl-2-oxo-1,2-dihydroquinolin-3-yl)oxy)-N-methylacetamide (0.19 g, 0.46 mmol) in DMSO, N,N-diisopropylethylamine (1.62 mL, 9.29 mmol) was added. The reaction mixture was stirred at 100° C. for 5 hours. The reaction mixture was cooled to room temperature and poured into ice water. The resulting precipitate was filtered and dried to isolate crude product. The crude product was purified by preparative HPLC purification to afford 2-((6-((5-chloro-2-((3S)-3-(((3-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (71 mg) as a pale brown solid.

LC-MS (ESI): m/z=673.43 [M+H]⁺.

¹H NMR (400 MHZ, DMSO-d₆): δ 10.79 (s, 1H), 8.82 (s, 1H), 8.03 (d, J=0.80 Hz, 1H), 7.93-7.80 (m, 3H), 7.43 (d, J=9.20 Hz, 1H), 7.13 (s, 1H), 6.96 (t, J=7.20 Hz, 1H), 6.39-6.32 (m, 3H), 5.62 (br s, 1H), 4.55-4.43 (m, 3H), 4.30 (d, J=13.20 Hz, 1H), 3.63 (s, 4H), 2.94-2.88 (m, 3H), 2.77-2.75 (m, 1H), 2.66 (d, J=4.00 Hz, 3H), 2.59-2.40 (m, 2H), 2.07-2.00 (m, 2H), 1.89 (d, J=9.60 Hz, 1H), 1.75-1.67 (m, 2H), 1.27-1.23 (m, 2H).

### Example 19. Synthesis of 2-((6-((5-chloro-2-((2S)-2-(((3-(2,6-dioxopiperidin-3-yl)-1-methyl-1H-indazol-6-yl)amino)methyl)pyrrolidin-1-yl)pyrimidin-4-yl)amino)-1-methyl-2-oxo-1,2-dihydroquinolin-3-yl)oxy)-N-methylacetamide (Compound 279a)

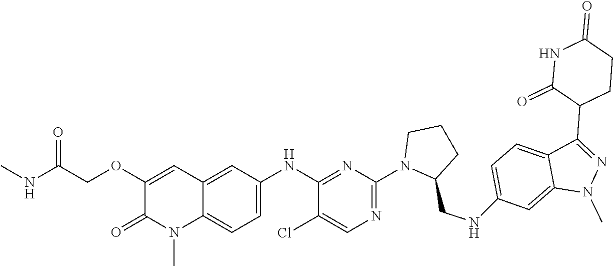 [SMILES: CNC(=O)COc1cc2cc(Nc3nc(N4CCC[C@H]4CNc4ccc5c(C6CCC(=O)NC6=O)nn(C)c5c4)ncc3Cl)ccc2n(C)c1=O]

### Preparation of tert-butyl(S)-2-(((3-(2,6-bis(benzyloxy)pyridin-3-yl)-1-methyl-1H-indazol-6-yl)amino)methyl)pyrrolidine-1-carboxylate

A solution of 3-(2,6-bis(benzyloxy)pyridin-3-yl)-6-bromo-1-methyl-1H-indazole (0.70 g, 1.4 mmol), tert-butyl(S)-2-(aminomethyl)pyrrolidine-1-carboxylate (0.33 g, 1.68 mmol), and sodium tertiary butoxide (0.40 g, 4.2 mmol) in 1,4-dioxane (14 mL) was degassed with argon for 5 minutes. BrettPhos Pd G3 (0.09 g, 0.1 mmol) was added, and the reaction was degassed for 5 minutes. The reaction mixture was heated at 100° C. for 4 hours. The reaction mixture was filtered through a pad of celite and concentrated to obtain crude product. The crude product was purified by column chromatography (neutral alumina, product eluted at 2% methanol in dichloromethane) to afford tert-butyl(S)-2-(((3-(2,6-bis(benzyloxy)pyridin-3-yl)-1-methyl-1H-indazol-6-yl)amino)methyl)pyrrolidine-1-carboxylate (0.70 g) as a brown liquid.

LC-MS (ESI): m/z=620.58 [M+H]⁺.

### Preparation of tert-butyl (2S)-2-(((3-(2,6-dioxopiperidin-3-yl)-1-methyl-1H-indazol-6-yl)amino)methyl)pyrrolidine-1-carboxylate

To a stirred solution of tert-butyl(S)-2-(((3-(2,6-bis(benzyloxy)pyridin-3-yl)-1-methyl-1H-indazol-6-yl)amino)methyl)pyrrolidine-1-carboxylate (0.70 g, 1.13 mmol) in THF (42 mL), 20% Pd(OH)₂ on carbon (2.1 g) was added. The reaction was put under hydrogen atmosphere (15 psi) and stirred at room temperature for 2 hours. The reaction mixture was filtered through a pad of celite and concentrated to obtain crude product. The crude product was washed with n-pentane and dried to afford tert-butyl (2S)-2-(((3-(2,6-dioxopiperidin-3-yl)-1-methyl-1H-indazol-6-yl)amino)methyl)pyrrolidine-1-carboxylate (0.42 g) as a brown solid.

LC-MS (ESI): m/z=442.01 [M+H]⁺.

### Preparation of 3-(1-methyl-6-((((S)-pyrrolidin-2-yl)methyl)amino)-1H-indazol-3-yl)piperidine-2,6-dione

A stirred solution of tert-butyl (2S)-2-(((3-(2,6-dioxopiperidin-3-yl)-1-methyl-1H-indazol-6-yl)amino)methyl)pyrrolidine-1-carboxylate (0.40 g, 0.90 mmol) in DCM (16 mL) was cooled to 0° C. TFA (4 mL) was added, and the reaction mixture was allowed to warm to room temperature over 4 hours with stirring. The reaction mixture was concentrated to obtain crude product. The crude product was washed with n-pentane to afford 3-(1-methyl-6-((((S)-pyrrolidin-2-yl)methyl)amino)-1H-indazol-3-yl)piperidine-2,6-dione (0.25 g) as a brown gummy solid.

LC-MS (ESI): m/z=342.40 [M+H]⁺.

### Preparation of 2-((6-((5-chloro-2-((2S)-2-(((3-(2,6-dioxopiperidin-3-yl)-1-methyl-1H-indazol-6-yl)amino)methyl)pyrrolidin-1-yl)pyrimidin-4-yl)amino)-1-methyl-2-oxo-1,2-dihydroquinolin-3-yl)oxy)-N-methylacetamide

To a stirred solution 3-(1-methyl-6-((((S)-pyrrolidin-2-yl)methyl)amino)-1H-indazol-3-yl)piperidine-2,6-dione (0.22 g, 0.64 mmol) and 2-((6-((2,5-dichloropyrimidin-4-yl)amino)-1-methyl-2-oxo-1,2-dihydroquinolin-3-yl)oxy)-N-methylacetamide (0.10 g, 0.25 mmol) in DMSO, N,N-diisopropylethylamine (0.66 g, 4.4 mmol) was added. The reaction mixture was stirred at 100° C. for 3 hours. The reaction mixture was poured into ice-cold water and stirred for 15 minutes. The resulting precipitate was filtered and dried to afford crude product. The crude product was purified by prep-HPLC to afford 2-((6-((5-chloro-2-((2S)-2-(((3-(2,6-dioxopiperidin-3-yl)-1-methyl-1H-indazol-6-yl)amino)methyl)pyrrolidin-1-yl)pyrimidin-4-yl)amino)-1-methyl-2-oxo-1,2-dihydroquinolin-3-yl)oxy)-N-methylacetamide (17 mg) as an off-white solid.

LC-MS (ESI): m/z=713.43 [M+H]⁺.

¹H NMR (400 MHz, DMSO-d₆): δ 10.90 (s, 1H), 8.82 (s, 1H), 8.22-7.71 (m, 3H), 7.48-7.02 (m, 3H), 6.77-5.84 (m, 2H), 4.57-4.45 (m, 2H), 4.35-4.18 (m, 2H), 3.83 (s, 3H), 3.68-3.33 (m, 8H), 3.10-2.95 (m, 2H), 2.67-2.53 (m, 5H), 2.17-2.14 (m, 2H), 2.03-2.01 (m, 2H), 1.27-1.22 (m, 1H).

### Example 20. Synthesis of 2-((6-((5-chloro-2-((3S)-3-((3-(2,6-dioxopiperidin-3-yl)-1-methyl-1H-indazol-6-yl)amino)piperidin-1-yl)pyrimidin-4-yl)amino)-1-methyl-2-oxo-1,2-dihydroquinolin-3-yl)oxy)-N-methylacetamide (Compound 280a)

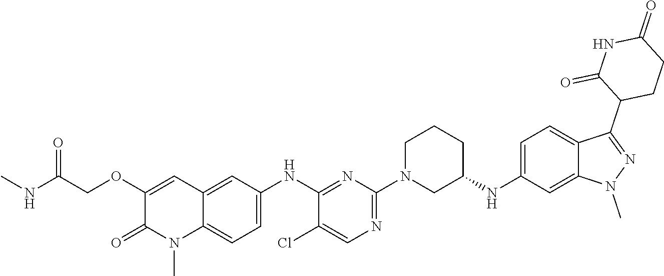 [SMILES: CNC(=O)COc1cc2cc(Nc3nc(N4CCC[C@H](Nc5ccc6c(C7CCC(=O)NC7=O)nn(C)c6c5)C4)ncc3Cl)ccc2n(C)c1=O]

### Preparation of tert-butyl(S)-3-((3-(2,6-bis(benzyloxy)pyridin-3-yl)-1-methyl-1H-indazol-6-yl)amino)piperidine-1-carboxylate

A solution of 3-(2,6-bis(benzyloxy)pyridin-3-yl)-6-bromo-1-methyl-1H-indazole (0.7 g, 1.4 mmol), tert-butyl-(S)-3-aminopiperidine-1-carboxylate (0.33 g, 1.68 mmol) and sodium tertiary butoxide (0.40 g, 4.2 mmol) in 1,4-dioxane (14 mL) was degassed with argon for 5 minutes. BrettPhos Pd G3 (0.12 g, 0.14 mmol) was added, and the reaction was degassed for 5 minutes. The reaction mixture was heated at 100° C. for 4 hours. The reaction mixture was filtered through a pad of celite and concentrated to obtain crude product. The crude product was purified by column chromatography (neutral alumina, product eluted at 2% methanol in dichloromethane) to afford tert-butyl(S)-3-((3-(2,6-bis(benzyloxy)pyridin-3-yl)-1-methyl-1H-indazol-6-yl)amino)piperidine-1-carboxylate (0.70 g) as a brown liquid.

LC-MS (ESI): m/z=620.36 [M+H]⁺.

### Preparation of tert-butyl (3S)-3-((3-(2,6-dioxopiperidin-3-yl)-1-methyl-1H-indazol-6-yl)amino)piperidine-1-carboxylate

To a stirred solution of tert-butyl(S)-3-((3-(2,6-bis(benzyloxy)pyridin-3-yl)-1-methyl-1H-indazol-6-yl)amino)piperidine-1-carboxylate (0.70 g, 1.13 mmol) in THF (42 mL), 20% Pd(OH)₂ on carbon (1.4 g) was added. The reaction was put under hydrogen atmosphere (balloon pressure) and stirred at room temperature for 4 hours. The reaction mixture was filtered through a pad of celite and concentrated to obtain crude product. The crude product was washed with n-pentane and dried to afford tert-butyl (3S)-3-((3-(2,6-dioxopiperidin-3-yl)-1-methyl-1H-indazol-6-yl)amino)piperidine-1-carboxylate (0.45 g) as a brown solid.

LC-MS (ESI): m/z=442.52 [M+H]⁺.

### Preparation of 3-(1-methyl-6-(((S)-piperidin-3-yl)amino)-1H-indazol-3-yl)piperidine-2,6-dione

A stirred solution of tert-butyl (3S)-3-((3-(2,6-dioxopiperidin-3-yl)-1-methyl-1H-indazol-6-yl)amino)piperidine-1-carboxylate (0.45 g, 1.01 mmol) in DCM (9 mL) was cooled to 0° C. TFA (4.5 mL) was added, and the reaction mixture was allowed to warm to room temperature over 4 hours with stirring. The reaction mixture was concentrated to obtain crude product. The crude product was washed with n-pentane to afford 3-(1-methyl-6-(((S)-piperidin-3-yl)amino)-1H-indazol-3-yl)piperidine-2,6-dione (0.30 g) as a brown gummy solid.

LC-MS (ESI): m/z=342.44 [M+H]⁺.

### Preparation of 2-((6-((5-chloro-2-((3S)-3-((3-(2,6-dioxopiperidin-3-yl)-1-methyl-1H-indazol-6-yl)amino)piperidin-1-yl)pyrimidin-4-yl)amino)-1-methyl-2-oxo-1,2-dihydroquinolin-3-yl)oxy)-N-methylacetamide

To a stirred solution 3-(1-methyl-6-(((S)-piperidin-3-yl)amino)-1H-indazol-3-yl)piperidine-2,6-dione (0.30 g, 0.87 mmol) and 2-((6-((2,5-dichloropyrimidin-4-yl)amino)-1-methyl-2-oxo-1,2-dihydroquinolin-3-yl)oxy)-N-methylacetamide (0.17 g, 0.43 mmol) in DMSO, N,N-diisopropylethylamine (0.90 g, 7.0 mmol) was added. The reaction mixture was stirred at 100° C. for 6 hours. The reaction mixture was poured into ice-cold water and stirred for 15 minutes. The resulting precipitate was filtered and dried to obtain crude product. The crude product was purified by prep-HPLC to afford 2-((6-((5-chloro-2-((3S)-3-((3-(2,6-dioxopiperidin-3-yl)-1-methyl-1H-indazol-6-yl)amino)piperidin-1-yl)pyrimidin-4-yl)amino)-1-methyl-2-oxo-1,2-dihydroquinolin-3-yl)oxy)-N-methylacetamide (0.37 mg) as an off-white solid.

LC-MS (ESI): m/z=713.50 [M+H]⁺.

¹H NMR (400 MHZ, DMSO-d₆): δ 10.77 (d, J=12.0 Hz, 1H), 8.83 (s, 1H), 8.07 (s, 1H), 7.93-7.77 (m, 3H), 7.33-7.29 (m, 1H), 6.96-6.84 (br, 2H), 6.56-6.47 (m, 2H), 5.94-5.90 (m, 1H), 4.83-4.75 (m, 1H), 4.43 (br s, 3H), 4.15 (s, 1H), 3.50-3.36 (br, 6H), 3.09-2.92 (m, 1H), 2.72-2.51 (m, 7H), 2.29-2.18 (m, 2H), 2.07-2.06 (m, 1H), 1.77 (br s, 1H), 1.56-1.54 (m, 2H).

### Example 21. Synthesis of 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydroquinolin-3-yl)oxy)-N-methylacetamide (Compound 281)

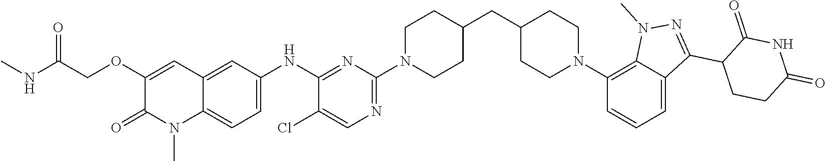 [SMILES: CNC(=O)COc1cc2cc(Nc3nc(N4CCC(CC5CCN(c6cccc7c(C8CCC(=O)NC8=O)nn(C)c67)CC5)CC4)ncc3Cl)ccc2n(C)c1=O]

### Preparation of tert-butyl 4-((1-(3-(2,6-bis(benzyloxy)pyridin-3-yl)-1-methyl-1H-indazol-7-yl)piperidin-4-yl)methyl)piperidine-1-carboxylate

A solution of tert-butyl 4-(piperidin-4-ylmethyl)piperidine-1-carboxylate (0.47 g, 1.66 mmol), 3-(2,6-bis(benzyloxy)pyridin-3-yl)-7-bromo-1-methyl-1H-indazole (0.65 g, 1.33 mmol), and sodium tert-butoxide (0.39 g, 4.16 mmol) in 1,4-dioxane (10 mL) was degassed with nitrogen for 15 minutes. Pd-PEPPSI-iHEPT-Cl (0.08 g, 0.083 mmol) was added, and the reaction was degassed for 10 minutes. The reaction was heated at 80° C. for 6 hours. The reaction was filtered through a pad of celite, and the filtrate was concentrated under reduced pressure to obtain crude product. The crude product was purified by flash column chromatography (SiO₂, 100-200, 12% ethyl acetate in petroleum ether) to afford tert-butyl 4-((1-(3-(2,6-bis(benzyloxy)pyridin-3-yl)-1-methyl-1H-indazol-7-yl)piperidin-4-yl)methyl)piperidine-1-carboxylate (0.37 g) as a brown semi-solid

LC-MS (ESI): m/z=702.88 [M+H]⁺.

### Preparation of tert-butyl 4-((1-(3-(2,6-dioxopiperidin-3-yl)-1-methyl-1H-indazol-7-yl)piperidin-4-yl)methyl)piperidine-1-carboxylate

To stirred solution of tert-butyl 4-((1-(3-(2,6-bis(benzyloxy)pyridin-3-yl)-1-methyl-1H-indazol-7-yl)piperidin-4-yl)methyl)piperidine-1-carboxylate (0.37 g, 0.527 mmol) in ethyl acetate (11.1 mL), palladium on activated carbon (0.37 g) was added. The reaction mixture was put under hydrogen (balloon pressure) and stirred at room temperature for 12 hours. The reaction mixture was filtered through a pad of celite, and the filtrate was concentrated under reduced pressure to obtain crude product. The crude was purified by flash column chromatography (SiO₂, 100-200, 26% ethyl acetate in petroleum ether) to afford tert-butyl 4-((1-(3-(2,6-dioxopiperidin-3-yl)-1-methyl-1H-indazol-7-yl)piperidin-4-yl)methyl)piperidine-1-carboxylate (0.27 g) as an off-white solid.

LC-MS (ESI): m/z=524.62 [M+H]⁺.

### Preparation of 3-(1-methyl-7-(4-(piperidin-4-ylmethyl)piperidin-1-yl)-1H-indazol-3-yl)piperidine-2,6-dione (hydrochloride salt

To a stirred solution of tert-butyl 4-((1-(3-(2,6-dioxopiperidin-3-yl)-1-methyl-1H-indazol-7-yl)piperidin-4-yl)methyl)piperidine-1-carboxylate (0.27 g, 0.516 mmol) in DCM (5 mL), 4 M HCl in dioxane (2.2 mL) was added dropwise. The reaction was stirred for 16 hours at room temperature. The reaction was concentrated via rotary evaporator to obtain crude product. The crude product was triturated with diethyl ether to afford 3-(1-methyl-7-(4-(piperidin-4-ylmethyl)piperidin-1-yl)-1H-indazol-3-yl)piperidine-2,6-dione hydrochloride salt (0.27 g) as a white solid.

LC-MS (ESI): m/z=424.54 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydroquinolin-3-yl)oxy)-N-methylacetamide

A stirred solution of 3-(1-methyl-7-(4-(piperidin-4-ylmethyl)piperidin-1-yl)-1H-indazol-3-yl)piperidine-2,6-dione hydrochloride (0.27 g, 0.587 mmol), DIPEA (1.02 mL, 5.87 mmol), and p-toluenesulfonic acid (0.0052 g, 0.029 mmol) in DMSO (8.1 mL) was degassed with nitrogen for 1 minute. 2-((6-((2,5-dichloropyrimidin-4-yl)amino)-1-methyl-2-oxo-1,2-dihydroquinolin-3-yl)oxy)-N-methylacetamide (0.094 g, 0.307 mmol) was added, and the reaction was heated at 165° C. for 3 hours. The reaction mixture was quenched with water (20 mL). The precipitated solid was filtered and dried under vacuum to obtain crude product. The crude product was purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydroquinolin-3-yl)oxy)-N-methylacetamide (0.092 g) as a white solid.

LC-MS (ESI): m/z=793.50 [M−H]⁻.

¹H NMR (400 MHZ, DMSO-d₆): δ 10.87 (s, 1H), 8.82 (s, 1H), 8.04 (s, 1H), 7.95 (t, J=4.7 Hz, 2H), 7.75 (q, J=3.8 Hz, 1H), 7.48 (d, J=9.2 Hz, 1H), 7.36 (q, J=3.0 Hz, 1H), 7.10 (s, 1H), 7.00 (t, J=3.1 Hz, 2H), 4.58 (s, 2H), 4.50 (d, J=12.4 Hz, 2H), 4.33 (q, J=4.9 Hz, 1H), 4.23 (s, 3H), 3.68 (s, 3H), 3.21 (m, 2H), 2.85 (d, J=12.6 Hz, 2H), 2.65 (q, J=6.3 Hz, 7H), 2.32 (m, J=6.2 Hz, 1H), 2.16 (q, J=6.3 Hz, 1H), 1.70 (m, 6H), 1.37 (m, 2H), 1.23 (s, 4H), 1.05 (d, 2H).

### Example 22. Synthesis of 2-((6-((5-chloro-2-(4-(1-(3-(2,6-dioxopiperidin-3-yl)-1-methyl-1H-indazol-6-yl) azetidin-3-yl)piperazin-1-yl)pyrimidin-4-yl)amino)-1-methyl-2-oxo-1,2-dihydroquinolin-3-yl)oxy)-N-methylacetamide (Compound 282)

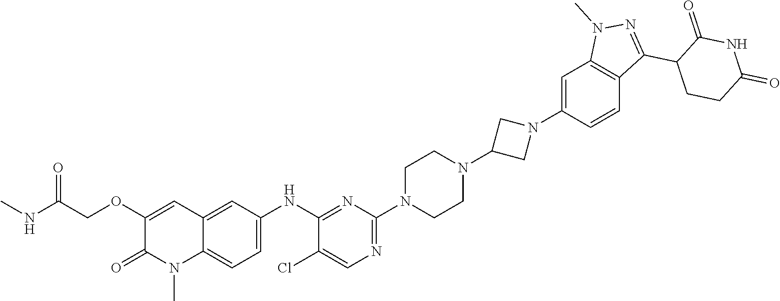 [SMILES: CNC(=O)COc1cc2cc(Nc3nc(N4CCN(C5CN(c6ccc7c(C8CCC(=O)NC8=O)nn(C)c7c6)C5)CC4)ncc3Cl)ccc2n(C)c1=O]

### Preparation of tert-butyl 4-(1-(3-(2,6-bis(benzyloxy)pyridin-3-yl)-1-methyl-1H-indazol-6-yl) azetidin-3-yl)piperazine-1-carboxylate

To a stirred solution of 3-(2,6-bis(benzyloxy)pyridin-3-yl)-6-bromo-1-methyl-1H-indazole (0.6 g, 1.19 mmol) in 1,4-dioxane (12 mL), tert-butyl 4-(azetidin-3-yl)piperazine-1-carboxylate (0.26 g, 1.31 mmol) was added. The reaction was purged with argon for 15 minutes. Cesium carbonate (1.26 g, 3.59 mmol), RuPhos (0.05 g, 0.11 mmol), and RuPhos Pd G3 (0.05 g, 0.06 mmol) were added, and the reaction was heated to 100° C. for 16 hours. The reaction mixture was quenched with water and extracted into ethyl acetate. The combined organic layers were washed with brine solution, dried over anhydrous Na₂SO₄, filtered, and concentrated under vacuum to obtain crude product. The crude product was purified by flash column chromatography (SiO₂, 230-400, 90% ethyl acetate in petroleum ether) to afford tert-butyl 4-(1-(3-(2,6-bis(benzyloxy)pyridin-3-yl)-1-methyl-1H-indazol-6-yl) azetidin-3-yl)piperazine-1-carboxylate (0.64 g) as a light yellow gummy solid.

LC-MS (ESI): m/z=661.69 [M+H]⁺.

### Preparation of tert-butyl 4-(1-(3-(2,6-dioxopiperidin-3-yl)-1-methyl-1H-indazol-6-yl) azetidin-3-yl)piperazine-1-carboxylate

To a stirred solution of tert-butyl 4-(1-(3-(2,6-bis(benzyloxy)pyridin-3-yl)-1-methyl-1H-indazol-6-yl) azetidin-3-yl)piperazine-1-carboxylate (0.54 g, 0.81 mmol) and acetic acid (0.6 mL) in THF (10 mL)/DMF (0.6 mL), 20% Pd(OH)₂ on carbon (0.68 g, 4.90 mmol) was added. The reaction was put under hydrogen atmosphere (15 psi) and stirred at room temperature for 16 hours. The reaction mixture was diluted with DCM and filtered through a pad of celite. The celite was washed with 30% THF in DCM. The filtrate was concentrated under vacuum to get afford tert-butyl 4-(1-(3-(2,6-dioxopiperidin-3-yl)-1-methyl-1H-indazol-6-yl) azetidin-3-yl)piperazine-1-carboxylate (0.50 g) as a light yellow gummy solid.

LC-MS (ESI): m/z=483.55 [M+H]⁺.

### Preparation of 3-(1-methyl-6-(3-(piperazin-1-yl) azetidin-1-yl)-1H-indazol-3-yl)piperidine-2,6-dione

To a stirred solution of tert-butyl 4-(1-(3-(2,6-dioxopiperidin-3-yl)-1-methyl-1H-indazol-6-yl) azetidin-3-yl)piperazine-1-carboxylate (0.35 g, 0.72 mmol) in DCM (3.5 mL), 4 M HCl in dioxane (3.5 mL) was added. The reaction was stirred at room temperature for 2 hours. The reaction mixture was concentrated under vacuum to afford 3-(1-methyl-6-(3-(piperazin-1-yl) azetidin-1-yl)-1H-indazol-3-yl)piperidine-2,6-dione (0.35 g) as an off-white gummy solid.

LC-MS (ESI): m/z=383.47 [M+H]⁺.

### Preparation of 2-((6-((5-chloro-2-(4-(1-(3-(2,6-dioxopiperidin-3-yl)-1-methyl-1H-indazol-6-yl) azetidin-3-yl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-6-(3-(piperazin-1-yl) azetidin-1-yl)-1H-indazol-3-yl)piperidine-2,6-dione (0.35 g, 0.91 mmol) in DMSO (4.2 mL), 2-((6-((2,5-dichloropyrimidin-4-yl)amino)-1-methyl-2-oxo-1,2-dihydroquinolin-3-yl)oxy)-N-ethylacetamide (0.11 g, 0.27 mmol) and DIPEA (1.26 mL, 7.28 mmol) were added. The reaction was heated at 100° C. for 6 hours. The reaction mixture was quenched with ice-cold water. The resulting solid precipitate was filtered and dried under vacuum to obtain crude product. The crude product was purified by preparative HPLC to afford 2-((6-((5-chloro-2-(4-(1-(3-(2,6-dioxopiperidin-3-yl)-1-methyl-1H-indazol-6-yl) azetidin-3-yl)piperazin-1-yl)pyrimidin-4-yl)amino)-1-methyl-2-oxo-1,2-dihydroquinolin-3-yl)oxy)-N-methylacetamide (0.0223 g) as a pale brown solid.

LC-MS (ESI): m/z=754.52 [M+H]⁺.

¹H NMR (400 MHZ, DMSO-d₆): δ 10.90 (s, 1H), 8.89 (s, 1H), 8.07 (s, 1H), 7.97-7.92 (m, 2H), 7.75-7.73 (m, 1H), 7.50-7.46 (m, 2H), 7.14 (s, 1H), 6.39-6.35 (m, 2H), 4.59 (s, 2H), 4.25-4.22 (m, 1H), 4.03-3.99 (m, 2H), 3.86 (s, 3H), 3.70-3.66 (m, 9H), 3.21-3.20 (m, 1H), 2.67-2.51 (m, 5H), 2.41 (s, 4H), 2.32-2.11 (m, 2H).

### Example 23. Synthesis of 2-((6-((5-chloro-2-(4-(1-(3-(2,6-dioxopiperidin-3-yl)-1-methyl-1H-indazol-7-yl) azetidin-3-yl)piperazin-1-yl)pyrimidin-4-yl)amino)-1-methyl-2-oxo-1,2-dihydroquinolin-3-yl)oxy)-N-methylacetamide (Compound 283)

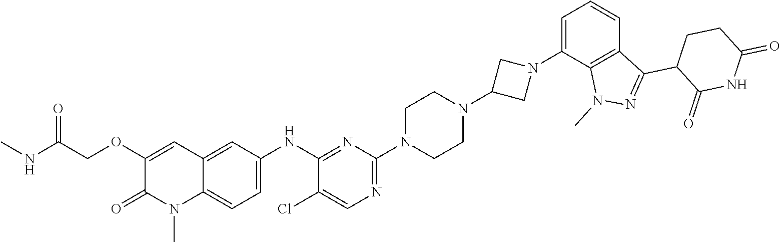 [SMILES: CNC(=O)COc1cc2cc(Nc3nc(N4CCN(C5CN(c6cccc7c(C8CCC(=O)NC8=O)nn(C)c67)C5)CC4)ncc3Cl)ccc2n(C)c1=O]

### Preparation of tert-butyl 4-(1-(3-(2,6-bis(benzyloxy)pyridin-3-yl)-1-methyl-1H-indazol-7-yl) azetidin-3-yl)piperazine-1-carboxylate

To a stirred solution of 3-(2,6-bis(benzyloxy)pyridin-3-yl)-7-bromo-1-methyl-1H-indazole (0.6 g, 1.19 mmol) in 1,4-dioxane (12 mL), tert-butyl 4-(azetidin-3-yl)piperazine-1-carboxylate (0.26 g, 1.31 mmol) was added. The reaction was purged with argon for 15 minutes. Cesium carbonate (1.17 g, 3.59 mmol), RuPhos (0.05 g, 0.11 mmol), and RuPhos Pd G3 (0.05 g, 0.06 mmol) were added, and the reaction was heated at 80° C. for 16 hours. The reaction mixture was quenched with water and extracted into ethyl acetate. The combined organic layers were washed with brine solution, dried over anhydrous Na₂SO₄, filtered, and concentrated under vacuum to obtain crude product. The crude product was purified by flash column chromatography (SiO₂, 230-400, 90% ethyl acetate in petroleum ether) to afford tert-butyl 4-(1-(3-(2,6-bis(benzyloxy)pyridin-3-yl)-1-methyl-1H-indazol-7-yl) azetidin-3-yl)piperazine-1-carboxylate (0.49 g) as a yellow gummy solid.

LC-MS (ESI): m/z=661.69 [M+H]⁺.

### Preparation of tert-butyl 4-(1-(3-(2,6-dioxopiperidin-3-yl)-1-methyl-1H-indazol-7-yl) azetidin-3-yl)piperazine-1-carboxylate

To a stirred solution of tert-butyl 4-(1-(3-(2,6-bis(benzyloxy)pyridin-3-yl)-1-methyl-1H-indazol-7-yl) azetidin-3-yl)piperazine-1-carboxylate (0.4 g, 0.60 mmol) and acetic acid (0.6 mL) in THF (10 mL)/DMF (0.6 mL), 20% Pd(OH)₂ on carbon (0.510 g, 3.63 mmol) was added. The reaction was put under hydrogen atmosphere (15 psi) and stirred at room temperature for 16 hours. The reaction mixture was diluted with DCM and filtered through a pad of celite. The celite was washed with 30% THF in DCM. The filtrate was concentrated under vacuum to afford tert-butyl 4-(1-(3-(2,6-dioxopiperidin-3-yl)-1-methyl-1H-indazol-7-yl) azetidin-3-yl)piperazine-1-carboxylate (0.30 g) as a light yellow gummy solid. LC-MS (ESI): m/z=483.55 [M+H]⁺.

### Preparation of 3-(1-methyl-7-(3-(piperazin-1-yl) azetidin-1-yl)-1H-indazol-3-yl)piperidine-2,6-dione

To a stirred solution of tert-butyl 4-(1-(3-(2,6-dioxopiperidin-3-yl)-1-methyl-1H-indazol-7-yl) azetidin-3-yl)piperazine-1-carboxylate (0.3 g, 0.62 mmol) in DCM (3.5 mL), 4 M HCl in dioxane (3.5 mL) was added. The reaction was stirred at room temperature for 2 hours. The reaction mixture was concentrated under vacuum to obtain 3-(1-methyl-7-(3-(piperazin-1-yl) azetidin-1-yl)-1H-indazol-3-yl)piperidine-2,6-dione (0.30 g) as a pale yellow gummy solid.

LC-MS (ESI): m/z=383.43 [M+H]⁺.

### Preparation of 2-((6-((5-chloro-2-(4-(1-(3-(2,6-dioxopiperidin-3-yl)-1-methyl-1H-indazol-7-yl) azetidin-3-yl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-7-(3-(piperazin-1-yl) azetidin-1-yl)-1H-indazol-3-yl)piperidine-2,6-dione (0.3 g, 0.78 mmol) in DMSO (6 mL), 2-((6-((2,5-dichloropyrimidin-4-yl)amino)-1-methyl-2-oxo-1,2-dihydroquinolin-3-yl)oxy)-N-methylacetamide (0.09 g, 0.23 mmol) and DIPEA (1.09 mL, 6.27 mmol) was added. The reaction was heated to 100° C. for 6 hours. The reaction mixture was quenched with ice-cold water. The resulting solid precipitate was filtered and dried under vacuum to obtain crude product. The crude product was purified by preparative HPLC to afford 2-((6-((5-chloro-2-(4-(1-(3-(2,6-dioxopiperidin-3-yl)-1-methyl-1H-indazol-7-yl) azetidin-3-yl)piperazin-1-yl)pyrimidin-4-yl)amino)-1-methyl-2-oxo-1,2-dihydroquinolin-3-yl)oxy)-N-methylacetamide (0.029 g) as a pale brown solid.

LC-MS (ESI): m/z=752.61 [M−H]⁻.

¹H NMR (400 MHZ, DMSO-d₆): δ 10.87 (s, 1H), 8.88 (s, 1H), 8.07 (s, 1H), 7.95-7.92 (m, 2H), 7.76-7.73 (m, 1H), 7.47 (d, J=8.8 Hz, 1H), 7.24 (d, J=8.0 Hz, 1H), 7.12 (s, 1H), 7.02-6.98 (m, 1H), 6.76 (d, J=6.8 Hz, 1H), 4.58 (s, 2H), 4.32-4.31 (m, 1H), 4.18 (s, 3H), 4.01 (s, 2H), 3.85-3.51 (s, 9H), 3.25-3.30 (m, 1H), 2.65-2.57 (m, 5H), 2.40 (s, 4H), 2.33-2.29 (m, 1H), 2.29-2.13 (m, 1H).

### Example 24. Synthesis of 2-((6-((5-chloro-2-(4-(2-((4-(2,6-dioxopiperidin-3-yl)phenyl)amino)ethyl)piperidin-1-yl)pyrimidin-4-yl)amino)-1-methyl-2-oxo-1,2-dihydroquinolin-3-yl)oxy)-N-methylacetamide (Compound 284)

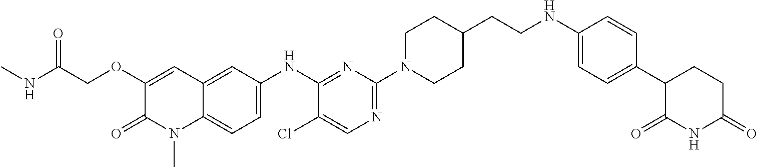 [SMILES: CNC(=O)COc1cc2cc(Nc3nc(N4CCC(CCNc5ccc(C6CCC(=O)NC6=O)cc5)CC4)ncc3Cl)ccc2n(C)c1=O]

### Preparation of tert-butyl 4-(2-((4-(2,6-bis(benzyloxy)pyridin-3-yl)phenyl)amino)ethyl)piperidine-1-carboxylate

A solution of tert-butyl 4-(2-aminoethyl)piperidine-1-carboxylate (0.38 g, 1.68 mmol), 2,6-bis(benzyloxy)-3-(4-bromophenyl)pyridine (0.75 g, 1.68 mmol), and potassium tert-butoxide (0.56 mL, 0.084 mmol) in 1,4-dioxane (15 mL) was degassed with nitrogen for 15 minutes. Bis(tri-tert-butylphosphine)palladium (0) (0.04 g, 0.08 mmol) was added. The resulting mixture was degassed for 10 minutes and heated with stirring at 80° C. for 24 hours. The reaction mixture was filtered through a pad of celite and concentrated under reduced pressure to obtain crude product. The crude product was purified by flash column (SiO₂, mesh size: 100-200, 25% EtOAc in petroleum ether) to afford tert-butyl 4-(2-((4-(2,6-bis(benzyloxy)pyridin-3-yl)phenyl)amino)ethyl)piperidine-1-carboxylate (570 mg) as a white solid.

LC-MS (ESI): m/z=594.65 [M+H]⁺.

### Preparation of tert-butyl 4-(2-((4-(2,6-dioxopiperidin-3-yl)phenyl)amino)ethyl)piperidine-1-carboxylate

To stirred solution of tert-butyl 4-(2-((4-(2,6-bis(benzyloxy)pyridin-3-yl)phenyl)amino)ethyl)piperidine-1-carboxylate (0.57 g, 0.96 mmol) in THF (34 mL), DMF (3 mL), 1,4-dioxane (6 mL), and acetic acid (0.5 mL) were added. 20% Palladium hydroxide (1.07 g) was added. The reaction was put under hydrogen atmosphere (balloon pressure) and stirred at room temperature for 3 hours. The reaction mixture was filtered through a pad of celite, and the filtrate was concentrated under reduced pressure to obtain crude product. The crude product was purified by flash column chromatography (SiO₂, mesh size 100-200, 60% EtOAc in petroleum ether) to afford tert-butyl 4-(2-((4-(2,6-dioxopiperidin-3-yl)phenyl)amino)ethyl)piperidine-1-carboxylate (0.300 g).

LC-MS (ESI): m/z=438.15 [M+23]⁺.

### Preparation of 3-(4-((2-(piperidin-4-yl)ethyl)amino)phenyl)piperidine-2,6-dione

To a stirred suspension of tert-butyl 4-(2-((4-(2,6-dioxopiperidin-3-yl)phenyl)amino)ethyl)piperidine-1-carboxylate (0.3 g, 0.72 mmol) in DCM (6 mL), hydrochloric acid (4 N in 1,4 dioxane)(3 mL) was added at 0° C. The reaction was allowed to warm to room temperature over 3 hours with stirring. Solvent was removed with reduced pressure to obtain 3-(4-((2-(piperidin-4-yl)ethyl)amino)phenyl)piperidine-2,6-dione (0.21 g).

LC-MS (ESI): m/z=316.17 [M+H]⁺.

### Preparation of 2-((6-((5-chloro-2-(4-(2-((4-(2,6-dioxopiperidin-3-yl)phenyl)amino)ethyl)piperidin-1-yl)pyrimidin-4-yl)amino)-1-methyl-2-oxo-1,2-dihydroquinolin-3-yl)oxy)-N-methylacetamide

To a stirred solution 3-(4-((2-(piperidin-4-yl)ethyl)amino)phenyl)piperidine-2,6-dione (0.21 g, 0.66 mmol, 1 eq.) and 2-((6-((2,5-dichloropyrimidin-4-yl)amino)-1-methyl-2-oxo-1,2-dihydroquinolin-3-yl)oxy)-N-methylacetamide (0.19 g, 0.46 mmol, 0.7 eq.) in dimethyl sulfoxide (4 mL), N,N-diisopropylethylamine (0.5 mL, 3.32 mmol, 5 eq.) was added at room temperature. The resulting reaction mixture was stirred at 100° C. for 3 hours. Water was added to the reaction, and the resulting solid was filtered to obtain crude product. The crude product was purified via prep-HPLC to afford 2-((6-((5-chloro-2-(4-(2-((4-(2,6-dioxopiperidin-3-yl)phenyl)amino)ethyl)piperidin-1-yl)pyrimidin-4-yl)amino)-1-methyl-2-oxo-1,2-dihydroquinolin-3-yl)oxy)-N-methylacetamide (0.028 g).

LC-MS (ESI): m/z=687.51 [M+H]⁺.

¹H NMR (400 MHZ, CDCl₃): δ 8.82 (s, 1H), 8.03 (s, 1H), 7.96-7.95 (m, 2H), 7.74-7.75 (m, 1H), 7.47 (d, J=9.20 Hz, 1H), 7.10 (s, 1H), 6.89 (d, J=8.40 Hz, 2H), 6.51 (d, J=8.40 Hz, 2H), 5.47 (t, J=4.80 Hz, 1H), 4.57 (s, 2H), 4.49 (d, J=12.40 Hz, 2H), 3.61-3.62 (m, 4H), 3.18 (d, 2H), 2.90 (t, J=12.40 Hz, 2H), 2.56-2.57 (m, 4H), 2.41-2.43 (m, 1H), 1.98-2.00 (m, 2H), 1.72 (t, J=20.00 Hz, 4H), 1.49 (d, J=6.80 Hz, 2H), 1.24 (s, 1H), 1.09 (d, J=9.60 Hz, 2H).

### Example 25. Synthesis of 2-((6-((5-chloro-2-(4-(1-(3-(2,6-dioxopiperidin-3-yl)-1-methyl-1H-indazol-7-yl) azetidin-3-yl)piperidin-1-yl)pyrimidin-4-yl)amino)-1-methyl-2-oxo-1,2-dihydroquinolin-3-yl)oxy)-N-methylacetamide (Compound 286)

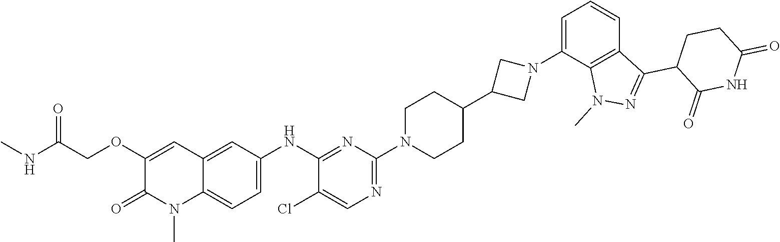 [SMILES: CNC(=O)COc1cc2cc(Nc3nc(N4CCC(C5CN(c6cccc7c(C8CCC(=O)NC8=O)nn(C)c67)C5)CC4)ncc3Cl)ccc2n(C)c1=O]

### Preparation of tert-butyl 4-(1-(3-(2,6-bis(benzyloxy)pyridin-3-yl)-1-methyl-1H-indazol-7-yl) azetidin-3-yl)piperidine-1-carboxylate

A solution of tert-butyl 4-(azetidin-3-yl)piperidine-1-carboxylate (0.30 g, 1.24 mmol), 3-(2,6-bis(benzyloxy)pyridin-3-yl)-7-bromo-1-methyl-1H-indazole (0.62 g, 1.24 mmol), and potassium tert-butoxide (0.42 g, 3.74 mmol) in 1,4-dioxane (6 mL) was degassed with argon for 5 minutes. Bis(tri-tert-butylphosphine)palladium (0) (0.06 g, 0.12 mmol) was added, and the reaction was degassed for 5 minutes. The reaction mixture was heated at 100° C. for 4 hours. The reaction mixture was filtered through a pad of celite and concentrated to afford crude product. The crude product was purified by column chromatography (SiO₂, 230-400 mesh, product was eluted at 40% ethyl acetate and petroleum ether) to afford tert-butyl 4-(1-(3-(2,6-bis(benzyloxy)pyridin-3-yl)-1-methyl-1H-indazol-7-yl) azetidin-3-yl)piperidine-1-carboxylate (0.70 g) as a brown solid.

LC-MS (ESI): m/z=660.72 [M+H]⁺.

### Preparation of tert-butyl 4-(1-(3-(2,6-dioxopiperidin-3-yl)-1-methyl-1H-indazol-7-yl) azetidin-3-yl)piperidine-1-carboxylate

A stirred solution of tert-butyl 4-(1-(3-(2,6-bis(benzyloxy)pyridin-3-yl)-1-methyl-1H-indazol-7-yl) azetidin-3-yl)piperidine-1-carboxylate (0.7 g, 1.06 mmol) in THF (14 mL) was degassed with nitrogen and 20% palladium hydroxide on carbon (1.4 g) was added to the reaction mixture. The reaction was put under hydrogen atmosphere (20 psi) and stirred at room temperature for 8 hours. The reaction mixture was filtered through a pad of celite. The celite was washed with THE, and the filtrate was concentrated to obtain crude product. The crude product was washed with n-pentane and dried to afford tert-butyl 4-(1-(3-(2,6-dioxopiperidin-3-yl)-1-methyl-1H-indazol-7-yl) azetidin-3-yl)piperidine-1-carboxylate (0.80 g) as a brown liquid.

LC-MS (ESI): m/z=382.45 [M-100+H]⁺.

### Preparation of 3-(1-methyl-7-(3-(piperidin-4-yl) azetidin-1-yl)-1H-indazol-3-yl)piperidine-2,6-dione

A stirred solution of tert-butyl 4-(1-(3-(2,6-dioxopiperidin-3-yl)-1-methyl-1H-indazol-7-yl) azetidin-3-yl)piperidine-1-carboxylate (0.4 g, 0.831 mmol) in DCM (4.0 mL) was cooled to 0° C. TFA (2 mL) was added, and the reaction mixture was allowed to warm to room temperature over 2 hours with stirring. The reaction mixture was concentrated to obtain 3-(1-methyl-7-(3-(piperidin-4-yl) azetidin-1-yl)-1H-indazol-3-yl)piperidine-2,6-dione (0.30 g).

LC-MS (ESI): m/z=382.51 [M+H]⁺.

### Preparation of 2-((6-((5-chloro-2-(4-(1-(3-(2,6-dioxopiperidin-3-yl)-1-methyl-1H-indazol-7-yl) azetidin-3-yl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-7-(3-(piperidin-4-yl) azetidin-1-yl)-1H-indazol-3-yl)piperidine-2,6-dione (0.30 g, 0.78 mmol) and 2-((6-((2,5-dichloropyrimidin-4-yl)amino)-1-methyl-2-oxo-1,2-dihydroquinolin-3-yl)oxy)-N-methylacetamide (0.32 g, 0.78 mmol) in DMSO (6 mL), N,N-diisopropylethylamine (1.36 mL, 7.86 mmol) was added. The reaction mixture was stirred at 100° C. for 4 hours. The reaction mixture was poured into ice-cold water and stirred for 15 minutes. The resulting precipitate was filtered and dried to obtain crude product. The crude product was purified by prep-HPLC to afford 2-((6-((5-chloro-2-(4-(1-(3-(2,6-dioxopiperidin-3-yl)-1-methyl-1H-indazol-7-yl) azetidin-3-yl)piperidin-1-yl)pyrimidin-4-yl)amino)-1-methyl-2-oxo-1,2-dihydroquinolin-3-yl)oxy)-N-methylacetamide (0.035 g) as an off-white solid.

LC-MS (ESI): m/z=753.53 [M+H]⁺.

¹H NMR (400 MHZ, DMSO-d₆): δ 10.87 (s, 1H), 8.84 (s, 1H), 8.04 (s, 1H), 7.96-7.94 (m, 2H), 7.75 (dd, J=2.40, 9.00 Hz, 1H), 7.47 (d, J=9.20 Hz, 1H), 7.21 (d, J=8.00 Hz, 1H), 7.09 (s, 1H), 6.98 (t, J=7.60 Hz, 1H), 6.73 (d, J=7.60 Hz, 1H), 4.57-4.51 (m, 4H), 4.32-4.29 (m, 1H), 4.18 (s, 3H), 3.96-3.94 (m, 2H), 3.67-3.59 (m, 5H), 2.85 (t, J=12.00 Hz, 2H), 2.65-2.59 (m, 5H), 2.42-2.18 (m, 2H), 1.76-1.73 (m, 3H), 1.36 (s, 3H), 1.23 (s, 1H), 1.10-1.04 (m, 2H).

### Example 26. Synthesis of 2-((6-((5-chloro-2-(4-((1-(3-(2,6-dioxopiperidin-3-yl)-1-methyl-1H-indazol-6-yl)piperidin-4-yl)oxy)piperidin-1-yl)pyrimidin-4-yl)amino)-1-methyl-2-oxo-1,2-dihydroquinolin-3-yl)oxy)-N-methylacetamide (Compound 287)

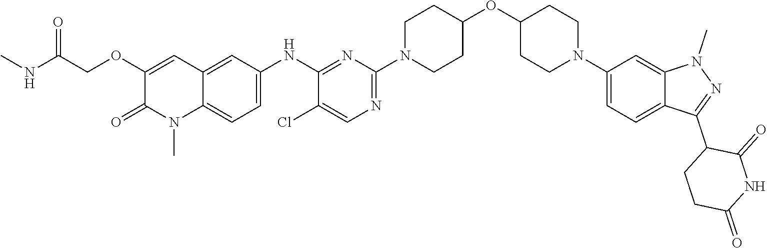 [SMILES: CNC(=O)COc1cc2cc(Nc3nc(N4CCC(OC5CCN(c6ccc7c(C8CCC(=O)NC8=O)nn(C)c7c6)CC5)CC4)ncc3Cl)ccc2n(C)c1=O]

### Preparation of tert-butyl 4-((1-(3-(2,6-bis(benzyloxy)pyridin-3-yl)-1-methyl-1H-indazol-6-yl)piperidin-4-yl)oxy)piperidine-1-carboxylate

A solution of 3-(2,6-bis(benzyloxy)pyridin-3-yl)-6-bromo-1-methyl-1H-indazole (0.65 g, 1.2 mmol), tert-butyl 4-(piperidin-4-yloxy)piperidine-1-carboxylate (0.443 g, 1.55 mmol), and potassium tert-butoxide (0.43 g, 3.8 mmol) in 1,4-dioxane (13 mL) was degassed with argon for 5 minutes. BrettPhos Pd G3 (0.117 g, 0.1 mmol) was added, and the reaction was degassed with argon for another 5 minutes. The reaction was heated at 90° C. for 3 hours. The reaction was quenched with water and extracted into ethyl acetate. The combined organic layers were washed with brine, dried over anhydrous Na₂SO₄, filtered, and concentrated under vacuum to obtain crude product. The crude purified by flash column chromatography (SiO₂, 60-120, 30% ethyl acetate in petroleum ether) to afford tert-butyl 4-((1-(3-(2,6-bis(benzyloxy)pyridin-3-yl)-1-methyl-1H-indazol-6-yl)piperidin-4-yl)oxy)piperidine-1-carboxylate (0.55 g) as a brown solid.

LC-MS (ESI): m/z=704.44 [M+H]⁺.

### Preparation of tert-butyl 4-((1-(3-(2,6-dioxopiperidin-3-yl)-1-methyl-1H-indazol-6-yl)piperidin-4-yl)oxy)piperidine-1-carboxylate

To a stirred solution of tert-butyl 4-((1-(3-(2,6-bis(benzyloxy)pyridin-3-yl)-1-methyl-1H-indazol-6-yl)piperidin-4-yl)oxy)piperidine-1-carboxylate (0.50 g, 0.7 mmol) in THF (15 mL), 20% Pd(OH)₂ on carbon (1.0 g) was added. The reaction was put under hydrogen atmosphere (balloon pressure) and stirred at room temperature for 3 hours. The reaction mixture was filtered through a pad of celite. The celite was washed with THF. The filtrate was concentrated to obtain crude product. The crude product was triturated with n-pentane and dried to afford tert-butyl 4-((1-(3-(2,6-dioxopiperidin-3-yl)-1-methyl-1H-indazol-6-yl)piperidin-4-yl)oxy)piperidine-1-carboxylate (0.35 g) as a brown solid.

LC-MS (ESI): m/z=526.61 [M+H]⁺.

### Preparation of 3-(1-methyl-6-(4-(piperidin-4-yloxy)piperidin-1-yl)-1H-indazol-3-yl)piperidine-2,6-dione

A stirred solution of tert-butyl 4-((1-(3-(2,6-dioxopiperidin-3-yl)-1-methyl-1H-indazol-6-yl)piperidin-4-yl)oxy)piperidine-1-carboxylate (0.30 g, 0.50 mmol) in DCM (6 mL) was cooled to 0° C. 4 M HCl in 1,4-dioxane (3 mL) was added, and the reaction was allowed to warm to room temperature over 3 hours with stirring. The solvent was evaporated to dryness to obtain crude product. The crude product was triturated with diethyl ether (40 mL) to afford 3-(1-methyl-6-(4-(piperidin-4-yloxy)piperidin-1-yl)-1H-indazol-3-yl)piperidine-2,6-dione (0.29 g) as an off-white gummy semi-solid.

LC-MS (ESI): m/z=426.48 [M+H]⁺.

### Preparation of 2-((6-((5-chloro-2-(4-((1-(3-(2,6-dioxopiperidin-3-yl)-1-methyl-1H-indazol-6-yl)piperidin-4-yl)oxy)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(4-(piperidin-4-yloxy)piperidin-1-yl)-1H-indazol-3-yl)piperidine-2,6-dione hydrochloride (0.27 g, 0.58 mmol) and 2-((6-((2,5-dichloropyrimidin-4-yl)amino)-1-methyl-2-oxo-1,2-dihydroquinolin-3-yl)oxy)-N-methyl acetamide (0.095 g, 0.23 mmol) in DMSO, DIPEA (0.605 g, 4.68 mmol) was added. The reaction mixture was stirred at 120° C. for 3 hours. The reaction was quenched in ice-cold water and stirred for 15 minutes. The resulting precipitate was filtered and dried to obtain crude product. The crude product was purified by prep-HPLC to afford 2-((6-((5-chloro-2-(4-((1-(3-(2,6-dioxopiperidin-3-yl)-1-methyl-1H-indazol-6-yl)piperidin-4-yl)oxy)piperidin-1-yl)pyrimidin-4-yl)amino)-1-methyl-2-oxo-1,2-dihydroquinolin-3-yl)oxy)-N-methylacetamide (0.038 g) as an off-white solid.

LC-MS (ESI): m/z=795.63 [M+H]⁺.

¹H NMR (400 MHZ, DMSO-d₆): δ 10.85 (s, 1H), 8.86 (s, 1H), 8.44 (s, 1H), 8.05 (s, 1H), 7.96 (q, J=4.0 Hz, 2H), 7.75 (q, J=3.8 Hz, 1H), 7.48 (d, J=8.7 Hz, 2H), 7.11 (s, 1H), 6.91 (q, J=3.5 Hz, 1H), 6.85 (s, 1H), 4.58 (s, 1H), 4.25 (q, J=4.8 Hz, 1H), 4.09 (d, J=13.6 Hz, 1H), 3.89 (s, 1H), 3.68 (s, 1H), 3.60 (d, J=12.6 Hz, 1H), 2.98 (t, J=9.9 Hz, 1H), 2.64 (t, J=12.4 Hz, 1H), 2.31 (m, J=3.9 Hz, 1H), 2.16 (q, J=6.3 Hz, 1H), 1.95 (d, J=10.0 Hz, 1H), 1.85 (d, J=10.6 Hz, 1H), 1.58 (q, J=9.2 Hz, 1H), 1.42 (d, J=8.8 Hz, 1H).

### Example 27. Synthesis of 2-((6-((5-chloro-2-(4-((1-(3-(2,6-dioxopiperidin-3-yl)-1-methyl-1H-indazol-6-yl)piperidin-4-yl)methyl)piperidin-1-yl)pyrimidin-4-yl)amino)-1-methyl-2-oxo-1,2-dihydroquinolin-3-yl)oxy)-N-methylacetamide (Compound 288)

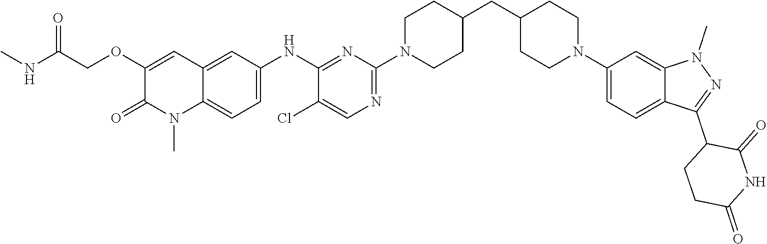 [SMILES: CNC(=O)COc1cc2cc(Nc3nc(N4CCC(CC5CCN(c6ccc7c(C8CCC(=O)NC8=O)nn(C)c7c6)CC5)CC4)ncc3Cl)ccc2n(C)c1=O]

### Preparation of tert-butyl 4-((1-(3-(2,6-bis(benzyloxy)pyridin-3-yl)-1-methyl-1H-indazol-6-yl)piperidin-4-yl)methyl)piperidine-1-carboxylate

A stirred solution of tert-butyl 4-(piperidin-4-ylmethyl)piperidine-1-carboxylate (0.4 g, 1.41 mmol), 3-(2,6-bis(benzyloxy)pyridin-3-yl)-6-bromo-1-methyl-1H-indazole (0.70 g, 1.41 mmol), and sodium tert-butoxide (0.40 g, 4.24 mmol) in 1,4-dioxane (8 mL) was degassed with nitrogen for 15 minutes. BrettPhos Pd G3 (0.96 g, 0.14 mmol) was added. The resulting mixture was degassed again for 10 minutes with nitrogen. The reaction was heated with stirring at 80° C. for 6 hours. The reaction mixture was filtered through a pad of celite, and the filtrate was concentrated under reduced pressure to obtain crude product. The crude product was adsorbed on silica gel and purified by flash column chromatography (silica gel; 12% ethyl acetate in petroleum ether) to afford tert-butyl 4-((1-(3-(2,6-bis(benzyloxy)pyridin-3-yl)-1-methyl-1H-indazol-6-yl)piperidin-4-yl)methyl)piperidine-1-carboxylate (0.38 g) as a brown semi-solid.

LC-MS (ESI): m/z=540.62 [M+H]⁺.

### Preparation of tert-butyl 4-((1-(3-(2,6-dioxopiperidin-3-yl)-1-methyl-1H-indazol-6-yl)piperidin-4-yl)methyl)piperidine-1-carboxylate

To stirred solution of tert-butyl 4-((1-(3-(2,6-bis(benzyloxy)pyridin-3-yl)-1-methyl-1H-indazol-6-yl)piperidin-4-yl)methyl)piperidine-1-carboxylate (0.38 g, 0.54 mmol) in ethyl acetate (11.4 mL), palladium on activated carbon (0.38 g) was added. The reaction was put under hydrogen atmosphere (balloon pressure) for 12 hours at room temperature with stirring. The reaction mixture was filtered through a pad of celite, and the filtrate was concentrated under reduced pressure to obtain crude product. The crude product was purified by flash column chromatography (SiO₂, 60-120, 25% ethyl acetate in petroleum ether) to afford tert-butyl 4-((1-(3-(2,6-dioxopiperidin-3-yl)-1-methyl-1H-indazol-6-yl)piperidin-4-yl)methyl)piperidine-1-carboxylate (0.42 g) as an off-white solid.

LC-MS (ESI): m/z=362.40 [M+H]⁺.

### Preparation of 3-(1-methyl-6-(4-(piperidin-4-ylmethyl)piperidin-1-yl)-1H-indazol-3-yl)piperidine-2,6-dione hydrochloride

To a stirred solution of tert-butyl 4-((1-(3-(2,6-dioxopiperidin-3-yl)-1-methyl-1H-indazol-6-yl)piperidin-4-yl)methyl)piperidine-1-carboxylate (0.42 g, 1.16 mmol) in DCM (5 mL), 4 M HCl in 1,4-dioxane (2.2 mL) was added dropwise the reaction. The reaction was stirred at room temperature for 16 hours. The reaction mixture was concentrated via rotary evaporator to obtain crude product. The crude product was triturated with diethyl ether to afford 3-(1-methyl-6-(4-(piperidin-4-ylmethyl)piperidin-1-yl)-1H-indazol-3-yl)piperidine-2,6-dione hydrochloride (0.21 g) as a white solid.

LC-MS (ESI): m/z=262.25 [M+H]⁺.

### Preparation of 2-((6-((5-chloro-2-(4-((1-(3-(2,6-dioxopiperidin-3-yl)-1-methyl-1H-indazol-6-yl)piperidin-4-yl)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(4-(piperidin-4-ylmethyl)piperidin-1-yl)-1H-indazol-3-yl)piperidine-2,6-dione hydrochloride (0.21 g, 0.45 mmol) in DMSO (6.3 mL), DIPEA (0.8 mL, 4.57 mmol) and p-toluenesulfonic acid (0.004 g, 0.023 mmol) were added. The reaction was degassed with nitrogen for 1 minute. 2-((6-((2,5-dichloropyrimidin-4-yl)amino)-1-methyl-2-oxo-1,2-dihydroquinolin-3-yl)oxy)-N-methylacetamide (0.13 g, 0.32 mmol) was added, and the reaction was heated to 165° C. for 3 hours. The reaction was quenched with water. The resulting precipitate was filtered and dried under vacuum to obtain crude product. The crude product was purified by prep-HPLC to afford 2-((6-((5-chloro-2-(4-((1-(3-(2,6-dioxopiperidin-3-yl)-1-methyl-1H-indazol-6-yl)piperidin-4-yl)methyl)piperidin-1-yl)pyrimidin-4-yl)amino)-1-methyl-2-oxo-1,2-dihydroquinolin-3-yl)oxy)-N-methylacetamide (0.027 g) as a white solid.

LC-MS (ESI): m/z=534.54 [M+H]⁺.

¹H NMR (400 MHZ, DMSO-d₆): δ 10.84 (s, 1H), 8.82 (s, 1H), 8.03 (s, 1H), 7.95 (t, J=5.0 Hz, 2H), 7.75 (q, J=3.8 Hz, 1H), 7.47 (t, J=5.3 Hz, 2H), 7.09 (s, 1H), 6.91 (s, 1H), 6.81 (s, 1H), 4.58 (s, 1H), 4.24 (q, J=4.7 Hz, 1H), 3.88 (s, 3H), 3.77 (d, J=11.8 Hz, 2H), 3.68 (s, 3H), 2.84 (t, J=11.9 Hz, 2H), 2.4-2.68 (m, J=13.4 Hz, 7H), 2.33 (m, 1H), 2.16 (m, 1H), 1.72 (m, 7H), 1.23 (m, 5H), 1.05 (m, 2H).

### Example 28. Synthesis of 2-((6-((5-chloro-2-((3R)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3R)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide] (Compound 290a)

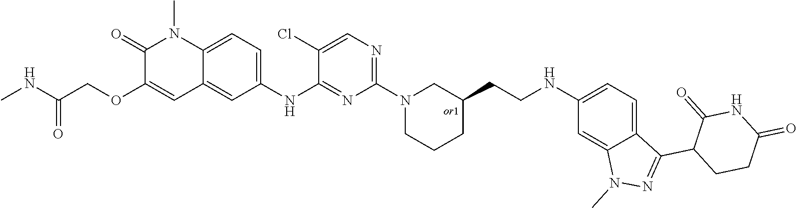 [SMILES: CNC(=O)COc1cc2cc(Nc3nc(N4CCC[C@H](CCNc5ccc6c(C7CCC(=O)NC7=O)nn(C)c6c5)C4)ncc3Cl)ccc2n(C)c1=O]

### Preparation of tert-butyl 3-(2-((3-(2,6-bis(benzyloxy)pyridin-3-yl)-1-methyl-1H-indazol-6-yl)amino)ethyl)piperidine-1-carboxylate (Isomer 1 and 2

To stirred solution of 3-(2,6-bis(benzyloxy)pyridin-3-yl)-6-bromo-1-methyl-1H-indazole (1.0 g, 1.99 mmol) and tert-butyl 3-(2-aminoethyl)piperidine-1-carboxylate (0.45 g, 1.99 mmol) in 1,4-dioxane (10 mL), sodium tert-butoxide (0.57 g, 5.99 mmol) was added. The reaction was degassed for 15 minutes with nitrogen. BrettPhos-Pd-G3 (0.09 g, 0.1 mmol) was added, and the reaction was stirred at 100° C. for 6 hours. The reaction mixture was cooled to room temperature and filtered through a pad of celite. The filtrate was concentrated to obtain crude product. The crude product was purified by flash column chromatography (SiO₂, 230-400, 15% ethyl acetate in petroleum ether) to afford tert-butyl 3-(2-((3-(2,6-bis(benzyloxy)pyridin-3-yl)-1-methyl-1H-indazol-6-yl)amino)ethyl)piperidine-1-carboxylate (1.0 g). The racemic mixture was subjected to SFC separation (Column: (R, R) Whelk-01 (4.6×250 mm, internal diameter: 5 μm); mobile phase: Phase A (CO₂), and Phase B (MeCN/MeOH with 0.2% NH₃·H₂O); isocratic elution B %: 40%; flow rate: 3.0 mL/min; detector: PDA; wavelength: 215 nm; column temperature: 30° C.; back pressure: 100 bar) to afford tert-butyl 3-(2-((3-(2,6-bis(benzyloxy)pyridin-3-yl)-1-methyl-1H-indazol-6-yl)amino)ethyl piperidine-1-carboxylate (0.45 g)(Isomer 1, Rt=4.85 min) and tert-butyl 3-(2-((3-(2,6-bis(benzyloxy)pyridin-3-yl)-1-methyl-1H-indazol-6-yl)amino)ethyl)piperidine-1-carboxylate (0.5 g)(Isomer 2, Rt=8.18 min).

LC-MS (ESI): m/z=647.34 [M+H]⁺.

### Preparation of tert-butyl 3-(2-((3-(2,6-dioxopiperidin-3-yl)-1-methyl-1H-indazol-6-yl)amino)ethyl)piperidine-1-carboxylate (Isomer 1)

To a stirred solution of tert-butyl 3-(2-((3-(6-(benzyloxy)-2-hydroxypyridin-3-yl)-1-methyl-1H-indazol-6-yl)amino)ethyl)piperidine-1-carboxylate (Isomer 1) (0.45 g, 0.69 mmol) in THF (4.5 mL), 20% Pd(OH)₂ (0.46 g, 3.45 mmol) was added. The reaction was put under hydrogen atmosphere (15 psi) and stirred for 4 hours at room temperature. The reaction mixture was diluted with THF (20 mL) and filtered through a pad of celite. The filtrate was collected and concentrated under vacuum to afford tert-butyl 3-(2-((3-(2,6-dioxopiperidin-3-yl)-1-methyl-1H-indazol-6-yl)amino)ethyl)piperidine-1-carboxylate (Isomer 1) (0.30 g) as a yellow semi-solid.

LC-MS (ESI): m/z=469.26 [M+H]⁺.

### Preparation of 3-(1-methyl-6-((2-(piperidin-3-yl)ethyl)amino)-1H-indazol-3-yl)piperidine-2,6-dione (Isomer 1)

To a stirred solution of tert-butyl 3-(2-((3-(2,6-dioxopiperidin-3-yl)-1-methyl-1H-indazol-6-yl)amino)ethyl)piperidine-1-carboxylate (Isomer 1) (0.30 g, 0.63 mmol) in DCM (6 mL), TFA (3 mL) was added. The reaction was stirred for 3 hours at room temperature. The reaction mixture was concentrated and triturated with diethyl ether to afford 3-(1-methyl-6-((2-(piperidin-3-yl)ethyl)amino)-1H-indazol-3-yl)piperidine-2,6-dione (Isomer 1) (0.20 g) as the TFA salt.

LC-MS (ESI): m/z=369.22 [M+H]⁺.

### Preparation of 2-((6-((5-chloro-2-((3R)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3R)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide]

To a stirred solution of 3-(1-methyl-6-((2-(piperidin-3-yl)ethyl)amino)-1H-indazol-3-yl)piperidine-2,6-dione (TFA salt)(0.20 g, 0.54 mmol) and 2-((6-((2,5-dichloropyrimidin-4-yl)amino)-1-methyl-2-oxo-1,2-dihydroquinolin-3-yl)oxy)-N-methylacetamide (0.08 g, 0.218 mmol) in DMSO (2 mL), DIPEA (0.94 mL, 5.42 mmol) was added. The reaction was heated to 100° C. for 6 hours. The reaction mixture was quenched with ice-cold water. The resulting solid was filtered to obtain crude product. The crude product was purified by prep HPLC to afford 2-((6-((5-chloro-2-((3R)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide [rel-2-((6-((5-chloro-2-((3R)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide] (0.031 g) as an off-white solid.

LC-MS (ESI): m/z=739.53 [M−H]⁻.

¹H NMR (400 MHZ, DMSO-d₆): δ 10.80 (s, 1H), 8.82 (s, 1H), 8.04 (s, 1H), 7.89 (br s, 2H), 7.78 (d, J=9.20 Hz, 1H), 7.34-7.29 (m, 2H), 7.10 (s, 1H), 6.48 (d, J=8.8 Hz, 1H), 6.25 (s, 1H), 5.85 (s, 1H), 4.42 (s, 2H), 4.39-4.38 (m, 1H), 4.30-4.28 (m, 1H), 4.18-4.15 (m, 1H), 3.73 (s, 3H), 3.48-3.32 (m, 3H), 3.09-3.08 (m, 2H), 2.97-3.94 (m, 1H), 2.78-2.75 (m, 1H), 2.69 (d, J=3.60 Hz, 3H), 2.63-2.61 (m, 2H), 2.25-2.16 (m, 2H), 1.90-1.87 (m, 1H), 1.70-1.43 (m, 6H), 1.50 (d, J=7.6 Hz, 1H).

### Example 29. Synthesis of 2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide] (Compound 290b)

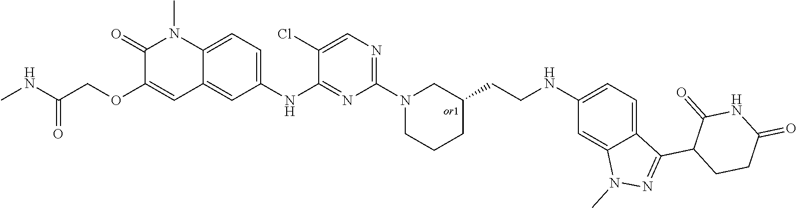 [SMILES: CNC(=O)COc1cc2cc(Nc3nc(N4CCC[C@@H](CCNc5ccc6c(C7CCC(=O)NC7=O)nn(C)c6c5)C4)ncc3Cl)ccc2n(C)c1=O]

### Preparation of tert-butyl 3-(2-((3-(2,6-dioxopiperidin-3-yl)-1-methyl-1H-indazol-6-yl)amino)ethyl)piperidine-1-carboxylate (Isomer 2)

To a stirred solution of tert-butyl 3-(2-((3-(2,6-bis(benzyloxy)pyridin-3-yl)-1-methyl-1H-indazol-6-yl)amino)ethyl)piperidine-1-carboxylate (Isomer 2) (0.50 g, 0.77 mmol) in THF (5 mL), 20% Pd(OH)₂ (0.54 g, 3.86 mmol) was added. The reaction was put under hydrogen atmosphere (15 psi) and stirred for 4 hours at room temperature. The reaction mixture was diluted with THF (20 mL) and filtered through a pad of celite. The filtrate was collected and concentrated under vacuum to afford tert-butyl 3-(2-((3-(2,6-dioxopiperidin-3-yl)-1-methyl-1H-indazol-6-yl)amino)ethyl)piperidine-1-carboxylate (Isomer 2) (0.31 g) as a brown semi-solid.

LC-MS (ESI): m/z=470.58 [M+H]⁺.

### Preparation of 3-(1-methyl-6-((2-(piperidin-3-yl)ethyl)amino)-1H-indazol-3-yl)piperidine-2,6-dione (Isomer 2)

To a stirred solution of tert-butyl 3-(2-((3-(2,6-dioxopiperidin-3-yl)-1-methyl-1H-indazol-6-yl)amino)ethyl)piperidine-1-carboxylate (Isomer 2) (0.30 g, 0.63 mmol) in DCM (3 mL), TFA (3 mL) was added. The reaction was stirred for 3 hours at room temperature. The reaction mixture was concentrated to obtain crude product. The crude product was triturated with diethyl ether to afford 3-(1-methyl-6-((2-(piperidin-3-yl)ethyl)amino)-1H-indazol-3-yl)piperidine-2,6-dione (Isomer 2) (0.20 g) as the TFA salt.

LC-MS (ESI): m/z=370.50 [M+H]⁺.

### Preparation of 2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide]

To a stirred solution of 3-(1-methyl-6-((2-(piperidin-3-yl)ethyl)amino)-1H-indazol-3-yl)piperidine-2,6-dione (TFA salt)(Isomer 2) (0.20 g, 0.54 mmol) and 2-((6-((2,5-dichloropyrimidin-4-yl)amino)-1-methyl-2-oxo-1,2-dihydroquinolin-3-yl)oxy)-N-methylacetamide (0.08 g, 0.21 mmol) in DMSO (2 mL), DIPEA (0.9 mL, 5.42 mmol) was added. The reaction was heated to 100° C. for 6 hours. The reaction was quenched with ice-cold water. The resulting solid was filtered to obtain crude product. The crude product was purified by prep-HPLC to afford 2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide [rel-2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide] (24 mg) as an off-white solid.

LC-MS (ESI): m/z=741.49 [M+H]⁺.

¹H NMR (400 MHZ, DMSO-d₆): δ 10.98 (s, 1H), 8.82 (s, 1H), 8.04 (s, 1H), 7.89 (s, 2H), 7.78 (d, J=9.20 Hz, 1H), 7.30-7.28 (m, 2H), 7.10 (s, 1H), 6.48 (d, J=8.40 Hz, 1H), 6.25 (s, 1H), 5.85 (s, 1H), 4.51 (s, 2H), 4.45-4.39 (m, 2H), 4.18-4.15 (m, 1H), 3.92-3.44 (m, 6H), 3.15-3.09 (m, 2H), 2.97-2.96 (m, 1H), 2.75-2.71 (m, 1H), 2.67-2.58 (m, 5H), 2.25-2.14 (m, 2H), 1.90-1.87 (m, 1H), 1.69-1.39 (m, 5H), 1.23 (br s, 1H).

### Example 30. Synthesis of 2-((6-((5-chloro-2-((2R)-2-(((3-(2,6-dioxopiperidin-3-yl)-1-methyl-1H-indazol-6-yl)amino)methyl)pyrrolidin-1-yl)pyrimidin-4-yl)amino)-1-methyl-2-oxo-1,2-dihydroquinolin-3-yl)oxy)-N-methylacetamide (Compound 279b)

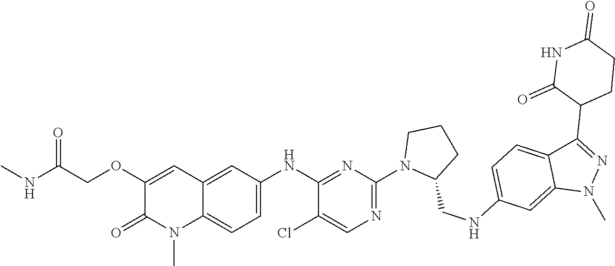 [SMILES: CNC(=O)COc1cc2cc(Nc3nc(N4CCC[C@@H]4CNc4ccc5c(C6CCC(=O)NC6=O)nn(C)c5c4)ncc3Cl)ccc2n(C)c1=O]

### Preparation of tert-butyl-(R)-2-(((3-(2,6-bis(benzyloxy)pyridin-3-yl)-1-methyl-1H-indazol-6-yl)amino)methyl)pyrrolidine-1-carboxylate

A solution of 3-(2,6-bis(benzyloxy)pyridin-3-yl)-6-bromo-1-methyl-1H-indazole (0.8 g, 1.6 mmol), tert-butyl (R)-2-(aminomethyl)pyrrolidine-1-carboxylate (0.38 g, 1.9 mmol), and sodium tertiary butoxide (0.46 g, 4.8 mmol) in 1,4-dioxane (8 mL) was degassed with argon for 5 minutes. BrettPhos Pd G3 (0.14 g, 0.1 mmol) was added, and the reaction was degassed for 5 minutes. The reaction mixture was heated at 100° C. for 4 hours. The reaction mixture was filtered through a pad of celite and concentrated to obtain crude product. The crude was purified by column chromatography (neutral alumina, product eluted at 2% methanol in dichloromethane) to afford tert-butyl-(R)-2-(((3-(2,6-bis(benzyloxy)pyridin-3-yl)-1-methyl-1H-indazol-6-yl)amino)methyl)pyrrolidine-1-carboxylate (0.80 g) as a brown liquid.

LC-MS (ESI): m/z=620.62 [M+H]⁺.

### Preparation of tert-butyl (2R)-2-(((3-(2,6-dioxopiperidin-3-yl)-1-methyl-1H-indazol-6-yl)amino)methyl)pyrrolidine-1-carboxylate

To a stirred solution of tert-butyl-(R)-2-(((3-(2,6-bis(benzyloxy)pyridin-3-yl)-1-methyl-1H-indazol-6-yl)amino)methyl)pyrrolidine-1-carboxylate (0.80 g, 1.13 mmol) in THF (48 mL), 20% Pd(OH)₂ on carbon (1.6 g) was added. The reaction was put under hydrogen atmosphere (15 psi) and stirred at room temperature for 4 hours. The reaction mixture was filtered through a pad of celite and concentrated. The crude product was washed with n-pentane and dried to afford tert-butyl (2R)-2-(((3-(2,6-dioxopiperidin-3-yl)-1-methyl-1H-indazol-6-yl)amino)methyl)pyrrolidine-1-carboxylate (0.55 g) as a brown solid.

LC-MS (ESI): m/z=442.48 [M+H]⁺.

### Preparation of 3-(1-methyl-6-((((R)-pyrrolidin-2-yl)methyl)amino)-1H-indazol-3-yl)piperidine-2,6-dione

A stirred solution of tert-butyl (2R)-2-(((3-(2,6-dioxopiperidin-3-yl)-1-methyl-1H-indazol-6-yl)amino)methyl)pyrrolidine-1-carboxylate (0.55 g, 1.24 mmol) in DCM (11 mL) was cooled to 0° C. TFA (5.5 mL) was added to the reaction. The reaction mixture was allowed to warm to room temperature over 4 hours with stirring. The reaction mixture was concentrated to obtain crude product. The crude product was washed with n-pentane to afford 3-(1-methyl-6-((((R)-pyrrolidin-2-yl)methyl)amino)-1H-indazol-3-yl)piperidine-2,6-dione (0.35 g) as a brown gummy solid.

LC-MS (ESI): m/z=342.40 [M+H]⁺.

### Preparation of 2-((6-((5-chloro-2-((2R)-2-(((3-(2,6-dioxopiperidin-3-yl)-1-methyl-1H-indazol-6-yl)amino)methyl)pyrrolidin-1-yl)pyrimidin-4-yl)amino)-1-methyl-2-oxo-1,2-dihydroquinolin-3-yl)oxy)-N-methylacetamide

To a stirred solution 3-(1-methyl-6-((((R)-pyrrolidin-2-yl)methyl)amino)-1H-indazol-3-yl)piperidine-2,6-dione (0.35 g, 1.02 mmol) and 2-((6-((2,5-dichloropyrimidin-4-yl)amino)-1-methyl-2-oxo-1,2-dihydroquinolin-3-yl)oxy)-N-methylacetamide (0.20 g, 0.51 mmol) in DMSO, N,N-diisopropylethylamine (1.05 g, 5.12 mmol) was added. The resulting reaction mixture was stirred at 100° C. for 6 hours. The reaction was poured into ice-cold water and stirred for 15 minutes. The resulting precipitate was filtered and dried to obtain crude product. The crude product was purified by prep-HPLC to afford 2-((6-((5-chloro-2-((2R)-2-(((3-(2,6-dioxopiperidin-3-yl)-1-methyl-1H-indazol-6-yl)amino)methyl)pyrrolidin-1-yl)pyrimidin-4-yl)amino)-1-methyl-2-oxo-1,2-dihydroquinolin-3-yl)oxy)-N-methylacetamide (0.071 g) as a brown solid.

LC-MS (ESI): m/z=713.46 [M+H]⁺.

¹H NMR (400 MHZ, DMSO-d₆): δ 10.81 (s, 1H), 8.83 (s, 1H), 8.16-7.80 (m, 4H), 7.48-5.83 (m, 6H), 4.58-4.50 (m, 2H), 4.36-4.17 (m, 2H), 3.83-3.34 (m, 8H), 3.01-2.99 (m, 2H), 2.67-2.54 (m, 7H), 2.28-2.16 (m, 2H), 2.07-1.90 (m, 4H), 1.27-1.22 (m, 1H).

### Example 31. Synthesis of 2-((6-((5-chloro-2-((((1r,4r)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide (Compound 289b)

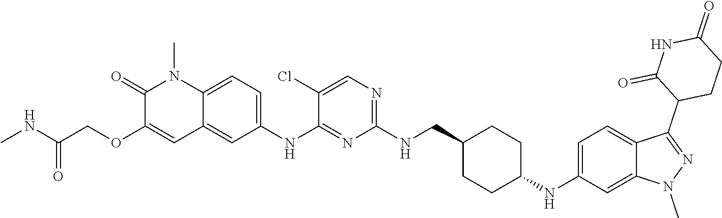 [SMILES: CNC(=O)COc1cc2cc(Nc3nc(NC[C@H]4CC[C@H](Nc5ccc6c(C7CCC(=O)NC7=O)nn(C)c6c5)CC4)ncc3Cl)ccc2n(C)c1=O]

### Preparation of tert-butyl (((1r,4r)-4-((3-(2,6-bis(benzyloxy)pyridin-3-yl)-1-methyl-1H-indazol-6-yl)amino)cyclohexyl)methyl) carbamate

To a stirred solution of tert-butyl (((1r,4r)-4-aminocyclohexyl)methyl) carbamate (0.500 g, 2.19 mmol) and 3-(2,6-bis(benzyloxy)pyridin-3-yl)-6-bromo-1-methyl-1H-indazole (0.76 g, 1.53 mmol) in 1,4-dioxane (5 mL), sodium tert-butoxide (0.420 g, 4.38 mmol) was added. The reaction mixture was purged N₂ for 5 minutes. t-BuBrettPhos Pd-G3 (0.93 g, 0.11 mmol) was added, and the reaction mixture was stirred at 100° C. for 16 hours. The reaction was quenched with ice-cold water (50 mL) and extracted with ethyl acetate (100 mL×2). The organic layer was washed with brine solution, dried over anhydrous sodium sulfate, filtered, and concentrated to obtain crude product. The crude product was purified by flash column chromatography (SiO₂, 100-200, 30% ethyl acetate in petroleum ether) to obtain tert-butyl (((1r,4r)-4-((3-(2,6-bis(benzyloxy)pyridin-3-yl)-1-methyl-1H-indazol-6-yl)amino)cyclohexyl)methyl) carbamate (0.86 g) as an off-white solid.

LC-MS (ESI): m/z=648.77 [M+H]⁺.

### Preparation of tert-butyl (((1r,4r)-4-((3-(2,6-dioxopiperidin-3-yl)-1-methyl-1H-indazol-6-yl)amino)cyclohexyl)methyl) carbamate

To stirred solution of tert-butyl (((1r,4r)-4-((3-(2,6-bis(benzyloxy)pyridin-3-yl)-1-methyl-1H-indazol-6-yl)amino)cyclohexyl)methyl) carbamate (0.50 g, 0.77 mmol) in THF (5 mL), palladium hydroxide on carbon was added. The reaction was put under hydrogen atmosphere (balloon pressure) and stirred at room temperature for 3 hours. The reaction mixture was filtered through a pad of celite, and the celite was washed with ethyl acetate. The filtrate was concentrated to obtain crude product. The crude product was triturated with n-hexane (2×100 mL) to afford tert-butyl (((1r,4r)-4-((3-(2,6-dioxopiperidin-3-yl)-1-methyl-1H-indazol-6-yl)amino)cyclohexyl)methyl) carbamate (0.23 g) as a light-brown solid.

LC-MS (ESI): m/z=470.59 [M+H]⁺.

### Preparation of 3-(6-(((1r,4r)-4-(aminomethyl)cyclohexyl)amino)-1-methyl-1H-indazol-3-yl)piperidine-2,6-dione hydrochloride

To a stirred solution of tert-butyl (((1r,4r)-4-((3-(2,6-dioxopiperidin-3-yl)-1-methyl-1H-indazol-6-yl)amino)cyclohexyl)methyl) carbamate (0.20 g, 0.43 mmol) in DCM (2 mL), 4 M HCl in 1,4-dioxane (1 mL) was added at 0° C. under a nitrogen atmosphere. The reaction was allowed to warm to room temperature over 3 hours with stirring. The reaction mixture was concentrated under reduced pressure to obtain crude product. The crude product was purified by trituration with diethyl ether and n-hexane to afford 3-(6-(((1r,4r)-4-(aminomethyl)cyclohexyl)amino)-1-methyl-1H-indazol-3-yl)piperidine-2,6-dione hydrochloride (0.13 g) as an off-white solid.

LC-MS (ESI): m/z=370.42 [M+H]⁺.

### Preparation of 2-((6-((5-chloro-2-((((1r,4r)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide

To a stirred solution of 3-(6-(((1r,4r)-4-(aminomethyl)cyclohexyl)amino)-1-methyl-1H-indazol-3-yl)piperidine-2,6-dione hydrochloride (0.13 g, 0.32 mmol) in DMSO (1.3 mL), DIPEA (0.16 mL, 0.96 mmol) was added at room temperature. After stirring for 10 minutes, 2-((6-((2,5-dichloropyrimidin-4-yl)amino)-1-methyl-2-oxo-1,2-dihydroquinolin-3-yl)oxy)-N-methylacetamide (0.13 g, 0.32 mmol) was added. The reaction was stirred at 100° C. for 16 hours. The reaction was quenched with ice-cold water (10 mL) and extracted with ethyl acetate (50 mL×2). The separated organic layers were washed with brine solution, dried over anhydrous sodium sulfate, filtered, and concentrated to obtain crude product. The crude product was purified by preparative HPLC to afford 2-((6-((5-chloro-2-((((1r,4r)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide (0.038 g) as a white solid.

LC-MS (ESI): m/z=741.49 [M+H]⁺.

¹H NMR (400 MHZ, DMSO-d₆): δ 10.81 (s, 1H), 8.71 (s, 1H), 8.22 (s, 1H), 8.05 (s, 1H), 7.96 (s, 1H), 7.84 (d, J=6.6 Hz, 1H), 7.45 (d, J=9.2 Hz, 1H), 7.24 (t, J=24.0 Hz, 1H), 6.47 (d, J=8.7 Hz, 1H), 6.34 (s, 1H), 5.64 (s, 1H), 4.57 (s, 1H), 4.16 (q, J=4.6 Hz, 1H), 3.79 (s, 1H), 3.67 (s, 1H), 3.10 (s, 1H), 2.66 (d, J=4.3 Hz, 1H), 2.59 (t, J=6.5 Hz, 1H), 2.20 (m, J=8.6 Hz, 1H), 2.05 (d, J=17.8 Hz, 1H), 1.77 (s, 1H), 1.55 (s, 1H), 1.07 (s, 1H).

### Example 32. Synthesis of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-isopropyl-2-oxo-1,2-dihydroquinolin-3-yl)oxy)-N-methylacetamide (Compound 107)

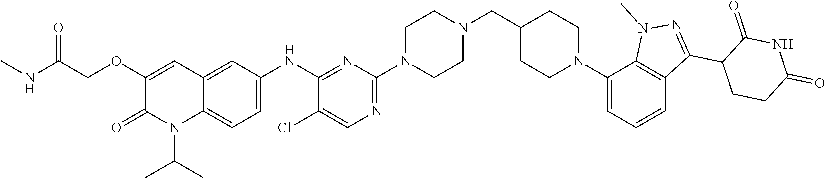 [SMILES: CNC(=O)COc1cc2cc(Nc3nc(N4CCN(CC5CCN(c6cccc7c(C8CCC(=O)NC8=O)nn(C)c67)CC5)CC4)ncc3Cl)ccc2n(C(C)C)c1=O]

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-isopropyl-2-oxo-1,2-dihydroquinolin-3-yl)oxy)-N-methylacetamide

To a stirred solution of 3-(1-methyl-7-(4-(piperazin-1-ylmethyl)piperidin-1-yl)-1H-indazol-3-yl)piperidine-2,6-dione hydrochloride (0.30 g, 0.65 mmol)(see, e.g., Example 1) and 2-((6-((2,5-dichloropyrimidin-4-yl)amino)-1-isopropyl-2-oxo-1,2-dihydroquinolin-3-yl)oxy)-N-methylacetamide (0.17 g, 0.39 mmol)(see, e.g., International Publication No. WO 2018/108704) in DMSO, N,N-diisopropylethylamine (0.67 g, 5.20 mmol) was added. The reaction mixture was stirred at 100° C. for 7 hours. The reaction mixture was poured into ice-cold water and stirred for 15 minutes. The resulting precipitate was filtered and dried to obtain crude product. The crude product was purified by prep-HPLC to afford 2-((6-((5-chloro-2-(4-((1-(3-(2,6-dioxopiperidin-3-yl)-1-methyl-1H-indazol-7-yl)piperidin-4-yl)methyl)piperazin-1-yl)pyrimidin-4-yl)amino)-1-isopropyl-2-oxo-1,2-dihydroquinolin-3-yl)oxy)-N-methylacetamide (0.125 mg) as an off-white solid.

LC-MS (ESI): m/z=822.76 [M−H]⁻.

¹H NMR (400 MHZ, DMSO-d₆): 10.88 (s, 1H), 8.22 (s, 1H), 8.06 (s, 1H), 7.96 (s, 2H), 7.69 (s, 2H), 7.37-7.35 (m, 1H), 7.04-7.00 (m, 3H), 4.55 (s, 2H), 4.34-4.31 (m, 1H), 3.79 (s, 3H), 3.66-3.64 (m, 4H), 3.24-3.22 (m, 2H), 2.67-2.54 (m, 7H), 2.51-2.50 (m, 1H), 2.48-2.42 (m, 4H), 2.33-2.31 (m, 2H), 2.19-2.16 (m, 1H), 1.90-1.74 (m, 4H), 1.57 (d, J=6.80 Hz, 6H), 1.40-1.37 (m, 2H).

### Example 33. Synthesis of 2-((6-((5-chloro-2-(4-((1-(3-(2,6-dioxopiperidin-3-yl)-1-methyl-1H-indazol-6-yl)piperidin-4-yl)methyl)piperazin-1-yl)pyrimidin-4-yl)amino)-1-isopropyl-2-oxo-1,2-dihydroquinolin-3-yl)oxy)-N-methylacetamide (Compound 108)

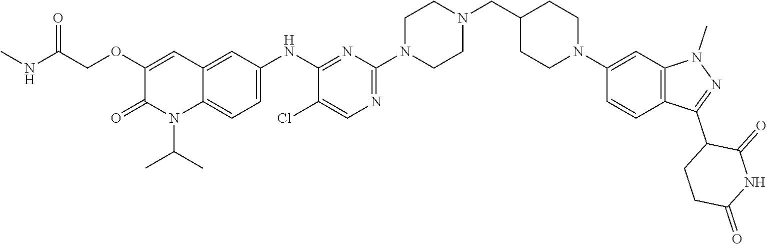 [SMILES: CNC(=O)COc1cc2cc(Nc3nc(N4CCN(CC5CCN(c6ccc7c(C8CCC(=O)NC8=O)nn(C)c7c6)CC5)CC4)ncc3Cl)ccc2n(C(C)C)c1=O]

### Preparation of 2-((6-((5-chloro-2-(4-((1-(3-(2,6-dioxopiperidin-3-yl)-1-methyl-1H-indazol-6-yl)piperidin-4-yl)methyl)piperazin-1-yl)pyrimidin-4-yl)amino)-1-isopropyl-2-oxo-1,2-dihydroquinolin-3-yl)oxy)-N-methylacetamide

To a stirred solution of 3-(1-methyl-6-(4-(piperazin-1-ylmethyl)piperidin-1-yl)-1H-indazol-3-yl)piperidine-2,6-dione hydrochloride (0.15 g, 0.32 mmol)(see, e.g., Example 2) and 2-((6-((2,5-dichloropyrimidin-4-yl)amino)-1-isopropyl-2-oxo-1,2-dihydroquinolin-3-yl)oxy)-N-methylacetamide (0.085 g, 0.19 mmol) in DMSO, N,N-diisopropylethylamine (0.33 g, 2.60 mmol) was added. The reaction mixture was stirred at 100° C. for 7 hours. The reaction mixture was poured into ice-cold water and stirred for 15 minutes. The resulting precipitate was filtered and dried to obtain crude product. The crude product was purified by prep-HPLC to afford 2-((6-((5-chloro-2-(4-((1-(3-(2,6-dioxopiperidin-3-yl)-1-methyl-1H-indazol-6-yl)piperidin-4-yl)methyl)piperazin-1-yl)pyrimidin-4-yl)amino)-1-isopropyl-2-oxo-1,2-dihydroquinolin-3-yl)oxy)-N-methylacetamide (19 mg) as an off-white solid.

LC-MS (ESI): m/z=822.61 [M−H]⁻.

¹H NMR (400 MHZ, DMSO-d₆): δ 10.84 (s, 1H), 8.84 (s, 1H), 8.06 (s, 1H), 7.96-7.94 (m, 2H), 7.70 (s, 2H), 7.47 (d, J=8.80 Hz, 1H), 7.03 (s, 1H), 6.91-6.82 (m, 2H), 5.56-5.49 (br s, 1H), 5.34 (br, 1H), 4.55 (s, 2H), 4.25 (q, J=5.20 Hz, 1H), 3.88 (s, 3H), 3.78 (d, J=11.60 Hz, 2H), 3.31 (br s, 4H), 2.73-2.59 (m, 7H), 2.40 (br s, 4H), 2.30-2.16 (m, 4H), 1.82-1.77 (m, 2H), 1.57 (d, J=6.80 Hz, 6H), 1.58-1.56 (m, 2H).

### Example 34. Synthesis of 2-((6-((5-chloro-2-(4-((2-(3-(2,6-dioxopiperidin-3-yl)-1-methyl-1H-indazol-6-yl)-2-azaspiro[3.5]nonan-7-yl)methyl)piperazin-1-yl)pyrimidin-4-yl)amino)-1-isopropyl-2-oxo-1,2-dihydroquinolin-3-yl)oxy)-N-methylacetamide (Compound 298)

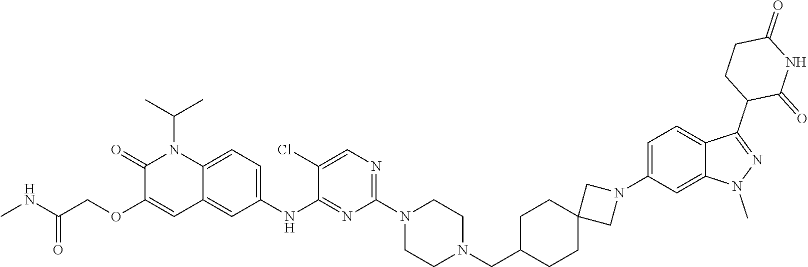 [SMILES: CNC(=O)COc1cc2cc(Nc3nc(N4CCN(CC5CCC6(CC5)CN(c5ccc7c(C8CCC(=O)NC8=O)nn(C)c7c5)C6)CC4)ncc3Cl)ccc2n(C(C)C)c1=O]

### Preparation of 2-((6-((5-chloro-2-(4-((2-(3-(2,6-dioxopiperidin-3-yl)-1-methyl-1H-indazol-6-yl)-2-azaspiro[3.5]nonan-7-yl)methyl)piperazin-1-yl)pyrimidin-4-yl)amino)-1-isopropyl-2-oxo-1,2-dihydroquinolin-3-yl)oxy)-N-methylacetamide

To a stirred solution of 3-(1-methyl-6-(7-(piperazin-1-ylmethyl)-2-azaspiro[3.5]nonan-2-yl)-1H-indazol-3-yl)piperidine-2,6-dione (0.14 g, 0.31 mmol)(see, e.g., Example 3) in DMSO (1.4 mL), 2-((6-((2,5-dichloropyrimidin-4-yl)amino)-1-isopropyl-2-oxo-1,2-dihydroquinolin-3-yl)oxy)-N-methylacetamide (0.067 g, 0.15 mmol) and DIPEA (0.27 mL, 1.53 mmol) was added. The reaction was heated to 100° C. for 2 hours. The reaction mixture was poured into ice-cold water. The resulting solid was filtered and dried to obtain crude product. The crude product was purified by prep-HPLC to afford 2-((6-((5-chloro-2-(4-((2-(3-(2,6-dioxopiperidin-3-yl)-1-methyl-1H-indazol-6-yl)-2-azaspiro[3.5]nonan-7-yl)methyl)piperazin-1-yl)pyrimidin-4-yl)amino)-1-isopropyl-2-oxo-1,2-dihydroquinolin-3-yl)oxy)-N-methylacetamide (0.023 g) as an off-white solid.

LC-MS (ESI): m/z=864.54 [M+H]⁺.

¹H NMR (400 MHZ, DMSO-d₆): δ 10.83 (s, 1H), 8.84 (s, 1H), 8.05 (s, 1H), 7.96 (s, 2H), 7.69 (s, 1H), 7.46 (d, J=8.40 Hz, 1H), 7.03 (s, 1H), 7.35 (d, J=8.80 Hz, 1H), 6.30 (s, 1H), 5.32-5.26 (m, 1H), 4.55 (s, 2H), 4.25-4.21 (m, 1H), 3.83 (s, 3H), 3.64-3.55 (m, 8H), 2.67 (d, J=4.80 Hz, 3H), 2.63-2.59 (m, 3H), 2.37-2.25 (m, 5H), 2.17-2.11 (m, 3H), 1.91-1.86 (m, 3H), 1.75-1.72 (m, 2H), 1.57 (d, J=6.80 Hz, 8H), 1.01-0.99 (m, 2H).

### Example 35. Synthesis of 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-isopropyl-2-oxo-1,2-dihydroquinolin-3-yl)oxy)-N-methylacetamide (Compound 311)

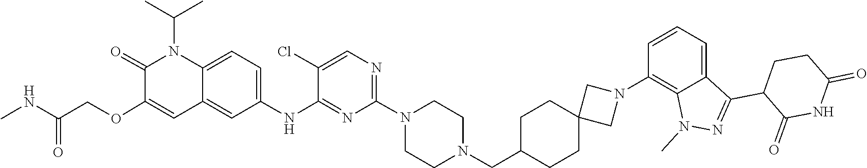 [SMILES: CNC(=O)COc1cc2cc(Nc3nc(N4CCN(CC5CCC6(CC5)CN(c5cccc7c(C8CCC(=O)NC8=O)nn(C)c57)C6)CC4)ncc3Cl)ccc2n(C(C)C)c1=O]

### Preparation of 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-isopropyl-2-oxo-1,2-dihydroquinolin-3-yl)oxy)-N-methylacetamide

To a stirred solution of 3-(1-methyl-7-(7-(piperazin-1-ylmethyl)-2-azaspiro[3.5]nonan-2-yl)-1H-indazol-3-yl)piperidine-2,6-dione (0.18 g, 0.38 mmol)(see, e.g., Example 4) and 2-((6-((2,5-dichloropyrimidin-4-yl)amino)-1-isopropyl-2-oxo-1,2-dihydroquinolin-3-yl)oxy)-N-methylacetamide (0.10 g, 0.23 mmol) in DMSO (2.0 mL), N,N-diisopropylethylamine (0.53 mL, 3.1 mmol) was added. The reaction mixture was stirred at 100° C. for 16 hours. The reaction mixture was cooled to room temperature and poured into ice water. After stirring for 15 minutes, the resulting precipitate was filtered and dried to obtain crude product. The crude product was purified by preparative HPLC to afford 2-((6-((5-chloro-2-(4-((2-(3-(2,6-dioxopiperidin-3-yl)-1-methyl-1H-indazol-7-yl)-2-azaspiro[3.5]nonan-7-yl)methyl)piperazin-1-yl)pyrimidin-4-yl)amino)-1-isopropyl-2-oxo-1,2-dihydroquinolin-3-yl)oxy)-N-methylacetamide (0.025 g) as an off white solid.

LC-MS (ESI): m/z=862.67 [M−H]⁻.

¹H NMR (400 MHZ, DMSO-d₆): δ 10.85 (s, 1H), 8.84 (s, 1H), 8.05 (s, 1H), 7.96 (s, 2H), 7.69 (s, 2H), 7.20 (d, J=8.00 Hz, 1H), 7.03 (s, 1H), 6.98 (t, J=7.60 Hz, 1H), 6.72 (d, J=7.20 Hz, 1H), 5.35 (br, 1H), 4.55 (s, 2H), 4.32-4.29 (m, 1H), 4.17 (s, 3H), 3.64-3.54 (m, 8H), 2.68-2.56 (m, 5H), 2.37-2.30 (m, 5H), 2.18-2.12 (m, 3H), 1.98-1.90 (m, 2H), 1.73 (d, J=11.60 Hz, 2H), 1.58-1.48 (m, 9H), 1.00-0.97 (m, 2H).

### Example 36. Synthesis of 2-((6-((5-chloro-2-((((1s,4s)-4-((4-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (Compound 277b)

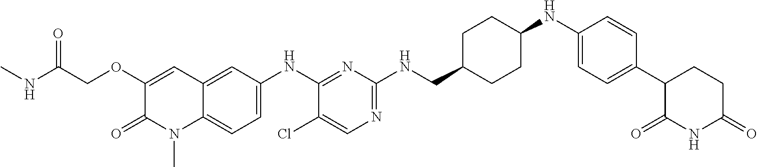 [SMILES: CNC(=O)COc1cc2cc(Nc3nc(NC[C@H]4CC[C@@H](Nc5ccc(C6CCC(=O)NC6=O)cc5)CC4)ncc3Cl)ccc2n(C)c1=O]

### Preparation of tert-butyl (((1s,4s)-4-((4-(2,6-bis(benzyloxy)pyridin-3-yl)phenyl)amino)cyclohexyl)methyl) carbamate

To a stirred solution of 2,6-bis(benzyloxy)-3-(4-bromophenyl)pyridine (0.76 g, 1.53 mmol) in 1,4-dioxane (14 mL), tert-butyl (((1s,4s)-4-aminocyclohexyl)methyl) carbamate (0.50 g, 2.19 mmol) was added. The reaction was purged with argon for 15 minutes. Potassium tert-butoxide (0.49 g, 4.38 mmol) and Pd (t-Bu₃P)₂ (0.56 g, 0.10 mmol) were added. The heated reaction mixture at 100° C. for 3 hours. The reaction was quenched with water and extracted into ethyl acetate. The combined organic layers were washed with brine solution, dried over anhydrous Na₂SO₄, filtered, and concentrated under vacuum to obtain crude product. The crude was purified by flash column chromatography (SiO₂, 230-400, 15% ethyl acetate in petroleum ether) to afford tert-butyl (((1s,4s)-4-((4-(2,6-bis(benzyloxy)pyridin-3-yl)phenyl)amino)cyclohexyl)methyl) carbamate (0.480 g) as a pale brown semi-solid.

LC-MS (ESI): m/z=594.65 [M+H]⁺.

### Preparation of tert-butyl (((1s,4s)-4-((4-(2,6-dioxopiperidin-3-yl)phenyl)amino)cyclohexyl)methyl) carbamate

To a stirred solution of tert-butyl (((1s,4s)-4-((4-(2,6-bis(benzyloxy)pyridin-3-yl)phenyl)amino)cyclohexyl)methyl) carbamate (0.45 g, 0.76 mmol) in THF (9 mL), 20% Pd(OH)₂ on carbon, moisture 50% wet (0.63 g, 6.07 mmol) was added. The reaction was put under hydrogen atmosphere (balloon pressure) and stirred at room temperature for 4 hours. The reaction mixture was filtered through a pad of celite. The celite was washed with THE, and the filtrate was concentrated under vacuum to obtain crude product. The crude product was triturated with n-pentane to afford tert-butyl (((1s,4s)-4-((4-(2,6-dioxopiperidin-3-yl)phenyl)amino)cyclohexyl)methyl) carbamate (0.35 g) as an off-white solid.

LC-MS (ESI): m/z=416.48 [M+H]⁺.

### Preparation of 3-(4-(((1s,4s)-4-(aminomethyl)cyclohexyl)amino)phenyl)piperidine-2,6-dione

To a stirred solution of tert-butyl (((1s,4s)-4-((4-(2,6-dioxopiperidin-3-yl)phenyl)amino)cyclohexyl)methyl) carbamate (0.30 g, 0.72 mmol) in DCM (3.0 mL), 4 M HCl in dioxane (3.0 mL) was added, and the reaction was stirred at room temperature for 4 hours. The solvent was evaporated under vacuum to obtain crude product. The crude product was triturated with n-pentane to afford 3-(4-(((1s,4s)-4-(aminomethyl)cyclohexyl)amino)phenyl)piperidine-2,6-dione (0.21 g) as an off-white solid.

LC-MS (ESI): m/z=316.45 [M+H]⁺.

### Preparation of 2-((6-((5-chloro-2-((((1s,4s)-4-((4-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide

To a stirred solution of 3-(4-(((1s,4s)-4-(aminomethyl)cyclohexyl)amino)phenyl)piperidine-2,6-dione.HCl (0.69 g, 0.69 mmol) in DMSO (6.6 mL), 2-((6-((2,5-dichloropyrimidin-4-yl)amino)-1-methyl-2-oxo-1,2-dihydroquinolin-3-yl)oxy)-N-methylacetamide (0.14 g, 0.34 mmol) and DIPEA (1.2 mL, 5.5 mmol) were added. The reaction mixture was heated at 100° C. for 6 hours. The reaction was quenched with ice-cold water. The resulting precipitate was filtered, washed with water, and dried under vacuum to obtain crude product. The crude was purified by preparative HPLC to afford 2-((6-((5-chloro-2-((((1s,4s)-4-((4-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (0.017 g) as a light brown solid.

LC-MS (ESI): m/z=687.48 [M+H]⁺.

¹H NMR (400 MHZ, DMSO-d₆): δ 10.72 (s, 1H), 8.66 (d, J=17.8 Hz, 1H), 8.38 (bs, 1H), 8.2-7.7 (m, 3H), 7.44 (d, J=9.1 Hz, 1H), 7.20 (bs, J=12.0 Hz, 1H), 6.87 (d, J=8.3 Hz, 1H), 6.53 (s, 1H), 5.33 (d, 1H), 4.55 (s, 2H), 3.67 (s, 3H), 3.14 (m, 3H), 2.66 (d, 1H), 2.60 (s, 1H), 2.03 (m, 2H), 1.52 (m, 9H).

### Example 37. Synthesis of 2-((6-((5-chloro-2-(4-(1-(3-(2,6-dioxopiperidin-3-yl)-1-methyl-1H-indazol-6-yl) azetidin-3-yl)piperidin-1-yl)pyrimidin-4-yl)amino)-1-methyl-2-oxo-1,2-dihydroquinolin-3-yl)oxy)-N-methylacetamide (Compound 292)

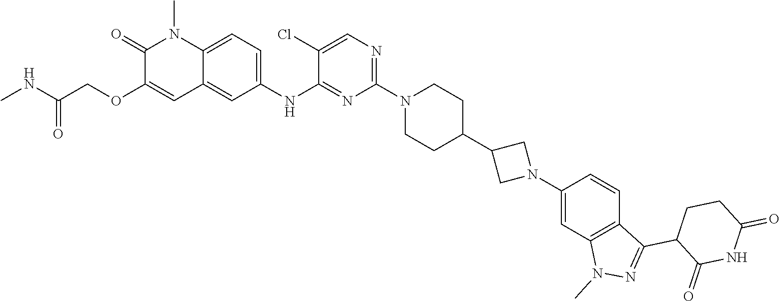 [SMILES: CNC(=O)COc1cc2cc(Nc3nc(N4CCC(C5CN(c6ccc7c(C8CCC(=O)NC8=O)nn(C)c7c6)C5)CC4)ncc3Cl)ccc2n(C)c1=O]

### Preparation of tert-butyl 4-(1-(3-(2,6-bis(benzyloxy)pyridin-3-yl)-1-methyl-1H-indazol-6-yl) azetidin-3-yl)piperidine-1-carboxylate

A solution of tert-butyl 4-(azetidin-3-yl)piperidine-1-carboxylate (0.30 g, 1.24 mmol), 3-(2,6-bis(benzyloxy)pyridin-3-yl)-6-bromo-1-methyl-1H-indazole (0.62 g, 1.24 mmol), and potassium tertiary butoxide (0.42 g, 3.74 mmol) in 1,4-dioxane (6.0 mL) was degassed with argon for 5 minutes. Bis(tri-tert-butylphosphine)palladium (0) (0.06 g, 0.12 mmol) was added to the reaction, and the reaction was degassed for 5 minutes. The reaction mixture was heated at 100° C. for 4 hours. The reaction mixture was filtered through a pad of celite and concentrated to obtain crude product. The crude product was purified by column chromatography (SiO₂, 230-400 mesh, 40% ethyl acetate in petroleum ether) to afford tert-butyl 4-(1-(3-(2,6-bis(benzyloxy)pyridin-3-yl)-1-methyl-1H-indazol-6-yl) azetidin-3-yl)piperidine-1-carboxylate (0.55 g) as a brown solid.

LC-MS (ESI): m/z=660.68 [M+H]⁺.

### Preparation of tert-butyl 4-(1-(3-(2,6-dioxopiperidin-3-yl)-1-methyl-1H-indazol-6-yl) azetidin-3-yl)piperidine-1-carboxylate

A solution of tert-butyl 4-(1-(3-(2,6-bis(benzyloxy)pyridin-3-yl)-1-methyl-1H-indazol-6-yl) azetidin-3-yl)piperidine-1-carboxylate (0.45 g, 0.68 mmol) in THF (4.5 mL) was degassed with nitrogen. 20% palladium hydroxide (0.90 g) was added. The reaction was put under hydrogen atmosphere (20 psi) and stirred at room temperature for 8 hours. The reaction mixture was filtered through a pad of celite. The celite was washed with THF, and the filtrate was concentrated to obtain crude product. The crude product was washed with n-pentane and dried to afford tert-butyl 4-(1-(3-(2,6-dioxopiperidin-3-yl)-1-methyl-1H-indazol-6-yl) 258azetidine-3-yl)piperidine-1-carboxylate (0.30 g) as a brown gum.

LC-MS (ESI): m/z=486.59 [M+H]⁺.

### Preparation of 3-(1-methyl-6-(3-(piperidin-4-yl) azetidin-1-yl)-1H-indazol-3-yl)piperidine-2,6-dione

A stirred solution of tert-butyl 4-(1-(3-(2,6-dioxopiperidin-3-yl)-1-methyl-1H-indazol-6-yl) azetidin-3-yl)piperidine-1-carboxylate in DCM (6 mL) was cooled to 0° C. TFA (0.30 mL) was added, and the reaction was allowed to warm to room temperature over 1 hour with stirring. The reaction mixture was concentrated to afford 3-(1-methyl-6-(3-(piperidin-4-yl) 258azetidine-1-yl)-1H-indazol-3-yl)piperidine-2,6-dione (0.20 g).

LC-MS (ESI): m/z=382.47 [M+H]⁺.

### Preparation of 2-((6-((5-chloro-2-(4-(1-(3-(2,6-dioxopiperidin-3-yl)-1-methyl-1H-indazol-6-yl) azetidin-3-yl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(3-(piperidin-4-yl) azetidin-1-yl)-1H-indazol-3-yl)piperidine-2,6-dione (0.20 g, 0.53 mmol) and 2-((6-((2,5-dichloropyrimidin-4-yl)amino)-1-methyl-2-oxo-1,2-dihydroquinolin-3-yl)oxy)-N-methylacetamide (0.10 g, 0.26 mmol) in DMSO, N,N-diisopropylethylamine (0.9 mL, 5.25 mmol) was added. The reaction mixture was stirred at 100° C. for 4 hours. The reaction was poured into ice-cold water and stirred for 15 minutes. The resulting precipitate was filtered and dried to obtain crude product. The crude product was purified by prep-HPLC to afford 2-((6-((5-chloro-2-(4-(1-(3-(2,6-dioxopiperidin-3-yl)-1-methyl-1H-indazol-6-yl) azetidin-3-yl)piperidin-1-yl)pyrimidin-4-yl)amino)-1-methyl-2-oxo-1,2-dihydroquinolin-3-yl)oxy)-N-methylacetamide (25 mg) as a brown solid.

LC-MS (ESI): m/z=753.56 [M+H]⁺.

¹H NMR (400 MHZ, DMSO-d₆): δ 10.90 (s, 1H), 8.84 (s, 1H), 8.05 (s, 1H), 7.96-7.93 (m, 2H), 7.75 (dd, J=2.00, 9.20 Hz, 1H), 7.47 (d, J=10.00 Hz, 2H), 7.10 (s, 1H), 6.36-6.32 (m, 2H), 4.58-4.50 (m, 4H), 4.25-4.21 (m, 1H), 3.97 (t, J=7.60 Hz, 2H), 3.84 (s, 3H), 3.68 (s, 3H), 3.60 (t, J=6.40 Hz, 2H), 2.84 (t, J=12.00 Hz, 2H), 2.68 (d, J=4.80 Hz, 3H), 2.60-2.59 (m, 3H), 2.33-2.27 (m, 1H), 2.17-2.14 (m, 1H), 1.74-1.71 (d, J=10.00 Hz, 3H), 1.24 (s, 2H), 1.07-1.04 (m, 2H).

### Example 38. Synthesis of 2-((6-((5-chloro-2-((((1s,4s)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide (Compound 293a)

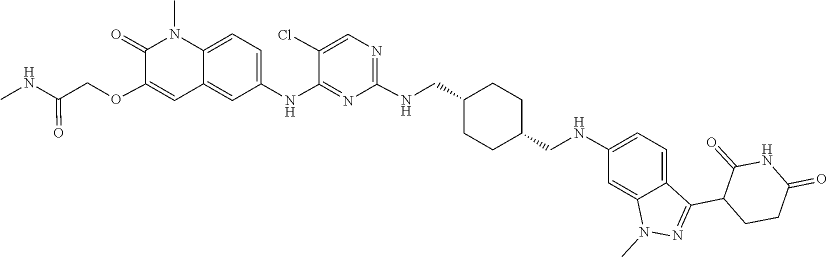 [SMILES: CNC(=O)COc1cc2cc(Nc3nc(NC[C@H]4CC[C@@H](CNc5ccc6c(C7CCC(=O)NC7=O)nn(C)c6c5)CC4)ncc3Cl)ccc2n(C)c1=O]

### Preparation of tert-butyl (((1s,4s)-4-(((3-(2,6-bis(benzyloxy)pyridin-3-yl)-1-methyl-1H-indazol-6-yl)amino)methyl)cyclohexyl)methyl) carbamate

A solution of tert-butyl (((1s,4s)-4-(aminomethyl)cyclohexyl)methyl) carbamate (0.2 g, 0.82 mmol, 1 eq.), 3-(2,6-bis(benzyloxy)pyridin-3-yl)-6-bromo-1-methyl-1H-indazole (0.3 g, 0.57 mmol, 1 eq.), and potassium tert-butoxide (0.13 g, 1.24 mmol) in 1,4-dioxane (4 mL) was degassed with nitrogen for 15 minutes. Bis(tri-tert-butylphosphine)palladium (0) (0.02 g, 1.23 mmol, 0.05 eq.) was added, and the resulting mixture was degassed again for 10 minutes. The reaction was heated with stirring at 80° C. for 24 hours. The reaction mixture was filtered through a pad of celite and concentrated under reduced pressure to obtain crude product. The crude product was purified by flash column (SiO₂, mesh size: 100-200, 25% EtOAc in petroleum ether) to afford tert-butyl (((1s,4s)-4-(((3-(2,6-bis(benzyloxy)pyridin-3-yl)-1-methyl-1H-indazol-6-yl)amino)methyl)cyclohexyl)methyl) carbamate (0.40 g).

LC-MS (ESI): m/z=662.74 [M+H]⁺.

### Preparation of tert-butyl (((1s,4s)-4-(((3-(2,6-dioxopiperidin-3-yl)-1-methyl-1H-indazol-6-yl)amino)methyl)cyclohexyl)methyl) carbamate

To stirred solution of tert-butyl (((1s,4s)-4-(((3-(2,6-bis(benzyloxy)pyridin-3-yl)-1-methyl-1H-indazol-6-yl)amino)methyl)cyclohexyl)methyl) carbamate (0.40 g, 0.60 mmol) in ethyl acetate (24 mL), palladium on activated carbon (0.38 g) was added. The reaction was put under hydrogen atmosphere (balloon pressure) and stirred at room temperature for 3 hours. The reaction mixture was filtered through a pad of celite, and the filtrate concentrated under reduced pressure to obtain crude product. The crude product was purified by flash column (SiO₂, mesh size 100-200, 60% EtOAc in petroleum ether) to afford tert-butyl (((1s,4s)-4-(((3-(2,6-dioxopiperidin-3-yl)-1-methyl-1H-indazol-6-yl)amino)methyl)cyclohexyl)methyl) carbamate (0.08 g).

LC-MS (ESI): m/z=484.56 [M+H]⁺.

### Preparation of 3-(6-((((1s,4s)-4-(aminomethyl)cyclohexyl)methyl)amino)-1-methyl-1H-indazol-3-yl)piperidine-2,6-dione

To a stirred suspension of tert-butyl (((1s,4s)-4-(((3-(2,6-dioxopiperidin-3-yl)-1-methyl-1H-indazol-6-yl)amino)methyl)cyclohexyl)methyl) carbamate (0.08 g, 0.16 mmol) in DCM (1.6 mL), hydrochloric acid was added (0.8 mL) at 0° C. The reaction mixture was allowed to warm to room temperature over 3 hours with stirring. The reaction mixture was concentrated under reduced pressure to obtain 3-(6-((((1s,4s)-4-(aminomethyl)cyclohexyl)methyl)amino)-1-methyl-1H-indazol-3-yl)piperidine-2,6-dione (0.04 g).

LC-MS (ESI): m/z=384.45 [M+H]⁺.

### Preparation of 2-((6-((5-chloro-2-((((1s,4s)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide

To a stirred solution of 3-(6-((((1s,4s)-4-(aminomethyl)cyclohexyl)methyl)amino)-1-methyl-1H-indazol-3-yl)piperidine-2,6-dione (0.04 g, 0.10 mmol) and 2-((6-((2,5-dichloropyrimidin-4-yl)amino)-1-methyl-2-oxo-1,2-dihydroquinolin-3-yl)oxy)-N-methylacetamide (0.03 g, 0.07 mmol) in dimethyl sulfoxide (0.8 mL), N,N-diisopropylethylamine (0.09 mL, 0.52 mmol) and p-TSA were at room temperature. The resulting mixture was stirred at 100° C. for 3 hours. The reaction was poured into water, and the resulting precipitate was filtered to obtain crude product. The crude product was purified via prep-HPLC to afford 2-((6-((5-chloro-2-((((1s,4s)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide (8 mg).

LC-MS (ESI): m/z=753.56 [M−H]⁻.

¹H NMR (400 MHZ, CDCl₃): δ 8.68 (s, 1H), 7.82-7.84 (m, 3H), 7.43 (d, J=8.80 Hz, 1H), 6.89-6.94 (m, 3H), 6.52 (d, J=8.40 Hz, 1H), 6.25 (s, 1H), 5.80 (s, 1H), 4.55 (s, 2H), 4.15-4.17 (m, 1H), 3.79 (s, 3H), 3.64 (s, 3H), 3.19 (s, 2H), 2.93 (d, J=51.20 Hz, 2H), 2.67 (d, J=4.80 Hz, 3H), 2.60 (t, J=6.40 Hz, 2H), 2.20-2.22 (m, 1H), 2.12-2.13 (m, 1H), 1.75 (s, 3H), 1.36 (t, J=8.00 Hz, 8H), 1.24 (s, 1H).

### Example 39. Synthesis of 2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3S)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide] (Compound 294a)

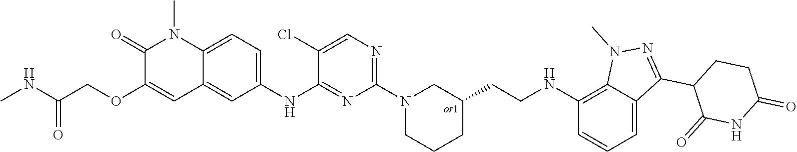 [SMILES: CNC(=O)COc1cc2cc(Nc3nc(N4CCC[C@@H](CCNc5cccc6c(C7CCC(=O)NC7=O)nn(C)c56)C4)ncc3Cl)ccc2n(C)c1=O]

### Preparation of tert-butyl 3-(2-((3-(2,6-bis(benzyloxy)pyridin-3-yl)-1-methyl-1H-indazol-7-yl)amino)ethyl)piperidine-1-carboxylate (Isomer 1 and 2

A stirred solution of 3-(2,6-bis(benzyloxy)pyridin-3-yl)-7-bromo-1-methyl-1H-indazole (1.0 g, 1.99 mmol) and tert-butyl 3-(2-aminoethyl)piperidine-1-carboxylate (0.45 g, 1.99 mmol) in 1,4-dioxane (15 mL) was purged with argon. Sodium tertbutoxide (0.576 g, 5.99 mmol) and Brettphos palladium G3 (0.091 g, 0.10 mmol) were added, and the reaction was stirred at 100° C. for 6 hours. The reaction mixture was diluted with ethyl acetate and filtered through a pad of celite. The celite was washed with 10% MeOH in DCM, and the filtrate was concentrated under reduced pressure to obtain crude product. The crude product was purified by flash column chromatography (SiO₂; 17% ethyl acetate in petroleum ether) to afford tert-butyl 3-(2-((3-(2,6-bis(benzyloxy)pyridin-3-yl)-1-methyl-1H-indazol-7-yl)amino)ethyl)piperidine-1-carboxylate (0.92 g) as pale green semi-solid.

LC-MS (ESI): m/z=648.67 [M+H]⁺.

The enantiomers were separated via SFC(Column: Chiralpak IJ (4.6×250 mm, internal diameter: 5 μm); mobile phase: Phase A (CO₂), and Phase B (MeCN/MeOH with 0.2% NH₃·H₂O); isocratic elution B %: 50%; flow rate: 3.0 mL/min; detector: PDA; wavelength: 315 nm; column temperature: 30° C.; back pressure: 100 bar) to afford tert-butyl 3-(2-((3-(2,6-bis(benzyloxy)pyridin-3-yl)-1-methyl-1H-indazol-7-yl)amino)ethyl)piperidine-1-carboxylate (Isomer-1, Rt=1.93 min)(0.42 g) and tert-butyl 3-(2-((3-(2,6-bis(benzyloxy)pyridin-3-yl)-1-methyl-1H-indazol-7-yl)amino)ethyl)piperidine-1-carboxylate (Isomer-2, Rt=3.31 min)(0.36 g).
Isomer 1: LC-MS (ESI): m/z=648.73 [M+H]⁺
Isomer 2: LC-MS (ESI): m/z=648.65 [M+H]⁺.

### Preparation of tert-butyl 3-(2-((3-(2,6-dioxopiperidin-3-yl)-1-methyl-1H-indazol-7-yl)amino)ethyl)piperidine-1-carboxylate (Isomer 2)

To a stirred solution of tert-butyl 3-(2-((3-(2,6-bis(benzyloxy)pyridin-3-yl)-1-methyl-1H-indazol-7-yl)amino)ethyl)piperidine-1-carboxylate (Isomer 2) (0.35 g, 0.54 mmol) in THF (14 mL), 20% Pd(OH)₂ on carbon, 100% (w/w)(0.39 g, 2.71 mmol) was added. The reaction was put under hydrogen atmosphere (15 psi) and stirred for 18 hours at room temperature. The reaction mixture was diluted with THF and filtered through a pad of celite. The filtrate was concentrated to afford tert-butyl 3-(2-((3-(2,6-dioxopiperidin-3-yl)-1-methyl-1H-indazol-7-yl)amino)ethyl)piperidine-1-carboxylate (Isomer 2) (0.19 g) as a pale brown semi-solid.

LC-MS (ESI): m/z=492.5 [M+Na]⁺.

### Preparation of 3-(1-methyl-7-((2-(piperidin-3-yl)ethyl)amino)-1H-indazol-3-yl)piperidine-2,6-dione (Isomer 2)

To a stirred solution of tert-butyl 3-(2-((3-(2,6-dioxopiperidin-3-yl)-1-methyl-1H-indazol-7-yl)amino)ethyl)piperidine-1-carboxylate (Isomer 2) (0.18 g, 0.383 mmol) in DCM (3.6 mL), TFA (1.8 mL) was added at 0° C. under a nitrogen atmosphere. The reaction was allowed to warm to room temperature over 3 hours with stirring. The reaction mixture was concentrated under vacuum to afford 3-(1-methyl-7-((2-(piperidin-3-yl)ethyl)amino)-1H-indazol-3-yl)piperidine-2,6-dione (Isomer 2) (0.15 g) as a pale brown semi-solid.

LC-MS (ESI): m/z=370.42 [M+H]⁺.

### Preparation of 2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3S)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide]

To a stirred solution of 2-((6-((2,5-dichloropyrimidin-4-yl)amino)-1-methyl-2-oxo-1,2-dihydroquinolin-3-yl)oxy)-N-methylacetamide (0.082 g, 0.20 mmol) in DMSO (3.0 mL), 3-(1-methyl-7-((2-(piperidin-3-yl)ethyl)amino)-1H-indazol-3-yl)piperidine-2,6-dione (Isomer 2) (0.15 g, 0.40 mmol) and DIPEA (0.5 mL, 3.20 mmol) were added. The reaction was heated to 100° C. for 6 hours. The reaction mixture was quenched with ice-cold water. The resulting precipitate was filtered and dried to obtain crude product. The crude product was purified by prep-HPLC to afford 2-((6-((5-chloro-2-((3S)-3-(2-((3-(2,6-dioxopiperidin-3-yl)-1-methyl-1H-indazol-6-yl)amino)ethyl)piperidin-1-yl)pyrimidin-4-yl)amino)-1-methyl-2-oxo-1,2-dihydroquinolin-3-yl)oxy)-N-methylacetamide [rel-2-((6-((5-chloro-2-((3S)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide] (29 mg) as an off-white solid.

LC-MS (ESI): m/z=741.49 [M+H]⁺.

¹H NMR (400 MHZ, DMSO-d₆): δ 10.85 (s, 1H), 8.82 (s, 1H), 8.03 (s, 1H), 7.91-7.89 (m, 2H), 7.76 (d, J=7.60 Hz, 1H), 7.12 (s, 1H), 6.92 (d, J=8.00 Hz, 1H), 5.23 (s, 1H), 4.54 (s, 2H), 4.44 (d, J=11.20 Hz, 1H), 4.26-4.23 (m, 2H), 4.18 (s, 3H), 3.54 (s, 3H), 3.07-2.95 (m, 3H), 2.78-2.68 (m, 2H), 2.66-2.58 (m, 5H), 2.29-2.26 (m, 2H), 2.17-2.15 (m, 1H), 1.96-1.86 (m, 2H), 1.76-1.62 (m, 4H), 1.49-1.41 (m, 2H).

### Example 40. Synthesis of 2-((6-((5-chloro-2-((3R)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3R)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide] (Compound 294b)

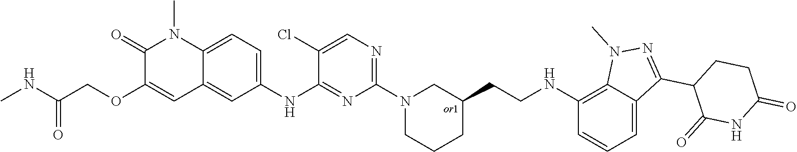 [SMILES: CNC(=O)COc1cc2cc(Nc3nc(N4CCC[C@H](CCNc5cccc6c(C7CCC(=O)NC7=O)nn(C)c56)C4)ncc3Cl)ccc2n(C)c1=O]

### Preparation of tert-butyl 3-(2-((3-(2,6-dioxopiperidin-3-yl)-1-methyl-1H-indazol-7-yl)amino)ethyl)piperidine-1-carboxylate (Isomer 1)

To a stirred solution of tert-butyl 3-(2-((3-(2,6-bis(benzyloxy)pyridin-3-yl)-1-methyl-1H-indazol-7-yl)amino)ethyl)piperidine-1-carboxylate (Isomer 1) (0.21 g, 0.32 mmol) in THF (4 mL), AcOH (0.2 mL), DMF (0.4 mL), and 20% Pd(OH)₂ on carbon 200% (w/w)(0.44 g) were added. The reaction was put under hydrogen atmosphere (15 psi) and stirred at room temperature for 18 hours. The reaction mixture was diluted with THF and filtered through a pad of celite. The filtrate was concentrated to afford tert-butyl 3-(2-((3-(2,6-dioxopiperidin-3-yl)-1-methyl-1H-indazol-7-yl)amino)ethyl)piperidine-1-carboxylate (Isomer 1) (0.14 g) as a pale brown semi-solid.

LC-MS (ESI): m/z=370.42 [M+H]⁺.

### Preparation of 3-(1-methyl-7-((2-(piperidin-3-yl)ethyl)amino)-1H-indazol-3-yl)piperidine-2,6-dione (Isomer 1)

To a stirred solution of tert-butyl 3-(2-((3-(2,6-dioxopiperidin-3-yl)-1-methyl-1H-indazol-7-yl)amino)ethyl)piperidine-1-carboxylate (Isomer 1) (0.16 g, 0.34 mmol) in DCM (0.8 mL), TFA (0.64 mL) was added at 0° C. under nitrogen atmosphere. The reaction was allowed to warm to room temperature over 3 hours with stirring. The reaction mixture was concentrated under vacuum to afford 3-(1-methyl-7-((2-(piperidin-3-yl)ethyl)amino)-1H-indazol-3-yl)piperidine-2,6-dione (Isomer 1) (0.19 g) as a pale brown semi-solid.

LC-MS (ESI): m/z=370.42 [M+H]⁺.

### Preparation of 2-((6-((5-chloro-2-((3R)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide

[rel-2-((6-((5-chloro-2-((3R)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide]

To a stirred solution of 3-(1-methyl-7-((2-(piperidin-3-yl)ethyl)amino)-1H-indazol-3-yl)piperidine-2,6-dione (Isomer 1) (0.19 g, 0.51 mmol) in DMSO (3.8 mL), DIPEA (0.7 mL, 4.11 mmol) and 2-((6-((2,5-dichloropyrimidin-4-yl)amino)-1-methyl-2-oxo-1,2-dihydroquinolin-3-yl)oxy)-N-methylacetamide (0.11 g, 0.26 mmol) were add. The reaction was heated to 100° C. for 6 hours. The reaction mixture was quenched with ice-cold water. The resulting precipitate was filtered and dried to get obtain crude product. The crude product was purified by prep-HPLC to afford 2-((6-((5-chloro-2-((3R)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide [rel-2-((6-((5-chloro-2-((3R)-3-(2-((3-(2,6-dioxopiperidin-3-yl)-1-methyl-1H-indazol-7-yl)amino)ethyl)piperidin-1-yl)pyrimidin-4-yl)amino)-1-methyl-2-oxo-1,2-dihydroquinolin-3-yl)oxy)-N-methylacetamide] (0.018 g) as an off-white solid.

LC-MS (ESI): m/z=741.57 [M+H]⁺.

¹H NMR (400 MHZ, DMSO-d₆): δ 10.85 (s, 1H), 8.82 (s, 1H), 8.03 (s, 1H), 7.92 (d, J=4.40 Hz, 1H), 7.89 (s, 1H), 7.76 (d, J=8.80 Hz, 1H), 7.37 (d, J=7.60 Hz, 1H), 7.12 (s, 1H), 6.91 (d, J=8.00 Hz, 1H), 6.77-6.64 (m, 1H), 6.29-6.28 (m, 1H), 5.35 (s, 1H), 4.54 (s, 2H), 4.44 (d, J=11.60 Hz, 1H), 4.26-4.18 (m, 4H), 3.54 (s, 3H), 3.07-3.01 (m, 3H), 2.95-2.78 (m, 1H), 2.61-2.66 (m, 4H), 2.29-2.25 (m, 1H), 2.18-2.13 (m, 1H), 1.89-1.86 (m, 1H), 1.69-1.62 (m, 4H), 1.36-1.24 (m, 4H).

### Example 41. Synthesis of 2-((6-((5-chloro-2-((((3S)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide (Compound 295b)

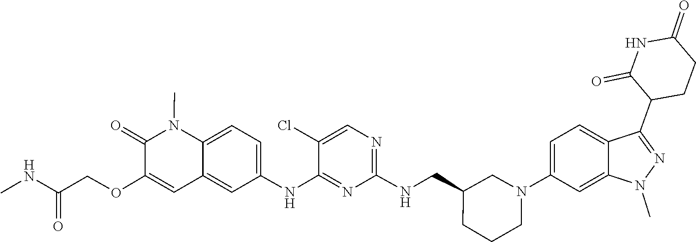 [SMILES: CNC(=O)COc1cc2cc(Nc3nc(NC[C@@H]4CCCN(c5ccc6c(C7CCC(=O)NC7=O)nn(C)c6c5)C4)ncc3Cl)ccc2n(C)c1=O]

### Preparation of tert-butyl(S)-((1-(3-(2,6-bis(benzyloxy)pyridin-3-yl)-1-methyl-1H-indazol-6-yl)piperidin-3-yl)methyl) carbamate

To a stirred suspension of 3-(2,6-bis(benzyloxy)pyridin-3-yl)-6-bromo-1-methyl-1H-indazole (0.5 g, 1.0 mmol) in 1,4-dioxane, tert-butyl (R)-(piperidin-3-ylmethyl) carbamate (0.32 g, 1.5 mmol) and cesium carbonate (0.98 g, 3.0 mmol) were added. The reaction was purged with argon for 15 minutes. RuPhos (0.093 g, 0.2 mmol) and RuPhos-Pd-G3 (0.042 g, 0.05 mmol) were added to the reaction, and the reaction was heated at 100° C. for 6 hours. The reaction mixture was filtered through a pad of celite and concentrated to obtain crude product. The crude product was purified by column chromatography (SiO₂, 100-200 mesh, 1-2% methanol in DCM) to afford tert-butyl(S)-((1-(3-(2,6-bis(benzyloxy)pyridin-3-yl)-1-methyl-1H-indazol-6-yl)piperidin-3-yl)methyl) carbamate (0.45 g) as an off-white solid.

LC-MS (ESI): m/z=634.75 [M+H]⁺.

### Preparation of tert-butyl (((3S)-1-(3-(2,6-dioxopiperidin-3-yl)-1-methyl-1H-indazol-6-yl)piperidin-3-yl)methyl) carbamate

A stirred solution of tert-butyl(S)-((1-(3-(2,6-bis(benzyloxy)pyridin-3-yl)-1-methyl-1H-indazol-6-yl)piperidin-3-yl)methyl) carbamate (0.35 g, 0.56 mmol) in THF (19.0 mL) was degassed with nitrogen. 20% palladium hydroxide (0.90 g) was added to the reaction. The reaction was put under hydrogen atmosphere (15 psi) and stirred at room temperature for 8 hours. The reaction mixture was filtered through a pad of celite and concentrated to obtain crude product. The crude product was washed with n-pentane and dried to afford tert-butyl (((3S)-1-(3-(2,6-dioxopiperidin-3-yl)-1-methyl-1H-indazol-6-yl)piperidin-3-yl)methyl) carbamate (0.32 g) as a brown solid.

LC-MS (ESI): m/z=456.58 [M+H]⁺.

### Preparation of 3-(6-((S)-3-(aminomethyl)piperidin-1-yl)-1-methyl-1H-indazol-3-yl) piperidine-2,6-dione

A stirred solution of tert-butyl (((3S)-1-(3-(2,6-dioxopiperidin-3-yl)-1-methyl-1H-indazol-6-yl)piperidin-3-yl)methyl) carbamate (0.32 g, 0.70 mmol)) in DCM (3.2 mL) was cooled to 0° C. TFA (2.5 mL) was added and the reaction mixture allowed to warm to room temperature over 2 hours with stirring. The reaction mixture was concentrated to obtain crude product. The crude product was washed with n-pentane to afford 3-(6-((S)-3-(aminomethyl)piperidin-1-yl)-1-methyl-1H-indazol-3-yl)piperidine-2,6-dione (0.22 g) as a brown solid. LC-MS (ESI): m/z=356.46 [M+H]⁺.

### Preparation of 2-((6-((5-chloro-2-((((3S)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide

To a stirred solution of 3-(6-((S)-3-(aminomethyl)piperidin-1-yl)-1-methyl-1H-indazol-3-yl) piperidine-2,6-dione (0.25 g, 0.73 mmol) and 2-((6-((2,5-dichloropyrimidin-4-yl)amino)-1-methyl-2-oxo-1,2-dihydroquinolin-3-yl)oxy)-N-methylacetamide (0.12 g, 0.29 mmol) in DMSO, N,N-diisopropylethylamine (0.76 g, 5.86 mmol) was added. The reaction mixture was stirred at 100° C. for 6 hours. The reaction mixture was poured into ice-cold water and stirred for 15 minutes. The resulting precipitate was filtered and dried to obtain crude product. The crude product was purified by prep-HPLC to afford 2-((6-((5-chloro-2-((((3S)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide (0.94 mg) as a light pink solid.

LC-MS (ESI): m/z=727.52 [M+H]⁺.

¹H NMR (400 MHZ, DMSO-d₆): δ 10.84 (s, 1H), 8.20 (s, 1H), 8.04-7.80 (m, 4H), 7.38 (br s, 2H), 7.09-7.04 (m, 2H), 6.87-6.72 (m, 2H), 4.53 (s, 2H), 4.25-4.21 (m, 1H), 3.83 (s, 3H), 3.56-3.50 (br, 6H), 3.21-3.15 (m, 2H), 2.66-2.51 (m, 6H), 2.32-2.25 (m, 1H), 2.17-2.08 (m, 1H), 1.98-1.92 (m, 1H), 1.73 (br, 2H), 1.52-1.49 (m, 1H), 1.29-1.27 (m, 1H), 1.82-1.77 (m, 1H).

### Example 42. Synthesis of 2-((6-((5-chloro-2-((((3R)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide (Compound 295a)

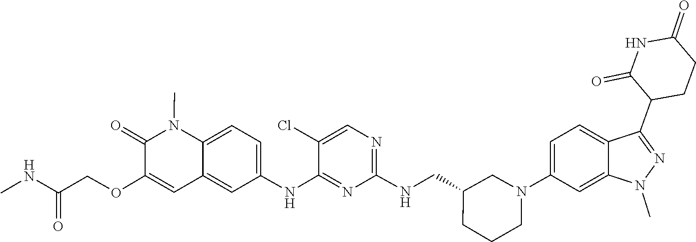 [SMILES: CNC(=O)COc1cc2cc(Nc3nc(NC[C@H]4CCCN(c5ccc6c(C7CCC(=O)NC7=O)nn(C)c6c5)C4)ncc3Cl)ccc2n(C)c1=O]

### Preparation of 2-((6-((5-chloro-2-((((3R)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide

2-((6-((5-chloro-2-((((3R)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide (54 mg) was prepared as a brown solid in a similar manner as 2-((6-((5-chloro-2-((((3S)-1-(3-(2,6-dioxopiperidin-3-yl)-1-methyl-1H-indazol-6-yl)piperidin-3-yl)methyl)amino)pyrimidin-4-yl)amino)-1-methyl-2-oxo-1,2-dihydroquinolin-3-yl)oxy)-N-methylacetamide (see, e.g., Example 41).

LC-MS (ESI): m/z=727.52 [M+H]⁺.

¹H NMR (400 MHZ, DMSO-d₆): δ 10.84 (s, 1H), 8.72 (s, 1H), 8.03-7.91 (m, 3H), 7.82-7.80 (m, 1H), 7.39 (br s, 2H), 7.18 (br s, 2H), 6.73 (br s, 2H), 4.53 (br s, 1H), 4.25-4.22 (m, 1H), 3.83 (s, 3H), 3.60-3.58 (m, 6H), 3.19 (br, 2H), 2.65-2.51 (m, 6H), 2.32-2.26 (m, 1H), 2.17-2.13 (m, 1H), 1.93-1.90 (m, 1H), 1.76-1.73 (m, 2H), 1.53-1.51 (m, 1H), 1.14-1.11 (m, 1H).

### Example 43. Synthesis of 2-((6-((5-chloro-2-(4-(3-((3-(2,6-dioxopiperidin-3-yl)-1-methyl-1H-indazol-6-yl)oxy) propyl)piperidin-1-yl)pyrimidin-4-yl)amino)-1-methyl-2-oxo-1,2-dihydroquinolin-3-yl)oxy)-N-methylacetamide (Compound 296)

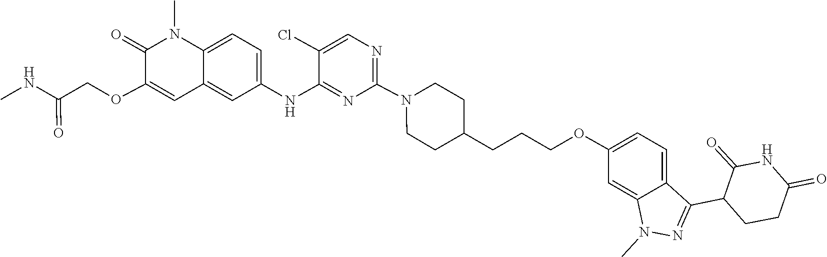 [SMILES: CNC(=O)COc1cc2cc(Nc3nc(N4CCC(CCCOc5ccc6c(C7CCC(=O)NC7=O)nn(C)c6c5)CC4)ncc3Cl)ccc2n(C)c1=O]

### Preparation of tert-butyl 4-(3-((3-(2,6-bis(benzyloxy)pyridin-3-yl)-1-methyl-1H-indazol-6-yl)oxy) propyl)piperidine-1-carboxylate

To a stirred solution of tert-butyl 4-(3-hydroxypropyl)piperidine-1-carboxylate (1.0 g, 4.11 mmol) in 1,4-dioxane (20.0 mL), 3-(2,6-bis(benzyloxy)pyridin-3-yl)-6-bromo-1-methyl-1H-indazole (1.0 g, 2.05 mmol) was added. The reaction purged with nitrogen for 15 minutes. Potassium tert-butoxide (2.93 g, 9.01 mmol), Ruphos (0.28 g, 0.6 mmol), and Ruphos Pd G3 (0.69 g, 6.16 mmol) were added, and the reaction was stirred at room temperature for 16 hours. The reaction mixture was quenched with water and extracted into ethyl acetate. The combined organic layers were washed with brine solution, dried over anhydrous Na₂SO₄, filtered, and concentrated to obtain crude product. The crude product was purified by flash column chromatography (SiO₂, 60-120 mesh, 45% ethyl acetate in petroleum ether) to afford tert-butyl 4-(3-((3-(2,6-bis(benzyloxy)pyridin-3-yl)-1-methyl-1H-indazol-6-yl)oxy) propyl)piperidine-1-carboxylate (0.55 g) as a yellow solid.

LC-MS (ESI): m/z=663.34 [M+H]⁺.

### Preparation of tert-butyl 4-(3-((3-(2,6-dioxopiperidin-3-yl)-1-methyl-1H-indazol-6-yl)oxy) propyl)piperidine-1-carboxylate

To a stirred solution of tert-butyl 4-(3-((3-(2,6-bis(benzyloxy)pyridin-3-yl)-1-methyl-1H-indazol-6-yl)oxy) propyl)piperidine-1-carboxylate (0.50 g, 0.75 mmol) in THF (20 mL), 20% Pd(OH)₂ on carbon, moisture 50% wet (0.50 g) was added. The reaction was put under hydrogen atmosphere (balloon pressure) and stirred at room temperature for 5 hours. The reaction mixture was diluted with EtOAc and filtered through a pad of celite. The celite was washed with 30% THF in EtOAc. The collected filtrate was concentrated under vacuum to obtain tert-butyl 4-(3-((3-(2,6-dioxopiperidin-3-yl)-1-methyl-1H-indazol-6-yl)oxy) propyl)piperidine-1-carboxylate (0.25 g) as an off-white solid.

LC-MS (ESI): m/z=429.44 [M-tBu]⁺.

### Preparation of 3-(1-methyl-6-(3-(piperidin-4-yl) propoxy)-1H-indazol-3-yl)piperidine-2,6-dione hydrochloride

To a stirred solution of tert-butyl 4-(3-((3-(2,6-dioxopiperidin-3-yl)-1-methyl-1H-indazol-6-yl)oxy) propyl)piperidine-1-carboxylate (0.25 g, 0.51 mmol) in DCM (6.5 mL), 4 M HCl in dioxane (3.25 mL) was added. The reaction was stirred at room temperature for 16 hours. The reaction mixture was concentrated under reduced pressure to obtain crude product. The crude product was triturated with diethyl ether to afford 3-(1-methyl-6-(3-(piperidin-4-yl) propoxy)-1H-indazol-3-yl)piperidine-2,6-dione hydrochloride (0.2 g).

LC-MS (ESI): m/z=385.49 [M+H]⁺.

### Preparation of 2-((6-((5-chloro-2-(4-(3-((3-(2,6-dioxopiperidin-3-yl)-1-methyl-1H-indazol-6-yl)oxy) propyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(3-(piperidin-4-yl) propoxy)-1H-indazol-3-yl)piperidine-2,6-dione hydrochloride (0.20 g, 0.44 mmol) in DMSO (4.0 mL), 2-((6-((2,5-dichloropyrimidin-4-yl)amino)-1-methyl-2-oxo-1,2-dihydroquinolin-3-yl)oxy)-N-methylacetamide (0.13 g, 0.33 mmol) and DIPEA (0.43 mL, 2.37 mmol) were added. The reaction was heated to 120° C. for 6 hours. The reaction mixture was quenched in ice-cold water. The resulting precipitate was filtered and dried to obtain crude product. The crude product was purified by preparative HPLC to afford 2-((6-((5-chloro-2-(4-(3-((3-(2,6-dioxopiperidin-3-yl)-1-methyl-1H-indazol-6-yl)oxy) propyl)piperidin-1-yl)pyrimidin-4-yl)amino)-1-methyl-2-oxo-1,2-dihydroquinolin-3-yl)oxy)-N-methylacetamide (0.018 g) as an off-white solid.

LC-MS (ESI): m/z=756.49 [M+H]⁺.

¹H NMR (400 MHZ, DMSO-d₆): δ 10.85 (s, 1H), 8.81 (s, 1H), 8.03 (s, 1H), 7.96-7.93 (m, 2H), 7.76-7.73 (m, 1H), 7.55-7.53 (d, J=8.8 Hz, 1H), 7.48-7.46 (d, J=9.2 Hz, 1H), 7.09 (s, 1H), 7.038-7.034 (d, J=1.6 Hz, 1H), 6.73-6.70 (m, 1H), 4.57 (s, 2H), 4.51-4.48 (d, J=12.4 Hz, 2H), 4.30-4.26 (m, 1H), 4.06-4.03 (t, J-6.4 Hz, 2H), 3.91 (s, 3H), 3.67 (s, 3H), 2.86-2.80 (t, J=12 Hz, 2H), 2.66-2.61 (m, 4H), 2.32-2.29 (m, 1H), 2.18-2.16 (m, 1H), 1.83-1.79 (m, 6H), 1.75-1.68 (m, 1H), 1.42-1.40 (m, 2H), 1.38-1.25 (m, 2H), 1.08-1.06 (m, 2H).

### Example 44. Synthesis of 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (Compound 297a)

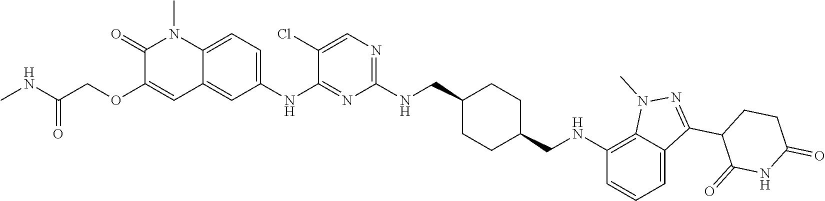 [SMILES: CNC(=O)COc1cc2cc(Nc3nc(NC[C@H]4CC[C@@H](CNc5cccc6c(C7CCC(=O)NC7=O)nn(C)c56)CC4)ncc3Cl)ccc2n(C)c1=O]

### Preparation of tert-butyl (((1s,4s)-4-(((3-(2,6-bis(benzyloxy)pyridin-3-yl)-1-methyl-1H-indazol-7-yl)amino)methyl)cyclohexyl)methyl) carbamate

A stirred solution of tert-butyl (((1s,4s)-4-(aminomethyl)cyclohexyl)methyl) carbamate (0.2 g, 0.82 mmol), 3-(2,6-bis(benzyloxy)pyridin-3-yl)-7-bromo-1-methyl-1H-indazole (0.41 g, 0.82 mmol), cesium carbonate (1.07 g, 3.30 mmol) in 1,4-dioxane (4 mL) was degassed using nitrogen for 15 minutes. Pd-PEPPSI-IHeptCl (0.04 g, 0.04 mmol) was added, and the reaction was heated to 100° C. for 16 hours. The reaction mixture was filtered through a pad of celite. The celite was washed with DCM (100 mL), and the filtrate was concentrated under reduced pressure to obtain crude product. The crude product was purified by flash column chromatography (SiO₂, 100-200 mesh, 25% ethyl acetate in petroleum ether as eluent) to afford tert-butyl (((1s,4s)-4-(((3-(2,6-bis(benzyloxy)pyridin-3-yl)-1-methyl-1H-indazol-7-yl)amino)methyl)cyclohexyl)methyl) carbamate (0.20 g) as a brown solid.

LC-MS (ESI): m/z=662.37 [M+H]⁺.

### Preparation of tert-butyl (((1s,4s)-4-(((3-(2,6-dioxopiperidin-3-yl)-1-methyl-1H-indazol-7-yl)amino)methyl)cyclohexyl)methyl) carbamate

To stirred solution of tert-butyl (((1s,4s)-4-(((3-(2,6-bis(benzyloxy)pyridin-3-yl)-1-methyl-1H-indazol-7-yl)amino)methyl)cyclohexyl)methyl) carbamate (0.25 g, 0.37 mmol) in THF (15 mL), 20% Pd(OH)₂/C (100% w/w)(0.26 g) was added. The reaction was put under hydrogen atmosphere (15 psi) and stirred at room temperature for 3 hours. The reaction mixture was filtered through a pad of celite. The celite was washed with 30% THF:DCM (600 mL). The filtrate was concentrated under reduced pressure to obtain crude product. The crude product was purified by flash column chromatography (SiO₂, 100-200, 60% ethyl acetate in pet ether as eluent) to afford tert-butyl (((1s,4s)-4-(((3-(2,6-dioxopiperidin-3-yl)-1-methyl-1H-indazol-7-yl)amino)methyl)cyclohexyl)methyl) carbamate (0.14 g) as a brown semi-solid.

LC-MS (ESI): m/z=506.56 [M+Na]⁺.

### Preparation of 3-(7-((((1s,4s)-4-(aminomethyl)cyclohexyl)methyl)amino)-1-methyl-1H-indazol-3-yl)piperidine-2,6-dione hydrochloride

To a stirred solution of tert-butyl (((1s,4s)-4-(((3-(2,6-dioxopiperidin-3-yl)-1-methyl-1H-indazol-7-yl)amino)methyl)cyclohexyl)methyl) carbamate (0.13 g, 0.21 mmol) in DCM (2.6 mL), 4 M HCl in 1,4-dioxane (1.3 mL) was added at 0° C. The reaction was allowed to warm to room temperature over 3 hours with stirring. The reaction mixture concentrated under reduced pressure to obtain crude product. The crude product was purified by trituration with pentane (10 mL) to afford 3-(7-((((1s,4s)-4-(aminomethyl)cyclohexyl)methyl)amino)-1-methyl-1H-indazol-3-yl)piperidine-2,6-dione hydrochloride (0.17 g) as a white solid.

LC-MS (ESI): m/z=384.48 [M+H]⁺.

### Preparation of 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide

To a stirred solution of 3-(7-((((1s,4s)-4-(aminomethyl)cyclohexyl)methyl)amino)-1-methyl-1H-indazol-3-yl)piperidine-2,6-dione hydrochloride (0.1 g, 0.23 mmol) and 2-((6-((2,5-dichloropyrimidin-4-yl)amino)-1-methyl-2-oxo-1,2-dihydroquinolin-3-yl)oxy)-N-methylacetamide (0.05 g, 0.12 mmol) in DMSO (2 mL), DIPEA (0.2 mL, 1.19 mmol) and p-toluene sulfonic acid monohydrate (0.002 g, 0.05 mmol) were added. The reaction was heated to 100° C. for 4 hours. The solvent was removed using a centrifugal evaporator to obtain crude product. The crude product was purified by prep-HPLC to afford 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (0.026 g) as an off-white solid.

LC-MS (ESI): m/z=755.53 [M+H]⁺.

¹H NMR (400 MHZ, DMSO-d₆): δ 10.87 (s, 1H), 8.68 (s, 1H), 8.04 (br s, 1H), 7.94 (br s, 2H), 7.83 (d, J=7.60 Hz, 1H), 7.43 (d, J=9.20 Hz, 1H), 7.20 (br s, 1H), 7.10-7.05 (m, 1H), 6.93 (d, J=8.00 Hz, 1H), 6.85 (br s, 1H), 6.26 (br s, 1H), 5.25-5.13 (m, 1H), 4.55 (s, 2H), 4.23-4.21 (m, 4H), 3.64 (s, 3H), 3.21-3.20 (m, 2H), 3.03-2.94 (m, 1H), 2.81-2.71 (m, 1H), 2.66 (d, J=4.80 Hz, 3H), 2.62-2.61 (m, 2H), 2.27-2.25 (m, 1H), 2.13-2.11 (m, 1H), 1.75-1.78 (m, 2H), 1.52-1.48 (m, 8H).

### Example 45. Synthesis of 2-((6-((5-chloro-2-((3R)-3-(((3-(2,6-dioxopiperidin-3-yl)-1-methyl-1H-indazol-7-yl)(methyl)amino)methyl)piperidin-1-yl)pyrimidin-4-yl)amino)-1-methyl-2-oxo-1,2-dihydroquinolin-3-yl)oxy)-N-methylacetamide (Compound 299a)

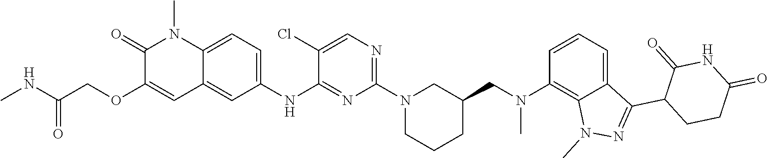 [SMILES: CNC(=O)COc1cc2cc(Nc3nc(N4CCC[C@H](CN(C)c5cccc6c(C7CCC(=O)NC7=O)nn(C)c56)C4)ncc3Cl)ccc2n(C)c1=O]

### Preparation of tert-butyl (R)-3-(((3-(2,6-bis(benzyloxy)pyridin-3-yl)-1-methyl-1H-indazol-7-yl)(methyl)amino)methyl)piperidine-1-carboxylate

To a stirred solution of tert-butyl (R)-3-(((3-(2,6-bis(benzyloxy)pyridin-3-yl)-1-methyl-1H-indazol-7-yl)amino)methyl)piperidine-1-carboxylate (0.25 g, 0.39 mmol) in DMF (5.0 mL), NaH (0.02 g, 0.98 mmol) was added at 0° C., and the reaction was stirred for 30 minutes. Methyl iodide (0.04 mL, 0.59 mmol was added, and the reaction was allowed to warm to room temperature over 2 hours with stirring. The reaction mixture was diluted with ethyl acetate (50 mL) and quenched with ice-cold water (5 mL). The separated organic layer was dried over anhydrous Na₂SO₄, filtered, and concentrated to obtain tert-butyl (R)-3-(((3-(2,6-bis(benzyloxy)pyridin-3-yl)-1-methyl-1H-indazol-7-yl)(methyl)amino)methyl)piperidine-1-carboxylate (0.19 g) as an off-white semi-solid.

LC-MS (ESI): m/z=648.77 [M+H]⁺.

### Preparation of tert-butyl (3R)-3-(((3-(2,6-dioxopiperidin-3-yl)-1-methyl-1H-indazol-7-yl)(methyl)amino)methyl)piperidine-1-carboxylate

To a stirred solution of tert-butyl (R)-3-(((3-(2,6-bis(benzyloxy)pyridin-3-yl)-1-methyl-1H-indazol-7-yl)(methyl)amino)methyl)piperidine-1-carboxylate (0.31 g, 0.48 mmol), AcOH (0.03 g, 0.48 mmol), and DMF (0.90 mL) in THF (9.1 mL), 10% Pd(OH)₂/C (0.27 g, 1.94 mmol) was added. The reaction was put under hydrogen (15 psi) and stirred at room temperature for 20 hours. The reaction mixture was diluted with THF (30 mL) and filtered through a pad of celite. The celite was washed with 20% THF in DCM (150 mL). The filtrate was concentrated under vacuum to afford tert-butyl (3R)-3-(((3-(2,6-dioxopiperidin-3-yl)-1-methyl-1H-indazol-7-yl)(methyl)amino)methyl)piperidine-1-carboxylate (0.18 g) as a pale brown semi-solid.

LC-MS (ESI): m/z=470.55 [M+H]⁺.

### Preparation of 3-(1-methyl-7-(methyl(((S)-piperidin-3-yl)methyl)amino)-1H-indazol-3-yl)piperidine-2,6-dione

To a stirred solution of tert-butyl (3R)-3-(((3-(2,6-dioxopiperidin-3-yl)-1-methyl-1H-indazol-7-yl)(methyl)amino)methyl)piperidine-1-carboxylate (0.14 g, 0.29 mmol) in DCM (1.40 mL), TFA (0.70 mL) was added at 0° C. The reaction was allowed to warm to room temperature over 0.5 hours with stirring. The reaction mixture was concentrated under vacuum to afford 3-(1-methyl-7-(methyl(((S)-piperidin-3-yl)methyl)amino)-1H-indazol-3-yl)piperidine-2,6-dione (0.11 g) as a pale green semi-solid.

LC-MS (ESI): m/z=370.42 [M+H]⁺.

### Preparation of 2-((6-((5-chloro-2-((3R)-3-(((3-(2,6-dioxopiperidin-3-yl)-1-methyl-1H-indazol-7-yl)(methyl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-7-(methyl(((S)-piperidin-3-yl)methyl)amino)-1H-indazol-3-yl)piperidine-2,6-dione (0.11 g, 0.30 mmol) in DMSO (2.2 mL), 2-((6-((2,5-dichloropyrimidin-4-yl)amino)-1-methyl-2-oxo-1,2-dihydroquinolin-3-yl)oxy)-N-methylacetamide (0.049 g, 0.12 mmol) and DIPEA (0.7 mL, 3.57 mmol) were added. The reaction was heated at 120° C. for 7 hours. The reaction mixture was poured into water (10 mL). The precipitated solid was filtered and dried under vacuum to obtain crude product. The crude product was purified by preparative HPLC to afford 2-((6-((5-chloro-2-((3R)-3-(((3-(2,6-dioxopiperidin-3-yl)-1-methyl-1H-indazol-7-yl)(methyl)amino)methyl)piperidin-1-yl)pyrimidin-4-yl)amino)-1-methyl-2-oxo-1,2-dihydroquinolin-3-yl)oxy)-N-methylacetamide (0.029 g) as an off-white solid.

LC-MS (ESI): m/z=741.49 [M+H]⁺.

¹H NMR (400 MHZ, DMSO-d₆): δ 10.95 (s, 1H), 8.81 (s, 1H), 7.97 (s, 1H), 7.93-7.91 (m, 2H), 7.74 (d, J=8.80 Hz, 1H), 7.46 (d, J=9.20 Hz, 1H), 7.37 (d, J=7.60 Hz, 1H), 7.14 (s, 1H), 6.99-6.98 (m, 2H), 4.54 (s, 3H), 4.34-4.20 (m, 5H), 3.63 (s, 3H), 2.97-2.91 (m, 3H), 2.80-2.74 (m, 1H), 2.67-2.62 (m, 8H), 2.33-2.30 (m, 1H), 2.18-2.16 (m, 1H), 1.84-1.82 (m, 1H), 1.75-1.66 (m, 2H), 1.47-1.38 (m, 2H).

### Example 46. Synthesis of 2-((6-((5-chloro-2-(4-((1-(3-(2,6-dioxopiperidin-3-yl)-1-methyl-1H-indazol-7-yl) azetidin-3-yl)methyl)piperidin-1-yl)pyrimidin-4-yl)amino)-1-methyl-2-oxo-1,2-dihydroquinolin-3-yl)oxy)-N-methylacetamide (Compound 310)

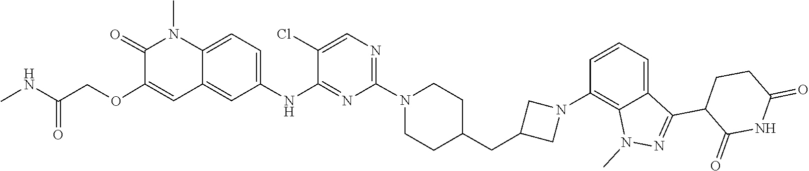 [SMILES: CNC(=O)COc1cc2cc(Nc3nc(N4CCC(CC5CN(c6cccc7c(C8CCC(=O)NC8=O)nn(C)c67)C5)CC4)ncc3Cl)ccc2n(C)c1=O]

### Preparation of 2-((6-((5-chloro-2-(4-((1-(3-(2,6-dioxopiperidin-3-yl)-1-methyl-1H-indazol-7-yl) azetidin-3-yl)methyl)piperidin-1-yl)pyrimidin-4-yl)amino)-1-methyl-2-oxo-1,2-dihydroquinolin-3-yl)oxy)-N-methylacetamide

2-((6-((5-chloro-2-(4-((1-(3-(2,6-dioxopiperidin-3-yl)-1-methyl-1H-indazol-7-yl) azetidin-3-yl)methyl)piperidin-1-yl)pyrimidin-4-yl)amino)-1-methyl-2-oxo-1,2-dihydroquinolin-3-yl)oxy)-N-methylacetamide (2.6 mg) was prepared as a white solid in a similar manner as 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydroquinolin-3-yl)oxy)-N-methylacetamide (see, e.g., Example 21).

LC-MS (ESI): m/z=767.51 [M+H]⁺.

¹H NMR (400 MHZ, DMSO-d₆): δ 10.86 (s, 1H), 8.82 (s, 1H), 8.03 (s, 1H), 7.96-7.93 (m, 2H), 7.76-7.74 (d, J=9.2 Hz, 1H), 7.48-7.46 (d, J=9.2 Hz, 1H), 7.21-7.19 (d, J=8.0 Hz, 1H), 7.09 (s, 1H), 6.99-6.95 (t, J=15.6 Hz, J=7.6 Hz, 1H), 6.71-6.69 (d, J=7.2 Hz, 1H) 4.57-4.47 (m, 4H), 4.32-4.28 (m, 1H), 4.16 (s, 3H), 4.02-4.00 (d, J=7.2 Hz, 2H), 3.67 (s, 3H) 3.49-3.46 (m, 3H), 2.84-2.78 (t, J=23.2 Hz, J=12.0 Hz, 1H), 2.66-2.60 (m, 5H), 2.32 (m, 1H), 2.16 (m, 1H), 1.71-1.68 (m, 2H), 1.56 (m, 3H), 1.23 (s, 2H) and 1.06 (m, 2H).

### Example 47. Synthesis of 2-((6-((5-chloro-2-((2R)-2-(((3-(2,6-dioxopiperidin-3-yl)-1-methyl-1H-indazol-7-yl)amino)methyl)pyrrolidin-1-yl)pyrimidin-4-yl)amino)-1-methyl-2-oxo-1,2-dihydroquinolin-3-yl)oxy)-N-methylacetamide (Compound 300a)

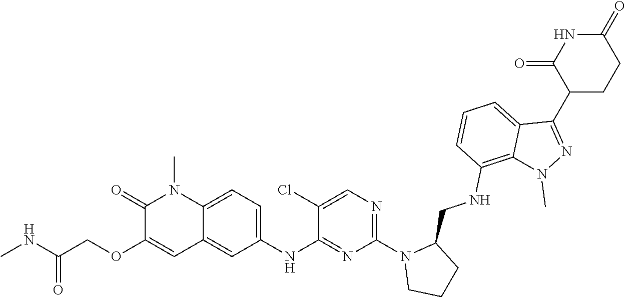 [SMILES: CNC(=O)COc1cc2cc(Nc3nc(N4CCC[C@@H]4CNc4cccc5c(C6CCC(=O)NC6=O)nn(C)c45)ncc3Cl)ccc2n(C)c1=O]

### Preparation of 2-((6-((5-chloro-2-((2R)-2-(((3-(2,6-dioxopiperidin-3-yl)-1-methyl-1H-indazol-7-yl)amino)methyl)pyrrolidin-1-yl)pyrimidin-4-yl)amino)-1-methyl-2-oxo-1,2-dihydroquinolin-3-yl)oxy)-N-methylacetamide

2-((6-((5-chloro-2-((2R)-2-(((3-(2,6-dioxopiperidin-3-yl)-1-methyl-1H-indazol-7-yl)amino)methyl)pyrrolidin-1-yl)pyrimidin-4-yl)amino)-1-methyl-2-oxo-1,2-dihydroquinolin-3-yl)oxy)-N-methylacetamide (31.0 mg) was prepared as an off-white solid in a similar manner as 2-((6-((5-chloro-2-((3R)-3-(((3-(2,6-dioxopiperidin-3-yl)-1-methyl-1H-indazol-7-yl)amino)methyl)piperidin-1-yl)pyrimidin-4-yl)amino)-1-methyl-2-oxo-1,2-dihydroquinolin-3-yl)oxy)-N-methylacetamide (see, e.g., Example 14).

LC-MS (ESI): m/z=713.50 [M+H]⁺.

¹H NMR (400 MHZ, DMSO-d₆): δ 10.85 (s, 1H), 8.82 (s, 1H), 8.15-7.87 (m, 4H), 7.60-7.10 (m, 2H), 6.93-5.08 (m, 3H), 4.57-4.25 (m, 6H), 3.67-3.47 (m, 4H), 3.16-3.03 (m, 2H), 2.89-2.61 (m, 8H), 2.32-1.95 (m, 6H).

### Example 48. Synthesis of 2-((6-((5-chloro-2-(4-(((3R)-1-(3-(2,6-dioxopiperidin-3-yl)-1-methyl-1H-indazol-7-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (Compound 305a)

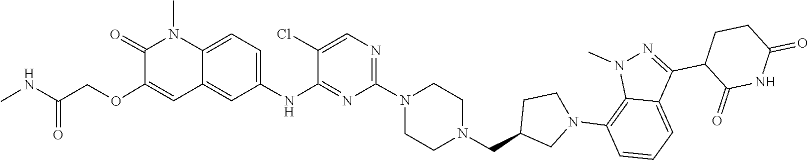 [SMILES: CNC(=O)COc1cc2cc(Nc3nc(N4CCN(C[C@H]5CCN(c6cccc7c(C8CCC(=O)NC8=O)nn(C)c67)C5)CC4)ncc3Cl)ccc2n(C)c1=O]

### Preparation of tert-butyl(S)-4-(1-((benzyloxy)carbonyl)pyrrolidine-3-carbonyl)piperazine-1-carboxylate

To a stirred solution of(S)-1-((benzyloxy)carbonyl)pyrrolidine-3-carboxylic acid (3.0 g, 12.03 mmol) and tert-butyl piperazine-1-carboxylate (2.69 g, 14.44 mmol) in DMF (30.0 mL), N,N-diisopropylethylamine (12.6 mL, 72.21 mmol) was added at 0° C. After stirring for 10 minutes, T3P (11.5 mL, 36.10 mmol) was added. The resultant reaction mixture was allowed to warm to room temperature over 4 hours with stirring. The reaction mixture was quenched with ice water (50 mL) and stirred for 1 hour. The precipitated solid was filtered and dried under vacuum to obtain crude product. The crude product was triturated with n-pentane (3×20 mL) to afford tert-butyl(S)-4-(1-((benzyloxy)carbonyl)pyrrolidine-3-carbonyl)piperazine-1-carboxylate (5.0 g) as an off-white solid.

LC-MS (ESI): m/z=440.55 [M+Na]⁺.

### Preparation of tert-butyl (R)-4-((1-((benzyloxy)carbonyl)pyrrolidin-3-yl)methyl)piperazine-1-carboxylate

To a stirred solution of tert-butyl(S)-4-(1-((benzyloxy)carbonyl)pyrrolidine-3-carbonyl)piperazine-1-carboxylate (5.0 g, 11.97 mmol) in THF (50 mL), borane dimethyl sulfide (20 mL) was added at 0° C. The reaction was allowed to warm to room temperature over 6 hours with stirring. The reaction mixture was quenched with methanol (100 mL) at 0° C. The reaction was concentrated under vacuum to afford tert-butyl (R)-4-((1-((benzyloxy)carbonyl)pyrrolidin-3-yl)methyl)piperazine-1-carboxylate (4.0 g) as a colorless gummy solid.

¹H NMR (400 MHZ, DMSO-d₆): δ 7.41-7.29 (s, 5H), 5.05 (s, 2H), 3.55-3.23 (m, 4H), 3.02-2.96 (m, 4H), 2.35-2.25 (m, 5H), 1.93-1.86 (m, 1H), 1.56-1.54 (m, 2H), 1.38 (d, J=12.80 Hz, 10H).

### Preparation of tert-butyl(S)-4-(pyrrolidin-3-ylmethyl)piperazine-1-carboxylate

A stirred solution of tert-butyl (R)-4-((1-((benzyloxy)carbonyl)pyrrolidin-3-yl)methyl)piperazine-1-carboxylate (2.50 g, 6.19 mmol) in THF (50 mL) was degassed with nitrogen for 5 minutes. 10% palladium hydroxide on carbon (2.50 g, 100% w/w) was added. The reaction was put under hydrogen atmosphere (15 psi) and stirred at room temperature for 16 hours. The reaction was filtered through a pad of celite. The celite was washed with 50% THF:DCM (200 mL). The filtrate was concentrated and dried under vacuum to afford tert-butyl(S)-4-(pyrrolidin-3-ylmethyl)piperazine-1-carboxylate (1.60 g).

LC-MS (ESI): m/z=270.37 [M+H]⁺.

### Preparation of tert-butyl (R)-4-((1-(3-(2,6-bis(benzyloxy)pyridin-3-yl)-1-methyl-1H-indazol-7-yl)pyrrolidin-3-yl)methyl)piperazine-1-carboxylate

To a stirred solution of 3-(2,6-bis(benzyloxy)pyridin-3-yl)-7-bromo-1-methyl-1H-indazole (0.80 g, 1.59 mmol) and tert-butyl(S)-4-(pyrrolidin-3-ylmethyl)piperazine-1-carboxylate (0.65 g, 2.39 mmol) in 1,4-dioxane (16.0 mL), cesium carbonate (1.56 g, 4.79 mmol) was added. The reaction was degassed with argon for 15 minutes. Pd-PEPPSI IHeptCl was added, and the reaction mixture was stirred at 100° C. for 16 hours. The reaction mixture was cooled to room temperature and diluted with ethyl acetate (30 mL). The reaction was filtered through a pad of celite, and the celite was washed with 10% MeOH:DCM (100 mL). The filtrate was concentrated to obtain crude product. The crude product was purified by column chromatogram (SiO₂, 230-400 mesh 20-30% EtOAc in petroleum ether) to afford tert-butyl (R)-4-((1-(3-(2,6-bis(benzyloxy)pyridin-3-yl)-1-methyl-1H-indazol-7-yl)pyrrolidin-3-yl)methyl)piperazine-1-carboxylate (0.58 g) as a pale yellow solid.

LC-MS (ESI): m/z=689.73 [M+H]⁺.

### Preparation of tert-butyl 4-(((3R)-1-(3-(2,6-dioxopiperidin-3-yl)-1-methyl-1H-indazol-7-yl)pyrrolidin-3-yl)methyl)piperazine-1-carboxylate

To a stirred solution of tert-butyl (R)-4-((1-(3-(2,6-bis(benzyloxy)pyridin-3-yl)-1-methyl-1H-indazol-7-yl)pyrrolidin-3-yl)methyl)piperazine-1-carboxylate (0.58 g, 0.84 mmol) in THF (11.0 mL), 10% palladium hydroxide on carbon (1.16 g, 200% w/w) was added. The reaction was put under a hydrogen atmosphere (15 psi) and stirred at room temperature for 16 hours. The reaction mixture was diluted with THF (50 mL) and filtered through a pad of celite. The celite was washed with 150 mL of THF:DCM (1:1). The filtrate was concentrated under reduced pressure to afford tert-butyl 4-(((3R)-1-(3-(2,6-dioxopiperidin-3-yl)-1-methyl-1H-indazol-7-yl) 278pyrrolidine-3-yl)methyl)piperazine-1-carboxylate (0.45 g) as an off-white solid.

LC-MS (ESI): m/z=511.59 [M+H]⁺.

### Preparation of 3-(1-methyl-7-((R)-3-(piperazin-1-ylmethyl)pyrrolidin-1-yl)-1H-indazol-3-yl)piperidine-2,6-dione

To a stirred solution of tert-butyl 4-(((3R)-1-(3-(2,6-dioxopiperidin-3-yl)-1-methyl-1H-indazol-7-yl)pyrrolidin-3-yl)methyl)piperazine-1-carboxylate (0.40 g, 0.78 mmol) in DCM (8.0 mL), TFA (4.0 mL, 10) was added at 0° C. The reaction was allowed to warm to room temperature over 3 hours with stirring. The reaction mixture was concentrated under vacuum and co-distilled with DCM (3×15 mL) to afford 3-(1-methyl-7-((R)-3-(piperazin-1-ylmethyl)pyrrolidin-1-yl)-1H-indazol-3-yl)piperidine-2,6-dione (0.41 g) as the TFA salt.

LC-MS (ESI): m/z=411.48 [M+H]⁺.

### Preparation of 2-((6-((5-chloro-2-(4-(((3R)-1-(3-(2,6-dioxopiperidin-3-yl)-1-methyl-1H-indazol-7-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-7-((R)-3-(piperazin-1-ylmethyl)pyrrolidin-1-yl)-1H-indazol-3-yl)piperidine-2,6-dione (0.40 g, 0.97 mmol) in DMSO (4.0 mL), DIPEA (1.4 mL, 7.80 mmol) and 2-((6-((2,5-dichloropyrimidin-4-yl)amino)-1-methyl-2-oxo-1,2-dihydroquinolin-3-yl)oxy)-N-methylacetamide (0.28 g, 0.68 mmol) were added. The reaction mixture was stirred at 100° C. for 16 hours. The reaction mixture was quenched with ice water and stirred for 15 minutes. The precipitated solid was filtered and dried to obtain crude product. The crude product was purified by prep-HPLC to afford 2-((6-((5-chloro-2-(4-(((3R)-1-(3-(2,6-dioxopiperidin-3-yl)-1-methyl-1H-indazol-7-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (0.065 g) as an off-white solid.

LC-MS (ESI): m/z=782.55 [M+H]⁺.

¹H NMR (400 MHZ, DMSO-d₆): δ 10.86 (s, 1H), 8.86 (s, 1H), 8.05 (s, 1H), 7.95-7.93 (m, 2H), 7.74 (d, J=2.00 Hz, 1H), 7.47 (d, J=9.20 Hz, 1H), 7.34 (d, J=7.60 Hz, 1H), 7.12 (s, 1H), 7.04-6.98 (m, 2H), 4.58 (s, 2H), 4.33 (q, J=5.20 Hz, 1H), 4.19 (s, 3H), 3.67-3.59 (m, 7H), 3.17-2.96 (m, 4H), 2.67-2.61 (m, 6H), 2.50-2.13 (m, 9H), 1.63-1.63 (m, 1H).

### Example 49. Synthesis of 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (Compound 306a)

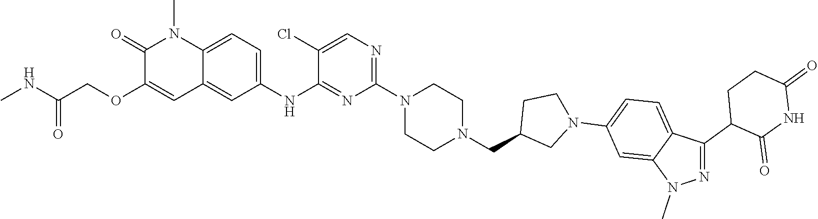 [SMILES: CNC(=O)COc1cc2cc(Nc3nc(N4CCN(C[C@H]5CCN(c6ccc7c(C8CCC(=O)NC8=O)nn(C)c7c6)C5)CC4)ncc3Cl)ccc2n(C)c1=O]

### Preparation of tert-butyl (R)-4-((1-(3-(2,6-bis(benzyloxy)pyridin-3-yl)-1-methyl-1H-indazol-6-yl)pyrrolidin-3-yl)methyl)piperazine-1-carboxylate

To a stirred solution of tert-butyl(S)-4-(pyrrolidin-3-ylmethyl)piperazine-1-carboxylate (0.75 g, 2.78 mmol) and 3-(2,6-bis(benzyloxy)pyridin-3-yl)-6-bromo-1-methyl-1H-indazole (0.70 g, 1.39 mmol) in dioxane (7.0 mL), sodium tert-butoxide (0.40 g, 4.16 mmol) was added. The reaction was degassed with argon for 10 minutes. Brettphos Pd-G3 (0.06 g, 6.61 mmol) was added, and the reaction was stirred at 80° C. for 4 hours. The reaction mixture was filtered through a pad of celite, and the filtrate was concentrated to crude product. The crude product was purified by column chromatogram (SiO₂, 230-400 mesh, 35-40% EtOAc in petroleum ether) to afford tert-butyl (R)-4-((1-(3-(2,6-bis(benzyloxy)pyridin-3-yl)-1-methyl-1H-indazol-6-yl)pyrrolidin-3-yl)methyl)piperazine-1-carboxylate (0.55 g) as an off-white solid.

LC-MS (ESI): m/z=689.77 [M+H]⁺.

### Preparation of tert-butyl 4-(((3R)-1-(3-(2,6-dioxopiperidin-3-yl)-1-methyl-1H-indazol-6-yl)pyrrolidin-3-yl)methyl)piperazine-1-carboxylate

A stirred solution of tert-butyl (R)-4-((1-(3-(2,6-bis(benzyloxy)pyridin-3-yl)-1-methyl-1H-indazol-6-yl)pyrrolidin-3-yl)methyl)piperazine-1-carboxylate (0.55 g, 0.79 mmol) in THF (10.0 mL) was degassed with nitrogen for 5 minutes. 20% Pd(OH)₂ (1.10 g, 100% w/w) was added. The reaction was put under hydrogen atmosphere (15 psi) and stirred at room temperature for 16 hours. The reaction mixture was filtered through a pad of celite. The filtrate was concentrated under vacuum to afford tert-butyl 4-(((3R)-1-(3-(2,6-dioxopiperidin-3-yl)-1-methyl-1H-indazol-6-yl)pyrrolidin-3-yl)methyl)piperazine-1-carboxylate (0.35 g) as a brown gummy solid.

LC-MS (ESI): m/z=511.54 [M+H]⁺.

### Preparation of 3-(1-methyl-6-((R)-3-(piperazin-1-ylmethyl)pyrrolidin-1-yl)-1H-indazol-3-yl)piperidine-2,6-dione

To a stirred solution of tert-butyl 4-(((3R)-1-(3-(2,6-dioxopiperidin-3-yl)-1-methyl-1H-indazol-6-yl)pyrrolidin-3-yl)methyl)piperazine-1-carboxylate (0.35 g, 0.68 mmol) in DCM (3.5 mL), TFA (1.75 mL) was added at 0° C. The reaction was allowed to warm to room temperature over 2 hours with stirring. The reaction mixture was concentrated under vacuum and co-distilled with DCM (3×10 mL) to afford 3-(1-methyl-6-((R)-3-(piperazin-1-ylmethyl)pyrrolidin-1-yl)-1H-indazol-3-yl)piperidine-2,6-dione (0.35 g) as a brown gummy solid.

LC-MS (ESI): m/z=411.48 [M+H]⁺.

### Preparation of 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-6-((R)-3-(piperazin-1-ylmethyl)pyrrolidin-1-yl)-1H-indazol-3-yl)piperidine-2,6-dione (0.35 g, 0.85 mmol) in DMSO (3.5 mL), DIPEA (1.10 g, 8.51 mmol) and 2-((6-((2,5-dichloropyrimidin-4-yl)amino)-1-methyl-2-oxo-1,2-dihydroquinolin-3-yl)oxy)-N-methylacetamide (0.27 g, 0.66 mmol) were added. The reaction mixture was stirred at 100° C. for 6 hours. The reaction mixture was quenched with ice water and stirred for 30 minutes. The precipitate was filtered and dried to obtain crude product. The crude product was purified by prep-HPLC to afford 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (0.070 g) as an off-white solid.

LC-MS (ESI): m/z=780.58 [M−H]⁻.

¹H NMR (400 MHZ, DMSO-d₆): δ 10.83 (s, 1H), 8.87 (s, 1H), 8.06 (s, 1H), 7.94 (d, J=2.40 Hz, 2H), 7.74 (dd, J=2.40, 9.20 Hz, 1H), 7.46 (q, J=6.80 Hz, 2H), 7.12 (s, 1H), 6.55 (dd, J=1.20, 9.00 Hz, 1H), 6.34 (s, 1H), 4.58 (s, 2H), 4.22 (q, J=5.20 Hz, 1H), 3.84 (s, 3H), 3.67 (s, 7H), 3.48 (t, J=7.60 Hz, 1H), 3.40-3.32 (m, 2H), 3.07 (t, J=8.80 Hz, 1H), 2.67-2.59 (m, 6H), 2.50-2.39 (m, 6H), 2.30-2.23 (m, 1H), 2.18-2.12 (m, 2H), 1.77-1.72 (m, 1H).

### Example 50. Synthesis of 2-((6-((5-chloro-2-(4-(((3S)-1-(3-(2,6-dioxopiperidin-3-yl)-1-methyl-1H-indazol-7-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (Compound 305b)

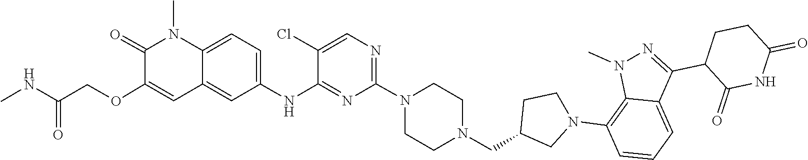 [SMILES: CNC(=O)COc1cc2cc(Nc3nc(N4CCN(C[C@@H]5CCN(c6cccc7c(C8CCC(=O)NC8=O)nn(C)c67)C5)CC4)ncc3Cl)ccc2n(C)c1=O]

### Preparation of tert-butyl (R)-4-(pyrrolidin-3-ylmethyl)piperazine-1-carboxylate

Tert-butyl (R)-4-(pyrrolidin-3-ylmethyl)piperazine-1-carboxylate (0.620 g) was prepared as a pale brown semi-solid in a similar manner as tert-butyl(S)-4-(pyrrolidin-3-ylmethyl)piperazine-1-carboxylate (see, e.g., Example 48).

LC-MS (ESI): m/z=270.37 [M+H]⁺.

### Preparation of tert-butyl(S)-4-((1-(3-(2,6-bis(benzyloxy)pyridin-3-yl)-1-methyl-1H-indazol-7-yl)pyrrolidin-3-yl)methyl)piperazine-1-carboxylate

To a stirred solution of 3-(2,6-bis(benzyloxy)pyridin-3-yl)-7-bromo-1-methyl-1H-indazole (1.0 g, 1.99 mmol) in dioxane (5.0 mL), tert-butyl (R)-4-(pyrrolidin-3-ylmethyl)piperazine-1-carboxylate (2.15 g, 7.99 mmol) and cesium carbonate (1.95 g, 5.99 mmol) were added. The reaction was degassed with argon for 5 minutes. Ruphos (0.09 g, 0.20 mmol) and Ruphos Pd G3 (0.08 g, 0.10 mmol) were added, and the reaction was heated at 100° C. for 16 hours. The reaction mixture was diluted with ethyl acetate (100 mL) and filtered through a pad of celite. The filtrate was collected and concentrated under vacuum to obtain crude product. The crude product was purified by flash chromatography (SiO₂, 230-400 mesh; 26% ethyl acetate in petroleum ether) to obtain tert-butyl(S)-4-((1-(3-(2,6-bis(benzyloxy)pyridin-3-yl)-1-methyl-1H-indazol-7-yl)pyrrolidin-3-yl)methyl)piperazine-1-carboxylate (0.39 g) as pale brown semi-solid.

LC-MS (ESI): m/z=689.7 [M+H]⁺.

### Preparation of tert-butyl 4-(((3S)-1-(3-(2,6-dioxopiperidin-3-yl)-1-methyl-1H-indazol-7-yl)pyrrolidin-3-yl)methyl)piperazine-1-carboxylate

To a stirred solution of tert-butyl(S)-4-((1-(3-(2,6-bis(benzyloxy)pyridin-3-yl)-1-methyl-1H-indazol-7-yl)pyrrolidin-3-yl)methyl)piperazine-1-carboxylate (0.38 g, 0.56 mmol), DMF (1.16 mL), and acetic acid (0.58 mL) in THF (6 mL), 10% Pd(OH)₂/C (1.18 g, 8.42 mmol) was added. The reaction was put under a hydrogen atmosphere (15 psi) and stirred at room temperature for 16 hours. The reaction mixture was diluted with THF:DCM (30%) and filtered through a pad of celite. The filtrate was concentrated under vacuum to afford tert-butyl 4-(((3S)-1-(3-(2,6-dioxopiperidin-3-yl)-1-methyl-1H-indazol-7-yl)pyrrolidin-3-yl)methyl)piperazine-1-carboxylate (0.37 g) as a pale brown semi-solid.

LC-MS (ESI): m/z=511.6 [M+H]⁺.

### Preparation of 3-(1-methyl-7-((S)-3-(piperazin-1-ylmethyl)pyrrolidin-1-yl)-1H-indazol-3-yl)piperidine-2,6-dione

To a stirred solution of tert-butyl 4-(((3S)-1-(3-(2,6-dioxopiperidin-3-yl)-1-methyl-1H-indazol-7-yl)pyrrolidin-3-yl)methyl)piperazine-1-carboxylate (0.36 g, 0.71 mmol) in DCM (5 mL), TFA (2.6 mL) was added at 0° C. The reaction was allowed to warm to room temperature over 2 hours with stirring. The reaction mixture was concentrated under vacuum to afford 3-(1-methyl-7-((S)-3-(piperazin-1-ylmethyl)pyrrolidin-1-yl)-1H-indazol-3-yl)piperidine-2,6-dione (0.31 g) as the TFA salt.

LC-MS (ESI): m/z=411.5 [M+H]⁺.

### Preparation of 2-((6-((5-chloro-2-(4-(((3S)-1-(3-(2,6-dioxopiperidin-3-yl)-1-methyl-1H-indazol-7-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide

To a stirred solution of 3-(1-methyl-7-((S)-3-(piperazin-1-ylmethyl)pyrrolidin-1-yl)-1H-indazol-3-yl)piperidine-2,6-dione. TFA salt (0.30 g, 0.73 mmol) in DMSO (6 mL), DIPEA (1.91 mL, 10.96 mmol) and 2-((6-((2,5-dichloropyrimidin-4-yl)amino)-1-methyl-2-oxo-1,2-dihydroquinolin-3-yl)oxy)-N-methylacetamide (0.19 g, 0.47 mmol) were added. The reaction was heated at 100° C. for 6 hours. The reaction mixture was quenched with ice-cold water. The precipitated solid was filtered and dried to obtain crude product. The crude product was purified by preparative HPLC to afford 2-((6-((5-chloro-2-(4-(((3S)-1-(3-(2,6-dioxopiperidin-3-yl)-1-methyl-1H-indazol-7-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (10.7 mg) as an off-white solid.

LC-MS (ESI): m/z=782.51 [M+H]⁺.

¹H NMR (400 MHZ, DMSO-d₆): δ 10.88 (s, 1H), 8.86 (s, 1H), 8.05 (s, 1H), 7.95 (s, 1H), 7.93 (d, J=2.00 Hz, 1H), 7.74 (dd, J=2.40, 9.20 Hz, 1H), 7.47 (d, J=9.20 Hz, 1H), 7.34 (d, J=7.60 Hz, 1H), 7.12 (s, 1H), 7.05-6.98 (m, 2H), 4.58 (s, 2H), 4.35-4.31 (m, 1H), 4.19 (s, 1H), 3.67 (s, 3H), 3.66-3.64 (m, 4H), 3.11-3.05 (m, 5H), 2.62 (d, J=8.00 Hz, 6H), 2.42-2.32 (m, 6H), 2.19-2.14 (m, 2H), 1.71-1.63 (m, 1H), 1.24-1.23 (m, 1H), 1.18-1.16 (m, 1H).

### Example 51. Synthesis of 2-((6-((5-chloro-2-(4-(((3S)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (Compound 306b)

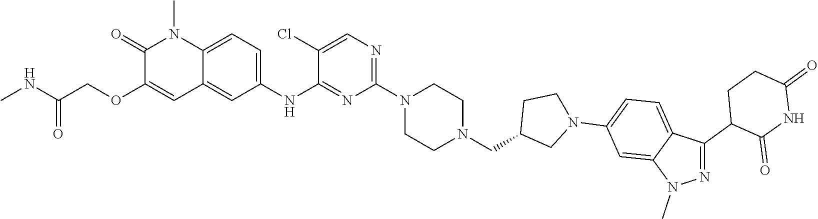 [SMILES: CNC(=O)COc1cc2cc(Nc3nc(N4CCN(C[C@@H]5CCN(c6ccc7c(C8CCC(=O)NC8=O)nn(C)c7c6)C5)CC4)ncc3Cl)ccc2n(C)c1=O]

### Preparation of 2-((6-((5-chloro-2-(4-(((3S)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide

2-((6-((5-chloro-2-(4-(((3S)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (0.033 g) was prepared as a pale brown solid in a similar manner as 2-((6-((5-chloro-2-(4-(((3R)-1-(3-(2,6-dioxopiperidin-3-yl)-1-methyl-1H-indazol-6-yl)pyrrolidin-3-yl)methyl)piperazin-1-yl)pyrimidin-4-yl)amino)-1-methyl-2-oxo-1,2-dihydroquinolin-3-yl)oxy)-N-methylacetamide (see, e.g., Example 49).

LC-MS (ESI): m/z=780.5 [M−H]⁻.

¹H NMR (400 MHZ, DMSO-d₆): δ 10.90 (s, 1H), 8.87 (s, 1H), 8.06 (s, 1H), 7.94-7.93 (m, 2H), 7.75-7.72 (m, 1H), 7.48-7.44 (m, 2H), 7.12 (s, 1H), 6.55 (d, J=8.80 Hz, 1H), 6.34 (s, 1H), 4.58 (s, 2H), 4.24-4.20 (m, 1H), 3.84 (s, 3H), 3.67 (s, 7H), 3.49-3.45 (m, 3H), 3.07 (t, J=16.00 Hz, 1H), 2.67-2.59 (m, 6H), 2.44-2.37 (m, 6H), 2.28-2.25 (m, 1H), 2.18-2.07 (m, 2H), 1.77-1.74 (m, 1H).

### Example 52. Synthesis of 2-((6-((5-chloro-2-((((3S)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (Compound 303a)

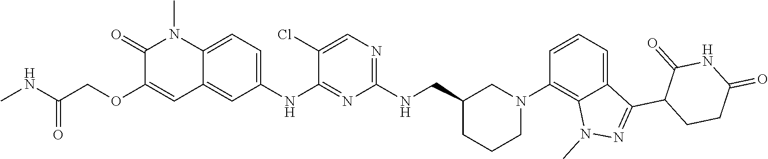 [SMILES: CNC(=O)COc1cc2cc(Nc3nc(NC[C@@H]4CCCN(c5cccc6c(C7CCC(=O)NC7=O)nn(C)c56)C4)ncc3Cl)ccc2n(C)c1=O]

### Preparation of tert-butyl(S)-((1-(3-(2,6-bis(benzyloxy)pyridin-3-yl)-1-methyl-1H-indazol-7-yl)piperidin-3-yl)methyl) carbamate

To a stirred suspension of 3-(2,6-bis(benzyloxy)pyridin-3-yl)-7-bromo-1-methyl-1H-indazole (0.7 g, 1.39 mmol) in 1,4-dioxane, tert-butyl (R)-(piperidin-3-ylmethyl) carbamate (0.45 g, 2.4 mmol) and cesium carbonate (1.36 g, 4.17 mmol) were added. The reaction was purged with argon for 15 minutes. Pd-PEPPSI-iHeptCl (0.07 g, 0.07 mmol) was added, and the reaction was heated at 100° C. for 3 hours. The reaction mixture was filtered through a celite bed and concentrated under vacuum to obtain crude product. The crude product was purified by column chromatography (SiO₂, 100-200 mesh; 30-50% EtOAc in petroleum ether) to afford tert-butyl (S)-((1-(3-(2,6-bis(benzyloxy)pyridin-3-yl)-1-methyl-1H-indazol-7-yl)piperidin-3-yl)methyl) carbamate (0.70 g) as an off-yellow solid.

LC-MS (ESI): m/z=634.7 [M+H]⁺.

### Preparation of tert-butyl (((3S)-1-(3-(2,6-dioxopiperidin-3-yl)-1-methyl-1H-indazol-7-yl)piperidin-3-yl)methyl) carbamate

A stirred solution of tert-butyl(S)-((1-(3-(2,6-bis(benzyloxy)pyridin-3-yl)-1-methyl-1H-indazol-7-yl)piperidin-3-yl)methyl) carbamate (0.70 g, 1.1 mmol) in THF (21 mL) was degassed with nitrogen. Palladium hydroxide on carbon (0.7 g) was added. The reaction was put under hydrogen atmosphere (15 psi) and stirred at room temperature for 4 hours. The reaction mixture was filtered through celite bed, and the celite was washed with THF. The filtrate was concentrated under reduced pressure to obtain crude product. The crude product was washed with n-pentane to afford tert-butyl (((3S)-1-(3-(2,6-dioxopiperidin-3-yl)-1-methyl-1H-indazol-7-yl)piperidin-3-yl)methyl) carbamate (0.30 g) as a brown solid. LC-MS (ESI): m/z=456.47 [M+H]⁺.

### Preparation of 3-(7-((S)-3-(aminomethyl)piperidin-1-yl)-1-methyl-1H-indazol-3-yl)piperidine-2,6-dione

A stirred solution of tert-butyl (((3S)-1-(3-(2,6-dioxopiperidin-3-yl)-1-methyl-1H-indazol-7-yl)piperidin-3-yl)methyl) carbamate (0.30 g, 0.66 mmol)) in DCM (6.0 mL) cooled to 0° C. TFA (3.0 mL) was added, and the reaction was allowed to warm to room temperature over 3 hours with stirring. The reaction mixture was concentrated under reduced pressure to obtain crude product. The crude product was triturated with n-pentane to afford 3-(7-((S)-3-(aminomethyl)piperidin-1-yl)-1-methyl-1H-indazol-3-yl)piperidine-2,6-dione (0.25 g) as a brown solid.

LC-MS (ESI): m/z=356.46 [M+H]⁺.

### Preparation of 2-((6-((5-chloro-2-((((3S)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide

To a stirred solution of 3-(7-((S)-3-(aminomethyl)piperidin-1-yl)-1-methyl-1H-indazol-3-yl)piperidine-2,6-dione (0.25 g, 0.56 mmol) and 2-((6-((2,5-dichloropyrimidin-4-yl)amino)-1-methyl-2-oxo-1,2-dihydroquinolin-3-yl)oxy)-N-methylacetamide (0.11 g, 0.28 mmol) in DMSO (5 mL), N,N-diisopropylethylamine (0.7 g, 5.6 mmol) was added. The reaction was stirred at 100° C. for 18 hours. The reaction mixture was poured into ice-cold water and stirred for 15 minutes. The resulting precipitate was filtered and dried to obtain crude compound. The crude product was purified by Prep-HPLC to afford 2-((6-((5-chloro-2-((((3S)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (0.05 g) as a brown solid.

LC-MS (ESI): m/z=727.49 [M+H]⁺.

¹H NMR (400 MHZ, DMSO-d₆): δ 10.86 (s, 1H), 8.74 (br s, 1H), 8.29 (s, 1H), 7.95-7.91 (m, 3H), 7.80 (br s, 1H), 7.36-7.33 (m, 2H), 7.20 (br s, 2H), 7.08-6.87 (m, 2H), 4.52 (s, 2H), 4.33-4.29 (m, 1H), 4.14-3.99 (m, 3H), 3.80-3.50 (br, 3H), 3.20-3.17 (br, 5H), 2.66-2.61 (m, 5H), 2.32-2.07 (m, 3H), 1.76-1.60 (m, 4H).

### Example 53. Synthesis of 2-((6-((5-chloro-2-((((3R)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (Compound 303b)

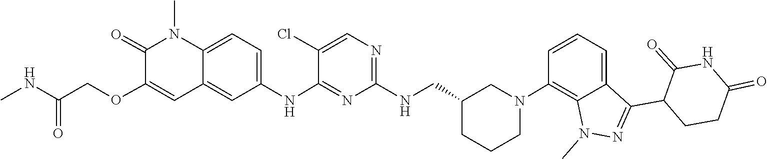 [SMILES: CNC(=O)COc1cc2cc(Nc3nc(NC[C@H]4CCCN(c5cccc6c(C7CCC(=O)NC7=O)nn(C)c56)C4)ncc3Cl)ccc2n(C)c1=O]

### Preparation of 2-((6-((5-chloro-2-((((3R)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide

2-((6-((5-chloro-2-((((3R)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (0.075 g) was prepared as an off-white solid in a similar manner as 2-((6-((5-chloro-2-((((3S)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (see, e.g., Example 52).

LC-MS (ESI): m/z=727.52 [M+H]⁺.

¹H NMR (400 MHZ, DMSO-d₆): δ 10.86 (s, 1H), 8.75 (s, 1H), 8.28 (s, 1H), 7.95-7.78 (m, 4H), 7.36-6.87 (m, 6H), 4.52 (s, 2H), 4.32-4.29 (m, 1H), 4.14-3.99 (m, 3H), 3.66-3.49 (m, 3H), 3.18-3.03 (m, 5H), 2.66-2.51 (m, 5H), 2.32-2.07 (m, 3H), 1.75-1.49 (m, 4H).

### Example 54. Synthesis of 2-((6-((5-chloro-2-((3R)-3-(((3-(2,6-dioxopiperidin-3-yl)-1-methyl-1H-indazol-6-yl)(methyl)amino)methyl)piperidin-1-yl)pyrimidin-4-yl)amino)-1-methyl-2-oxo-1,2-dihydroquinolin-3-yl)oxy)-N-methylacetamide (Compound 312a)

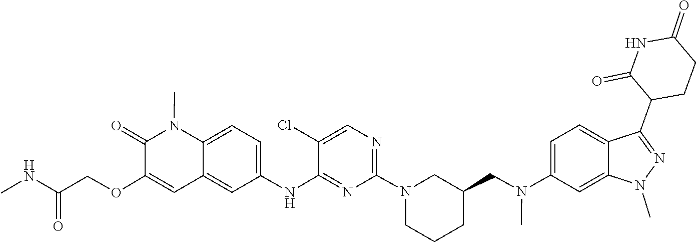 [SMILES: CNC(=O)COc1cc2cc(Nc3nc(N4CCC[C@H](CN(C)c5ccc6c(C7CCC(=O)NC7=O)nn(C)c6c5)C4)ncc3Cl)ccc2n(C)c1=O]

### Preparation of 2-((6-((5-chloro-2-((3R)-3-(((3-(2,6-dioxopiperidin-3-yl)-1-methyl-1H-indazol-6-yl)(methyl)amino)methyl)piperidin-1-yl)pyrimidin-4-yl)amino)-1-methyl-2-oxo-1,2-dihydroquinolin-3-yl)oxy)-N-methylacetamide

2-((6-((5-chloro-2-((3R)-3-(((3-(2,6-dioxopiperidin-3-yl)-1-methyl-1H-indazol-6-yl)(methyl)amino)methyl)piperidin-1-yl)pyrimidin-4-yl)amino)-1-methyl-2-oxo-1,2-dihydroquinolin-3-yl)oxy)-N-methylacetamide (0.025 g) was prepared as an off-white solid in a similar manner as 2-((6-((5-chloro-2-((3R)-3-(((3-(2,6-dioxopiperidin-3-yl)-1-methyl-1H-indazol-7-yl)(methyl)amino)methyl)piperidin-1-yl)pyrimidin-4-yl)amino)-1-methyl-2-oxo-1,2-dihydroquinolin-3-yl)oxy)-N-methylacetamide (see, e.g., Example 45).

LC-MS (ESI): m/z=741.49 [M+H]⁺.

¹H NMR (400 MHZ, DMSO-d₆): δ 10.9 (s, 1H), 8.79 (s, 1H), 8.02 (s, 1H), 7.93 (d, J=4.40 Hz, 1H), 7.85 (s, 1H), 7.77 (d, J=9.20 Hz, 1H), 7.34 (s, 2H), 7.13 (s, 1H), 6.60 (s, 1H), 6.43 (s, 1H), 4.52 (s, 1H), 4.42 (d, J=11.60 Hz, 1H), 4.32 (d, J=12.40 Hz, 1H), 4.22-4.18 (m, 1H), 3.78 (s, 3H), 3.57 (s, 1H), 3.26 (br, 2H), 2.96-2.86 (m, 5H), 2.66-2.64 (m, 5H), 2.27-2.23 (m, 1H) 2.17-2.12 (m, 1H), 1.89 (s, 1H), 1.70-1.67 (m, 4H), 1.29-1.24 (m, 3H).

### Example 55. Synthesis of 2-((6-((5-chloro-2-((3S)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (Compound 302a)

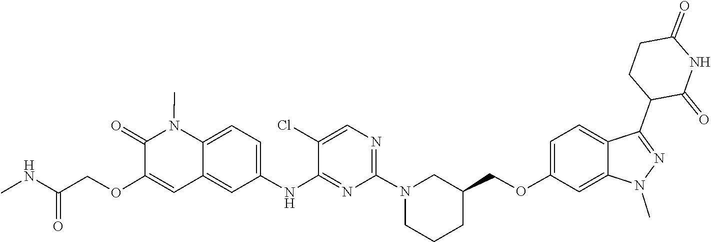 [SMILES: CNC(=O)COc1cc2cc(Nc3nc(N4CCC[C@H](COc5ccc6c(C7CCC(=O)NC7=O)nn(C)c6c5)C4)ncc3Cl)ccc2n(C)c1=O]

### Preparation of tert-butyl(S)-3-(((3-(2,6-bis(benzyloxy)pyridin-3-yl)-1-methyl-1H-indazol-6-yl)oxy)methyl)piperidine-1-carboxylate

To a stirred solution of tert-butyl(S)-3-(hydroxymethyl)piperidine-1-carboxylate (0.94 g, 4.36 mmol) and 3-(2,6-bis(benzyloxy)pyridin-3-yl)-6-bromo-1-methyl-1H-indazole (1.31 g, 2.61 mmol) in 1,4-dioxane (18.8 mL), potassium tert-butoxide (0.59 g, 5.23 mmol) was added. The reaction was degassed with nitrogen for 10 minutes. tBuBrettPhos Pd G3 (0.19 g, 0.21 mmol) was added to the reaction, and the reaction mixture was stirred to room temperature for 16 hours. The reaction was quenched with cold water and extracted into ethyl acetate. The organic layers were dried over anhydrous sodium sulfate, filtered, and concentrated to obtain crude product. The crude product was purified by flash column chromatography (SiO₂, 100-200, 15% ethyl acetate in petroleum ether) to afford tert-butyl(S)-3-(((3-(2,6-bis(benzyloxy)pyridin-3-yl)-1-methyl-1H-indazol-6-yl)oxy)methyl)piperidine-1-carboxylate (0.60 g) as a brown solid.

LC-MS (ESI): m/z=635.57 [M+H]⁺.

### Preparation of tert-butyl (3S)-3-(((3-(2,6-dioxopiperidin-3-yl)-1-methyl-1H-indazol-6-yl)oxy)methyl)piperidine-1-carboxylate

To a stirred solution of tert-butyl(S)-3-(((3-(2,6-bis(benzyloxy)pyridin-3-yl)-1-methyl-1H-indazol-6-yl)oxy)methyl)piperidine-1-carboxylate (0.6 g, 0.95 mmol) in THF (24 mL), palladium hydroxide on carbon, 10% Pd (0.6 g, w/w) was added. The reaction mixture was put under a hydrogen atmosphere (balloon pressure) and stirred at room temperature for 4 hours. The reaction mixture was filtered through a pad of celite, and the celite was washed with THF. The filtrate was concentrated to obtain crude product. The crude product was purified by flash column chromatography (SiO₂, 100-200, 30% ethyl acetate in petroleum ether) to afford tert-butyl (3S)-3-(((3-(2,6-dioxopiperidin-3-yl)-1-methyl-1H-indazol-6-yl)oxy)methyl)piperidine-1-carboxylate (0.15 g) as a yellow sticky solid.

LC-MS (ESI): m/z=457.51 [M+H]⁺.

### Preparation of 3-(1-methyl-6-(((S)-piperidin-3-yl) methoxy)-1H-indazol-3-yl)piperidine-2,6-dione

To a stirred solution of tert-butyl (3S)-3-(((3-(2,6-dioxopiperidin-3-yl)-1-methyl-1H-indazol-6-yl)oxy)methyl)piperidine-1-carboxylate (0.15 g, 0.33 mmol) in DCM (1.5 mL), 4 M HCl in 1,4-dioxane (0.75 mL) was added at 0° C. under a nitrogen atmosphere. The reaction was allowed to warm to room temperature over 5 hours with stirring. The reaction mixture was concentrated under reduced pressure to obtain crude product. The crude product was triturated with diethyl ether followed by n-pentane to afford 3-(1-methyl-6-(((S)-piperidin-3-yl) methoxy)-1H-indazol-3-yl)piperidine-2,6-dione hydrochloride (0.11 g) as a white solid.

LC-MS (ESI): m/z=357.42 [M+H]⁺.

### Preparation of 2-((6-((5-chloro-2-((3S)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(((S)-piperidin-3-yl) methoxy)-1H-indazol-3-yl)piperidine-2,6-dione hydrochloride (0.11 g, 0.27 mmol) in DMSO (3.3 mL), DIPEA (0.25 mL, 1.40 mmol) was added under a nitrogen atmosphere. The reaction was stirred for 10 minutes. 2-((6-((2,5-dichloropyrimidin-4-yl)amino)-1-methyl-2-oxo-1,2-dihydroquinolin-3-yl)oxy)-N-methylacetamide (0.08 g, 0.20 mmol) was added, and the reaction was heated to 120° C. for 5 hours. The reaction mixture was quenched with cold water. The resulting solid was filtered and washed with cold water and pet ether to obtain crude product. The crude product was purified by prep-HPLC to afford 2-((6-((5-chloro-2-((3S)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (0.030 g) as an off-white solid.

LC-MS (ESI): m/z=728.47 [M+H]⁺.

¹H NMR (400 MHZ, DMSO-d₆): δ 10.86 (s, 1H), 8.83 (s, 1H), 8.05 (s, 1H), 7.91 (s, 1H), 7.84 (s, 1H), 7.80-7.78 (d, J=8.4 Hz, 1H), 7.44-7.42 (d, J=9.2 Hz, 2H), 7.11 (s, 1H), 7.02 (s, 1H), 6.54 (br s, 1H), 4.59 (s, 1H), 4.46-4.27 (m, 4H), 3.95-3.89 (m, 5H), 3.60 (s, 3H), 3.00-2.85 (m, 2H), 2.67-2.62 (m, 5H), 2.33-2.29 (m, 1H), 2.19-2.15 (m, 1H), 1.99 (m, 1H), 1.91-1.89 (m, 1H), 1.75-1.72 (m, 1H), 1.49-1.37 (m, 2H).

### Example 56. Synthesis of 2-((6-((5-chloro-2-((3R)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (Compound 302b)

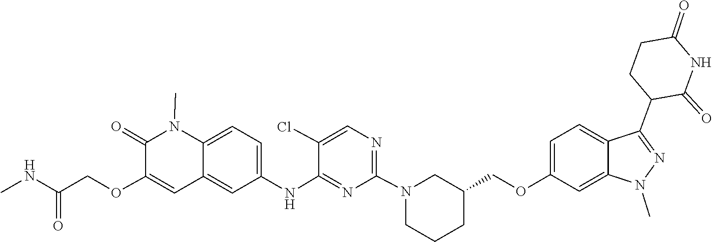 [SMILES: CNC(=O)COc1cc2cc(Nc3nc(N4CCC[C@@H](COc5ccc6c(C7CCC(=O)NC7=O)nn(C)c6c5)C4)ncc3Cl)ccc2n(C)c1=O]

### Preparation of 2-((6-((5-chloro-2-((3R)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide

2-((6-((5-chloro-2-((3R)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (0.013 g) was prepared as an off-white solid in a similar manner as 2-((6-((5-chloro-2-((3S)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (see, e.g., Example 55).

LC-MS (ESI): m/z=726.42 [M−H]⁻.

¹H NMR (400 MHz, DMSO-d₆): δ 10.86 (s, 1H), 8.84 (s, 1H), 8.05 (s, 1H), 7.91-7.80 (m, 3H), 7.43 (d, J=9.20 Hz, 2H), 7.12 (s, 1H), 7.02 (s, 1H), 6.60-6.54 (m, 1H), 4.46-4.28 (m, 5H), 4.00-3.80 (m, 5H), 3.61 (s, 3H), 2.97-2.86 (m, 2H), 2.63-2.64 (m, 5H), 2.33-2.31 (m, 1H), 2.19-2.17 (m, 1H), 1.98-1.89 (m, 1H), 1.75-1.72 (m, 1H), 1.41-1.43 (m, 1H), 1.49-1.46 (m, 2H).

### Example 57. Synthesis of 2-((6-((5-chloro-2-((((1r,4r)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide (Compound 293b)

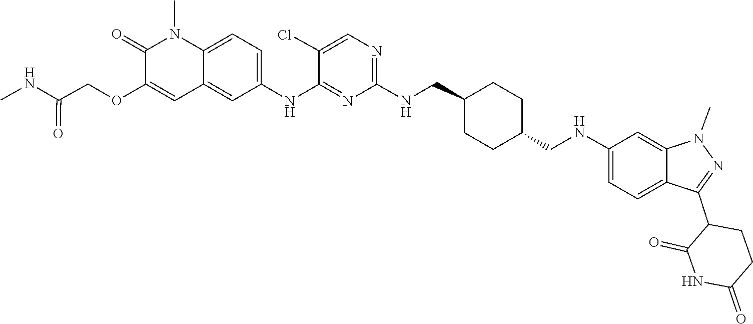 [SMILES: CNC(=O)COc1cc2cc(Nc3nc(NC[C@H]4CC[C@H](CNc5ccc6c(C7CCC(=O)NC7=O)nn(C)c6c5)CC4)ncc3Cl)ccc2n(C)c1=O]

### Preparation of 2-((6-((5-chloro-2-((((1r,4r)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide

2-((6-((5-chloro-2-((((1r,4r)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide (0.027 g) was prepared as an off-white solid in a similar manner as 2-((6-((5-chloro-2-((((1s,4s)-4-(((3-(2,6-dioxopiperidin-3-yl)-1-methyl-1H-indazol-6-yl)amino)methyl)cyclohexyl)methyl)amino)pyrimidin-4-yl)amino)-1-methyl-2-oxo-1,2-dihydroquinolin-3-yl)oxy)-N-methylacetamide (see, e.g., Example 38).

LC-MS (ESI): m/z=753.48 [M−H]⁻.

¹H NMR (400 MHZ, DMSO-d₆): δ 10.79 (s, 1H), 8.67 (bs, 1H), 8.32 (s, 1H), 8.02-7.94 (m, 3H), 7.83-7.81 (d, J=9.2 Hz, 1H), 7.44-7.42 (d, J=9.2 Hz, 1H), 7.3-7.09 (m, 3H), 6.54-6.52 (d, J=8.4 Hz, 1H), 6.27 (s, 1H), 5.87 (s, 1H), 4.55 (s, 2H), 4.18-4.14 (m, 1H), 3.79 (s, 3H), 3.66 (s, 3H), 3.04 (s, 2H), 2.89 (s, 2H), 2.67-2.66 (d, J=4.8 Hz, 3H), 2.59-2.54 (t, J=10.8 Hz, 2H), 2.32-2.12 (m, 2H), 1.85 (bs, 2H), 1.75 (s, 3H), 1.53 (bs, 2H), 1.11 (s, 1H), 0.99-0.89 (m, 4H).

### Example 58. Synthesis of 2-((6-((5-chloro-2-((3S)-3-(((3-(2,6-dioxopiperidin-3-yl)-1-methyl-1H-indazol-6-yl)oxy)methyl)pyrrolidin-1-yl)pyrimidin-4-yl)amino)-1-methyl-2-oxo-1,2-dihydroquinolin-3-yl)oxy)-N-methylacetamide (Compound 313a)

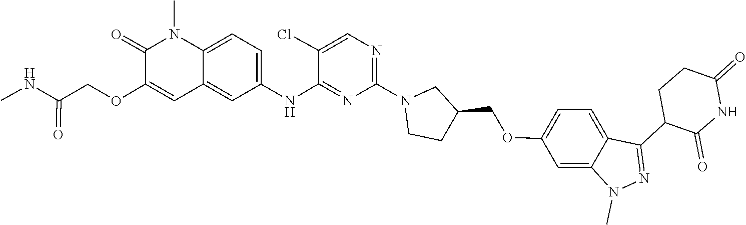 [SMILES: CNC(=O)COc1cc2cc(Nc3nc(N4CC[C@H](COc5ccc6c(C7CCC(=O)NC7=O)nn(C)c6c5)C4)ncc3Cl)ccc2n(C)c1=O]

### Preparation of 2-((6-((5-chloro-2-((3S)-3-(((3-(2,6-dioxopiperidin-3-yl)-1-methyl-1H-indazol-6-yl)oxy)methyl)pyrrolidin-1-yl)pyrimidin-4-yl)amino)-1-methyl-2-oxo-1,2-dihydroquinolin-3-yl)oxy)-N-methylacetamide

2-((6-((5-chloro-2-((3S)-3-(((3-(2,6-dioxopiperidin-3-yl)-1-methyl-1H-indazol-6-yl)oxy)methyl)pyrrolidin-1-yl)pyrimidin-4-yl)amino)-1-methyl-2-oxo-1,2-dihydroquinolin-3-yl)oxy)-N-methylacetamide (0.045 g) was prepared as a grey solid in a similar manner as 2-((6-((5-chloro-2-((3S)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (see, e.g., Example 55).

LC-MS (ESI): m/z=712.47 [M−H]⁻.

¹H NMR (400 MHZ, DMSO-d₆): δ 10.90 (s, 1H), 8.77 (s, 1H), 8.96 (d, J=2.0 Hz, 1H), 8.05 (s, 1H), 7.93 (br s, 1H), 7.88 (d, J=8.40 Hz, 1H), 7.55 (d, J=8.80 Hz, 1H), 7.46 (d, J=8.80 Hz, 1H), 7.15 (s, 1H) 7.10 (d, J=1.60 Hz, 1H), 6.75 (d, J=8.00 Hz, 1H), 4.31 (s, 2H), 4.29-4.27 (m, 1H), 4.10 (s, 2H), 3.91 (s, 3H), 3.67-3.63 (m, 4H), 3.50-3.49 (m, 2H), 2.80-2.79 (m, 1H), 2.64-2.61 (m, 4H), 2.32 (s, 1H), 2.18-2.17 (m, 2H), 1.90-1.89 (m, 1H), 1.26-1.24 (m, 2H).

### Example 59. Synthesis of 2-((6-((5-chloro-2-((4-(((3-(2,6-dioxopiperidin-3-yl)-1-methyl-1H-indazol-6-yl)amino)methyl)benzyl)amino)pyrimidin-4-yl)amino)-1-methyl-2-oxo-1,2-dihydroquinolin-3-yl)oxy)-N-methylacetamide (Compound 315)

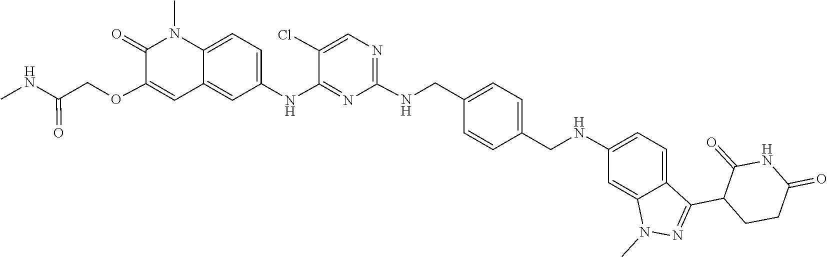 [SMILES: CNC(=O)COc1cc2cc(Nc3nc(NCc4ccc(CNc5ccc6c(C7CCC(=O)NC7=O)nn(C)c6c5)cc4)ncc3Cl)ccc2n(C)c1=O]

### Preparation of 2-((6-((5-chloro-2-((4-(((3-(2,6-dioxopiperidin-3-yl)-1-methyl-1H-indazol-6-yl)amino)methyl)benzyl)amino)pyrimidin-4-yl)amino)-1-methyl-2-oxo-1,2-dihydroquinolin-3-yl)oxy)-N-methylacetamide

2-((6-((5-chloro-2-((4-(((3-(2,6-dioxopiperidin-3-yl)-1-methyl-1H-indazol-6-yl)amino)methyl)benzyl)amino)pyrimidin-4-yl)amino)-1-methyl-2-oxo-1,2-dihydroquinolin-3-yl)oxy)-N-methylacetamide (0.043 g) was prepared as an off-white solid in a similar manner as 2-((6-((5-chloro-2-((((1r,4r)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide (see, e.g., Example 31).

LC-MS (ESI): m/z=747.51 [M−H]⁻.

¹H NMR (400 MHZ, DMSO-d₆): δ 10.79 (s, 1H), 8.68 (s, 1H), 8.22 (s, 2H), 7.97-7.92 (m, 2H), 7.74-6.62 (m, 8H), 6.56 (d, J=8.00 Hz, 1H), 6.47 (t, J=5.60 Hz, 1H), 6.30 (br s, 1H), 4.51 (s, 2H), 4.39 (s, 2H), 4.29 (d, J=5.20 Hz, 2H), 4.16-4.13 (m, 1H), 3.60 (br, 6H), 2.66 (d, J=4.80 Hz, 3H), 2.57-2.55 (m, 2H), 2.20-2.07 (m, 2H).

### Example 60. Synthesis of 2-((6-((5-chloro-2-((((1s,4s)-4-((3-(2,6-dioxopiperidin-3-yl)-1-methyl-1H-indazol-7-yl)amino)cyclohexyl)methyl)amino)pyrimidin-4-yl)amino)-1-methyl-2-oxo-1,2-dihydroquinolin-3-yl)oxy)-N-methylacetamide (Compound 304a)

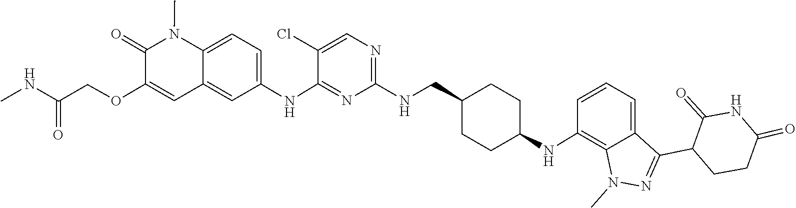 [SMILES: CNC(=O)COc1cc2cc(Nc3nc(NC[C@H]4CC[C@@H](Nc5cccc6c(C7CCC(=O)NC7=O)nn(C)c56)CC4)ncc3Cl)ccc2n(C)c1=O]

### Preparation of 2-((6-((5-chloro-2-((((1s,4s)-4-((3-(2,6-dioxopiperidin-3-yl)-1-methyl-1H-indazol-7-yl)amino)cyclohexyl)methyl)amino)pyrimidin-4-yl)amino)-1-methyl-2-oxo-1,2-dihydroquinolin-3-yl)oxy)-N-methylacetamide

2-((6-((5-chloro-2-((((1s,4s)-4-((3-(2,6-dioxopiperidin-3-yl)-1-methyl-1H-indazol-7-yl)amino)cyclohexyl)methyl)amino)pyrimidin-4-yl)amino)-1-methyl-2-oxo-1,2-dihydroquinolin-3-yl)oxy)-N-methylacetamide (0.011 g) was prepared as a white solid in a similar manner as 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (see, e.g., Example 44).

LC-MS (ESI): m/z=739.46 [M−H]⁻.

¹H NMR (400 MHZ, DMSO-d₆): δ 10.76 (s, 1H), 8.48 (s, 1H), 8.28 (s, 1H), 7.91 (s, 1H), 7.64 (s, 1H), 7.56 (s, 1H), 7.01-6.87 (m, 3H), 6.41-6.33 (m, 3H), 5.47 (s, 1H), 3.65-3.63 (m, 1H), 3.51 (s, 2H), 3.34 (s, 2H), 3.22 (s, 3H), 3.001 (s, 2H), 2.07-2.009 (m, 3H), 1.76-1.73 (t, J=13.6 Hz, 2H).

### Example 61. Synthesis of 2-((6-((5-chloro-2-((((1r,4r)-4-((3-(2,6-dioxopiperidin-3-yl)-1-methyl-1H-indazol-7-yl)amino)cyclohexyl)methyl)amino)pyrimidin-4-yl)amino)-1-methyl-2-oxo-1,2-dihydroquinolin-3-yl)oxy)-N-methylacetamide (Compound 304b)

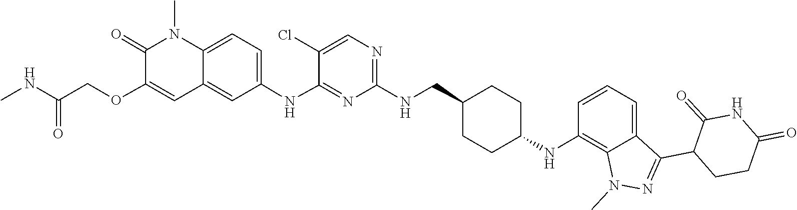 [SMILES: CNC(=O)COc1cc2cc(Nc3nc(NC[C@H]4CC[C@H](Nc5cccc6c(C7CCC(=O)NC7=O)nn(C)c56)CC4)ncc3Cl)ccc2n(C)c1=O]

### Preparation of 2-((6-((5-chloro-2-((((1r,4r)-4-((3-(2,6-dioxopiperidin-3-yl)-1-methyl-1H-indazol-7-yl)amino)cyclohexyl)methyl)amino)pyrimidin-4-yl)amino)-1-methyl-2-oxo-1,2-dihydroquinolin-3-yl)oxy)-N-methylacetamide

2-((6-((5-chloro-2-((((1r,4r)-4-((3-(2,6-dioxopiperidin-3-yl)-1-methyl-1H-indazol-7-yl)amino)cyclohexyl)methyl)amino)pyrimidin-4-yl)amino)-1-methyl-2-oxo-1,2-dihydroquinolin-3-yl)oxy)-N-methylacetamide (0.006 g) was prepared as an off-white solid in a similar manner as 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (see, e.g., Example 44).

LC-MS (ESI): m/z=741.40 [M+H]⁺.

¹H NMR (400 MHZ, DMSO-d₆): δ 10.84 (s, 1H), 8.70-8.48 (m, 4H), 8.04-7.83 (m, 4H), 7.45-7.43 (d, J=9.2 Hz, 1H), 7.19-6.84 (m, 4H), 6.47 (bs, 1H), 4.84 (s, 1H), 4.56 (s, 2H), 4.26-4.20 (m, 4H), 3.67 (s, 3H), 3.10 (s, 3H), 2.66-2.60 (m, 5H), 2.28-2.26 (m, 1H), 2.15-2.06 (m, 4H), 1.76 (br s, 2H), 1.58 (m, 2H), 1.23 (s, 5H), 1.05 (m, 1H).

### Example 62. Synthesis of 2-((6-((5-chloro-2-(4-((1-(3-(2,6-dioxopiperidin-3-yl)-1-methyl-1H-indazol-7-yl)piperidin-4-yl)oxy)piperidin-1-yl)pyrimidin-4-yl)amino)-1-methyl-2-oxo-1,2-dihydroquinolin-3-yl)oxy)-N-methylacetamide (Compound 316)

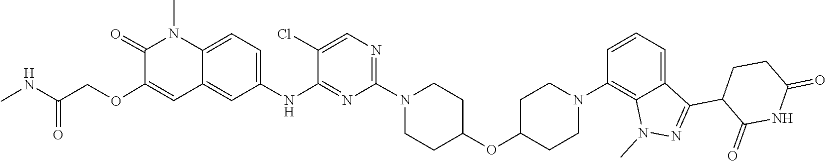 [SMILES: CNC(=O)COc1cc2cc(Nc3nc(N4CCC(OC5CCN(c6cccc7c(C8CCC(=O)NC8=O)nn(C)c67)CC5)CC4)ncc3Cl)ccc2n(C)c1=O]

### Preparation of 2-((6-((5-chloro-2-(4-((1-(3-(2,6-dioxopiperidin-3-yl)-1-methyl-1H-indazol-7-yl)piperidin-4-yl)oxy)piperidin-1-yl)pyrimidin-4-yl)amino)-1-methyl-2-oxo-1,2-dihydroquinolin-3-yl)oxy)-N-methylacetamide

2-((6-((5-chloro-2-(4-((1-(3-(2,6-dioxopiperidin-3-yl)-1-methyl-1H-indazol-7-yl)piperidin-4-yl)oxy)piperidin-1-yl)pyrimidin-4-yl)amino)-1-methyl-2-oxo-1,2-dihydroquinolin-3-yl)oxy)-N-methylacetamide (0.115 g) was prepared as an off-white solid in a similar manner as 2-((6-((5-chloro-2-(4-((1-(3-(2,6-dioxopiperidin-3-yl)-1-methyl-1H-indazol-7-yl)piperidin-4-yl)methyl)piperidin-1-yl)pyrimidin-4-yl)amino)-1-methyl-2-oxo-1,2-dihydroquinolin-3-yl)oxy)-N-methylacetamide (see, e.g., Example 21).

LC-MS (ESI): m/z=795.55 [M−H]⁻.

¹H NMR (400 MHZ, DMSO-d₆): δ 10.89 (s, 1H), 8.84 (s, 1H), 8.05 (s, 1H), 7.96-7.93 (m, 2H), 7.74 (dd, J=2.40 Hz, 9.00 Hz, 1H), 7.48 (d, J=9.2 Hz, 1H), 7.37-7.35 (m, 1H), 7.10 (s, 1H), 7.01-6.98 (m, 2H), 4.59 (s, 2H), 4.34-4.31 (m, 1H), 4.24 (s, 3H), 4.10-4.07 (m, 2H), 3.75-3.67 (m, 5H), 3.23 (br s, 4H), 2.74-2.57 (m, 7H), 2.34-2.28 (m, 1H), 2.18-2.07 (m, 1H), 2.25-1.87 (m, 1H), 1.86-1.88-1.84 (m, 3H), 1.69-1.66 (br s, 2H), 1.48-1.43 (m, 2H).

### Example 63. Synthesis of 2-((6-((5-chloro-2-((2S)-2-(((3-(2,6-dioxopiperidin-3-yl)-1-methyl-1H-indazol-6-yl)oxy)methyl) morpholino)pyrimidin-4-yl)amino)-1-methyl-2-oxo-1,2-dihydroquinolin-3-yl)oxy)-N-methylacetamide (Compound 317a)

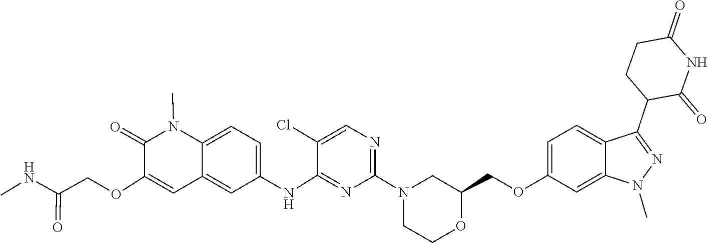 [SMILES: CNC(=O)COc1cc2cc(Nc3nc(N4CCO[C@H](COc5ccc6c(C7CCC(=O)NC7=O)nn(C)c6c5)C4)ncc3Cl)ccc2n(C)c1=O]

### Preparation of 2-((6-((5-chloro-2-((2S)-2-(((3-(2,6-dioxopiperidin-3-yl)-1-methyl-1H-indazol-6-yl)oxy)methyl) morpholino)pyrimidin-4-yl)amino)-1-methyl-2-oxo-1,2-dihydroquinolin-3-yl)oxy)-N-methylacetamide

2-((6-((5-chloro-2-((2S)-2-(((3-(2,6-dioxopiperidin-3-yl)-1-methyl-1H-indazol-6-yl)oxy)methyl) morpholino)pyrimidin-4-yl)amino)-1-methyl-2-oxo-1,2-dihydroquinolin-3-yl)oxy)-N-methylacetamide (0.003 g) was prepared as an off-white solid in a similar manner as 2-((6-((5-chloro-2-((3S)-3-(((3-(2,6-dioxopiperidin-3-yl)-1-methyl-1H-indazol-6-yl)oxy)methyl)piperidin-1-yl)pyrimidin-4-yl)amino)-1-methyl-2-oxo-1,2-dihydroquinolin-3-yl)oxy)-N-methylacetamide (see, e.g., Example 55).

LC-MS (ESI): m/z=730.45 [M+H]⁺.

¹H NMR (400 MHZ, DMSO-d₆): δ 10.9 (s, 1H), 8.94 (s, 1H), 8.45 (s, 2H), 8.10 (s, 1H), 7.89 (m, 2H), 7.76-7.75 (d, 1H J-4), 7.53-7.51 (d, 1H, J=8), 7.46-7.44 (d, 1H, J=8), 7.16 (s, 1H), 7.09 (s, 1H), 6.67-6.66 (d, 1H, J-4), 4.49 (s, 2H), 4.31-4.28 (m, 2H), 4.13-4.12 (d, 1H, J=4), 3.98-3.35 (d, 1H, J=12), 3.84 (s, 3H), 3.60-3.54 (m, 1H), 3.50 (s, 4H), 3.04-2.98 (t, 1H, J=24), 2.91-2.85 (t, 1H, J=24), 2.67-2.63 (m, 5H), 2.37-2.34 (m, 1H), 2.23-2.18 (m, 1H), 1.23 (s, 2H).

### Example 64. Synthesis of 2-((6-((5-chloro-2-((((1r,4r)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (Compound 297b)

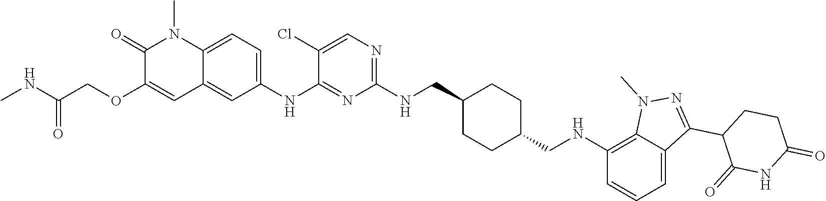 [SMILES: CNC(=O)COc1cc2cc(Nc3nc(NC[C@H]4CC[C@H](CNc5cccc6c(C7CCC(=O)NC7=O)nn(C)c56)CC4)ncc3Cl)ccc2n(C)c1=O]

### Preparation of 2-((6-((5-chloro-2-((((1r,4r)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide

2-((6-((5-chloro-2-((((1r,4r)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (0.035 g) was prepared as a brown solid in a similar manner as 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (see, e.g., Example 44).

LC-MS (ESI): m/z=753.52 [M−H]⁻.

¹H NMR (400 MHZ, DMSO-d₆): δ 10.79 (s, 1H), 8.68 (bs, 1H), 8.43 (s, 1H), 8.03-7.94 (m, 3H), 7.83-7.81 (m, 1H), 7.44-7.42 (d, J=9.2 Hz, 1H), 7.1-6.8 (m, 4H), 6.40-6.38 (d, J=7.2 Hz, 1H), 5.32 (s, 1H), 4.55 (s, 2H), 4.26-4.22 (m, 4H), 3.32 (s, 3H), 2.65-2.45 (m, 4H), 2.30-2.27 (m, 6H), 2.25-2.11 (m, 2H), 1.89 (bs, 2H), 1.76 (bs, 2H), 1.63 (bs, 1H), 1.52 (bs, 1H), 0.95-0.91 (m, 5H).

### Example 65. Synthesis of 2-((6-((5-chloro-2-((4-(((3-(2,6-dioxopiperidin-3-yl)-1-methyl-1H-indazol-7-yl)amino)methyl)benzyl)amino)pyrimidin-4-yl)amino)-1-methyl-2-oxo-1,2-dihydroquinolin-3-yl)oxy)-N-methylacetamide (Compound 318)

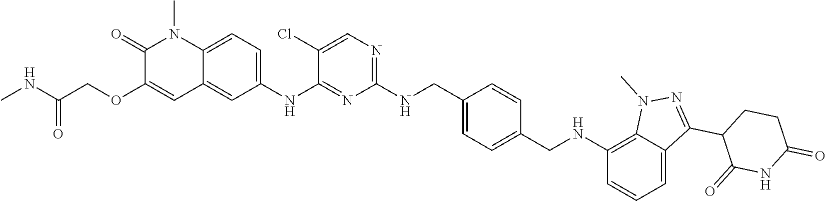 [SMILES: CNC(=O)COc1cc2cc(Nc3nc(NCc4ccc(CNc5cccc6c(C7CCC(=O)NC7=O)nn(C)c56)cc4)ncc3Cl)ccc2n(C)c1=O]

### Preparation of 2-((6-((5-chloro-2-((4-(((3-(2,6-dioxopiperidin-3-yl)-1-methyl-1H-indazol-7-yl)amino)methyl)benzyl)amino)pyrimidin-4-yl)amino)-1-methyl-2-oxo-1,2-dihydroquinolin-3-yl)oxy)-N-methylacetamide

2-((6-((5-chloro-2-((4-(((3-(2,6-dioxopiperidin-3-yl)-1-methyl-1H-indazol-7-yl)amino)methyl)benzyl)amino)pyrimidin-4-yl)amino)-1-methyl-2-oxo-1,2-dihydroquinolin-3-yl)oxy)-N-methylacetamide (0.017 g) was prepared as a pale brown solid in a similar manner as 2-((6-((5-chloro-2-((((1r,4r)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide (see, e.g., Example 31).

LC-MS (ESI): m/z=747.34 [M−H];

¹H NMR (400 MHZ, DMSO-d₆): δ 10.84 (s, 1H), 8.68 (s, 1H), 7.97-792 (m, 2H), 7.80-7.05 (m, 7H), 7.03-6.55 (m, 2H), 6.26 (br s, 1H), 6.05-6.03 (m, 1H), 4.51-4.22 (m, 10H), 3.62 (br s, 3H), 2.67-2.51 (m, 5H), 2.33-2.12 (m, 2H), 2.14-2.12 (m, 2H).

### Example 66. Synthesis of 2-((6-((5-chloro-2-((3S)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide (Compound 301a)

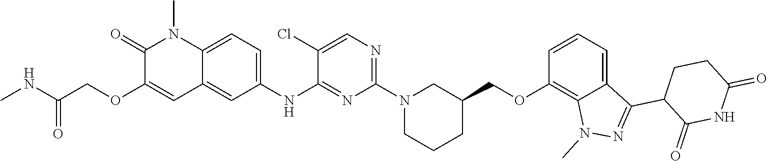 [SMILES: CNC(=O)COc1cc2cc(Nc3nc(N4CCC[C@H](COc5cccc6c(C7CCC(=O)NC7=O)nn(C)c56)C4)ncc3Cl)ccc2n(C)c1=O]

### Preparation of 3-(2,6-bis(benzyloxy)pyridin-3-yl)-1-methyl-1H-indazol-7-ol

To a stirred solution of 3-(2,6-bis(benzyloxy)pyridin-3-yl)-7-bromo-1-methyl-1H-indazole (1.0 g, 1.99 mmol) in dioxane:water (25 mL: 5 mL), potassium hydroxide (0.78 g, 13.98 mmol) was added. The reaction was degassed with argon for 5 minutes. Tert-Butyl xphos (0.17 g, 0.40 mmol) and Pd₂(dba)₃ (0.18 g, 0.20 mmol) were added, and the reaction was purged with argon for 5 minutes. The reaction was heated at 100° C. for 18 hours. The reaction mixture was diluted with ethyl acetate (100 mL) and filtered through a pad of celite. The filtrate was concentrated under vacuum to obtain crude product. The crude product was purified by flash chromatography (230-400 silica gel; 30-11% EtOAc in petroleum ether) to afford 3-(2,6-bis(benzyloxy)pyridin-3-yl)-1-methyl-1H-indazol-7-ol (0.37 g) as a pale green semi-solid.

LC-MS (ESI): m/z=438.43 [M+H]⁺.

### Preparation of tert-butyl(S)-3-(((methylsulfonyl)oxy)methyl)piperidine-1-carboxylate

To a stirred solution of tert-butyl(S)-3-(hydroxymethyl)piperidine-1-carboxylate (1.0 g, 4.64 mmol) in DCM (20 mL), TEA (1.91 mL, 13.93 mmol) was added. After the reaction was stirred for 10 minutes, the reaction was cooled to 0° C. and put under a nitrogen atmosphere. Methanesulfonyl chloride (0.4 mL, 5.57 mmol) was added, and the reaction was allowed to warm to room temperature over 2 hours with stirring. The reaction mixture was quenched with saturated ammonium bicarbonate solution (20 mL) and diluted with DCM (100 mL). The separated organic layers were dried over anhydrous Na₂SO₄, filtered, and dried under vacuum to afford tert-butyl(S)-3-(((methylsulfonyl)oxy)methyl)piperidine-1-carboxylate (0.54 g) as a pale yellow semi-solid.

LC-MS (ESI): m/z=296.32 [M+H]⁺.

### Preparation of tert-butyl(S)-3-(((3-(2,6-bis(benzyloxy)pyridin-3-yl)-1-methyl-1H-indazol-7-yl)oxy)methyl)piperidine-1-carboxylate

To a stirred solution of 3-(2,6-bis(benzyloxy)pyridin-3-yl)-1-methyl-1H-indazol-7-ol (0.50 g, 1.14 mmol) in DMF (5 mL), tert-butyl(S)-3-(((methylsulfonyl)oxy)methyl)piperidine-1-carboxylate (0.67 g, 2.29 mmol) and cesium carbonate (1.12 g, 3.43 mmol) were added. The reaction was heated at 100° C. for 18 hours. The reaction mixture was quenched with ice-cold water (100 mL) and extracted into ethyl acetate (200 mL×3). The separated organic layers were dried over anhydrous Na₂SO₄, filtered, and dried to obtain tert-butyl(S)-3-(((3-(2,6-bis(benzyloxy)pyridin-3-yl)-1-methyl-1H-indazol-7-yl)oxy)methyl)piperidine-1-carboxylate (0.33 g) as a pale green semi-solid. LC-MS (ESI): m/z=635.66 [M+H]⁺.

### Preparation of tert-butyl (3S)-3-(((3-(2,6-dioxopiperidin-3-yl)-1-methyl-1H-indazol-7-yl)oxy)methyl)piperidine-1-carboxylate

To a stirred solution of tert-butyl(S)-3-(((3-(2,6-bis(benzyloxy)pyridin-3-yl)-1-methyl-1H-indazol-7-yl)oxy)methyl)piperidine-1-carboxylate (0.28 g, 0.44 mmol) in THF (5.7 mL), DMF (0.9 mL) and AcOH (0.03 mL, 0.44 mmol) were added. 20% Pd(OH)₂/C (0.25 g, 1.76 mmol) was then added to the reaction. The reaction was put under a hydrogen atmosphere (15 psi) and stirred at room temperature for 18 hours. The reaction mixture was diluted with DCM (200 mL) and filtered through a pad of celite. The filtrate was concentrated under vacuum to obtain tert-butyl (3S)-3-(((3-(2,6-dioxopiperidin-3-yl)-1-methyl-1H-indazol-7-yl)oxy)methyl)piperidine-1-carboxylate (0.20 g) as a pale brown semi-solid.

LC-MS (ESI): m/z=457.24 [M+H]⁺.

### Preparation of 3-(1-methyl-7-(((S)-piperidin-3-yl) methoxy)-1H-indazol-3-yl)piperidine-2,6-dione

To a stirred solution of tert-butyl (3S)-3-(((3-(2,6-dioxopiperidin-3-yl)-1-methyl-1H-indazol-7-yl)oxy)methyl)piperidine-1-carboxylate (0.20 g, 0.43 mmol) in DCM (2.0 mL), TFA (1.60 mL) at 0° C. was added. The reaction was allowed to warm to room temperature over 1 hour. The reaction mixture was concentrated under vacuum to obtain 3-(1-methyl-7-(((S)-piperidin-3-yl) methoxy)-1H-indazol-3-yl)piperidine-2,6-dione (TFA salt)(0.33 g) as a pale brown semi-solid.

LC-MS (ESI): m/z=357.39 [M+H]⁺.

### Preparation of 2-((6-((5-chloro-2-((3S)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-7-(((S)-piperidin-3-yl) methoxy)-1H-indazol-3-yl)piperidine-2,6-dione (0.17 g, 0.47 mmol) in DMSO (2.55 mL), 2-((6-((2,5-dichloropyrimidin-4-yl)amino)-1-methyl-2-oxo-1,2-dihydroquinolin-3-yl)oxy)-N-methylacetamide (0.09 g, 0.23 mmol) and DIPEA (0.83 mL, 4.77 mmol) were added. The reaction was stirred at 100° C. for 6 hours. The reaction mixture was quenched with ice-cold water (20 mL). The precipitate was filtered and dried under vacuum to obtain crude product. The crude product was purified by preparative HPLC to afford 2-((6-((5-chloro-2-((3S)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide (0.075 g) as an off-white solid.

LC-MS (ESI): m/z=728.45 [M+H]⁺.

¹H NMR (400 MHZ, DMSO-d₆): 10.86 (s, 1H), 8.86 (s, 1H), 8.05 (s, 1H), 7.92-7.88 (m, 2H), 7.78-7.75 (m, 1H), 7.37 (br s, 1H), 7.23-7.21 (m, 1H), 7.09 (d, J=7.60 Hz, 1H), 6.98-6.94 (m, 1H), 6.80 (d, J=7.60 Hz, 1H), 4.68-4.47 (m, 1H), 4.41-4.29 (m, 4H), 4.02 (d, J=5.60 Hz, 4H), 3.53-3.51 (m, 3H), 3.01-2.91 (m, 2H), 2.66 (d, J=4.80 Hz, 6H), 2.39-2.34 (m, 1H), 2.21-2.18 (m, 1H), 2.08-2.01 (m, 1H), 1.95-1.94 (m, 1H), 1.76-1.75 (m, 1H), 1.48-1.46 (m, 2H).

### Example 67. Synthesis of 2-((6-((5-chloro-2-((3R)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide (Compound 301b)

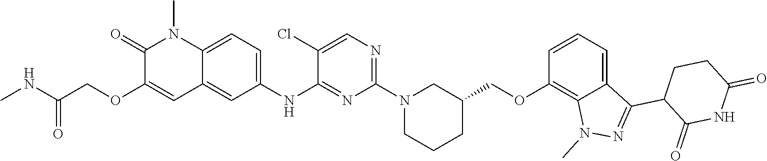 [SMILES: CNC(=O)COc1cc2cc(Nc3nc(N4CCC[C@@H](COc5cccc6c(C7CCC(=O)NC7=O)nn(C)c56)C4)ncc3Cl)ccc2n(C)c1=O]

### Preparation of 2-((6-((5-chloro-2-((3R)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide

2-((6-((5-chloro-2-((3R)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide (0.095 g) was prepared as an off-white solid in a similar manner as 2-((6-((5-chloro-2-((3S)-3-(((3-(2,6-dioxopiperidin-3-yl)-1-methyl-1H-indazol-7-yl)oxy)methyl)piperidin-1-yl)pyrimidin-4-yl)amino)-1-methyl-2-oxo-1,2-dihydroquinolin-3-yl)oxy)-N-methylacetamide (see, e.g., Example 66).

LC-MS (ESI): m/z=728.45 [M+H]⁺.

¹H NMR (400 MHz, DMSO-d₆): δ 10.87 (s, 1H), 8.85 (s, 1H), 8.05 (s, 1H), 7.91-7.87 (m, 2H), 7.78-7.74 (m, 1H), 7.37 (br s, 1H), 7.21 (dd, J=2.80, 8.20 Hz, 1H), 7.09 (d, J=7.60 Hz, 1H), 6.97-6.93 (m, 1H), 6.79 (d, J=7.20 Hz, 1H), 4.71-4.61 (m, 1H), 4.47-4.30 (m, 4H), 4.28-4.01 (m, 4H), 3.81-3.78 (m, 1H), 3.51-3.48 (m, 2H), 2.96-2.91 (m, 2H), 2.66 (d, J=4.80 Hz, 5H), 2.32-2.31 (m, 1H), 2.21-2.19 (m, 1H), 2.07-2.06 (m, 2H), 1.77-1.74 (m, 2H), 1.50 (d, J=9.20 Hz, 2H).

### Example 68. Synthesis of 2-((6-((5-chloro-2-((3R)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (Compound 308a)

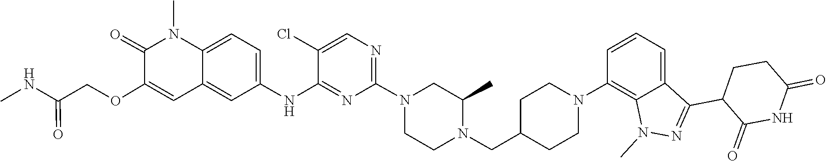 [SMILES: CNC(=O)COc1cc2cc(Nc3nc(N4CCN(CC5CCN(c6cccc7c(C8CCC(=O)NC8=O)nn(C)c67)CC5)[C@H](C)C4)ncc3Cl)ccc2n(C)c1=O]

### Preparation of tert-butyl (R)-4-((1-((benzyloxy)carbonyl)piperidin-4-yl)methyl)-3-methylpiperazine-1-carboxylate

To a solution of benzyl piperazine-1-carboxylate (8.8 g, 36.0 mmol), tert-butyl 4-formylpiperidine-1-carboxylate (6.0 g, 30.0 mmol) and NaBH(OAc)₃ (9.4 g, 45.0 mmol) in DCM (120 mL) was added acetic acid (1.8 mL, 30.0 mmol). The reaction was stirred at room temperature for 10 hours. The reaction mixture was quenched with cold water (200 mL) and extracted into DCM (3×200 mL). The combined organic layers were washed with brine solution (200 mL), dried over anhydrous Na₂SO₄, filtered, and dried under vacuum to get crude product. The crude product was purified by column chromatography (SiO₂; 40% EtOAc in petroleum ether) to afford tert-butyl (R)-4-((1-((benzyloxy)carbonyl)piperidin-4-yl)methyl)-3-methylpiperazine-1-carboxylate (6.1 g) as a colorless liquid.

LC-MS (ESI): m/z=432.35 [M+H]⁺.

### Preparation of tert-butyl (R)-3-methyl-4-(piperidin-4-ylmethyl)piperazine-1-carboxylate

To a stirred solution of tert-butyl (R)-4-((1-((benzyloxy)carbonyl)piperidin-4-yl)methyl)-3-methylpiperazine-1-carboxylate (6.0 g, 13.9 mmol) in THF (180 mL) was added 20% Pd(OH)₂ (3.0 g, 50%, w/w). The reaction was put under hydrogen atmosphere (60 psi) and stirred at room temperature for 16 hours. The reaction mixture was filtered through a celite bed and concentrated to obtain crude product. The crude product was washed with n-pentane and dried to afford tert-butyl (R)-3-methyl-4-(piperidin-4-ylmethyl)piperazine-1-carboxylate (3.8 g) as a colorless liquid.

LC-MS (ESI): m/z=298.24 [M+H]⁺.

### Preparation of tert-butyl (R)-4-((1-(3-(2,6-bis(benzyloxy)pyridin-3-yl)-1-methyl-1H-indazol-7-yl)piperidin-4-yl)methyl)-3-methylpiperazine-1-carboxylate

To a stirred solution of 3-(2,6-bis(benzyloxy)pyridin-3-yl)-7-bromo-1-methyl-1H-indazole (1.2 g, 2.40 mmol) and tert-butyl (R)-3-methyl-4-(piperidin-4-ylmethyl)piperazine-1-carboxylate (0.85 g, 2.88 mmol) in 1,4-dioxane (24 mL) was added cesium carbonate (2.34 g, 7.20 mmol). The reaction was degassed with argon for 10 minutes, Pd-PEPPSI-iHeptCl (0.11 g, 0.12 mmol) was added, and the reaction was heated at 100° C. for 12 hours. The reaction mixture was filtered through a celite bed and concentrated to obtain crude product. The crude product was purified by flash column chromatography by using (230-400 mesh silica gel, 30% ethyl acetate in petroleum ether) to afford tert-butyl (R)-4-((1-(3-(2,6-bis(benzyloxy)pyridin-3-yl)-1-methyl-1H-indazol-7-yl)piperidin-4-yl)methyl)-3-methylpiperazine-1-carboxylate (1.0 g) as a pale yellow semisolid.

LC-MS (ESI): m/z=717.64 [M+H]⁺.

### Preparation of tert-butyl (3R)-4-((1-(3-(2,6-dioxopiperidin-3-yl)-1-methyl-1H-indazol-7-yl)piperidin-4-yl)methyl)-3-methylpiperazine-1-carboxylate

To a stirred solution of tert-butyl (R)-4-((1-(3-(2,6-bis(benzyloxy)pyridin-3-yl)-1-methyl-1H-indazol-7-yl)piperidin-4-yl)methyl)-3-methylpiperazine-1-carboxylate (1.0 g, 1.39 mmol) in THF (20 mL) was added 20% Pd(OH)₂ (100% w/w, 1.0 g). The reaction was put under hydrogen atmosphere (60 psi) and stirred at room temperature for 6 hours. The reaction mixture was filtered through a celite bed and concentrated to obtain tert-butyl (3R)-4-((1-(3-(2,6-dioxopiperidin-3-yl)-1-methyl-1H-indazol-7-yl)piperidin-4-yl)methyl)-3-methylpiperazine-1-carboxylate as (0.75 g) as a pale brown semi-solid.

LC-MS (ESI): m/z=539.37 [M+H]⁺.

### Preparation of 3-(1-methyl-7-(4-(((R)-2-methylpiperazin-1-yl)methyl)piperidin-1-yl)-1H-indazol-3-yl)piperidine-2,6-dione

To a stirred solution of tert-butyl (3R)-4-((1-(3-(2,6-dioxopiperidin-3-yl)-1-methyl-1H-indazol-7-yl)piperidin-4-yl)methyl)-3-methylpiperazine-1-carboxylate (0.70 g, 1.3 mmol) in DCM (14 mL) was added TFA (2.1 mL). The reaction was stirred at room temperature for 3 hours. The reaction mixture was concentrated to obtain crude product. The reaction mixture was concentrated and co-distilled with petroleum ether to obtain 3-(1-methyl-7-(4-(((R)-2-methylpiperazin-1-yl)methyl)piperidin-1-yl)-1H-indazol-3-yl)piperidine-2,6-dione (0.60 g) as a brown solid.

LC-MS (ESI): m/z=439.57 [M+H]⁺.

### Preparation of 2-((6-((5-chloro-2-((3R)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide

To a stirred solution 3-(1-methyl-7-(4-(((R)-2-methylpiperazin-1-yl)methyl)piperidin-1-yl)-1H-indazol-3-yl)piperidine-2,6-dione (0.30 g, 0.68 mmol) and 2-((6-((2,5-dichloropyrimidin-4-yl)amino)-1-methyl-2-oxo-1,2-dihydroquinolin-3-yl)oxy)-N-methylacetamide (0.11 g, 0.27 mmol) in DMSO (6 mL), DIPEA (1.1 mL, 6.8 mmol) was added. The reaction was stirred at 100° C. for 12 hours. The reaction was cooled to room temperature and the solvent was removed using a centrifugal evaporator to obtain crude product. The crude product was purified by prep HPLC to afford 2-((6-((5-chloro-2-((3R)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (70 mg) as an off-white solid.

LC-MS (ESI): m/z=808.50 [M−H]⁻.

¹H NMR (400 MHZ, DMSO-d₆): δ 10.87 (s, 1H), 8.86 (s, 1H), 8.05 (s, 1H), 7.95 (d, J=2.40 Hz, 2H), 7.74 (dd, J=2.00, 9.00 Hz, 1H), 7.48 (d, J=9.20 Hz, 1H), 7.37-7.35 (m, 1H), 7.14 (s, 1H), 7.01-6.99 (m, 2H), 4.57 (s, 2H), 4.33 (q, J=5.20 Hz, 1H), 4.24 (s, 3H), 4.03 (d, J=11.60 Hz, 2H), 3.68 (s, 3H), 3.24 (br, 2H), 3.0-2.80 (m, 2H), 2.61-2.59 (m, 6H), 2.51 (s, 2H), 2.33-2.32 (m, 1H), 2.16-2.14 (m, 2H), 2.10-1.90 (m, 2H), 1.88-1.53 (m, 3H), 1.50-1.22 (m, 2H), 1.01 (d, J=6.00 Hz, 3H).

### Example 69. Synthesis of 2-((6-((5-chloro-2-((3S)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (Compound 308b)

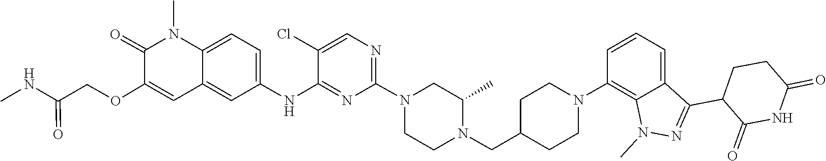 [SMILES: CNC(=O)COc1cc2cc(Nc3nc(N4CCN(CC5CCN(c6cccc7c(C8CCC(=O)NC8=O)nn(C)c67)CC5)[C@@H](C)C4)ncc3Cl)ccc2n(C)c1=O]

### Preparation of tert-butyl(S)-4-((1-((benzyloxy)carbonyl)piperidin-4-yl)methyl)-3-methylpiperazine-1-carboxylate

To a stirred solution of tert-butyl(S)-3-methylpiperazine-1-carboxylate (10 g, 49.93 mmol) in DCM (200 mL), acetic acid (2.99 mL, 49.93 mmol) and benzyl 4-formylpiperidine-1-carboxylate (6.17 g, 24.96 mmol) were added at 0° C. After stirring for 5 hours, sodium triacetoxyborohydride (26.45 g, 124.82 mmol) was added. The reaction was allowed to warm to room temperature over 16 hours. The reaction mixture was quenched with ice-cold saturated ammonium chloride solution (200 mL) and extracted into DCM (400 mL×3). The separated organic layers were washed with brine solution (100 mL), dried over anhydrous Na₂SO₄, filtered, and concentrated under reduced pressure to obtain crude product tert-butyl (S)-4-((1-((benzyloxy)carbonyl)piperidin-4-yl)methyl)-3-methylpiperazine-1-carboxylate (8.0 g) as a light-yellow semi solid.

LC-MS (ESI): m/z=432.54 [M+H]⁺.

### Preparation of tert-butyl(S)-3-methyl-4-(piperidin-4-ylmethyl)piperazine-1-carboxylate

To a stirred solution of tert-butyl(S)-4-((1-((benzyloxy)carbonyl)piperidin-4-yl)methyl)-3-methylpiperazine-1-carboxylate (8.0 g, 18.53 mmol) in THF (120 mL) was added 20% Pd(OH)₂/C (4.0 g, 50% w/w). The reaction was stirred under a hydrogen atmosphere (80 psi) at room temperature for 16 hours. The reaction mixture was diluted with DCM (100 mL) and filtered through a pad of celite. The celite was washed with 30% THF in DCM (300 mL) and the collected filtrate was concentrated under vacuum to obtain crude product. The crude product was washed with n-pentane (30 mL) and dried under vacuum to obtain tert-butyl(S)-3-methyl-4-(piperidin-4-ylmethyl)piperazine-1-carboxylate (6.0 g) as a pale brown solid.

LC-MS (ESI): m/z=297.24 [M+H]⁺.

### Preparation of tert-butyl(S)-4-((1-(3-(2,6-bis(benzyloxy)pyridin-3-yl)-1-methyl-1H-indazol-7-yl)piperidin-4-yl)methyl)-3-methylpiperazine-1-carboxylate

To a stirred solution of 3-(2,6-bis(benzyloxy)pyridin-3-yl)-7-bromo-1-methyl-1H-indazole (1.50 g, 2.99 mmol) in dioxane (7.5 mL) and DMF (1.5 mL) was added tert-butyl(S)-3-methyl-4-(piperidin-4-ylmethyl)piperazine-1-carboxylate (2.67 g, 8.99 mmol) and cesium carbonate (2.93 g, 8.99 mmol). The reaction was degassed with argon for 5 minutes and Pd-PEPPSI-iHeptC1 (0.14 g, 0.15 mmol) was added. The reaction was heated at 100° C. for 16 hours. The reaction mixture was diluted with DCM (50 mL) and filtered through a pad of celite and washed with THF:DCM (30%)(300 mL). The collected filtrate was concentrated under vacuum to obtain crude product. The crude product was purified by flash chromatography (230-400 silica gel; 30-27% EtOAc in petroleum ether) to afford tert-butyl(S)-4-((1-(3-(2,6-bis(benzyloxy)pyridin-3-yl)-1-methyl-1H-indazol-7-yl)piperidin-4-yl)methyl)-3-methylpiperazine-1-carboxylate (0.80 g) as a pale brown semisolid.

LC-MS (ESI): m/z=717.79 [M+H]⁺

### Preparation of tert-butyl (3S)-4-((1-(3-(2,6-dioxopiperidin-3-yl)-1-methyl-1H-indazol-7-yl)piperidin-4-yl)methyl)-3-methylpiperazine-1-carboxylate

To a stirred solution of tert-butyl(S)-4-((1-(3-(2,6-bis(benzyloxy)pyridin-3-yl)-1-methyl-1H-indazol-7-yl)piperidin-4-yl)methyl)-3-methylpiperazine-1-carboxylate (0.71 g, 1.00 mmol) in THF (100 mL) was added 20% Pd(OH)₂/C (0.71 g). The reaction was put under hydrogen atmosphere (80 psi) and stirred at room temperature for 16 hours. The reaction mixture was diluted with DCM (100 mL) and filtered through a celite bed. The celite was washed with 30% THF in DCM (300 mL) and the collected filtrate was concentrated under vacuum to obtain tert-butyl (3S)-4-((1-(3-(2,6-dioxopiperidin-3-yl)-1-methyl-1H-indazol-7-yl)piperidin-4-yl)methyl)-3-methylpiperazine-1-carboxylate (0.65 g) as light-yellow semi-solid.

LC-MS (ESI): m/z=539.41 [M+H]⁺.

### Preparation of 3-(1-methyl-7-(4-(((S)-2-methylpiperazin-1-yl)methyl)piperidin-1-yl)-1H-indazol-3-yl)piperidine-2,6-dione

To a stirred solution of tert-butyl (3S)-4-((1-(3-(2,6-dioxopiperidin-3-yl)-1-methyl-1H-indazol-7-yl)piperidin-4-yl)methyl)-3-methylpiperazine-1-carboxylate (0.65 g, 1.20 mmol) in DCM (10 mL) was added trifluoroacetic acid (5.0 mL). The reaction was stirred at room temperature for 2 hours. The reaction mixture was concentrated under vacuum to get crude 3-(1-methyl-7-(4-(((S)-2-methylpiperazin-1-yl)methyl)piperidin-1-yl)-1H-indazol-3-yl)piperidine-2,6-dione (0.60 g) as a pale brown semisolid.

LC-MS (ESI): m/z=439.57 [M+H]⁺.

### Preparation of 2-((6-((5-chloro-2-((3S)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide

To a stirred solution of 3-(1-methyl-7-(4-(((S)-2-methylpiperazin-1-yl)methyl)piperidin-1-yl)-1H-indazol-3-yl)piperidine-2,6-dione (0.30 g, 0.68 mmol) in DMSO (3.0 mL), DIPEA (3.0 mL) and 2-((6-((2,5-dichloropyrimidin-4-yl)amino)-1-methyl-2-oxo-1,2-dihydroquinolin-3-yl)oxy)-N-methylacetamide (0.14 g, 0.34 mmol) were added. The reaction was heated at 100° C. for 16 hours. The reaction was cooled to room temperature, and the solvent was removed using a centrifugal evaporator to obtain crude product. The crude product was purified by prep HPLC to afford 2-((6-((5-chloro-2-((3S)-4-((1-(3-(2,6-dioxopiperidin-3-yl)-1-methyl-1H-indazol-7-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (0.075 g) as an off-white solid.

LC-MS (ESI): m/z=810.51 [M+H]⁺.

¹H NMR (400 MHZ, DMSO-d₆): δ 10.88 (s, 1H), 8.86 (s, 1H), 8.05 (s, 1H), 7.95 (d, J=2.40 Hz, 2H), 7.74 (dd, J=2.00 Hz, 9.20 Hz, 1H), 7.48 (d, J=8.80 Hz, 1H), 7.37-7.35 (m, 1H), 7.14 (s, 1H), 7.03-7.00 (m, 2H), 4.57 (s, 2H), 4.35-4.31 (m, 1H), 4.24 (s, 3H), 4.03 (d, J=11.60 Hz, 2H), 3.68 (s, 3H), 3.24-3.21 (m, 3H), 2.98-2.90 (m, 1H), 2.87-2.86 (m, 1H), 2.67-2.64 (m, 8H), 2.37-2.31 (m, 2H), 2.19-2.17 (m, 2H), 2.07-2.03 (m, 2H), 1.85-1.65 (m, 2H), 1.41-1.38 (m, 2H), 1.01 (d, J=6.00 Hz, 3H).

### Example 70. Synthesis of 2-((6-((5-chloro-2-((3R)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (Compound 309a)

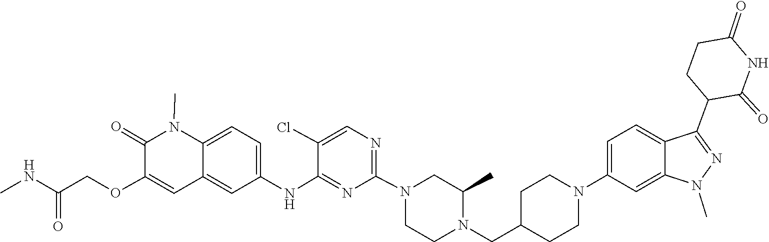 [SMILES: CNC(=O)COc1cc2cc(Nc3nc(N4CCN(CC5CCN(c6ccc7c(C8CCC(=O)NC8=O)nn(C)c7c6)CC5)[C@H](C)C4)ncc3Cl)ccc2n(C)c1=O]

### Preparation of tert-butyl (R)-4-((1-(3-(2,6-bis(benzyloxy)pyridin-3-yl)-1-methyl-1H-indazol-6-yl)piperidin-4-yl)methyl)-3-methylpiperazine-1-carboxylate

To a stirred solution 3-(2,6-bis(benzyloxy)pyridin-3-yl)-6-bromo-1-methyl-1H-indazole (1.5 g, 2.99 mmol) in 1,4-dioxane (30 mL) was added tert-butyl (R)-3-methyl-4-(piperidin-4-ylmethyl)piperazine-1-carboxylate (3.5 g, 12.0 mmol). The reaction was sparged with argon for 15 minutes, then sodium tert-butoxide (0.86 g, 8.97 mmol) and BrettPhos Pd G3 (0.13 g, 0.05 mmol) were added. The reaction was then heated at 80° C. for 16 hours. The reaction mixture was quenched with water and extracted into ethyl acetate. The combined organic layers were washed with brine solution, dried over anhydrous Na₂SO₄, filtered, and concentrated under vacuum to get crude product. The crude product was purified by flash column chromatography (SiO₂, 230-400, 90% ethyl acetate in petroleum ether) to afford tert-butyl (R)-4-((1-(3-(2,6-bis(benzyloxy)pyridin-3-yl)-1-methyl-1H-indazol-6-yl)piperidin-4-yl)methyl)-3-methylpiperazine-1-carboxylate (0.7 g) as a yellow gummy solid.

LC-MS (ESI): m/z=717.79 [M+H]⁺.

### Preparation of tert-butyl (3R)-4-((1-(3-(2,6-dioxopiperidin-3-yl)-1-methyl-1H-indazol-6-yl)piperidin-4-yl)methyl)-3-methylpiperazine-1-carboxylate

To a stirred solution of tert-butyl 4-((1-((benzyloxy)carbonyl)piperidin-4-yl)methyl)piperazine-1-carboxylate (0.70 g, 0.97 mmol) in THF (14 mL) was added 20% Pd(OH)₂ on carbon, moisture 50% wet (0.68 g, 4.88 mmol). The reaction was put under hydrogen atmosphere (60 psi) at room temperature and stirred for 16 hours. The reaction mixture was diluted with DCM and filtered through a celite bed, and the celite was washed with 30% THF in DCM. The collected filtrate was concentrated under vacuum to obtain crude tert-butyl (3R)-4-((1-(3-(2,6-dioxopiperidin-3-yl)-1-methyl-1H-indazol-6-yl)piperidin-4-yl)methyl)-3-methylpiperazine-1-carboxylate (0.4 g) as a light yellow gummy solid.

LC-MS (ESI): m/z=539.59 [M+H]⁺.

### Preparation of 3-(1-methyl-6-(4-(((R)-2-methylpiperazin-1-yl)methyl)piperidin-1-yl)-1H-indazol-3-yl)piperidine-2,6-dione

To a stirred solution of tert-butyl (3R)-4-((1-(3-(2,6-dioxopiperidin-3-yl)-1-methyl-1H-indazol-6-yl)piperidin-4-yl)methyl)-3-methylpiperazine-1-carboxylate (0.40 g, 0.74 mmol) in DCM (4 mL) was added trifluoroacetic acid (4 mL). The reaction was stirred at room temperature for 2 hours. The reaction mixture was concentrated under vacuum to obtain 3-(1-methyl-6-(4-(((R)-2-methylpiperazin-1-yl)methyl)piperidin-1-yl)-1H-indazol-3-yl)piperidine-2,6-dione (0.38 g) as a pale yellow gummy solid.

LC-MS (ESI): m/z=439.53 [M+H]⁺.

### Preparation of 2-((6-((5-chloro-2-((3R)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide

To a stirred solution of 3-(1-methyl-6-(4-(((R)-2-methylpiperazin-1-yl)methyl)piperidin-1-yl)-1H-indazol-3-yl)piperidine-2,6-dione (0.4 g, 0.91 mmol) in DMSO (4.0 mL), 2-((6-((2,5-dichloropyrimidin-4-yl)amino)-1-methyl-2-oxo-1,2-dihydroquinolin-3-yl)oxy)-N-methylacetamide (0.14 g, 0.36 mmol) and DIPEA (1.31 mL, 7.30 mmol) were added. The reaction was heated at 100° C. for 6 hours. The reaction was cooled to room temperature and the solvent was removed using a centrifugal evaporator to obtain crude product. The crude product was purified by prep HPLC to afford 2-((6-((5-chloro-2-((3R)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (0.0305 g) as a light brown solid.

LC-MS (ESI): m/z=810.44 [M+H]⁺.

¹H NMR (400 MHZ, DMSO-d₆): δ 10.84 (s, 1H), 8.86 (s, 1H), 8.05 (s, 1H), 7.94 (s, 2H), 7.74 (d, J=11.2 Hz, 1H), 7.47 (d, J=10.80 Hz, 2H), 7.13 (s, 1H), 6.90 (d, J=9.20 Hz, 1H), 6.82 (s, 1H), 4.57 (s, 2H), 4.26-4.03 (m, 1H), 4.10-4.05 (m, 2H), 4.00 (s, 3H), 3.79 (d, J=12.00 Hz, 2H), 3.68 (s, 3H), 3.32 (s, 1H), 2.98-2.95 (m, 1H), 2.88-2.75 (m, 1H), 2.72-2.61 (m, 8H), 2.36-0.00 (m, 1H), 2.33-2.33 (m, 1H), 2.18-2.01 (m, 1H), 1.90 (s, 1H), 2.01 (s, 1H), 1.75-1.73 (m, 2H), 1.27-1.23 (m, 3H), 0.99-0.97 (m, 3H).

### Example 71. Synthesis of 2-((6-((5-chloro-2-((3S)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (Compound 309b)

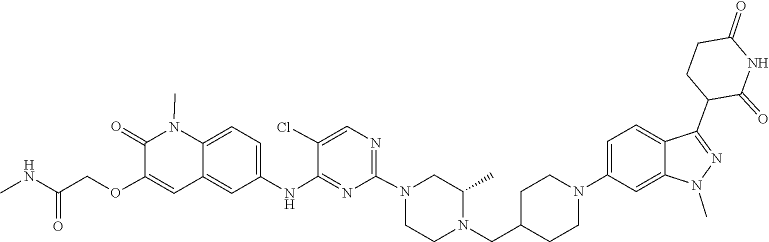 [SMILES: CNC(=O)COc1cc2cc(Nc3nc(N4CCN(CC5CCN(c6ccc7c(C8CCC(=O)NC8=O)nn(C)c7c6)CC5)[C@@H](C)C4)ncc3Cl)ccc2n(C)c1=O]

### Preparation of 2-((6-((5-chloro-2-((3S)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide

2-((6-((5-chloro-2-((3S)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methyl acetamide (0.140 g) was prepared as a pale brown solid in a similar manner as 2-((6-((5-chloro-2-((3R)-4-((1-(3-(2,6-dioxopiperidin-3-yl)-1-methyl-1H-indazol-6-yl)piperidin-4-yl)methyl)-3-methylpiperazin-1-yl)pyrimidin-4-yl)amino)-1-methyl-2-oxo-1,2-dihydroquinolin-3-yl)oxy)-N-methylacetamide (see, e.g., Example 70).

LC-MS (ESI): m/z=810.48 [M+H]⁺.

¹H NMR data (400 MHZ, DMSO-d₆): δ 10.84 (s, 1H), 8.87 (s, 1H), 8.13 (s, 1H), 7.94-7.94 (m, 2H), 7.73 (d, J=8.80 Hz, 1H), 7.48 (d, J=9.20 Hz, 2H), 7.15 (s, 1H), 6.90 (d, J=9.20 Hz, 1H), 6.82 (s, 1H), 4.57 (s, 2H), 4.23-4.24 (m, 1H), 4.01 (br s, 1H), 3.88 (s, 3H), 3.79-3.81 (m, 2H), 3.68 (s, 3H), 3.42-3.47 (m, 1H), 2.99-2.92 (m, 2H), 2.73-2.76 (m, 10H), 2.27-2.28 (m, 2H), 2.18-2.19 (m, 1H), 2.13-2.15 (m, 1H), 1.73-1.75 (m, 3H), 1.22-1.24 (m, 3H), 0.99-0.98 (m, 2H).

### Example 72. Synthesis of 2-((6-((5-chloro-2-(4-(4-((3-(2,6-dioxopiperidin-3-yl)-1-methyl-1H-indazol-6-yl)oxy)butyl)piperidin-1-yl)pyrimidin-4-yl)amino)-1-methyl-2-oxo-1,2-dihydroquinolin-3-yl)oxy)-N-methylacetamide (Compound 319)

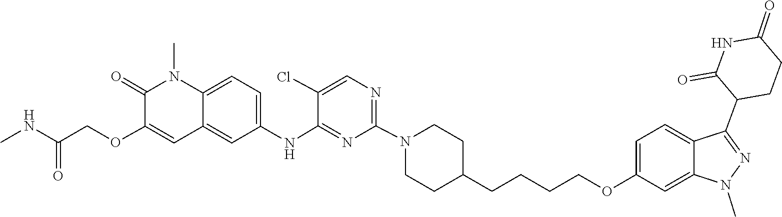 [SMILES: CNC(=O)COc1cc2cc(Nc3nc(N4CCC(CCCCOc5ccc6c(C7CCC(=O)NC7=O)nn(C)c6c5)CC4)ncc3Cl)ccc2n(C)c1=O]

### Preparation of 3-(2,6-bis(benzyloxy)pyridin-3-yl)-1-methyl-1H-indazol-6-ol

To a stirred solution of 3-(2,6-bis(benzyloxy)pyridin-3-yl)-6-bromo-1-methyl-1H-indazole (2.0 g, 4.00 mmol) in 1,4-dioxane (10.0 mL) and water (10.0 mL), potassium hydroxide (0.67 g, 12.0 mmol) was added. The reaction was purged with nitrogen for 15 minutes. Tert-Butyl XPhos (0.19 g, 0.40 mmol) and Pd₂(dba)₃ (0.36 g, 0.40 mmol) were added, and the reaction was stirred at 100° C. 16 hours. The reaction mixture was quenched with water and extracted into ethyl acetate. The combined organic layers were washed with brine solution, dried over anhydrous Na₂SO₄, filtered, and concentrated under vacuum to obtain crude product. The crude product was purified by flash column chromatography (SiO₂, 60-120, 35% ethyl acetate in petroleum ether) to afford 3-(2,6-bis(benzyloxy)pyridin-3-yl)-1-methyl-1H-indazol-6-ol (0.55 g) as a yellow solid.

LC-MS (ESI): m/z=438.37 [M+H]⁺.

### Preparation of tert-butyl 4-(4-((methylsulfonyl)oxy)butyl)piperidine-1-carboxylate

To a stirred solution of tert-butyl 4-(4-hydroxybutyl)piperidine-1-carboxylate (0.5 g, 1.94 mmol) in DCM (20.0 mL), TEA (0.42 mL, 2.91 mmol) was added at 0° C. Methane sulfonyl chloride (0.26 g, 2.33 mmol) was added, and the reaction mixture was allowed to warm to room temperature over 4 hours with stirring. The reaction mixture was quenched with water and extracted into DCM. The combined organic layers were washed with brine solution, dried over anhydrous Na₂SO₄, filtered, and concentrated under vacuum to obtain crude product. The crude product was purified by trituration using diethyl ether to afford tert-butyl 4-(4-((methylsulfonyl)oxy)butyl)piperidine-1-carboxylate (0.70 g) as a yellow semi-solid that was used without further purification in the next reaction.

### Preparation of tert-butyl 4-(4-((3-(2,6-bis(benzyloxy)pyridin-3-yl)-1-methyl-1H-indazol-6-yl)oxy)butyl)piperidine-1-carboxylate

To a stirred solution of 3-(2,6-bis(benzyloxy)pyridin-3-yl)-1-methyl-1H-indazol-6-ol (0.65 g, 1.48 mmol) and tert-butyl 4-(4-((methylsulfonyl)oxy)butyl)piperidine-1-carboxylate (0.59 g, 1.78 mmol) in DMF (13.0 mL), Cs₂CO₃ (1.45 g, 4.46 mmol) was added at 0° C. The reaction was allowed to warm to room temperature over 16 hours with stirring. The reaction mixture was quenched with water and extracted into ethyl acetate. The combined organic layers were washed with brine solution, dried over anhydrous Na₂SO₄, filtered, and concentrated under vacuum to obtain crude product. The crude product was purified by flash column chromatography (SiO₂, 60-120 mesh, 20% ethyl acetate in petroleum ether) to afford tert-butyl 4-(4-((3-(2,6-bis(benzyloxy)pyridin-3-yl)-1-methyl-1H-indazol-6-yl)oxy)butyl)piperidine-1-carboxylate (0.50 g) as a yellow solid.

LC-MS (ESI): m/z=677.72 [M+H]⁺.

### Preparation of tert-butyl 4-(4-((3-(2,6-dioxopiperidin-3-yl)-1-methyl-1H-indazol-6-yl)oxy)butyl)piperidine-1-carboxylate

To a stirred solution of tert-butyl 4-(4-((3-(2,6-bis(benzyloxy)pyridin-3-yl)-1-methyl-1H-indazol-6-yl)oxy)butyl)piperidine-1-carboxylate (0.45 g, 0.66 mmol) in THF (18 mL), 20% Pd(OH)₂ on carbon, moisture 50% wet (0.9 g) was added. The reaction was put under a hydrogen atmosphere (balloon pressure) and stirred at room temperature for 4 hours. The reaction mixture was diluted with EtOAc and filtered through a pad of celite. The celite was washed with 30% THF in EtOAc. The filtrate was concentrated under vacuum to afford tert-butyl 4-(4-((3-(2,6-dioxopiperidin-3-yl)-1-methyl-1H-indazol-6-yl)oxy)butyl)piperidine-1-carboxylate (0.32 g) as an off-white solid.

LC-MS (ESI): m/z=521.55 [M+Na]⁺.

### Preparation of 3-(1-methyl-6-(4-(piperidin-4-yl) butoxy)-1H-indazol-3-yl)piperidine-2,6-dione hydrochloride

To a stirred solution of tert-butyl 4-(4-((3-(2,6-dioxopiperidin-3-yl)-1-methyl-1H-indazol-6-yl)oxy)butyl)piperidine-1-carboxylate (0.32 g, 0.64 mmol) in DCM (3.2 mL), 4 M HCl in dioxane (1.60 mL) was added. The reaction was stirred at room temperature for 3 hours. The reaction mixture was concentrated under vacuum to obtain crude product. The crude product was triturated using diethyl ether to afford 3-(1-methyl-6-(4-(piperidin-4-yl) butoxy)-1H-indazol-3-yl)piperidine-2,6-dione hydrochloride (0.27 g).

LC-MS (ESI): m/z=399.48 [M+H]⁺.

### Preparation of 2-((6-((5-chloro-2-(4-(4-((3-(2,6-dioxopiperidin-3-yl)-1-methyl-1H-indazol-6-yl)oxy)butyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(4-(piperidin-4-yl) butoxy)-1H-indazol-3-yl)piperidine-2,6-dione hydrochloride (0.27 g, 0.62 mmol) in DMSO (10.8 mL), 2-((6-((2,5-dichloropyrimidin-4-yl)amino)-1-methyl-2-oxo-1,2-dihydroquinolin-3-yl)oxy)-N-methylacetamide (0.15 g, 0.37 mmol) and DIPEA (0.45 mL, 3.1 mmol) were added. The reaction was heated to 100° C. for 5 hours. The reaction mixture was poured into ice-cold water, and the resulting precipitate was filtered to obtain crude product. The crude product was purified by preparative HPLC to afford 2-((6-((5-chloro-2-(4-(4-((3-(2,6-dioxopiperidin-3-yl)-1-methyl-1H-indazol-6-yl)oxy)butyl)piperidin-1-yl)pyrimidin-4-yl)amino)-1-methyl-2-oxo-1,2-dihydroquinolin-3-yl)oxy)-N-methylacetamide (0.052 g) as an off-white solid.

LC-MS (ESI): m/z=768.43 [M−H]⁻.

¹H NMR (400 MHZ, DMSO-d₆): δ 10.85 (s, 1H), 8.82 (s, 1H), 8.03 (s, 1H), 7.96-7.92 (m, 2H), 7.76-7.73 (m, 1H), 7.55-7.53 (d, J=8.8 Hz, 1H), 7.48-7.46 (d, J-9.2 Hz, 1H), 7.09 (s, 1H), 7.038-7.034 (d, J=1.6 Hz, 1H), 6.73-6.70 (m, 1H), 4.51-4.47 (m, 4H), 4.30-4.327 (m, 1H), 4.06-4.03 (t, J=6.4 Hz, 2H), 3.91 (s, 3H), 3.32 (s, 3H), 2.84-2.78 (m, 2H), 2.67-2.55 (m, 5H), 2.33-2.29 (m, 1H), 2.18-2.14 (m, 1H), 1.77-1.73 (m, 4H), 1.52-1.50 (m, 3H), 1.32-1.30 (m, 2H), 1.05-1.03 (m, 2H).

### Example 73. Synthesis of 2-((6-((5-chloro-2-(4-((1-(3-(2,6-dioxopiperidin-3-yl)-1-methyl-1H-indazol-7-yl)pyrrolidin-3-yl)methyl)piperidin-1-yl)pyrimidin-4-yl)amino)-1-methyl-2-oxo-1,2-dihydroquinolin-3-yl)oxy)-N-methylacetamide (Compound 320)

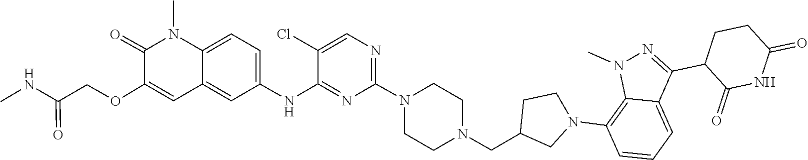 [SMILES: CNC(=O)COc1cc2cc(Nc3nc(N4CCN(CC5CCN(c6cccc7c(C8CCC(=O)NC8=O)nn(C)c67)C5)CC4)ncc3Cl)ccc2n(C)c1=O]

### Preparation of tert-butyl 3-(pyridin-4-ylmethyl)pyrrolidine-1-carboxylate

A stirred solution of tert-butyl 3-methylenepyrrolidine-1-carboxylate (5.0 g, 27.3 mmol) in THF (130 mL) was degassed with nitrogen for 5 minutes. 9-BBN 0.5 M in THF (50.0 mL, 27.3 mmol) was added, and the resulting mixture was heated at 65° C. for 1 hour. After cooling to room temperature, [1,1′-Bis(diphenylphosphino) ferrocene]palladium (II) dichloride (0.99 g, 1.36 mmol) and potassium phosphate (17.0 g, 81.9 mmol) were added a solution in THF (10.0 mL). The reaction was heated 60° C. for 3 hours. The reaction mixture was quenched with water (40 mL) and extracted into EtOAc (2×130 mL). The combined organic layers were dried over anhydrous sodium sulfate, filtered, and concentrated to obtain crude product. The crude product was purified by flash column chromatography (SiO₂, 100-200, 80-90% ethyl acetate in petroleum ether) to afford tert-butyl 3-(pyridin-4-ylmethyl)pyrrolidine-1-carboxylate (4.0 g) as a brown sticky solid.

LC-MS (ESI): m/z=263.37 [M+H]⁺.

### Preparation of tert-butyl 3-(piperidin-4-ylmethyl)pyrrolidine-1-carboxylate

To a stirred solution of tert-butyl 3-(pyridin-4-ylmethyl)pyrrolidine-1-carboxylate (4.0 g, 15.0 mmol) in acetic acid (80.0 mL), platinum dioxide (0.38 g, 1.67 mmol) was added under an inert atmosphere. The reaction was put under a hydrogen atmosphere (30 psi) and stirred for 16 hours at room temperature. The reaction mixture was filtered through a pad of celite and washed with acetic acid (10 mL). The filtrate was concentrated to afford tert-butyl 3-(piperidin-4-ylmethyl)pyrrolidine-1-carboxylate (4.0 g) as a yellow, gummy solid.

LC-MS (ESI): m/z=269.39 [M+H]⁺.

### Preparation of benzyl 4-((1-(tert-butoxycarbonyl)pyrrolidin-3-yl)methyl)piperidine-1-carboxylate

To a stirred solution of tert-butyl 3-(piperidin-4-ylmethyl)pyrrolidine-1-carboxylate (4.0 g, 15.0 mmol) in DCM (120 mL), triethylamine (6.2 mL, 44.7 mmol) and benzyl chloroformate (3.2 mL, 22.4 mmol) were added at 0° C. under a nitrogen atmosphere. The reaction was allowed to warm to room temperature over 4 hours with stirring. The reaction mixture was concentrated under reduced pressure and co-distilled with DCM (10 mL) to obtain crude product. The crude was purified by triturating with diethyl ether (10 mL) and n-pentane (10 mL) to afford benzyl 4-((1-(tert-butoxycarbonyl)pyrrolidin-3-yl)methyl)piperidine-1-carboxylate (4.5 g) as an off-white solid.

LC-MS (ESI): m/z=425.7 [M+Na]⁺.

### Preparation of benzyl 4-(pyrrolidin-3-ylmethyl)piperidine-1-carboxylate

To a stirred solution of benzyl 4-((1-(tert-butoxycarbonyl)pyrrolidin-3-yl)methyl)piperidine-1-carboxylate (4.0 g, 0.955 mmol) in DCM (4.0 mL), 4 M HCl in dioxane (2.0 mL) was added at 0° C. under a nitrogen atmosphere. The reaction was allowed to warm to room temperature over 3 hours with stirring. The reaction mixture was concentrated under reduced pressure and co-distilled with DCM (10 mL) to obtain crude product. The crude product was purified by triturating with diethyl ether (10 mL) and n-pentane (10 mL) to afford benzyl 4-(pyrrolidin-3-ylmethyl)piperidine-1-carboxylate (3.0 g) as a brown solid.

LC-MS (ESI): m/z=303.38 [M+H]⁺.

### Preparation of benzyl 4-((1-(3-(2,6-bis(benzyloxy)pyridin-3-yl)-1-methyl-1H-indazol-7-yl)pyrrolidin-3-yl)methyl)piperidine-1-carboxylate

A solution of benzyl 4-(pyrrolidin-3-ylmethyl)piperidine-1-carboxylate (0.6 g, 1.98 mmol), 3-(2,6-bis(benzyloxy)pyridin-3-yl)-7-bromo-1-methyl-1H-indazole (0.79 g, 1.58 mmol), and sodium tert-butoxide (0.38 g, 3.96 mmol) in 1,4-dioxane (18 mL) was degassed with nitrogen for 15 minutes. Pd-PEPPSI-IHeptCl (0.096 g, 0.09 mmol) was added, and the reaction was heated at 70° C. for 3 hours. The reaction was quenched with water (50 mL) and extracted with ethyl acetate (10 mL×2). The combined organic layers were washed with brine (10 mL), dried over sodium sulfate, filtered, and concentrated under reduced pressure to obtain crude product. The crude product was purified by flash column chromatography (SiO₂, 100-200, 30-40% ethyl acetate in petroleum ether) to afford benzyl 4-((1-(3-(2,6-bis(benzyloxy)pyridin-3-yl)-1-methyl-1H-indazol-7-yl)pyrrolidin-3-yl)methyl)piperidine-1-carboxylate (0.31 g) as a grey solid.

LC-MS (ESI): m/z=722.73 [M+H]⁺.

### Preparation of 3-(1-methyl-7-(3-(piperidin-4-ylmethyl)pyrrolidin-1-yl)-1H-indazol-3-yl)piperidine-2,6-dione hydrochloride

A stirred solution of benzyl 4-((1-(3-(2,6-bis(benzyloxy)pyridin-3-yl)-1-methyl-1H-indazol-7-yl)pyrrolidin-3-yl)methyl)piperidine-1-carboxylate (0.30 g, 0.41 mmol) in tetrahydrofuran (3 mL), ethyl acetate (3 mL), and acetic acid (0.6 mL) was put under a nitrogen atmosphere. 20% Palladium hydroxide on carbon (0.15 g, 50% w/w) was added. The reaction was put under a hydrogen atmosphere (30 psi) and stirred at room temperature for 24 hours. The reaction mixture was filtered through a pad of celite, and the celite was washed with THF (10 mL). The filtrate was concentrated to afford 3-(1-methyl-7-(3-(piperidin-4-ylmethyl)pyrrolidin-1-yl)-1H-indazol-3-yl)piperidine-2,6-dione hydrochloride (0.12 g) as a brown solid.

LC-MS (ESI): m/z=410.49 [M+H]⁺.

### Preparation of 2-((6-((5-chloro-2-(4-((1-(3-(2,6-dioxopiperidin-3-yl)-1-methyl-1H-indazol-7-yl)pyrrolidin-3-yl)methyl)piperidin-1-yl)pyrimidin-4-yl)amino)-1-methyl-2-oxo-1,2-dihydroquinolin-3-yl)oxy)-N-methylacetamide

To a stirred solution of 2-((6-((2,5-dichloropyrimidin-4-yl)amino)-1-methyl-2-oxo-1,2-dihydroquinolin-3-yl)oxy)-N-methylacetamide (0.084 g, 0.20 mmol) in DMSO (2.5 mL), DIPEA (0.15 mL, 0.87 mmol) was added. After stirring for 10 minutes, 3-(1-methyl-7-(3-(piperidin-4-ylmethyl)pyrrolidin-1-yl)-1H-indazol-3-yl)piperidine-2,6-dione hydrochloride (0.12 g, 0.29 mmol) was added. The reaction was heated to 120° C. for 3 hours. The reaction mixture was quenched with cold water (5 mL). The resulting precipitate was filtered and washed with pentane (10 mL) to obtain crude product. The crude product was purified by prep-HPLC to afford 2-((6-((5-chloro-2-(4-((1-(3-(2,6-dioxopiperidin-3-yl)-1-methyl-1H-indazol-7-yl)pyrrolidin-3-yl)methyl)piperidin-1-yl)pyrimidin-4-yl)amino)-1-methyl-2-oxo-1,2-dihydroquinolin-3-yl)oxy)-N-methylacetamide (0.024 g) as a grey solid.

LC-MS (ESI): m/z=781.51 [M+H]⁺.

¹H NMR (400 MHZ, DMSO-d₆): δ 10.9 (s, 1H), 8.81 (s, 1H), 8.03 (s, 1H), 7.95-7.93 (m, 2H), 7.75 (dd, J=2.00, 9.20 Hz, 1H), 7.47 (d, J=9.20 Hz, 1H), 7.33 (d, J=7.60 Hz, 1H), 7.09 (s, 1H), 7.03-6.97 (m, 2H), 6.71 (s, 1H), 4.51-4.49 (m, 4H), 4.48-4.34 (d, 1H), 4.33-4.30 (m, 1H), 4.18 (s, 3H), 3.67 (s, 3H), 3.24-3.07 (m, 3H), 2.85-2.61 (m, 9H), 2.32 (br s, 1H), 2.18-2.15 (m, 2H), 1.75-1.73 (m, 2H), 1.59-1.54 (m, 2H), 1.42-1.40 (m, 2H), 1.05-1.01 (m, 2H).

### Example 74. Synthesis of 2-((6-((5-chloro-2-(3-((1-(3-(2,6-dioxopiperidin-3-yl)-1-methyl-1H-indazol-7-yl)piperidin-4-yl)methyl)-3,8-diazabicyclo[3.2.1]octan-8-yl)pyrimidin-4-yl)amino)-1-methyl-2-oxo-1,2-dihydroquinolin-3-yl)oxy)-N-methylacetamide (Compound 325)

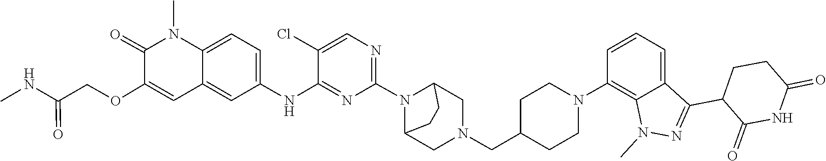 [SMILES: CNC(=O)COc1cc2cc(Nc3nc(N4C5CCC4CN(CC4CCN(c6cccc7c(C8CCC(=O)NC8=O)nn(C)c67)CC4)C5)ncc3Cl)ccc2n(C)c1=O]

### Preparation of tert-butyl 3-((1-((benzyloxy)carbonyl)piperidin-4-yl)methyl)-3,8-diazabicyclo[3.2.1]octane-8-carboxylate

To a stirred solution of tert-butyl 3,8-diazabicyclo[3.2.1]octane-8-carboxylate (3.00 g, 14.1 mmol) and benzyl 4-formylpiperidine-1-carboxylate (4.20 g, 17.0 mmol) in DCM (30 mL), acetic acid (0.82 mL, 14.1 mmol) was added. After stirring for 1 hour, sodium triacetoxyborohydride (6.00 g, 28.3 mmol) was added. The reaction was stirred at room temperature for 16 hours. The reaction mixture was quenched with ammonium chloride solution (70 mL) and extracted with DCM (2×80 mL). The combined organic layers were washed with brine solution (50 mL), dried over anhydrous Na₂SO₄, filtered, and concentrated under reduced pressure to obtain tert-butyl 3-((1-((benzyloxy)carbonyl)piperidin-4-yl)methyl)-3,8-diazabicyclo[3.2.1]octane-8-carboxylate (5.81 g) as a pale yellow liquid.

LC-MS (ESI): m/z=444.55 [M+H]⁺.

### Preparation of tert-butyl 3-(piperidin-4-ylmethyl)-3,8-diazabicyclo[3.2.1]octane-8-carboxylate

To a solution of tert-butyl 3-((1-((benzyloxy)carbonyl)piperidin-4-yl)methyl)-3,8-diazabicyclo[3.2.1]octane-8-carboxylate (3.00 g, 6.76 mmol) in THF (120 mL), 20% Pd(OH)₂/C (1.80 g, 60% w/w) was added. The reaction was put under hydrogen atmosphere (80 psi) and stirred at room temperature for 16 hours. The reaction mixture was diluted with THF (50 mL) and filtered through a celite bed. The celite was washed with 200 mL of THF:DCM (1:1). The filtrate was concentrated and dried to afford tert-butyl 3-(piperidin-4-ylmethyl)-3,8-diazabicyclo[3.2.1]octane-8-carboxylate (2.01 g) as a pale yellow gummy compound.

LC-MS (ESI): m/z=310.21 [M+H]⁺.

### Preparation of tert-butyl 3-((1-(3-(2,6-bis(benzyloxy)pyridin-3-yl)-1-methyl-1H-indazol-7-yl)piperidin-4-yl)methyl)-3,8-diazabicyclo[3.2.1]octane-8-carboxylate

To a stirred solution of 3-(2,6-bis(benzyloxy)pyridin-3-yl)-7-bromo-1-methyl-1H-indazole (1.00 g, 1.99 mmol) and tert-butyl 3-(piperidin-4-ylmethyl)-3,8-diazabicyclo[3.2.1]octane-8-carboxylate (1.55 g, 4.99 mmol) in 1,4-dioxane (14 mL), cesium carbonate (1.95 g, 5.99 mmol) was added. The reaction was degassed with argon for 15 minutes, then Pd-PEPPSI-IHeptCl (0.10 g, 0.10 mmol) was added, and the reaction was stirred at 100° C. for 16 hours. The reaction mixture was diluted with ethyl acetate (50 mL) and filtered through a celite bed. The celite was washed with 10% MeOH in DCM (150 mL). The filtrate was concentrated under vacuum to obtain crude product. The crude was purified by flash chromatography (230-400 silica gel; 10-15% EtOAc in petroleum ether) to afford tert-butyl 3-((1-(3-(2,6-bis(benzyloxy)pyridin-3-yl)-1-methyl-1H-indazol-7-yl)piperidin-4-yl)methyl)-3,8-diazabicyclo[3.2.1]octane-8-carboxylate (0.80 g) as a pale yellow solid.

LC-MS (ESI): m/z=729.54 [M+H]⁺.

### Preparation of tert-butyl 3-((1-(3-(2,6-dioxopiperidin-3-yl)-1-methyl-1H-indazol-7-yl)piperidin-4-yl)methyl)-3,8-diazabicyclo[3.2.1]octane-8-carboxylate

To a solution of tert-butyl 3-((1-(3-(2,6-bis(benzyloxy)pyridin-3-yl)-1-methyl-1H-indazol-7-yl)piperidin-4-yl)methyl)-3,8-diazabicyclo[3.2.1]octane-8-carboxylate (0.78 g, 1.07 mmol) in THF (32 mL), 20% Pd(OH)₂/C (0.78 g, 100% w/w) was added. The reaction was put under hydrogen atmosphere (80 psi) and stirred at room temperature for 5 hours. The reaction mixture was diluted with THF (50 mL) and filtered through a celite bed. The celite was washed with THF:DCM (1:1, 150 mL), and the filtrate was concentrated to afford tert-butyl 3-((1-(3-(2,6-dioxopiperidin-3-yl)-1-methyl-1H-indazol-7-yl)piperidin-4-yl)methyl)-3,8-diazabicyclo[3.2.1]octane-8-carboxylate (0.59 g) as a pale brown solid.

LC-MS (ESI): m/z=551.68 [M+H]⁺.

### Preparation of 3-(7-(4-((3,8-diazabicyclo[3.2.1]octan-3-yl)methyl)piperidin-1-yl)-1-methyl-1H-indazol-3-yl)piperidine-2,6-dione

A stirred solution of tert-butyl 3-((1-(3-(2,6-dioxopiperidin-3-yl)-1-methyl-1H-indazol-7-yl)piperidin-4-yl)methyl)-3,8-diazabicyclo[3.2.1]octane-8-carboxylate (0.59 g, 1.07 mmol) in DCM (6 mL) was cooled to 0° C. TFA (3.0 mL) was added, and the reaction was allowed to warm to room temperature over 3 hours with stirring. The reaction mixture was concentrated under reduced pressure and residual solvent was co-distilled with DCM (3×10 mL) to afford 3-(7-(4-((3,8-diazabicyclo[3.2.1]octan-3-yl)methyl)piperidin-1-yl)-1-methyl-1H-indazol-3-yl)piperidine-2,6-dione (0.51 g) as an off-white solid.

LC-MS (ESI): m/z=451.29 [M+H]⁺.

### Preparation of 2-((6-((5-chloro-2-(3-((1-(3-(2,6-dioxopiperidin-3-yl)-1-methyl-1H-indazol-7-yl)piperidin-4-yl)methyl)-3,8-diazabicyclo[3.2.1]octan-8-yl)pyrimidin-4-yl)amino)-1-methyl-2-oxo-1,2-dihydroquinolin-3-yl)oxy)-N-methylacetamide

To a stirred solution of 3-(7-(4-((3,8-diazabicyclo[3.2.1]octan-3-yl)methyl)piperidin-1-yl)-1-methyl-1H-indazol-3-yl)piperidine-2,6-dione (0.25 g, 0.55 mmol) and DIPEA (2.0 mL) in DMSO (2.0 mL), 2-((6-((2,5-dichloropyrimidin-4-yl)amino)-1-methyl-2-oxo-1,2-dihydroquinolin-3-yl)oxy)-N-methylacetamide (0.09 g, 0.22 mmol) was added. The reaction was stirred at 100° C. for 16 hours. The reaction mixture was concentrated using a centrifugal evaporator to obtain crude product. The crude product was purified by prep-HPLC to afford 2-((6-((5-chloro-2-(3-((1-(3-(2,6-dioxopiperidin-3-yl)-1-methyl-1H-indazol-7-yl)piperidin-4-yl)methyl)-3,8-diazabicyclo[3.2.1]octan-8-yl)pyrimidin-4-yl)amino)-1-methyl-2-oxo-1,2-dihydroquinolin-3-yl)oxy)-N-methylacetamide (0.07 g) as an off-white solid.

LC-MS (ESI): m/z=820.47 [M−H]⁻.

¹H NMR (400 MHZ, DMSO-d₆): δ 10.89 (s, 1H), 8.86 (s, 1H), 8.06 (s, 1H), 7.98-7.95 (m, 2H), 7.78 (dd, J=2.40, 9.20 Hz, 1H), 7.49 (d, J=9.20 Hz, 1H), 7.37-7.35 (m, 1H), 7.05-7.00 (m, 3H), 4.63-4.47 (m, 4H), 4.33 (q, J=5.20 Hz, 1H), 4.24 (s, 3H), 3.68 (s, 3H), 3.27-3.19 (m, 2H), 2.71-2.62 (m, 9H), 2.34-2.13 (m, 6H), 1.92-1.71 (m, 7H), 1.38-1.35 (m, 2H).

### Example 75. Synthesis of 2-((6-((5-chloro-2-(2-(3-(2,6-dioxopiperidin-3-yl)-1-methyl-1H-indazol-7-yl) octahydro-5H-pyrrolo[3,4-c]pyridin-5-yl)pyrimidin-4-yl)amino)-1-methyl-2-oxo-1,2-dihydroquinolin-3-yl)oxy)-N-methylacetamide (Compound 326)

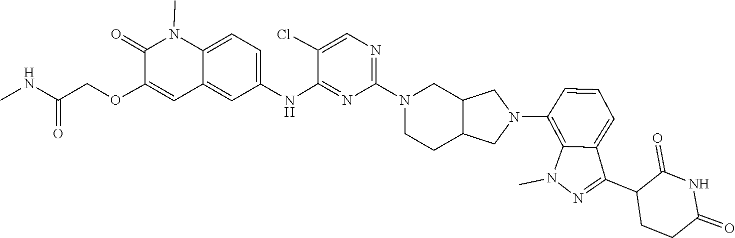 [SMILES: CNC(=O)COc1cc2cc(Nc3nc(N4CCC5CN(c6cccc7c(C8CCC(=O)NC8=O)nn(C)c67)CC5C4)ncc3Cl)ccc2n(C)c1=O]

### Preparation of 2-((6-((5-chloro-2-(2-(3-(2,6-dioxopiperidin-3-yl)-1-methyl-1H-indazol-7-yl) octahydro-5H-pyrrolo[3,4-c]pyridin-5-yl)pyrimidin-4-yl)amino)-1-methyl-2-oxo-1,2-dihydroquinolin-3-yl)oxy)-N-methylacetamide

2-((6-((5-chloro-2-(2-(3-(2,6-dioxopiperidin-3-yl)-1-methyl-1H-indazol-7-yl) octahydro-5H-pyrrolo[3,4-c]pyridin-5-yl)pyrimidin-4-yl)amino)-1-methyl-2-oxo-1,2-dihydroquinolin-3-yl)oxy)-N-methylacetamide (0.165 g) was prepared as an off-white solid in a similar manner as 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (see, e.g., Example 44).

LC-MS (ESI): m/z=739.47 [M+H]⁺.

¹H NMR (400 MHZ, DMSO-d₆): δ 10.90 (s, 1H), 8.82 (s, 1H), 8.06 (s, 1H), 7.99 (br s, 1H), 7.93 (d, J=5.20 Hz, 1H), 7.81 (d, J=9.20 Hz, 1H), 7.46 (d, J=9.20 Hz, 1H), 7.33 (d, J=7.60 Hz, 1H), 7.10-7.05 (m, 2H), 7.00-6.96 (m, 1H), 6.99-6.97 (m, 1H), 4.55 (s, 2H), 4.33-4.30 (m, 1H), 4.09 (d, J=6.00 Hz, 3H), 3.75-3.65 (m, 3H), 3.66 (s, 3H), 3.32-3.20 (m, 3H), 2.99-2.93 (m, 2H), 2.67-2.63 (m, 6H), 2.45-2.35 (m, 1H), 2.20-2.12 (m, 1H), 1.95-1.85 (m, 1H), 1.82-1.75 (m, 1H).

### Example 76. Synthesis of 2-((6-((5-chloro-2-(2-(3-(2,6-dioxopiperidin-3-yl)-1-methyl-1H-indazol-6-yl)-2,6-diazaspiro[3.5]nonan-6-yl)pyrimidin-4-yl)amino)-1-methyl-2-oxo-1,2-dihydroquinolin-3-yl)oxy)-N-methylacetamide (Compound 329)

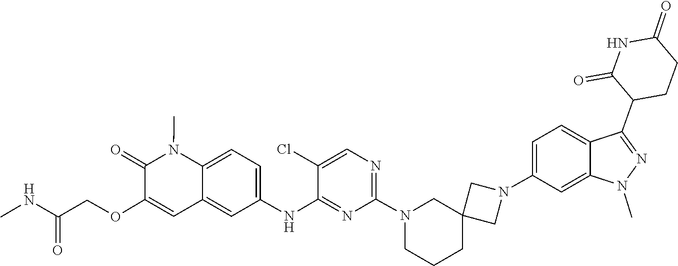 [SMILES: CNC(=O)COc1cc2cc(Nc3nc(N4CCCC5(CN(c6ccc7c(C8CCC(=O)NC8=O)nn(C)c7c6)C5)C4)ncc3Cl)ccc2n(C)c1=O]

### Preparation of tert-butyl 2-(3-(2,6-bis(benzyloxy)pyridin-3-yl)-1-methyl-1H-indazol-6-yl)-2,6-diazaspiro[3.5]nonane-6-carboxylate

To a stirred solution of 3-(2,6-bis(benzyloxy)pyridin-3-yl)-6-bromo-1-methyl-1H-indazole (1.0 g, 1.99 mmol) and tert-butyl 2,6-diazaspiro[3.5]nonane-6-carboxylate (0.68 g, 0.99 mmol) in 1,4-dioxane (20.0 mL) was added cesium carbonate (1.95 g, 5.99 mmol). The reaction was sparged with argon for 10 minutes, then RuPhos (0.09 g, 0.20 mmol) and RuPhos Pd G3 (0.08 g, 0.10 mmol) were added, and the reaction was sparged for an additional 5 minutes. The reaction mixture was stirred at 80° C. for 3 hours. The reaction mixture was cooled to room temperature and filtered through a celite bed. The filtrate was concentrated to get the crude product. The crude product was purified by flash column chromatography (SiO₂, 230-400, 35% ethyl acetate in petroleum ether) to afford tert-butyl 2-(3-(2,6-bis(benzyloxy)pyridin-3-yl)-1-methyl-1H-indazol-6-yl)-2,6-diazaspiro[3.5]nonane-6-carboxylate (1.20 g) as an off-white solid.

LC-MS (ESI): m/z=646.68 [M+H]⁺.

### Preparation of tert-butyl 2-(3-(2,6-dioxopiperidin-3-yl)-1-methyl-1H-indazol-6-yl)-2,6-diazaspiro[3.5]nonane-6-carboxylate

To a stirred solution of tert-butyl 2-(3-(2,6-bis(benzyloxy)pyridin-3-yl)-1-methyl-1H-indazol-6-yl)-2,6-diazaspiro[3.5]nonane-6-carboxylate (1.0 g, 1.55 mmol) in THF (40 mL) was added 20% Pd(OH)₂ (1.0 g) at room temperature. The reaction was stirred under hydrogen (60 psi) atmosphere for 8 hours. The reaction mixture was filtered through a celite bed, then the celite was washed with ethyl acetate, and the filtrate was concentrated to afford crude product. The crude product was washed with n-pentane and dried to afford tert-butyl 2-(3-(2,6-dioxopiperidin-3-yl)-1-methyl-1H-indazol-6-yl)-2,6-diazaspiro[3.5]nonane-6-carboxylate (0.72 g) as a brown solid.

LC-MS (ESI): m/z=468.58 [M+H]⁺.

### Preparation of 3-(1-methyl-6-(2,6-diazaspiro[3.5]nonan-2-yl)-1H-indazol-3-yl)piperidine-2,6-dione

To a stirred solution of tert-butyl 2-(3-(2,6-dioxopiperidin-3-yl)-1-methyl-1H-indazol-6-yl)-2,6-diazaspiro[3.5]nonane-6-carboxylate (0.80 g, 1.71 mmol) in DCM (8 mL) at 0° C. was added TFA (8 mL). The reaction mixture was allowed to warm to room temperature over 3 hours. The reaction mixture was concentrated to get the crude product. The crude product was triturated with diethyl ether (3 times), and the solvent was removed to afford 3-(1-methyl-6-(2,6-diazaspiro[3.5]nonan-2-yl)-1H-indazol-3-yl)piperidine-2,6-dione (0.70 g) as a brown solid.

LC-MS (ESI): m/z=368.18 [M+H]⁺.

### Preparation of 2-((6-((5-chloro-2-(2-(3-(2,6-dioxopiperidin-3-yl)-1-methyl-1H-indazol-6-yl)-2,6-diazaspiro[3.5]nonan-6-yl)pyrimidin-4-yl)amino)-1-methyl-2-oxo-1,2-dihydroquinolin-3-yl)oxy)-N-methylacetamide

To a stirred solution of 3-(1-methyl-6-(2,6-diazaspiro[3.5]nonan-2-yl)-1H-indazol-3-yl)piperidine-2,6-dione (0.40 g, 1.08 mmol) and 2-((6-((2,5-dichloropyrimidin-4-yl)amino)-1-methyl-2-oxo-1,2-dihydroquinolin-3-yl)oxy)-N-methylacetamide (0.18 g, 0.43 mmol) in DMSO (8 mL), N,N-diisopropylethylamine (1.5 mL, 8.71 mmol) was added. The reaction mixture was stirred at 90° C. for 16 hours. The reaction was cooled to room temperature, and the solvent was evaporated using a centrifugal evaporator to obtain crude product. The crude product was purified by prep-HPLC to afford 2-((6-((5-chloro-2-(2-(3-(2,6-dioxopiperidin-3-yl)-1-methyl-1H-indazol-6-yl)-2,6-diazaspiro[3.5]nonan-6-yl)pyrimidin-4-yl)amino)-1-methyl-2-oxo-1,2-dihydroquinolin-3-yl)oxy)-N-methylacetamide (118 mg) as a gray solid.

LC-MS (ESI): m/z=739.39 [M+H]⁺.

¹H NMR (400 MHZ, DMSO-d₆): δ 10.82 (s, 1H), 8.86 (s, 1H), 8.09 (s, 1H), 7.91-7.45 (m, 3H), 7.46 (d, J=8.80 Hz, 1H), 7.08 (s, 1H), 6.71-5.91 (m, 3H), 4.50 (s, 2H), 4.23-4.22 (m, 1H), 3.89-3.49 (m, 12H), 2.92 (br, 2H), 2.65-2.54 (m, 5H), 2.32-2.11 (m, 2H), 1.92-1.81 (br s, 2H), 1.52-1.49 (m, 2H).

### Example 77. Synthesis of 2-((6-((5-chloro-2-(8-(3-(2,6-dioxopiperidin-3-yl)-1-methyl-1H-indazol-7-yl)-2,8-diazaspiro[5.5]undecan-2-yl)pyrimidin-4-yl)amino)-1-methyl-2-oxo-1,2-dihydroquinolin-3-yl)oxy)-N-methylacetamide (Compound 330)

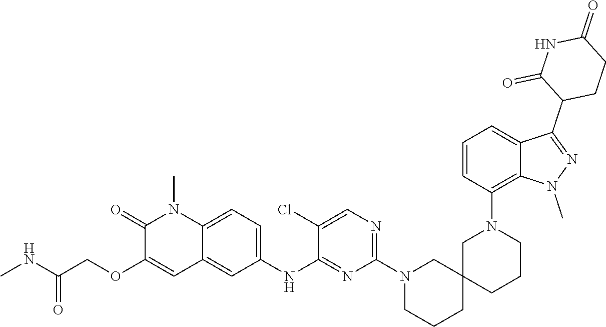 [SMILES: CNC(=O)COc1cc2cc(Nc3nc(N4CCCC5(CCCN(c6cccc7c(C8CCC(=O)NC8=O)nn(C)c67)C5)C4)ncc3Cl)ccc2n(C)c1=O]

### Preparation of 2-((6-((5-chloro-2-(8-(3-(2,6-dioxopiperidin-3-yl)-1-methyl-1H-indazol-7-yl)-2,8-diazaspiro[5.5]undecan-2-yl)pyrimidin-4-yl)amino)-1-methyl-2-oxo-1,2-dihydroquinolin-3-yl)oxy)-N-methylacetamide

2-((6-((5-chloro-2-(8-(3-(2,6-dioxopiperidin-3-yl)-1-methyl-1H-indazol-7-yl)-2,8-diazaspiro[5.5]undecan-2-yl)pyrimidin-4-yl)amino)-1-methyl-2-oxo-1,2-dihydroquinolin-3-yl)oxy)-N-methylacetamide (0.071 g) was prepared as an off-white solid in a similar manner as 2-((6-((5-chloro-2-((((1s,4s)-4-(((3-(2,6-dioxopiperidin-3-yl)-1-methyl-1H-indazol-7-yl)amino)methyl)cyclohexyl)methyl)amino)pyrimidin-4-yl)amino)-1-methyl-2-oxo-1,2-dihydroquinolin-3-yl)oxy)-N-methylacetamide (see, e.g., Example 44).

LC-MS (ESI): m/z=767.52 [M+H]⁺.

¹H NMR (400 MHZ, DMSO-d₆): δ 10.87 (s, 1H), 8.80 (d, J=15.60 Hz, 1H), 8.02-7.61 (m, 4H), 7.72 (s, 1H), 7.47-7.35 (m, 2H), 7.12-6.78 (m, 3H), 4.53 (s, 3H), 4.32-4.23 (m, 3H), 3.90-3.47 (m, 7H), 2.95 (s, 1H), 2.66-2.54 (m, 6H), 2.39-2.07 (m, 3H), 1.99-1.89 (m, 1H), 1.75-1.46 (m, 7H).

### Example 78. Synthesis of 2-((6-((5-chloro-2-(2-(3-(2,6-dioxopiperidin-3-yl)-1-methyl-1H-indazol-6-yl)-2,7-diazaspiro[4.5]decan-7-yl)pyrimidin-4-yl)amino)-1-methyl-2-oxo-1,2-dihydroquinolin-3-yl)oxy)-N-methylacetamide (Compound 331)

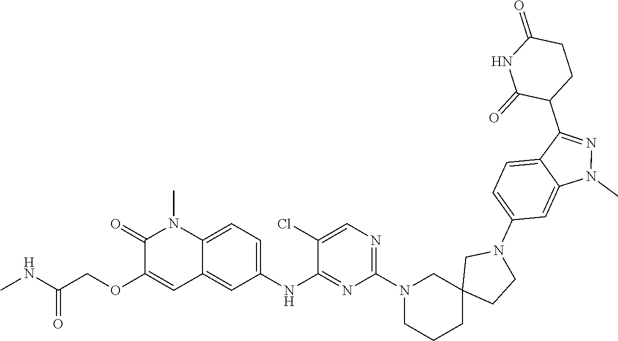

### Preparation of 2-((6-((5-chloro-2-(2-(3-(2,6-dioxopiperidin-3-yl)-1-methyl-1H-indazol-6-yl)-2,7-diazaspiro[4.5]decan-7-yl)pyrimidin-4-yl)amino)-1-methyl-2-oxo-1,2-dihydroquinolin-3-yl)oxy)-N-methylacetamide

2-((6-((5-chloro-2-(2-(3-(2,6-dioxopiperidin-3-yl)-1-methyl-1H-indazol-6-yl)-2,7-diazaspiro[4.5]decan-7-yl)pyrimidin-4-yl)amino)-1-methyl-2-oxo-1,2-dihydroquinolin-3-yl)oxy)-N-methylacetamide (0.044 g) was prepared as an off-white solid in a similar manner as 2-((6-((5-chloro-2-(2-(3-(2,6-dioxopiperidin-3-yl)-1-methyl-1H-indazol-6-yl)-2,6-diazaspiro[3.5]nonan-6-yl)pyrimidin-4-yl)amino)-1-methyl-2-oxo-1,2-dihydroquinolin-3-yl)oxy)-N-methylacetamide (see, e.g., Example 76).

¹H NMR (400 MHZ, DMSO-d₆): δ 10.83 (s, 1H), 8.78 (s, 1H), 8.00-7.66 (m, 4H), 7.33-7.04 (m, 3H), 6.23 (bs, 2H), 4.28 (bs, 2H), 4.24-4.19 (m, 1H), 3.77-3.23 (m, 11H), 3.16-3.12 (m, 2H), 3.00 (bs, 1H), 2.83-2.61 (m, 5H), 2.32-2.07 (m, 2H), 1.88-1.75 (m, 1H), 1.73-1.41 (m, 5H).

LC-MS (ESI): m/z=751.41 [M−H]⁻.

### Example 79. Synthesis of 2-((6-((5-chloro-2-(4-((1-(3-(2,6-dioxopiperidin-3-yl)-1-methyl-1H-indazol-6-yl)pyrrolidin-3-yl)methyl)piperidin-1-yl)pyrimidin-4-yl)amino)-1-methyl-2-oxo-1,2-dihydroquinolin-3-yl)oxy)-N-methylacetamide (Compound 332)

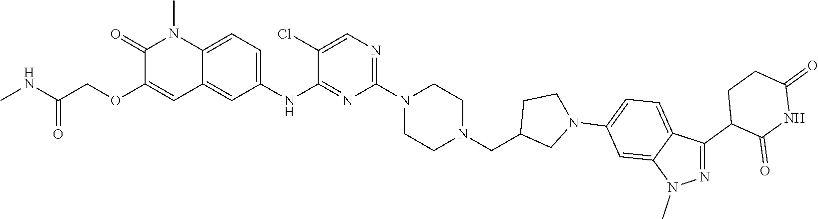 [SMILES: CNC(=O)COc1cc2cc(Nc3nc(N4CCN(CC5CCN(c6ccc7c(C8CCC(=O)NC8=O)nn(C)c7c6)C5)CC4)ncc3Cl)ccc2n(C)c1=O]

### Preparation of benzyl 4-((1-(3-(2,6-bis(benzyloxy)pyridin-3-yl)-1-methyl-1H-indazol-6-yl)pyrrolidin-3-yl)methyl)piperidine-1-carboxylate

To a stirred solution of benzyl 4-(pyrrolidin-3-ylmethyl)piperidine-1-carboxylate (1.0 g, 3.31 mmol), 3-(2,6-bis(benzyloxy)pyridin-3-yl)-6-bromo-1-methyl-1H-indazole (1.15 g, 2.32 mmol), and sodium tert-butoxide (0.64 g, 6.61 mmol) in dioxane (20 mL), RuPhos (0.15 g, 0.33 mmol) and RuPhos-Pd-G3 (0.14 g, 0.17 mmol) were added. The reaction was degassed with nitrogen for 15 minutes. The reaction was heated at 70° C. for 3 hours. Water (50 mL) was added to the reaction, and the reaction was extracted into ethyl acetate (100 mL×2). The combined organic layers were washed with brine (10 mL), dried over sodium sulfate, filtered, and concentrated under reduced pressure to obtain crude product. The crude product was purified by flash column chromatography (SiO₂, 100-200, 30-40% ethyl acetate in petroleum ether) to afford benzyl 4-((1-(3-(2,6-bis(benzyloxy)pyridin-3-yl)-1-methyl-1H-indazol-6-yl)pyrrolidin-3-yl)methyl)piperidine-1-carboxylate (0.6 g).

LC-MS (ESI): m/z=722.69 [M+H]⁺.

### Preparation of 3-(1-methyl-6-(3-(piperidin-4-ylmethyl)pyrrolidin-1-yl)-1H-indazol-3-yl)piperidine-2,6-dione

To a stirred solution of benzyl 4-((1-(3-(2,6-bis(benzyloxy)pyridin-3-yl)-1-methyl-1H-indazol-6-yl)pyrrolidin-3-yl)methyl)piperidine-1-carboxylate (0.3 g, 0.41 mmol), tetrahydrofuran (3 mL), ethyl acetate (3 mL), and acetic acid (0.6 mL), 20% palladium hydroxide on carbon (0.15 g, w/w) was added. The reaction was put under a hydrogen atmosphere (15 psi) and stirred at room temperature for 24 hours. The reaction mixture was filtered through a pad of celite. The celite was washed with THF. The filtrate was concentrated to afford 3-(1-methyl-6-(3-(piperidin-4-ylmethyl)pyrrolidin-1-yl)-1H-indazol-3-yl)piperidine-2,6-dione (0.07 g).

LC-MS (ESI): m/z=410.49 [M+H]⁺.

### Preparation of 2-((6-((5-chloro-2-(4-((1-(3-(2,6-dioxopiperidin-3-yl)-1-methyl-1H-indazol-6-yl)pyrrolidin-3-yl)methyl)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(3-(piperidin-4-ylmethyl)pyrrolidin-1-yl)-1H-indazol-3-yl)piperidine-2,6-dione (0.07 g, 0.27 mmol) in DMSO (3.6 mL), DIPEA (0.47 mL, 2.70 mmol) was added. After stirring for 10 minutes, 2-((6-((2,5-dichloropyrimidin-4-yl)amino)-1-methyl-2-oxo-1,2-dihydroquinolin-3-yl)oxy)-N-methylacetamide (0.077 g, 0.19 mmol) was added. The reaction was heated to 120° C. for 3 hours. The reaction mixture was quenched with cold water. The resulting precipitate was filtered and washed with cold water and petroleum ether to obtain crude product. The crude product was purified by prep-HPLC to afford 2-((6-((5-chloro-2-(4-((1-(3-(2,6-dioxopiperidin-3-yl)-1-methyl-1H-indazol-6-yl)pyrrolidin-3-yl)methyl)piperidin-1-yl)pyrimidin-4-yl)amino)-1-methyl-2-oxo-1,2-dihydroquinolin-3-yl)oxy)-N-methylacetamide (3.5 mg) as a grey solid.

LC-MS (ESI): m/z=781.59 [M+H]⁺.

¹H NMR (400 MHz, DMSO-d₆): 8.81 (s, 1H), 8.47 (s, 1H), 8.03 (s, 1H), 7.95-7.93 (m, 2H), 7.76-7.73 (m, 1H), 7.48-7.46 (m, 2H), 7.09 (s, 1H), 6.55-6.53 (m, 1H), 6.33 (s, 1H), 4.51 (s, 2H), 4.48-4.34 (m, 2H), 4.33-4.30 (m, 1H), 4.18 (s, 3H), 3.67 (s, 3H), 3.24-3.07 (m, 3H), 2.85-2.61 (m, 9H), 2.32 (bs, 1H), 2.18-2.15 (m, 2H), 1.75-1.73 (m, 2H), 1.59-1.54 (m, 2H), 1.42-1.40 (m, 2H), 1.23 (bs, 1H), 1.08-1.05 (m, 1H).

### Example 80. Synthesis of 2-((6-((5-chloro-2-(4-((4-(3-(2,6-dioxopiperidin-3-yl)-1-methyl-1H-indazol-7-yl)piperazin-1-yl)methyl)piperidin-1-yl)pyrimidin-4-yl)amino)-1-methyl-2-oxo-1,2-dihydroquinolin-3-yl)oxy)-N-methylacetamide (Compound 333)

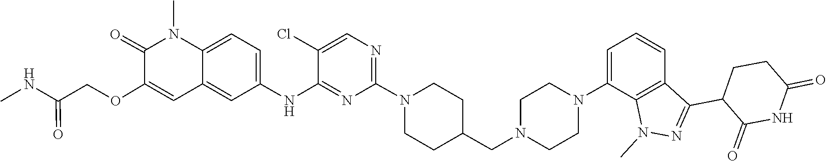 [SMILES: CNC(=O)COc1cc2cc(Nc3nc(N4CCC(CN5CCN(c6cccc7c(C8CCC(=O)NC8=O)nn(C)c67)CC5)CC4)ncc3Cl)ccc2n(C)c1=O]

### Preparation of benzyl 4-((1-(tert-butoxycarbonyl)piperidin-4-yl)methyl)piperazine-1-carboxylate

To a solution of benzyl piperazine-1-carboxylate (10.0 g, 45.4 mmol), tert-butyl 4-formylpiperidine-1-carboxylate (11.6 g, 54.5 mmol), and NaBH(OAc)₃ (9.62 g, 45.4 mmol) in DCM (200 mL) was added acetic acid (2.72 g, 45.4 mmol). The reaction was stirred at room temperature for 16 hours. The reaction mixture was quenched with ice-cold water (500 mL) and extracted into DCM (3×250 mL). The combined organic layers were washed with brine solution (500 mL) and dried over anhydrous Na₂SO₄, filtered, and dried under vacuum to get crude product. The crude product was purified by column chromatography (SiO₂; 40% EtOAc in petroleum ether) to afford benzyl 4-((1-(tert-butoxycarbonyl)piperidin-4-yl)methyl)piperazine-1-carboxylate (6.0 g) as colorless liquid.

LC-MS (ESI): m/z=418.31 [M+H]⁺.

### Preparation of tert-butyl 4-(piperazin-1-ylmethyl)piperidine-1-carboxylate

To a stirred solution of benzyl 4-((1-(tert-butoxycarbonyl)piperidin-4-yl)methyl)piperazine-1-carboxylate (6.0 g, 14.4 mmol) in THF (180 mL) was added 20% Pd(OH)₂ on carbon (3.0 g). The reaction was put under a hydrogen atmosphere (60 psi) at room temperature and stirred for 16 hours. The reaction mixture was filtered through a celite bed and concentrated to obtain the crude product. The crude product was washed with n-pentane and dried to afford tert-butyl 4-(piperazin-1-ylmethyl)piperidine-1-carboxylate (3.0 g) as colorless liquid.

LC-MS (ESI): m/z=284.16 [M+H]⁺.

### Preparation of tert-butyl 4 ((4-(3-(2,6-bis(benzyloxy)pyridin-3-yl)-1-methyl-1H-indazol-7-yl)piperazin-1-yl)methyl)piperidine-1-carboxylate

A solution of 3-(2,6-bis(benzyloxy)pyridin-3-yl)-7-bromo-1-methyl-1H-indazole (1.0 g, 2.0 mmol), tert-butyl 4-(piperazin-1-ylmethyl)piperidine-1-carboxylate (0.56 g, 2.0 mmol) and cesium carbonate (1.95 g, 6.0 mmol) in 1,4-dioxane (20 mL) was degassed with argon for 5 minutes. Pd-PEPPSI-IHeptCl (0.97 g, 0.1 mmol) was added to the reaction, and the reaction was again degassed for 5 minutes. The reaction mixture was heated at 80° C. for 16 hours. The reaction mixture was quenched with ice-cold water (50 mL) and extracted into ethyl acetate (3×100 mL). The combined organic layers were washed with brine solution (50 mL), dried over anhydrous Na₂SO₄, filtered, and dried under vacuum to get crude product. The crude product was purified by column chromatography (SiO₂; 40% EtOAc in petroleum ether) to afford tert-butyl 4-((4-(3-(2,6-bis(benzyloxy)pyridin-3-yl)-1-methyl-1H-indazol-7-yl)piperazin-1-yl)methyl)piperidine-1-carboxylate (1.0 g) as pale brown semi solid.

LC-MS (ESI): m/z=703.80 [M+H]⁺.

### Preparation of tert-butyl 4-((4-(3-(2,6-dioxopiperidin-3-yl)-1-methyl-1H-indazol-7-yl)piperazin-1-yl)methyl)piperidine-1-carboxylate

To a stirred solution of tert-butyl 4-((4-(3-(2,6-bis(benzyloxy)pyridin-3-yl)-1-methyl-1H-indazol-7-yl)piperazin-1-yl)methyl)piperidine-1-carboxylate (1.0 g, 1.42 mmol) in THF (60 mL) was added 20% Pd(OH)₂ on carbon (1.0 g). The reaction was put under hydrogen atmosphere (60 psi) and stirred at room temperature for 6 hours. The reaction mixture was filtered through a celite bed and concentrated to obtain crude product. The crude product was washed with n-pentane and dried to afford tert-butyl 4-((4-(3-(2,6-dioxopiperidin-3-yl)-1-methyl-1H-indazol-7-yl)piperazin-1-yl)methyl)piperidine-1-carboxylate (0.70 g) as brown solid.

LC-MS (ESI): m/z=525.61 [M+H]⁺.

### Preparation of 3-(1-methyl-7-(4-(piperidin-4-ylmethyl)piperazin-1-yl)-1H-indazol-3-yl)piperidine-2,6-dione

A stirred solution of tert-butyl 4-((4-(3-(2,6-dioxopiperidin-3-yl)-1-methyl-1H-indazol-7-yl)piperazin-1-yl)methyl)piperidine-1-carboxylate (0.70 g, 1.33 mmol) in DCM (14 mL) was cooled to 0° C., and TFA (7.0 mL) was added. The reaction was allowed to warm to room temperature over 3 hours. The reaction mixture was concentrated to obtain crude product. The crude product was washed with n-pentane to obtain 3-(1-methyl-7-(4-(piperidin-4-ylmethyl)piperazin-1-yl)-1H-indazol-3-yl)piperidine-2,6-dione (0.50 g) as a brown gummy solid.

LC-MS (ESI): m/z=425.51 [M+H]⁺.

### Preparation of 2-((6-((5-chloro-2-(4-((4-(3-(2,6-dioxopiperidin-3-yl)-1-methyl-1H-indazol-7-yl)piperazin-1-yl)methyl)piperidin-1-yl)pyrimidin-4-yl)amino)-1-methyl-2-oxo-1,2-dihydroquinolin-3-yl)oxy)-N-methylacetamide

To a stirred solution 3-(1-methyl-7-(4-(piperidin-4-ylmethyl)piperazin-1-yl)-1H-indazol-3-yl)piperidine-2,6-dione (0.15 g, 0.35 mmol) and 2-((6-((2,5-dichloropyrimidin-4-yl)amino)-1-methyl-2-oxo-1,2-dihydroquinolin-3-yl)oxy)-N-methylacetamide (0.11 g, 0.28 mmol) in DMSO, N,N-diisopropylethylamine (0.36 g, 2.82 mmol) was added. The reaction mixture was stirred at 100° C. for 16 hours. The reaction mixture was poured into ice-cold water and stirred for 15 minutes. The resulting precipitate was filtered and dried to obtain crude product. The crude product was purified by prep-HPLC to afford 2-((6-((5-chloro-2-(4-((4-(3-(2,6-dioxopiperidin-3-yl)-1-methyl-1H-indazol-7-yl)piperazin-1-yl)methyl)piperidin-1-yl)pyrimidin-4-yl)amino)-1-methyl-2-oxo-1,2-dihydroquinolin-3-yl)oxy)-N-methylacetamide (0.052 g) as an off-white solid.

LC-MS (ESI): m/z=796.46 [M+H]⁺.

¹H NMR (400 MHZ, DMSO-d₆): δ 10.88 (s, 1H), 8.82 (s, 1H), 8.04 (s, 1H), 7.94-7.89 (m, 2H), 7.78-7.76 (m, 1H), 7.48 (d, J=9.20 Hz, 1H), 7.40-7.38 (m, 1H), 7.12 (s, 1H), 7.03-7.02 (m, 1H), 4.58-4.49 (m, 4H), 4.33 (q, J=5.20 Hz, 1H), 4.24 (s, 3H), 3.68 (s, 3H), 3.21-2.82 (m, 8H), 2.67-2.59 (m, 5H), 2.42-2.14 (m, 5H), 1.89-1.76 (m, 4H), 1.07-1.04 (m, 2H).

### Example 81. Synthesis of 2-((6-((5-chloro-2-(4-((4-(3-(2,6-dioxopiperidin-3-yl)-1-methyl-1H-indazol-6-yl)piperazin-1-yl)methyl)piperidin-1-yl)pyrimidin-4-yl)amino)-1-methyl-2-oxo-1,2-dihydroquinolin-3-yl)oxy)-N-methylacetamide (Compound 334)

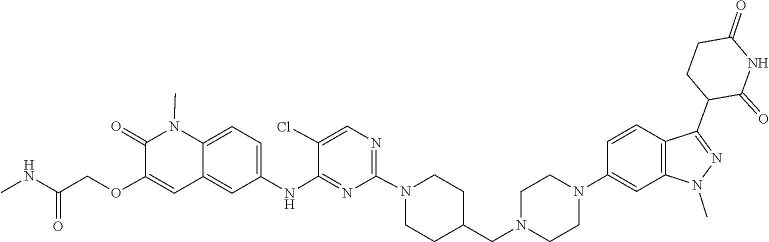 [SMILES: CNC(=O)COc1cc2cc(Nc3nc(N4CCC(CN5CCN(c6ccc7c(C8CCC(=O)NC8=O)nn(C)c7c6)CC5)CC4)ncc3Cl)ccc2n(C)c1=O]

### Preparation of tert-butyl 4-((4-(3-(2,6-bis(benzyloxy)pyridin-3-yl)-1-methyl-1H-indazol-6-yl)piperazin-1-yl)methyl)piperidine-1-carboxylate

A solution of 3-(2,6-bis(benzyloxy)pyridin-3-yl)-6-bromo-1-methyl-1H-indazole (0.8 g, 1.59 mmol), tert-butyl 4-(piperazin-1-ylmethyl)piperidine-1-carboxylate (0.9 g, 2.0 mmol), and cesium carbonate (1.55 g, 4.77 mmol) in 1,4-dioxane (16 mL) was degassed with argon for 5 minutes. Ruphos-Pd-G3 (0.066 g, 0.079 mmol) and Ruphos (0.069 g, 0.15 mmol) was added. The reaction was degassed for 5 minutes and then heated at 100° C. for 16 hours. The reaction mixture was quenched with ice-cold water (40 mL) and extracted into ethyl acetate (3×100 mL). The combined organic layers were washed with brine solution (40 mL), dried over anhydrous Na₂SO₄, filtered, and dried under vacuum to get crude product. The crude product was purified by column chromatography (SiO₂; 50% EtOAc in petroleum ether) to afford tert-butyl 4-((4-(3-(2,6-bis(benzyloxy)pyridin-3-yl)-1-methyl-1H-indazol-6-yl)piperazin-1-yl)methyl)piperidine-1-carboxylate (0.8 g) as pale brown semi solid.

LC-MS (ESI): m/z=703.80. [M+H]⁺.

### Preparation of tert-butyl 4-((4-(3-(2,6-dioxopiperidin-3-yl)-1-methyl-1H-indazol-6-yl)piperazin-1-yl)methyl)piperidine-1-carboxylate

To a stirred solution of tert-butyl 4-((4-(3-(2,6-bis(benzyloxy)pyridin-3-yl)-1-methyl-1H-indazol-6-yl)piperazin-1-yl)methyl)piperidine-1-carboxylate (1.0 g, 1.13 mmol) in THF (40 mL) was added 20% Pd(OH)₂ on carbon (0.8 g). The reaction was stirred under hydrogen atmosphere (60 psi) at room temperature for 6 hours. The reaction mixture was filtered through a celite bed, the celite was washed with THE, and the filtrate was concentrated. The crude product was washed with n-pentane and dried to afford tert-butyl 4-((4-(3-(2,6-dioxopiperidin-3-yl)-1-methyl-1H-indazol-6-yl)piperazin-1-yl)methyl)piperidine-1-carboxylate (0.5 g) as a brown solid.

LC-MS (ESI): m/z=525.66 [M+H]⁺.

### Preparation of 3-(1-methyl-6-(4-(piperidin-4-ylmethyl)piperazin-1-yl)-1H-indazol-3-yl)piperidine-2,6-dione

A stirred solution of tert-butyl 4-((4-(3-(2,6-dioxopiperidin-3-yl)-1-methyl-1H-indazol-6-yl)piperazin-1-yl)methyl)piperidine-1-carboxylate (0.5 g, 0.95 mmol) in DCM (10 mL) was cooled to 0° C. and TFA was added (5.0 mL). The reaction mixture was stirred at room temperature for 3 hours. The reaction mixture was concentrated to obtain crude product. The crude product was washed with n-pentane to afford 3-(1-methyl-6-(4-(piperidin-4-ylmethyl)piperazin-1-yl)-1H-indazol-3-yl)piperidine-2,6-dione (0.350 g) as a brown gummy solid.

LC-MS (ESI): m/z=425.52 [M+H]⁺.

### Preparation of 2-((6-((5-chloro-2-(4-((4-(3-(2,6-dioxopiperidin-3-yl)-1-methyl-1H-indazol-6-yl)piperazin-1-yl)methyl)piperidin-1-yl)pyrimidin-4-yl)amino)-1-methyl-2-oxo-1,2-dihydroquinolin-3-yl)oxy)-N-methylacetamide

To a stirred solution 3-(1-methyl-6-(4-(piperidin-4-ylmethyl)piperazin-1-yl)-1H-indazol-3-yl)piperidine-2,6-dione (0.15 g, 0.35 mmol) and 2-((6-((2,5-dichloropyrimidin-4-yl)amino)-1-methyl-2-oxo-1,2-dihydroquinolin-3-yl)oxy)-N-methylacetamide (0.11 g, 0.28 mmol) in DMSO (3 mL), N,N-diisopropylethylamine (0.36 g, 2.82 mmol) was added. The reaction mixture was stirred at 100° C. for 16 hours. The reaction mixture was filtered, and solvent was removed using a centrifugal evaporator to obtain crude product. The crude product was purified by prep-HPLC to afford 2-((6-((5-chloro-2-(4-((4-(3-(2,6-dioxopiperidin-3-yl)-1-methyl-1H-indazol-6-yl)piperazin-1-yl)methyl)piperidin-1-yl)pyrimidin-4-yl)amino)-1-methyl-2-oxo-1,2-dihydroquinolin-3-yl)oxy)-N-methylacetamide (24 mg) as a brown solid.

LC-MS (ESI): m/z=796.46 [M+H]⁺.

¹H NMR (400 MHZ, DMSO-d₆): δ 10.85 (s, 1H), 8.81 (s, 1H), 8.04 (s, 1H), 7.95-7.92 (m, 2H), 7.77 (dd, J=2.00, 9.00 Hz, 1H), 7.50-7.47 (m, 2H), 7.11 (s, 1H), 6.91 (d, J=9.20 Hz, 1H), 6.83 (s, 1H), 4.57-4.48 (m, 4H), 4.27-4.23 (m, 1H), 3.89 (s, 3H), 3.67 (s, 3H), 3.21 (br s, 4H), 2.86 (t, J=12.00 Hz, 2H), 2.88-2.59 (m, 9H), 2.29-2.28 (m, 2H), 2.13-2.07 (m, 2H), 1.90-1.78 (m, 3H), 1.24 (m, 2H).

### Example 82. Synthesis of 2-((6-((5-chloro-2-(8-(3-(2,6-dioxopiperidin-3-yl)-1-methyl-1H-indazol-6-yl)-2,8-diazaspiro[5.5]undecan-2-yl)pyrimidin-4-yl)amino)-1-methyl-2-oxo-1,2-dihydroquinolin-3-yl)oxy)-N-methylacetamide (Compound 335)

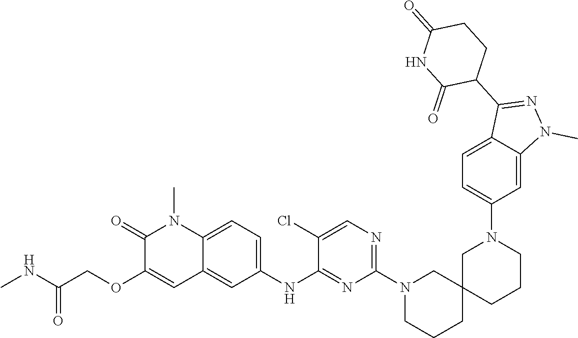

### Preparation of 2-((6-((5-chloro-2-(8-(3-(2,6-dioxopiperidin-3-yl)-1-methyl-1H-indazol-6-yl)-2,8-diazaspiro[5.5]undecan-2-yl)pyrimidin-4-yl)amino)-1-methyl-2-oxo-1,2-dihydroquinolin-3-yl)oxy)-N-methylacetamide

2-((6-((5-chloro-2-(8-(3-(2,6-dioxopiperidin-3-yl)-1-methyl-1H-indazol-6-yl)-2,8-diazaspiro[5.5]undecan-2-yl)pyrimidin-4-yl)amino)-1-methyl-2-oxo-1,2-dihydroquinolin-3-yl)oxy)-N-methylacetamide (0.074 g) was prepared as a pale pink solid in a similar manner as 2-((6-((5-chloro-2-(2-(3-(2,6-dioxopiperidin-3-yl)-1-methyl-1H-indazol-6-yl)-2,6-diazaspiro[3.5]nonan-6-yl)pyrimidin-4-yl)amino)-1-methyl-2-oxo-1,2-dihydroquinolin-3-yl)oxy)-N-methylacetamide (see, e.g., Example 76).

LC-MS (ESI): m/z=767.48 [M+H]⁺.

¹H NMR (400 MHZ, DMSO-d₆): δ 10.90 (s, 1H), 8.74 (s, 1H), 8.12-7.36 (m, 5H), 7.07 (s, 1H), 6.92-6.45 (m, 2H), 4.49 (s, 2H), 4.24-4.22 (m, 1H), 3.92-3.45 (m, 11H), 3.20 (br, 4H), 2.62-2.61 (m, 5H), 2.40-2.20 (m, 1H), 2.20-2.10 (m, 1H), 1.56-1.40 (m, 7H), 1.10 (s, 1H).

### Example 83. Synthesis of 2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide

[rel-2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide] (Compound 307a)

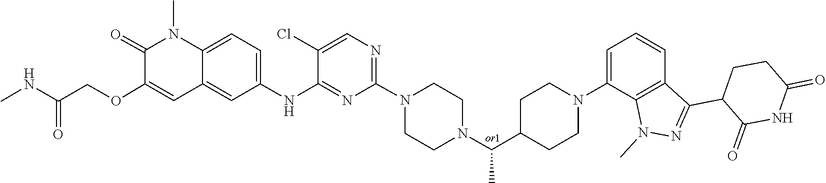 [SMILES: CNC(=O)COc1cc2cc(Nc3nc(N4CCN([C@@H](C)C5CCN(c6cccc7c(C8CCC(=O)NC8=O)nn(C)c67)CC5)CC4)ncc3Cl)ccc2n(C)c1=O]

### Preparation of benzyl 4-acetylpiperidine-1-carboxylate

A stirred solution of 1-(piperidin-4-yl) ethan-1-one hydrochloride (10.0 g, 61.3 mmol) in DCM (200 mL) was cooled to 0° C. under N₂ atmosphere. TEA (25.6 mL, 184 mmol) was added, and the reaction was for 5 minutes. Benzyl chloroformate (13.1 mL, 92.0 mmol) was added and the resultant reaction mixture was allowed to warm to room temperature over 16 hours while stirring. The reaction mixture was quenched with cold water and extracted in DCM. The combined organic layers were dried over anhydrous sodium sulfate, filtered, and concentrated to get crude product. The crude product was purified by flash column chromatography (SiO₂, 100-200, 25% ethyl acetate in petroleum ether) to afford benzyl 4-acetylpiperidine-1-carboxylate as a white solid (13.0 g).

LC-MS (ESI): m/z=284.31 [M+Na]⁺.

### Preparation of tert-butyl 4-(1-(1-((benzyloxy)carbonyl)piperidin-4-yl)ethyl)piperazine-1-carboxylate

A stirred solution of benzyl 4-acetylpiperidine-1-carboxylate (10 g, 38.3 mmol) and tert-butyl piperazine-1-carboxylate (14.3 g, 76.5 mmol) in ethanol (200 mL) was cooled to 0° C. under N₂ atmosphere. Titanium (IV) isopropoxide (10 mL) was added, and the reaction was allowed to warm to room temperature over 2 hours under N₂ atmosphere. Sodium cyanoborohydride (7.21 g, 114 mmol) was added, and the reaction mixture was heated to 50° C. for 16 hours. The reaction mixture was quenched with cold water and filtered through a celite bed and the celite was washed with EtOAc. The combined organic layers were washed with brine, dried over anhydrous sodium sulfate, filtered, and concentrated to get crude product. The crude product was purified by flash column chromatography (SiO₂, 100-200, 10% ethyl acetate in petroleum ether) to afford tert-butyl 4-(1-(1-((benzyloxy)carbonyl)piperidin-4-yl)ethyl)piperazine-1-carboxylate as a white solid (13.0 g).

LC-MS (ESI): m/z=432.50 [M+H]⁺.

The tert-butyl 4-(1-(1-((benzyloxy)carbonyl)piperidin-4-yl)ethyl)piperazine-1-carboxylate enantiomers were separated via chiral HPLC SFC purification (Column/dimensions: Chiralcel OX-H (4.6 mm×250 mm, internal diameter 5 μm); mobile phase: Phase A (CO₂) and Phase B (MeOH with 0.5% diethylamine); isocratic elution B %: 20%; flow rate: 3.0 mL/min; detector: PDA; wavelength: 220 nm; column temperature: 30° C.; back pressure: 100 bar) to afford tert-butyl 4-(1-(1-((benzyloxy)carbonyl)piperidin-4-yl)ethyl)piperazine-1-carboxylate (Isomer 1, Rt=5.77 min)(3.6 g) and tert-butyl 4-(1-(1-((benzyloxy)carbonyl)piperidin-4-yl)ethyl)piperazine-1-carboxylate (Isomer 2, Rt=6.67 min)(3.5 g).

### Preparation of tert-butyl 4-(1-(piperidin-4-yl)ethyl)piperazine-1-carboxylate (Isomer 1)

A stirred solution of tert-butyl 4-(1-(1-((benzyloxy)carbonyl)piperidin-4-yl)ethyl)piperazine-1-carboxylate (Isomer 1) (1.5 g, 3.47 mmol) in THF (30 mL) was degassed with nitrogen for 5 minutes. 10% Pd/C (0.37 g, 0.37 mmol) was added and the reaction was put under hydrogen atmosphere (50 psi) and stirred at room temperature for 16 hours. The reaction mixture was diluted with THF (100 mL) and filtered through a pad of celite. The filtrate was collected and concentrated to afford tert-butyl 4-(1-(piperidin-4-yl)ethyl)piperazine-1-carboxylate (Isomer 1) (1.0 g) as a brown semi-solid.

LC-MS (ESI): m/z=298.24 [M+H]⁺.

### Preparation of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 1)

A stirred solution of 3-(2,6-bis(benzyloxy)pyridin-3-yl)-7-bromo-1-methyl-1H-indazole (1.4 g, 2.8 mmol) and tert-butyl 4-(1-(piperidin-4-yl)ethyl)piperazine-1-carboxylate (Isomer 1) (1.0 g, 3.3 mmol) in 1,4-dioxane (28 mL) was put under N₂ atmosphere. Cesium carbonate (1.82 g, 5.6 mmol) was added and the reaction was degassed with N₂ for 10 minutes. PEPPSI-iHept-C₁ (0.13 g, 0.14 mmol) was added and the reaction was stirred at 100° C. for 3 hours. The reaction mixture was filtered through a celite bed and the celite was washed with EtOAc. The combined organic layers were concentrated and purified by flash column chromatography (SiO₂, 100-200, 27% ethyl acetate in petroleum ether) to afford tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 1) (2.0 g).

LC-MS (ESI): m/z=717.25 [M+H]⁺.

### Preparation of tert-butyl 4-(1-(1-(3-(2,6-dioxopiperidin-3-yl)-1-methyl-1H-indazol-7-yl)piperidin-4-yl)ethyl)piperazine-1-carboxylate (Isomer 1)

A stirred solution of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 1) (1.0 g, 1.39 mmol) in THF (50 mL) was degassed with nitrogen for 5 minutes. 20% Pd(OH)₂/C (1.0 g, 1.39 mmol) was added, and the reaction was put under hydrogen atmosphere with balloon pressure at room temperature for 16 hours. The reaction mixture was diluted with ethyl acetate (350 mL) and filtered through a pad of celite. The filtrate was collected and concentrated to afford tert-butyl 4-(1-(1-(3-(2,6-dioxopiperidin-3-yl)-1-methyl-1H-indazol-7-yl)piperidin-4-yl)ethyl)piperazine-1-carboxylate (Isomer 1) (0.6 g).

LC-MS (ESI): m/z=539.69 [M+H]⁺.

### Preparation of 3-(1-methyl-7-(4-(1-(piperazin-1-yl)ethyl)piperidin-1-yl)-1H-indazol-3-yl)piperidine-2,6-dione hydrochloride (Isomer 1)

To a stirred solution of tert-butyl 4-(1-(1-(3-(2,6-dioxopiperidin-3-yl)-1-methyl-1H-indazol-7-yl)piperidin-4-yl)ethyl)piperazine-1-carboxylate (Isomer 1) (0.6 g, 1.11 mmol) in 1,4-dioxane (3 mL) was added 4 M HCl in 1,4-dioxane (3 mL) at 0° C. under a N₂ atmosphere. The reaction was stirred at room temperature for 3 hours. The reaction mixture was concentrated under reduced pressure to remove solvents and then co-distilled with DCM to afford 3-(1-methyl-7-(4-(1-(piperazin-1-yl)ethyl)piperidin-1-yl)-1H-indazol-3-yl)piperidine-2,6-dione hydrochloride (Isomer 1) (0.40 g).

LC-MS (ESI): m/z=439.57 [M+H]⁺.

### Preparation of 2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide

[rel-2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide]

A stirred solution of 3-(1-methyl-7-(4-(1-(piperazin-1-yl)ethyl)piperidin-1-yl)-1H-indazol-3-yl)piperidine-2,6-dione hydrochloride (Isomer 1) (0.2 g, 0.42 mmol) in DMSO (4.0 mL) was put under nitrogen atmosphere. DIPEA (0.36 mL, 2.10 mmol) was added, and the reaction was stirred at room temperature for 5 minutes. 2-((6-((2,5-dichloropyrimidin-4-yl)amino)-1-methyl-2-oxo-1,2-dihydroquinolin-3-yl)oxy)-N-methylacetamide (0.12 g, 0.29 mmol) was added, and the reaction mixture was heated to 100° C. for 3 hours. The reaction mixture was purified by prep-HPLC to afford 2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide [rel-2-((6-((5-chloro-2-(4-((1S)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide] (0.042 g) as a brown solid.

LC-MS (ESI): m/z=810.51 [M+H]⁺.

¹H NMR (400 MHZ, DMSO-d₆): δ 10.9 (s, 1H), 8.84 (s, 1H), 8.17 (s, 1H), 8.04 (s, 1H), 7.96-7.93 (m, 2H), 7.76-7.76 (m, 1H), 7.37-7.35 (m, 1H), 7.12 (s, 1H), 7.01-6.99 (d, 2H, J=8 Hz), 4.58 (s, 2H), 4.34-4.30 (m, 1H), 4.34 (s, 3H), 3.67-3.64 (d, 7H J=12 Hz), 2.32 (s, 2H), 2.67-2.60 (m, 8H), 2.37-2.32 (m, 5H), 2.18-2.17 (d, 2H, J-4 Hz), 1.83-1.80 (d, 1H, J=12 Hz), 1.53 (s, 1H), 1.39-1.37 (d, 2H, J=8 Hz), 0.92-0.91 (d, 3H, J=4 Hz).

### Example 84. Synthesis of 2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide

[rel-2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide] (Compound 307b)

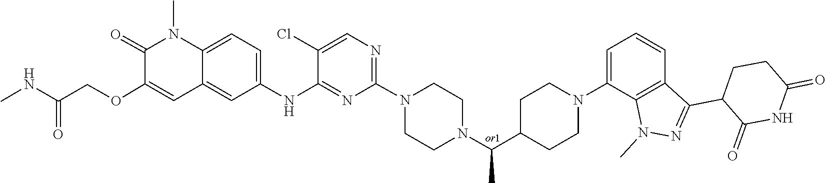 [SMILES: CNC(=O)COc1cc2cc(Nc3nc(N4CCN([C@H](C)C5CCN(c6cccc7c(C8CCC(=O)NC8=O)nn(C)c67)CC5)CC4)ncc3Cl)ccc2n(C)c1=O]

### Preparation of tert-butyl 4-(1-(piperidin-4-yl)ethyl)piperazine-1-carboxylate (Isomer 2)

A stirred solution of tert-butyl 4-(1-(1-((benzyloxy)carbonyl)piperidin-4-yl)ethyl)piperazine-1-carboxylate (Isomer 2) (1.5 g, 3.47 mmol) in tetrahydrofuran (30 mL) was put under N₂ atmosphere. Palladium on carbon, 10% Pd (Dry)(0.3 g, 20% w/w) was added, and the resultant reaction mixture was stirred under hydrogen pressure (50 psi) at room temperature for 16 hours. The reaction mixture was filtered through a celite bed and the celite was washed with EtOAc and THF. The combined organic layers were concentrated to obtain tert-butyl 4-(1-(piperidin-4-yl)ethyl)piperazine-1-carboxylate (Isomer 2) as a brown sticky solid (1.0 g).

LC-MS (ESI): m/z=298.20 [M+H]⁺.

### Preparation of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 2)

A stirred solution of 3-(2,6-bis(benzyloxy)pyridin-3-yl)-7-bromo-1-methyl-1H-indazole (1.4 g, 2.80 mmol) and tert-butyl 4-(1-(piperidin-4-yl)ethyl)piperazine-1-carboxylate (Isomer 2) (1.0 g, 3.35 mmol) in 1,4-dioxane (28 mL) was put under N₂ atmosphere. Cesium carbonate (2.73 g, 8.39 mmol) was added and the reaction was degassed with N₂ for 10 minutes. Pd-PEPPSI-iHEPT-Cl (0.14 g, 0.14 mmol) was added and the resultant reaction mixture was heated to 100° C. for 16 hours. The reaction was filtered through a celite bed and the celite was washed with EtOAc. The combined organic layers were washed with cold brine, dried over anhydrous sodium sulfate, filtered, and concentrated to afford crude product. The crude product was purified by flash column chromatography (SiO₂, 100-200, 18% ethyl acetate in petroleum ether) to afford tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 2) (1.4 g) as an off white solid.

LC-MS (ESI): m/z=717.97 [M+H]⁺.

### Preparation of tert-butyl 4-(1-(1-(3-(2,6-dioxopiperidin-3-yl)-1-methyl-1H-indazol-7-yl)piperidin-4-yl)ethyl)piperazine-1-carboxylate (Isomer 2)

A stirred solution of tert-butyl 4-(1-(1-(3-(2,6-bis(benzyloxy)pyridin-3-yl)-1-methyl-1H-indazol-7-yl)piperidin-4-yl)ethyl)piperazine-1-carboxylate (Isomer 2) (1.0 g, 1.40 mmol) in tetrahydrofuran (40 mL) was put under N₂ atmosphere. Palladium hydroxide on carbon, 20% Pd (0.50 g, 50% w/w) was added, and the reaction was stirred under hydrogen atmosphere with balloon pressure at room temperature for 16 hours. The reaction was filtered through a celite bed and the celite was washed with EtOAc. The combined organic layers and concentrated to obtain crude product. The crude product was purified by triturating with n-hexane to afford tert-butyl 4-(1-(1-(3-(2,6-dioxopiperidin-3-yl)-1-methyl-1H-indazol-7-yl)piperidin-4-yl)ethyl)piperazine-1-carboxylate (Isomer 2) (0.7 g) as an off white solid.

LC-MS (ESI): m/z=539.63 [M+H]⁺.

### Preparation of 3-(1-methyl-7-(4-(1-(piperazin-1-yl)ethyl)piperidin-1-yl)-1H-indazol-3-yl)piperidine-2,6-dione hydrochloride (Isomer 2)

To a stirred solution tert-butyl 4-(1-(1-(3-(2,6-dioxopiperidin-3-yl)-1-methyl-1H-indazol-7-yl)piperidin-4-yl)ethyl)piperazine-1-carboxylate (Isomer 2) (0.70 g, 1.30 mmol) in DCM (7.0 mL) was added 4 M HCl in dioxane (3.50 mL) at 0° C. under N₂ atmosphere. The reaction was stirred at room temperature for 5 hours. The reaction was concentrated under reduced pressure and co-distilled with DCM to obtain crude product. The crude product was purified by triturating with diethyl ether to afford 3-(1-methyl-7-(4-(1-(piperazin-1-yl)ethyl)piperidin-1-yl)-1H-indazol-3-yl)piperidine-2,6-dione hydrochloride (Isomer 2) (0.84 g) as a white solid.

LC-MS (ESI): m/z=439.65 [M+H]⁺.

### Preparation of 2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide

[rel-2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide]

A stirred solution of 3-(1-methyl-7-(4-(1-(piperazin-1-yl)ethyl)piperidin-1-yl)-1H-indazol-3-yl)piperidine-2,6-dione hydrochloride (Isomer 2) (0.20 g, 0.42 mmol) in DMSO (4.0 mL) was cooled to 0° C. and put under a nitrogen atmosphere. DIPEA (0.59 mL, 3.40 mmol) was added, and the reaction mixture was allowed to warm room temperature for 5 minutes under nitrogen atmosphere. 2-((6-((2,5-dichloropyrimidin-4-yl)amino)-1-methyl-2-oxo-1,2-dihydroquinolin-3-yl)oxy)-N-methylacetamide (0.086 g, 0.21 mmol) was added, and the reaction was heated to 100° C. for 7 hours. The reaction mixture was purified by prep-HPLC to afford 2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide [rel-2-((6-((5-chloro-2-(4-((1R)-1-(1-(3-(2,6-dioxopiperidin-3-yl)-1-methyl-1H-indazol-7-yl)piperidin-4-yl)ethyl)piperazin-1-yl)pyrimidin-4-yl)amino)-1-methyl-2-oxo-1,2-dihydroquinolin-3-yl)oxy)-N-methylacetamide] (0.068 g) as an off-white solid.

LC-MS (ESI): m/z=808.42 [M−H]⁺.

¹H NMR (400 MHZ, DMSO-d₆): δ 10.84 (s, 1H), 8.84 (s, 1H), 8.04 (s, 1H), 7.96-7.93 (m, 2H), 7.76-7.73 (dd, J=9.2 Hz, J=2.4 Hz, 1H), 7.49-7.46 (d, J=9.2 Hz, 1H), 7.37-7.35 (dd, J=6.8 Hz, j=2.0 Hz, 1H), 7.12 (s, 1H), 7.01-6.99 (m, 2H), 4.58 (s, 2H), 4.34-4.30 (m, 1H), 4.24 (s, 3H), 3.67-3.64 (m, 7H), 3.32 (s, 2H), 2.67-2.60 (m, 9H), 2.37-2.33 (m, 4H), 2.18-2.14 (m, 2H), 1.83-1.80 (m, 1H), 1.53 (m, 1H), 1.39-1.37 (m, 2H), 0.92-0.91 (d, J=6.4 Hz, 3H).

### Example 85. Synthesis of 2-((6-((5-chloro-2-(4-((4-(3-(2,6-dioxopiperidin-3-yl)benzoyl)piperazin-1-yl)methyl)piperidin-1-yl)pyrimidin-4-yl)amino)-1-methyl-2-oxo-1,2-dihydroquinolin-3-yl)oxy)-N-methylacetamide (Compound 314)

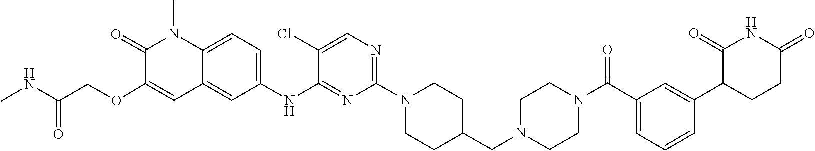 [SMILES: CNC(=O)COc1cc2cc(Nc3nc(N4CCC(CN5CCN(C(=O)c6cccc(C7CCC(=O)NC7=O)c6)CC5)CC4)ncc3Cl)ccc2n(C)c1=O]

### Preparation of 3-(2,6-bis(benzyloxy)pyridin-3-yl)benzoic acid

Sodium carbonate (791 mg, 7.46 mmol), 3-bromobenzoic acid (500 mg, 2.49 mmol), (2,6-bis(benzyloxy)pyridin-3-yl) boronic acid (1.00 g, 2.98 mmol), 1,4-dioxane (22 mL), and water (3 mL) were combined. The reaction was flushed with nitrogen and methanesulfonato (2-dicyclohexylphosphino-2′,4′,6′-tri-i-propyl-1,1′-biphenyl)(2′-methylamino-1,1′-biphenyl-2-yl)palladium (II)(42.9 mg, 49.7 μmol) was added. The reaction was heated to 90° C. for 6 hours. The reaction was cooled and diluted with EtOAc and 1 N HCl. The aqueous layer was extracted 2× with EtOAc. The combined organic layers were dried with MgSO₄, filtered, and loaded onto silica. The crude product was purified via flash column chromatography to afford 3-(2,6-bis(benzyloxy)pyridin-3-yl)benzoic acid (915 mg).

LC-MS (ESI): m/z=412.2 [M+H]⁺.

### Preparation of 3-(2,6-dioxopiperidin-3-yl)benzoic acid

3-(2,6-Bis(benzyloxy)pyridin-3-yl)benzoic acid (915 mg, 2.22 mmol) was dissolved in EtOAc (30 mL). The reaction was flushed with nitrogen. Palladium on carbon (237 mg, 10% w/v) was added, and the reaction was put under a hydrogen atmosphere (balloon pressure) and stirred at 50° C. for 12 hours. Additional EtOAc (200 mL) and hydrogen (balloon pressure) were added. The reaction was stirred for an additional 48 hours at 50° C. The reaction was filtered through a pad of celite and concentrated to afford 3-(2,6-dioxopiperidin-3-yl)benzoic acid (540 mg).

LC-MS (ESI): m/z=234.0 [M+H]⁺.

### Preparation of tert-butyl 4-((4-(3-(2,6-dioxopiperidin-3-yl)benzoyl)piperazin-1-yl)methyl)piperidine-1-carboxylate

A mixture of tert-butyl 4-(piperazin-1-ylmethyl)piperidine-1-carboxylate (150 mg, 529 μmol), 3-(2,6-dioxopiperidin-3-yl)benzoic acid (148 mg, 635 μmol), and NMI (88.6 μL, 1.11 mmol) in MeCN (3.00 mL) was treated with TCFH (178 mg, 635 μmol) and MeCN (1.00 mL). The reaction mixture was allowed to stir for 1 hour, then diluted with DMSO, concentrated in vacuo to remove acetonitrile, filtered, and purified by reverse phase ISCO (20-50% MeCN—H2O [+0.1% FA]) to afford tert-butyl 4-((4-(3-(2,6-dioxopiperidin-3-yl)benzoyl)piperazin-1-yl)methyl)piperidine-1-carboxylate (127 mg).

LC-MS (ESI): m/z=499.6 [M+H]⁺.

### Preparation of 3-(3-(4-(piperidin-4-ylmethyl)piperazine-1-carbonyl)phenyl)piperidine-2,6-dione dihydrochloride

A mixture of tert-butyl 4-((4-(3-(2,6-dioxopiperidin-3-yl)benzoyl)piperazin-1-yl)methyl)piperidine-1-carboxylate (124 mg, 248 μmol) and DCM (1.00 mL) was treated with HCl (4.0 M in dioxane)(1.00 mL, 4.00 mmol) and stirred at room temperature for 21 hour. The volatiles were removed under reduced pressure. The residue was suspended in DCM and concentrated twice more under reduced pressure to afford 3-(3-(4-(piperidin-4-ylmethyl)piperazine-1-carbonyl)phenyl)piperidine-2,6-dione dihydrochloride (116 mg) as a beige solid.

LC-MS (ESI): m/z=399.3 [M+H]⁺.

### Preparation of 2-((6-((5-chloro-2-(4-((4-(3-(2,6-dioxopiperidin-3-yl)benzoyl)piperazin-1-yl)methyl)piperidin-1-yl)pyrimidin-4-yl)amino)-1-methyl-2-oxo-1,2-dihydroquinolin-3-yl)oxy)-N-methylacetamide

A stirred mixture of 2-((6-((2,5-dichloropyrimidin-4-yl)amino)-1-methyl-2-oxo-1,2-dihydroquinolin-3-yl)oxy)-N-methylacetamide (40.0 mg, 98.0 μmol), 3-(3-(4-(piperidin-4-ylmethyl)piperazine-1-carbonyl)phenyl)piperidine-2,6-dione dihydrochloride (50.8 mg, 127 μmol), and CsF (44.7 mg, 294 μmol) in DMSO (980 μL) was warmed to 75° C. for 3 hours, then treated with DIEA (50.7 mg, 68.3 μL, 392 μmol) and stirred at 75° C. for 17 hours. The reaction mixture was cooled to room temperature, filtered, and purified by prep-HPLC to afford 2-((6-((5-chloro-2-(4-((4-(3-(2,6-dioxopiperidin-3-yl)benzoyl)piperazin-1-yl)methyl)piperidin-1-yl)pyrimidin-4-yl)amino)-1-methyl-2-oxo-1,2-dihydroquinolin-3-yl)oxy)-N-methylacetamide (23.7 mg) as a white solid.

LC-MS (ESI): m/z=770.5 [M+H]⁺.

¹H NMR (400 MHZ, DMSO-d₆) δ 10.85 (s, 1H), 8.81 (s, 1H), 8.03 (s, 1H), 7.94 (d, J=4.7 Hz, 1H), 7.92 (d, J=2.4 Hz, 1H), 7.75 (dd, J=9.1, 2.4 Hz, 1H), 7.47 (d, J=9.2 Hz, 1H), 7.40 (t, J=7.6 Hz, 1H), 7.34-7.26 (m, 2H), 7.24 (s, 1H), 7.10 (s, 1H), 4.57 (s, 2H), 4.48 (d, J=12.8 Hz, 2H), 3.93 (dd, J=12.0, 4.9 Hz, 1H), 3.75-3.65 (m, 3H), 3.60 (s, 3H), 2.83 (t, J=12.4 Hz, 2H), 2.67 (s, 3H), 2.66-2.62 (m, 2H), 2.44-2.36 (m, 4H), 2.35-2.27 (m, 2H), 2.19 (dd, J=23.7, 5.5 Hz, 2H), 2.11-2.00 (m, 1H), 1.74 (d, J=13.1 Hz, 3H), 1.02 (d, J=12.6 Hz, 2H).

### Example 86. Synthesis of 2-((6-((5-chloro-2-((3S,5R)-4-((1-(3-(2,6-dioxopiperidin-3-yl)-1-methyl-1H-indazol-7-yl)piperidin-4-yl)methyl)-3,5-dimethylpiperazin-1-yl)pyrimidin-4-yl)amino)-1-methyl-2-oxo-1,2-dihydroquinolin-3-yl)oxy)-N-methylacetamide (Compound 336a)

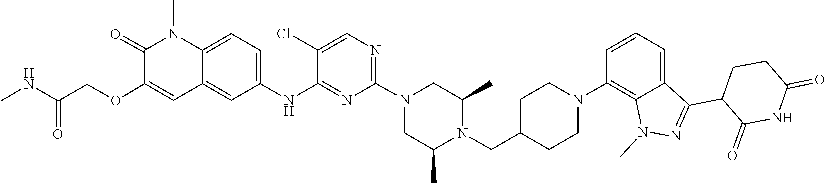 [SMILES: CNC(=O)COc1cc2cc(Nc3nc(N4C[C@@H](C)N(CC5CCN(c6cccc7c(C8CCC(=O)NC8=O)nn(C)c67)CC5)[C@@H](C)C4)ncc3Cl)ccc2n(C)c1=O]

### Preparation of tert-butyl (3S,5R)-4-((1-((benzyloxy)carbonyl)piperidin-4-yl)methyl)-3,5-dimethylpiperazine-1-carboxylate

To a stirred solution of tert-butyl (3S,5R)-3,5-dimethylpiperazine-1-carboxylate (6.0 g, 28.0 mmol) and benzyl 4-formylpiperidine-1-carboxylate (8.30 g, 33.6 mmol) in DCM (120 mL), acetic acid (1.60 mL, 28.0 mmol) was added, and the reaction was stirred for 3 hours. Sodium triacetoxyborohydride (11.9 g, 56.0 mmol) was added, and the resultant reaction mixture was stirred at room temperature for 16 hours. The reaction mixture was quenched with NH₄Cl (70 mL) and extracted with DCM (2×60 mL). The combined organic layers were washed with brine solution (50 mL), dried over anhydrous Na₂SO₄, filtered, concentrated under reduced pressure to afford the crude product. The crude product was purified by flash chromatography (230-400 silica gel; 25-35% EtOAc in petroleum ether) to afford tert-butyl (3S,5R)-4-((1-((benzyloxy)carbonyl)piperidin-4-yl)methyl)-3,5-dimethylpiperazine-1-carboxylate (2.30 g) as a yellow sticky compound.

LC-MS (ESI): m/z=446.35 [M+H]⁺.

### Preparation of tert-butyl (3S,5R)-3,5-dimethyl-4-(piperidin-4-ylmethyl)piperazine-1-carboxylate

A solution of tert-butyl (3S,5R)-4-((1-((benzyloxy)carbonyl)piperidin-4-yl)methyl)-3,5-dimethylpiperazine-1-carboxylate (2.30 g, 5.16 mmol) in THF (25 mL) was degassed with nitrogen for 5 minutes. 20% Pd(OH)₂ (1.15 g) was added, and the reaction was put under hydrogen atmosphere (80 psi). The reaction was stirred at room temperature for 16 hours. The reaction mixture was diluted with THF (50 mL) and filtered through a celite bed. The celite was washed with THF:DCM (1:1, 300 mL). The filtrate was concentrated and dried to afford tert-butyl (3S,5R)-3,5-dimethyl-4-(piperidin-4-ylmethyl)piperazine-1-carboxylate (1.44 g) as a brown gummy solid. ¹H NMR, 400 MHZ, DMSO-d₆-D₂O-Ex: δ 3.12 (d, J=11.60 Hz, 2H), 2.65-2.62 (m, 3H), 2.38-2.28 (m, 5H), 1.78 (d, J=12.40 Hz, 4H), 1.39 (s, 11H), 1.11 (q, J=10.80 Hz, 2H), 0.98 (d, J=6.00 Hz, 7H).

### Preparation of tert-butyl (3S,5R)-4-((1-(3-(2,6-bis(benzyloxy)pyridin-3-yl)-1-methyl-1H-indazol-7-yl)piperidin-4-yl)methyl)-3,5-dimethylpiperazine-1-carboxylate

To a stirred solution of 3-(2,6-bis(benzyloxy)pyridin-3-yl)-7-bromo-1-methyl-1H-indazole (0.70 g, 1.39 mmol) and tert-butyl (3S,5R)-3,5-dimethyl-4-(piperidin-4-ylmethyl)piperazine-1-carboxylate (1.30 g, 4.17 mmol) in dioxane (14 mL), cesium carbonate (1.37 g, 4.20 mmol) was added, and the reaction was degassed with argon for 15 minutes. Pd-PEPPSI-IHeptCl (0.06 g, 0.06 mmol) was added, and the reaction was stirred at 100° C. for 16 hours. The reaction mixture was filtered through a celite bed, and the celite was washed with ethyl acetate (80 mL). The filtrate was concentrated and dried under vacuum to afford crude product. The crude product was purified by flash chromatography (230-400 silica gel; 35-45% EtOAc in petroleum ether) to afford tert-butyl (3S,5R)-4-((1-(3-(2,6-bis(benzyloxy)pyridin-3-yl)-1-methyl-1H-indazol-7-yl)piperidin-4-yl)methyl)-3,5-dimethylpiperazine-1-carboxylate (0.40 g) as a yellow sticky compound.

LC-MS (ESI): m/z=731.50 [M+H]⁺.

### Preparation of tert-butyl (3S,5R)-4-((1-(3-(2,6-dioxopiperidin-3-yl)-1-methyl-1H-indazol-7-yl)piperidin-4-yl)methyl)-3,5-dimethylpiperazine-1-carboxylate

To a stirred solution of tert-butyl (3S,5R)-4-((1-(3-(2,6-bis(benzyloxy)pyridin-3-yl)-1-methyl-1H-indazol-7-yl)piperidin-4-yl)methyl)-3,5-dimethylpiperazine-1-carboxylate (0.40 g, 0.54 mmol) in THF (16 mL), 20% Pd(OH)₂ (0.40 g) was added. The resultant reaction mixture was put under hydrogen atmosphere (80 psi) and stirred at room temperature for 5 hours. The reaction mixture was filtered through a celite bed, and the celite was washed with 80 mL of THF:DCM (1:1). The filtrate was concentrated and dried to afford tert-butyl (3S,5R)-4-((1-(3-(2,6-dioxopiperidin-3-yl)-1-methyl-1H-indazol-7-yl)piperidin-4-yl)methyl)-3,5-dimethylpiperazine-1-carboxylate (0.36 g) as a brown gummy solid.

LC-MS (ESI): m/z=553.51 [M+H]⁺.

### Preparation of 3-(7-(4-(((2S,6R)-2,6-dimethylpiperazin-1-yl)methyl)piperidin-1-yl)-1-methyl-1H-indazol-3-yl)piperidine-2,6-dione

A stirred solution of tert-butyl (3S,5R)-4-((1-(3-(2,6-dioxopiperidin-3-yl)-1-methyl-1H-indazol-7-yl)piperidin-4-yl)methyl)-3,5-dimethylpiperazine-1-carboxylate (0.35 g, 0.68 mmol) in DCM (7 mL) was cooled to 0° C., and TFA (3.50 mL) was added. The reaction was allowed to warm to room temperature over 2 hours with stirring. The reaction mixture was concentrated under reduced pressure and co-distilled with DCM (3×5 mL) to obtain crude product. The crude product was triturated with n-pentane and dried to afford 3-(7-(4-(((2S,6R)-2,6-dimethylpiperazin-1-yl)methyl)piperidin-1-yl)-1-methyl-1H-indazol-3-yl)piperidine-2,6-dione (0.31 g) as a brown gummy solid.

LC-MS (ESI): m/z=453.59 [M+H]⁺.

### Preparation of 2-((6-((5-chloro-2-((3S,5R)-4-((1-(3-(2,6-dioxopiperidin-3-yl)-1-methyl-1H-indazol-7-yl)piperidin-4-yl)methyl)-3,5-dimethylpiperazin-1-yl)pyrimidin-4-yl)amino)-1-methyl-2-oxo-1,2-dihydroquinolin-3-yl)oxy)-N-methylacetamide

To a stirred solution of 3-(7-(4-(((2S,6R)-2,6-dimethylpiperazin-1-yl)methyl)piperidin-1-yl)-1-methyl-1H-indazol-3-yl)piperidine-2,6-dione (0.15 g, 0.33 mmol) in DMSO (1.5 mL), DIPEA (1.5 mL) and 2-((6-((2,5-dichloropyrimidin-4-yl)amino)-1-methyl-2-oxo-1,2-dihydroquinolin-3-yl)oxy)-N-methylacetamide (0.05 g, 0.12 mmol) were added. The reaction was stirred at 100° C. for 6 hours. The solvent was removed using a centrifugal evaporator to obtain crude product. The crude product was purified by prep-HPLC to afford 2-((6-((5-chloro-2-((3S,5R)-4-((1-(3-(2,6-dioxopiperidin-3-yl)-1-methyl-1H-indazol-7-yl)piperidin-4-yl)methyl)-3,5-dimethylpiperazin-1-yl)pyrimidin-4-yl)amino)-1-methyl-2-oxo-1,2-dihydroquinolin-3-yl)oxy)-N-methylacetamide (0.02 g) as a pale brown solid.

LC-MS (ESI): m/z=824.58 [M+H]⁺.

¹H NMR (400 MHZ, DMSO-d₆): δ 10.85 (s, 1H), 8.87 (s, 1H), 8.05 (s, 1H), 7.97-7.94 (m, 2H), 7.73 (dd, J=2.00, 9.00 Hz, 1H), 7.47 (d, J=9.20 Hz, 1H), 7.36 (t, J=4.40 Hz, 1H), 7.17 (s, 1H), 7.01-6.99 (m, 2H), 4.55 (s, 2H), 4.33 (q, J=5.20 Hz, 1H), 4.24-4.17 (m, 5H), 3.68 (s, 3H), 3.21-3.16 (m, 2H), 2.85-2.62 (m, 11H), 2.44-2.14 (m, 4H), 1.90 (d, J=11.20 Hz, 2H), 1.53-1.50 (m, 1H), 1.48-1.30 (m, 2H), 1.10 (d, J=56.00 Hz, 6H).

### Example 87. Synthesis of 2-((6-((5-chloro-2-(4-(2-(1-(3-(2,6-dioxopiperidin-3-yl)-1-methyl-1H-indazol-7-yl)piperidin-4-yl) propan-2-yl)piperazin-1-yl)pyrimidin-4-yl)amino)-1-methyl-2-oxo-1,2-dihydroquinolin-3-yl)oxy)-N-methylacetamide (Compound 337)

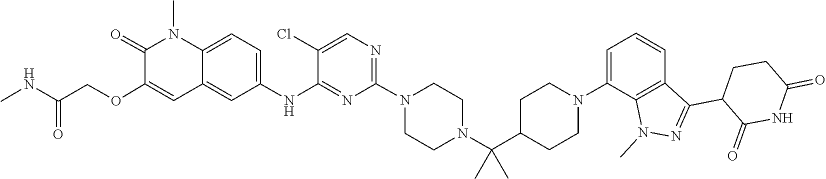 [SMILES: CNC(=O)COc1cc2cc(Nc3nc(N4CCN(C(C)(C)C5CCN(c6cccc7c(C8CCC(=O)NC8=O)nn(C)c67)CC5)CC4)ncc3Cl)ccc2n(C)c1=O]

### Preparation of benzyl 3,4-dihydroxypyrrolidine-1-carboxylate

To stirred solution of benzyl 2,5-dihydro-1H-pyrrole-1-carboxylate (17.6 g, 86.7 mmol) and N-methyl morpholine N-oxide (10.2 g, 86.7 mmol) in tetrahydrofuran (264 mL) was added dropwise a solution of osmium tetraoxide (4.40 g, 17.3 mmol) in t-butanol (106 mL) at 0° C. After stirring for 24 hours, the reaction mixture was quenched with cold water (100 mL) and extracted with ethyl acetate (3×150 mL). The organic layer was separated, dried over anhydrous sodium sulfate, filtered, and concentrated to get crude product. The crude product was purified by flash chromatography (10% methanol in dichloromethane) to afford benzyl 3,4-dihydroxypyrrolidine-1-carboxylate (7.75 g) as a brown gum.

LC-MS (ESI): m/z=238.06 [M+H]⁺.

### Preparation of benzyl bis(2-oxoethyl) carbamate

To a stirred solution of benzyl 3,4-dihydroxypyrrolidine-1-carboxylate (7.75 g, 32.7 mmol) in dichloromethane (78 mL) was added sodium periodate (10.5 g, 49.0 mmol) portion wise at 0° C. The resulting reaction mixture was stirred at room temperature for 1 hour. The reaction mixture was quenched with cold water (100 mL) and extracted with ethyl acetate (3×150 mL). The organic layer was separated, dried over anhydrous sodium sulfate, filtered, and concentrated to obtain benzyl bis(2-oxoethyl) carbamate (6.1 g) as a brown gum.

LC-MS (ESI): m/z=234.20 [M−H]⁻.

### Preparation of tert-butyl 4-(2-(1-((benzyloxy)carbonyl)piperidin-4-yl) propan-2-yl)piperazine-1-carboxylate

To a solution of benzyl bis(2-oxoethyl) carbamate (6.10 g, 26.0 mmol), tert-butyl 4-(2-aminopropan-2-yl)piperidine-1-carboxylate (3.14 g, 13.0 mmol) in methanol (122 mL) was added acetic acid (1.55 mL, 60.1 mmol) at 0° C. The reaction mixture was stirred at room temperature for 2 hours. 2-Methylpyridine borane complex (5.55 g, 51.9 mmol) was added portion wise, and the reaction was stirred at room temperature for 16 hours. The reaction mixture was quenched with ice-cold water (100 mL) and extracted with dichloromethane (3×150 mL). The combined organic layers were washed with brine solution (100 mL), dried over anhydrous Na₂SO₄, filtered, and concentrated under vacuum to get crude product. The crude product was purified by prep HPLC to afford tert-butyl 4-(2-(1-((benzyloxy)carbonyl)piperidin-4-yl) propan-2-yl)piperazine-1-carboxylate (0.55 g) as an off white solid.

LC-MS (ESI): m/z=446.61 [M+H]⁺.

### Preparation of tert-butyl 4-(2-(piperidin-4-yl) propan-2-yl)piperazine-1-carboxylate

To a stirred solution of tert-butyl 4-(2-(1-((benzyloxy)carbonyl)piperidin-4-yl) propan-2-yl)piperazine-1-carboxylate (0.55 g, 1.12 mmol) in 2-propanol (11 mL) was added 20% Pd(OH)₂ (0.55 g). The reaction was put under hydrogen atmosphere (20 psi) and stirred at room temperature for 16 hours. The reaction mixture was diluted with methanol (120 mL) and filtered through a celite bed. The filtrate was concentrated under vacuum to obtain tert-butyl 4-(2-(piperidin-4-yl) propan-2-yl)piperazine-1-carboxylate (0.28 g) as a brown gum.

LC-MS (ESI): m/z=312.21 [M+H]⁺.

### Preparation of tert-butyl 4-(2-(1-(3-(2,6-bis(benzyloxy)pyridin-3-yl)-1-methyl-1H-indazol-7-yl)piperidin-4-yl) propan-2-yl)piperazine-1-carboxylate

To a stirred solution of 3-(2,6-bis(benzyloxy)pyridin-3-yl)-7-bromo-1-methyl-1H-indazole (0.22 g, 0.44 mmol) and tert-butyl 4-(2-(piperidin-4-yl) propan-2-yl)piperazine-1-carboxylate (0.28 g, 0.89 mmol) in 1,4-dioxane (4.5 mL) was added cesium carbonate (0.43 g, 1.34 mmol). The reaction was degassed with argon for 10 minutes, Pd-PEPPSI-iHeptCl (0.02 g, 0.02 mmol) was added, and the reaction was heated at 100° C. for 16 hours. The reaction mixture was filtered through a celite bed and concentrated to obtain crude product. The crude product was purified by flash column chromatography (30% ethyl acetate in petroleum ether) to afford tert-butyl 4-(2-(1-(3-(2,6-bis(benzyloxy)pyridin-3-yl)-1-methyl-1H-indazol-7-yl)piperidin-4-yl) propan-2-yl)piperazine-1-carboxylate (0.18 g) as a brown gum.

LC-MS (ESI): m/z=731.90 [M+H]⁺.

### Preparation of tert-butyl 4-(2-(1-(3-(2,6-dioxopiperidin-3-yl)-1-methyl-1H-indazol-7-yl)piperidin-4-yl) propan-2-yl)piperazine-1-carboxylate

To a stirred solution of tert-butyl 4-(2-(1-(3-(2,6-bis(benzyloxy)pyridin-3-yl)-1-methyl-1H-indazol-7-yl)piperidin-4-yl) propan-2-yl)piperazine-1-carboxylate (0.18 g, 0.24 mmol) in tetrahydrofuran (3.6 mL) was added 20% Pd(OH)₂ (0.18 g). The reaction was put under hydrogen atmosphere (20 psi) and stirred at room temperature for 8 hours. The reaction mixture was diluted with dichloromethane (80 mL) and filtered through a celite bed. The filtrate was concentrated under vacuum to afford tert-butyl 4-(2-(1-(3-(2,6-dioxopiperidin-3-yl)-1-methyl-1H-indazol-7-yl)piperidin-4-yl) propan-2-yl)piperazine-1-carboxylate as (0.13 g) as a brown solid.

LC-MS (ESI): m/z=553.69 [M+H]⁺.

### Preparation of 3-(1-methyl-7-(4-(2-(piperazin-1-yl) propan-2-yl)piperidin-1-yl)-1H-indazol-3-yl)piperidine-2,6-dione

To a stirred solution of tert-butyl 4-(2-(1-(3-(2,6-dioxopiperidin-3-yl)-1-methyl-1H-indazol-7-yl)piperidin-4-yl) propan-2-yl)piperazine-1-carboxylate (0.13 g, 1.31 mmol) in dichloromethane (2.6 mL) was added TFA (0.4 mL). The reaction was stirred at room temperature for 1 hour. The reaction mixture was concentrated and co-distilled with petroleum ether to obtain 3-(1-methyl-7-(4-(2-(piperazin-1-yl) propan-2-yl)piperidin-1-yl)-1H-indazol-3-yl)piperidine-2,6-dione (0.15 g, TFA salt) as a brown solid.

LC-MS (ESI): m/z=453.58 [M+H]⁺.

### Preparation of 2-((6-((5-chloro-2-(4-(2-(1-(3-(2,6-dioxopiperidin-3-yl)-1-methyl-1H-indazol-7-yl)piperidin-4-yl) propan-2-yl)piperazin-1-yl)pyrimidin-4-yl)amino)-1-methyl-2-oxo-1,2-dihydroquinolin-3-yl)oxy)-N-methylacetamide

To a stirred solution 3-(1-methyl-7-(4-(2-(piperazin-1-yl) propan-2-yl)piperidin-1-yl)-1H-indazol-3-yl)piperidine-2,6-dione (0.075 g, 0.13 mmol) and 2-((6-((2,5-dichloropyrimidin-4-yl)amino)-1-methyl-2-oxo-1,2-dihydroquinolin-3-yl)oxy)-N-methylacetamide (0.033 g, 0.08 mmol) in DMSO (1.5 mL), DIPEA (0.23 mL, 1.36 mmol) was added. The reaction was stirred at 100° C. for 3 hours. Solvent was removed using a centrifugal evaporator to obtain crude product. The crude product was purified by prep HPLC to afford 2-((6-((5-chloro-2-(4-(2-(1-(3-(2,6-dioxopiperidin-3-yl)-1-methyl-1H-indazol-7-yl)piperidin-4-yl) propan-2-yl)piperazin-1-yl)pyrimidin-4-yl)amino)-1-methyl-2-oxo-1,2-dihydroquinolin-3-yl)oxy)-N-methylacetamide (0.020 g) as an off-white solid.

LC-MS (ESI): m/z=824.58 [M+H]⁺.

¹H NMR (400 MHZ, DMSO-d₆): δ 10.80 (s, 1H), 8.81 (s, 1H), 8.03 (s, 1H), 7.95 (s, 1H), 7.88 (d, J=4.40 Hz, 1H), 7.80 (s, 1H), 7.80-7.78 (d, J=8.80 Hz, 1H), 7.49 (d, J=9.20 Hz, 1H), 7.39-7.37 (m, 1H), 7.14 (s, 1H), 7.05-7.03 (s, 2H), 4.58-4.61 (m, 3H), 4.25-4.32 (m, 3H), 3.86 (s, 3H), 2.50-2.58 (m, 15H), 2.33 (s, 2H), 1.99 (s, 3H), 1.36 (s, 1H), 1.11-1.14 (m, 2H), 0.85 (s, 6H).

### Example 88. Synthesis of 2-((6-((5-chloro-2-(4-(4-(3-(2,6-dioxopiperidin-3-yl)-1-methyl-1H-indazol-7-yl)piperazine-1-carbonyl)piperidin-1-yl)pyrimidin-4-yl)amino)-1-methyl-2-oxo-1,2-dihydroquinolin-3-yl)oxy)-N-methylacetamide (Compound 327)

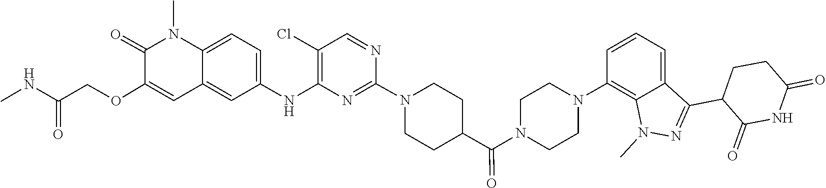 [SMILES: CNC(=O)COc1cc2cc(Nc3nc(N4CCC(C(=O)N5CCN(c6cccc7c(C8CCC(=O)NC8=O)nn(C)c67)CC5)CC4)ncc3Cl)ccc2n(C)c1=O]

### Preparation of tert-butyl 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylate

A mixture of 2-((6-((2,5-dichloropyrimidin-4-yl)amino)-1-methyl-2-oxo-1,2-dihydroquinolin-3-yl)oxy)-N-methylacetamide (163 mg, 400 μmol), tert-butyl piperidine-4-carboxylate hydrochloride (133 mg, 600 μmol), and DIPEA (210 μL, 1.20 mmol) in DMF (4 mL) was stirred at 95° C. for 2.8 hours. The reaction mixture was diluted with water and extracted three times with EtOAc. The combined organics were washed with brine, dried over sodium sulfate, filtered, and concentrated under reduced pressure. The resulting solid was triturated in EtOH (10 mL) and collected by filtration, washing with EtOH (5 mL). The solid was dried under vacuum to give crude tert-butyl 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylate (144 mg) as a tan solid.

LC-MS (ESI): m/z=557.4 [M+H]⁺.

### Preparation of 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylic acid

To a suspension of tert-butyl 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylate (144 mg, 259 μmol) in 1,4-dioxane (1 mL) was added hydrogen chloride (4 M in dioxane, 1.29 mL, 5.17 mmol). The mixture was stirred at 40° C. overnight. The reaction mixture was removed from heat and was concentrated under reduced pressure to give crude 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylic acid (169 mg).

LC-MS (ESI): m/z=501.3 [M+H]⁺.

### Preparation of tert-butyl 4-(3-(2,6-bis(benzyloxy)pyridin-3-yl)-1-methyl-1H-indazol-7-yl)piperazine-1-carboxylate

To a solution of 7-bromo-3-(2,6-dibenzyloxy-3-pyridyl)-1-methyl-indazole (10 g, 20.0 mmol) in dioxane (100 mL), Pd₂(dba)₃ (1.83 g, 2.00 mmol), RuPhos (1.87 g, 4.00 mmol), Cs₂CO₃ (19.5 g, 60.0 mmol), and tert-butyl piperazine-1-carboxylate (11.2 g, 60.0 mmol) were added. The suspension was degassed and purged with nitrogen 3 times. The mixture was stirred under nitrogen at 100° C. for 3 hours. The reaction mixture was concentrated under reduced pressure to remove solvent. The residue was diluted with H₂O (100 mL) and extracted with ethyl acetate (100 mL×3). The combined organic layers were washed with H₂O 300 mL (100 mL×3), dried over Na₂SO₄, filtered, and concentrated under reduced pressure to obtain crude product. The crude product was purified by column chromatography (SiO₂, petroleum ether/ethyl acetate=100/1 to 4/1) to afford tert-butyl 4-(3-(2,6-bis(benzyloxy)pyridin-3-yl)-1-methyl-1H-indazol-7-yl)piperazine-1-carboxylate (8 g) as a yellow oil.

LC-MS (ESI): m/z=606.8 [M+H]⁺.

### Preparation of tert-butyl 4-(3-(2,6-dioxopiperidin-3-yl)-1-methyl-1H-indazol-7-yl)piperazine-1-carboxylate

To a solution of tert-butyl 4-(3-(2,6-bis(benzyloxy)pyridin-3-yl)-1-methyl-1H-indazol-7-yl)piperazine-1-carboxylate (8 g, 13.2 mmol) in EtOH (50 mL) and THF (50 mL), AcOH (793 mg, 13.2 mmol, 755 μL), Pd(OH)₂ (7 g, 9.97 mmol, 20% w/v), and Pd/C (7 g, 6.60 mmol, w/v) were added under a nitrogen atmosphere. The suspension was degassed and purged with hydrogen 3 times. The mixture was stirred under a hydrogen atmosphere (50 psi) at 50° C. for 12 hours. The reaction mixture was filtered and concentrated under reduced pressure to obtain tert-butyl 4-(3-(2,6-dioxopiperidin-3-yl)-1-methyl-1H-indazol-7-yl)piperazine-1-carboxylate (3 g) as a white solid.

LC-MS (ESI): m/z=328.1 [M+H-100]⁺.

### Preparation of 3-(1-methyl-7-(piperazin-1-yl)-1H-indazol-3-yl)piperidine-2,6-dione (HCl salt

To a stirred solution of tert-butyl 4-(3-(2,6-dioxopiperidin-3-yl)-1-methyl-1H-indazol-7-yl)piperazine-1-carboxylate (3.1 g, 7.25 mmol) in DCM, 4 N HCl in dioxane (4 M, 1.81 mL) was added. The mixture was stirred at 25° C. for 0.5 hours. The reaction mixture was concentrated under reduced pressure to remove solvent. The crude product was triturated with ethyl acetate to afford 3-(1-methyl-7-(piperazin-1-yl)-1H-indazol-3-yl)piperidine-2,6-dione (HCl salt)(1531 mg) as a white solid.

LC-MS (ESI): m/z=328.1 [M+H]⁺.

### Preparation of 2-((6-((5-chloro-2-(4-(4-(3-(2,6-dioxopiperidin-3-yl)-1-methyl-1H-indazol-7-yl)piperazine-1-carbonyl)piperidin-1-yl)pyrimidin-4-yl)amino)-1-methyl-2-oxo-1,2-dihydroquinolin-3-yl)oxy)-N-methylacetamide

A mixture of 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylic acid (84.5 mg, 127 μmol), 3-(1-methyl-7-(piperazin-1-yl)-1H-indazol-3-yl)piperidine-2,6-dione hydrochloride (50.6 mg, 139 μmol), DIPEA (66.1 μL, 380 μmol), and HATU (57.7 mg, 152 μmol) in DMF (1 mL) was stirred at 40° C. overnight. The reaction mixture was filtered through a 0.45 μm PTFE syringe filter. The filtrate was purified by preparative HPLC to afford 2-((6-((5-chloro-2-(4-(4-(3-(2,6-dioxopiperidin-3-yl)-1-methyl-1H-indazol-7-yl)piperazine-1-carbonyl)piperidin-1-yl)pyrimidin-4-yl)amino)-1-methyl-2-oxo-1,2-dihydroquinolin-3-yl)oxy)-N-methylacetamide, TFA (36.9 mg) as a white solid.

LC-MS (ESI): m/z=810.5 [M+H]⁺.

¹H NMR (400 MHZ, DMSO-d₆) δ 10.88 (s, 1H), 9.09 (s, 1H), 8.10 (s, 1H), 7.92 (dd, J=10.9, 3.6 Hz, 2H), 7.75 (dd, J=9.1, 2.4 Hz, 1H), 7.49 (d, J=9.1 Hz, 1H), 7.43 (dd, J=7.4, 1.5 Hz, 1H), 7.13 (s, 1H), 7.10-6.95 (m, 2H), 4.57 (s, 2H), 4.45 (d, J=12.8 Hz, 2H), 4.35 (dd, J=9.7, 5.1 Hz, 1H), 4.27 (s, 3H), 3.85 (br s, 8H), 3.68 (s, 3H), 3.21 (s, 1H), 3.00 (t, J=12.2 Hz, 2H), 2.74-2.56 (m, 5H), 2.40-2.26 (m, 1H), 2.23-2.10 (m, 1H), 1.73 (d, J=12.8 Hz, 2H), 1.54 (s, 2H).

### Example 89. Synthesis of 2-((6-((5-chloro-2-(4-(4-(3-(2,6-dioxopiperidin-3-yl)-1-methyl-1H-indazol-6-yl)piperazine-1-carbonyl)piperidin-1-yl)pyrimidin-4-yl)amino)-1-methyl-2-oxo-1,2-dihydroquinolin-3-yl)oxy)-N-methylacetamide (Compound 328)

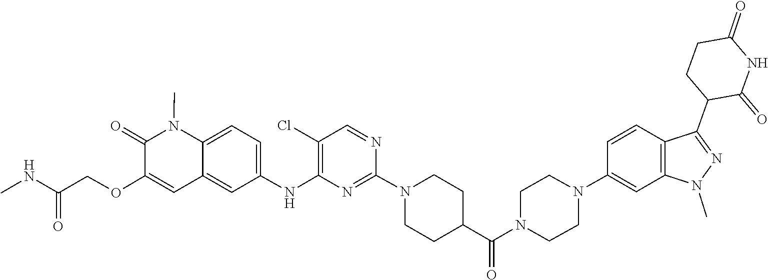 [SMILES: CNC(=O)COc1cc2cc(Nc3nc(N4CCC(C(=O)N5CCN(c6ccc7c(C8CCC(=O)NC8=O)nn(C)c7c6)CC5)CC4)ncc3Cl)ccc2n(C)c1=O]

### Preparation of tert-butyl 4-[3-(2,6-dibenzyloxy-3-pyridyl)-1-methyl-indazol-6-yl]piperazine-1-carboxylate

A mixture of 6-bromo-3-(2,6-dibenzyloxy-3-pyridyl)-1-methyl-indazole (5.0 g, 9.99 mmol), tert-butyl piperazine-1-carboxylate (1.86 g, 9.99 mmol), RuPhos (933 mg, 2.00 mmol), Cs₂CO₃ (9.77 g, 30.0 mmol) and Pd (dba)₂ (575 mg, 999 μmol) in dioxane (60 mL) was degassed and purged with nitrogen 3 times. The reaction was stirred at 100° C. for 2 hours under nitrogen atmosphere. The reaction mixture was concentrated under reduced pressure to obtain crude product. The crude product was purified by flash column chromatography (silica gel, 0˜50% ethyl acetate/petroleum ether) to afford tert-butyl 4-[3-(2,6-dibenzyloxy-3-pyridyl)-1-methyl-indazol-6-yl]piperazine-1-carboxylate (5.0 g) as a yellow solid.

LC-MS (ESI): m/z=606.5 [M+H]⁺.

### Preparation of tert-butyl 4-[3-(2,6-dioxo-3-piperidyl)-1-methyl-indazol-6-yl]piperazine-1-carboxylate

To a solution of tert-butyl 4-[3-(2,6-dibenzyloxy-3-pyridyl)-1-methyl-indazol-6-yl]piperazine-1-carboxylate (9 g, 14.9 mmol) in THF (10 mL) and EtOH (10 mL), Pd/C (0.5 g, 1.49 mmol, 10% w/v), Pd(OH)₂ (563 mg, 4.01 mmol), and AcOH (2.68 g, 44.6 mmol, 2.55 mL) were added under a nitrogen atmosphere. The suspension was degassed and purged with hydrogen 3 times. The mixture was stirred under hydrogen (50 psi) at 50° C. for 12 hours. The reaction mixture was filtered and concentrated under reduced pressure to obtain crude product. The crude product was purified by column chromatography (SiO₂, DCM:MeOH=1/0 to 10/1) to afford tert-butyl 4-[3-(2,6-dioxo-3-piperidyl)-1-methyl-indazol-6-yl]piperazine-1-carboxylate (4.4 g, 10.3 mmol) as a white solid.

¹H NMR (400 MHZ, DMSO-d₆): δ=10.86 (s, 1H), 7.53 (d, J=8.8 Hz, 1H), 6.98-6.83 (m, 2H), 4.27 (dd, J=4.8, 9.2 Hz, 1H), 3.90 (s, 3H), 3.50 (s, 4H), 3.19 (s, 4H), 2.65-2.59 (m, 2H), 2.33-2.13 (m, 2H), 1.44 (s, 9H).

### Preparation of 3-(1-methyl-6-piperazin-1-yl-indazol-3-yl)piperidine-2,6-dione (HCl salt

Tert-butyl 4-[3-(2,6-dioxo-3-piperidyl)-1-methyl-indazol-6-yl]piperazine-1-carboxylate (4.0 g, 9.36 mmol) was dissolved in a 4 N solution of HCl/dioxane (50 mL). The mixture was stirred at 25° C. for 12 hours. The reaction mixture was concentrated under reduced pressure to remove solvent to afford 3-(1-methyl-6-piperazin-1-yl-indazol-3-yl)piperidine-2,6-dione (HCl salt) (3.0 g) as a white solid.

LC-MS (ESI): m/z=328.1 [M+H]⁺.

### Preparation of 2-((6-((5-chloro-2-(4-(4-(3-(2,6-dioxopiperidin-3-yl)-1-methyl-1H-indazol-6-yl)piperazine-1-carbonyl)piperidin-1-yl)pyrimidin-4-yl)amino)-1-methyl-2-oxo-1,2-dihydroquinolin-3-yl)oxy)-N-methylacetamide

A mixture of 1-(5-chloro-4-((1-methyl-3-(2-(methylamino)-2-oxoethoxy)-2-oxo-1,2-dihydroquinolin-6-yl)amino)pyrimidin-2-yl)piperidine-4-carboxylic acid (84.5 mg, 127 μmol) (see, e.g., Example 88), 3-(1-methyl-6-piperazin-1-yl-indazol-3-yl)piperidine-2,6-dione hydrochloride (50.6 mg, 139 μmol), DIPEA (49.1 mg, 66.1 μL, 3 eq., 380 μmol), and HATU (57.7 mg, 1.2 eq., 152 μmol) in DMF (1 mL) was stirred at 40° C. overnight. The reaction mixture was filtered through a 0.45 μm PTFE syringe filter. The filtrate was purified by preparative HPLC to afford 2-((6-((5-chloro-2-(4-(4-(3-(2,6-dioxopiperidin-3-yl)-1-methyl-1H-indazol-6-yl)piperazine-1-carbonyl)piperidin-1-yl)pyrimidin-4-yl)amino)-1-methyl-2-oxo-1,2-dihydroquinolin-3-yl)oxy)-N-methylacetamide, TFA (16.5 mg) as a white solid.

LC-MS (ESI): m/z=810.5 [M+H]⁺.

¹H NMR (400 MHZ, DMSO-d₆) δ 10.85 (s, 1H), 9.09 (s, 1H), 8.10 (s, 1H), 7.91 (dd, J=10.8, 3.7 Hz, 2H), 7.75 (dd, J=9.1, 2.5 Hz, 1H), 7.51 (dd, J=18.1, 9.0 Hz, 2H), 7.13 (s, 1H), 6.95 (dd, J=9.0, 1.9 Hz, 1H), 6.90 (d, J=1.9 Hz, 1H), 4.57 (s, 2H), 4.52-4.40 (m, 2H), 4.27 (dd, J=9.3, 5.1 Hz, 1H), 3.90 (s, 3H), 3.74 (s, 2H), 3.67 (m, 5H), 3.22 (d, J=29.6 Hz, 4H), 3.00 (t, J=12.2 Hz, 3H), 2.63 (m, 5H), 2.38-2.23 (m, 1H), 2.16 (dd, J=13.3, 5.8 Hz, 1H), 1.72 (d, J=12.8 Hz, 2H), 1.53 (q, J=11.3 Hz, 2H).

### Example 90. Synthesis of 2-((6-((5-chloro-2-(5-((3-(2,6-dioxopiperidin-3-yl)-1-methyl-1H-indazol-6-yl)amino) octahydro-2H-isoindol-2-yl)pyrimidin-4-yl)amino)-1-methyl-2-oxo-1,2-dihydroquinolin-3-yl)oxy)-N-methylacetamide (Compound 338)

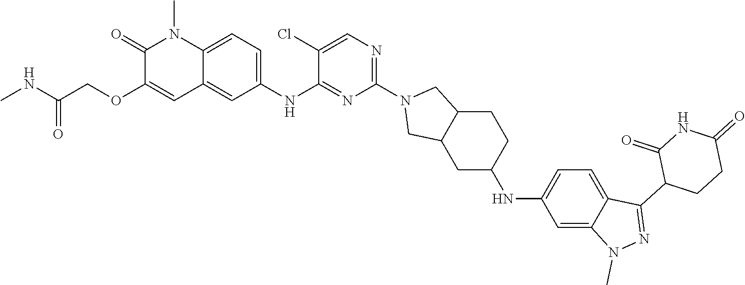 [SMILES: CNC(=O)COc1cc2cc(Nc3nc(N4CC5CCC(Nc6ccc7c(C8CCC(=O)NC8=O)nn(C)c7c6)CC5C4)ncc3Cl)ccc2n(C)c1=O]

### Preparation of tert-butyl 5-((3-(2,6-bis(benzyloxy)pyridin-3-yl)-1-methyl-1H-indazol-6-yl)amino) octahydro-2H-isoindole-2-carboxylate

A stirred solution of 3-(2,6-bis(benzyloxy)pyridin-3-yl)-6-bromo-1-methyl-1H-indazole (1.0 g, 2.0 mmol), tert-butyl 5-aminooctahydro-2H-isoindole-2-carboxylate (0.72 g, 3.0 mmol), and cesium carbonate (1.95 g, 6.0 mmol) in 1,4-dioxane (20 mL) was degassed with argon for 10 minutes. RuPhos (0.09 g, 0.20 mmol) and RuPhos-PdG3 (0.08 g, 0.10 mmol) were added, and the reaction was degassed for 5 minutes. The reaction was heated to 80° C. for 3 hours. The reaction mixture was diluted with ethyl acetate (200 mL) and filtered through a pad of celite. The filtrate was concentrated under vacuum to obtain crude product. The crude product was purified by flash column chromatography (SiO₂, 230-400, 30% ethyl acetate in petroleum ether) to obtain tert-butyl 5-((3-(2,6-bis(benzyloxy)pyridin-3-yl)-1-methyl-1H-indazol-6-yl)amino) octahydro-2H-isoindole-2-carboxylate (0.70 g) as an off-white solid.

LC-MS (ESI): m/z=660.71 [M+H]⁺.

### Preparation of tert-butyl 5-((3-(2,6-dioxopiperidin-3-yl)-1-methyl-1H-indazol-6-yl)amino) octahydro-2H-isoindole-2-carboxylate

To a stirred solution of tert-butyl 5-((3-(2,6-bis(benzyloxy)pyridin-3-yl)-1-methyl-1H-indazol-6-yl)amino) octahydro-2H-isoindole-2-carboxylate (0.70 g, 1.06 mmol) in THF (28.0 mL), 20% Pd(OH)₂/C (0.70 g) was added. The reaction was put under hydrogen atmosphere (60 psi) and stirred at room temperature for 8 hours. The reaction mixture was filtered through a pad of celite. The celite was washed with ethyl acetate (300 mL). The filtrate was concentrated under vacuum to obtain crude product. The crude product was washed with n-pentane (20 mL) and dried under vacuum to afford tert-butyl 5-((3-(2,6-dioxopiperidin-3-yl)-1-methyl-1H-indazol-6-yl)amino) octahydro-2H-isoindole-2-carboxylate (0.60 g) as a brown solid.

LC-MS (ESI): m/z=482.31 [M+H]⁺.

### Preparation of 3-(1-methyl-6-((octahydro-1H-isoindol-5-yl)amino)-1H-indazol-3-yl) piperidine-2,6-dione

To a stirred solution of tert-butyl 5-((3-(2,6-dioxopiperidin-3-yl)-1-methyl-1H-indazol-6-yl)amino) octahydro-2H-isoindole-2-carboxylate (0.60 g, 1.25 mmol) in DCM (12.0 mL), TFA (6.0 mL) was added at 0° C. The reaction was allowed to warm to room temperature over 3 hours with stirring. The reaction mixture was concentrated under vacuum to obtain crude product. The crude product was triturated with diethyl ether (30 mL) to afford 3-(1-methyl-6-((octahydro-1H-isoindol-5-yl)amino)-1H-indazol-3-yl)piperidine-2,6-dione (0.44 g) as a brown solid.

LC-MS (ESI): m/z=382.48 [M+H]⁺.

### Preparation of 2-((6-((5-chloro-2-(5-((3-(2,6-dioxopiperidin-3-yl)-1-methyl-1H-indazol-6-yl)amino) octahydro-2H-isoindol-2-yl)pyrimidin-4-yl)amino)-1-methyl-2-oxo-1,2-dihydroquinolin-3-yl)oxy)-N-methylacetamide

To a stirred solution of 3-(1-methyl-6-((octahydro-1H-isoindol-5-yl)amino)-1H-indazol-3-yl) piperidine-2,6-dione (0.22 g, 0.58 mmol) and 2-((6-((2,5-dichloropyrimidin-4-yl)amino)-1-methyl-2-oxo-1,2-dihydroquinolin-3-yl)oxy)-N-methylacetamide (0.09 g, 0.23 mmol) in DMSO (4 mL), N,N-diisopropylethylamine (0.8 mL, 4.61 mmol) was added. The reaction was heated at 100° C. for 4 hours. The solvent was removed using a centrifugal evaporator to obtain crude product. The crude product was purified by prep-HPLC to afford 2-((6-((5-chloro-2-(5-((3-(2,6-dioxopiperidin-3-yl)-1-methyl-1H-indazol-6-yl)amino) octahydro-2H-isoindol-2-yl)pyrimidin-4-yl)amino)-1-methyl-2-oxo-1,2-dihydroquinolin-3-yl)oxy)-N-methylacetamide (50 mg) as off-white solid.

LC-MS (ESI): m/z=753.50 [M+H]⁺.

¹H NMR data: (400 MHz, DMSO-d₆): δ 10.81 (s, 1H), 8.74 (s, 1H), 8.04 (d, J=5.20 Hz, 2H), 7.91-7.89 (m, 2H), 7.46-7.44 (m, 1H), 7.30 (d, J=8.80 Hz, 1H), 7.12 (d, J=8.80 Hz, 1H), 6.45 (br s, 1H), 6.39 (s, 1H), 5.60-5.62 (m, 1H), 4.56 (s, 2H), 4.16-4.15 (m, 1H), 3.81 (s, 3H), 3.68 (d, J=11.60 Hz, 3H), 3.51-3.46 (m, 5H), 2.59-2.58 (m, 5H), 2.50-2.48 (m, 2H), 2.16-2.14 (m, 1H), 2.11-2.07 (m, 1H), 1.87-1.75 (m, 4H), 1.34-1.30 (m, 2H).

### Example 91. Synthesis of 2-((6-((5-chloro-2-(4-(2-(1-(3-(2,6-dioxopiperidin-3-yl)-1-methyl-1H-indazol-6-yl)piperidin-4-yl) propan-2-yl)piperazin-1-yl)pyrimidin-4-yl)amino)-1-methyl-2-oxo-1,2-dihydroquinolin-3-yl)oxy)-N-methylacetamide (Compound 340)

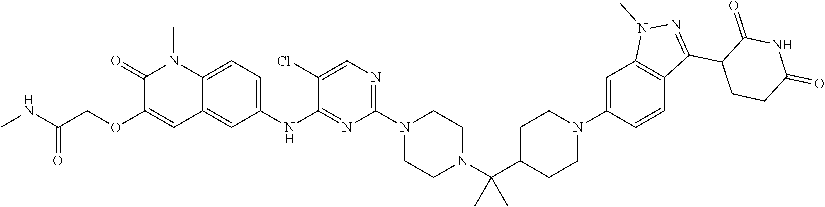 [SMILES: CNC(=O)COc1cc2cc(Nc3nc(N4CCN(C(C)(C)C5CCN(c6ccc7c(C8CCC(=O)NC8=O)nn(C)c7c6)CC5)CC4)ncc3Cl)ccc2n(C)c1=O]

### Preparation of tert-butyl 4-(2-(1-(3-(2,6-bis(benzyloxy)pyridin-3-yl)-1-methyl-1H-indazol-6-yl)piperidin-4-yl) propan-2-yl)piperazine-1-carboxylate

A solution of tert-butyl 4-(2-(piperidin-4-yl) propan-2-yl)piperazine-1-carboxylate (0.30 g, 0.59 mmol), 3-(2,6-bis(benzyloxy)pyridin-3-yl)-6-bromo-1-methyl-1H-indazole (0.37 g, 1.19 mmol) and cesium carbonate (0.58 g, 1.79 mmol) in 1,4-dioxane (20 mL) was degassed with argon for 5 minutes. Ruphos-Pd-G3 (0.02 g, 0.02 mmol) and Ruphos (0.03 g, 0.5 mmol) were added, and the reaction was degassed for an additional 5 minutes. The reaction mixture was heated at 100° C. for 16 hours. The reaction mixture was quenched with ice-cold water (50 mL) and extracted into ethyl acetate (3×100 mL). The combined organic layers were washed with brine solution, dried over anhydrous Na₂SO₄, filtered, and concentrated under vacuum to obtain crude product. The crude product was purified by column chromatography (SiO₂; 40% EtOAc in petroleum ether) to afford tert-butyl 4-(2-(1-(3-(2,6-bis(benzyloxy)pyridin-3-yl)-1-methyl-1H-indazol-6-yl)piperidin-4-yl) propan-2-yl)piperazine-1-carboxylate (0.4 g) as a pale brown semi-solid.

LC-MS (ESI): m/z=731.86 [M+H]⁺.

### Preparation of tert-butyl 4-(2-(1-(3-(2,6-dioxopiperidin-3-yl)-1-methyl-1H-indazol-6-yl)piperidin-4-yl) propan-2-yl)piperazine-1-carboxylate

To a stirred solution of tert-butyl 4-(2-(1-(3-(2,6-bis(benzyloxy)pyridin-3-yl)-1-methyl-1H-indazol-6-yl)piperidin-4-yl) propan-2-yl)piperazine-1-carboxylate (0.40 g, 0.54 mmol) in tetrahydrofuran (8 mL), 20% Pd(OH)₂ (0.40 g, 100% w/w) was added. The reaction was put under hydrogen atmosphere (20 psi) and stirred at room temperature for 8 hours. The reaction mixture was diluted with DCM (80 mL) and filtered through a celite bed. The filtrate was concentrated under vacuum to obtain tert-butyl 4-(2-(1-(3-(2,6-dioxopiperidin-3-yl)-1-methyl-1H-indazol-6-yl)piperidin-4-yl) propan-2-yl)piperazine-1-carboxylate (0.25 g) as a brown solid.

LC-MS (ESI): m/z=553.69 [M+H]⁺.

### Preparation of 3-(1-methyl-6-(4-(2-(piperazin-1-yl) propan-2-yl)piperidin-1-yl)-1H-indazol-3-yl)piperidine-2,6-dione (TFA salt

To a stirred solution of tert-butyl 4-(2-(1-(3-(2,6-dioxopiperidin-3-yl)-1-methyl-1H-indazol-6-yl)piperidin-4-yl) propan-2-yl)piperazine-1-carboxylate (0.25 g, 0.50 mmol) in DCM (5 mL), TFA (0.7 mL) was added. The reaction was stirred at room temperature for 1 hour. The reaction mixture was concentrated and co-distilled with petroleum ether to obtain 3-(1-methyl-6-(4-(2-(piperazin-1-yl) propan-2-yl)piperidin-1-yl)-1H-indazol-3-yl)piperidine-2,6-dione (0.24 g, TFA salt).

LC-MS (ESI): m/z=453.32 [M+H]⁺.

### Preparation 2-((6-((5-chloro-2-(4-(2-(1-(3-(2,6-dioxopiperidin-3-yl)-1-methyl-1H-indazol-6-yl)piperidin-4-yl) propan-2-yl)piperazin-1-yl)pyrimidin-4-yl)amino)-1-methyl-2-oxo-1,2-dihydroquinolin-3-yl)oxy)-N-methylacetamide

To a stirred solution 3-(1-methyl-6-(4-(2-(piperazin-1-yl) propan-2-yl)piperidin-1-yl)-1H-indazol-3-yl)piperidine-2,6-dione (TFA salt)(0.12 g, 0.21 mmol) and 2-((6-((2,5-dichloropyrimidin-4-yl)amino)-1-methyl-2-oxo-1,2-dihydroquinolin-3-yl)oxy)-N-methylacetamide (0.05 g, 0.13 mmol) in DMSO (2.4 mL), DIPEA (0.30 mL, 1.36 mmol) was added. The reaction was stirred at 100° C. for 3 hours. The solvent was removed using a centrifugal evaporator to obtain crude product. The crude product was purified by prep HPLC to afford 2-((6-((5-chloro-2-(4-(2-(1-(3-(2,6-dioxopiperidin-3-yl)-1-methyl-1H-indazol-6-yl)piperidin-4-yl) propan-2-yl)piperazin-1-yl)pyrimidin-4-yl)amino)-1-methyl-2-oxo-1,2-dihydroquinolin-3-yl)oxy)-N-methylacetamide (0.06 g) as an off-white solid.

LC-MS (ESI): m/z=824.58 [M+H]⁺.

¹H NMR (400 MHZ, DMSO-d₆): 10.90 (s, 1H), 8.80 (s, 1H), 8.33 (s, 1H), 8.03 (s, 1H), 7.94 (d, J=4.40 Hz, 1H), 7.88 (d, J=2.00 Hz, 1H), 7.80-7.78 (m, 1H), 7.50-7.48 (m, 2H), 7.13 (s, 1H), 6.91 (d, J=9.20 Hz, 1H), 6.82 (s, 1H), 4.60-4.57 (m, 3H), 4.25-4.24 (m, 1H), 3.89 (s, 3H), 3.68 (s, 3H), 3.27 (br s, 4H), 2.78-2.75 (m, 2H), 2.64-2.61 (m, 9H), 2.29-2.28 (m, 1H), 2.15-2.14 (m, 1H), 1.81-1.77 (m, 3H), 1.10-1.15 (m, 2H), 0.94 (s, 6H).

### Example 92. Synthesis of 2-((6-((5-chloro-2-(4-((1-(3-(2,6-dioxopiperidin-3-yl)-1-methyl-1H-indazol-7-yl)piperidin-4-yl)methyl)piperazin-1-yl) pyridin-4-yl)amino)-1-methyl-2-oxo-1,2-dihydroquinolin-3-yl)oxy)-N-methylacetamide (Compound 346)

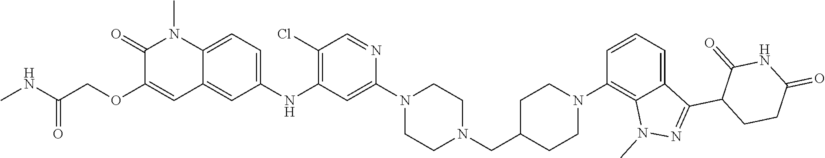 [SMILES: CNC(=O)COc1cc2cc(Nc3cc(N4CCN(CC5CCN(c6cccc7c(C8CCC(=O)NC8=O)nn(C)c67)CC5)CC4)ncc3Cl)ccc2n(C)c1=O]

### Preparation of 3-(7-(4-((4-(5-chloro-4-iodopyridin-2-yl)piperazin-1-yl)methyl)piperidin-1-yl)-1-methyl-1H-indazol-3-yl)piperidine-2,6-dione

A solution of 5-chloro-2-fluoro-4-iodopyridine (145 mg, 563 μmol), 3-(1-methyl-7-(4-(piperazin-1-ylmethyl)piperidin-1-yl)-1H-indazol-3-yl)piperidine-2,6-dione (237 mg)(see, e.g., Example 1), and DIEA (348 μL, 2.00 mmol) in DMA (1.50 mL) was warmed to 110° C. for 5 hours, then allowed to cool to room temperature. The reaction mixture was filtered and purified directly by reverse phase chromatography (10-100% MeCN—H₂O [+0.1% FA]) to afford 13-14, 3-(7-(4-((4-(5-chloro-4-iodopyridin-2-yl)piperazin-1-yl)methyl)piperidin-1-yl)-1-methyl-1H-indazol-3-yl)piperidine-2,6-dione (156 mg, 236 μmol).

LC-MS (ESI): m/z=662.3 [M+H]⁺.

### Preparation of 2-((6-((5-chloro-2-(4-((1-(3-(2,6-dioxopiperidin-3-yl)-1-methyl-1H-indazol-7-yl)piperidin-4-yl)methyl)piperazin-1-yl) pyridin-4-yl)amino)-1-methyl-2-oxo-1,2-dihydroquinolin-3-yl)oxy)-N-methylacetamide

A mixture of 2-((6-amino-1-methyl-2-oxo-1,2-dihydroquinolin-3-yl)oxy)-N-methylacetamide (45.2 mg, 173 μmol), 3-(7-(4-((4-(5-chloro-4-iodopyridin-2-yl)piperazin-1-yl)methyl)piperidin-1-yl)-1-methyl-1H-indazol-3-yl)piperidine-2,6-dione (99.2 mg, 150 μmol), BrettPhos-Pd-G3 (30.6 mg, 33.8 μmol), and Cs₂CO₃ (242 mg, 742 μmol) in 1,4-dioxane (1.50 mL) was sparged with N₂ (g) for 8 minutes, then warmed to 80° C. for 4 hours. The reaction mixture was cooled to room temperature, filtered, and purified by prep-HPLC to afford to afford 2-((6-((5-chloro-2-(4-((1-(3-(2,6-dioxopiperidin-3-yl)-1-methyl-1H-indazol-7-yl)piperidin-4-yl)methyl)piperazin-1-yl) pyridin-4-yl)amino)-1-methyl-2-oxo-1,2-dihydroquinolin-3-yl)oxy)-N-methylacetamide (25.4 mg) as an off-white solid.

LC-MS (ESI): m/z=795.5 [M+H]⁺.

¹H NMR (400 MHZ, DMSO-d₆) δ 10.87 (s, 1H), 9.29 (s, 1H), 8.26 (s, 1H), 8.02 (s, 1H), 7.94 (d, J=4.9 Hz, 1H), 7.59-7.49 (m, 2H), 7.47-7.36 (m, 2H), 7.27 (s, 1H), 7.05-6.96 (m, 2H), 4.57 (s, 2H), 4.33 (dd, J=9.5, 5.1 Hz, 1H), 4.24 (s, 3H), 4.09 (d, J=13.0 Hz, 2H), 3.69 (s, 3H), 3.34-3.22 (m, 2H), 3.20-2.97 (m, 5H), 2.80-2.69 (m, 2H), 2.66 (d, J=4.6 Hz, 3H), 2.65-2.57 (m, 2H), 2.39-2.24 (m, 2H), 2.21-2.10 (m, 2H), 2.05-1.92 (m, 2H), 1.88 (d, J=12.7 Hz, 2H), 1.60-1.38 (m, 2H).

### Example 93. Synthesis of 2-((6-((5-chloro-2-(4-((1-(3-(2,6-dioxopiperidin-3-yl)-1-methyl-1H-indazole-6-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide (Compound 321)

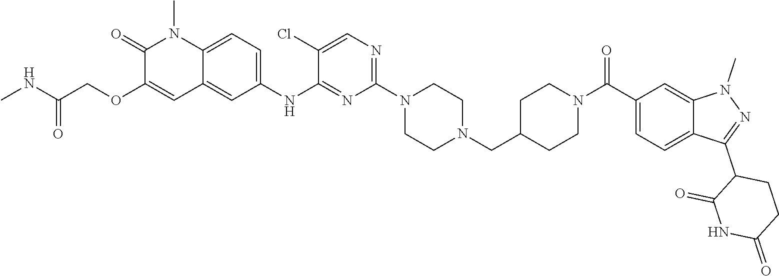 [SMILES: CNC(=O)COc1cc2cc(Nc3nc(N4CCN(CC5CCN(C(=O)c6ccc7c(C8CCC(=O)NC8=O)nn(C)c7c6)CC5)CC4)ncc3Cl)ccc2n(C)c1=O]

### Preparation of methyl 3-(2,6-bis(benzyloxy)pyridin-3-yl)-1-methyl-1H-indazole-6-carboxylate

A mixture of 3-(2,6-bis(benzyloxy)pyridin-3-yl)-6-bromo-1-methyl-1H-indazole (40.0 g, 79.9 mmol), Pd(dppf) Cl2 (11.7 g, 16.0 mmol), TEA (24.3 g, 240 mmol, 33.4 mL) in MeOH (400 mL) and DMF (120 mL) was degassed and purged with nitrogen 3 times. The reaction mixture was stirred at 70° C. for 16 hours under CO atmosphere (50.0 psi). The reaction was filtered through a pad of celite, and the celite was washed with MeOH (500 mL). The filtrate was concentrated under reduced pressure to remove MeOH. The residue was suspended in H₂O (300 mL) and extracted with EtOAc 600 mL (300 mL×2). The combined organic layers were washed with brine (300 mL), dried over anhydrous Na₂SO₄, filtered, and concentrated under reduced pressure to obtain crude product. The crude product was purified by column chromatography (SiO₂, petroleum ether/ethyl acetate=5/1 to 1/1) to afford methyl 3-(2,6-bis(benzyloxy)pyridin-3-yl)-1-methyl-1H-indazole-6-carboxylate (24.1 g) as a brown solid.

LC-MS (ESI): m/z=480.8 [M+H]⁺.

### Preparation of 3-(2,6-bis(benzyloxy)pyridin-3-yl)-1-methyl-1H-indazole-6-carboxylic acid

To a solution of methyl 3-(2,6-bis(benzyloxy)pyridin-3-yl)-1-methyl-1H-indazole-6-carboxylate (22.5 g, 46.9 mmol) in THF (230 mL), MeOH (230 mL), and H₂O (30.0 mL), and LiOH·H₂O (29.5 g, 703 mmol) was added. The mixture was stirred at 20° C. for 3 hours. The reaction mixture was concentrated under reduced pressure to give a solid. Ethyl acetate (100 mL) was added and the pH was adjusted to pH=4˜5 with 2 M HCl (50.0 mL). The reaction was filtered. The combined organic layers were washed with saturated NaCl solution (200 mL), dried over Na₂SO₄, filtered, and concentrated under reduced pressure to obtain crude product. The crude product was purified by triturated with MeOH (20.0 mL) to afford 3-(2,6-bis(benzyloxy)pyridin-3-yl)-1-methyl-1H-indazole-6-carboxylic acid (21.0 g) as a gray solid.

LC-MS (ESI): m/z=466.4 [M+H]⁺.

### Preparation of 3-(2,6-dioxopiperidin-3-yl)-1-methyl-1H-indazole-6-carboxylic acid

To a solution of 3-(2,6-bis(benzyloxy)pyridin-3-yl)-1-methyl-1H-indazole-6-carboxylic acid (20.0 g, 42.9 mmol) in THF (200 mL), EtOH (200 mL), and AcOH (20.0 mL), Pd/C (10%, 20.0 g) and Pd(OH)₂/C (10%, 20.0 g) was added under a nitrogen atmosphere. The suspension was degassed and purged with hydrogen 3 times. The mixture was stirred under a hydrogen atmosphere (50 psi) at 50° C. for 4 hours. The reaction mixture was filtered through a pad of celite, and the celite was washed with THF (1000 mL). The filtrate was concentrated under reduced pressure to obtain crude product. The crude product was triturated with EtOH 30.0 mL to afford 3-(2,6-dioxopiperidin-3-yl)-1-methyl-1H-indazole-6-carboxylic acid (13.1 g) as a white solid.

LC-MS (ESI): m/z=288.3 [M+H]⁺.

### Preparation of tert-butyl 4-((1-(3-(2,6-dioxopiperidin-3-yl)-1-methyl-1H-indazole-6-carbonyl)piperidin-4-yl)methyl)piperazine-1-carboxylate

A mixture of tert-butyl 4-(piperidin-4-ylmethyl)piperazine-1-carboxylate (191 mg, 674 μmol), 3-(2,6-dioxopiperidin-3-yl)-1-methyl-1H-indazole-6-carboxylic acid (176 mg, 613 μmol), and NMI (106 mg, 103 μL, 1.29 mmol) in DMA (3.00 mL) was treated with TCFH (206 mg, 735 μmol). The reaction mixture was allowed to stir for 3 hours. The reaction mixture was filtered and purified by reverse phase chromatography (10-60% MeCN—H₂O [+0.1% FA]) to afford tert-butyl 4-((1-(3-(2,6-dioxopiperidin-3-yl)-1-methyl-1H-indazole-6-carbonyl)piperidin-4-yl)methyl)piperazine-1-carboxylate (282 mg).

LC-MS (ESI): m/z=553.5 [M+H]⁺.

### Preparation of 3-(1-methyl-6-(4-(piperazin-1-ylmethyl)piperidine-1-carbonyl)-1H-indazol-3-yl)piperidine-2,6-dione

A mixture of tert-butyl 4-((1-(3-(2,6-dioxopiperidin-3-yl)-1-methyl-1H-indazole-6-carbonyl)piperidin-4-yl)methyl)piperazine-1-carboxylate (280 mg, 507 μmol) and DCM (2.00 mL) was treated with a mixture of DCM (2.00 mL) and TFA (2.96 g, 26.0 mmol). The reaction was stirred at room temperature for 16 hours. The volatiles were removed in vacuo to afford 3-(1-methyl-6-(4-(piperazin-1-ylmethyl)piperidine-1-carbonyl)-1H-indazol-3-yl)piperidine-2,6-dione, trifluoroacetic acid (355 mg).

### Preparation of 2-((6-((5-chloro-2-(4-((1-(3-(2,6-dioxopiperidin-3-yl)-1-methyl-1H-indazole-6-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide

A mixture of 2-((6-((2,5-dichloropyrimidin-4-yl)amino)-1-methyl-2-oxo-1,2-dihydroquinolin-3-yl)oxy)-N-methylacetamide (50.0 mg, 122 μmol), 3-(1-methyl-6-(4-(piperazin-1-ylmethyl)piperidine-1-carbonyl)-1H-indazol-3-yl)piperidine-2,6-dione (83.1 mg, 184 μmol), and DIEA (107 μL, 612 μmol) in DMSO (1.22 mL) was warmed to 80° C. for 17 hours. The reaction mixture was cooled to room temperature, filtered, and purified by prep-HPLC to afford 2-((6-((5-chloro-2-(4-((1-(3-(2,6-dioxopiperidin-3-yl)-1-methyl-1H-indazole-6-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide (32.6 mg) as a white solid.

LC-MS (ESI): m/z=824.5 [M+H]⁺.

¹H NMR (400 MHz, DMSO-d₆) δ 10.90 (s, 1H), 8.86 (s, 1H), 8.05 (s, 1H), 7.94 (t, J=4.0 Hz, 2H), 7.79-7.70 (m, 2H), 7.65 (d, J=1.1 Hz, 1H), 7.47 (d, J=9.2 Hz, 1H), 7.12 (s, 1H), 7.09 (dd, J=8.3, 1.2 Hz, 1H), 4.58 (s, 2H), 4.52 (s, 1H), 4.40 (dd, J=10.1, 5.1 Hz, 1H), 4.02 (s, 3H), 3.67 (s, 3H), 3.64-3.59 (m, 5H), 3.04 (s, 1H), 2.81 (s, OH), 2.72-2.59 (m, 4H), 2.44-2.31 (m, 5H), 2.22-2.13 (m, 3H), 1.93-1.78 (m, 2H), 1.71-1.58 (m, 1H), 1.17-1.04 (m, 2H).

### Example 94. Synthesis of 2-((6-((5-chloro-2-(4-((4-(3-(2,6-dioxopiperidin-3-yl)-1-methyl-1H-indazole-6-carbonyl)piperazin-1-yl)methyl)piperidin-1-yl)pyrimidin-4-yl)amino)-1-methyl-2-oxo-1,2-dihydroquinolin-3-yl)oxy)-N-methylacetamide (Compound 322)

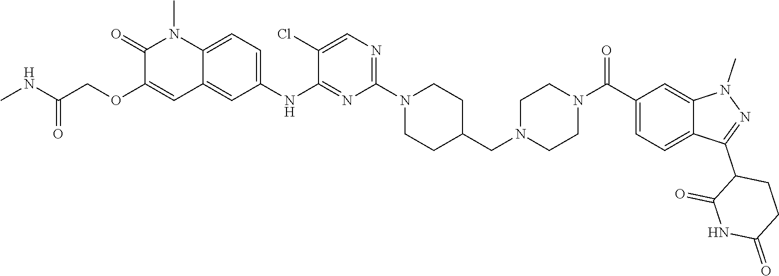 [SMILES: CNC(=O)COc1cc2cc(Nc3nc(N4CCC(CN5CCN(C(=O)c6ccc7c(C8CCC(=O)NC8=O)nn(C)c7c6)CC5)CC4)ncc3Cl)ccc2n(C)c1=O]

### Preparation of 2-((6-((5-chloro-2-(4-((4-(3-(2,6-dioxopiperidin-3-yl)-1-methyl-1H-indazole-6-carbonyl)piperazin-1-yl)methyl)piperidin-1-yl)pyrimidin-4-yl)amino)-1-methyl-2-oxo-1,2-dihydroquinolin-3-yl)oxy)-N-methylacetamide

This compound was prepared in a manner analogous to that described for 2-((6-((5-chloro-2-(4-((1-(3-(2,6-dioxopiperidin-3-yl)-1-methyl-1H-indazole-6-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide (31.9 mg)(see, e.g., Example 93).

LC-MS (ESI): m/z=824.6 [M+H]⁺.

¹H NMR (400 MHZ, DMSO-d₆) δ 10.90 (s, 1H), 8.81 (s, 1H), 8.03 (s, 1H), 7.95 (d, J=4.7 Hz, OH), 7.92 (d, J=2.4 Hz, 1H), 7.82-7.71 (m, 2H), 7.67 (s, 1H), 7.47 (d, J=9.2 Hz, 1H), 7.10 (d, J=4.3 Hz, 2H), 4.57 (s, 2H), 4.48 (d, J=12.7 Hz, 2H), 4.41 (dd, J=10.1, 5.1 Hz, 1H), 4.03 (s, 3H), 3.68 (s, 3H), 3.65-3.59 (m, 2H), 2.83 (t, J=12.4 Hz, 2H), 2.73-2.58 (m, 5H), 2.54 (s, 2H), 2.46-2.23 (m, 5H), 2.23-2.13 (m, 3H), 1.81-1.69 (m, 3H), 1.09-0.95 (m, 2H).

### Example 95. Synthesis of 2-((6-((5-chloro-2-(4-((1-(3-(2,6-dioxopiperidin-3-yl)-1-methyl-1H-indazole-7-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide (Compound 323)

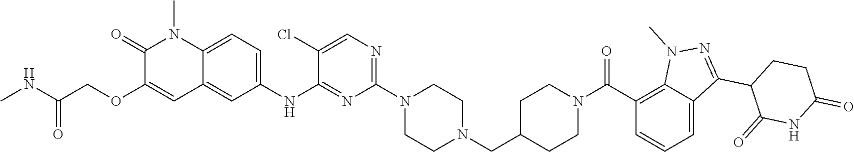 [SMILES: CNC(=O)COc1cc2cc(Nc3nc(N4CCN(CC5CCN(C(=O)c6cccc7c(C8CCC(=O)NC8=O)nn(C)c67)CC5)CC4)ncc3Cl)ccc2n(C)c1=O]

### Preparation of 2-((6-((5-chloro-2-(4-((1-(3-(2,6-dioxopiperidin-3-yl)-1-methyl-1H-indazole-7-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide

This compound was prepared in a manner analogous to that described for 2-((6-((5-chloro-2-(4-((1-(3-(2,6-dioxopiperidin-3-yl)-1-methyl-1H-indazole-6-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide (39.4 mg)(see, e.g., Example 93).

LC-MS (ESI): m/z=824.6 [M+H]⁺.

¹H NMR (400 MHZ, DMSO-d₆) δ 10.91 (s, 1H), 8.85 (s, 1H), 8.05 (s, 1H), 7.98-7.88 (m, 2H), 7.79 (t, J=7.9 Hz, 1H), 7.73 (dd, J=9.1, 2.4 Hz, 1H), 7.47 (d, J=9.1 Hz, 1H), 7.27 (dd, J=14.0, 6.9 Hz, 1H), 7.16 (t, J=7.6 Hz, 1H), 7.11 (s, 1H), 4.68-4.60 (m, 2H), 4.58 (s, 2H), 4.42 (dd, J=10.1, 5.1 Hz, 1H), 3.89 (s, 3H), 3.67 (s, 3H), 3.65-3.55 (m, 5H), 2.95-2.81 (m, 1H), 2.68-2.62 (m, 4H), 2.45-2.29 (m, 5H), 2.27-2.12 (m, 3H), 1.94-1.83 (m, 2H), 1.70-1.54 (m, 1H), 1.20-1.03 (m, 2H), 1.00-0.88 (m, 1H).

### Example 96. Synthesis of 2-((6-((5-chloro-2-(4-((4-(3-(2,6-dioxopiperidin-3-yl)-1-methyl-1H-indazole-7-carbonyl)piperazin-1-yl)methyl)piperidin-1-yl)pyrimidin-4-yl)amino)-1-methyl-2-oxo-1,2-dihydroquinolin-3-yl)oxy)-N-methylacetamide (Compound 324)

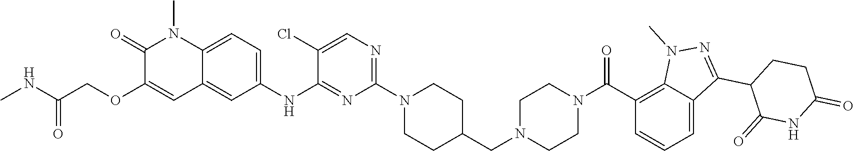 [SMILES: CNC(=O)COc1cc2cc(Nc3nc(N4CCC(CN5CCN(C(=O)c6cccc7c(C8CCC(=O)NC8=O)nn(C)c67)CC5)CC4)ncc3Cl)ccc2n(C)c1=O]

### Preparation of 2-((6-((5-chloro-2-(4-((4-(3-(2,6-dioxopiperidin-3-yl)-1-methyl-1H-indazole-7-carbonyl)piperazin-1-yl)methyl)piperidin-1-yl)pyrimidin-4-yl)amino)-1-methyl-2-oxo-1,2-dihydroquinolin-3-yl)oxy)-N-methylacetamide

This compound was prepared in a manner analogous to that described for 2-((6-((5-chloro-2-(4-((1-(3-(2,6-dioxopiperidin-3-yl)-1-methyl-1H-indazole-6-carbonyl)piperidin-4-yl)methyl)piperazin-1-yl)pyrimidin-4-yl)amino)-1-methyl-2-oxo-1,2-dihydroquinolin-3-yl)oxy)-N-methylacetamide (40.4 mg)(see, e.g., Example 93).

LC-MS (ESI): m/z=824.6 [M+H]⁺.

¹H NMR (400 MHZ, DMSO-d₆): δ 10.91 (s, 1H), 8.81 (s, 1H), 8.03 (s, 1H), 7.97-7.90 (m, 2H), 7.83-7.77 (m, OH), 7.75 (dd, J=9.1, 2.4 Hz, 1H), 7.47 (d, J=9.1 Hz, 1H), 7.28 (d, J=6.9 Hz, 1H), 7.16 (t, J=7.6 Hz, 1H), 7.10 (s, 1H), 4.57 (s, 2H), 4.47 (d, J=12.9 Hz, 2H), 4.42 (dd, J=10.1, 5.1 Hz, 1H), 3.90 (s, 3H), 3.86-3.70 (m, 2H), 3.68 (s, 3H), 2.82 (t, J=12.4 Hz, 2H), 2.71-2.62 (m, 4H), 2.54 (s, 1H), 2.44 (s, 2H), 2.33 (d, J=1.9 Hz, 3H), 2.28-2.13 (m, 4H), 1.85-1.64 (m, 4H), 1.09-0.95 (m, 2H).

### Example 97. Synthesis of 2-((6-((5-chloro-2-(5-((3-(2,6-dioxopiperidin-3-yl)-1-methyl-1H-indazol-7-yl)amino) octahydro-2H-isoindol-2-yl)pyrimidin-4-yl)amino)-1-methyl-2-oxo-1,2-dihydroquinolin-3-yl)oxy)-N-methylacetamide (Compound 339)

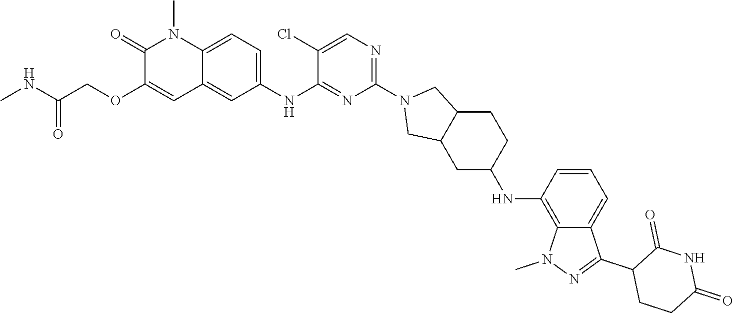 [SMILES: CNC(=O)COc1cc2cc(Nc3nc(N4CC5CCC(Nc6cccc7c(C8CCC(=O)NC8=O)nn(C)c67)CC5C4)ncc3Cl)ccc2n(C)c1=O]

### Preparation of 2-((6-((5-chloro-2-(5-((3-(2,6-dioxopiperidin-3-yl)-1-methyl-1H-indazol-7-yl)amino) octahydro-2H-isoindol-2-yl)pyrimidin-4-yl)amino)-1-methyl-2-oxo-1,2-dihydroquinolin-3-yl)oxy)-N-methylacetamide

2-((6-((5-chloro-2-(5-((3-(2,6-dioxopiperidin-3-yl)-1-methyl-1H-indazol-7-yl)amino) octahydro-2H-isoindol-2-yl)pyrimidin-4-yl)amino)-1-methyl-2-oxo-1,2-dihydroquinolin-3-yl)oxy)-N-methylacetamide (0.050 g) was prepared as an off-white solid in a similar manner as 2-((6-((5-chloro-2-((((3S)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (see, e.g., Example 52).

LC-MS (ESI): m/z=753.4 [M+H]⁺.

¹H NMR (400 MHZ, DMSO-d₆): δ 10.83 (s, 1H), 8.74-8.73 (m, 1H), 8.46 (br s, 1H), 8.17 (br s, 1H), 8.04-7.8 (m, 4H), 7.48-7.43 (m, 1H), 7.12-7.10 (m, 1H), 6.90-6.80 (m, 2H), 6.53 (d, J=8.00 Hz, 1H), 6.15 (s, 1H), 4.91-4.89 (m, 1H), 4.56 (br s, 2H), 4.25-4.18 (m, 4H), 3.67-3.56 (m, 3H), 3.51-3.31 (m, 3H), 2.66-2.56 (m, 5H), 2.49-2.32 (m, 1H), 2.27-2.14 (m, 1H), 2.13-1.79 (m, 4H), 1.69-1.34 (m, 3H).

### Example 98. Synthesis of 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-methyl-2-oxo-1,2-dihydroquinolin-3-yl)oxy)-N-methylacetamide (Compound 341)

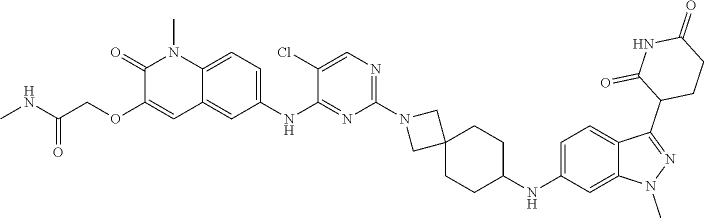 [SMILES: CNC(=O)COc1cc2cc(Nc3nc(N4CC5(CCC(Nc6ccc7c(C8CCC(=O)NC8=O)nn(C)c7c6)CC5)C4)ncc3Cl)ccc2n(C)c1=O]

### Preparation of tert-butyl 7-((3-(2,6-bis(benzyloxy)pyridin-3-yl)-1-methyl-1H-indazol-6-yl)amino)-2-azaspiro[3.5]nonane-2-carboxylate

A stirred solution of 3-(2,6-bis(benzyloxy)pyridin-3-yl)-6-bromo-1-methyl-1H-indazole (0.80 g, 1.60 mmol), tert-butyl 7-amino-2-azaspiro[3.5]nonane-2-carboxylate (0.58 g, 2.40 mmol), and KOtBu (0.54 g, 4.80 mmol) in 1,4-dioxane (20 mL) was degassed with argon for 10 minutes. Brettphos Pd G3 (0.14 g, 0.16 mmol) was added, and the reaction was heated to 80° C. for 2 hours. The reaction mixture was filtered through a pad of celite, and the celite was washed with ethyl acetate (100 mL). The filtrate was concentrated under vacuum to obtain crude product. The crude product was purified by flash column chromatography (SiO₂, 230-400, 40% ethyl acetate in petroleum ether) to afford tert-butyl 7-((3-(2,6-bis(benzyloxy)pyridin-3-yl)-1-methyl-1H-indazol-6-yl)amino)-2-azaspiro[3.5]nonane-2-carboxylate (0.90 g) as an off-white solid.

LC-MS (ESI): m/z=661.6 [M+2]⁺.

### Preparation of tert-butyl 7-((3-(2,6-dioxopiperidin-3-yl)-1-methyl-1H-indazol-6-yl)amino)-2-azaspiro[3.5]nonane-2-carboxylate

To a stirred solution of tert-butyl 7-((3-(2,6-bis(benzyloxy)pyridin-3-yl)-1-methyl-1H-indazol-6-yl)amino)-2-azaspiro[3.5]nonane-2-carboxylate (0.90 g, 1.36 mmol) in THF (27.0 mL), 20% Pd(OH)₂ (0.90 g) was added. The reaction was put under a hydrogen atmosphere (70 psi) and stirred at room temperature for 16 hours. The reaction mixture was filtered through a pad of celite, and the celite was washed with ethyl acetate (200 mL). The filtrate was concentrated under reduced pressure to afford tert-butyl 7-((3-(2,6-dioxopiperidin-3-yl)-1-methyl-1H-indazol-6-yl)amino)-2-azaspiro[3.5]nonane-2-carboxylate (0.70 g) as a brown solid.

LC-MS (ESI): m/z=482.57 [M+H]⁺.

### Preparation of 3-(6-((2-azaspiro[3.5]nonan-7-yl)amino)-1-methyl-1H-indazol-3-yl)piperidine-2,6-dione

A stirred solution of tert-butyl 7-((3-(2,6-dioxopiperidin-3-yl)-1-methyl-1H-indazol-6-yl)amino)-2-azaspiro[3.5]nonane-2-carboxylate (0.70 g, 1.45 mmol) in DCM (14.0 mL) was cooled to 0° C. TFA (7.0 mL) was added, and the reaction was allowed to warm to room temperature over 3 hours with stirring. The reaction mixture was concentrated under vacuum to obtain crude product. The crude product was triturated with diethyl ether (30 mL) to afford 3-(6-((2-azaspiro[3.5]nonan-7-yl)amino)-1-methyl-1H-indazol-3-yl)piperidine-2,6-dione (0.9 g) as an off-white solid.

LC-MS (ESI): m/z=382.4 [M+H]⁺.

### Preparation of 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-methyl-2-oxo-1,2-dihydroquinolin-3-yl)oxy)-N-methylacetamide

To a stirred solution of 3-(6-((2-azaspiro[3.5]nonan-7-yl)amino)-1-methyl-1H-indazol-3-yl)piperidine-2,6-dione (0.25 g, 0.66 mmol) and 2-((6-((2,5-dichloropyrimidin-4-yl)amino)-1-methyl-2-oxo-1,2-dihydroquinolin-3-yl)oxy)-N-methylacetamide (0.11 g, 0.26 mmol) in DMSO (5.0 mL), N,N-diisopropylethylamine (0.90 mL, 5.25 mmol) was added. The reaction mixture was stirred at 100° C. for 2 hours. Reaction solvent was removed using a centrifugal evaporator to obtain crude product. The crude product was purified by prep-HPLC to afford 2-((6-((5-chloro-2-(7-((3-(2,6-dioxopiperidin-3-yl)-1-methyl-1H-indazol-6-yl)amino)-2-azaspiro[3.5]nonan-2-yl)pyrimidin-4-yl)amino)-1-methyl-2-oxo-1,2-dihydroquinolin-3-yl)oxy)-N-methylacetamide (0.119 mg) as an off-white solid.

LC-MS (ESI): m/z=753.53 [M+H]⁺.

¹H NMR (400 MHZ, DMSO-d₆): δ 10.82 (s, 1H), 8.80 (s, 1H), 8.54 (s, 1H), 8.03 (s, 1H), 7.99 (d, J=4.40 Hz, 1H), 7.87 (dd, J=2.80, 9.00 Hz, 1H), 7.47 (d, J=9.20 Hz, 1H), 7.31 (d, J=8.40 Hz, 1H), 7.15 (s, 1H), 6.52 (dd, J=1.20, 8.80 Hz, 1H), 6.39 (s, 1H), 5.72 (d, J=8.00 Hz, 1H), 4.19 (s, 2H), 4.17-4.15 (m, 1H), 3.81 (s, 3H), 3.75 (s, 2H), 3.70-3.68 (m, 5H), 3.32-3.17 (m, 1H), 2.67 (d, J=2.40 Hz, 3H), 2.60-2.58 (m, 2H), 2.49-2.30 (m, 1H), 2.30-2.28 (m, 1H), 1.94 (d, J=10.40 Hz, 4H), 1.66 (t, J=10.80 Hz, 2H), 1.26-1.23 (m, 2H).

### Example 99. Synthesis of 2-((6-((5-chloro-2-(7-((3-(2,6-dioxopiperidin-3-yl)-1-methyl-1H-indazol-7-yl)amino)-2-azaspiro[3.5]nonan-2-yl)pyrimidin-4-yl)amino)-1-methyl-2-oxo-1,2-dihydroquinolin-3-yl)oxy)-N-methylacetamide (Compound 342)

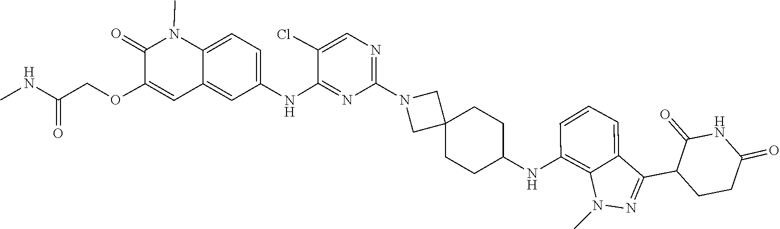 [SMILES: CNC(=O)COc1cc2cc(Nc3nc(N4CC5(CCC(Nc6cccc7c(C8CCC(=O)NC8=O)nn(C)c67)CC5)C4)ncc3Cl)ccc2n(C)c1=O]

### Preparation of 2-((6-((5-chloro-2-(7-((3-(2,6-dioxopiperidin-3-yl)-1-methyl-1H-indazol-7-yl)amino)-2-azaspiro[3.5]nonan-2-yl)pyrimidin-4-yl)amino)-1-methyl-2-oxo-1,2-dihydroquinolin-3-yl)oxy)-N-methylacetamide

2-((6-((5-chloro-2-(7-((3-(2,6-dioxopiperidin-3-yl)-1-methyl-1H-indazol-7-yl)amino)-2-azaspiro[3.5]nonan-2-yl)pyrimidin-4-yl)amino)-1-methyl-2-oxo-1,2-dihydroquinolin-3-yl)oxy)-N-methylacetamide (0.107 g) was prepared as an off-white solid in a similar manner as 2-((6-((5-chloro-2-((((3S)-1-(3-(2,6-dioxopiperidin-3-yl)-1-methyl-1H-indazol-7-yl)piperidin-3-yl)methyl)amino)pyrimidin-4-yl)amino)-1-methyl-2-oxo-1,2-dihydroquinolin-3-yl)oxy)-N-methylacetamide (see, e.g., Example 52).

LC-MS (ESI): m/z=753.50 [M+H]⁺.

¹H NMR (400 MHZ, DMSO-d₆): δ 10.80 (s, 1H), 8.79 (s, 1H), 8.14 (br, 1H), 8.03 (s, 1H), 7.98-7.97 (m, 1H), 7.86 (d, J=7.60 Hz, 1H), 7.47 (d, J=9.20 Hz, 1H), 7.12 (s, 1H), 6.97 (d, J=8.00 Hz, 1H), 6.89 (t, J=8.00 Hz, 1H), 6.54 (d, J=7.60 Hz, 1H), 4.95 (s, 1H), 4.61 (s, 2H), 4.24-4.23 (m, 4H), 3.75 (s, 2H), 3.71-3.68 (m, 5H), 3.32 (br s, 1H), 2.67-2.67 (m, 5H), 2.27-2.26 (m, 1H), 2.17 (d, J=5.60 Hz, 1H), 1.93 (d, J=13.20 Hz, 4H), 1.65 (t, J=10.80 Hz, 2H), 1.48 (s, 2H).

### Example 100. Synthesis of 2-((6-((5-chloro-2-(8-((3-(2,6-dioxopiperidin-3-yl)-1-methyl-1H-indazol-6-yl)amino)-2-azaspiro[4.5]decan-2-yl)pyrimidin-4-yl)amino)-1-methyl-2-oxo-1,2-dihydroquinolin-3-yl)oxy)-N-methylacetamide (Compound 343)

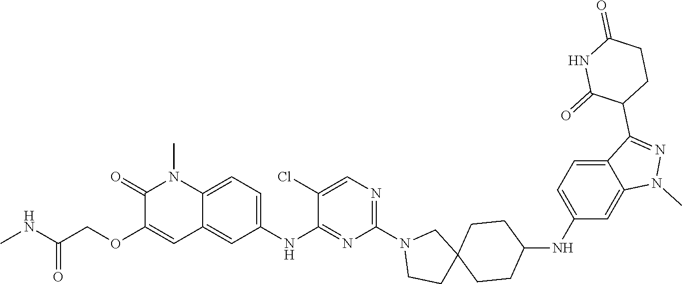

### Preparation of tert-butyl 8-((3-(2,6-bis(benzyloxy)pyridin-3-yl)-1-methyl-1H-indazol-6-yl)amino)-2-azaspiro[4.5]decane-2-carboxylate

To a stirred solution of 3-(2,6-bis(benzyloxy)pyridin-3-yl)-6-bromo-1-methyl-1H-indazole (0.80 g, 1.60 mmol) in dioxane (16.0 mL), tert-butyl 8-amino-2-azaspiro[4,5]decane-2-carboxylate (0.61 g, 2.40 mmol) and cesium carbonate (1.56 g, 4.80 mmol) were added. The reaction was degassed with argon for 5 minutes. RuPhos (0.07 g, 0.16 mmol) and RuPhos PdG3 (0.07 g, 0.08 mmol) were added, and the reaction was heated to 100° C. for 18 hours. The reaction mixture was diluted with DCM (50 mL) and filtered through a celite pad. The filtrate was concentrated to obtain crude product. The crude product was purified by column chromatography (SiO₂, 230-400, product was eluted at 21% ethyl acetate in petroleum ether) to afford tert-butyl 8-((3-(2,6-bis(benzyloxy)pyridin-3-yl)-1-methyl-1H-indazol-6-yl)amino)-2-azaspiro[4.5]decane-2-carboxylate (0.85 g) as a pale brown semi-solid.

LC-MS (ESI): m/z=674.66 [M+H]⁺.

### Preparation of tert-butyl 8-((3-(2,6-dioxopiperidin-3-yl)-1-methyl-1H-indazol-6-yl)amino)-2-azaspiro[4.5]decane-2-carboxylate

To a stirred solution of tert-butyl 8-((3-(2,6-bis(benzyloxy)pyridin-3-yl)-1-methyl-1H-indazol-6-yl)amino)-2-azaspiro[4.5]decane-2-carboxylate (0.85 g, 1.26 mmol) in THF (102 mL), 20% Pd(OH)₂/C (0.85 g, 100% w/w) was added. The reaction mixture was put under H₂ atmosphere (80 psi) and stirred at room temperature for 16 hours. The reaction mixture was diluted with DCM (10 mL) and filtered through a celite bed. The celite was washed with DCM:THF (1:1, 500 mL). The filtrate was concentrated to obtain tert-butyl 8-((3-(2,6-dioxopiperidin-3-yl)-1-methyl-1H-indazol-6-yl)amino)-2-azaspiro[4.5]decane-2-carboxylate (0.92 g) as pale brown semi solid.

LC-MS (ESI): m/z=469.65 [M+H]⁺.

### Preparation of 3-(6-((2-azaspiro[4.5]decan-8-yl)amino)-1-methyl-1H-indazol-3-yl) piperidine-2,6-dione

To a stirred solution tert-butyl 8-((3-(2,6-dioxopiperidin-3-yl)-1-methyl-1H-indazol-6-yl)amino)-2-azaspiro[4.5]decane-2-carboxylate (0.92 g, 1.86 mmol) in DCM (10.0 mL), trifluoro acetic acid (7.0 mL) was added. The reaction was stirred at room temperature for 3 hours. The reaction mixture was concentrated under vacuum to obtain 3-(6-((2-azaspiro[4.5]decan-8-yl)amino)-1-methyl-1H-indazol-3-yl)piperidine-2,6-dione (0.82 g) as a pale green semi-solid.

LC-MS (ESI): m/z=396.55 [M+H]⁺.

### Preparation of 2-((6-((5-chloro-2-(8-((3-(2,6-dioxopiperidin-3-yl)-1-methyl-1H-indazol-6-yl)amino)-2-azaspiro[4.5]decan-2-yl)pyrimidin-4-yl)amino)-1-methyl-2-oxo-1,2-dihydroquinolin-3-yl)oxy)-N-methylacetamide

To a stirred solution of 3-(6-((2-azaspiro[4.5]decan-8-yl)amino)-1-methyl-1H-indazol-3-yl)piperidine-2,6-dione (0.3 g, 0.76 mmol) in DMSO (3.0 mL), 2-((6-((2,5-dichloropyrimidin-4-yl)amino)-1-methyl-2-oxo-1,2-dihydroquinolin-3-yl)oxy)-N-methylacetamide (0.15 g, 0.38 mmol) and DIPEA (3.0 mL) were added. The reaction was heated at 100° C. for 16 hours. Reaction solvent was removed using a centrifugal evaporator to obtain crude product. The crude product was purified by prep-HPLC to afford 2-((6-((5-chloro-2-(8-((3-(2,6-dioxopiperidin-3-yl)-1-methyl-1H-indazol-6-yl)amino)-2-azaspiro[4.5]decan-2-yl)pyrimidin-4-yl)amino)-1-methyl-2-oxo-1,2-dihydroquinolin-3-yl)oxy)-N-methylacetamide (0.088 g) as a pale brown solid.

LC-MS (ESI): m/z=767.52 [M+H]⁺.

¹H NMR data: (400 MHz, DMSO-d₆): 10.82 (s, 1H), 8.75 (s, 1H), 8.09-7.89 (m, 4H), 7.47 (d, J=8.80 Hz, 1H), 7.31 (d, J=8.80 Hz, 1H), 7.15 (s, 1H), 6.50-6.49 (m, 1H), 6.38 (br s, 1H), 5.77-5.75 (m, 1H), 4.57-4.52 (m, 2H), 4.17-4.15 (m, 1H), 3.80 (d, J=6.40 Hz, 3H), 3.68 (s, 3H), 3.53-3.51 (m, 2H), 3.50-3.41 (m, 2H), 2.61-2.60 (m, 5H), 2.34-2.32 (m, 1H), 2.16-2.15 (m, 1H), 1.93-1.90 (m, 3H), 1.89-1.88 (m, 1H), 1.80-1.79 (m, 3H), 1.69-1.66 (m, 2H), 1.57-1.23 (m, 3H).

### Example 101. Synthesis of 2-((6-((5-chloro-2-(8-((3-(2,6-dioxopiperidin-3-yl)-1-methyl-1H-indazol-7-yl)amino)-2-azaspiro[4.5]decan-2-yl)pyrimidin-4-yl)amino)-1-methyl-2-oxo-1,2-dihydroquinolin-3-yl)oxy)-N-methylacetamide (Compound 344)

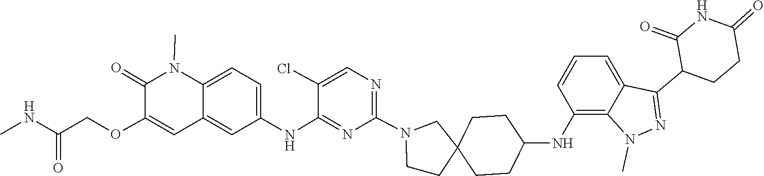 [SMILES: CNC(=O)COc1cc2cc(Nc3nc(N4CCC5(CCC(Nc6cccc7c(C8CCC(=O)NC8=O)nn(C)c67)CC5)C4)ncc3Cl)ccc2n(C)c1=O]

### Preparation of tert-butyl 8-((3-(2,6-bis(benzyloxy)pyridin-3-yl)-1-methyl-1H-indazol-7-yl)amino)-2-azaspiro[4.5]decane-2-carboxylate

To a stirred solution of 3-(2,6-bis(benzyloxy)pyridin-3-yl)-7-bromo-1-methyl-1H-indazole (0.80 g, 1.60 mmol) in dioxane (10 mL) was added tert-butyl 8-amino-2-azaspiro[4.5]decane-2-carboxylate (0.61 mg, 2.40 mmol) and cesium carbonate (1.56 g, 4.80 mmol). The reaction was degassed with argon for 5 minutes, Pd-PEPPSI-iHeptCl (0.08 g, 0.08 mmol) was added, and the reaction was heated to 80° C. for 16 hours. The reaction mixture was filtered through a pad of celite, and the celite was washed with ethyl acetate (100 mL). The collected filtrate was concentrated under vacuum to obtain crude product. The crude product was purified by column chromatography (SiO₂, 100-200 mesh; 27% EtOAc in petroleum ether) to afford tert-butyl 8-((3-(2,6-bis(benzyloxy)pyridin-3-yl)-1-methyl-1H-indazol-7-yl)amino)-2-azaspiro[4.5]decane-2-carboxylate (0.81 g) as a pale yellow semisolid.

LC-MS (ESI): m/z=674.62 [M+H]⁺.

### Preparation of tert-butyl 8-((3-(2,6-dioxopiperidin-3-yl)-1-methyl-1H-indazol-7-yl)amino)-2-azaspiro[4.5]decane-2-carboxylate

To a stirred solution of tert-butyl 8-((3-(2,6-bis(benzyloxy)pyridin-3-yl)-1-methyl-1H-indazol-7-yl)amino)-2-azaspiro[4.5]decane-2-carboxylate (0.80 g, 1.19 mmol) in THF (104.0 mL) was added 20% Pd(OH)₂/C (0.80 g, 200% w/w). The reaction was put under H₂ atmosphere (80 psi) and stirred for 16 hours. The reaction mixture was filtered through a pad of celite and the celite was washed with 20% THF in DCM (300 mL). The collected filtrate was concentrated under vacuum to obtain crude product. The crude was washed with n-pentane (20 mL) and dried under vacuum to afford tert-butyl 8-((3-(2,6-dioxopiperidin-3-yl)-1-methyl-1H-indazol-7-yl)amino)-2-azaspiro[4.5]decane-2-carboxylate (0.71 g) as a brown semi solid.

LC-MS (ESI): m/z=496.08 [M+H]⁺.

### Preparation of 3-(7-((2-azaspiro[4.5]decan-8-yl)amino)-1-methyl-1H-indazol-3-yl)piperidine-2,6-dione

To a stirred solution of tert-butyl 8-((3-(2,6-dioxopiperidin-3-yl)-1-methyl-1H-indazol-7-yl)amino)-2-azaspiro[4.5]decane-2-carboxylate (0.70 g, 1.41 mmol) in DCM (7 mL), trifluoro acetic acid (5.0 mL) was added at 0° C. under nitrogen atmosphere. The reaction was stirred at room temperature for 2 hours. Solvent was removed and the crude product was washed with n-pentane (20 mL) and dried under vacuum to afford 3-(7-((2-azaspiro[4.5]decan-8-yl)amino)-1-methyl-1H-indazol-3-yl)piperidine-2,6-dione (0.80 g) as a brown semi solid.

LC-MS (ESI): m/z=396.52 [M+H]⁺.

### Preparation of 2-((6-((5-chloro-2-(8-((3-(2,6-dioxopiperidin-3-yl)-1-methyl-1H-indazol-7-yl)amino)-2-azaspiro[4.5]decan-2-yl)pyrimidin-4-yl)amino)-1-methyl-2-oxo-1,2-dihydroquinolin-3-yl)oxy)-N-methylacetamide

To a stirred solution of 3-(7-((2-azaspiro[4.5]decan-8-yl)amino)-1-methyl-1H-indazol-3-yl)piperidine-2,6-dione (0.40 g, 1.01 mmol) in DMSO (4 mL), 2-((6-((2,5-dichloropyrimidin-4-yl)amino)-1-methyl-2-oxo-1,2-dihydroquinolin-3-yl)oxy)-N-methylacetamide (0.20 g, 0.50 mmol) and DIPEA (3.52 mL, 20.2 mmol) were added. The reaction was heated at 100° C. for 8 hours. The reaction solvent was removed using a centrifugal evaporator to obtain crude product. The crude product was purified by preparative HPLC to afford 2-((6-((5-chloro-2-(8-((3-(2,6-dioxopiperidin-3-yl)-1-methyl-1H-indazol-7-yl)amino)-2-azaspiro[4.5]decan-2-yl)pyrimidin-4-yl)amino)-1-methyl-2-oxo-1,2-dihydroquinolin-3-yl)oxy)-N-methylacetamide (0.10 g) as an off-white solid.

LC-MS (ESI): m/z=767.48 [M+H]⁺.

¹H NMR (400 MHZ, DMSO-d₆): δ 10.85 (s, 1H), 8.75 (s, 1H), 8.53 (s, 1H), 8.19-7.87 (m, 4H), 7.47 (d, J=8.80 Hz, 1H), 7.15 (br s, 1H), 6.98-6.94 (m, 1H), 6.91-6.86 (m, 1H), 6.54-6.52 (m, 1H), 4.95 (d, J=5.60 Hz, 1H), 4.58 (s, 2H), 4.25 (s, 4H), 3.68 (s, 3H), 3.56-3.54 (m, 2H), 3.52 (s, 1H), 3.50-3.48 (m, 2H), 2.67-2.56 (m, 5H), 2.30-2.7 (m, 1H), 2.17-2.15 (m, 1H), 2.01-1.92 (m, 3H), 1.90-1.88 (m, 1H), 1.81-1.80 (m, 2H), 1.66-1.54 (m, 3H).

### Example 102. Synthesis of 2-((6-((5-chloro-2-(4-(1-(3-(2,6-dioxopiperidin-3-yl)-1-methyl-1H-indazol-7-yl)piperidine-4-carbonyl)piperazin-1-yl)pyrimidin-4-yl)amino)-1-methyl-2-oxo-1,2-dihydroquinolin-3-yl)oxy)-N-methylacetamide (Compound 345)

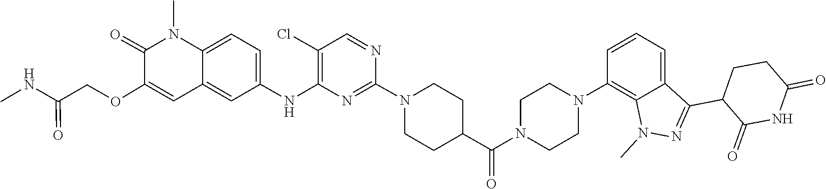 [SMILES: CNC(=O)COc1cc2cc(Nc3nc(N4CCC(C(=O)N5CCN(c6cccc7c(C8CCC(=O)NC8=O)nn(C)c67)CC5)CC4)ncc3Cl)ccc2n(C)c1=O]

### Preparation of tert-butyl 1-(3-(2,6-bis(benzyloxy)pyridin-3-yl)-1-methyl-1H-indazol-7-yl)piperidine-4-carboxylate

3-(2,6-Bis(benzyloxy)pyridin-3-yl)-7-bromo-1-methyl-1H-indazole (600 mg, 1.20 mmol), 4-piperidinecarboxylic acid t-butyl ester HCl (306 mg, 1.38 mmol), 1,4-dioxane (10 mL), and chloro(2-dicyclohexylphosphino-2′,6′-di-i-propoxy-1,1′-biphenyl)(2′-amino-1,1′-biphenyl-2-yl)palladium (II)(74.5 mg, 95.9 μmol) were combined. The reaction was purged with nitrogen for 5 minutes. Sodium tert-butoxide (461 mg, 2.40 mL, 2.0 molar, 4.80 mmol) was added, and the reaction was heated to 100° C. After heating for 5 hours, the reaction was quenched with water. The reaction was diluted with DCM and the layers separated. The organic layer was washed with sat. NaCl, dried with MgSO₄, filtered, and loaded onto directly onto silica. The crude product was purified via flash column chromatography (silica, 0-100% EtOAc in heptanes) to afford tert-butyl 1-(3-(2,6-bis(benzyloxy)pyridin-3-yl)-1-methyl-1H-indazol-7-yl)piperidine-4-carboxylate (474.7 mg).

¹H NMR (400 MHZ, DMSO-d₆) δ 7.95-7.82 (m, 1H), 7.54-7.23 (m, 12H), 7.04-6.89 (m, 2H), 6.59 (d, J=8.0 Hz, 1H), 5.43 (d, J=5.3 Hz, 4H), 4.32 (s, 3H), 3.27 (d, J=11.4 Hz, 2H), 2.72 (t, J=12.1 Hz, 2H), 2.35 (d, J=12.0 Hz, 1H), 2.02-1.93 (m, 3H), 1.78 (d, J=12.7 Hz, 2H), 1.44 (s, 9H).

### Preparation of tert-butyl 1-(3-(2,6-dioxopiperidin-3-yl)-1-methyl-1H-indazol-7-yl)piperidine-4-carboxylate

Tert-butyl 1-(3-(2,6-bis(benzyloxy)pyridin-3-yl)-1-methyl-1H-indazol-7-yl)piperidine-4-carboxylate (475 mg, 707 μmol) was dissolved in EtOAc (120 mL). The reaction was flushed with nitrogen for 5 minutes and then palladium on carbon (75.2 mg) was added, and the reaction was put under hydrogen via balloon and heated to 60° C. for 18 hours. The reaction was filtered reaction through celite and concentrated to afford tert-butyl 1-(3-(2,6-dioxopiperidin-3-yl)-1-methyl-1H-indazol-7-yl)piperidine-4-carboxylate (308 mg).

¹H NMR (400 MHZ, DMSO-d₆) δ 10.88 (s, 1H), 7.43-7.34 (m, 1H), 7.06-6.97 (m, 2H), 4.36 (ddd, J=19.9, 9.6, 5.1 Hz, 1H), 4.24 (s, 3H), 3.25 (s, 2H), 2.74-2.55 (m, 2H), 2.34 (ddt, J=13.8, 9.3, 4.6 Hz, 1H), 2.24-2.07 (m, 1H), 1.94 (d, J=15.6 Hz, 2H), 1.77 (d, J=12.7 Hz, 1H), 1.44 (s, 9H).

### Preparation of 1-(3-(2,6-dioxopiperidin-3-yl)-1-methyl-1H-indazol-7-yl)piperidine-4-carboxylic acid

Tert-butyl 1-(3-(2,6-dioxopiperidin-3-yl)-1-methyl-1H-indazol-7-yl)piperidine-4-carboxylate (308 mg, 651 μmol) was dissolved in DCM (6 mL). Trifluoroacetic acid (0.496 mL) was added and the reaction was stirred at room temperature. After 3 hours, the reaction was concentrated via rotovap. The crude product was diluted reaction with DMSO (3 mL), filtered through a syringe filter, and purified via reverse phase chromatography to afford 1-(3-(2,6-dioxopiperidin-3-yl)-1-methyl-1H-indazol-7-yl)piperidine-4-carboxylic acid (175 mg).

LC-MS (ESI): m/z=371.5 [M+H]⁺.

### Preparation of tert-butyl 4-(1-(3-(2,6-dioxopiperidin-3-yl)-1-methyl-1H-indazol-7-yl)piperidine-4-carbonyl)piperazine-1-carboxylate

1-(3-(2,6-Dioxopiperidin-3-yl)-1-methyl-1H-indazol-7-yl)piperidine-4-carboxylic acid (132 mg, 356 μmol), tert-butyl-1-piperazinecarboxylate (72.9 mg, 391 μmol), HATU (162 mg, 427 μmol), DMF (3 mL), and diisopropylethylamine (138 mg, 1.06 mmol) were combined. The reaction was stirred at room temperature for 18 hours. The reaction was filtered via syringe filter and purified via reverse phase chromatography to afford tert-butyl 4-(1-(3-(2,6-dioxopiperidin-3-yl)-1-methyl-1H-indazol-7-yl)piperidine-4-carbonyl)piperazine-1-carboxylate (158 mg).

LC-MS (ESI): m/z=539.8 [M+H]⁺.

### Preparation of 3-(1-methyl-7-(4-(piperazine-1-carbonyl)piperidin-1-yl)-1H-indazol-3-yl)piperidine-2,6-dione

Tert-butyl 4-(1-(3-(2,6-dioxopiperidin-3-yl)-1-methyl-1H-indazol-7-yl)piperidine-4-carbonyl)piperazine-1-carboxylate (158 mg, 294 μmol) was dissolved in DCM (8 mL). Trifluoroacetic acid (335 mg, 2.94 mmol) was added and the reaction was stirred for 3 hours. The reaction was concentrated to afford 3-(1-methyl-7-(4-(piperazine-1-carbonyl)piperidin-1-yl)-1H-indazol-3-yl)piperidine-2,6-dione (129 mg). The product was used in the next reaction without further purification.

LC-MS (ESI): m/z=439.4 [M+H]⁺.

### Preparation of 2-((6-((5-chloro-2-(4-(1-(3-(2,6-dioxopiperidin-3-yl)-1-methyl-1H-indazol-7-yl)piperidine-4-carbonyl)piperazin-1-yl)pyrimidin-4-yl)amino)-1-methyl-2-oxo-1,2-dihydroquinolin-3-yl)oxy)-N-methylacetamide

Diisopropylethylamine (0.19 g, 0.26 mL, 1.5 mmol), 2-((6-((2,5-dichloropyrimidin-4-yl)amino)-1-methyl-2-oxo-1,2-dihydroquinolin-3-yl)oxy)-N-methylacetamide (61 mg, 0.15 mmol), 3-(1-methyl-7-(4-(piperazine-1-carbonyl)piperidin-1-yl)-1H-indazol-3-yl)piperidine-2,6-dione (65 mg, 0.15 mmol), and DMSO (2 mL) were combined. The reaction was heated to 85° C. for 16 hours. After cooling, the reaction was filtered via syringe filter. The crude product was purified via reverse phase chromatography to afford 2-((6-((5-chloro-2-(4-(1-(3-(2,6-dioxopiperidin-3-yl)-1-methyl-1H-indazol-7-yl)piperidine-4-carbonyl)piperazin-1-yl)pyrimidin-4-yl)amino)-1-methyl-2-oxo-1,2-dihydroquinolin-3-yl)oxy)-N-methylacetamide (76.9 mg) as a white solid.

LC-MS (ESI): m/z=811.1 [M+H]⁺.

¹H NMR (400 MHZ, DMSO) δ 10.87 (s, 1H), 8.92 (s, 1H), 8.09 (s, 1H), 7.94 (dd, J=10.0, 3.7 Hz, 2H), 7.76 (dd, J=9.1, 2.4 Hz, 1H), 7.49 (d, J=9.1 Hz, 1H), 7.38 (dd, J=7.0, 1.9 Hz, 1H), 7.18 (s, 1H), 7.07-6.96 (m, 2H), 4.59 (s, 2H), 4.33 (dd, J=9.6, 5.1 Hz, 1H), 4.25 (s, 3H), 3.69 (s, 9H), 3.59-3.53 (m, 2H), 3.29-3.20 (m, 4H), 2.82 (t, J=19.3 Hz, 3H), 2.66 (s, 2H), 2.33 (dtd, J=14.4, 9.4, 5.1 Hz, 1H), 2.16 (dq, J=13.2, 5.5 Hz, 1H), 1.89 (d, J=11.6 Hz, 1H), 1.84-1.77 (m, 3H).

### Example 103. Synthesis of 3-(7-(4-(((S)-4-(5-chloro-4-((1-methyl-2-oxo-3-(2-oxopropoxy)-1,2-dihydroquinolin-6-yl)amino)pyrimidin-2-yl)-2-methylpiperazin-1-yl)methyl)piperidin-1-yl)-1-methyl-1H-indazol-3-yl)piperidine-2,6-dione (Compound 347a)

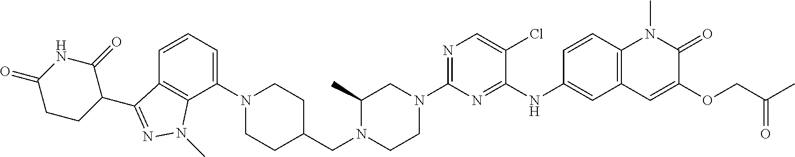 [SMILES: CC(=O)COc1cc2cc(Nc3nc(N4CCN(CC5CCN(c6cccc7c(C8CCC(=O)NC8=O)nn(C)c67)CC5)[C@@H](C)C4)ncc3Cl)ccc2n(C)c1=O]

### Preparation of 3-(7-(4-(((S)-4-(5-chloro-4-((1-methyl-2-oxo-3-(2-oxopropoxy)-1,2-dihydroquinolin-6-yl)amino)pyrimidin-2-yl)-2-methylpiperazin-1-yl)methyl)piperidin-1-yl)-1-methyl-1H-indazol-3-yl)piperidine-2,6-dione

To a stirred solution of 3-(1-methyl-7-(4-(((S)-2-methylpiperazin-1-yl)methyl)piperidin-1-yl)-1H-indazol-3-yl)piperidine-2,6-dione (0.20 g, 0.45 mmol)(see, e.g., Example 69) in DMSO (4 mL), 6-((2,5-dichloropyrimidin-4-yl)amino)-1-methyl-3-(2-oxopropoxy) quinolin-2 (1H)-one (0.086 g, 0.22 mmol)(see, e.g., International Publication No. WO 2021/077010) and DIPEA (0.78 mL, 4.50 mmol) were added. The reaction was heated at 100° C. for 12 hours. The reaction mixture was added to ice water and stirred for 30 minutes to precipitate solid. The precipitate was filtered and dried to obtain crude product. The crude product was purified by prep HPLC to afford 3-(7-(4-(((S)-4-(5-chloro-4-((1-methyl-2-oxo-3-(2-oxopropoxy)-1,2-dihydroquinolin-6-yl)amino)pyrimidin-2-yl)-2-methylpiperazin-1-yl)methyl)piperidin-1-yl)-1-methyl-1H-indazol-3-yl)piperidine-2,6-dione (50 mg) as an off-white solid.

LC-MS (ESI): m/z=795.47 [M+H]⁺.

¹H NMR (400 MHZ, DMSO-d₆): δ 10.88 (s, 1H), 9.06-9.01 (m, 2H), 8.14 (d, J=8.40 Hz, 1H), 7.87 (d, J=8.40 Hz, 1H), 7.68 (d, J=8.80 Hz, 1H), 7.47 (d, J=9.20 Hz, 1H), 7.40 (d, J=8.40 Hz, 1H), 7.02-6.95 (m, 3H), 4.90 (s, 2H), 4.48 (s, 1H), 4.33-4.32 (m, 1H), 4.26 (s, 3H), 3.84 (s, 4H), 3.04-2.98 (m, 8H), 2.65-2.63 (m, 1H), 2.51 (br, 3H), 2.33 (s, 1H), 2.16-2.14 (m, 4H), 2.01-1.99 (m, 2H), 1.75 (s, 1H), 1.59-1.56 (m, 2H), 1.55-1.35 (m, 3H).

### Example 104. Synthesis of 3-(6-(4-((9-(5-chloro-4-((1-methyl-2-oxo-3-(2-oxopropoxy)-1,2-dihydroquinolin-6-yl)amino)pyrimidin-2-yl)-3,9-diazaspiro[5.5]undecan-3-yl)methyl)piperidin-1-yl)-1-methyl-1H-indazol-3-yl)piperidine-2,6-dione (Compound 348)

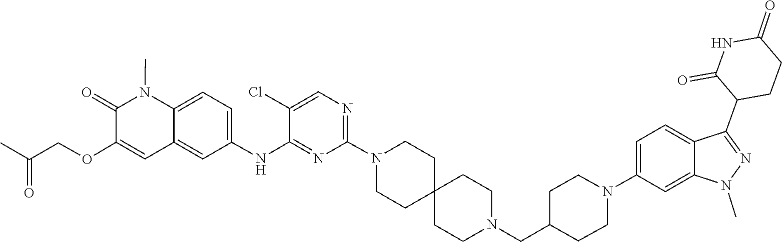 [SMILES: CC(=O)COc1cc2cc(Nc3nc(N4CCC5(CCN(CC6CCN(c7ccc8c(C9CCC(=O)NC9=O)nn(C)c8c7)CC6)CC5)CC4)ncc3Cl)ccc2n(C)c1=O]

### Preparation of tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate

To a stirred solution of 3-(2,6-bis(benzyloxy)pyridin-3-yl)-6-bromo-1-methyl-1H-indazole (1.0 g, 2.0 mmol) and tert-butyl 9-(piperidin-4-ylmethyl)-3,9-diazaspiro[5.5]undecane-3-carboxylate (1.40 g, 4.0 mmol)(see, e.g., International Publication No. WO 2022/228547) in 1,4-dioxane (20 mL), cesium carbonate (1.95 g, 6.0 mmol) was added. The reaction was degassed with argon for 10 minutes. RuPhos (0.09 g, 0.20 mmol) and RuPhos-PdG3 (0.08 g, 0.10 mmol) were added, and the reaction was degassed for 5 minutes. The reaction mixture was stirred at 80° C. for 3 hours. The reaction mixture was cooled to room temperature and filtered through a celite bed. The filtrate was concentrated to isolate crude product. The crude product was purified by flash column chromatography (SiO₂, 230-400, 30% Ethyl acetate in petroleum ether) to afford tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate (0.7 g) as an off-white solid.

LC-MS (ESI): m/z=771.83 [M+H]⁺.

### Preparation of tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate

To a stirred solution of tert-butyl 9-((1-(3-(2,6-bis(benzyloxy)pyridin-3-yl)-1-methyl-1H-indazol-6-yl)piperidin-4-yl)methyl)-3,9-diazaspiro[5.5]undecane-3-carboxylate (0.7 g, 0.90 mmol) in THF (28 mL), 20% Pd(OH)₂ (0.7 g) was added at room temperature. The reaction was put under hydrogen pressure (60 psi) and stirred for 8 hours at room temperature. The reaction mixture was filtered through a celite bed. The celite was washed with ethyl acetate and concentrated under reduced pressure to isolate crude product. The crude product was washed with n-pentane and dried to afford tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate (0.60 g) as a brown solid. LC-MS (ESI): m/z=593.67 [M+H]⁺.

### Preparation of 3-(6-(4-((3,9-diazaspiro[5.5]undecan-3-yl)methyl)piperidin-1-yl)-1-methyl-1H-indazol-3-yl)piperidine-2,6-dione

A stirred solution of tert-butyl 9-((1-(3-(2,6-dioxopiperidin-3-yl)-1-methyl-1H-indazol-6-yl)piperidin-4-yl)methyl)-3,9-diazaspiro[5.5]undecane-3-carboxylate (0.6 g, 1.01 mmol) in DCM (12 mL) was cooled to 0° C. TFA (6 mL) was added and the reaction mixture was allowed to warm to room temperature over 3 hours with stirring. The reaction mixture was concentrated under reduced pressure to isolate the crude product. The crude product was triturated with diethyl ether (20 mL) to afford 3-(6-(4-((3,9-diazaspiro[5.5]undecan-3-yl)methyl)piperidin-1-yl)-1-methyl-1H-indazol-3-yl)piperidine-2,6-dione (0.44 g) as a brown solid.

LC-MS (ESI): m/z=493.37 [M+H]⁺.

### Preparation of 3-(6-(4-((9-(5-chloro-4-((1-methyl-2-oxo-3-(2-oxopropoxy)-1,2-dihydroquinolin-6-yl)amino)pyrimidin-2-yl)-3,9-diazaspiro[5.5]undecan-3-yl)methyl)piperidin-1-yl)-1-methyl-1H-indazol-3-yl)piperidine-2,6-dione

To a stirred suspension of 3-(6-(4-((3,9-diazaspiro[5.5]undecan-3-yl)methyl)piperidin-1-yl)-1-methyl-1H-indazol-3-yl)piperidine-2,6-dione (0.15 g, 0.31 mmol) in DMSO (3 mL), N, N-diisopropylethylamine (0.33 mL, 1.86 mol) and 6-((2,5-dichloropyrimidin-4-yl)amino)-1-methyl-3-(2-oxopropoxy) quinolin-2 (1H)-one (0.072 g, 0.18 mmol)(see, e.g., International Publication No. WO 2021/077010) were added. The reaction was heated at 110° C. for 16 hours. Reaction solvent was removed using a centrifugal evaporator to obtain crude product. The crude product was purified by prep-HPLC to afford 3-(6-(4-((9-(5-chloro-4-((1-methyl-2-oxo-3-(2-oxopropoxy)-1,2-dihydroquinolin-6-yl)amino)pyrimidin-2-yl)-3,9-diazaspiro[5.5]undecan-3-yl)methyl)piperidin-1-yl)-1-methyl-1H-indazol-3-yl)piperidine-2,6-dione (0.006 g) as an off-white solid.

LC-MS (ESI): m/z=849.51 [M+H]⁺.

¹H NMR (400 MHz, DMSO-d₆): δ 10.80 (s, 1H), 8.80 (s, 1H), 8.02 (s, 1H), 7.89 (d, J=2.00 Hz, 1H), 7.72 (dd, J=2.40, 9.00 Hz, 1H), 7.46 (dd, J=3.20, 9.00 Hz, 1H), 7.02 (s, 1H), 6.89 (d, J=9.20 Hz, 1H), 6.81 (s, 1H), 4.91 (s, 2H), 4.24-4.23 (m, 1H), 3.88 (s, 3H), 3.76 (d, J=12.40 Hz, 2H), 3.67 (s, 3H), 3.62 (s, 4H), 2.73 (d, J=11.20 Hz, 2H), 2.60-2.59 (m, 2H), 2.36 (s, 4H), 2.30 (s, 1H), 2.15-2.13 (m, 6H), 1.76-1.70 (m, 7H), 1.48 (s, 3H), 1.40 (s, 3H), 1.26 (s, 1H).

### Example 105. 2-((6-((5-chloro-2-((3R)-3-(((3-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide (Compound 275a)

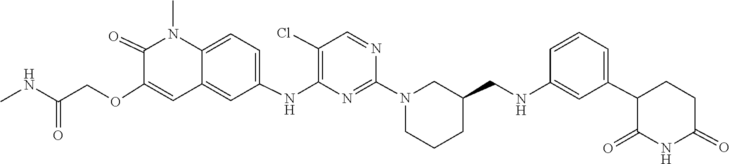 [SMILES: CNC(=O)COc1cc2cc(Nc3nc(N4CCC[C@H](CNc5cccc(C6CCC(=O)NC6=O)c5)C4)ncc3Cl)ccc2n(C)c1=O]

### Preparation of tert-butyl (R)-3-(((3-(2,6-bis(benzyloxy)pyridin-3-yl)phenyl)amino)methyl)piperidine-1-carboxylate

To a stirred solution of 2,6-bis(benzyloxy)-3-(3-bromophenyl)pyridine (1.50 g, 3.36 mmol), tert-butyl (R)-3-(aminomethyl)piperidine-1-carboxylate (1.08 g, 5.04 mmol) in 1,4-dioxane, was added potassium tert-butoxide (1.11 g, 10.08 mmol) and degassed with argon for 10 minutes. To this mixture was added bis(tri-tert-butylphosphine)palladium (0) (0.05 g, 0.10 mmol). The reaction mixture was stirred at 80° C. for 4 h. The reaction mixture was cooled to room temperature and filtered through a celite bed. The filtrate was concentrated to get crude product which was purified by flash column chromatography (8% ethyl acetate in petroleum ether) to afford tert-butyl (R)-3-(((3-(2,6-bis(benzyloxy)pyridin-3-yl)phenyl)amino)methyl)piperidine-1-carboxylate (1.20 g) as a pale yellow sticky compound.

LC-MS (ESI): m/z=580.68 [M+H]+

### Preparation of tert-butyl (3R)-3-(((3-(2,6-dioxopiperidin-3-yl)phenyl)amino)methyl)piperidine-1-carboxylate

To a stirred solution of tert-butyl (R)-3-(((3-(2,6-bis(benzyloxy)pyridin-3-yl)phenyl)amino)methyl)piperidine-1-carboxylate (1.20 g, 2.07 mmol) in THF was added acetic acid (1.2 mL) and degassed with nitrogen for 5 minutes. To this mixture was added palladium hydroxide on carbon, powder (2.40 g). The mixture was stirred under hydrogen atmosphere (15 psi) at room temperature for 4 h. The reaction mixture was filtered through celite and washed with THF:DCM (1:1). The filtrate was concentrated, dried to get a crude product and was washed with n-pentane and dried to afford tert-butyl (3R)-3-(((3-(2,6-dioxopiperidin-3-yl)phenyl)amino)methyl)piperidine-1-carboxylate (0.85 g) as a brown sticky compound which was used in the next step without further purification.

LC-MS (ESI): m/z=402.42 [M+H]+

### Preparation of 3-(3-((((S)-piperidin-3-yl)methyl)amino)phenyl)piperidine-2,6-dione

To a stirred solution of tert-butyl (3R)-3-(((3-(2,6-dioxopiperidin-3-yl)phenyl)amino)methyl)piperidine-1-carboxylate (0.85 g, 2.12 mmol) in DCM cooled to 0° C. was added TFA (5.1 mL). The mixture was stirred at room temperature for 2 h. The reaction mixture was concentrated to get the crude product which was triturated with petroleum ether (3 times), decanted, and dried to afford 3-(3-((((S)-piperidin-3-yl)methyl)amino)phenyl)piperidine-2,6-dione (0.80 g) as a brown liquid.

LC-MS (ESI): m/z=302.34 [M+H]+

### Preparation of 2-((6-((5-chloro-2-((3R)-3-(((3-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide

To a stirred solution of 3-(3-((((S)-piperidin-3-yl)methyl)amino)phenyl)piperidine-2,6-dione (0.35 g, 1.16 mmol) and 2-((6-((2,5-dichloropyrimidin-4-yl)amino)-1-methyl-2-oxo-1,2-dihydroquinolin-3-yl)oxy)-N-methylacetamide (0.19 g, 0.46 mmol) in DMSO was added N,N-diisopropylethylamine (1.61 mL, 9.30 mmol). The reaction mixture was stirred at 100° C. for 5 h. The reaction mixture was cooled to room temperature and poured into ice water and stirred for 15 minutes. The resulting precipitate was filtered and dried to get the crude product which was purified by prep-HPLC to afford 2-((6-((5-chloro-2-((3R)-3-(((3-(2,6-dioxopiperidin-3-yl)phenyl)amino)methyl)piperidin-1-yl)pyrimidin-4-yl)amino)-1-methyl-2-oxo-1,2-dihydroquinolin-3-yl)oxy)-N-methylacetamide as a pale brown solid (52 mg).

¹H NMR; 400 MHZ, DMSO-d₆: δ 10.80 (s, 1H), 8.82 (s, 1H), 8.03 (d, J=0.80 Hz, 1H), 7.93-7.80 (m, 3H), 7.43 (d, J=9.20 Hz, 1H), 7.13 (s, 1H), 6.96 (t, J=7.60 Hz, 1H), 6.37-6.33 (m, 3H), 5.62 (s, 1H), 4.53-4.46 (m, 3H), 4.30 (d, J=12.00 Hz, 1H), 3.63 (s, 4H), 2.97-2.89 (m, 3H), 2.75 (t, J=11.60 Hz, 1H), 2.65 (d, J=4.40 Hz, 3H), 2.59-2.56 (m, 1H), 2.49-2.44 (m, 1H), 2.07-1.67 (m, 5H), 1.41-1.18 (m, 2H).

LC-MS (ESI): m/z=673.43 [M+H]+

### Example 106:2-((6-((5-chloro-2-((2S)-2-(((3-(2,6-dioxopiperidin-3-yl)-1-methyl-1H-indazol-7-yl)amino)methyl)pyrrolidin-1-yl)pyrimidin-4-yl)amino)-1-methyl-2-oxo-1,2-dihydroquinolin-3-yl)oxy)-N-methylacetamide (Compound 300b)

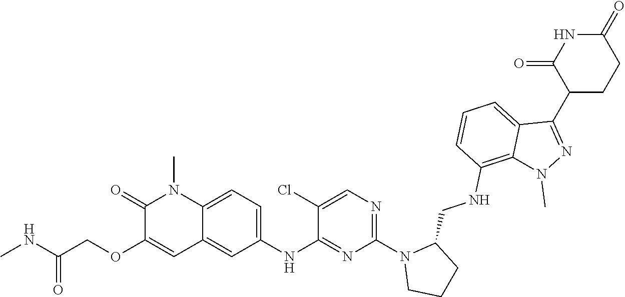 [SMILES: CNC(=O)COc1cc2cc(Nc3nc(N4CCC[C@H]4CNc4cccc5c(C6CCC(=O)NC6=O)nn(C)c45)ncc3Cl)ccc2n(C)c1=O]

### Preparation of tert-butyl(S)-2-(((3-(2,6-bis(benzyloxy)pyridin-3-yl)-1-methyl-1H-indazol-7-yl)amino)methyl)pyrrolidine-1-carboxylate

A solution of 3-(2,6-bis(benzyloxy)pyridin-3-yl)-7-bromo-1-methyl-1H-indazole (0.50 g, 0.1 mmol), tert-butyl(S)-2-(aminomethyl)pyrrolidine-1-carboxylate (0.40 g, 2.0 0 mmol) and potassium tertiary butoxide (0.33 g, 3.00 mmol) in 1,4-dioxane (10 mL) was degassed with argon for 5 minutes. To this mixture was added bis(tri-tert-butylphosphine)palladium (0) (0.051 g, 0.1 mmol). The reaction mixture was heated at 100° C. and stirred for 2 h. The reaction mixture was filtered through celite and concentrated. The crude was purified by flash column chromatography (neutral alumina; 1 to 2% methanol in dichloromethane) to afford tert-butyl(S)-2-(((3-(2,6-bis(benzyloxy)pyridin-3-yl)-1-methyl-1H-indazol-7-yl)amino)methyl)pyrrolidine-1-carboxylate (0.35 g) as an off white solid.

LC-MS (ESI): m/z=620.62 [M+H]⁺

### Preparation of tert-butyl (2S)-2-(((3-(2,6-dioxopiperidin-3-yl)-1-methyl-1H-indazol-7-yl)amino)methyl)pyrrolidine-1-carboxylate

A stirred solution of tert-butyl(S)-2-(((3-(2,6-bis(benzyloxy)pyridin-3-yl)-1-methyl-1H-indazol-7-yl)amino)methyl)pyrrolidine-1-carboxylate (0.35 g, 0.56 mmol) in THF (19.0 mL) was degassed with nitrogen. To this mixture was added palladium hydroxide on carbon (0.7 g). The mixture was stirred under H₂ (15 psi) at room temperature for 8 h. The reaction mixture was filtered through celite, washed with THE, and concentrated completely to obtain crude product which was washed with n-pentane and dried to afford tert-butyl (2S)-2-(((3-(2,6-dioxopiperidin-3-yl)-1-methyl-1H-indazol-7-yl)amino)methyl)pyrrolidine-1-carboxylate (0.25 g) as a brown solid.

LC-MS (ESI): m/z=442.03 [M+H]⁺

### Preparation of 3-(1-methyl-7-((((S)-pyrrolidin-2-yl)methyl)amino)-1H-indazol-3-yl)piperidine-2,6-dione

To a solution of tert-butyl (2S)-2-(((3-(2,6-dioxopiperidin-3-yl)-1-methyl-1H-indazol-7-yl)amino)methyl)pyrrolidine-1-carboxylate in DCM cooled to 0° C. was added TFA. The reaction mixture was stirred at room temperature for 1.5 h. The reaction mixture was concentrated completely to get crude product which was washed with n-pentane to afford 3-(1-methyl-7-((((S)-pyrrolidin-2-yl)methyl)amino)-1H-indazol-3-yl)piperidine-2,6-dione (0.16 g) as a brown gummy solid.

LC-MS (ESI): m/z=342.40 [M+H]⁺

### Preparation of 2-((6-((5-chloro-2-((2S)-2-(((3-(2,6-dioxopiperidin-3-yl)-1-methyl-1H-indazol-7-yl)amino)methyl)pyrrolidin-1-yl)pyrimidin-4-yl)amino)-1-methyl-2-oxo-1,2-dihydroquinolin-3-yl)oxy)-N-methylacetamide

To a stirred solution 3-(1-methyl-7-((((S)-pyrrolidin-2-yl)methyl)amino)-1H-indazol-3-yl)piperidine-2,6-dione (0.16 g, 0.46 mmol) and 2-((6-((2,5-dichloropyrimidin-4-yl)amino)-1-methyl-2-oxo-1,2-dihydroquinolin-3-yl)oxy)-N-methylacetamide (0.076 g, 0.18 mmol) in DMSO was added N,N-diisopropylethylamine (0.48 g, 3.72 mmol). The mixture was stirred at 80° C. for 6 h. The reaction mixture was poured into ice cold water and stirred for 15 minutes. The resulting precipitate was filtered and dried to get crude product which was purified by prep-HPLC to afford 2-((6-((5-chloro-2-((2S)-2-(((3-(2,6-dioxopiperidin-3-yl)-1-methyl-1H-indazol-7-yl)amino)methyl)pyrrolidin-1-yl)pyrimidin-4-yl)amino)-1-methyl-2-oxo-1,2-dihydroquinolin-3-yl)oxy)-N-methylacetamide (55 mg) as an off white solid.

¹H NMR (400 MHZ, DMSO-d₆): δ 10.85 (s, 1H), 8.82 (s, 1H), 8.35-8.09 (m, 2H), 7.94-7.73 (m, 3H), 6.15-6.23 (m, 4H), 4.56-4.23 (m, 7H), 3.67 (s, 2H), 3.55-3.47 (m, 3H), 3.14-3.03 (m, 2H), 2.66-2.59 (m, 5H), 2.30-2.26 (m, 1H), 2.17-1.95 (m, 5H), 1.24 (m, 1H).

LC-MS (ESI): m/z=713.46 [M+H]⁺

### Example 107:2-((6-((5-chloro-2-((((1s,4s)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide (Compound 289a)

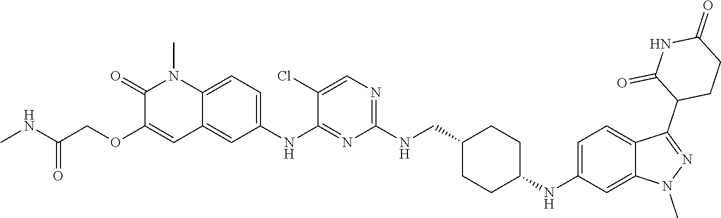 [SMILES: CNC(=O)COc1cc2cc(Nc3nc(NC[C@H]4CC[C@@H](Nc5ccc6c(C7CCC(=O)NC7=O)nn(C)c6c5)CC4)ncc3Cl)ccc2n(C)c1=O]

### Preparation of tert-butyl (((1s,4s)-4-((3-(2,6-bis(benzyloxy)pyridin-3-yl)-1-methyl-1H-indazol-6-yl)amino)cyclohexyl)methyl) carbamate

To a stirred solution of 3-(2,6-bis(benzyloxy)pyridin-3-yl)-6-bromo-1-methyl-1H-indazole (1.31 g, 2.62 mmol) in 1,4-dioxane (10.0 mL) was added tert-butyl (((1s,4s)-4-aminocyclohexyl)methyl) carbamate (0.50 g, 2.19 mmol), and purged with nitrogen for 15 minutes. To this mixture was added cesium carbonate (2.14 g, 6.56 mmol), Ruphos, (0.20 g, 0.43 mmol) and Ruphos pd G3 (0.09 g, 0.1 mmol). The mixture was heated at 120° C. and stirred for 4 h. The reaction was quenched with water and extracted in ethyl acetate. The organic layer was dried over anhydrous Na₂SO₄, filtered and concentrated to get crude product which was purified by flash column chromatography (25% ethyl acetate in petroleum ether) to afford tert-butyl (((1s,4s)-4-((3-(2,6-bis(benzyloxy)pyridin-3-yl)-1-methyl-1H-indazol-6-yl)amino)cyclohexyl)methyl) carbamate (0.55 g) as a yellow solid.

### Preparation of tert-butyl (((1s,4s)-4-((3-(2,6-dioxopiperidin-3-yl)-1-methyl-1H-indazol-6-yl)amino)cyclohexyl)methyl) carbamate

To a stirred solution of tert-butyl (((1s,4s)-4-((3-(2,6-bis(benzyloxy)pyridin-3-yl)-1-methyl-1H-indazol-6-yl)amino)cyclohexyl)methyl) carbamate (0.55 g, 0.84 mmol) in THF (16 mL) was added 20% Pd(OH)₂ on carbon, moisture 50% wet (1.1 g). The mixture was stirred under hydrogen atmosphere at room temperature for 5 h. The reaction mixture was diluted with ethyl acetate and filtered through celite bed and washed with 30% THF in ethyl acetate. The collected filtrate was concentrated under vacuum to get crude tert-butyl (((1s,4s)-4-((3-(2,6-dioxopiperidin-3-yl)-1-methyl-1H-indazol-6-yl)amino)cyclohexyl)methyl) carbamate (0.37 g) as an off white solid which was used in the next step without purification.

LC-MS (ESI): m/z=470.59 [M+H]⁺

### Preparation of 3-(6-(((1s,4s)-4-(aminomethyl)cyclohexyl)amino)-1-methyl-1H-indazol-3-yl)piperidine-2,6-dione

To a stirred solution of tert-butyl (((1s,4s)-4-((3-(2,6-dioxopiperidin-3-yl)-1-methyl-1H-indazol-6-yl)amino)cyclohexyl)methyl) carbamate (0.35 g, 0.74 mmol) in DCM (3.5 mL) was added HCl in dioxane (1.75 mL, 4 M). The mixture was stirred at room temperature for 16 h. The reaction mixture was concentrated under vacuum to get crude product which was triturated using diethyl ether to afford 3-(6-(((1s,4s)-4-(aminomethyl)cyclohexyl)amino)-1-methyl-1H-indazol-3-yl)piperidine-2,6-dione hydrochloride (0.26 g).

LC-MS (ESI): m/z=370.50 [M+H]⁺

### Preparation of 2-((6-((5-chloro-2-((((1s,4s)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide

To a stirred solution of 3-(6-(((1s,4s)-4-(aminomethyl)cyclohexyl)amino)-1-methyl-1H-indazol-3-yl)piperidine-2,6-dione hydrochloride (0.26 g, 0.64 mmol) in DMSO (7.8 mL) was added 2-((6-((2,5-dichloropyrimidin-4-yl)amino)-1-methyl-2-oxo-1,2-dihydroquinolin-3-yl)oxy)-N-methylacetamide (0.18 g, 0.45 mmol) and DIPEA (1.18 mL, 6.4 mmol). The mixture was heated to 120° C. for 4 h. The reaction mixture was quenched with ice-cold water, and the solid precipitate was filtered and dried under vacuum to get a crude product which was purified by prep HPLC to afford 2-((6-((5-chloro-2-((((1s,4s)-4-((3-(2,6-dioxopiperidin-3-yl)-1-methyl-1H-indazol-6-yl)amino)cyclohexyl)methyl)amino)pyrimidin-4-yl)amino)-1-methyl-2-oxo-1,2-dihydroquinolin-3-yl)oxy)-N-methylacetamide (0.037 g) as a light brown solid.

¹H NMR (400 MHZ, DMSO-d₆): δ 10.79 (s, 1H), 8.69 (bs, 1H), 8.43 (s, 1H), 8.0-7.83 (m, 3H), 7.45-7.43 (d, J=9.2 Hz, 1H), 7.31-6.96 (m, 3H), 6.57 (bs, 1H), 6.32 (s, 1H), 5.73 (m, 1H), 4.54 (s, 1H), 4.18-4.15 (m, 1H), 3.81 (s, 3H), 3.67 (s, 3H), 3.53-3.50 (m, 1H), 3.16 (m, 2H), 2.65-2.58 (m, 5H), 2.29-2.25 (m, 1H), 2.14-2.07 (m, 1H), 1.68-1.49 (m, 9H).

LC-MS (ESI): m/z=741.25 [M+H]⁺

### Example 108:2-((6-((5-chloro-2-((((1r,4r)-4-((4-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (Compound 277a)

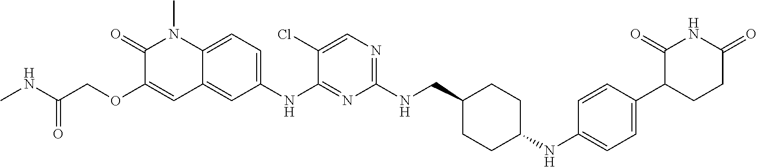 [SMILES: CNC(=O)COc1cc2cc(Nc3nc(NC[C@H]4CC[C@H](Nc5ccc(C6CCC(=O)NC6=O)cc5)CC4)ncc3Cl)ccc2n(C)c1=O]

### Preparation of tert-butyl (((1r,4r)-4-((4-(2,6-bis(benzyloxy)pyridin-3-yl)phenyl)amino)cyclohexyl)methyl) carbamate

A stirred solution of 2,6-bis(benzyloxy)-3-(4-bromophenyl)pyridine (0.75 g, 1.68 mmol) and tert-butyl (((1r,4r)-4-aminocyclohexyl)methyl) carbamate (0.42 g, 1.84 mmol) in 1,4-dioxane (15 mL) was purged with N₂. To this mixture was added potassium tert-butoxide (0.56 g, 5.04 mmol) and brettPhos-Pd-G3 (0.08 g, 0.08 mmol). The resultant reaction mixture was heated to 80° C. and stirred for 3 h. The reaction mixture was quenched with water and extracted with ethyl acetate. The combined organic layers were dried over anhydrous sodium sulphate, filtered and concentrated. The crude product was purified by flash column chromatography (20% ethyl acetate in petroleum ether) to afford tert-butyl (((1r,4r)-4-((4-(2,6-bis(benzyloxy)pyridin-3-yl)phenyl)amino)cyclohexyl)methyl) carbamate (0.70 g) as a yellow sticky substance.

LC-MS (ESI): m/z=594.61 [M+H]⁺

### Preparation of tert-butyl (((1r,4r)-4-((4-(2,6-dioxopiperidin-3-yl)phenyl)amino)cyclohexyl)methyl) carbamate

A stirred solution of tert-butyl (((1r,4r)-4-((4-(2,6-bis(benzyloxy)pyridin-3-yl)phenyl)amino)cyclohexyl)methyl) carbamate (0.70 g, 1.18 mmol) in tetrahydrofuran (15 mL) was purged with N₂ at room temperature. To this mixture was added palladium hydroxide on carbon, 20% Pd (0.70 g, w/w). The reaction was stirred under hydrogen atmosphere with balloon pressure at room temperature for 16 h. After completion, the reaction mixture was filtered through celite and washed with DCM. The combined organic layers were concentered to get crude product which was purified by flash column chromatography (50% ethyl acetate in petroleum ether) to afford tert-butyl (((1r,4r)-4-((4-(2,6-dioxopiperidin-3-yl)phenyl)amino)cyclohexyl)methyl) carbamate (0.45 g) as a colorless liquid.

LC-MS (ESI): m/z=416.52 [M+H]⁺

### Preparation of 3-(4-(((1r,4r)-4-(aminomethyl)cyclohexyl)amino)phenyl)piperidine-2,6-dione

To a stirred solution of tert-butyl (((1r,4r)-4-((4-(2,6-dioxopiperidin-3-yl)phenyl)amino)cyclohexyl)methyl) carbamate (0.45 g, 0.60 mmol) in DCM (9.0 mL) was added 4M HCl in 1,4-dioxane (1.35 mL) at 0° C. under N₂ atmosphere. The resulting reaction mixture was stirred at room temperature for 5 h. The reaction mixture was concentrated under reduced pressure and co-distilled with DCM. The crude product was purified by triturating with diethyl ether followed by n-pentane to afford 3-(4-(((1r,4r)-4-(aminomethyl)cyclohexyl)amino)phenyl)piperidine-2,6-dione as a white solid (0.34 g).

### Preparation of 2-((6-((5-chloro-2-((((1r,4r)-4-((4-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide

A stirred solution of 2-((6-((2,5-dichloropyrimidin-4-yl)amino)-1-methyl-2-oxo-1,2-dihydroquinolin-3-yl)oxy)-N-methylacetamide hydrochloride (0.30 g, 0.95 mmol) in DMSO (9.0 mL) was purged with N₂. To this mixture was added DIPEA (0.87 mL, 4.75 mmol). The resulting mixture was stirred for 10 min followed by the addition of 3-(4-(((1r,4r)-4-(aminomethyl)cyclohexyl)amino)phenyl)piperidine-2,6-dione (0.15 g, 0.38 mmol) and pTSA (Cat.)(10 mg). The resulting reaction mixture was heated to 160° C. for 3 h. The reaction mixture was quenched with cold water, filtered and washed with cold water followed by petroleum ether. The crude product was purified by prep-HPLC to afford 2-((6-((5-chloro-2-((((1r,4r)-4-((4-(2,6-dioxopiperidin-3-yl)phenyl)amino)cyclohexyl)methyl)amino)pyrimidin-4-yl)amino)-1-methyl-2-oxo-1,2-dihydroquinolin-3-yl)oxy)-N-methylacetamide (0.034 g) as an off-white solid.

¹H NMR (400 MHZ, DMSO-d₆): δ 10.76 (s, 1H), 8.69 (bs, 1H), 8.01-7.82 (m, 4H), 7.45-7.42 (d, J=12.0 Hz, 1H), 7.18-6.85 (m, 4H), 6.47 (d, J=8.0 Hz, 2H), 5.25 (bs, 1H), 4.56 (s, 2H), 3.67 (s, 3H), 3.60 (m, 1H), 3.07-3.05 (m, 3H), 2.66-2.65 (d, J=4.4 Hz, 4H), 2.42-2.40 (m, 1H), 2.05-1.94 (m, 4H), 1.74 (m, 2H), 1.52 (m, 1H), 1.01 (m, 4H).

LC-MS (ESI): m/z=685.49 [M−H]⁻

### Example 109: 2-((6-((5-chloro-2-((3R)-3-((3-(2,6-dioxopiperidin-3-yl)-1-methyl-1H-indazol-6-yl)amino)piperidin-1-yl)pyrimidin-4-yl)amino)-1-methyl-2-oxo-1,2-dihydroquinolin-3-yl)oxy)-N-methylacetamide (Compound 280b)

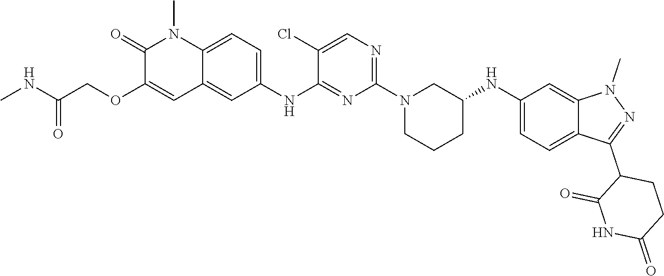 [SMILES: CNC(=O)COc1cc2cc(Nc3nc(N4CCC[C@@H](Nc5ccc6c(C7CCC(=O)NC7=O)nn(C)c6c5)C4)ncc3Cl)ccc2n(C)c1=O]

### Preparation tert-butyl (R)-3-((3-(2,6-bis(benzyloxy)pyridin-3-yl)-1-methyl-1H-indazol-6-yl)amino)piperidine-1-carboxylate

A solution of 3-(2,6-bis(benzyloxy)pyridin-3-yl)-6-bromo-1-methyl-1H-indazole (0.7 g, 1.4 mmol), tert-butyl (R)-3-aminopiperidine-1 carboxylate (0.33 g, 1.68 mmol) and sodium tertiary butoxide (0.40 g, 4.2 mmol) in 1,4-dioxane (14 mL) was degassed with argon for 5 minutes. To this mixture was added BrettPhos Pd G3 (0.12 g, 0.14 mmol). The reaction mixture was heated at 100° C. for 4 h. The reaction mixture was filtered through celite bed and concentrated completely. The crude was purified by column chromatography (2% methanol in dichloromethane) to afford tert-butyl (R)-3-((3-(2,6-bis(benzyloxy)pyridin-3-yl)-1-methyl-1H-indazol-6-yl)amino)piperidine-1-carboxylate (0.70 g) as a brown liquid.

LC-MS (ESI): m/z=621.14 [M+H]+

### Preparation of tert-butyl (3R)-3-((3-(2,6-dioxopiperidin-3-yl)-1-methyl-1H-indazol-6-yl)amino)piperidine-1-carboxylate

To a stirred solution of tert-butyl (R)-3-((3-(2,6-bis(benzyloxy)pyridin-3-yl)-1-methyl-1H-indazol-6-yl)amino)piperidine-1-carboxylate (0.70 g, 1.13 mmol) in THF (20 mL), acetic acid (0.7 mL), DMF (4.2 mL), dioxane (0.7 mL) was added, 20% Pd(OH)₂ on carbon (2.8 g). The mixture was stirred under hydrogen atmosphere (15 psi) at room temperature for 4 h. The reaction mixture was filtered through celite and concentrated to obtain crude product which was washed with n-pentane and dried to afford tert-butyl (3R)-3-((3-(2,6-dioxopiperidin-3-yl)-1-methyl-1H-indazol-6-yl)amino)piperidine-1-carboxylate (0.40 g) as a brown solid which was used directly in the next step without further purification.

LC-MS (ESI): m/z=442.48 [M+H]+

### Preparation of 3-(1-methyl-6-(((R)-piperidin-3-yl)amino)-1H-indazol-3-yl)piperidine-2,6-dione

To a stirred solution of tert-butyl (3R)-3-((3-(2,6-dioxopiperidin-3-yl)-1-methyl-1H-indazol-6-yl)amino)piperidine-1-carboxylate (0.40 g, 0.90 mmol)) in DCM (8 mL) cooled to 0° C. was added TFA (4 mL). The reaction mixture was stirred at room temperature for 1.5 h. The reaction mixture was concentrated to obtain crude product which was washed with n-pentane to afford 3-(1-methyl-6-(((R)-piperidin-3-yl)amino)-1H-indazol-3-yl)piperidine-2,6-dione (0.22 g) as a brown gummy solid.

LC-MS (ESI): m/z=342.40 [M+H]+

### Preparation of 2-((6-((5-chloro-2-((3R)-3-((3-(2,6-dioxopiperidin-3-yl)-1-methyl-1H-indazol-6-yl)amino)piperidin-1-yl)pyrimidin-4-yl)amino)-1-methyl-2-oxo-1,2-dihydroquinolin-3-yl)oxy)-N-methylacetamide

To a stirred solution of 3-(1-methyl-6-(((R)-piperidin-3-yl)amino)-1H-indazol-3-yl)piperidine-2,6-dione (0.22 g, 0.64 mmol) and 2-((6-((2,5-dichloropyrimidin-4-yl)amino)-1-methyl-2-oxo-1,2-dihydroquinolin-3-yl)oxy)-N-methylacetamide (0.10 g, 0.25 mmol) in DMSO was added N,N-Diisopropylethylamine (0.66 g, 4.4 mmol). The reaction mixture was stirred at 100° C. for 6 h. The reaction mixture was poured into ice cold water and stirred for 15 minutes. The resulting precipitate was filtered and dried to get crude product which was purified by prep-HPLC to afford 2-((6-((5-chloro-2-((3R)-3-((3-(2,6-dioxopiperidin-3-yl)-1-methyl-1H-indazol-6-yl)amino)piperidin-1-yl)pyrimidin-4-yl)amino)-1-methyl-2-oxo-1,2-dihydroquinolin-3-yl)oxy)-N-methylacetamide (0.31 g) as a brown solid.

¹H NMR (400 MHZ, DMSO-d₆): δ 10.80 (d, J=12.0 Hz, 1H), 8.86 (s, 1H), 8.08 (s, 1H), 7.93-7.78 (m, 1H), 7.31 (t, J=6.00 Hz, 1H), 6.95-5.93 (m, 3H), 4.84-4.75 (m, 1H), 4.70-4.05 (m, 4H), 3.99-3.33 (m, 7H), 3.22-3.01 (br, 2H) 2.93 (br s, 3H), 2.72-2.58 (m, 6H), 2.27-2.06 (m, 3H), 1.77 (br s, 1H), 1.56 (br s, 2H).

LC-MS (ESI): m/z=713.46 [M+H]+

### Example B1

### Hibit Based Degradation Assay

Human BCL6 protein coding open reading frame fused with N-terminal HiBiT coding sequence was synthesized from Integrated DNA Technologies (IDT). Next, the N-HiBiT-BCL6 sequence was cloned into pLV-UBC-PGK-Puro, a lentivirus plasmid purchased from Vectorbuilder, to generate pLV-UBC-N-HiBiT-BCL6-PGK-puro. Lentiviral particles were generated from Lenti-X™ 293T cells (Clontech) by co-transfection of pLV-UBC-N-HiBiT-BCL6-PGK-puro plasmids and lentiviral packaging plasmid mix (Cellecta). HT1080 [HT1080] (ATCC CCL-121) cells were infected with the lentivirus, and HT1080 cells stably integrated with the lentiviral vectors were established by incubation with 1 μg/mL puromycin (Thermofisher).

HT1080 cells expressing N-terminal HiBiT tagged BCL6 were dispensed into a 384-well plate pre-spotted with compounds at varying concentrations. Five thousand cells were seeded into each well in 40 μL of RPMI 1640 media plus 10% Fetal Bovine Serum (10082-147, Thermofisher). After 5 hours of incubation at 37° C. with 5% CO₂, 30 μL of the NANO-GLO® HiBiT Lytic Detection System working solution (Promega) was added to each well and incubated at room temperature for 15 min. After incubation, luminescence was read on a PHERAstar FSX Plate Reader (BMG). BCL6 degradation at each indicated concentration was normalized with DMSO control. The BCL6 degradation curves were plotted using a four-parameter logistic model.

Table B1 provides the Yₘᵢₙ and EC₅₀ values for certain compounds of this disclosure.

TABLE B1

| Compound No. | Yₘᵢₙ (5 hr) | EC₅₀ (5 hr) |
|---|---|---|
| 101 | ++++ | ++++ |
| 102 | +++ | ++++ |
| 103 | ++++ | ++++ |
| 104 | +++ | +++ |
| 105 | ++ | ++ |
| 106a | ++ | +++ |
| 106b | ++ | +++ |
| 107 | ++++ | +++ |
| 108 | +++ | ++++ |
| 260a | ++ | ++ |
| 260b | + | + |
| 261a | ++ | ++++ |
| 261b | +++ | ++++ |
| 262a | ++ | + |
| 262b | ++ | ++ |
| 263 | ++ | +++ |
| 265 | ++ | +++ |
| 266a | ++ | +++ |
| 266b | ++ | +++ |
| 275a | + | + |
| 275b | ++ | +++ |
| 277a | + | +++ |
| 277b | ++ | +++ |
| 279a | ++ | ++ |
| 279b | +++ | +++ |
| 280a | ++ | ++ |
| 280b | + | + |
| 281 | +++ | +++ |
| 282 | +++ | ++++ |
| 283 | +++ | ++++ |
| 284 | ++ | +++ |
| 286 | +++ | ++++ |
| 287 | +++ | ++++ |
| 288 | +++ | ++++ |
| 289a | + | + |
| 289b | ++++ | ++++ |
| 290a | ++ | +++ |
| 290b | +++ | +++ |
| 292 | +++ | ++++ |
| 293a | +++ | +++ |
| 293b | ++ | ++++ |
| 294a | ++ | +++ |
| 294b | ++ | +++ |
| 295a | ++ | +++ |
| 295b | ++ | ++ |
| 296 | +++ | ++++ |
| 297a | ++ | ++++ |
| 297b | ++ | ++++ |
| 298 | +++ | ++++ |
| 299a | +++ | +++ |
| 300a | ++ | +++ |
| 300b | + | + |
| 301a | ++ | +++ |
| 301b | ++ | +++ |
| 302a | ++ | ++++ |
| 302b | ++ | ++++ |
| 303a | ++ | +++ |
| 303b | ++ | +++ |
| 304a | ++ | +++ |
| 304b | ++ | +++ |
| 305a | +++ | ++++ |
| 305b | +++ | ++++ |
| 306a | +++ | ++++ |
| 306b | ++++ | ++++ |
| 307a | ++++ | ++++ |
| 307b | +++ | ++++ |
| 308a | ++++ | +++ |
| 308b | ++++ | ++++ |
| 309a | +++ | ++++ |
| 309b | +++ | ++++ |
| 310 | +++ | ++++ |
| 311 | ++ | +++ |
| 312a | ++ | ++++ |
| 313a | ++ | ++++ |
| 314 | ++ | +++ |
| 315 | ++ | +++ |
| 316 | ++++ | ++++ |
| 317a | +++ | +++ |
| 318 | ++ | +++ |
| 319 | ++ | ++++ |
| 320 | +++ | +++ |
| 321 | +++ | ++++ |
| 322 | +++ | ++++ |
| 323 | +++ | ++++ |
| 324 | +++ | ++++ |
| 325 | +++ | ++++ |
| 326 | +++ | +++ |
| 327 | ++++ | ++++ |
| 328 | ++++ | ++++ |
| 329 | ++ | +++ |
| 330 | ++ | +++ |
| 331 | +++ | ++ |
| 332 | +++ | +++ |
| 333 | ++++ | ++++ |
| 334 | ++++ | ++++ |
| 335 | ++ | +++ |
| 336a | ++++ | ++++ |
| 337 | ++++ | +++ |
| 338 | ++ | ++++ |
| 339 | ++ | ++ |
| 340 | +++ | ++++ |
| 341 | ++++ | ++++ |
| 342 | +++ | ++++ |
| 343 | +++ | ++++ |
| 344 | ++ | ++++ |
| 345 | ++++ | ++++ |
| 346 | ++++ | ++++ |
| 347a | ++++ | ++++ |
| 348 | ++++ | ++++ |

Notes:
Yₘᵢₙ ≤ 25%: “++++”;
25% < Yₘᵢₙ ≤ 50%: “+++”;
50% < Yₘᵢₙ ≤ 70%: “++”;
Yₘᵢₙ > 70%: “+”
EC₅₀ ≤ 10 nM: “++++”;
10 nM < EC₅₀ ≤ 100 nM: “+++”;
100 nM < EC₅₀ ≤ 1000 nM: “++”;
EC₅₀ > 1000 nM: “+”.

### Example B2

### Cell Proliferation Assay

OCI-Ly1 (DSMZ: ACC 722), SU-DHL-5 (ATCC CRL-2958), Toledo (ATCC CRL-2631), and WSU-DLCL2 (DSMZ: ACC 575) cells are seeded at 1,000 cells per well in 50 μL RPMI 1640 media plus 10% Fetal Bovine Serum (10082-147, Thermofisher) in black 384-well plates. The plates are pre-spotted with compounds at varying concentrations. After 5 days of incubation at 37° C. with 5% CO₂, cell viabilities are assessed using CELLTITER-GLO® Cell Viability Assay kit according to manufacturer's instructions (Promega). Relative cell proliferation at each concentration is normalized against DMSO control.

### REFERENCES

### Oci-Ly1 Cells:

1) Chang, H., Blondal, et al. (1995). Leukemia & Lymphoma 19 (1-2): 165-171.
2) Tweeddale, M. E., et al. (1987). Blood 69 (5): 1307-1314.
3) Farrugia, M. M., et al. (1994). Blood 83 (1): 191-198.
4) Mehra, S., et al. (2002). Genes Chromosomes Cancer 33 (3): 225-234.
5) Küppers, R., et al. (2003). Journal of Clinical Investigation 111 (4): 529-537.
6) Ngo, V. N., et al. (2011). Nature 470 (7332): 115-9.

### WSU-DLCL2 Cells:

1) Al-Katib, A. M., et al. (1998). Clinical Cancer Research 4 (5): 1305-1314.
2) Mohammad, R. M., et al. (2000). Clinical Cancer Research 6 (12): 4950-4956.
3) Morin, R. D., et al. (2010). Nature Genetics 42 (2): 181-185.
4) Quentmeier, H., et al. (2019). Scientific Reports 9 (1): 8218.

### Example B3

### Pharmacokinetic Assessment

Pharmacokinetics (PK) studies were conducted on male CD-1 mice by two delivery routes: intravenous (IV) injection and oral gavage (PO). Mice for the IV group (n=3) were allowed free access to food and water and mice for the PO group (n=3) were fasted 6-8 hours prior to dosing. The test articles were formulated in solution for the IV route (commonly 5% DMSO/10% Solutol HS15/85% water (pH 5)) and solution or suspension for the PO route (commonly 100% PEG400). On the day of the experiment, test article was administered via vein injection (commonly 1 mg/kg) for IV route or via oral gavage (commonly 10 mg/kg) for PO route, respectively. Blood samples were collected via the saphenous vein using a needle (commonly 20G) at 0.83 to 24 hours post dose. Approximately 30 μL of blood per timepoint was collected into pre-chilled tubes using K₂EDTA as the anti-coagulant. After collection of blood samples at each time point, the blood samples were stored on ice. Blood samples were centrifuged within 0.5 hour of collection to separate plasma. Centrifugation was conducted at 2500×g for 15 minutes at 4° C. Plasma samples were immediately acidified (commonly 0.1M sodium citrate (pH=5) buffer). The samples were then submitted to LC-MS/MS for sample analysis. Pharmacokinetics parameters, including clearance (IV), area under the curve (AUC), and oral bioavailability (% F) were calculated by non-compartmental model.

Table B3 provides the C₁, Cₘₐₓ, AUC_{0-inf}, and % F values for certain compounds of this disclosure.

TABLE B3

| | Compound 101 | Compound 102 |
|---|---|---|
| Cl [mL/min/kg] | 12.85 | 10.80 |
| Cₘₐₓ (nM) | 3399.13 | 2551.26 |
| AUC_{0-inf} (hr*nM) | 12600.98 | 13459.43 |
| % F | 76.37 | 67.45 |

### Exemplary Embodiments

### P01 Embodiments

1. A compound of Formula (I):

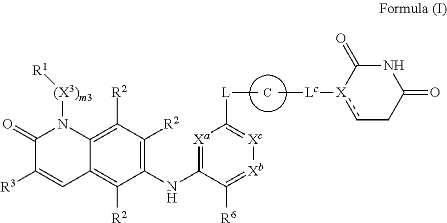

or a pharmaceutically acceptable salt thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
R³ is selected from the group consisting of:
-A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂(C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1},
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1}, L^{A3}, and L^{A4} provided that 1-3 occurrences of L^{A} is L^{A4}.

### (BB)

n1 is an integer from 0 to 20; and
each L^{A} is independently selected from the group consisting of: L^{A1} and L^{A3},
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{c};
Ring C is selected from the group consisting of:

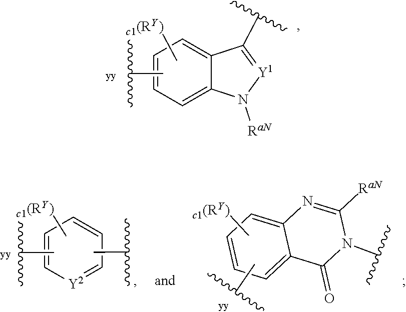

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂(C₁₋₆ alkyl);
(m) S(O)₀₋₂(C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(—O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.
2. The compound of Embodiment 1, wherein R³ is -A¹-C(R⁴R⁴)-A².
3. The compound of Embodiments 1 or 2, wherein A¹ is —O—.
4. The compound of any one of Embodiments 1-3, wherein each R⁴ is H.
5. The compound of any one of Embodiments 1-4, wherein A² is —C(O)NH₂ or —C(O)NR^{3A}R^{f}.
6. The compound of Embodiment 5, wherein R^{3A} is C₁₋₃ alkyl optionally substituted with 1-6 R^{c}.
7. The compound of Embodiments 1-6, wherein A² is —C(O)NH₂, —C(O)NHMe, or —C(O)NMe₂.
8. The compound of Embodiment 1, wherein R³ is

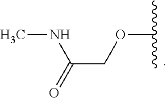 [SMILES: *OCC(=O)NC]

9. The compound of any one of Embodiments 1-8, wherein X^{a} is N; X^{c} is N; and X^{b} is CR^{x1} (e.g., CH).
10. The compound of any one of Embodiments 1-8, wherein X^{a} is CH; X^{c} is N; and X^{b} is CR^{x1} (e.g., CH).
11. The compound of any one of Embodiments 1-10, wherein R⁶ is —Cl or —F.
12. The compound of any one of Embodiments 1-11, wherein each R² is H.
13. The compound of any one of Embodiments 1-11, wherein one R² is selected from the group consisting of: halo, cyano, C₁₋₃ alkyl, C₁₋₃ haloalkyl, C₁₋₃ alkoxy, and C₁₋₃ haloalkoxy; and each remaining R² is H.
14. The compound of any one of Embodiments 1-13, wherein m3 is 0.
15. The compound of any one of Embodiments 1-13, wherein m3 is 1; and X³ is C₁₋₃ alkylene.
16. The compound of any one of Embodiments 1-15, wherein R¹ is H.
17. The compound of any one of Embodiments 1-13, wherein m3 is 1; X³ is methylene, ethylene, or isopropylene; and R¹ is H.
18. The compound of Embodiment 1, wherein:
R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{c};
each R² is H;
X^{a} is N or CH;
X^{c} is N;
X^{b} is CH; and
R⁶ is —F or —Cl.
19. The compound of Embodiment 18, wherein m3 is 1; X³ is methylene, ethylene, or isopropylene; and R¹ is H.
20. The compound of Embodiments 18 or 19, wherein X^{a} is N.
21. The compound of any one of Embodiments 18-20, wherein A² is C(O)NHMe.
22. The compound of any one of Embodiments 1-21, wherein Ring C is

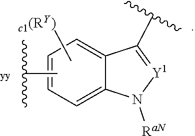

23. The compound of Embodiments 1-22, wherein Ring C is

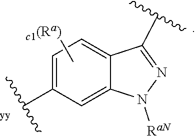

24. The compound of Embodiments 1-22, wherein Ring C is

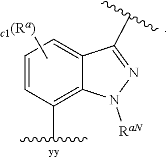

25. The compound of any one of Embodiments 1-21, wherein Ring C is

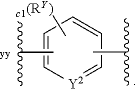

26. The compound of any one of Embodiments 1-25, wherein c1 is 0.
27. The compound of any one of Embodiments 1-25, wherein c1 is 1; and R^{Y} is halo (e.g., —F).
28. The compound of anyone of Embodiments 1-27, wherein X is CH.
29. The compound of any one of Embodiments 1-24 or 27-28, wherein R^{aN} is C₁₋₃ alkyl (e.g., methyl).
30. The compound of any one of Embodiments 1-29, wherein L^{C} is a bond.
31. The compound of any one of Embodiments 1-21, wherein the

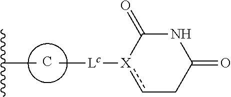

moiety is selected from the group consisting of:

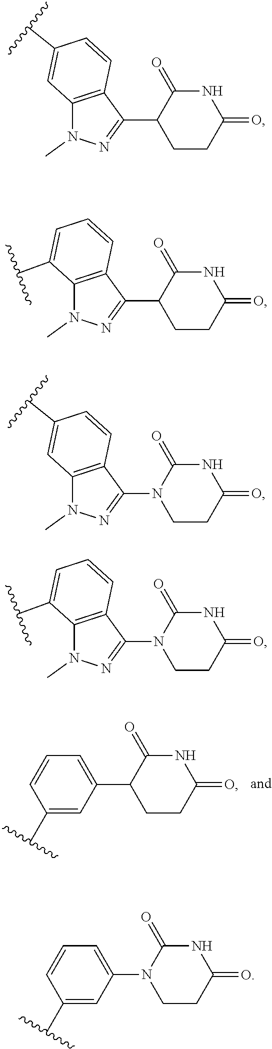

32. The compound of any one of Embodiments 1-21 or 31, wherein the

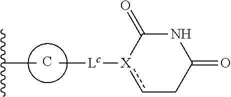

moiety is selected from the group consisting of:

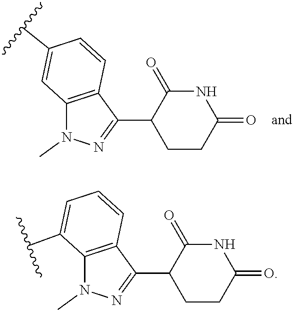

33. The compound of Embodiment 1, wherein the

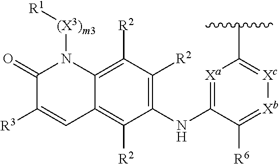

moiety is

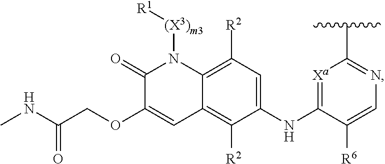

wherein:
m3 is 1;
X³ is C₁₋₃ alkylene;
R¹ is H;
X^{a} is CH or N;
X^{c} is N;
R⁶ is —F or —Cl; and
the

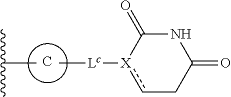

moiety is selected from the group consisting of:

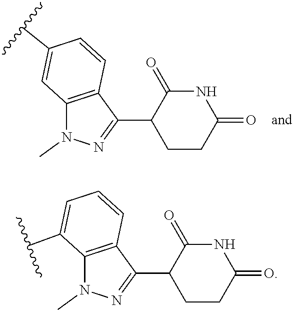

34. The compound of Embodiment 33, wherein —(X³)ₘ₃—R¹ is methyl, ethyl, or isopropyl.
35. The compound of Embodiments 33 or 34, wherein X^{a} is CH.
36. The compound of Embodiments 33 or 34, wherein X^{a} is N.
37. The compound of any one of Embodiments 1-36, wherein L is -(L^{A})ₙ₁-, wherein L^{A} and n1 are defined according to (AA).
38. The compound of any one of Embodiments 1-37, wherein n1 is an integer from 1 to 5.
39. The compound of any one of Embodiments 1-38, wherein n1 is an integer from 2 to 4 (e.g., 2 or 3).
40. The compound of any one of Embodiments 1-39, wherein L is selected from the group consisting of:
-L^{A4}-L^{A1}-L^{A4}-_{bb};
-L^{A4}-L^{A4}-_{bb};
-L^{A4}-L^{A3}-L^{A4}-_{bb}; and
-L^{A4}-L^{A1}-L^{A4}-L^{A3}-_{bb},
wherein bb represents the point of attachment to Ring C.
41. The compound of Embodiment 40, wherein each L^{A4} is independently a C₃₋₁₀ cycloalkylene or 4-12 membered heterocyclylene, each of which is optionally substituted with 1-6 R^{a}.
42. The compound of Embodiments 40 or 41, wherein each L^{A4} is independently a 4-12 (e.g., 4-10) membered nitrogen-containing heterocyclylene optionally substituted with 1-3 R^{a}.
43. The compound of any one of Embodiments 40-42, wherein each L^{A4} is independently a monocyclic 4-6 membered nitrogen-containing heterocyclylene optionally substituted with 1-3 R^{a}.
44. The compound of any one of Embodiments 40-42, wherein one L^{A4} is a monocyclic 4-6 membered nitrogen-containing heterocyclylene optionally substituted with 1-3 R^{a}; and the other L^{A4} is a bicyclic 6-12 (e.g., 6-10) membered nitrogen-containing heterocyclylene optionally substituted with 1-3 R^{a}.
45. The compound of any one of Embodiments 40-42 or 44, wherein one L^{A4} is a monocyclic 4-6 membered nitrogen-containing heterocyclylene optionally substituted with 1-3 R^{a}; and the other L^{A4} is a bicyclic spirocyclic 6-12 (e.g., 6-10) membered nitrogen-containing heterocyclylene optionally substituted with 1-3 R^{a}.
46. The compound of any one of Embodiments 40-42 or 44, wherein one L^{A4} is a monocyclic 4-6 membered nitrogen-containing heterocyclylene optionally substituted with 1-3 R^{a}; and the other L^{A4} is a bicyclic bridged 6-12 (e.g., 6-10) membered nitrogen-containing heterocyclylene optionally substituted with 1-3 R^{a}.
47. The compound of any one of Embodiments 42-46, wherein each L^{A4} contains 1-2 ring nitrogen atoms and no additional ring heteroatoms.
48. The compound of any one of Embodiments 41-47, wherein each R^{a} present on L^{A4} is independently selected from the group consisting of: —F, CN, C₁₋₃ alkoxy, OH, and C₁₋₃ alkyl optionally substituted with 1-3 F.
49. The compound of any one of Embodiments 40-48, wherein L is -L^{A4}-L^{A1}-L^{A4}-bb.
50. The compound of Embodiment 49, wherein L^{A1} is —CH₂—, —CHMe-, or —CMe₂-.
51. The compound of any one of Embodiments 40-48, wherein L is -L^{A4}-L^{A3}-L^{A4} bb.
52. The compound of Embodiment 51, wherein L^{A3} is —C(═O) or —O—.
53. The compound of any one of Embodiments 40-48, wherein L is -L^{A4}-L^{A1}-L^{A4}-L^{A3}-_{bb}, and L^{A3} is C(═O).
54. The compound of any one of Embodiments 1-39, wherein L is selected from the group consisting of:
-L^{A4}-L^{A3}-_{bb};
-L^{A4}-L^{A1}-_{bb}; and
-L^{A4}-L^{A1}-L^{A3}-_{bb},
wherein bb represents the point of attachment to Ring C.
55. The compound of Embodiment 54, wherein L is -L^{A4}-L^{A3}-_{bb}.
56. The compound of Embodiment 55, wherein L^{A3} is —NH— or —N(C₁₋₃ alkyl)- (e.g., —NH—).
57. The compound of any one of Embodiments 54-56, wherein L^{A4} is a 4-12 membered heterocyclylene optionally substituted with 1-6 R^{a}.
58. The compound of any one of Embodiments 54-57, wherein L^{A4} is a 4-12 (e.g., 6-10) membered nitrogen-containing heterocyclylene optionally substituted with 1-3 R^{a}.
59. The compound of any one of Embodiments 54-58, wherein L^{A4} is a bicyclic spirocyclic 6-12 (e.g., 6-10) membered nitrogen-containing heterocyclylene optionally substituted with 1-3 R^{a}.
60. The compound of any one of Embodiments 57-59, wherein L^{A4} contains 1-2 ring nitrogen atoms and no additional ring heteroatoms.
61. The compound of any one of Embodiments 57-60, wherein each R^{a} present on L^{A4} is independently selected from the group consisting of: —F, CN, C₁₋₃ alkoxy, OH, and C₁₋₃ alkyl optionally substituted with 1-3 F.
62. The compound of any one of Embodiments 1-40, wherein L is selected from the group consisting of the moieties delineated in Table L1-a:

TABLE L1-a

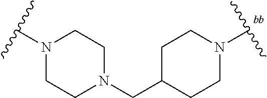

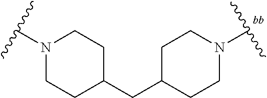

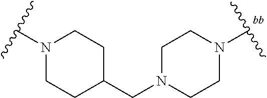

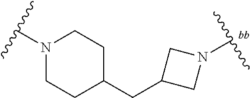

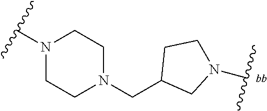

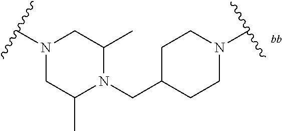

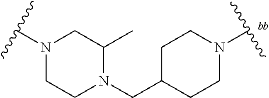

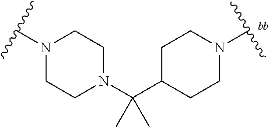

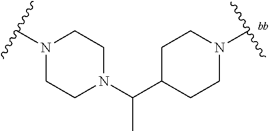

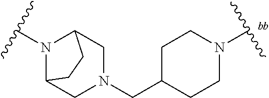

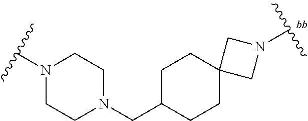 [SMILES: *N1CCN(CC2CCC3(CC2)CN(*)C3)CC1]

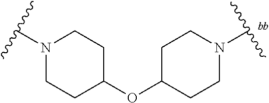

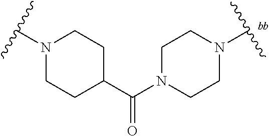

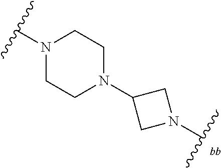

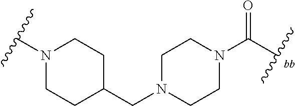

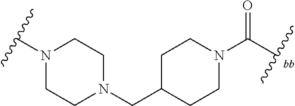

wherein ^{bb} represents the point of attachment to Ring C.

63. The compound of any one of Embodiments 1-39 or 54, wherein L is selected from the group consisting of the moieties delineated in Table L2-a:

TABLE L2-a

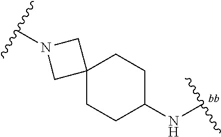

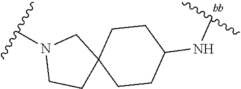

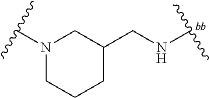

wherein ^{bb} represents the point of attachment to Ring C.

64. The compound of Embodiment 1, wherein the compound is a compound of Formula (I-a):

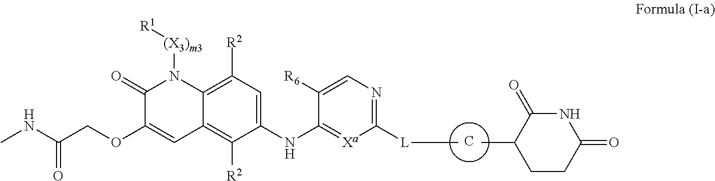

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

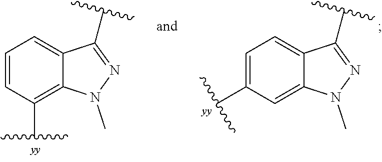 [SMILES: *c1nn(C)c2c(*)cccc12 ; *c1ccc2c(*)nn(C)c2c1]

L is selected from the group consisting of:
-L^{A4}-L^{A1}-L^{A4}-_{bb};
-L^{A4}-O-L^{A4}-_{bb};
-L^{A4}-C(═O)-L^{A4}-_{bb}; and
-L^{A}-L^{A1}-L^{A4}-C(═O)-_{bb},
wherein bb represents the point of attachment to Ring C;
L^{A1} is CH₂, CHMe, or CMe₂; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.
65. The compound of Embodiment 64, wherein each L^{A4} is independently a monocyclic 4-6 membered nitrogen-containing heterocyclylene optionally substituted with 1-3 R^{a},
wherein each L^{A4} contains 1-2 ring nitrogen atoms and no additional ring heteroatoms.
66. The compound of Embodiment 64, wherein one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.
67. The compound of Embodiment 64, wherein one L^{A4} is a monocyclic 4-6 membered nitrogen-containing heterocyclylene optionally substituted with 1-3 R^{a}; and
the other L^{A4} is a bicyclic bridged 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.
68. The compound of any one of Embodiments 64-67, wherein L is -L^{A4}-L^{A1}-L^{A4}-_{bb}; and L^{A1} is CH₂ or CHMe.
69. The compound of Embodiment 64, wherein L is selected from the group consisting of the moieties delineated in Table L1-a:

TABLE L1-a

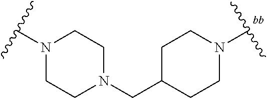

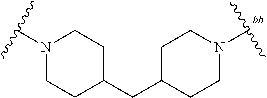

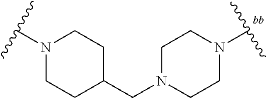

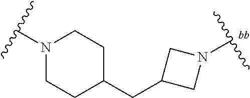

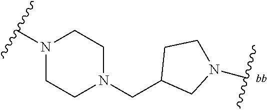

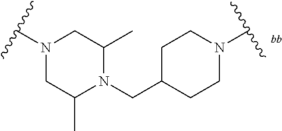

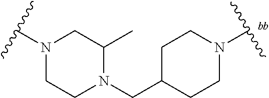

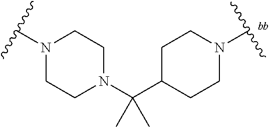

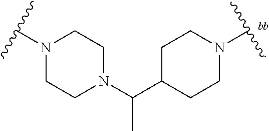

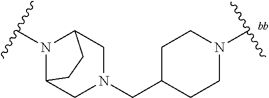

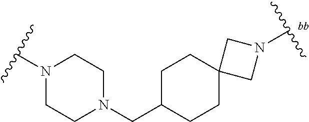

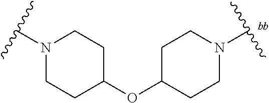

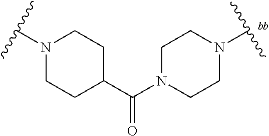

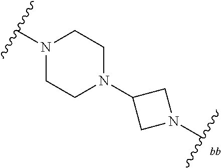

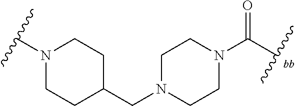

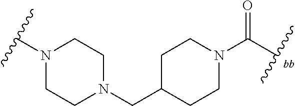

wherein ^{bb} represents the point of attachment to Ring C.

70. The compound of Embodiment 1, wherein the compound is a compound of Formula (I-b):

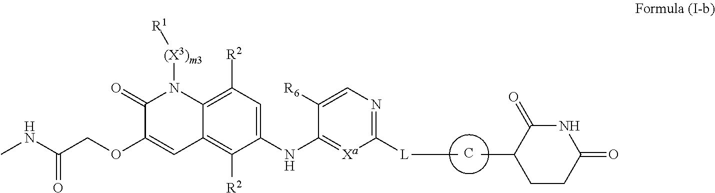

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

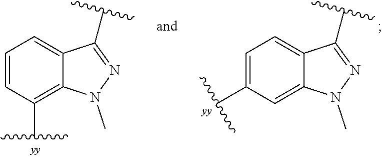 [SMILES: *c1nn(C)c2c(*)cccc12 ; *c1ccc2c(*)nn(C)c2c1]

L is -L^{A4}-L^{A3}-_{bb} or -L^{A4}-L^{A1}-L^{A3}-_{bb}, wherein bb represents the point of attachment to Ring C; and
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.
71. The compound of Embodiment 70, wherein L is -L^{A4}-L^{A3}-_{bb}, and L^{A3} is —NH—.
72. The compound of Embodiments 70 or 71, wherein L^{A4} is a monocyclic 4-6 membered nitrogen-containing heterocyclylene optionally substituted with 1-3 R^{a},
wherein L^{A4} contains 1-2 ring nitrogen atoms and no additional ring heteroatoms.
73. The compound of Embodiments 70 or 71, wherein L^{A4} is a bicyclic spirocyclic 6-12 (e.g., 6-10) membered nitrogen-containing heterocyclylene optionally substituted with 1-3 R^{a},
wherein L^{A4} contains 1-2 ring nitrogen atoms and no additional ring heteroatoms.
74. The compound of Embodiment 70, wherein L is selected from the group consisting of the moieties delineated in Table L2-a:

TABLE L2-a

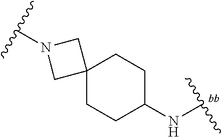

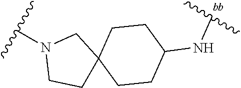

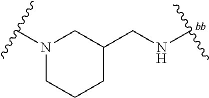

wherein ^{bb} represents the point of attachment to Ring C.

75. The compound of any one of Embodiments 64-74, wherein X^{a} is N.
76. The compound of any one of Embodiments 64-74, wherein X^{a} is CH.
77. The compound of Embodiment 1, wherein the compound is selected from the group consisting of Compound No. 101, 102, 103, 104, 105, 106, 106a, 106b, 107, 108, 260, 260a, 261, 261a, 261b, 262, 262a, 262b, 263, 265, 266, 266a, 266b, 275, 275b, 277, 277b, 279, 279a, 279b, 280, 280a, 281, 282, 283, 284, 286, 287, 288, 289, 289b, 290, 290a, 290b, 292, 293, 293a, 293b, 294, 294a, 294b, 295, 295a, 295b, 296, 297, 297a, 297b, 298, 299, 299a, 300, 300a, 301, 301a, 301b, 302, 302a, 302b, 303, 303a, 303b, 304, 304a, 304b, 305, 305a, 305b, 306, 306a, 306b, 307, 307a, 307b, 308, 308a, 308b, 309, 309a, 309b, 310, 311, 312, 312a, 313, 313a, 314, 315, 316, 317, 317a, 318, 319, 320, 321, 322, 323, 324, 325, 326, 327, 328, 329, 330, 331, 332, 333, 334, 335, 336, 336a, 337, 338, 339, 340, 341, 342, 343, and 344 as depicted in Table C1, or a pharmaceutically acceptable salt thereof.
78. A pharmaceutical composition comprising a compound of any one of Embodiments 1-77, or a pharmaceutically acceptable salt thereof, and a pharmaceutically acceptable excipient.
79. A BCL6 protein non-covalently bound with a compound of any one of Embodiments 1-77, or a pharmaceutically acceptable salt thereof.
80. A ternary complex comprising a BCL6 protein, a compound of any one of Embodiments 1-77, or a pharmaceutically acceptable salt thereof, and a CRBN protein.
81. A method for treating a cancer in a subject in need thereof, the method comprising administering to the subject a therapeutically effective amount of a compound of any one of Embodiments 1-77, or a pharmaceutically acceptable salt thereof, or a pharmaceutical composition according to Embodiment 78.
82. The method of Embodiment 81, wherein the cancer is a hematological cancer, breast cancer, gastrointestinal cancer, brain cancer, lung cancer, or a combination thereof.
83. The method of Embodiment 82, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), chronic lymphocytic leukemia (CLL), acute lymphocytic leukemia (ALL), chronic myeloid leukemia (CML), or a combination thereof.
84 The method of Embodiment 83, wherein the hematological cancer is DLBCL.
85. The method of any one of Embodiments 81-84, further comprising administering an additional therapy or therapeutic agent to the subject.
86. The method of Embodiment 85, wherein the additional therapy or therapeutic agent is a PI3K inhibitor, a BTK inhibitor, a JAK inhibitor, a BRaf inhibitor, a MEK inhibitor, a Bcl-2 inhibitor, a Bcl-xL inhibitor, an XPO1 inhibitor, an inhibitor of the polycomb repressive complex 2, an immunomodulatory imide drug, anti-CD19 therapy, anti-CD20 therapy, anti-CD3 therapy, or a combination thereof.
87. A method for treating an autoimmune condition in a subject in need thereof, the method comprising administering to the subject a therapeutically effective amount of a compound of any one of Embodiments 1-77, or a pharmaceutically acceptable salt thereof, or a pharmaceutical composition according to Embodiment 78.
88. A method for treating a lymphoproliferative disorder in a subject in need thereof, the method comprising administering to the subject a therapeutically effective amount of a compound of any one of Embodiments 1-77, or a pharmaceutically acceptable salt thereof, or a pharmaceutical composition according to Embodiment 78.
89. A method for inducing degradation of a BCL6 protein in a mammalian cell, the method comprising contacting the mammalian cell with an effective amount of a compound of any one of Embodiments 1-77, or a pharmaceutically acceptable salt thereof.
90. The method of Embodiment 89, wherein the contacting occurs in vivo.
91. The method of Embodiment 89, wherein the contacting occurs in vitro.
92. The method of any one of Embodiments 89-91, wherein the mammalian cell is a mammalian cancer cell.

### P04 Embodiments

Embodiment 1. A compound of Formula (I):

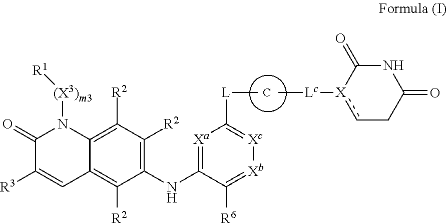

or a pharmaceutically acceptable salt thereof, wherein:
R¹ is selected from the group consisting of: H, halo, cyano, and R^{b1};
m3 is 0, 1, 2, or 3;
each X³ is independently selected from the group consisting of: —O—, —NR^{f}—, —C(═O)—, and C₁₋₃ alkylene optionally substituted with 1-3 R^{c};
provided that the N—(X³)ₘ₃—R¹ moiety does not contain any O—O, N—O, N—N, O-halo, or N-halo bonds;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e},
R³ is selected from the group consisting of:
-A¹-C(R⁴R⁴)-A² and —CH═CH-A², wherein:
A¹ is —O— or —S—;
each R⁴ is independently selected from the group consisting of: H, C₁₋₆ alkyl, and C₁₋₆ haloalkyl; or
the pair of R⁴ taken together with the carbon atom to which each is attached form a C₃₋₆ cycloalkyl ring or a 4-8 membered heterocyclyl ring, wherein the C₃₋₆ cycloalkyl ring or 4-8 membered heterocyclyl ring is optionally substituted with 1-3 R^{g};
A² is selected from the group consisting of: —C(O)OH, —C(O)NH₂, —C(O)R^{3A}, —C(O)OR^{3A}, —C(O)NR^{3A}R^{f}, —S(O)₁₋₂(C₁₋₆ alkyl), —P(O)—(C₁₋₆ alkyl)₂, and —C(═NH)NH₂, wherein:
R^{3A} is selected from the group consisting of: C₁₋₆ alkyl, C₃₋₆ alkenyl, C₃₋₆ alkynyl, C₃₋₆ cycloalkyl, and 3-8 membered heterocyclyl, each of which is optionally substituted with 1-6 substituents independently selected from the group consisting of R^{a} and —(C₀₋₃ alkylene)-R^{b1},
X^{a} and X^{c} are independently selected from the group consisting of: N, CH, and CF, provided that one or both of X^{a} and X^{c} is N;
X^{b} is selected from the group consisting of N and CR^{x1};
R⁶ and R^{x1} are each independently selected from the group consisting of: H, halo, C₁₋₂ alkyl, C₁₋₂ haloalkyl, C₁₋₂ alkoxy, CN, and —C≡CH;
L is -(L^{A})ₙ₁-, wherein L^{A} and n1 are defined according to (AA) or (BB):

### (AA)

n1 is an integer from 1 to 15; and
each L^{A} is independently selected from the group consisting of: L^{A1}, L^{A3}, and L^{A4} provided that 1-3 occurrences of L^{A} is L^{A4}.

### (BB)

n1 is an integer from 0 to 20; and
each L^{A} is independently selected from the group consisting of: L^{A1} and L^{A3},
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O), or O—S(O)₀₋₂ bonds;
wherein each R^{L} is independently selected from the group consisting of: halo, cyano, —OH, —C₁₋₆ alkoxy, —C₁₋₆ haloalkoxy, —NR^{d}R^{e}, C(═O)N(R^{f})₂, S(O)₀₋₂(C₁₋₆ alkyl), S(O)₀₋₂(C₁₋₆ haloalkyl), S(O)₁₋₂N(R^{f})₂, —R^{b}, and C₁₋₆ alkyl optionally substituted with 1-6 R^{c};
Ring C is selected from the group consisting of:

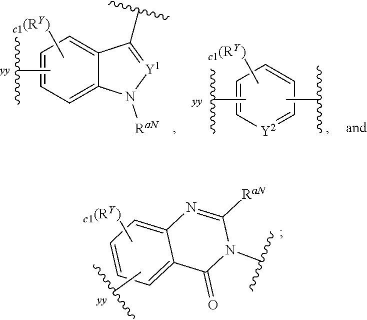

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CRY;
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —C(═O)—, —N(R^{d})—, and O, provided that when X is N, then L^{C} is other than O; and
further provided that when Ring C is attached to -L^{C}- via a ring nitrogen, then X is CH, and L^{C} is a bond;
each R^{a} is independently selected from the group consisting of:
(a) halo;
(b) cyano;
(c) —OH;
(d) oxo;
(e) C₁₋₆ alkoxy optionally substituted with 1-6 R^{c};
(f) —NR^{d}R^{e};
(g) C(═O)C₁₋₆ alkyl optionally substituted with 1-6 R^{c};
(h) C(═O)OH;
(i) C(═O)OC₁₋₆ alkyl;
(j) C(═O)OC₁₋₆ haloalkyl;
(k) C(═O)N(R^{f})₂;
(l) S(O)₀₋₂(C₁₋₆ alkyl);
(m) S(O)₀₋₂(C₁₋₆ haloalkyl);
(n) S(O)₁₋₂N(R^{f})₂; and
(o) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—, —N(H)—, —N(C₁₋₃ alkyl)-, —S(O)₀₋₂—, C(═O), and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo, cyano, —OH, —C₁₋₆ alkoxy, —C₁₋₆ haloalkoxy, —NR^{d}R^{e}, C(═O)C₁₋₆ alkyl, C(═O)C₁₋₆ haloalkyl, C(═O)OC₁₋₆ alkyl, C(═O)OC₁₋₆ haloalkyl, C(═O)OH, C(═O)N(R^{f})₂, S(O)₀₋₂(C₁₋₆ alkyl), S(O)₀₋₂(C₁₋₆ haloalkyl), and S(O)₁₋₂N(R^{f})₂;
each R^{d} and R^{e} is independently selected from the group consisting of: H, C(═O)C₁₋₆ alkyl, C(—O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}, oxo, C₁₋₃ alkyl, and C₁₋₃ haloalkyl; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂,
provided that when X is N, then Ring C is

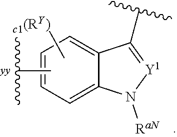

Embodiment 2. The compound of Embodiment 1, wherein R³ is -A¹-C(R⁴R⁴)-A².
Embodiment 3. The compound of Embodiments 1 or 2, wherein A¹ is —O—.
Embodiment 4. The compound of any one of Embodiments 1-3, wherein each R⁴ is H.
Embodiment 5. The compound of any one of Embodiments 1-4, wherein A² is —C(O)NH₂ or —C(O)NR^{3A}R^{f}.
Embodiment 6. The compound of Embodiment 5, wherein R^{3A} is C₁₋₃ alkyl optionally substituted with 1-6 R^{c}
Embodiment 7. The compound of Embodiments 1-6, wherein A² is —C(O)NH₂, —C(O)NHMe, or —C(O)NMe₂.
Embodiment 8. The compound of Embodiment 1, wherein R³ is

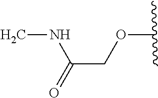 [SMILES: *OCC(=O)NC]

Embodiment 9. The compound of any one of Embodiments 1-8, wherein X^{a} is N; X^{c} is N; and X^{b} is CR^{x1} (e.g., CH).
Embodiment 10. The compound of any one of Embodiments 1-8, wherein X^{a} is CH; X^{c} is N; and X^{b} is CR^{x1} (e.g., CH).
Embodiment 11. The compound of any one of Embodiments 1-10, wherein R⁶ is —Cl or —F.
Embodiment 12. The compound of any one of Embodiments 1-11, wherein each R² is H.
Embodiment 13. The compound of any one of Embodiments 1-11, wherein one R² is selected from the group consisting of: halo, cyano, C₁₋₃ alkyl, C₁₋₃ haloalkyl, C₁₋₃ alkoxy, and C₁₋₃ haloalkoxy; and each remaining R² is H.
Embodiment 14. The compound of any one of Embodiments 1-13, wherein m3 is 0.
Embodiment 15. The compound of any one of Embodiments 1-13, wherein m3 is 1; and X³ is C₁₋₃ alkylene.
Embodiment 16. The compound of any one of Embodiments 1-15, wherein R¹ is H.
Embodiment 17. The compound of any one of Embodiments 1-13, wherein m3 is 1; X³ is methylene, ethylene, or isopropylene; and R¹ is H.
Embodiment 18. The compound of Embodiment 1, wherein:
R³ is -A¹-C(R⁴R⁴)-A², wherein A¹ is O; each R⁴ is H; and A² is —C(O)NH₂ or —C(O)NR^{3A}R^{f}, wherein R^{3A} is C₁₋₃ alkyl optionally substituted with 1-6 R^{c};
each R² is H;
X^{a} is N or CH;
X^{c} is N;
X^{b} is CH; and
R⁶ is —F or —Cl.
Embodiment 19. The compound of Embodiment 18, wherein m3 is 1; X³ is methylene, ethylene, or isopropylene; and R¹ is H.
Embodiment 20. The compound of Embodiments 18 or 19, wherein X^{a} is N.
Embodiment 21. The compound of any one of Embodiments 18-20, wherein A² is C(O)NHMe.
Embodiment 22. The compound of any one of Embodiments 1-21, wherein Ring C is

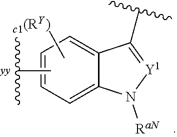

Embodiment 23. The compound of Embodiments 1-22, wherein Ring C is

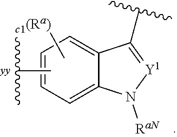

Embodiment 24. The compound of Embodiments 1-22, wherein Ring C is

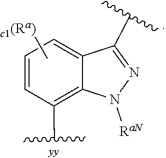

Embodiment 25. The compound of any one of Embodiments 1-21, wherein Ring C is

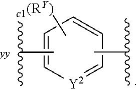

Embodiment 26. The compound of any one of Embodiments 1-25, wherein c1 is 0.
Embodiment 27. The compound of any one of Embodiments 1-25, wherein c1 is 1; and R^{Y} is halo (e.g., —F).
Embodiment 28. The compound of anyone of Embodiments 1-27, wherein X is CH.
Embodiment 29. The compound of any one of Embodiments 1-24 or 27-28, wherein R^{aN} is C₁₋₃ alkyl (e.g., methyl).
Embodiment 30. The compound of any one of Embodiments 1-29, wherein L^{C} is a bond.
Embodiment 31. The compound of any one of Embodiments 1-21, wherein the

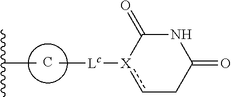

moiety is selected from the group consisting of:

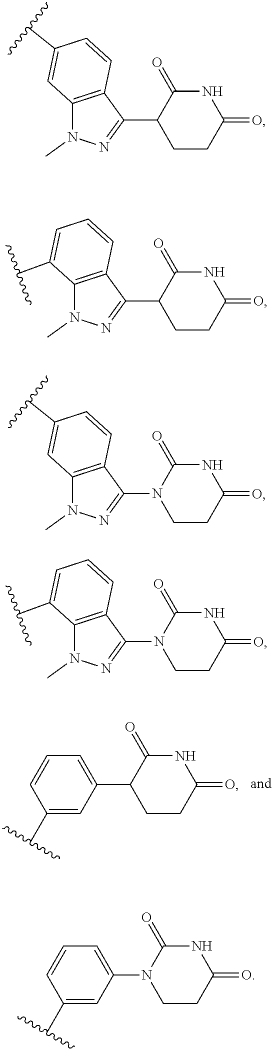

Embodiment 32. The compound of any one of Embodiments 1-21 or 31, wherein the

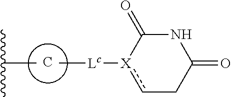

moiety is selected from the group consisting of:

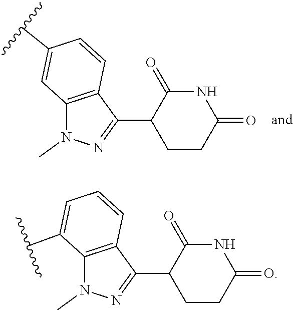

Embodiment 33. The compound of Embodiment 1, wherein the

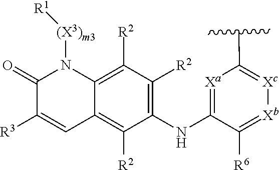

moiety is

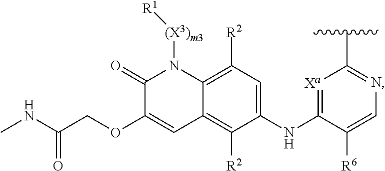

wherein:
m3 is 1;
X³ is C₁₋₃ alkylene;
R¹ is H;
X^{a} is CH or N;
X^{c} is N;
R⁶ is —F or —Cl; and
the

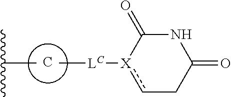

moiety is selected from the group consisting of:

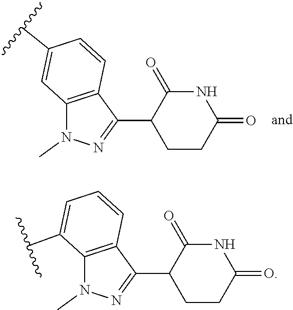

Embodiment 34. The compound of Embodiment 33, wherein —(X³)ₘ₃—R¹ is methyl, ethyl, or isopropyl.
Embodiment 35. The compound of Embodiments 33 or 34, wherein X^{a} is CH.
Embodiment 36. The compound of Embodiments 33 or 34, wherein X^{a} is N.
Embodiment 37. The compound of any one of Embodiments 1-36, wherein L is -(L^{A})ₙ₁-, wherein L^{A} and n1 are defined according to (AA).
Embodiment 38. The compound of any one of Embodiments 1-37, wherein n1 is an integer from 1 to 5.
Embodiment 39. The compound of any one of Embodiments 1-38, wherein n1 is an integer from 2 to 4 (e.g., 2 or 3).
Embodiment 40. The compound of any one of Embodiments 1-39, wherein L is selected from the group consisting of:
-L^{A4}-L^{A1}-L^{A4}-_{bb};
-L^{A4}-L^{A4}-_{bb};
-L^{A4}-L^{A3}-L^{A4}-_{bb}; and
-L^{A4}-L^{A1}-L^{A4}-L^{A3}-_{bb},
wherein bb represents the point of attachment to Ring C.
Embodiment 41. The compound of Embodiment 40, wherein each L^{A4} is independently a C₃₋₁₀ cycloalkylene or 4-12 membered heterocyclylene, each of which is optionally substituted with 1-6 R^{a}
Embodiment 42. The compound of Embodiments 40 or 41, wherein each L^{A4} is independently a 4-12 (e.g., 4-10) membered nitrogen-containing heterocyclylene optionally substituted with 1-3 R^{a}.
Embodiment 43. The compound of any one of Embodiments 40-42, wherein each L^{A4} is independently a monocyclic 4-6 membered nitrogen-containing heterocyclylene optionally substituted with 1-3 R^{a}.
Embodiment 44. The compound of any one of Embodiments 40-42, wherein one L^{A4} is a monocyclic 4-6 membered nitrogen-containing heterocyclylene optionally substituted with 1-3 R^{a}; and the other L^{A4} is a bicyclic 6-12 (e.g., 6-10) membered nitrogen-containing heterocyclylene optionally substituted with 1-3 R^{a}.
Embodiment 45. The compound of any one of Embodiments 40-42 or 44, wherein one L^{A4} is a monocyclic 4-6 membered nitrogen-containing heterocyclylene optionally substituted with 1-3 R^{a}; and the other L^{A4} is a bicyclic spirocyclic 6-12 (e.g., 6-10) membered nitrogen-containing heterocyclylene optionally substituted with 1-3 R^{a}.
Embodiment 46. The compound of any one of Embodiments 40-42 or 44, wherein one L^{A4} is a monocyclic 4-6 membered nitrogen-containing heterocyclylene optionally substituted with 1-3 R^{a}; and the other L^{A4} is a bicyclic bridged 6-12 (e.g., 6-10) membered nitrogen-containing heterocyclylene optionally substituted with 1-3 R^{a}.
Embodiment 47. The compound of any one of Embodiments 42-46, wherein each L^{A4} contains 1-2 ring nitrogen atoms and no additional ring heteroatoms.
Embodiment 48. The compound of any one of Embodiments 41-47, wherein each R^{a} present on L^{A4} is independently selected from the group consisting of: —F, CN, C₁₋₃ alkoxy, OH, and C₁₋₃ alkyl optionally substituted with 1-3 F.
Embodiment 49. The compound of any one of Embodiments 40-48, wherein L is -L^{A4}-L^{A1}-L^{A4}-_{bb}.
Embodiment 50. The compound of Embodiment 49, wherein L^{A1} is —CH₂—, —CHMe-, or —CMe₂-.
Embodiment 51. The compound of any one of Embodiments 40-48, wherein L is -L^{A4}-L^{A3}-L^{A4}-_{bb}.
Embodiment 52. The compound of Embodiment 51, wherein L^{A3} is —C(═O) or —O—.
Embodiment 53. The compound of any one of Embodiments 40-48, wherein L is -L^{A4}-L^{A1}-L^{A4}-L^{A3}-_{bb}, and L^{A3} is C(═O).
Embodiment 54. The compound of any one of Embodiments 1-39, wherein L is selected from the group consisting of:
-L^{A4}-L^{A3}-_{bb};
-L^{A4}-L^{A1}-_{bb}; and
-L^{A4}-L^{A1}-L^{A3}-_{bb},
wherein bb represents the point of attachment to Ring C.
Embodiment 55. The compound of Embodiment 54, wherein L is -L^{A4}-L^{A3}-_{bb}.
Embodiment 56. The compound of Embodiment 55, wherein L^{A3} is —NH— or —N(C₁₋₃ alkyl)- (e.g., —NH—).
Embodiment 57. The compound of any one of Embodiments 54-56, wherein L^{A4} is a 4-12 membered heterocyclylene optionally substituted with 1-6 R^{a}.
Embodiment 58. The compound of any one of Embodiments 54-57, wherein L^{A4} is a 4-12 (e.g., 6-10) membered nitrogen-containing heterocyclylene optionally substituted with 1-3 R^{a}
Embodiment 59. The compound of any one of Embodiments 54-58, wherein L^{A4} is a bicyclic spirocyclic 6-12 (e.g., 6-10) membered nitrogen-containing heterocyclylene optionally substituted with 1-3 R^{a}.
Embodiment 60. The compound of any one of Embodiments 57-59, wherein L^{A4} contains 1-2 ring nitrogen atoms and no additional ring heteroatoms.
Embodiment 61. The compound of any one of Embodiments 57-60, wherein each R^{a} present on L^{A4} is independently selected from the group consisting of: —F, CN, C₁₋₃ alkoxy, OH, and C₁₋₃ alkyl optionally substituted with 1-3 F.
Embodiment 62. The compound of any one of Embodiments 1-40, wherein L is selected from the group consisting of the moieties delineated in Table L1-a:

TABLE L1-a

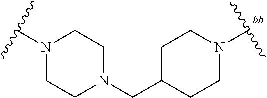

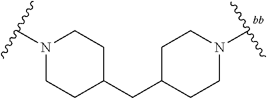

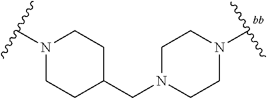

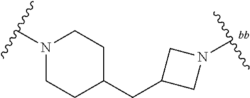

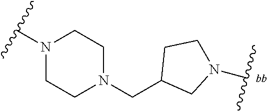

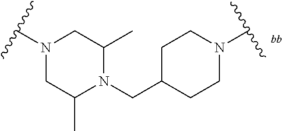

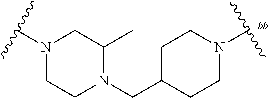

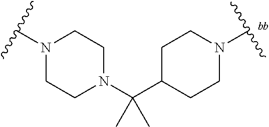

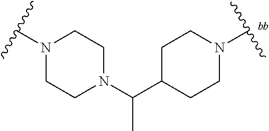

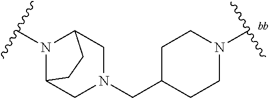

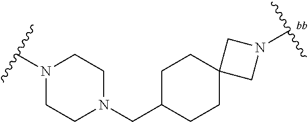 [SMILES: *N1CCN(CC2CCC3(CC2)CN(*)C3)CC1]

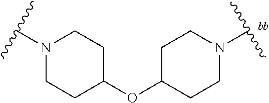

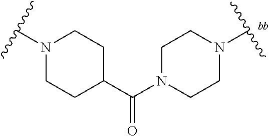

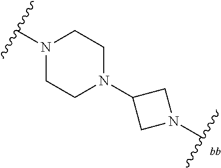

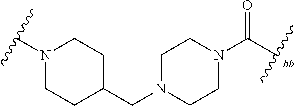

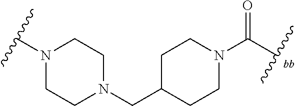

wherein ^{bb} represents the point of attachment to Ring C.

Embodiment 63. The compound of any one of Embodiments 1-39 or 54, wherein L is selected from the group consisting of the moieties delineated in Table L2-a:

TABLE L2-a

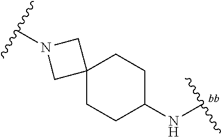

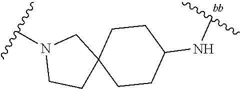

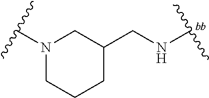

wherein ^{bb} represents the point of attachment to Ring C.

Embodiment 64. The compound of Embodiment 1, wherein the compound is a compound of Formula (I-a):

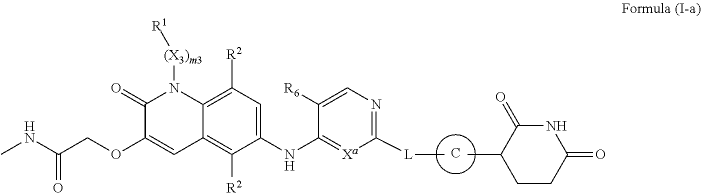

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

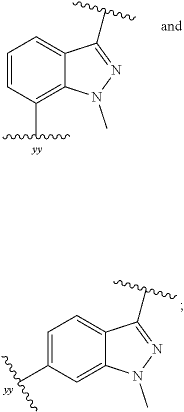 [SMILES: *c1nn(C)c2c(*)cccc12 ; *c1ccc2c(*)nn(C)c2c1]

L is selected from the group consisting of:
-L^{A4}-L^{A1}-L^{A4}-_{bb};
-L^{A4}-O-L^{A4}-_{bb};
-L^{A4}-C(═O)-L^{A4}-_{bb}; and
-L^{A4}-L^{A1}-L^{A4}-C(═O)-_{bb},
wherein bb represents the point of attachment to Ring C;
L^{A1} is CH₂, CHMe, or CMe₂; and
each L^{A4} is independently a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.
Embodiment 65. The compound of Embodiment 64, wherein each L^{A4} is independently a monocyclic 4-6 membered nitrogen-containing heterocyclylene optionally substituted with 1-3 R^{a},
wherein each L^{A4} contains 1-2 ring nitrogen atoms and no additional ring heteroatoms.
Embodiment 66. The compound of Embodiment 64, wherein one L^{A4} is a monocyclic 4-6 membered nitrogen-containing heterocyclylene optionally substituted with 1-3 R^{a}; and
the other L^{A4} is a bicyclic spirocyclic 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.
Embodiment 67. The compound of Embodiment 64, wherein one L^{A4} is a monocyclic 4-6 membered nitrogen-containing heterocyclylene optionally substituted with 1-3 R^{a}; and
the other L^{A4} is a bicyclic bridged 6-12 (e.g., 6-10) membered nitrogen-containing heterocyclylene optionally substituted with 1-3 R^{a},
wherein each L^{A4} contains 1-2 ring nitrogen atoms and no additional ring heteroatoms.
Embodiment 68. The compound of any one of Embodiments 64-67, wherein L is -L^{A4}-L^{A1}-L^{A4}-_{bb}; and L^{A1} is CH₂ or CHMe.
Embodiment 69. The compound of Embodiment 64, wherein L is selected from the group consisting of the moieties delineated in Table L1-a:

TABLE L1-a

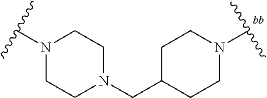

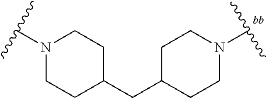

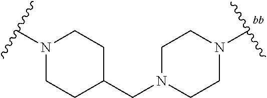

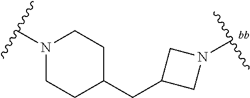

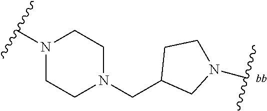

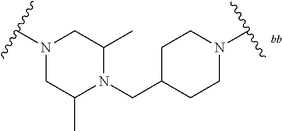

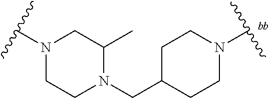

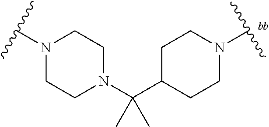

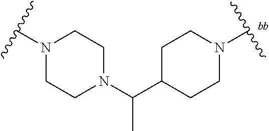

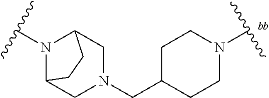

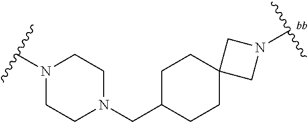 [SMILES: *N1CCN(CC2CCC3(CC2)CN(*)C3)CC1]

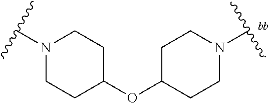

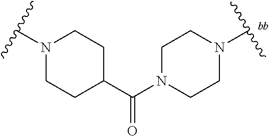

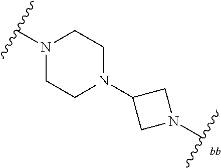

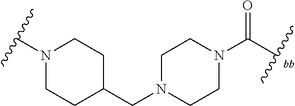

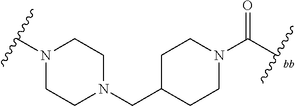

wherein ^{bb} represents the point of attachment to Ring C.

Embodiment 70. The compound of Embodiment 1, wherein the compound is a compound of Formula (I-b):

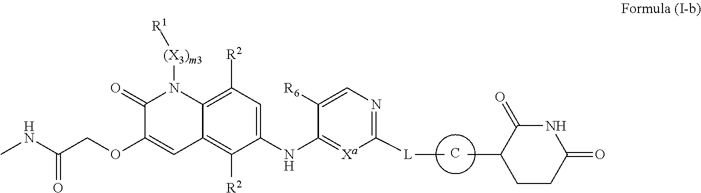

or a pharmaceutically acceptable salt thereof, wherein:
X^{a} is N or CH;
R⁶ is —F or —Cl;
m3 is 1, X³ is C₁₋₃ alkylene, and R¹ is H;
Ring C is selected from the group consisting of:

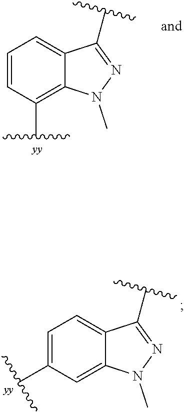 [SMILES: *c1nn(C)c2c(*)cccc12 ; *c1ccc2c(*)nn(C)c2c1]

L is -L^{A4}-L^{A3}-_{bb} or -L^{A4}-L^{A1}-L^{A3}-_{bb}, wherein bb represents the point of attachment to Ring C; and
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F.
Embodiment 71. The compound of Embodiment 70, wherein L is -L^{A4}-L^{A3}-_{bb}, and L^{A3} is —NH—.
Embodiment 72. The compound of Embodiments 70 or 71, wherein L^{A4} is a monocyclic 4-6 membered nitrogen-containing heterocyclylene optionally substituted with 1-3 R^{a},
wherein L^{A4} contains 1-2 ring nitrogen atoms and no additional ring heteroatoms.
Embodiment 73. The compound of Embodiments 70 or 71, wherein L^{A4} is a bicyclic spirocyclic 6-12 (e.g., 6-10) membered nitrogen-containing heterocyclylene optionally substituted with 1-3 R^{a},
wherein L^{A4} contains 1-2 ring nitrogen atoms and no additional ring heteroatoms.
Embodiment 74. The compound of Embodiment 70, wherein L is selected from the group consisting of the moieties delineated in Table L2-a:

TABLE L2-a

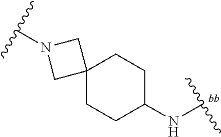

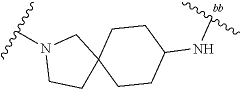

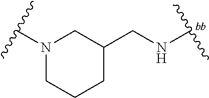

wherein ^{bb} represents the point of attachment to Ring C.

Embodiment 75. The compound of any one of Embodiments 64-74, wherein X^{a} is N.
Embodiment 76. The compound of any one of Embodiments 64-74, wherein X^{a} is CH.
Embodiment 77. The compound of Embodiment 1, wherein the compound is selected from the group consisting of Compound No. 101, 102, 103, 104, 105, 106, 106a, 106b, 107, 108, 260, 260a, 260b, 261, 261a, 261b, 262, 262a, 262b, 263, 265, 266, 266a, 266b, 275, 275a, 275b, 277, 277a, 277b, 279, 279a, 279b, 280, 280a, 280b, 281, 282, 283, 284, 286, 287, 288, 289, 289a, 289b, 290, 290a, 290b, 292, 293, 293a, 293b, 294, 294a, 294b, 295, 295a, 295b, 296, 297, 297a, 297b, 298, 299, 299a, 300, 300a, 300b, 301, 301a, 301b, 302, 302a, 302b, 303, 303a, 303b, 304, 304a, 304b, 305, 305a, 305b, 306, 306a, 306b, 307, 307a, 307b, 308, 308a, 308b, 309, 309a, 309b, 310, 311, 312, 312a, 313, 313a, 314, 315, 316, 317, 317a, 318, 319, 320, 321, 322, 323, 324, 325, 326, 327, 328, 329, 330, 331, 332, 333, 334, 335, 336, 336a, 337, 338, 339, 340, 341, 342, 343, 344, 345, 346, 347, 347a, and 348 as depicted in Table C1, or a pharmaceutically acceptable salt thereof.
Embodiment 78. A pharmaceutical composition comprising a compound of any one of Embodiments 1-77, or a pharmaceutically acceptable salt thereof, and a pharmaceutically acceptable excipient.
Embodiment 79. A BCL6 protein non-covalently bound with a compound of any one of Embodiments 1-77, or a pharmaceutically acceptable salt thereof.
Embodiment 80. A ternary complex comprising a BCL6 protein, a compound of any one of Embodiments 1-77, or a pharmaceutically acceptable salt thereof, and a CRBN protein.
Embodiment 81. A method for treating a cancer in a subject in need thereof, the method comprising administering to the subject a therapeutically effective amount of a compound of any one of Embodiments 1-77, or a pharmaceutically acceptable salt thereof, or a pharmaceutical composition according to Embodiment 78.
Embodiment 82. The method of Embodiment 81, wherein the cancer is a hematological cancer, breast cancer, gastrointestinal cancer, brain cancer, lung cancer, or a combination thereof.
Embodiment 83. The method of Embodiment 82, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML).
Embodiment 84. The method of Embodiment 83, wherein the hematological cancer is selected from the group consisting of DLBCL, FL, MCL, BL, PTCL, and ALL (e.g., B-ALL).
Embodiment 85. The method of Embodiment 83, wherein the hematological cancer is FL or DLBCL.
Embodiment 86. The method of Embodiment 83, wherein the hematological cancer is DLBCL.
Embodiment 87. The method of Embodiment 83, wherein the hematological cancer is FL.
Embodiment 88. The method of Embodiment 83, wherein the hematological cancer is BL.
Embodiment 89. The method of Embodiment 83, wherein the hematological cancer is a PTCL.
Embodiment 90. The method of Embodiment 83, wherein, the hematological cancer is ALL (e.g., B-ALL).
Embodiment 91. The method of any one of Embodiments 81-90, wherein the therapeutically effective amount of a compound of any one of Embodiments 1-77, or a pharmaceutically acceptable salt thereof, or a pharmaceutical composition according to Embodiment 78, is administered to the subject as a monotherapy.
Embodiment 92. The method of any one of Embodiments 81-90, comprising administering an additional therapy or therapeutic agent to the subject.
Embodiment 93. The method of Embodiment 92, wherein the additional therapy or therapeutic agent is a PI3K inhibitor, an Abl inhibitor (e.g., a BCR-Abl inhibitor), a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, or a combination thereof.
Embodiment 94. The method of Embodiment 93, wherein the additional therapy or therapeutic agent is an Abl inhibitor.
Embodiment 95. The method of Embodiment 94, wherein the cancer is a B-ALL (e.g., a Philadelphia chromosome positive B-ALL or Philadelphia chromosome-like B-ALL).
Embodiment 96. The method of Embodiment 93, wherein the additional therapy or therapeutic agent is a BTK inhibitor.
Embodiment 97. The method of Embodiment 96, wherein the cancer is a B-ALL (e.g., a pre-BCR+B-ALL or a B-ALL dependent on Ras signaling).
Embodiment 98. The method of Embodiment 93, wherein the additional therapy or therapeutic agent is a JAK inhibitor.
Embodiment 99. The method of Embodiment 98, wherein the cancer is a B-ALL (e.g., a JAK2 (e.g., JAK2^{R683G} or JAK2^{I682F}) mutant B-ALL, with or without high CRLF2 expression).
Embodiment 100. The method of Embodiment 93, wherein the additional therapy or therapeutic agent is a BCL-2 inhibitor.
Embodiment 101. The method of Embodiment 100, wherein the cancer is selected from the group consisting of DLBCL, FL, MCL, and ALL (e.g., B-ALL).
Embodiment 102. The method of Embodiment 100, wherein the cancer is a DLBCL.
Embodiment 103. The method of Embodiment 100, wherein the cancer is a FL.
Embodiment 104. The method of Embodiment 100, wherein the cancer is an MCL.
Embodiment 105. The method of Embodiment 100, wherein the cancer is a B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, or a BCL2 amplified B-ALL).
Embodiment 106. The method of Embodiment 93, wherein the additional therapy or therapeutic agent is an inhibitor of the PRC2.
Embodiment 107. The method of Embodiment 106, wherein the cancer is selected from the group consisting of DLBCL, FL, MCL, and ALL (e.g., B-ALL).
Embodiment 108. The method of Embodiment 106, wherein the cancer is a DLBCL.
Embodiment 109. The method of Embodiment 106, wherein the cancer is a FL.
Embodiment 110. The method of Embodiment 106, wherein the cancer is an MCL.
Embodiment 111. The method of Embodiment 106, wherein the cancer is a B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, or a BCL2 amplified B-ALL).
Embodiment 112. The method of Embodiment 93, wherein the additional therapy or therapeutic agent is anti-CD20 therapy.
Embodiment 113. The method of Embodiment 112, wherein the cancer is a DLBCL or a FL.
Embodiment 114. The method of Embodiment 112, wherein the cancer is a DLBCL.
Embodiment 115. The method of Embodiment 112, wherein the cancer is a FL.
Embodiment 116. The method of Embodiment 93, wherein the additional therapy or therapeutic agent is chemotherapy.
Embodiment 117. The method of Embodiment 116, wherein the cancer is B-ALL (e.g., an MLL-rearranged (e.g., an MLL-Af4 fusion, an MLL-Af6 fusion, an MLL-Af9 fusion, an MLL-ENL fusion, or an MLL-PTD fusion) B-ALL, a Philadelphia chromosome positive B-ALL, a Philadelphia chromosome-like B-ALL, a pre-BCR+B-ALL, a B-ALL dependent on Ras signaling).
Embodiment 118. A method for treating an autoimmune condition in a subject in need thereof, the method comprising administering to the subject a therapeutically effective amount of a compound of any one of Embodiments 1-77, or a pharmaceutically acceptable salt thereof, or a pharmaceutical composition according to Embodiment 78.
Embodiment 119. A method for treating a lymphoproliferative disorder in a subject in need thereof, the method comprising administering to the subject a therapeutically effective amount of a compound of any one of Embodiments 1-77, or a pharmaceutically acceptable salt thereof, or a pharmaceutical composition according to Embodiment 78.
Embodiment 120. A method for inducing degradation of a BCL6 protein in a mammalian cell, the method comprising contacting the mammalian cell with an effective amount of a compound of any one of Embodiments 1-77, or a pharmaceutically acceptable salt thereof.
Embodiment 121. The method of Embodiment 120, wherein the contacting occurs in vivo.
Embodiment 122. The method of Embodiment 120, wherein the contacting occurs in vitro.
Embodiment 123. The method of any one of Embodiments 120-122, wherein the mammalian cell is a mammalian cancer cell.

1.-56. (canceled)

57. A compound of Formula (I-bb): 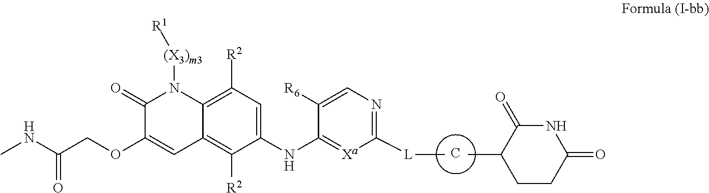
or a pharmaceutically acceptable salt thereof, wherein:
X^{a} is N or CH;
R⁶ is —F or —Cl;
m3 is 1;
X³ is C₁₋₃ alkylene;
R¹ is H;
each R² is independently selected from the group consisting of: H, halo, cyano, C₁₋₃ alkyl, C₁₋₃ haloalkyl, C₁₋₃ alkoxy, C₁₋₃ haloalkoxy, —OH, and —NR^{d}R^{e};
Ring C is selected from the group consisting of: 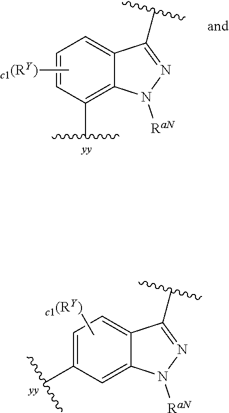
wherein:
c1 is 0 or 1,
R^{Y} is selected from the group consisting of halo and C₁₋₃ alkyl optionally substituted with 1-3 F, and
R^{aN} is C₁₋₃ alkyl;
L is -L^{A4}-L^{A3}-_{bb}, wherein bb represents the point of attachment to Ring C;
L^{A3} is —NH—;
L^{A4} is a 4-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F;
each R^{d} and R^{e} is independently selected from the group consisting of: H, C(═O)C₁₋₆ alkyl, C(═O)C₁₋₆ haloalkyl, C(═O)OC₁₋₆ alkyl, C(═O)OC₁₋₆ haloalkyl, C(═O)N(R^{f})₂, S(O)₁₋₂(C₁₋₆ alkyl), S(O)₁₋₂(C₁₋₆ haloalkyl), S(O)₁₋₂N(R^{f})₂, and C₁₋₆ alkyl optionally substituted with 1-3 R^{h},
each R^{f} is independently selected from the group consisting of: H and C₁₋₆ alkyl optionally substituted with 1-3 R^{h}; and
each R^{h} is independently selected from the group consisting of: halo, cyano, —OH, —(C₀₋₃ alkylene)-C₁₋₆ alkoxy, —(C₀₋₃ alkylene)-C₁₋₆ haloalkoxy, —(C₀₋₃ alkylene)-NH₂, —(C₀₋₃ alkylene)-N(H)(C₁₋₃ alkyl), and —(C₀₋₃ alkylene)-N(C₁₋₃ alkyl)₂.

58. The compound of claim 57, wherein L^{A4} is a monocyclic 4-6 membered nitrogen-containing heterocyclylene optionally substituted with 1-3 R^{a}, wherein L^{A4} contains 1-2 ring nitrogen atoms and no additional ring heteroatoms.

59. The compound of claim 57, wherein L^{A4} is a bicyclic spirocyclic 6-12 membered nitrogen-containing heterocyclylene optionally substituted with 1-3 R^{a}, wherein L^{A4} contains 1-2 ring nitrogen atoms and no additional ring heteroatoms.

60. The compound of claim 57, wherein the compound is a compound of Formula (I-bb-1): 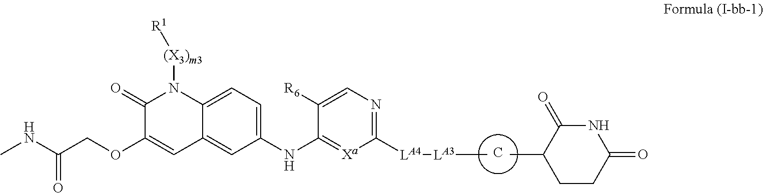
or pharmaceutically acceptable salts thereof, wherein:
X^{a} is N or CH;
R⁶ is —F or —Cl;
m3 is 1;
X³ is C₁₋₃ alkylene;
R¹ is H;
Ring C is selected from the group consisting of: 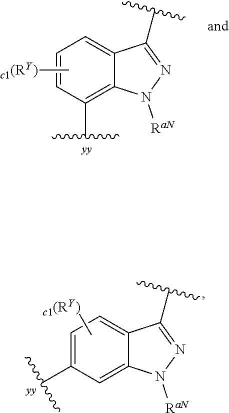
wherein:
c1 is 0 or 1,
R^{Y} is selected from the group consisting of halo and C₁₋₃ alkyl optionally substituted with 1-3 F, and
R^{aN} is C₁₋₃ alkyl;
L^{A4} is a 6-12 membered nitrogen-containing heterocyclylene optionally substituted with 1-3 R^{a}, wherein:
each R^{a} present on L^{A4} is independently selected from the group consisting of: —F, CN, C₁₋₃ alkoxy, OH, and C₁₋₃ alkyl optionally substituted with 1-3 F; and
L^{A3} is —NH—.

61. The compound of claim 60, wherein -L^{A4}-L^{A3}- is selected from the group consisting -L^{A4}-L^{A3}- is selected from the group consisting of: 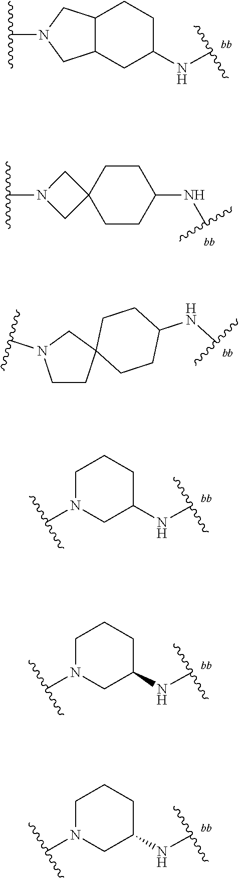
wherein bb represents the point of attachment to Ring C.

62. The compound of claim 57, wherein the compound is a compound of Formula (I-bb-2): 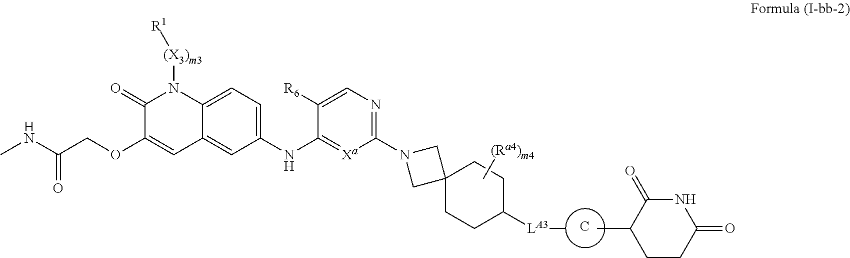
or pharmaceutically acceptable salts thereof, wherein:
X^{a} is N or CH;
R⁶ is —F or —Cl;
m3 is 1;
X³ is C₁₋₃ alkylene;
R¹ is H;
Ring C is selected from the group consisting of: 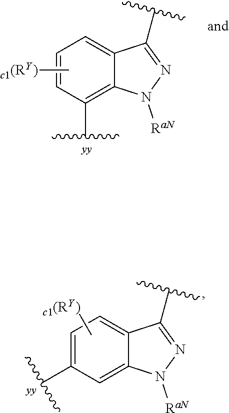
wherein:
c1 is 0 or 1,
R^{Y} is selected from the group consisting of halo and C₁₋₃ alkyl optionally substituted with 1-3 F, and
R^{aN} is C₁₋₃ alkyl;
L^{A3} is —NH—;
m4 is selected from the group consisting of: 0, 1, and 2; and
each R^{a4} is independently selected from the group consisting of: —F, CN, C₁₋₃ alkoxy, OH, and C₁₋₃ alkyl optionally substituted with 1-3 F.

63. The compound of claim 62, wherein m4 is 0 or 1; and R^{a4} when present is methyl.

64. The compound of claim 57, wherein X^{a} is N.

65. The compound of claim 60, wherein X^{a} is N.

66. The compound of claim 62, wherein X^{a} is N.

67. The compound of claim 57, wherein R^{Y} is —F.

68. The compound of claim 57, wherein the compound is selected from the group consisting of: 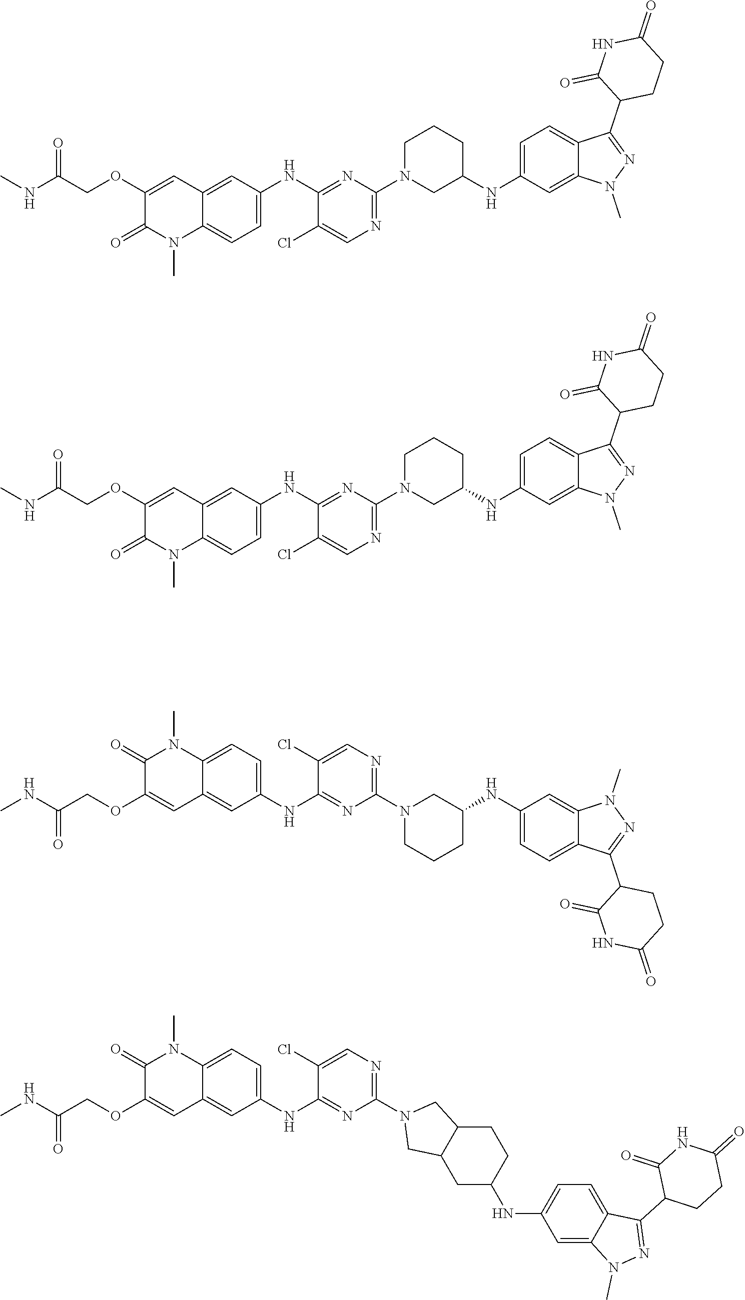 [SMILES: CNC(=O)COc1cc2cc(Nc3nc(N4CCCC(Nc5ccc6c(C7CCC(=O)NC7=O)nn(C)c6c5)C4)ncc3Cl)ccc2n(C)c1=O ; CNC(=O)COc1cc2cc(Nc3nc(N4CCC[C@H](Nc5ccc6c(C7CCC(=O)NC7=O)nn(C)c6c5)C4)ncc3Cl)ccc2n(C)c1=O ; CNC(=O)COc1cc2cc(Nc3nc(N4CCC[C@@H](Nc5ccc6c(C7CCC(=O)NC7=O)nn(C)c6c5)C4)ncc3Cl)ccc2n(C)c1=O ; CNC(=O)COc1cc2cc(Nc3nc(N4CC5CCC(Nc6ccc7c(C8CCC(=O)NC8=O)nn(C)c7c6)CC5C4)ncc3Cl)ccc2n(C)c1=O] 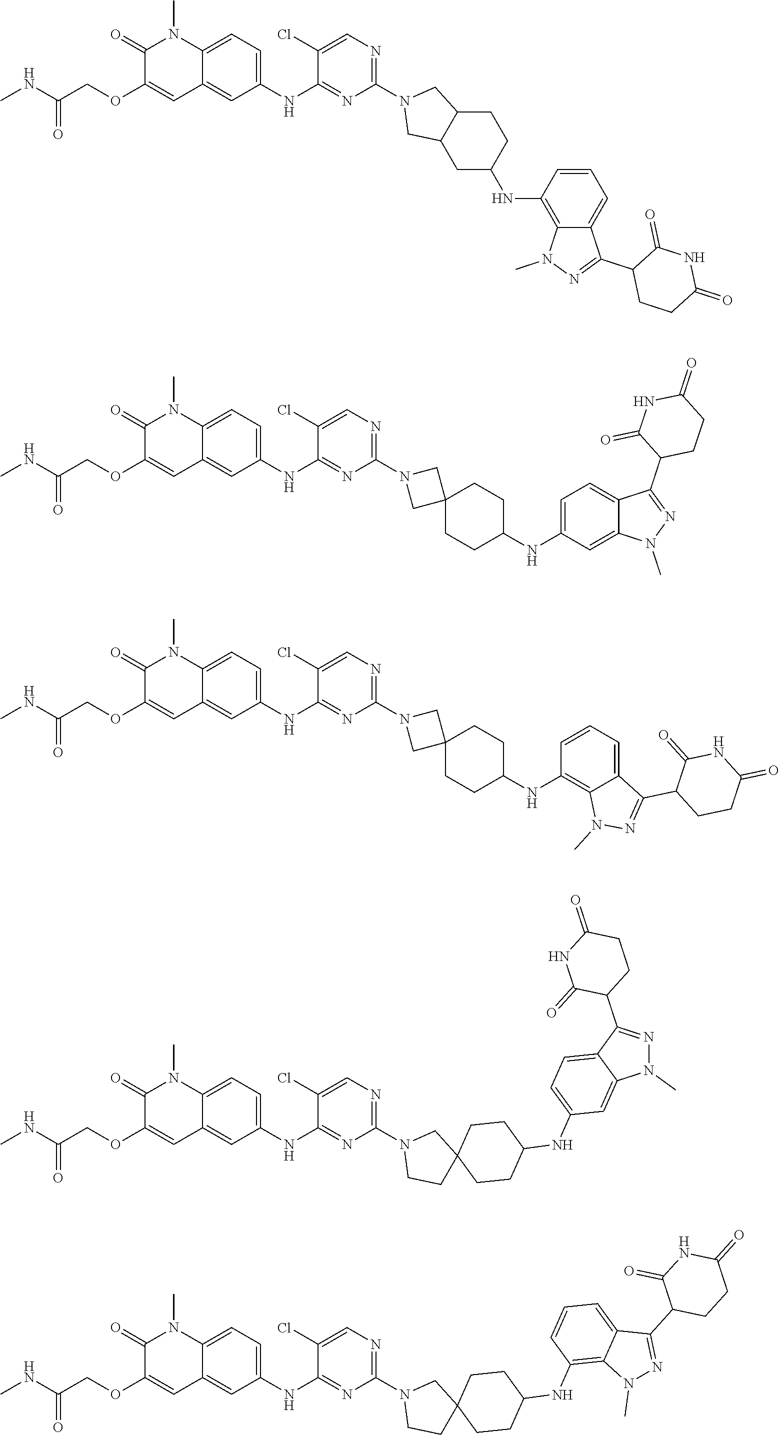
or a pharmaceutically acceptable salt thereof.

69. A pharmaceutical composition comprising a compound of claim 57, or a pharmaceutically acceptable salt thereof, and a pharmaceutically acceptable excipient.

70. A method for treating a cancer in a subject in need thereof, the method comprising administering to the subject a therapeutically effective amount of a compound of claim 57, or a pharmaceutically acceptable salt thereof.

71. The method of claim 70, wherein the cancer is a hematological cancer, breast cancer, gastrointestinal cancer, brain cancer, lung cancer, or a combination thereof.

72. The method of claim 71, wherein the hematological cancer is diffuse large B-cell lymphoma (DLBCL), follicular lymphoma (FL), nodular lymphocyte predominant Hodgkin lymphoma (NLPHL), diffuse histiocytic lymphoma (DHL), intravascular large B-cell lymphoma (IVLBCL), small lymphocytic lymphoma (SLL), Burkitt lymphoma (BL), mantle cell lymphoma (MCL), peripheral T-cell lymphoma (PTCL), chronic lymphocytic leukemia (CLL), acute lymphocytic leukemia (ALL), or chronic myeloid leukemia (CML).

73. The method of claim 70, comprising administering an additional therapy or therapeutic agent to the subject.

74. The method of claim 73, wherein the additional therapy or therapeutic agent is a PI3K inhibitor, an Abl inhibitor, a BTK inhibitor, a JAK inhibitor, a BRaf inhibitor, a MEK inhibitor, a BCL-2 inhibitor, a BCL-XL inhibitor, an XPO1 inhibitor, an inhibitor of the polycomb repressive complex 2 (PRC2), an immunomodulatory imide drug, anti-CD19 therapy, anti-CD20 therapy, anti-CD3 therapy, chemotherapy, or a combination thereof.
